(19) United States
(12) Patent Application Publication
(10) Pub. No.: US 2026/0001978 A1
(43) Pub. Date: Jan. 1, 2026

(54) SULFONIUM SALT MONOMER, POLYMER, CHEMICALLY AMPLIFIED RESIST COMPOSITION, AND PATTERN FORMING PROCESS

(71) Applicant: SHIN-ETSU CHEMICAL CO., LTD., Tokyo (JP)

(72) Inventor: Masahiro Fukushima, Joetsu-shi (JP)

(73) Assignee: SHIN-ETSU CHEMICAL CO., LTD., Tokyo (JP)

(21) Appl. No.: 19/235,811

(22) Filed: Jun. 12, 2025

(30) Foreign Application Priority Data
Jun. 26, 2024 (JP) 2024-103103

(51) Int. Cl. C08F 220/22; C08F 212/14; C08F 220/18; G03F 7/004; G03F 7/039; G03F 7/36

(52) U.S. Cl. CPC C08F 220/22; C08F 212/22; C08F 220/1806; C08F 220/1807; G03F 7/0045; G03F 7/0392; G03F 7/36

(57) ABSTRACT

The sulfonium salt monomer is for a chemically amplified resist composition processed by photolithography using, in particular, high-energy radiation such as KrF excimer laser, ArF excimer laser, an electron beam (EB) or EUV, has excellent solvent solubility and a high sensitivity and contrast, and is excellent in lithographic performance such as exposure tolerance (EL), LWR, CDU and depth of focus (DOF), and excellent in resistance to pattern collapse and etch resistance even in fine pattern formation, a polymer comprising repeat units derived from the sulfonium salt monomer. The sulfonium salt monomer has the formula (a).

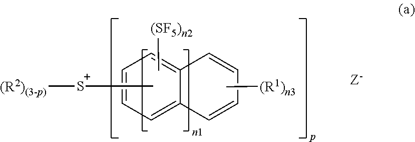

### CROSS-REFERENCE TO RELATED APPLICATION

This non-provisional application claims priority under 35 U.S.C. § 119(a) on Patent Application No. 2024-103103 filed in Japan on Jun. 26, 2024, the entire contents of which are hereby incorporated by reference.

### TECHNICAL FIELD

The present invention relates to a sulfonium salt monomer, a polymer, a chemically amplified resist composition, and a pattern forming process.

### BACKGROUND ART

To meet the demand for higher integration density and operating speed of LSIs, the effort to reduce the pattern rule is in rapid progress. The wide-spreading flash memory market and the demand for increased storage capacities drive forward the miniaturization technology. As the advanced miniaturization technology, manufacturing of microelectronic 65-nm mode devices by the ArF lithography has been implemented in a mass scale.

Manufacturing of 45-nm node devices by the next generation ArF immersion lithography is approaching to the verge of high-volume application. The candidates for the next generation 32-nm node include ultra-high NA lens immersion lithography using a liquid having a higher refractive index than water in combination with a high refractive index lens and a high refractive index resist film, EUV lithography of wavelength 13.5 nm, and double patterning version of the ArF lithography, on which active research efforts have been made.

As the pattern feature size is reduced, approaching to the diffraction limit of light, light contrast lowers. In the case of positive resist film, a lowering of light contrast leads to reductions of resolution and focus margin of hole and trench patterns.

As the pattern feature size is reduced, the line width roughness (LWR) of line patterns and the critical dimension uniformity (CDU) of hole patterns are regarded significant. It is pointed out that these factors are affected by the segregation or agglomeration of a base polymer and acid generator and the diffusion of generated acid. There is a tendency that as the resist film becomes thinner, LWR becomes greater. A film thickness reduction to comply with the progress of size reduction causes a degradation of LWR, which becomes a serious problem.

The EUV lithography resist must meet high sensitivity, high resolution and low LWR at the same time. As the acid diffusion distance is reduced, LWR is reduced, but sensitivity becomes lower. For example, as the PEB temperature is lowered, the outcome is a reduced LWR, but a lower sensitivity. As the amount of quencher added is increased, the outcome is a reduced LWR, but a lower sensitivity. It is necessary to overcome the tradeoff relation between sensitivity and LWR.

Patent Document 1 discloses a resist compound comprising repeat units derived from an onium salt of a polymerizable unsaturated bond-containing sulfonic acid. The so called polymer-bound acid generator is capable of generating a polymer type sulfonic acid upon exposure and characterized by a very short distance of acid diffusion. Sensitivity may be enhanced by increasing a proportion of the acid generator. In the case of addition type acid generators, as the amount of acid generator added is increased, a higher sensitivity is achievable, but the acid diffusion distance is also increased. Since the acid diffusion is non-uniform, increased acid diffusion leads to degraded LWR and CDU. With respect to a balance of sensitivity, LWR and CDU, the polymer-bound acid generator has a high capability.

Since iodine atoms are highly absorptive to EUV of wavelength 13.5 nm, they generate secondary electrons upon light exposure. This effect is noteworthy in the EUV lithography. Patent Document 2 discloses a photoacid generator having iodine atoms in the anion, and Patent Document 3 discloses a polymerizable group-containing photoacid generator having iodine atoms in the anion. Iodine atoms have been confirmed to improve lithographic performance to some extent, but do not have high organic solvent solubility, and may be precipitated in the solvent.

Patent Documents 4 to 7 disclose a resist composition in which a polymer comprising repeat units derived from a weak acid sulfonium salt having a pKa of −0.8 or greater and having a polymerizable group is used as a polymer-bound quencher. Patent Document 4 points to carboxylic acid, sulfonamide, phenol, hexafluoroalcohol or the like as the weak acid.

### CITATION LIST

Patent Document 1: JP 4425776
Patent Document 2: JP 6720926
Patent Document 3: JP 6973274
Patent Document 4: WO 2019/167737
Patent Document 5: WO 2022/264845
Patent Document 6: JP-A 2022-115072
Patent Document 7: JP 7433394

### SUMMARY OF THE INVENTION

In the field of acid-catalyzed chemically amplified resist compositions, it is desired to have a resist composition having a higher sensitivity, improved LWR of line patterns, and improved CDU of hole patterns, and exhibiting excellent etch resistance after formation of patterns.

The present invention has been made in view of the above-described circumstances, and an object of the present invention is to provide a sulfonium salt monomer for a chemically amplified resist composition which is processed by photolithography using, in particular, high-energy radiation such as KrF excimer laser, ArF excimer laser, an electron beam (EB) or EUV, has excellent solvent solubility and a high sensitivity and contrast, and is excellent in lithographic performance such as exposure tolerance (EL), LWR, CDU and depth of focus (DOF), and excellent in resistance to pattern collapse and etch resistance even in fine pattern formation, a polymer comprising repeat units derived from the sulfonium salt monomer, a chemically amplified resist composition containing the polymer, and a pattern forming process using the chemically amplified resist composition.

The present inventors have extensively conducted studies for achieving the above-described object, and resultantly found that by using, as a polymer-bound quencher, a polymer comprising repeat units derived from a sulfonium salt having a styrene or vinyl naphthalene structure as a polymerizable group and containing a carboxylate anion having iodine, and a sulfonium cation having a pentafluorosulfanyl group (—SF₅ group), a chemically amplified resist composition having a high sensitivity, improved lithographic performance such as improved EL, LWR, CDU and DOF, high contrast, high resolution, and excellent etch resistance can be obtained.

The present invention provides the following sulfonium salt monomer, polymer, chemically amplified resist composition, and pattern forming process.
1. A sulfonium salt monomer having the formula (a):

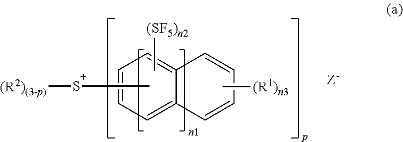

wherein p is 1, 2 or 3, n1 is 0 or 1, n2 is 1 or 2, n3 is 0, 1, 2 or 3, n2+n3 is from 1 to 5 when n1 is 0, n2+n3 is from 1 to 7 when n1 is 1,
   R¹ is halogen, nitro group, cyano group, hydroxy group, a C₁-C₂₀ hydrocarbyl group which may contain a heteroatom, a C₁-C₂₀ hydrocarbyloxy group which may contain a heteroatom, a C₁-C₂₀ hydrocarbyloxy group, or a C₁-C₂₀ hydrocarbylthio group which may contain a heteroatom, a plurality of R¹ may be identical or different and two R¹ may bond together to form a ring with the carbon atoms to which they are attached, when n3 is 2 or 3,
   R² is halogen, or a C₁-C₃₀ hydrocarbyl group which may contain a heteroatom, two R² may be identical or different when p is 1, two of three substituents bonded to S⁺ may bond together to form a ring with a sulfur atom to which they are attached, and Z⁻ is a carboxylate anion having an aromatic vinyl structure and iodine.
2. The sulfonium salt monomer of 1 which has the formula (a1):

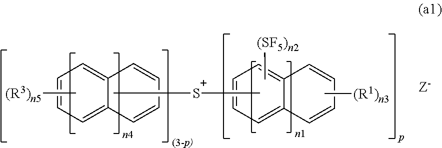

wherein p, n1 to n3, and R¹ and Z⁻ are as defined above,
   n4 is 0 or 1, n5 is 0, 1, 2, 3, 4 or 5,
   R³ is halogen, nitro group, cyano group, hydroxy group, a C₁-C₂₀ hydrocarbyl group which may contain a heteroatom, a C₁-C₂₀ hydrocarbyloxy group which may contain a heteroatom, or a C₁-C₂₀ hydrocarbyloxy group, a C₁-C₂₀ hydrocarbylthio group which may contain a heteroatom, and a plurality of R³ may be identical or different and two R³ may bond together to form a ring with the carbon atoms to which they are attached, when n5 is an integer of 2 to 5.
3. The sulfonium salt monomer of 1 or 2, wherein Z⁻ is an anion having the formula (Z):

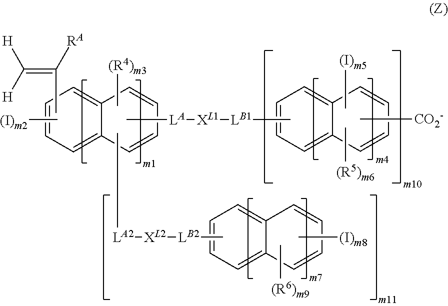

wherein m1 is 0 or 1, m2 is 0, 1, 2, 3 or 4, m3 is 0, 1, 2 or 3, m4 is 0 or 1, m5 is 0, 1, 2, 3 or 4, m6 is 0, 1, 2 or 3, m7 is 0 or 1, m8 is 1, 2, 3 or 4, m9 is 0, 1, 2 or 3, m10 is 0 or 1, m1 is or 1, m2+m3+m1 lis from 0 to 4 when m1 is 0, m2+m3+m1 lis from 0 to 6 when m1 is 1, m5+m6 is from 0 to 4 when m4 is 0, m5+m6 is from 0 to 6 when m4 is 1, m8+m9 is from 0 to 5 when m7 is 0, m8+m9 is from 0 to 7 when m7 is 1, m2+m5+m8 is from 1 to 4,
   R^{A} is hydrogen, fluorine, methyl group or trifluoromethyl group,
   R⁴, R⁵ and R⁶ are each independently halogen exclusive of iodine, nitro group, hydroxy group, a C₁-C₂₀ hydrocarbyl group which may contain a heteroatom, a C₁-C₂₀ hydrocarbyloxy group which may contain a heteroatom, or a C₁-C₂₀ hydrocarbyloxy group, a C₁-C₂₀ hydrocarbylthio group which may contain a heteroatom, a plurality of R⁴ may be identical or different and two R⁴ may bond together to form a ring with the carbon atoms to which they are attached, when m3 is 2 or 3, a plurality of R⁵ may be identical or different and two R⁵ may bond together to form a ring with the carbon atoms to which they are attached, when m6 is 2 or 3, a plurality of R⁶ may be identical or different and two R⁶ may bond together to form a ring with the carbon atoms to which they are attached, when m9 is 2 or 3,
   L^{A1}, L^{A2} and L^{B1} and L^{B2} are each independently a single bond, ether bond, carbonyl group, ester bond, sulfonic ester bond, amide bond, sulfonic amide bond, carbonate bond or carbamate bond,
   X^{L1} and X^{L2} are each independently a single bond, or a C₁-C₄₀ hydrocarbylene group which may contain a heteroatom.
4. A sulfonium salt quencher comprising the sulfonium salt monomer of any one of 1 to 3.
5. A polymer comprising repeat units derived from the sulfonium salt quencher of 4.
6. The polymer of 5, further comprising repeat units having the formula (b1) or (b2):

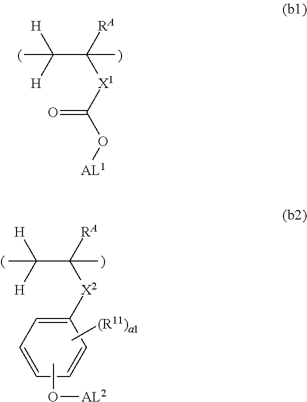

wherein R^{A} is each independently hydrogen, fluorine, methyl group or trifluoromethyl group,
   X¹ is a single bond, phenylene group, naphthylene group, *—C(═O)—O—X¹¹— or *—C(═O)—NH—X¹¹—, the phenylene group or naphthylene group may be substituted with hydroxy group, nitro group, cyano group, a C₁-C₁₀ saturated hydrocarbyl group which may contain fluorine, a C₁-C₁₀ saturated hydrocarbyloxy group which may contain fluorine, or halogen, X¹¹ is a C₁-C₁₀ saturated hydrocarbylene group, phenylene group, or naphthylene group, the saturated hydrocarbylene group may contain hydroxy group, ether bond, ester bond or lactone ring,
   X² is a single bond, *—C(═O)—O— or *—C(═O)—NH—,
   * designates a point of attachment to the carbon atom in the backbone,
   R¹¹ is halogen, cyano group, hydroxy group, nitro group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, a plurality of R¹¹ may be identical or different when a1 is 2 or more,
   AL¹ and AL² are each independently an acid labile group, and
   a1 is 0, 1, 2, 3 or 4.
7. The polymer of 5 or 6, further comprising repeat units having the formula (b3):

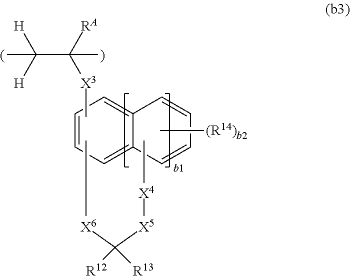

wherein b1 is 0 or 1, b2 is 0, 1, 2 or 3 when b1 is 0, b2 is 0, 1, 2, 3, 4 or 5 when b1 is 1,
   R^{A} is hydrogen, fluorine, methyl group or trifluoromethyl group,
   X³ is a single bond, *—C(═O)—O— or *—C(═O)—NH—, * designates a point of attachment to the carbon atom in the backbone,
   R¹² and R¹³ are each independently hydrogen or a C₁-C₂₀ hydrocarbyl group which may contain a heteroatom, R¹² and R¹³ may bond together to form a ring with the carbon atoms to which they are attached,
   R¹⁴ is halogen, hydroxy group, cyano group, nitro group, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{14A})(R^{14B}), R^{14A} and R^{14B} are each independently hydrogen or a C₁-C₆ hydrocarbyl group, a plurality of R¹⁴ may be identical or different and a plurality of R¹⁴ may bond together to form a ring with the carbon atoms to which they are attached, when b2 is 2 or more,
   X⁴ is a single bond, C₁-C₄ aliphatic hydrocarbylene group, carbonyl group, sulfonyl group or a group obtained by combining the foregoing, and
   X⁵ and X⁶ are each independently oxygen or sulfur, provided that X⁴ and X⁶ are bonded to adjacent carbon atoms on the aromatic ring.
8. The polymer of any one of 5 to 7, further comprising repeat units having the formula (c):

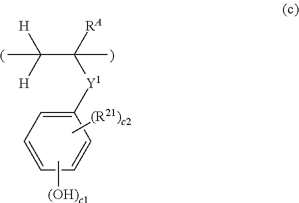

wherein R^{A} is hydrogen, fluorine, methyl group, or trifluoromethyl group,
   Y¹ is a single bond, *—C(═O)—O— or *—C(═O)—NH—, * designates a point of attachment to the carbon atom in the backbone,
   R²¹ is halogen, nitro group, cyano group, carboxy group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom,
   c1 is 1, 2, 3 or 4, and c2 is 0, 1, 2 or 3, provided that c1+c2 is from 1 to 5.
9. The polymer of any one of 5 to 8, further comprising repeat units derived from an onium salt monomer containing a fluorosulfonate anion having a polymerizable group and at least one iodine, and a sulfonium cation.
10. The polymer of any one of 5 to 9, further comprising repeat units having the formula (e):

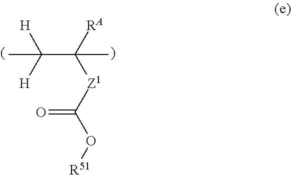

wherein R^{A} is hydrogen, fluorine, methyl group, or trifluoromethyl group,
   Z¹ is a single bond, phenylene group, naphthylene group, *—C(═O)—O—Z¹¹- or *—C(═O)—NH—Z¹¹—, the phenylene group or naphthylene group may be substituted with hydroxy group, nitro group, cyano group, a C₁-C₁₀ saturated hydrocarbyl group which may contain fluorine, a C₁-C₁₀ saturated hydrocarbyloxy group which may contain fluorine, or halogen, * designates a point of attachment to the carbon atom in the backbone, Z¹¹ is a C₁-C₁₀ saturated hydrocarbylene group, phenylene group or naphthylene group, the saturated hydrocarbylene group may contain hydroxy group, ether bond, ester bond or lactone ring, and
   R⁵¹ is hydrogen, or a C₁-C₂₀ group containing at least one structure selected from a hydroxy group exclusive of phenolic hydroxy group, a cyano group, carbonyl group, carboxy group, ether bond, ester bond, sulfonic ester bond, carbonate bond, lactone ring, sultone ring, and carboxylic anhydride (—C(═O)—O—C(═O)—).
11. A chemically amplified resist composition comprising (A) a base polymer containing the polymer of any one of 5 to 10.
12. The chemically amplified resist composition of 11, further comprising (B) an organic solvent.
13. The chemically amplified resist composition of 11 or 12, further comprising (C) a quencher.
14. The chemically amplified resist composition of any one of 11 to 13, further comprising (D) an acid generator.
15. The chemically amplified resist composition of any one of 11 to 14, further comprising (E) a surfactant.
16. A pattern forming process comprising the steps of applying the chemically amplified resist composition of any one of 11 to 15 onto a substrate to form a resist film thereon, exposing the resist film to high-energy radiation, and developing the exposed resist film in a developer.
17. The pattern forming process of 16, wherein the high-energy radiation is ArF excimer laser having a wavelength 193 nm, KrF excimer laser having a wavelength 248 nm, EB, or EUV having a wavelength 3 to 15 nm.

### Advantageous Effects of the Invention

A resist film containing a polymer comprising repeat units derived from a sulfonium salt monomer having formula (a) has good solvent solubility, and a large amount of iodine atoms, and is characterized by a short distance of acid diffusion. Accordingly, a reduction of resolution due to blur by acid diffusion can be prevented, and LWR and CDU can be improved. Iodine, which is highly absorptive to EUV having a wavelength 13.5 nm, generates secondary electrons upon exposure, so that the sensitivity is increased. Further, a —SF₅ group contained in the cation is a functional group that promotes solvent solubility, as well as a strong electron-withdrawing group, which lowers LUMO of the frontier molecular orbit theory by bonding to the aromatic ring of a triaryl sulfonium cation, so that the cation is more likely to accept secondary electrons, whereby decomposition of the sulfonium cation is accelerated. On the other hand, a carboxylate anion is relatively highly basic, so that an acid generated from a strong acid can be effectively trapped. Accordingly, it is possible to construct a chemically amplified resist composition having a high sensitivity and improved LWR and CDU. The aromatic ring acts as a group exhibiting good etch resistance, and is suitable for fine pattern formation.

### DESCRIPTION OF THE PREFERRED EMBODIMENT

### Sulfonium Salt Monomer

The inventive sulfonium salt monomer has the formula (a).

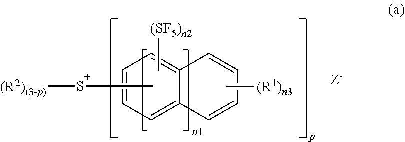

In formula (a), p is 1, 2 or 3.

In formula (a), n1 is 0 or 1. The sulfonium salt monomer has a benzene ring when n1 is 0, and a naphthalene ring when n1 is 1, and n1 is preferably 0 from the aspect of solvent solubility. n2 is 1 or 2, From the aspect of reactant availability, n2 is preferably 1. n3 is 0, 1, 2 or 3, From the aspect of reactant availability, n3 is preferably 0, 1 or 2. n2+n3 is from 1 to 5 when n1 is 0, n2+n3 is from 1 to 7 when n1 is 1,

In formula (a), R¹ is halogen, nitro group, cyano group, hydroxy group, a C₁-C₂₀ hydrocarbyl group which may contain a heteroatom, a C₁-C₂₀ hydrocarbyloxy group which may contain a heteroatom, a C₁-C₂₀ hydrocarbyloxy group, or a C₁-C₂₀ hydrocarbylthio group which may contain a heteroatom. Examples of the halogen include fluorine, chlorine, bromine and iodine atoms. The hydrocarbyl group and hydrocarbyl moiety of the hydrocarbyloxy and hydrocarbylthio groups may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl and icocyl groups; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl and adamantyl groups; C₂-C₂₀ alkenyl groups such as vinyl, allyl, propenyl, butenyl and hexenyl groups; C₃-C₂₀ cyclic unsaturated aliphatic hydrocarbyl groups such as a cyclohexenyl group; C₆-C₂₀ aryl groups such as phenyl and naphthyl groups; C₇-C₂₀ aralkyl groups such as benzyl, 1-phenylethyl and 2-phenylethyl groups; and combinations thereof. Inter alia, aryl groups are preferred. Some or all of hydrogen atoms of the hydrocarbyl group may be replaced by a group containing a heteroatom such as oxygen, sulfur, nitrogen or halogen, some constituent —CH₂— of the hydrocarbyl group may be replaced by a group containing a heteroatom such as oxygen, sulfur or nitrogen, and as a result, the hydrocarbyl group may contain a hydroxy group, a cyano group, fluorine, chlorine, bromine, iodine, a carbonyl group, an ether bond, an ester bond, a sulfonic ester bond, a carbonate bond, a lactone ring, a sultone ring, carboxylic anhydride (—C(═O)—O—C(═O)—), a haloalkyl group, or the like. A plurality of R¹ may be identical or different when n3 is 2 or 3. A plurality of R¹ may bond together to form a ring with the carbon atoms to which they are attached when n3 is 2 or 3. Of the rings, 5 to 8-membered rings are preferred.

In formula (a), R² are each independently halogen or a C₁-C₃₀ hydrocarbyl group which may contain a heteroatom.

Examples of the halogen represented by R² include fluorine, chlorine, bromine, and iodine.

The hydrocarbyl group represented by R² may be saturated or unsaturated, and may be straight, branched, or cyclic. Examples thereof include C₁-C₃₀ alkyl groups such as methyl, ethyl, n-propyl, isopropyl, n-butyl, sec-butyl and tert-butyl groups; C₃-C₃₀ cyclic saturated hydrocarbyl groups such as cyclopropyl, cyclopentyl, cyclohexyl, cyclopropylmethyl, 4-methylcyclohexyl, cyclohexylmethyl, norbornyl and adamantyl groups; C₂-C₃₀ alkenyl groups such as vinyl, allyl, propenyl, butenyl and hexenyl groups; C₃-C₃₀ cyclic unsaturated aliphatic hydrocarbyl groups such as a cyclohexenyl group; C₆-C₃₀ aryl groups such as phenyl, naphthyl and thienyl groups; C₇-C₃₀ aralkyl groups such as benzyl, 1-phenylethyl and 2-phenylethyl groups; and combinations thereof. The aryl groups are preferred. In the hydrocarbyl group, some or all hydrogen atoms may be substituted by a moiety containing a heteroatom such as oxygen, sulfur, nitrogen or halogen and some constituent —CH₂— may be replaced by a moiety containing a heteroatom such as oxygen, sulfur or nitrogen, so that the group may contain hydroxy, fluorine, chlorine, bromine, iodine, cyano, nitro, carbonyl, ether bond, ester bond, sulfonic ester bond, carbonate bond, lactone ring, sultone ring, carboxylic anhydride (—C(═O)—O—C(═O)—), or haloalkyl moiety.

Two of three substituents bonded to S⁺ may bond together to form a ring with a sulfur atom to which they are attached, and Examples of the structure of the ring include those represented by the following formula.

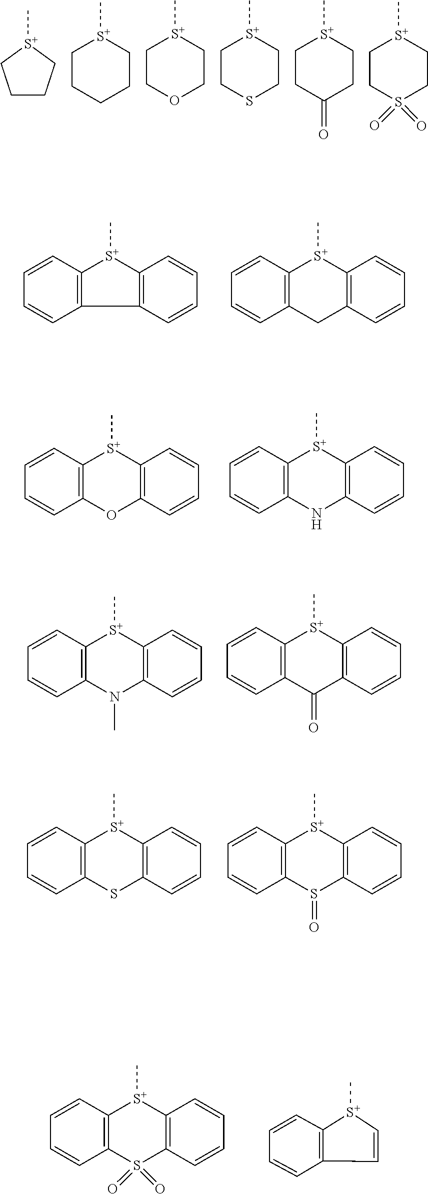

Herein the broken line denotes another point of attachment in the sulfonium cation.

Of the sulfonium salt monomers of formula (a), a structure having the formula (a1) is preferred.

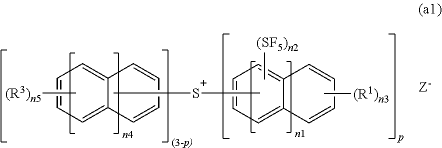

wherein p, n1 to n3, and R¹ and Z⁻ are as defined above,

In formula (a1), n4 is 0 or 1. The sulfonium salt monomer has a benzene ring when n4 is 0, and a naphthalene ring when n4 is 1, and n4 is preferably 0 from the aspect of solvent solubility. The subscript n5 is 0, 1, 2, 3, 4 or 5, and n5 is preferably 0, 1 or 2 from the aspect of reactant availability.

In formula (a1), R³ is halogen, nitro group, cyano group, hydroxy group, a C₁-C₂₀ hydrocarbyl group which may contain a heteroatom, a C₁-C₂₀ hydrocarbyloxy group which may contain a heteroatom, a C₁-C₂₀ hydrocarbyloxy group, or a C₁-C₂₀ hydrocarbylthio group which may contain a heteroatom. Examples thereof are as exemplified above as a group R¹, but not limited thereto.

Examples of the cation in the sulfonium salt monomer having formula (a) are shown below, but not limited thereto.

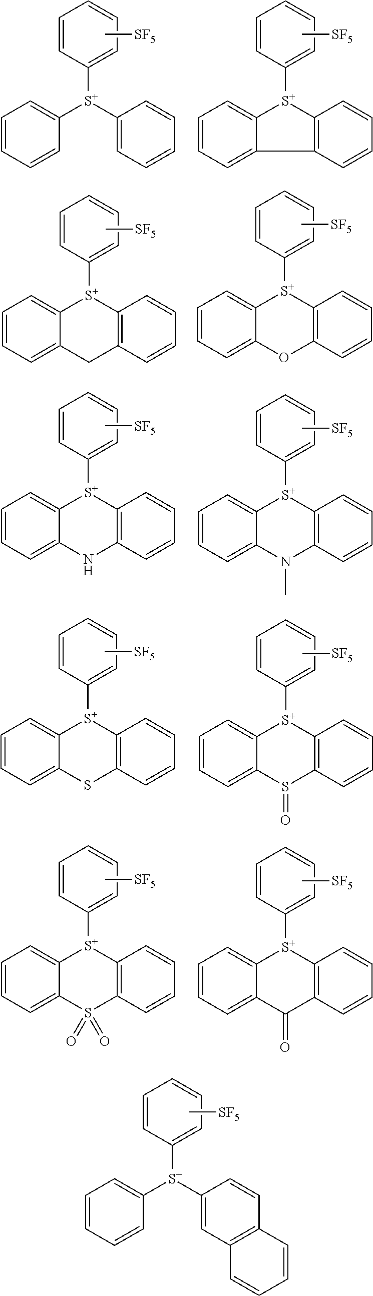

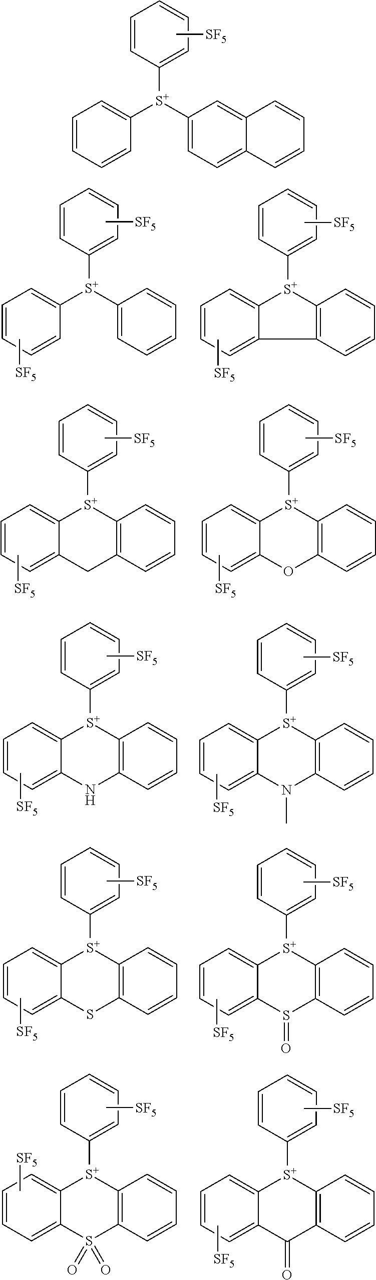

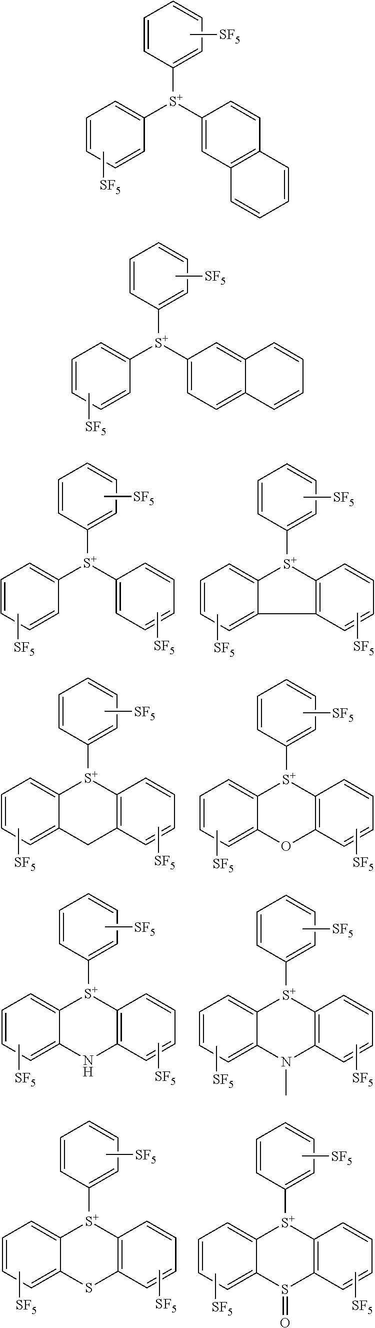

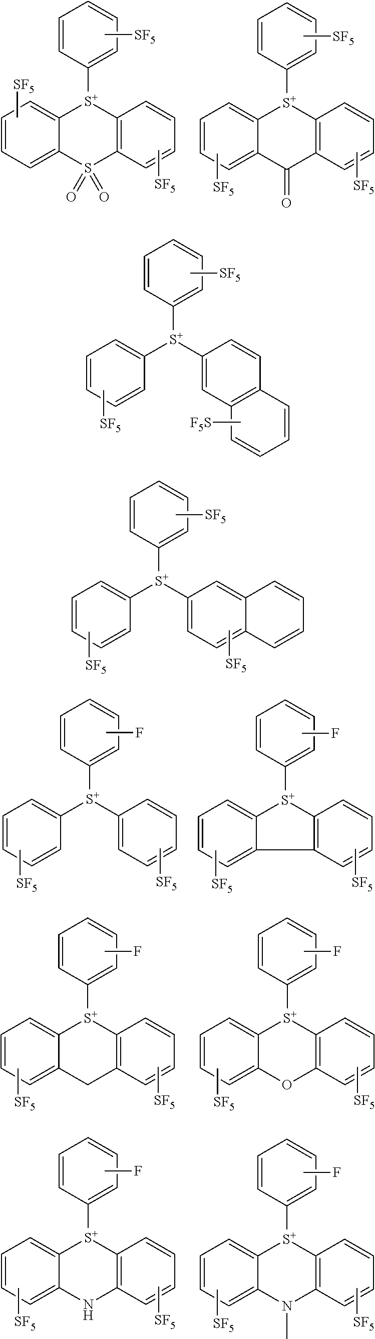

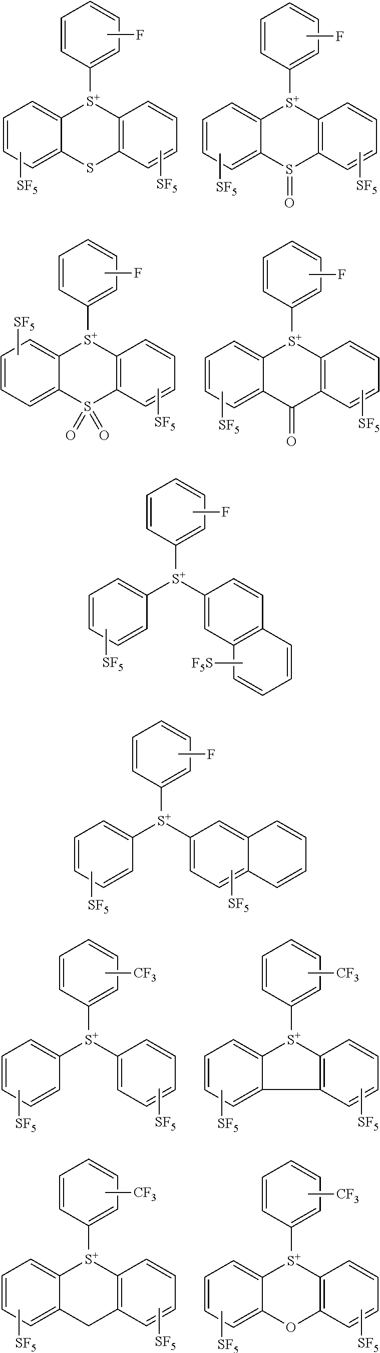

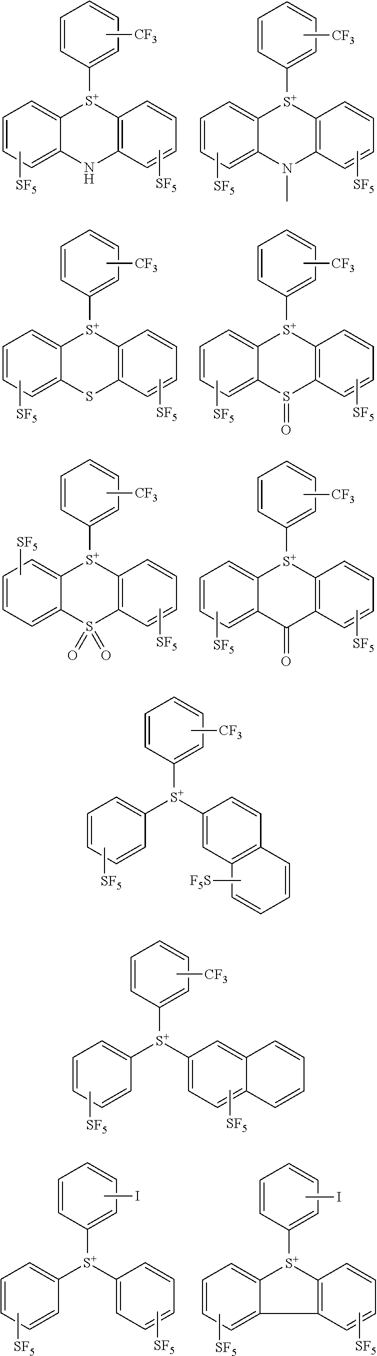

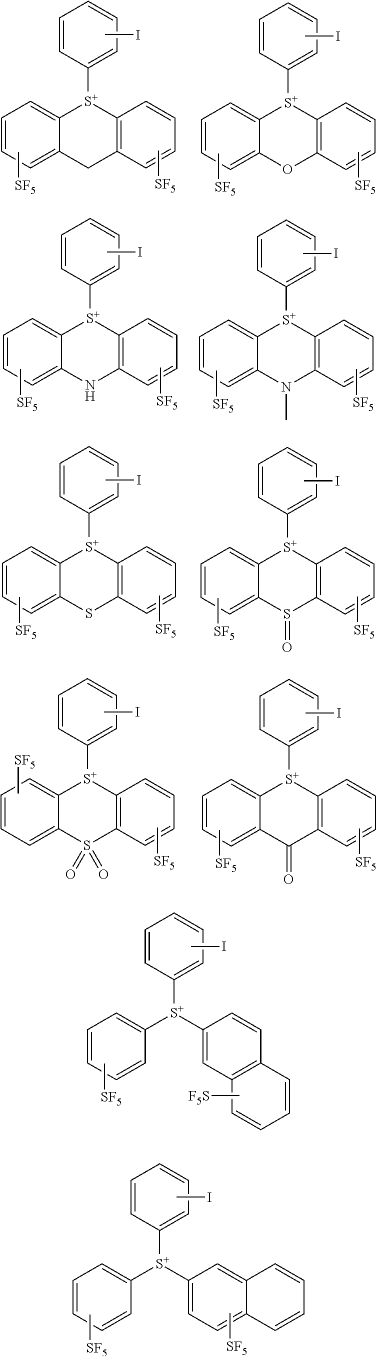

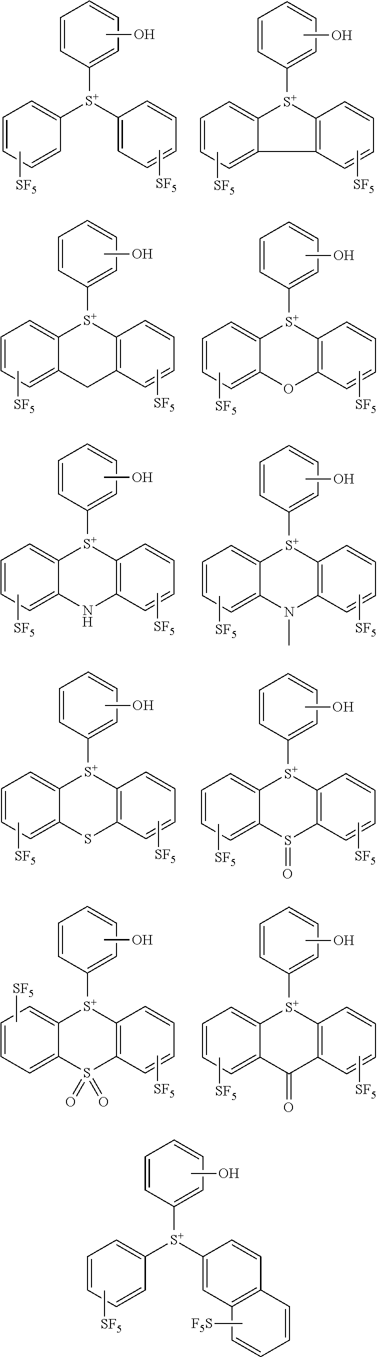

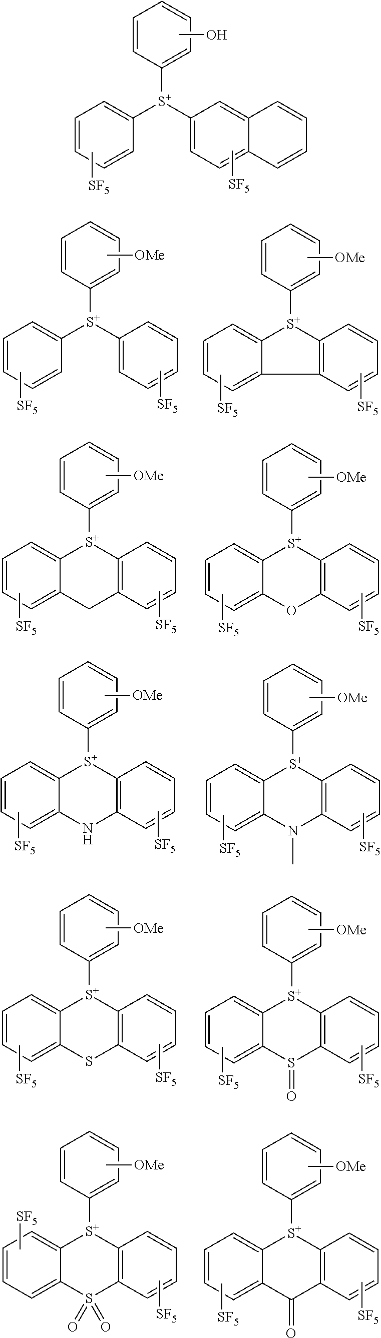

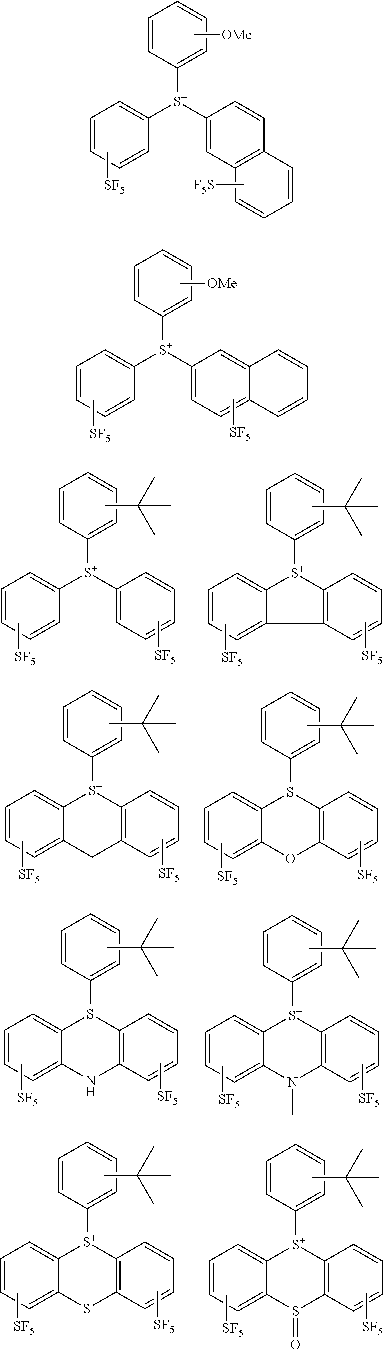

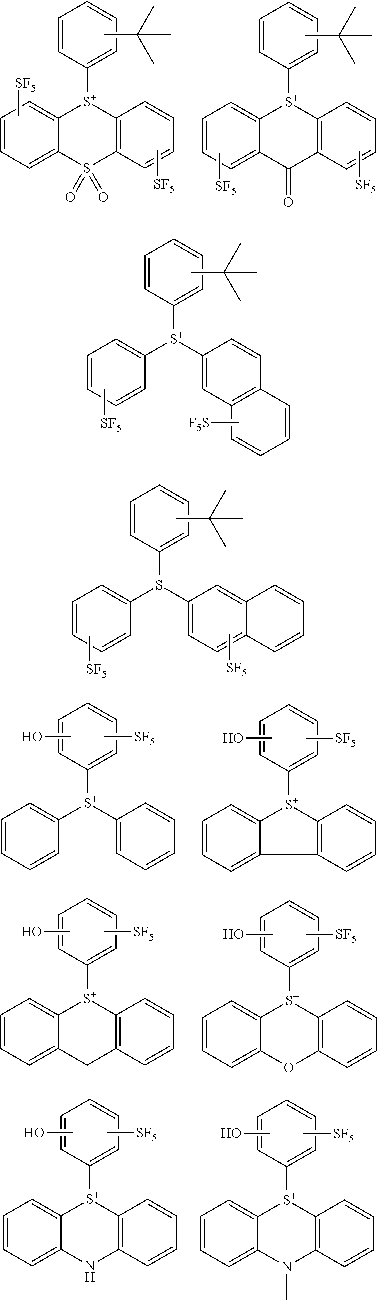

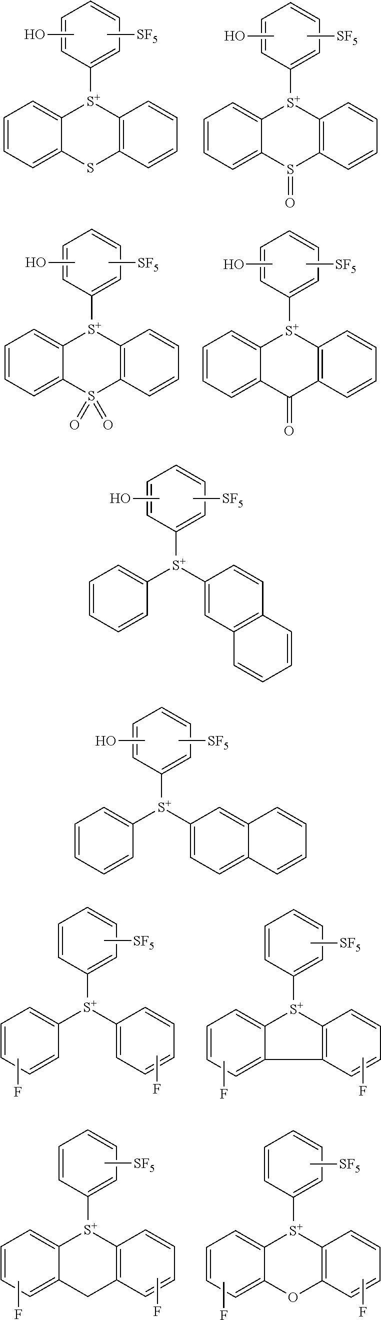

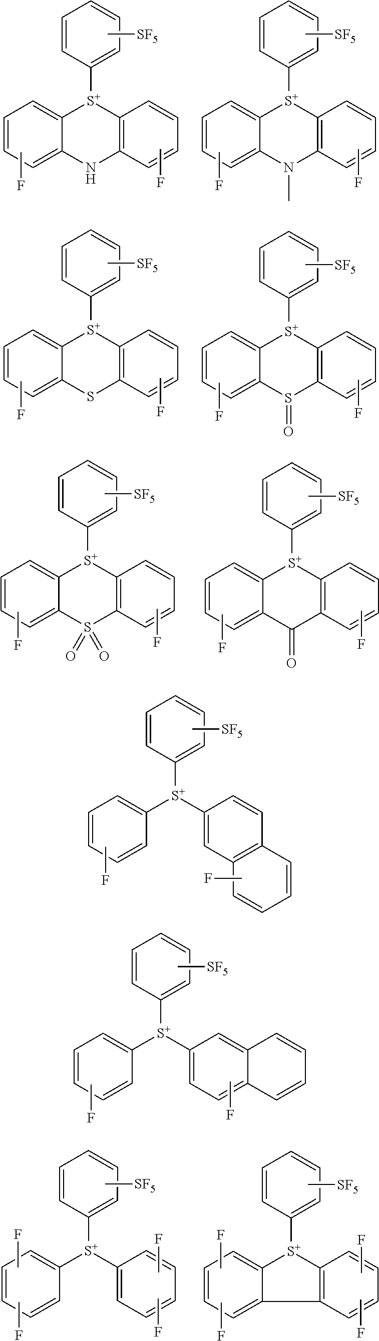

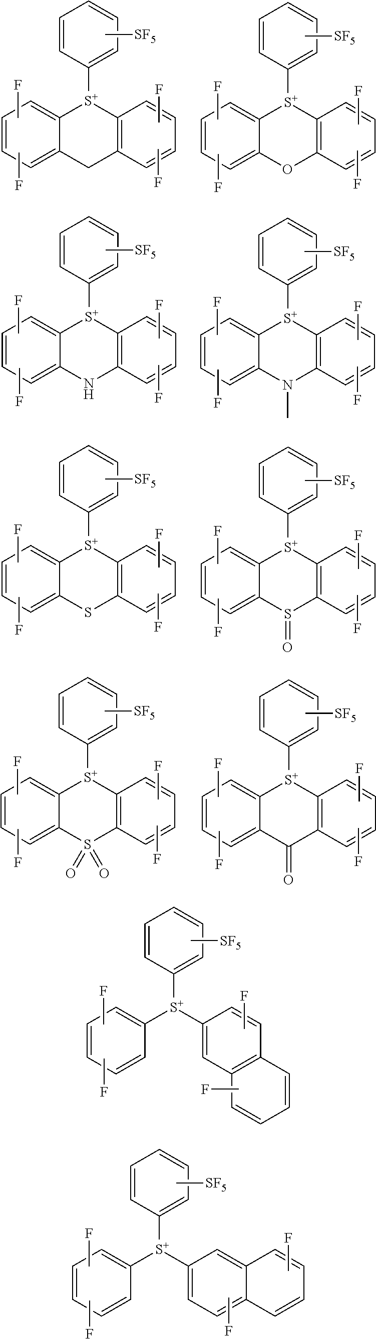

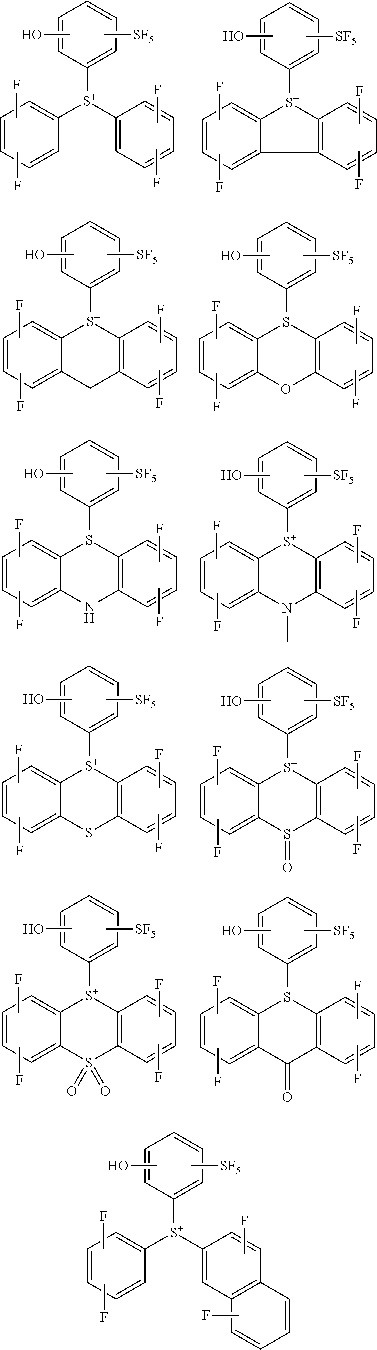

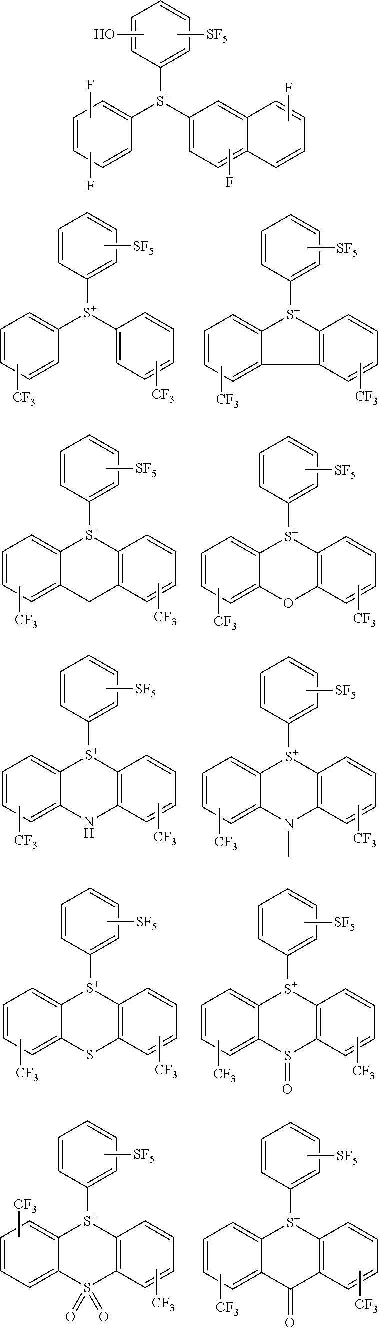

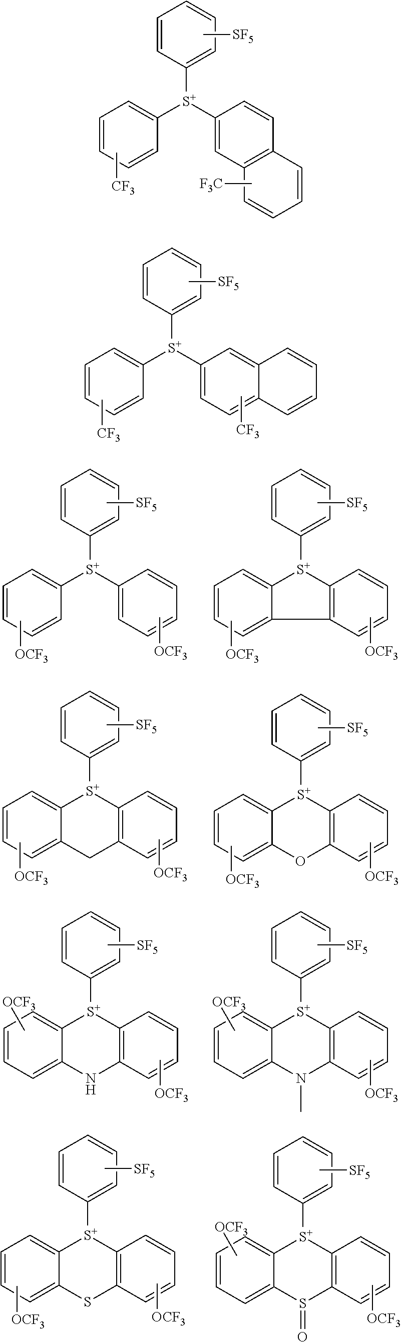

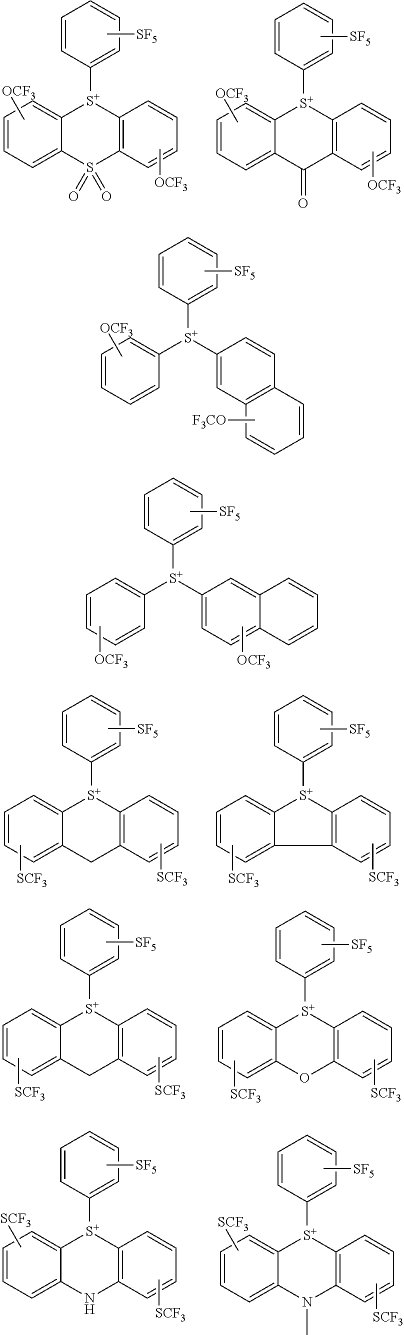

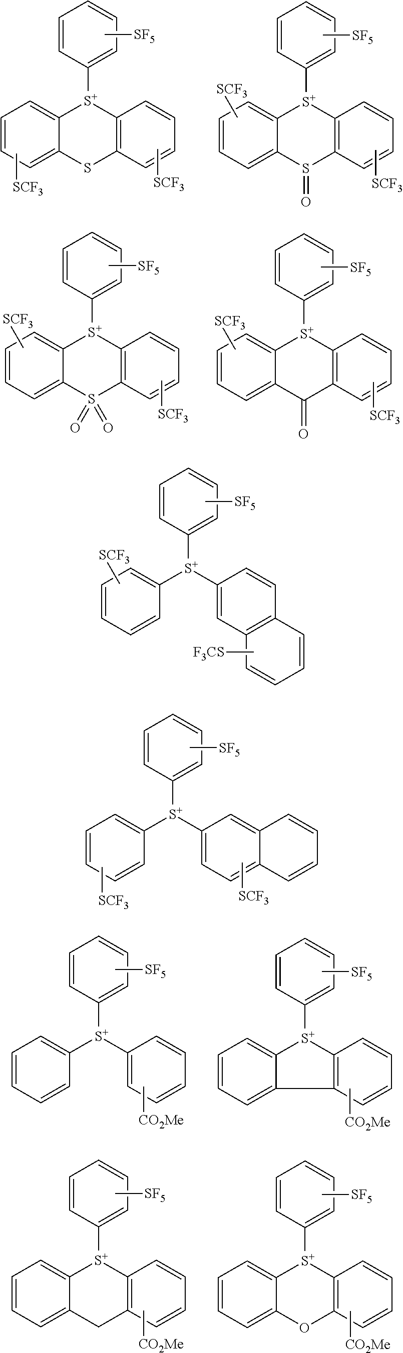

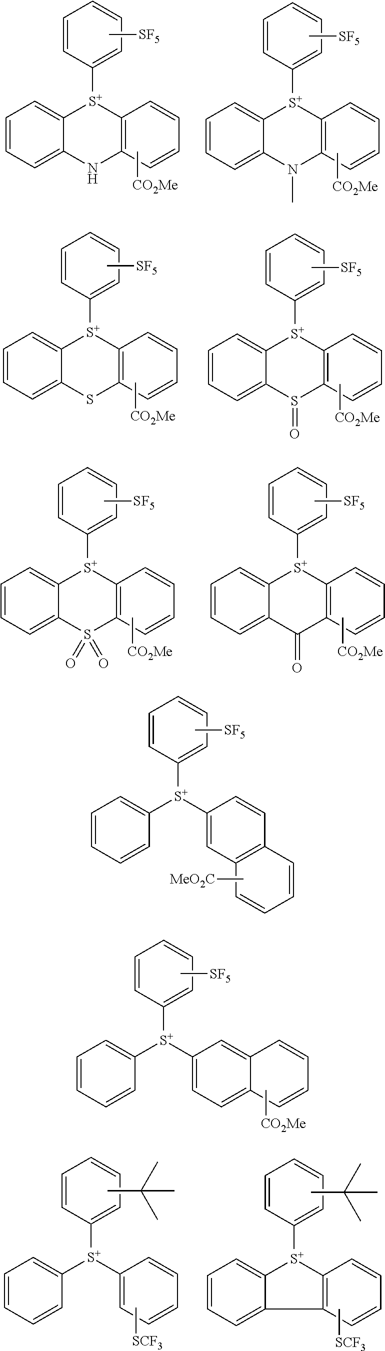

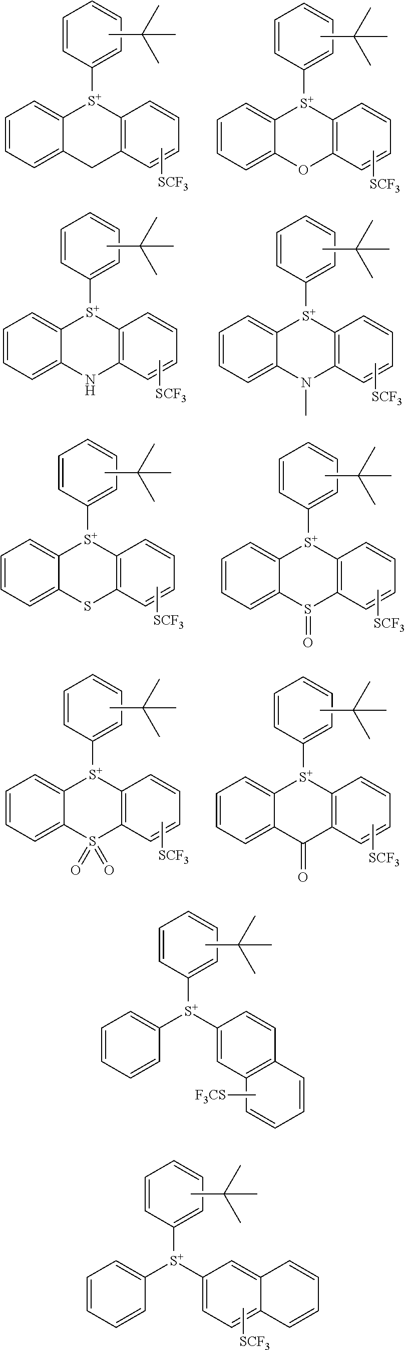

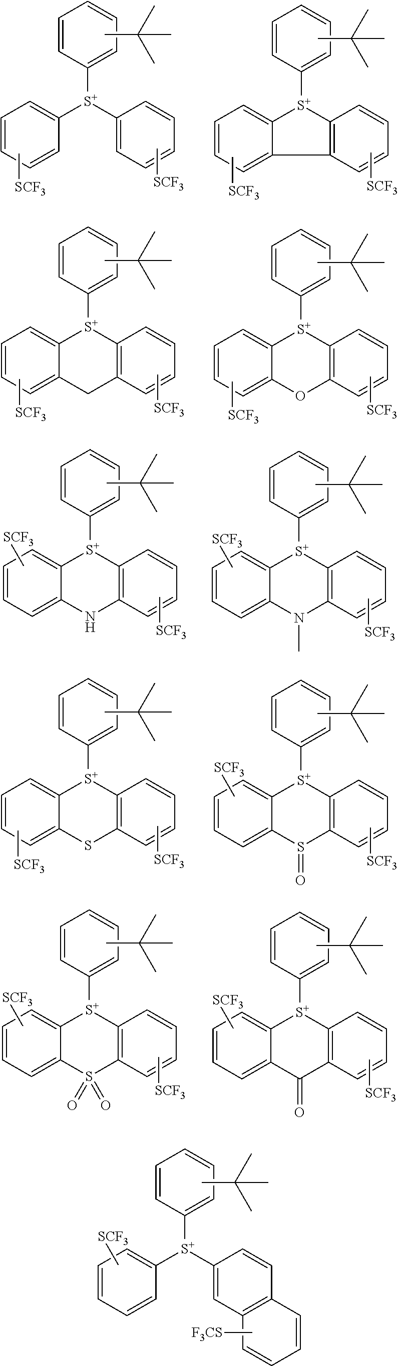

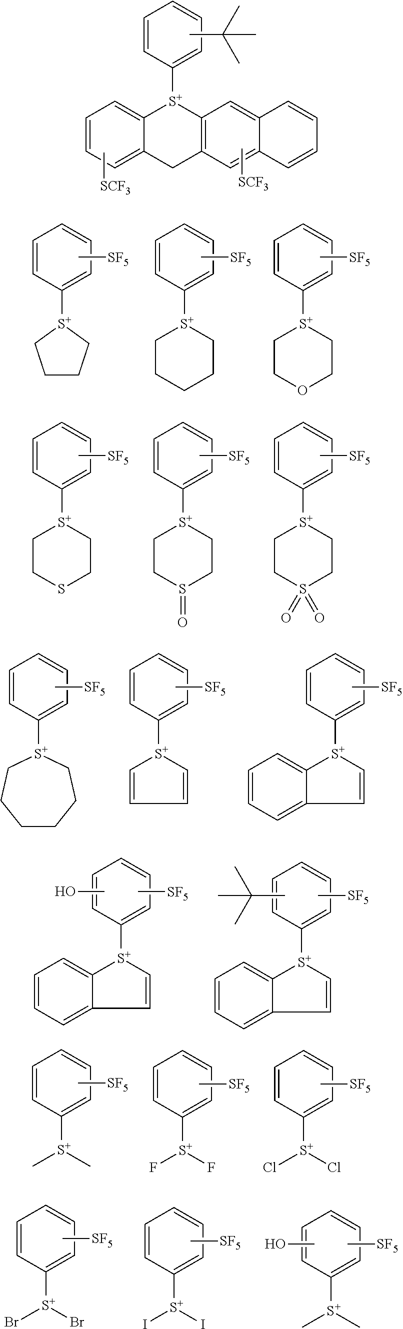

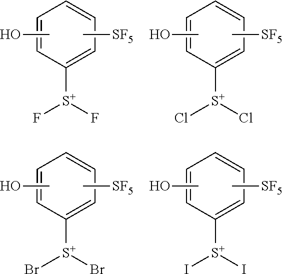

In formulae (a) and (a1), Z⁻ is a carboxylate anion having an aromatic vinyl structure and iodine. Of the carboxylate anions, a structure having the formula (Z) is preferred.

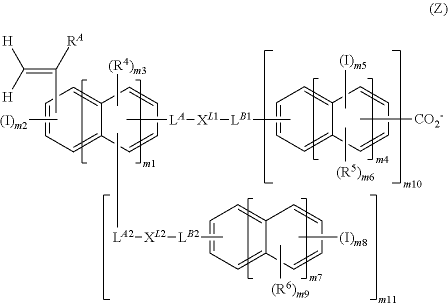

In formula (Z), m1 is 0 or 1. The sulfonium salt monomer has a benzene ring when m1 is 0, and a naphthalene ring when m1 is 1, and m1 is preferably 0 from the aspect of solvent solubility. m2 is 0, 1, 2, 3 or 4, From the aspect of reactant availability, m2 is preferably 0, 1, 2 or 3, more preferably 0, 1 or 2, still more preferably 0 or 1. m3 is 0, 1, 2 or 3,

In formula (Z), m4 is 0 or 1. The sulfonium salt monomer has a benzene ring when m4 is 0, and a naphthalene ring when m4 is 1, and m4 is preferably 0 from the aspect of solvent solubility. m5 is 0, 1, 2, 3 or 4, From the aspect of reactant availability, m5 is preferably 0, 1, 2 or 3, more preferably 0, 1 or 2. m6 is 0, 1, 2 or 3,

In formula (Z), m7 is 0 or 1. The sulfonium salt monomer has a benzene ring when m7 is 0, and a naphthalene ring when m7 is 1, and m7 is preferably 0 from the aspect of solvent solubility. m8 is 1, 2, 3 or 4, From the aspect of reactant availability, m8 is preferably 1, 2 or 3, more preferably 1 or 2. m9 is 0, 1, 2 or 3,

In formula (Z), m10 is 0 or 1. m11 is 0 or 1,

m2+m3+m11 is from 0 to 4 when m1 is 0, m2+m3+m11 is from 0 to 6 when m1 is 1, m5+m6 is from 0 to 4 when m4 is 0, m5+m6 is from 0 to 6 when m4 is 1, m8+m9 is from 0 to 5 when m7 is 0, m8+m9 is from 0 to 7 when m7 is 1, As the number of iodine atoms in the anion becomes larger, the amount of absorption of EUV increases, but precipitation in the resist composition may occur due to reduced solvent solubility. Therefore, m2+m5+m8 is preferably from 1 to 4.

In formula (Z), R^{A} is hydrogen, fluorine, methyl group, or trifluoromethyl group. Of these, hydrogen atom and methyl group are preferred, and hydrogen is more preferred.

In formula (Z), R⁴ is halogen exclusive of iodine, nitro group, hydroxy group, a C₁-C₂₀ hydrocarbyl group which may contain a heteroatom, a C₁-C₂₀ hydrocarbyloxy group which may contain a heteroatom, a C₁-C₂₀ hydrocarbyloxy group, or a C₁-C₂₀ hydrocarbylthio group which may contain a heteroatom. The halogen exclusive of iodine is preferably fluorine, chlorine, bromine or iodine, more preferably fluorine. The hydrocarbyl group and hydrocarbyl moiety of the hydrocarbyloxy and hydrocarbylthio groups may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl and icocyl groups; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl and adamantyl groups; C₂-C₂₀ alkenyl groups such as vinyl, allyl, propenyl, butenyl and hexenyl groups; C₃-C₂₀ cyclic unsaturated hydrocarbyl groups such as a cyclohexenyl group; C₆-C₂₀ aryl groups such as phenyl and naphthyl groups; C₇-C₂₀ aralkyl groups such as benzyl, 1-phenylethyl and 2-phenylethyl groups; and combinations thereof. Some or all of hydrogen atoms of the hydrocarbyl group may be replaced by a group containing a heteroatom such as oxygen, sulfur, nitrogen or halogen, some constituent —CH₂— of the hydrocarbyl group may be replaced by a group containing a heteroatom such as oxygen, sulfur or nitrogen, and as a result, the hydrocarbyl group may contain a hydroxy group, a cyano group, fluorine, chlorine, bromine, iodine, a carbonyl group, an ether bond, an ester bond, a sulfonic ester bond, a carbonate bond, a lactone ring, a sultone ring, carboxylic anhydride (—C(═O)—O—C(═O)—), a haloalkyl group, or the like. A plurality of R⁴ may be identical or different when m3 is 2 or 3.

Two R⁴ may bond together to form a ring with the aromatic ring carbon atom to which they are attached when m3 is 2 or 3. Examples of the ring formed herein include cyclopropane, cyclobutane, cyclopentane, cyclohexane, norbornane and adamantane rings. Some or all of hydrogen atoms in the ring may be replaced by a group containing a heteroatom such as oxygen, sulfur, nitrogen or halogen, some constituent —CH₂— in the ring may be replaced by a group containing a heteroatom such as oxygen, sulfur or nitrogen, and as a result, the hydrocarbyl group may contain a hydroxy group, fluorine, chlorine, bromine, iodine, a cyano group, a carbonyl group, an ether bond, an ester bond, a sulfonic ester bond, a carbonate bond, a lactone ring, a sultone ring, carboxylic anhydride (—C(═O)—O—C(═O)—), a haloalkyl group, or the like.

In formula (Z), R⁵ is halogen exclusive of iodine, nitro group, hydroxy group, a C₁-C₂₀ hydrocarbyl group which may contain a heteroatom, a C₁-C₂₀ hydrocarbyloxy group which may contain a heteroatom, a C₁-C₂₀ hydrocarbyloxy group, or a C₁-C₂₀ hydrocarbylthio group which may contain a heteroatom. Examples of the halogen exclusive of iodine include fluorine, chlorine, bromine, and iodine. The hydrocarbyl group and hydrocarbyl moiety of the hydrocarbyloxy and hydrocarbylthio groups may be saturated or unsaturated and straight, branched or cyclic. Examples of the hydrocarbyl group are as exemplified above as a hydrocarbyl group R⁴, but not limited thereto. A plurality of R⁵ may be identical or different when m6 is 2 or 3.

Two R⁵ may bond together to form a ring with the carbon atoms to which they are attached when m6 is 2. Of the rings, 5 to 8-membered rings are preferred.

In formula (Z), R⁶ is halogen exclusive of iodine, nitro group, hydroxy group, a C₁-C₂₀ hydrocarbyl group which may contain a heteroatom, a C₁-C₂₀ hydrocarbyloxy group which may contain a heteroatom, a C₁-C₂₀ hydrocarbyloxy group, or a C₁-C₂₀ hydrocarbylthio group which may contain a heteroatom. Examples of the halogen exclusive of iodine include fluorine, chlorine, bromine, and iodine. The hydrocarbyl group and hydrocarbyl moiety of the hydrocarbyloxy and hydrocarbylthio groups may be saturated or unsaturated and straight, branched or cyclic. Examples of the hydrocarbyl group are as exemplified above as a hydrocarbyl group R⁴, but not limited thereto. A plurality of R⁶ may be identical or different when m9 is 2 or 3.

Two R⁶ may bond together to form a ring with the carbon atoms to which they are attached when m9 is 2. Of the rings, 5 to 8-membered rings are preferred.

In formula (Z), L^{A1}, L^{A2} and L^{B1} and L^{B2} are each independently a single bond, ether bond, carbonyl group, ester bond, sulfonic ester bond, amide bond, sulfonic amide bond, carbonate bond or carbamate bond. Inter alia, L^{A1} is preferably a single bond, ether bond, ester bond or sulfonic ester bond, more preferably an ether bond, ester bond or sulfonic ester bond. L^{A2} is preferably a single bond, ether bond, ester bond, amide bond, sulfonic amide bond or sulfonic ester bond, more preferably an ester bond or sulfonic ester bond. L^{B1} is preferably a single bond, ether bond, ester bond, amide bond or sulfonic ester bond, more preferably a single bond, ether bond or ester bond. L^{B2} is preferably a single bond, ether bond, ester bond, amide bond or sulfonic ester bond, more preferably a single bond, ether bond or ester bond.

When m11 is 1, L^{A1} and L^{A2} are preferably attached to adjacent carbon atoms on the aromatic ring. Here, the substituent containing a fluorosulfonate anion structure and the substituent containing an aromatic ring substituted with iodine are positioned spatially closer to each other, and thus, the sensitivity is expected to be higher.

In formula (Z), X^{L1} and X^{L2} are each independently a single bond, or a C₁-C₄₀ hydrocarbylene group which may contain a heteroatom. The hydrocarbylene group may be straight, branched or cyclic, and examples thereof include alkanediyl, cyclic saturated hydrocarbylene, and arylene groups. Examples of the heteroatom include oxygen, nitrogen and sulfur atoms.

Examples of the C₁-C₄₀ hydrocarbylene groups X^{L1} and X^{L2} which may contain a heteroatom are shown below, but not limited thereto. In the following formulae, * is a point of attachment to L^{A1} and L^{B1}, or L^{A2} and L^{B2}.

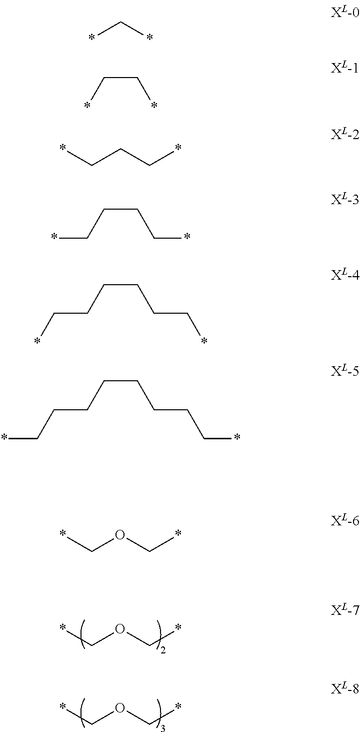

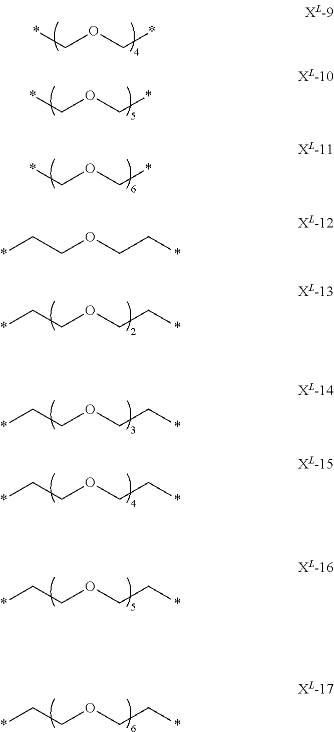

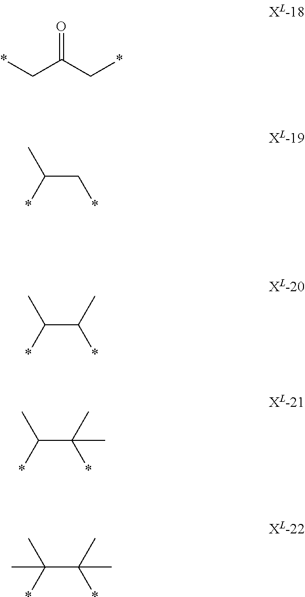

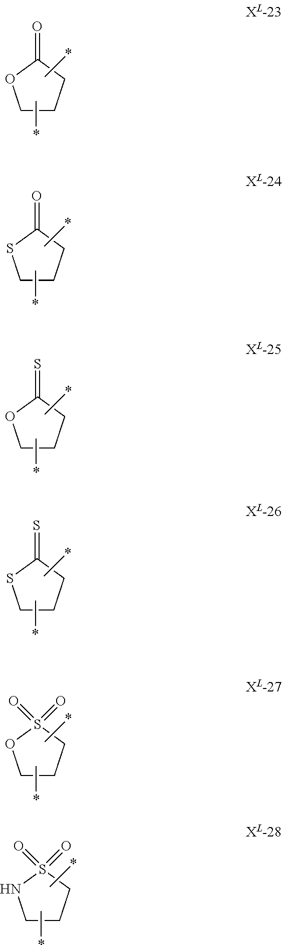

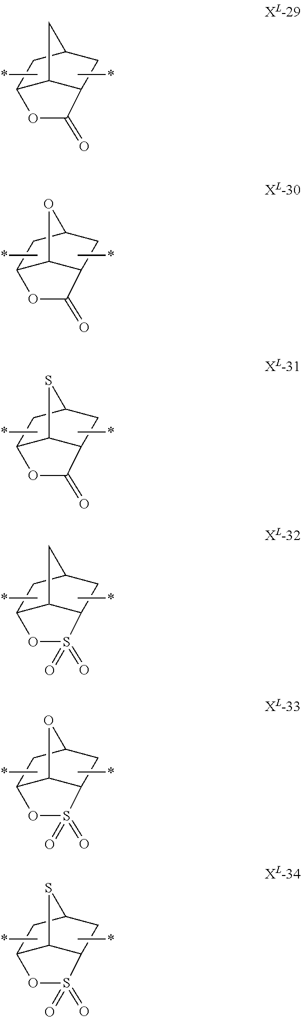

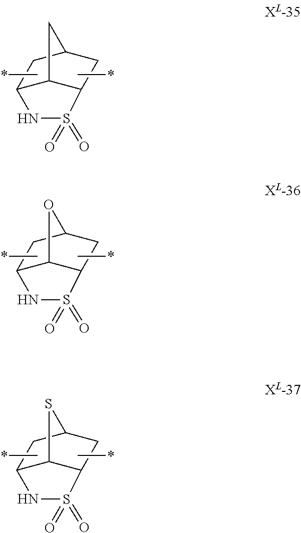

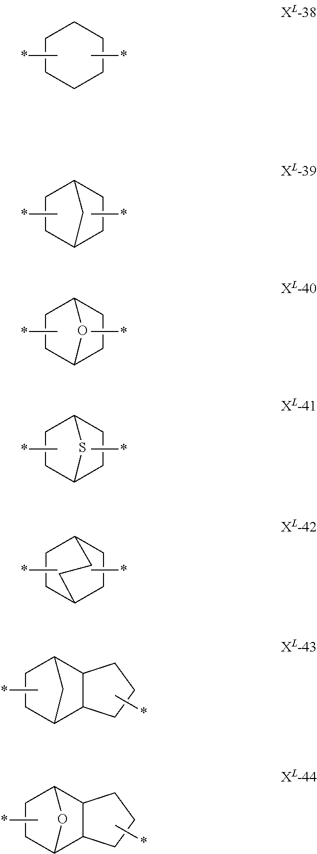

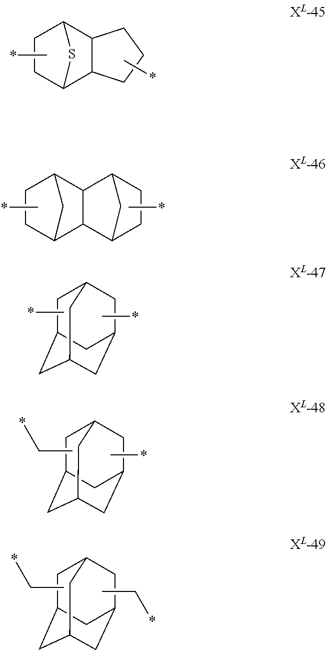

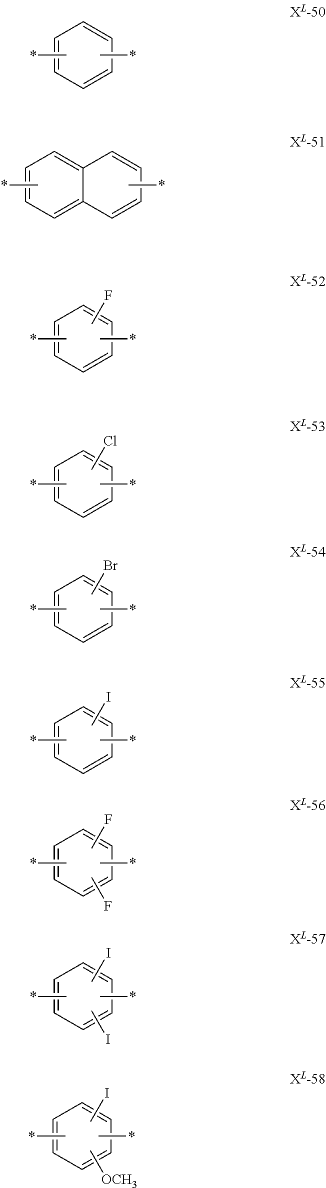

Of these, X^{L}-0 to X^{L}-22, X^{L}-29 to X^{L}-34, and X^{L}-47 to X^{L}-58 are preferred.

Examples of the anion in the onium salt monomer having formula (a) are shown below, but not limited thereto. In the following formulae, R^{A} is as defined above, and Me is a methyl group. The bonding positions of the substituents on the aromatic ring may be interchanged on the aromatic ring.

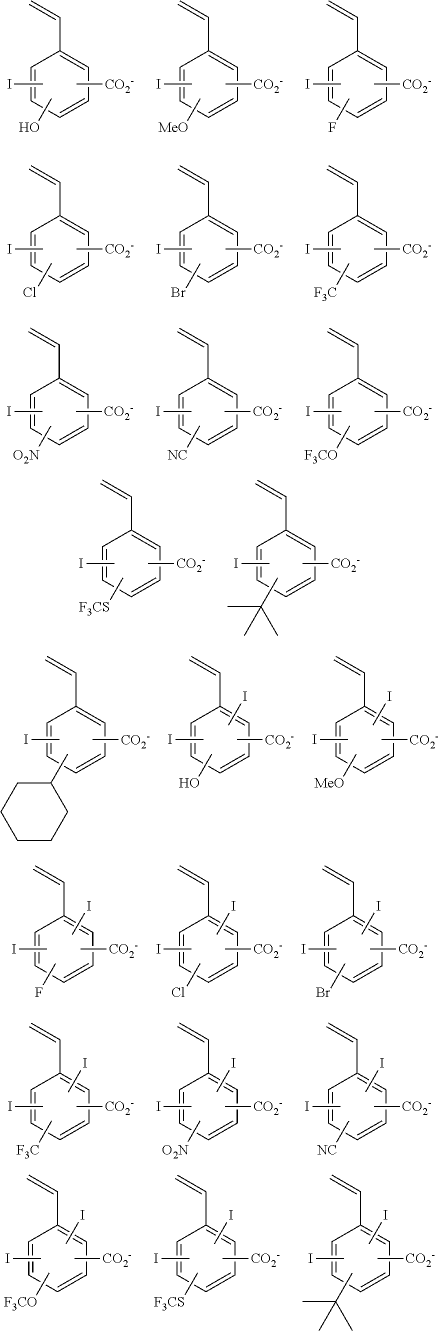

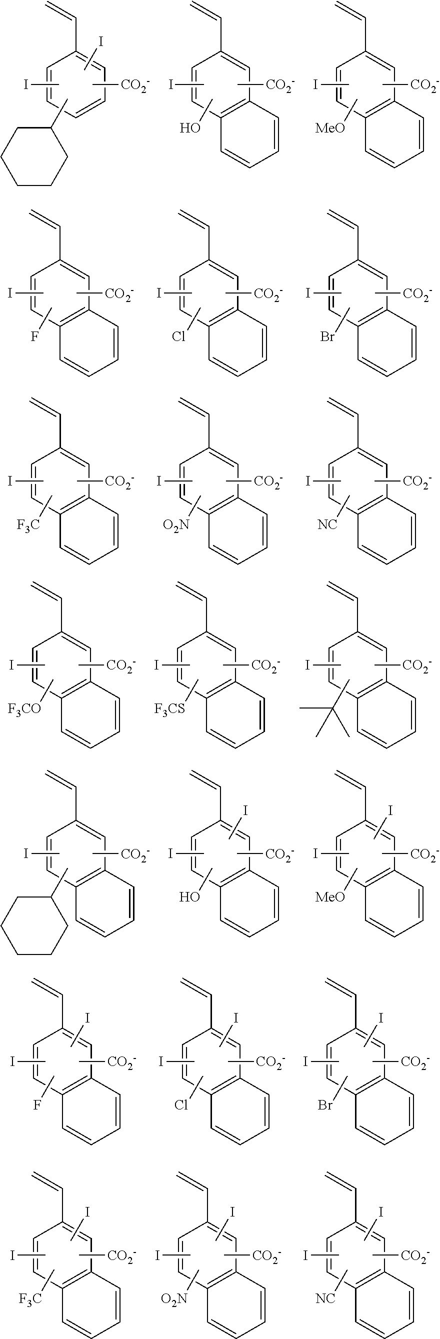

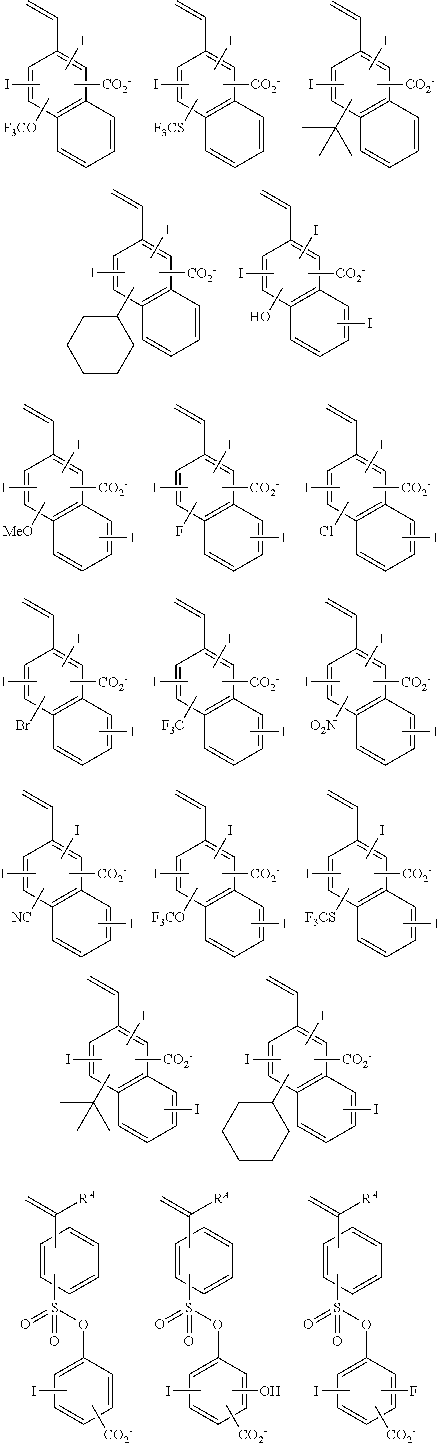

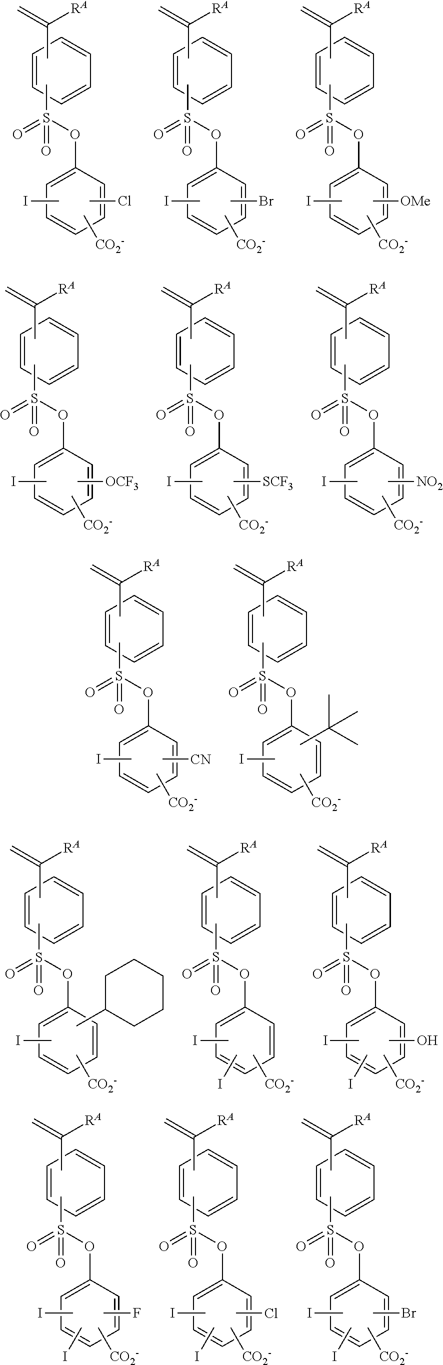

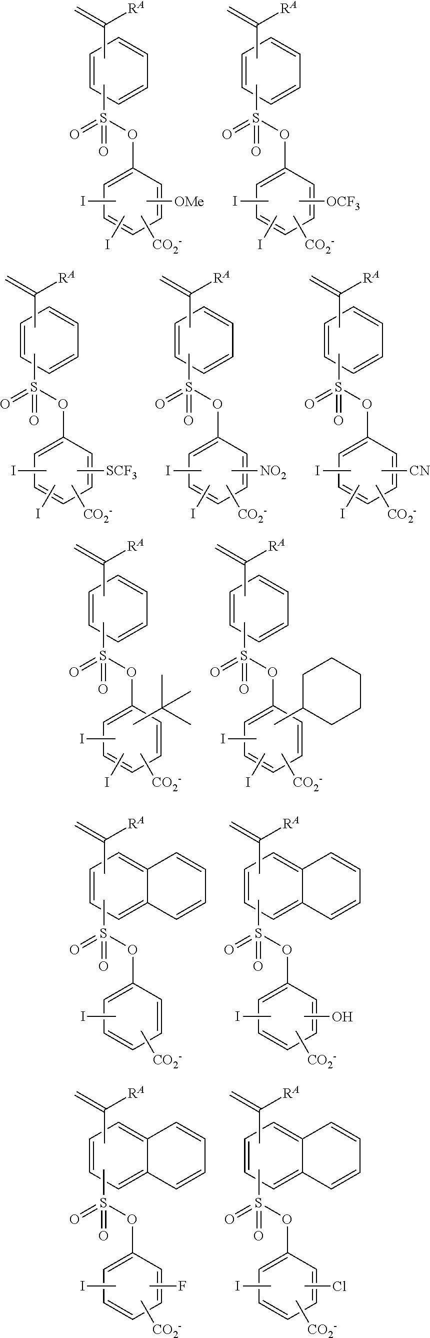

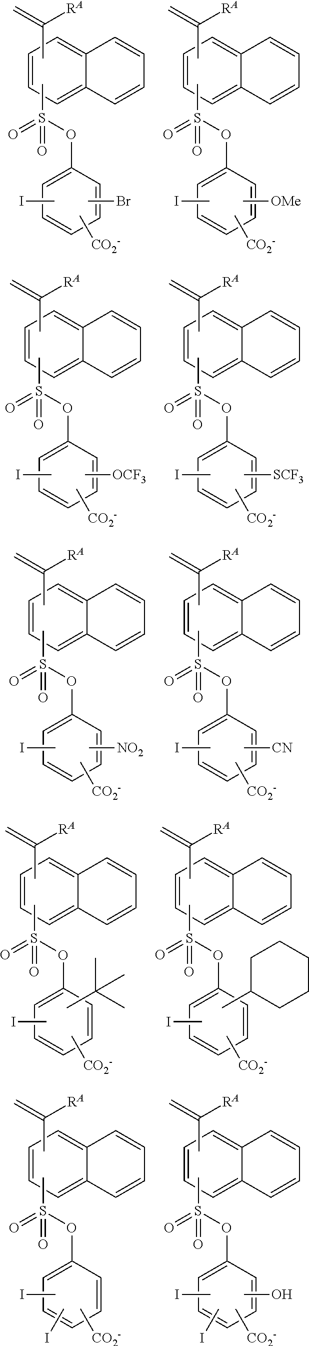

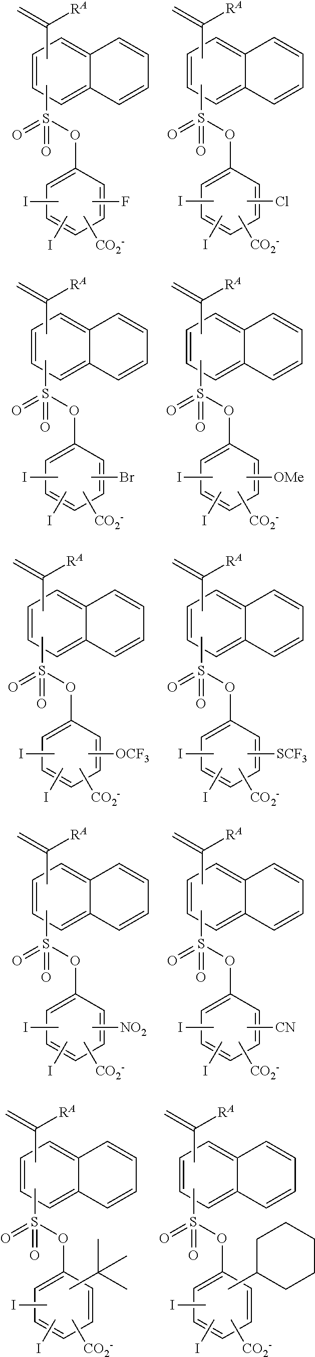

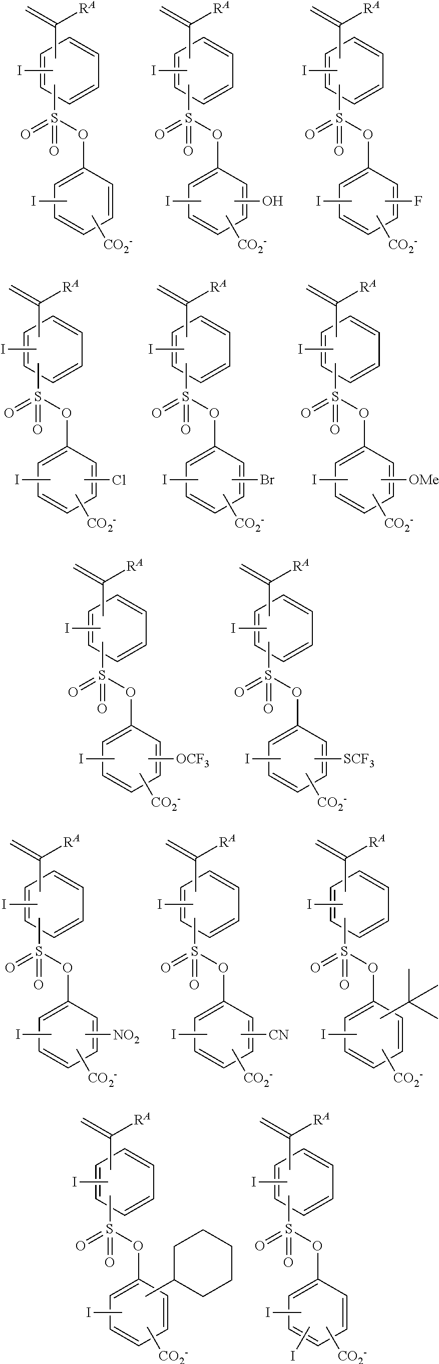

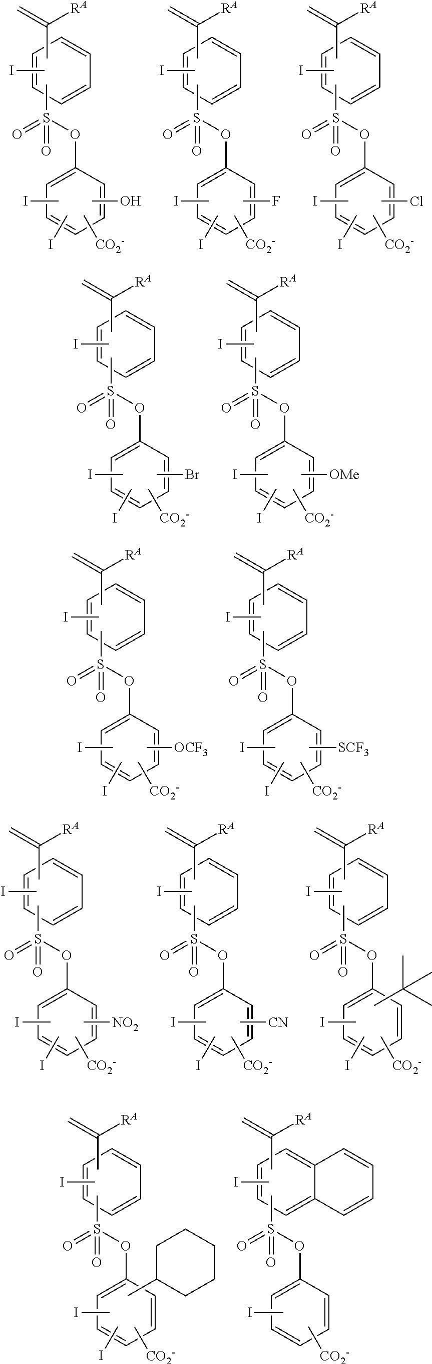

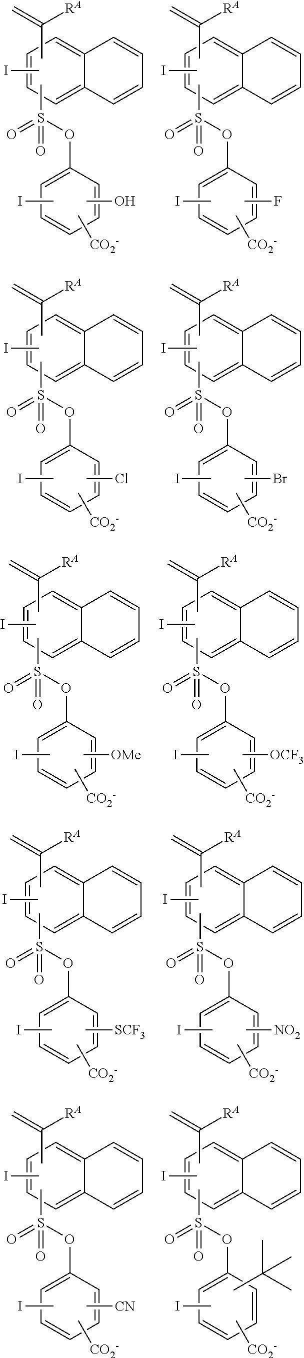

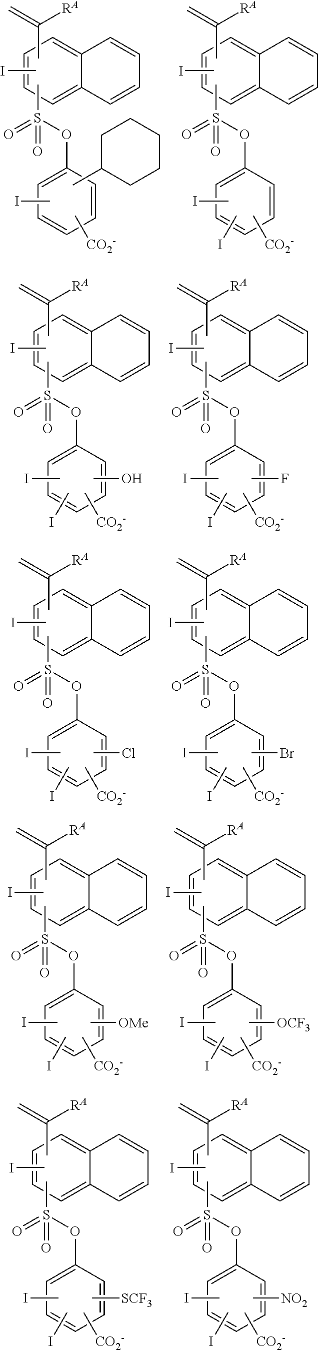

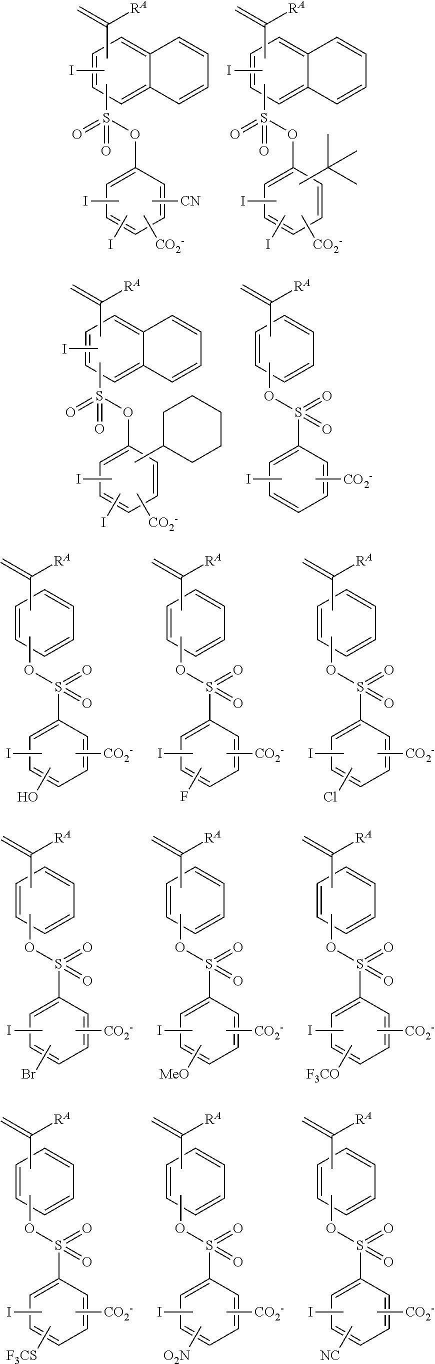

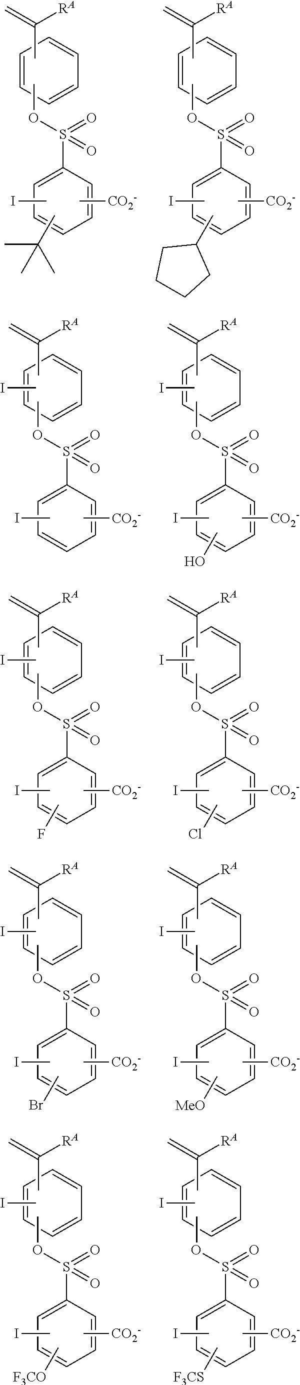

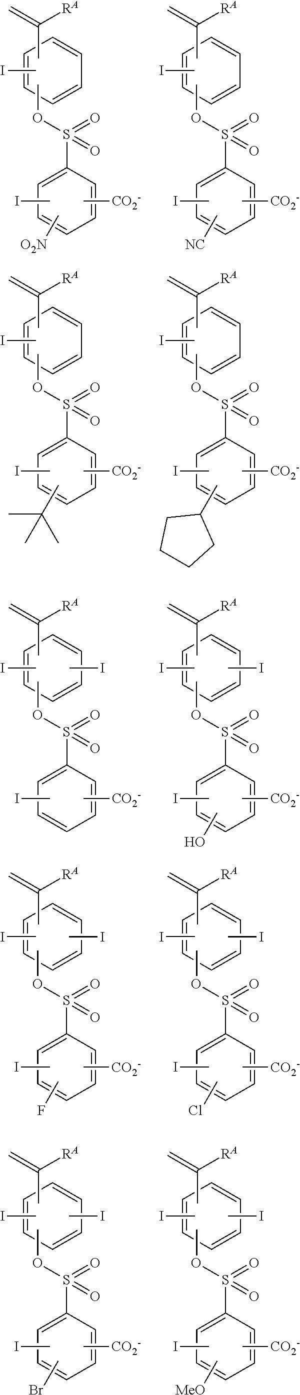

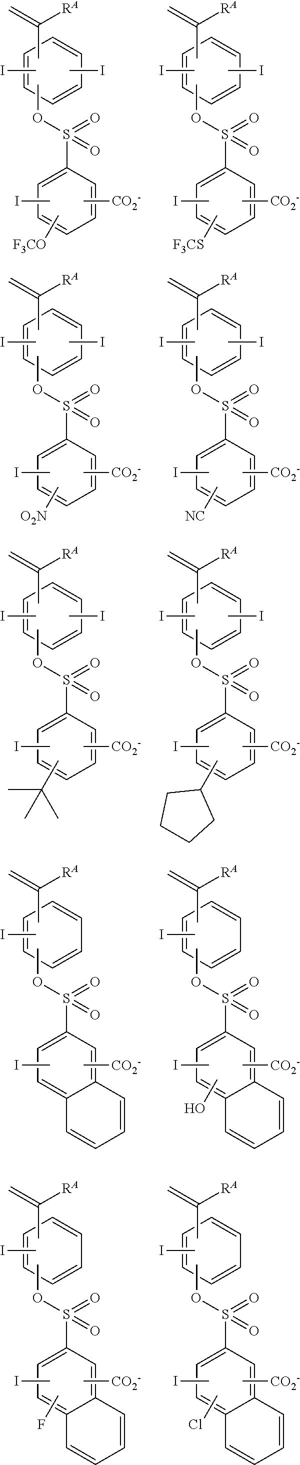

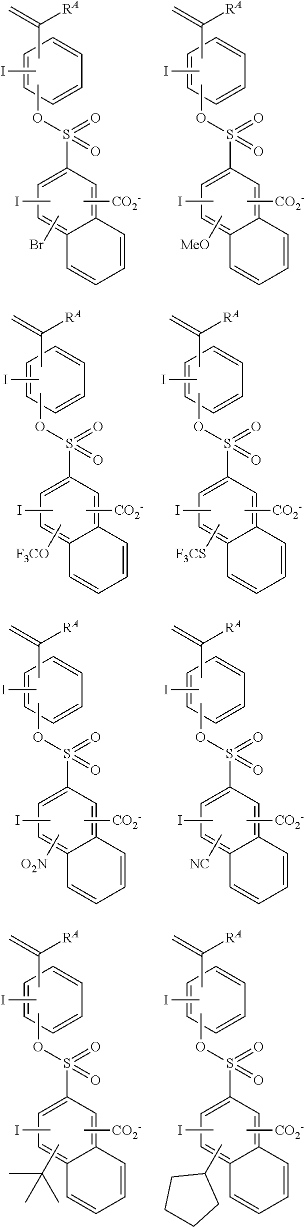

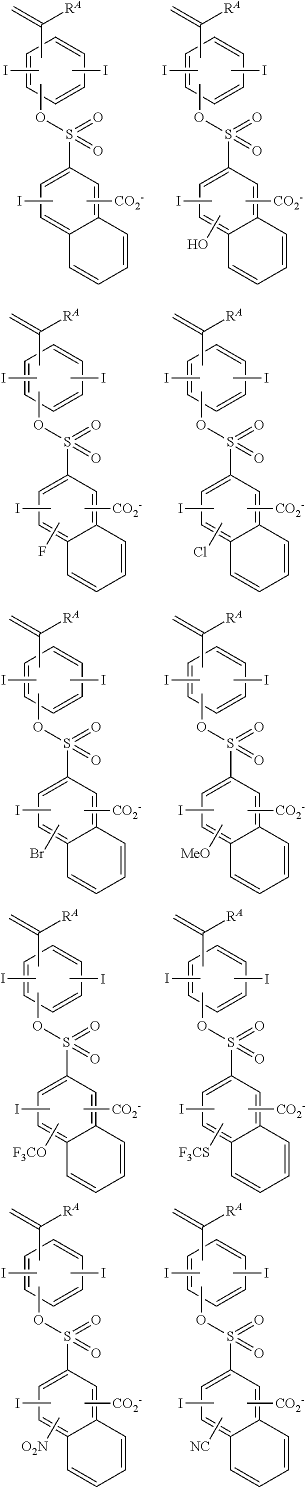

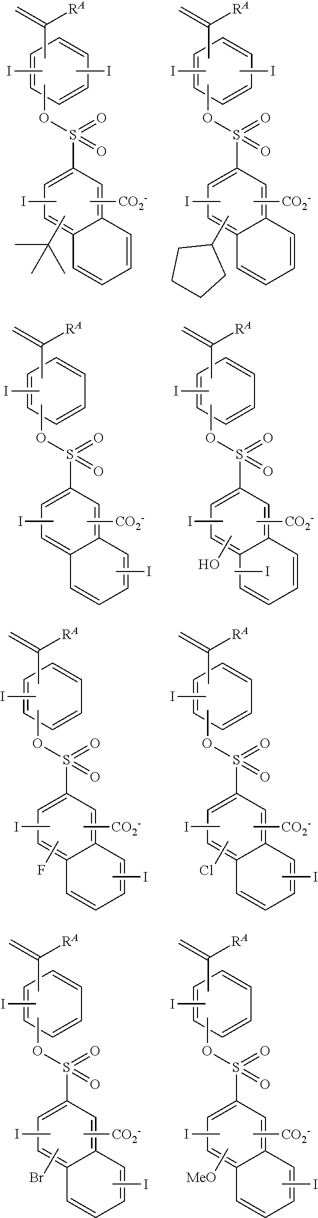

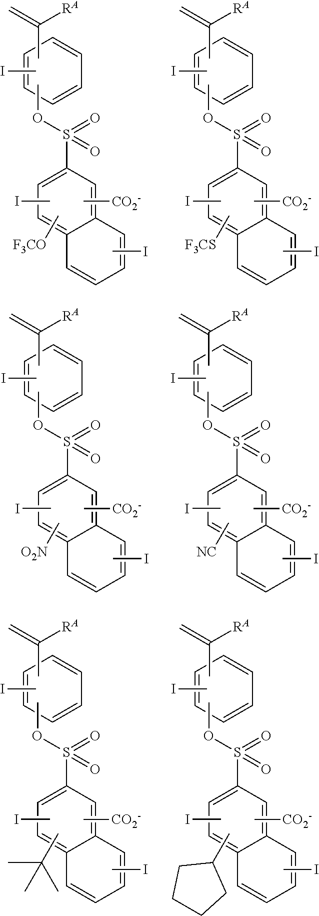

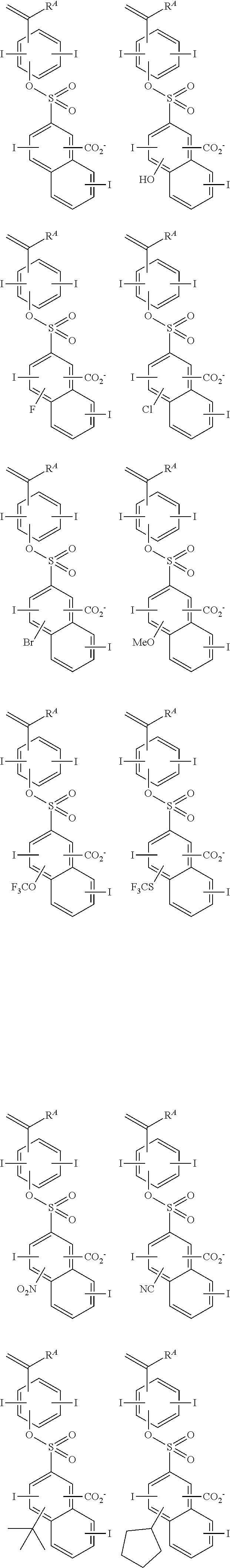

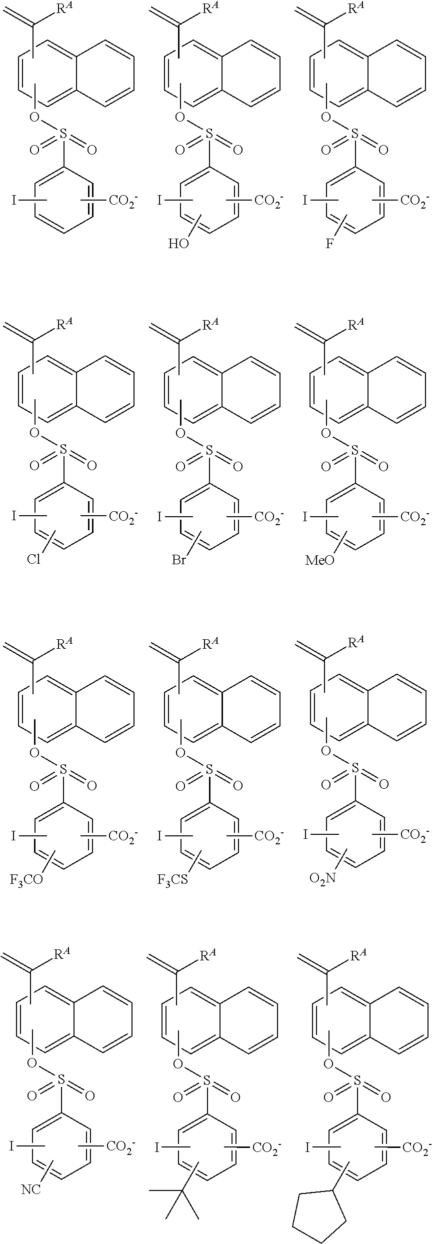

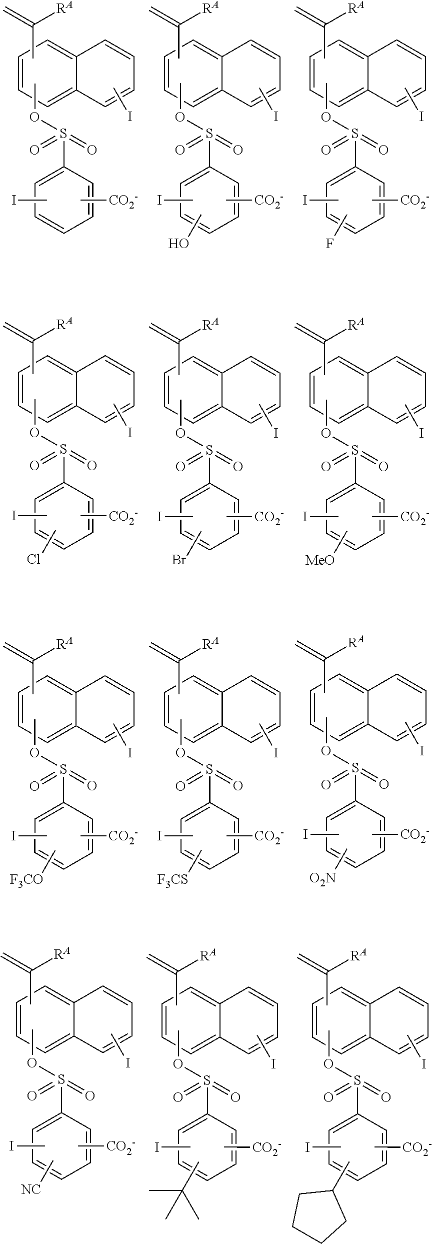

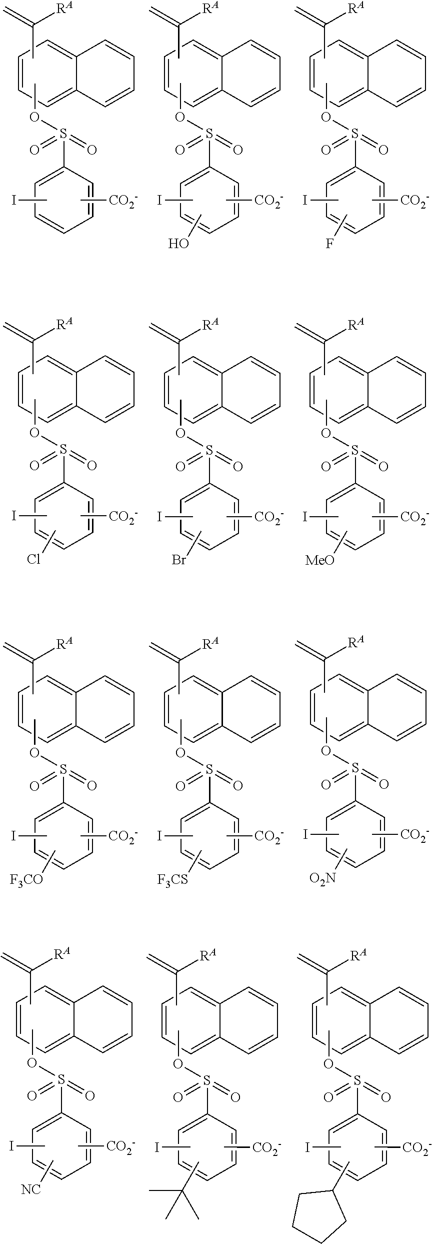

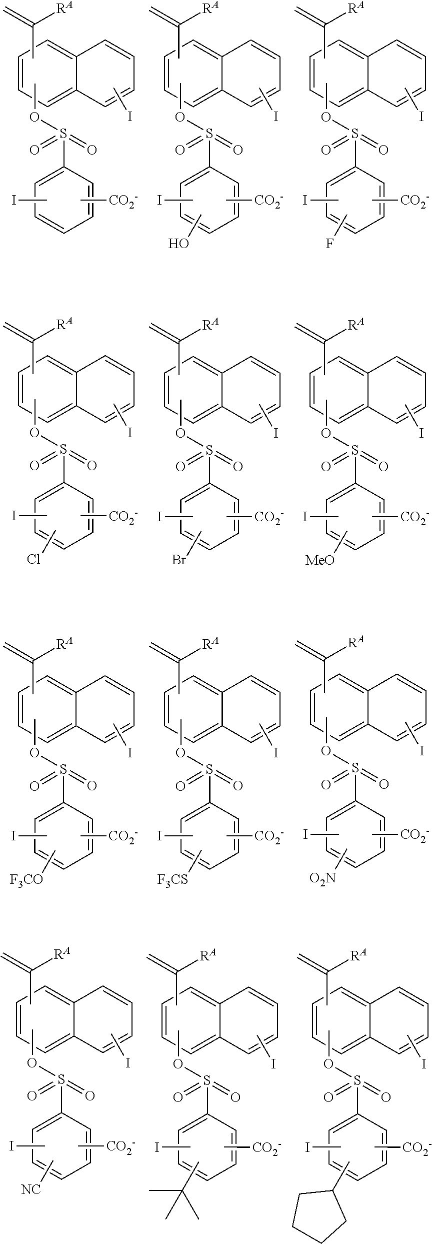

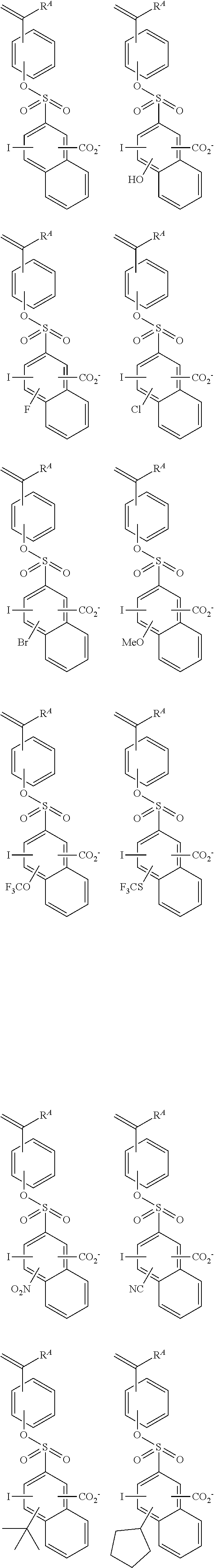

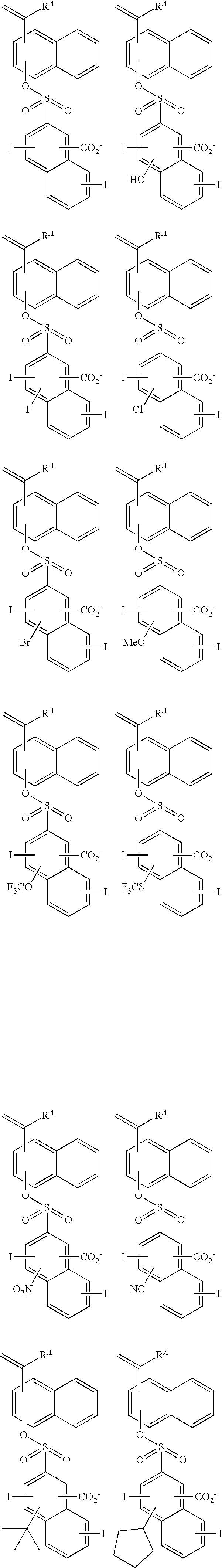

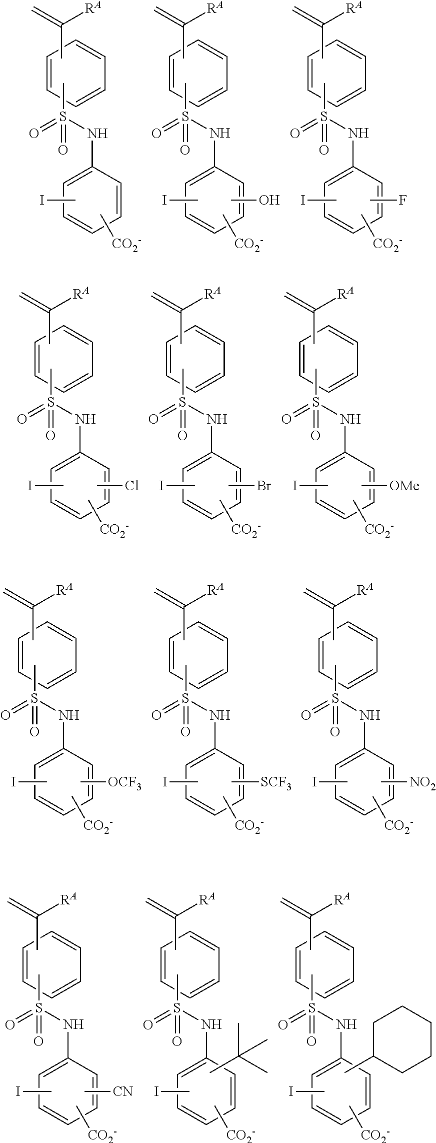

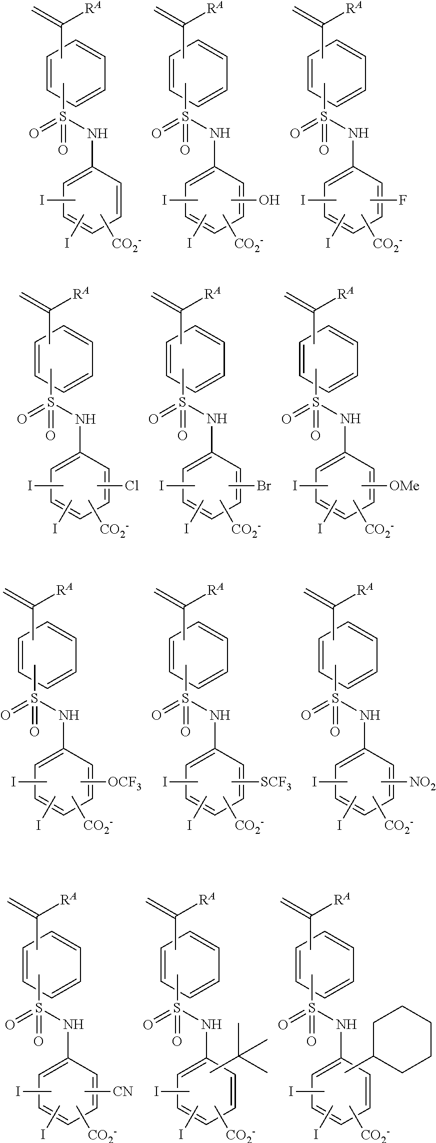

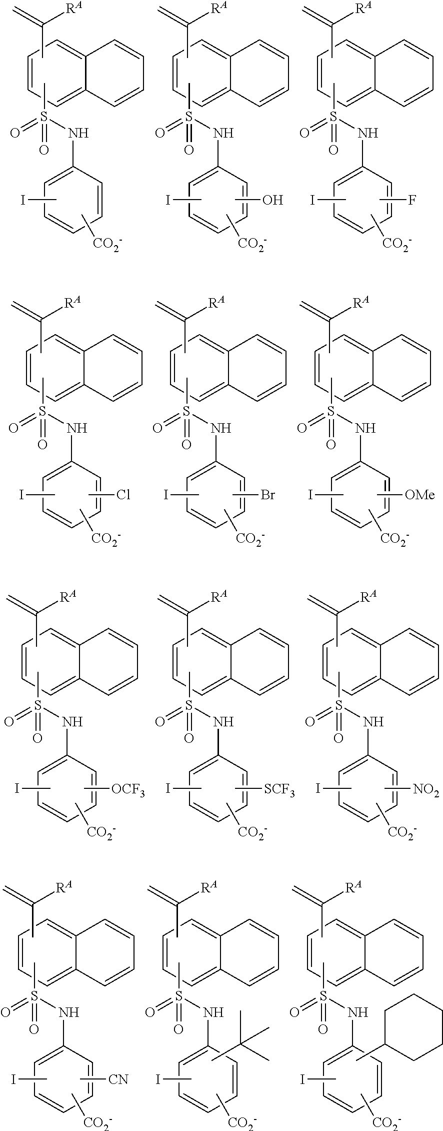

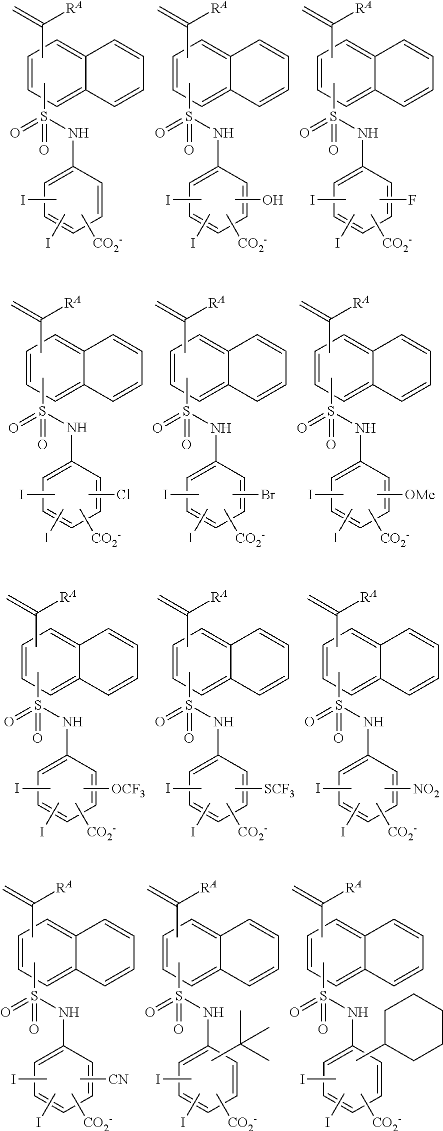

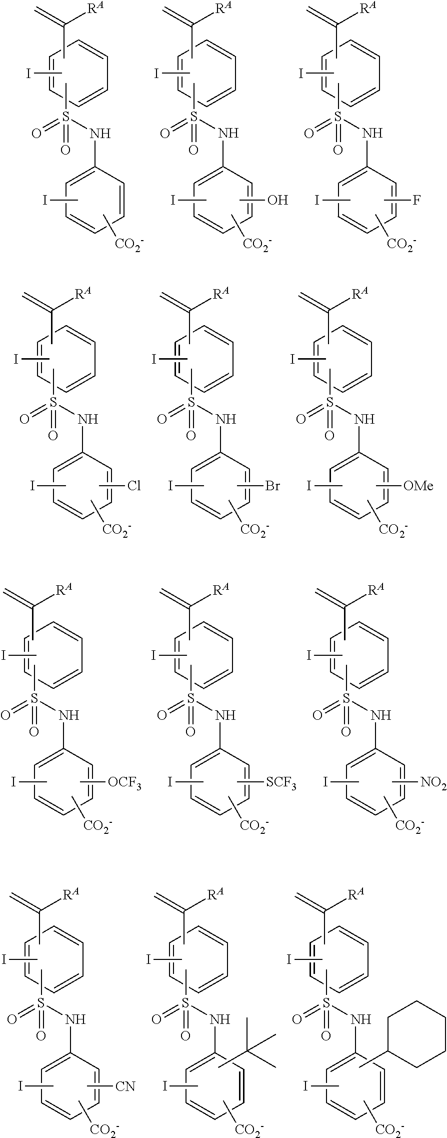

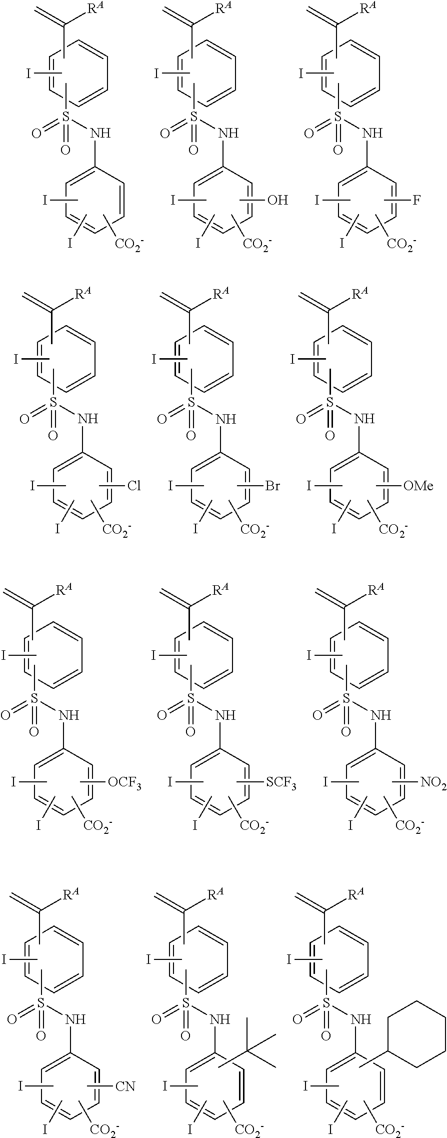

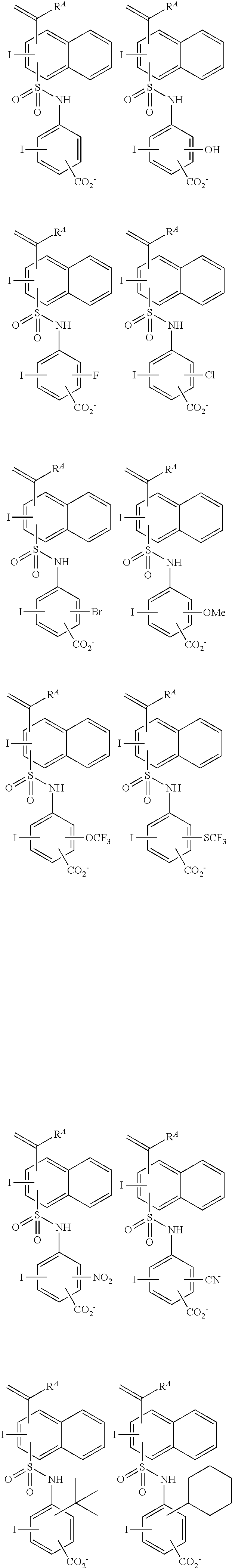

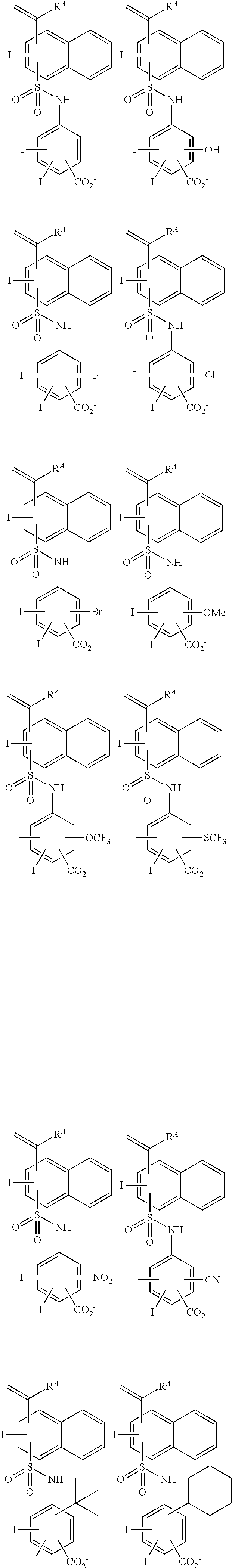

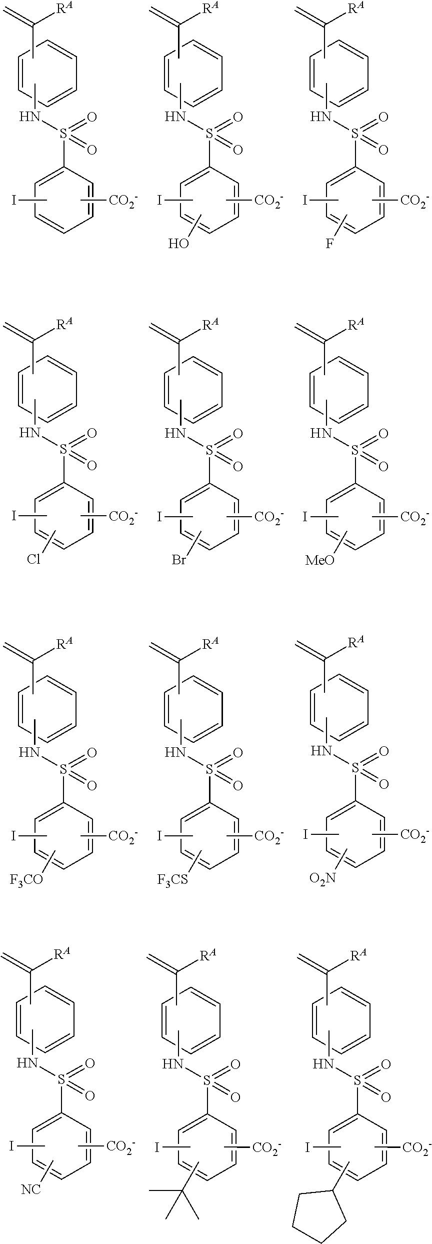

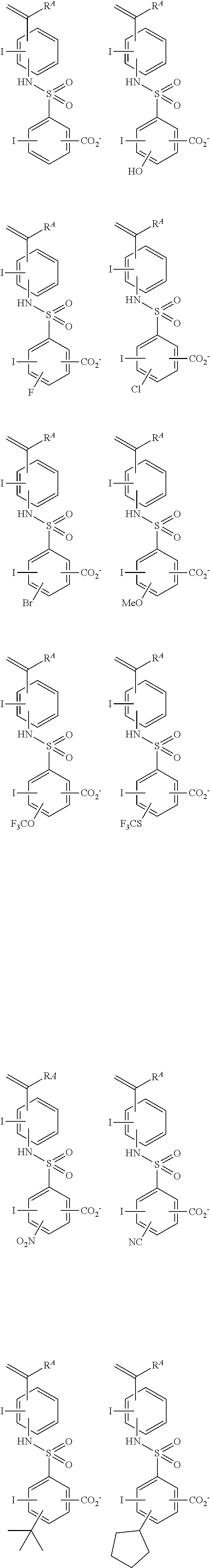

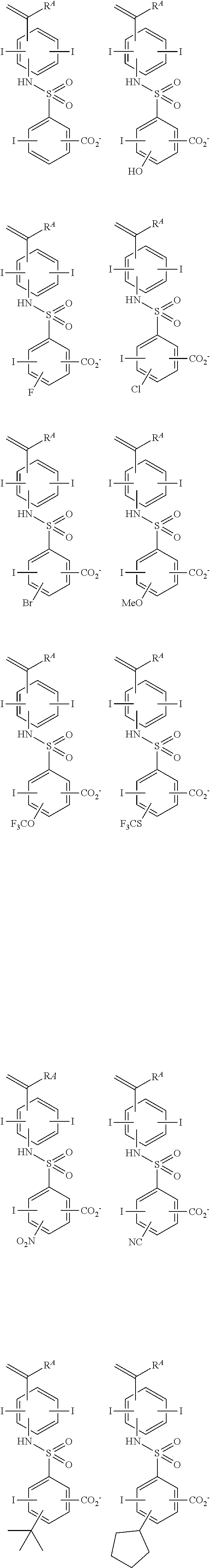

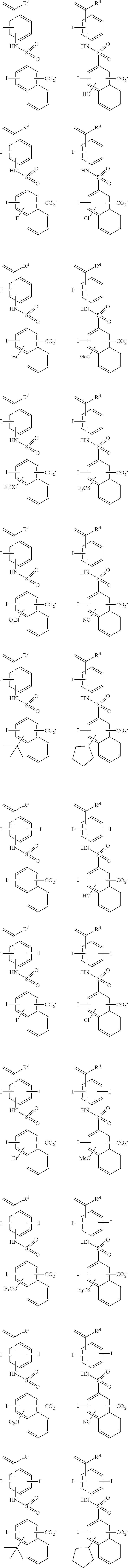

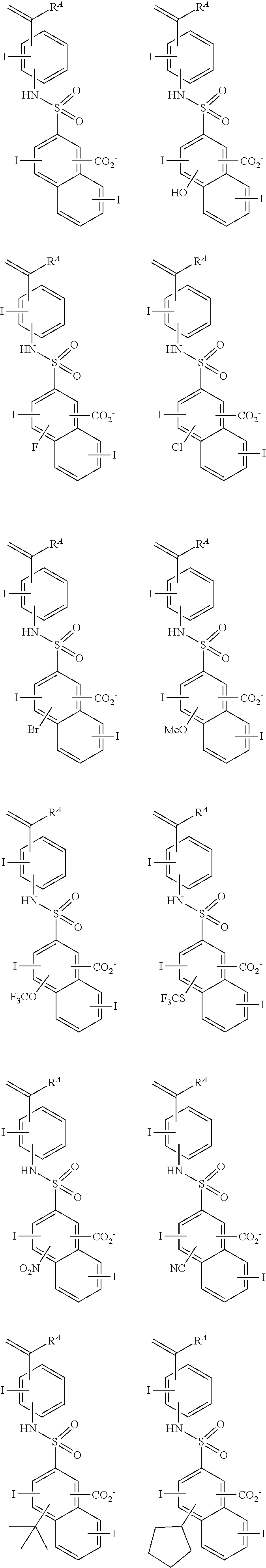

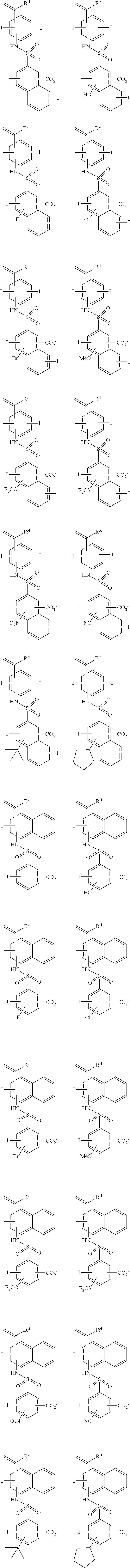

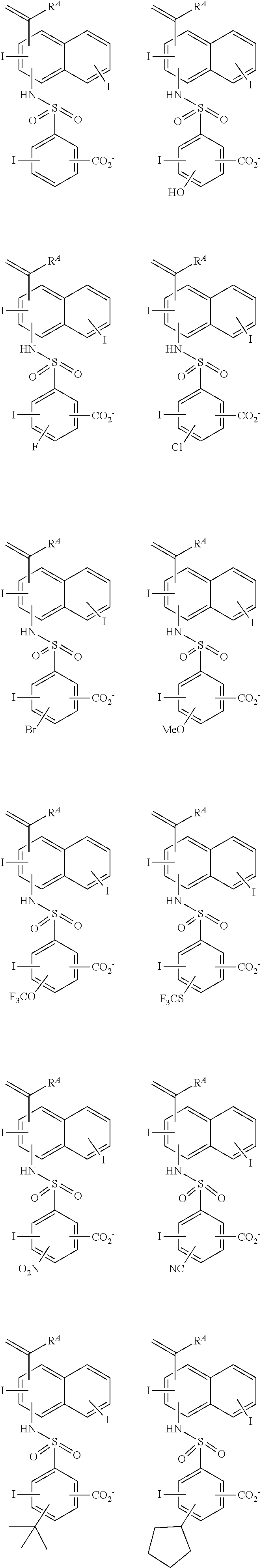

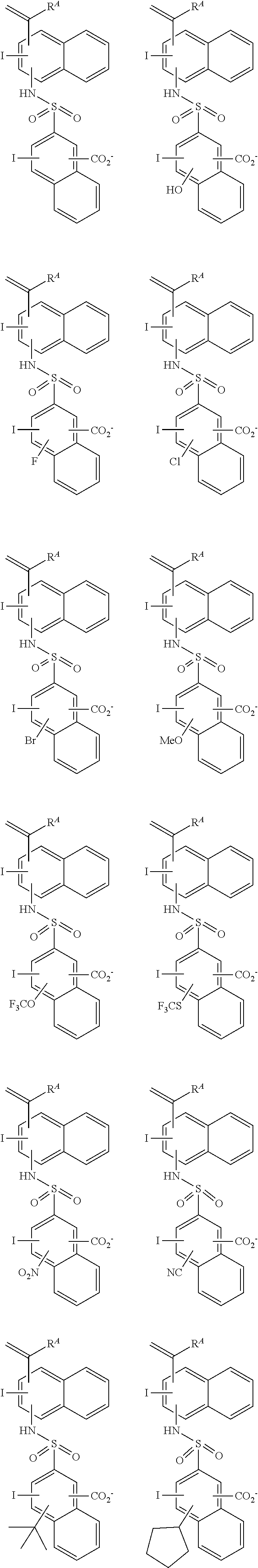

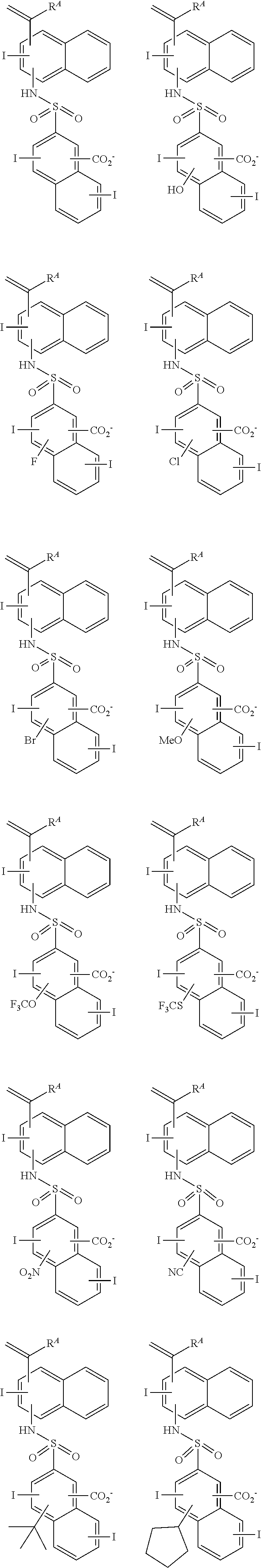

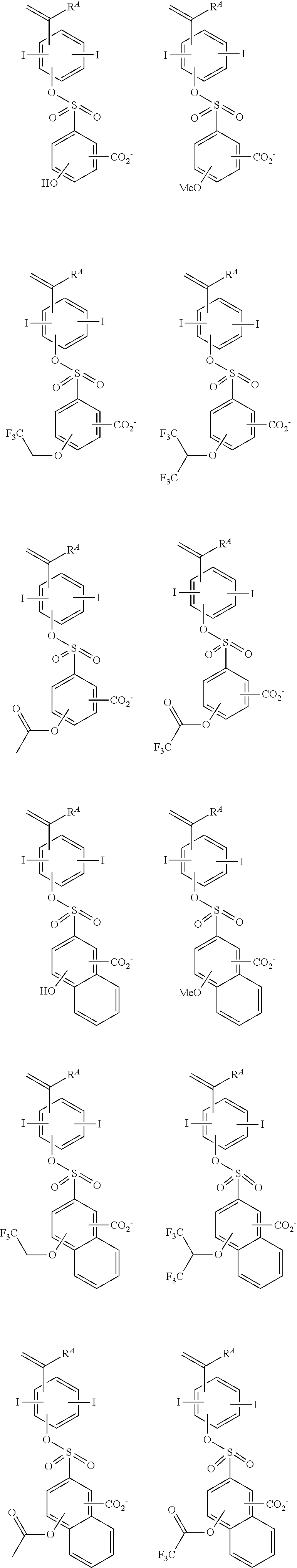

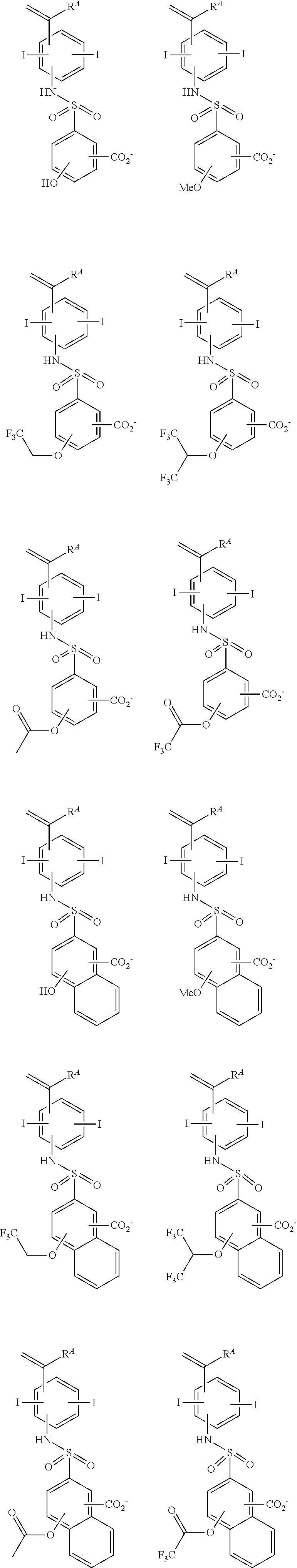

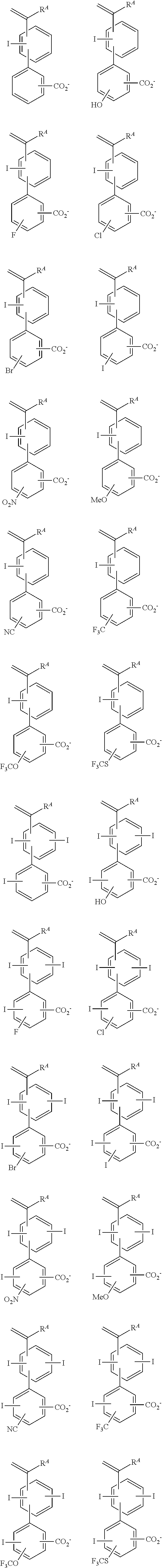

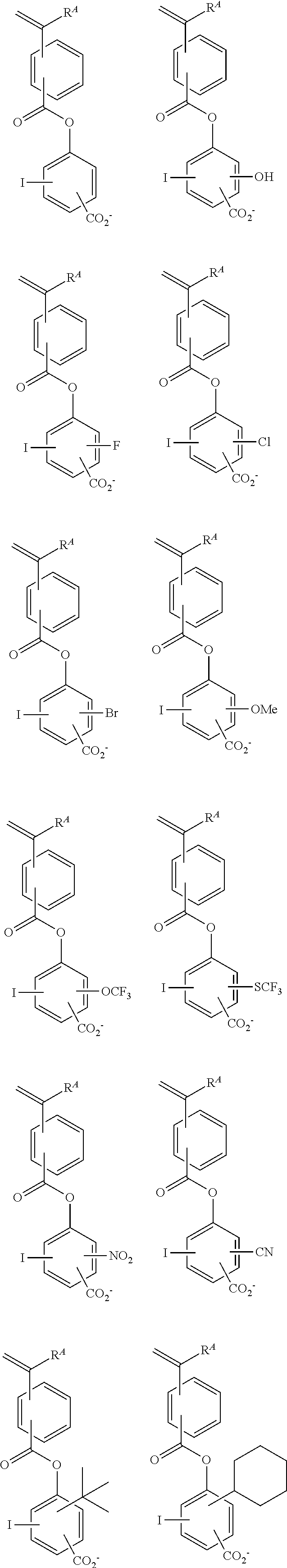

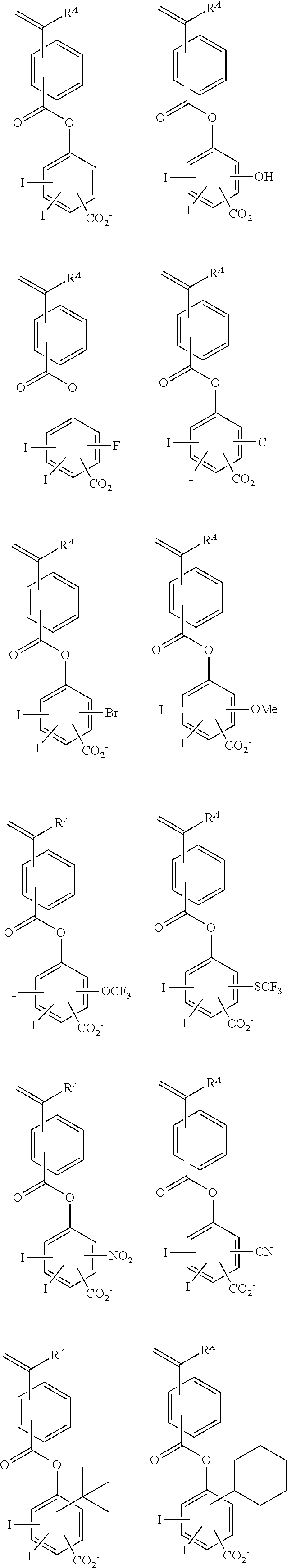

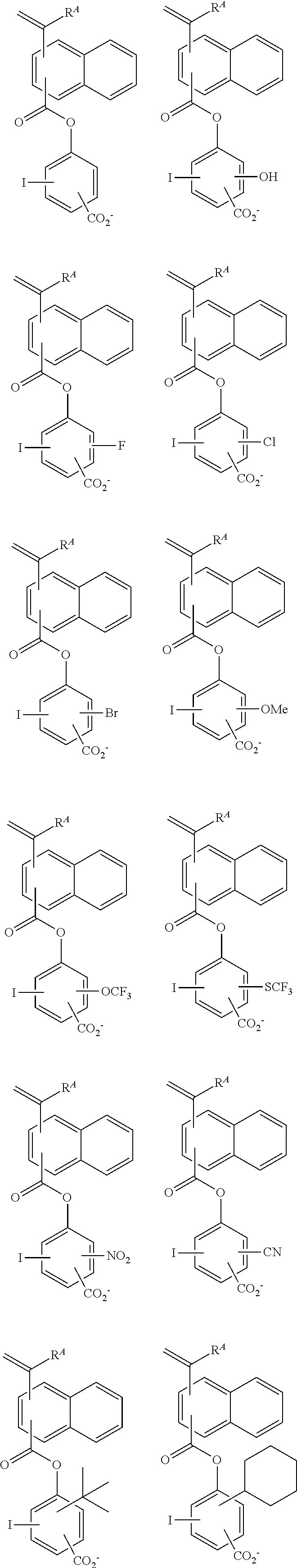

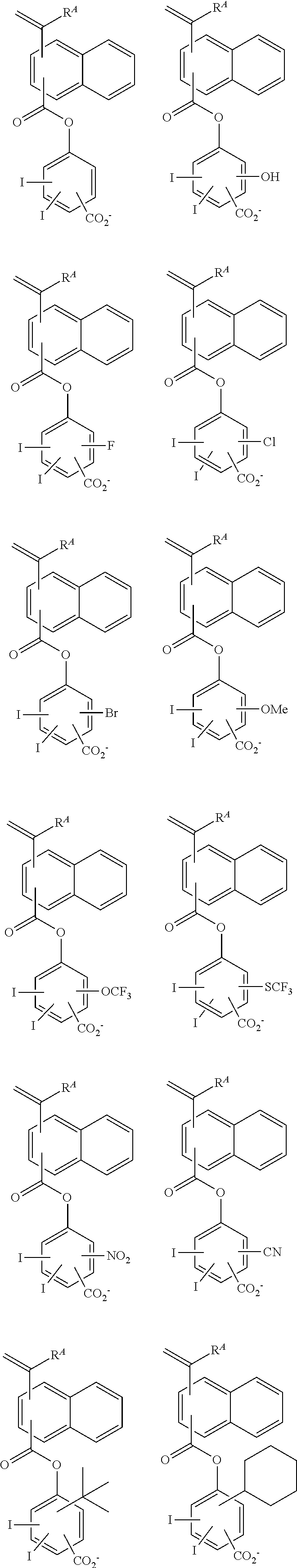

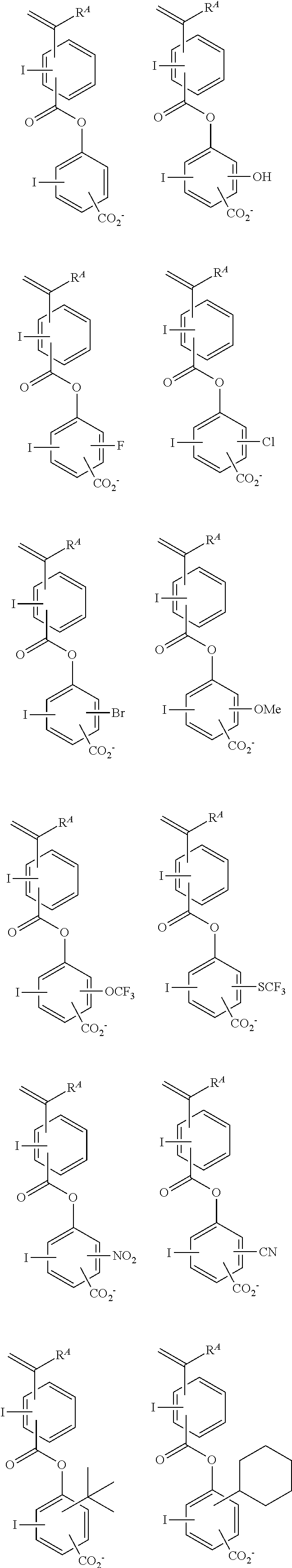

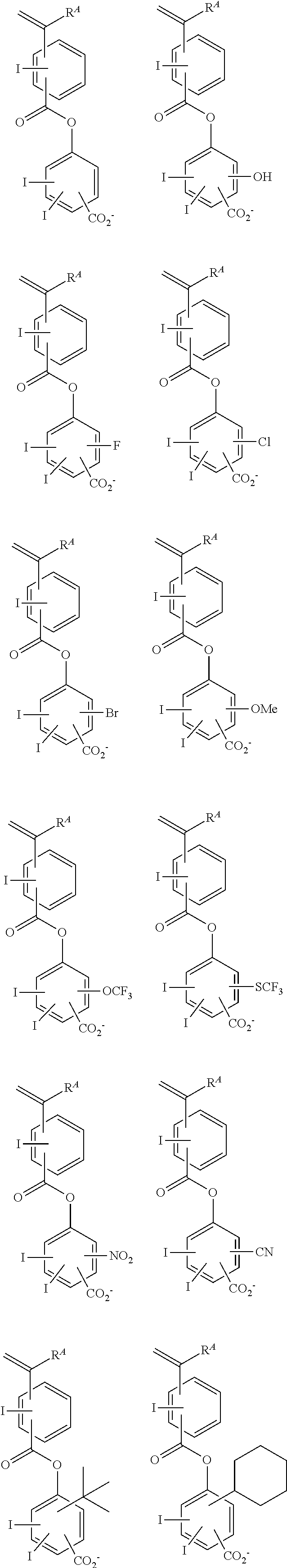

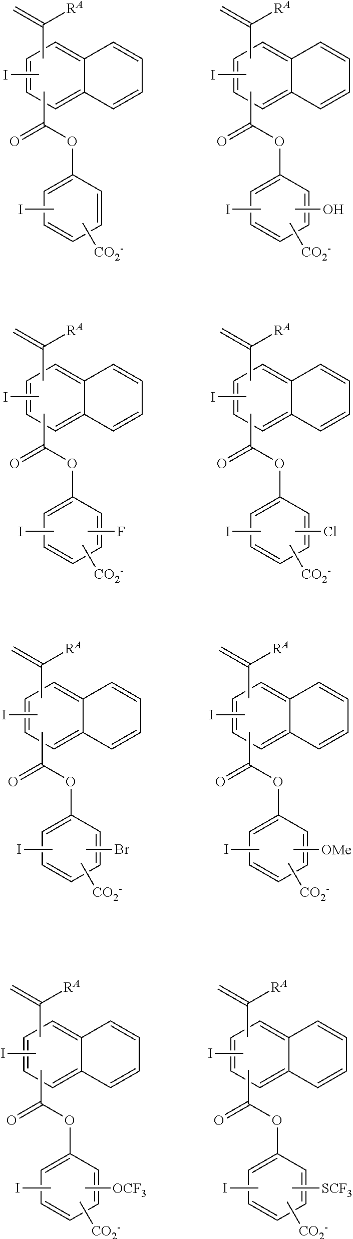

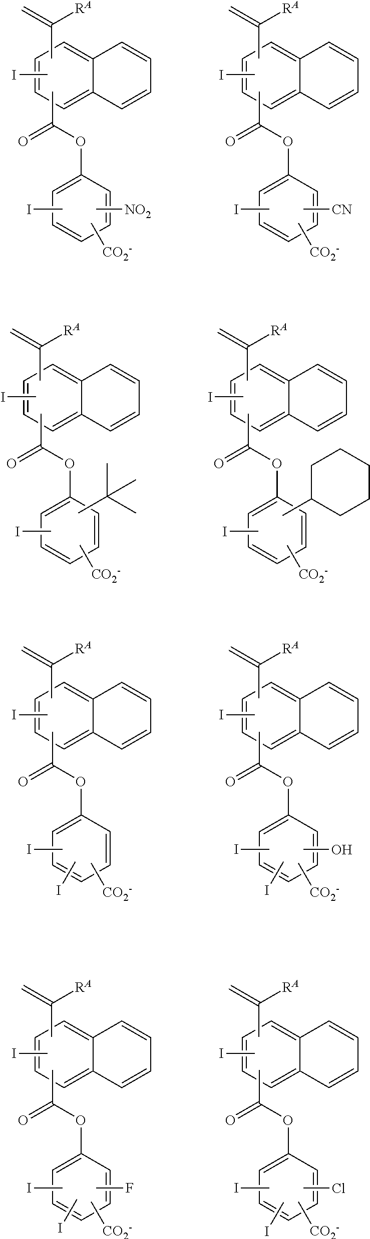

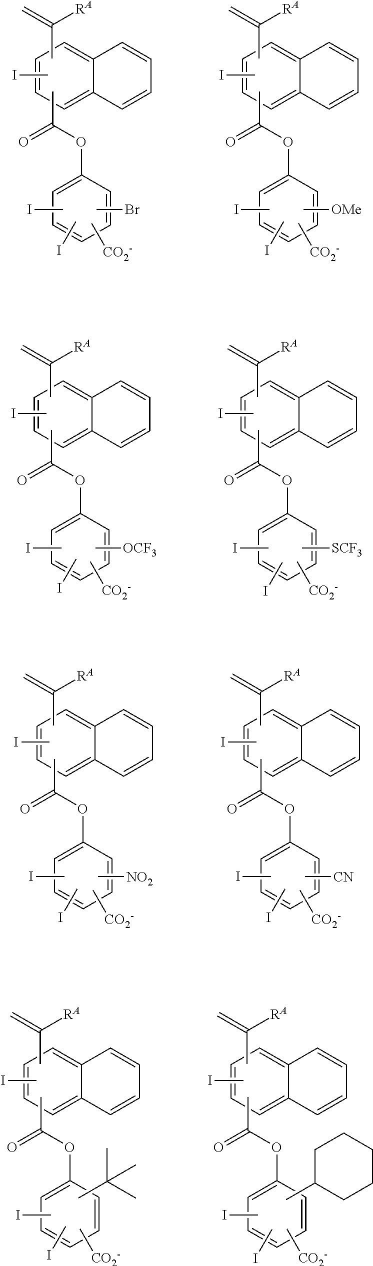

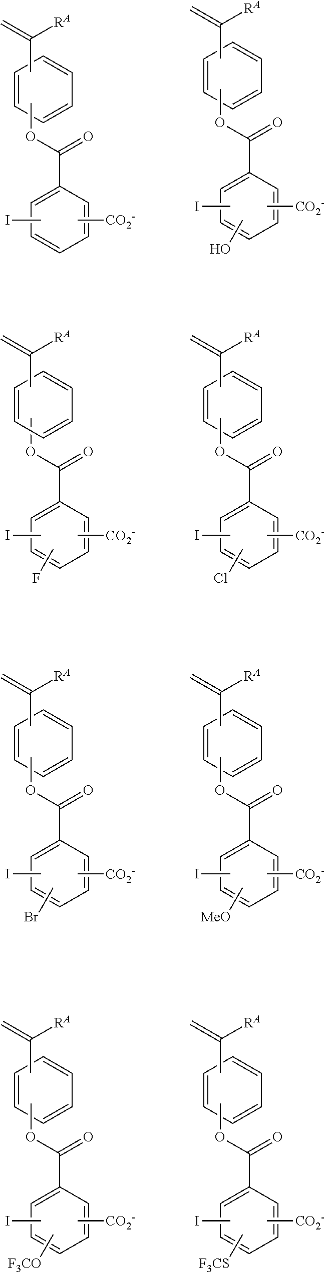

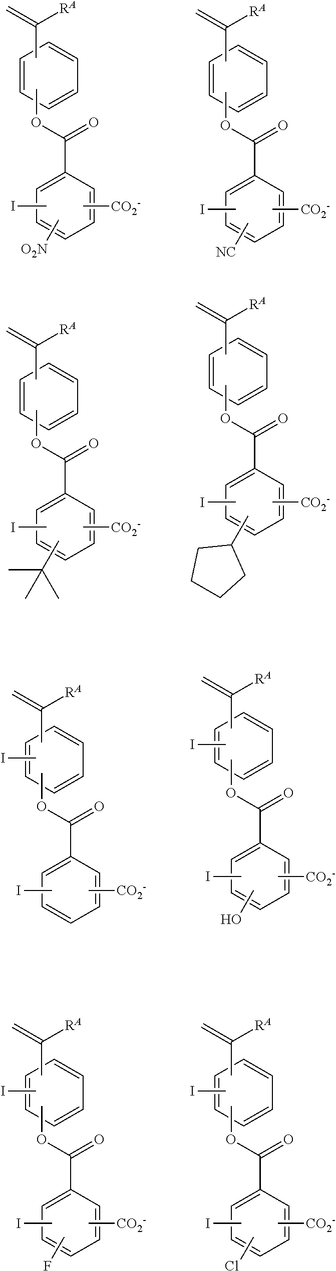

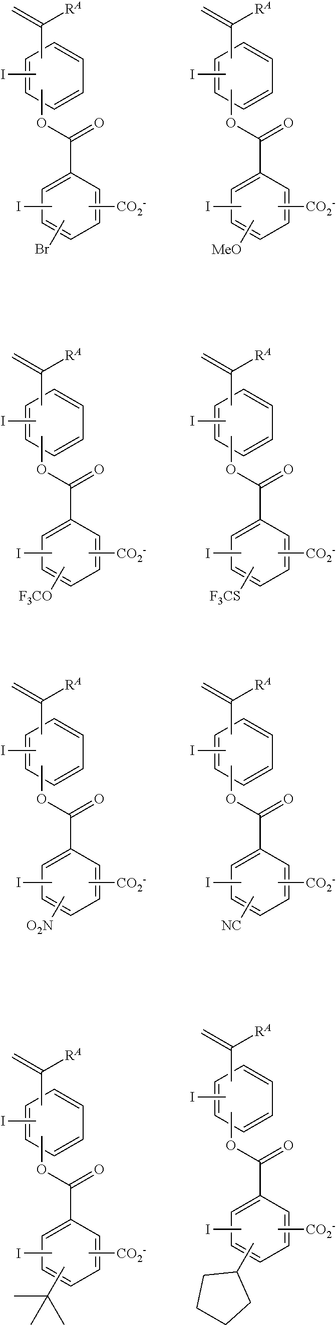

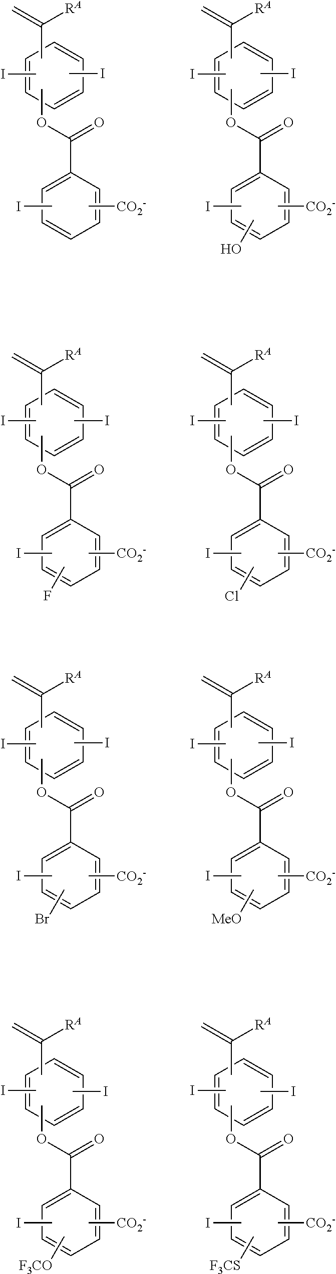

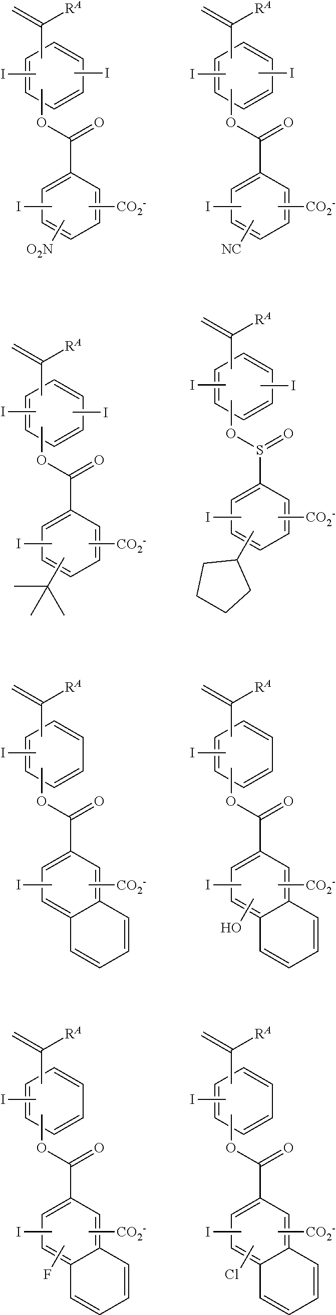

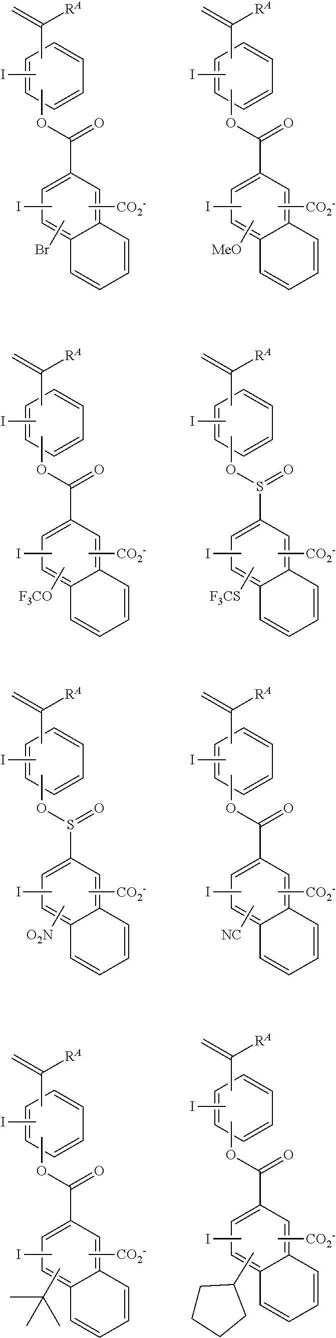

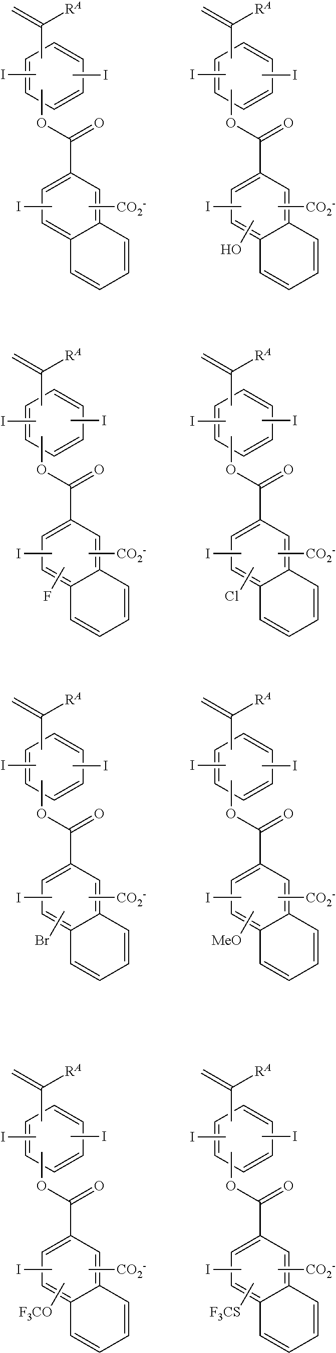

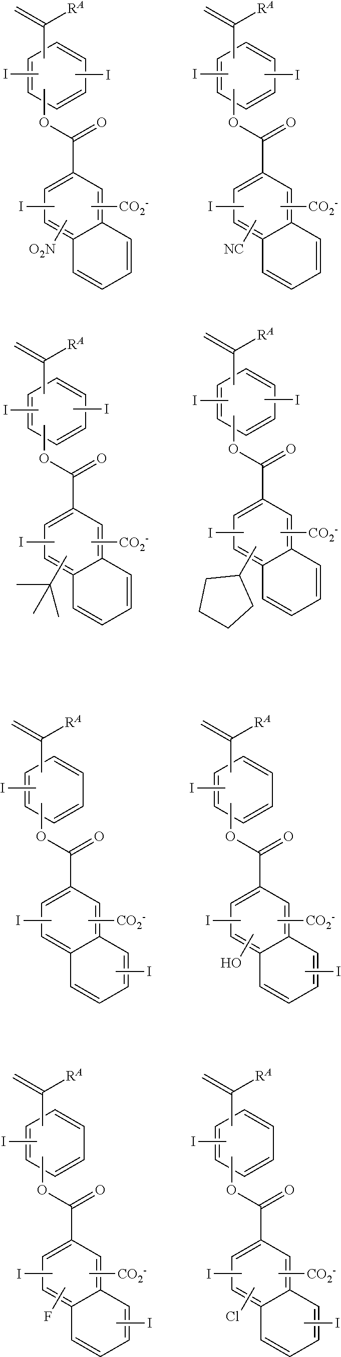

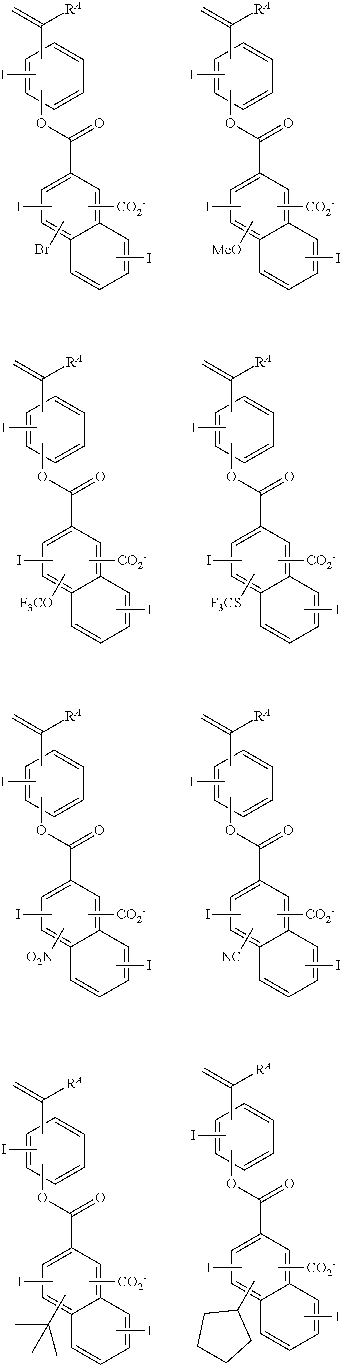

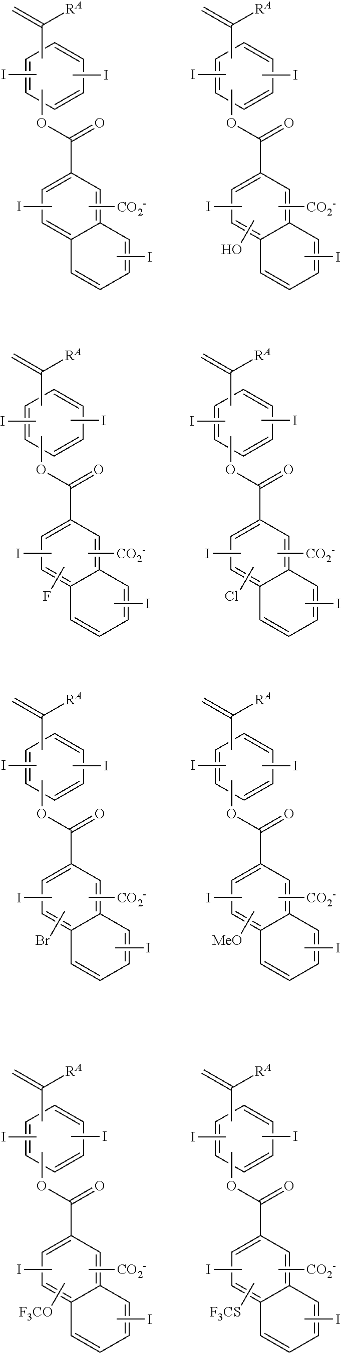

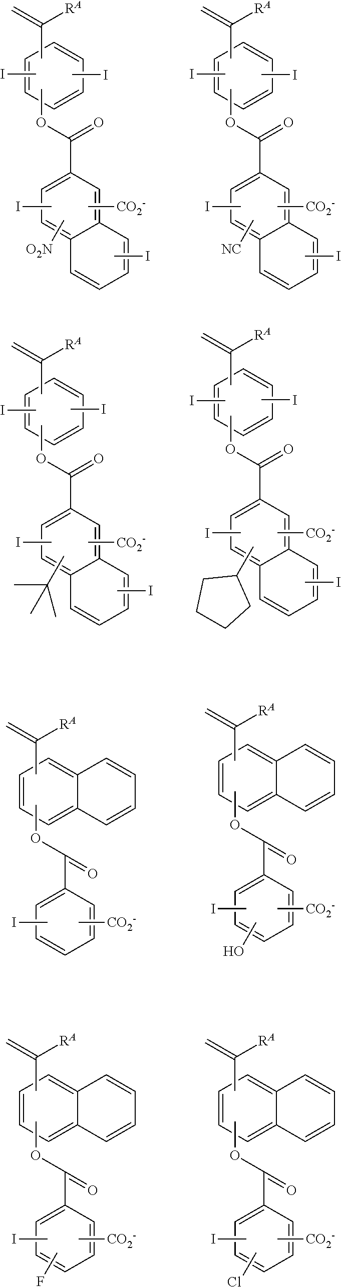

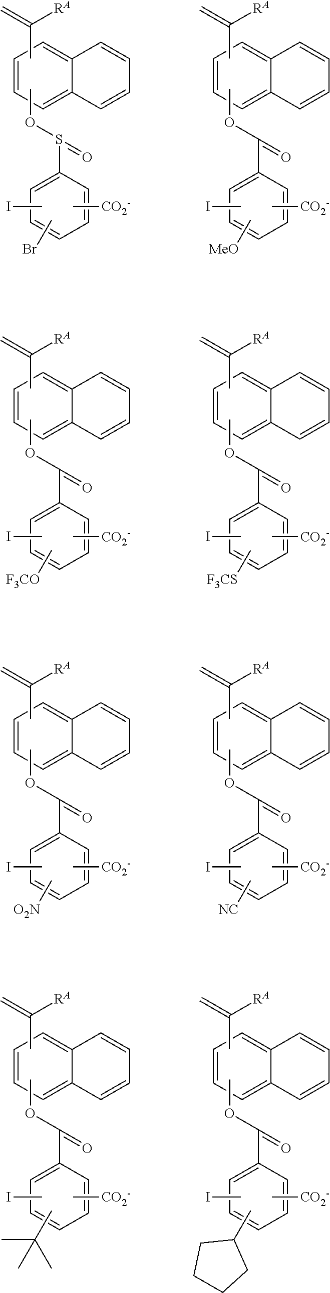

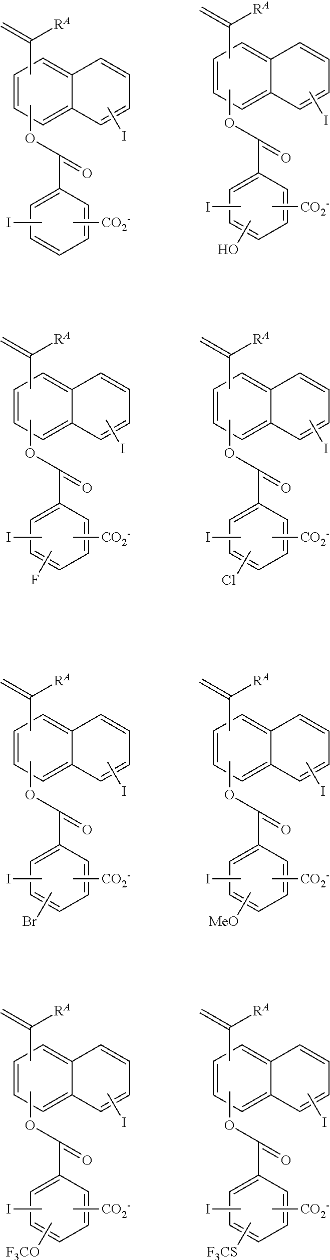

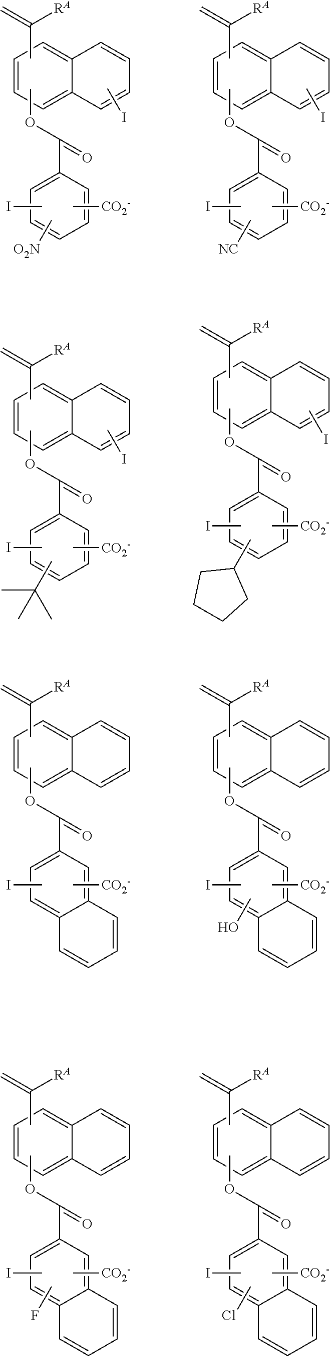

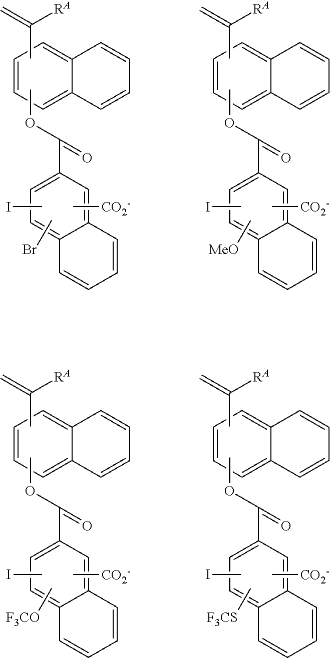

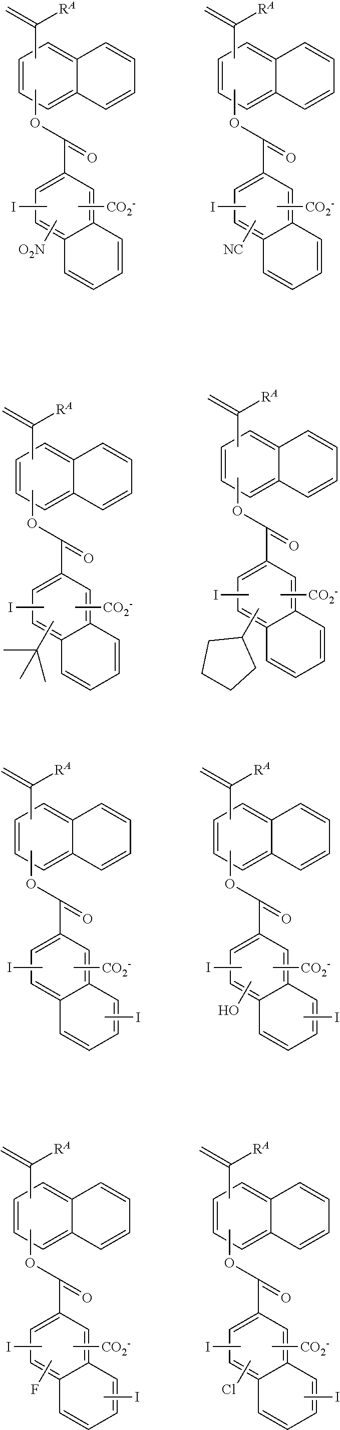

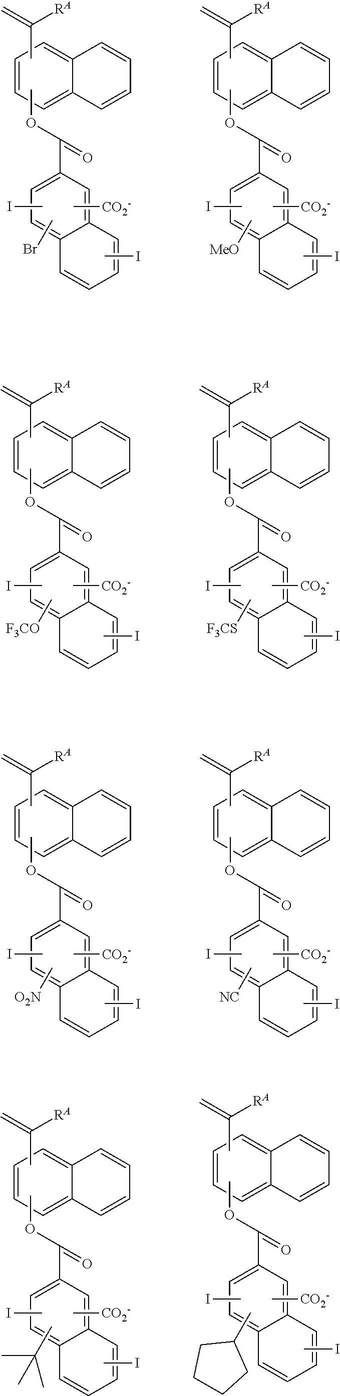

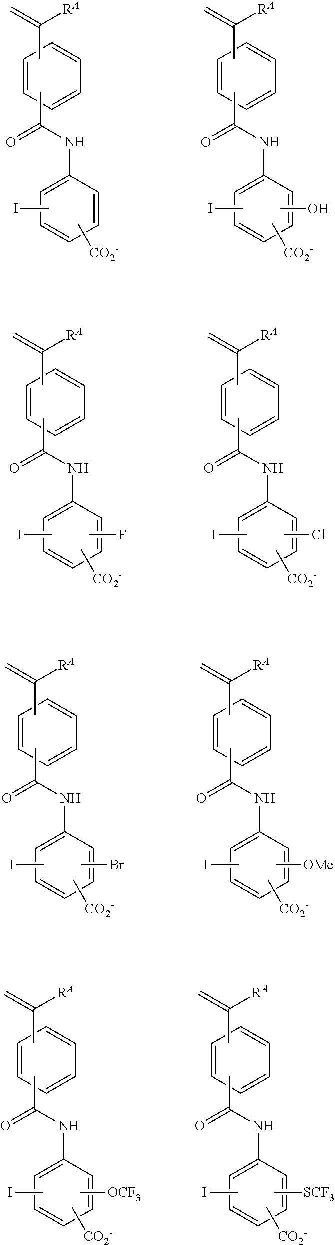

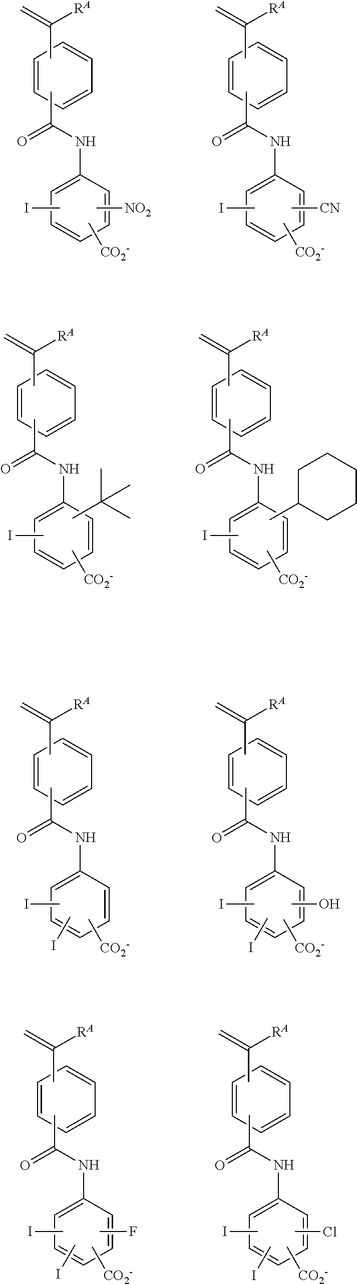

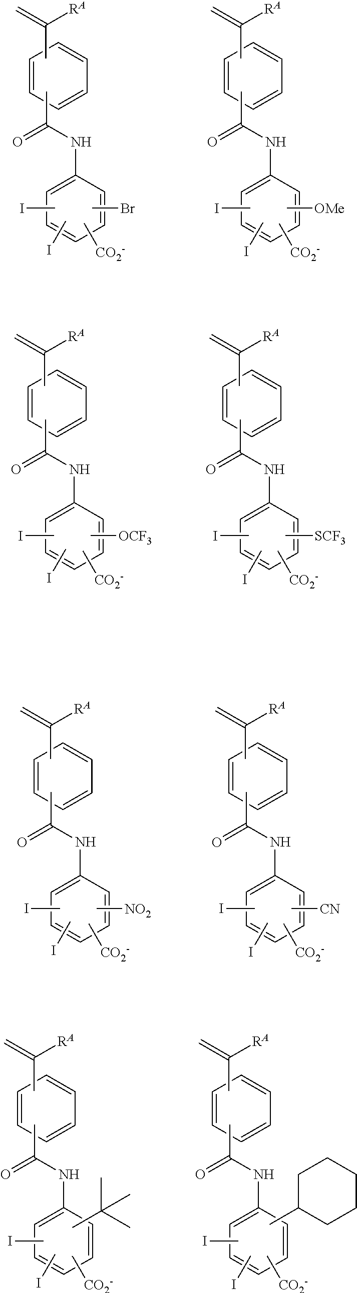

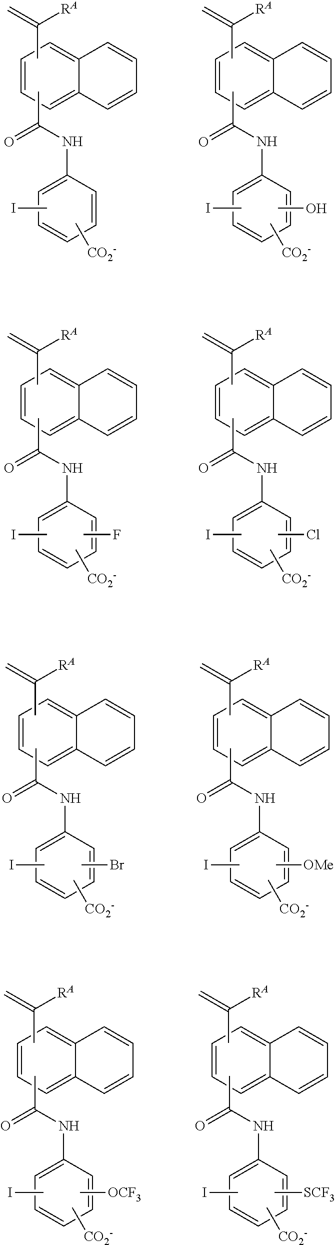

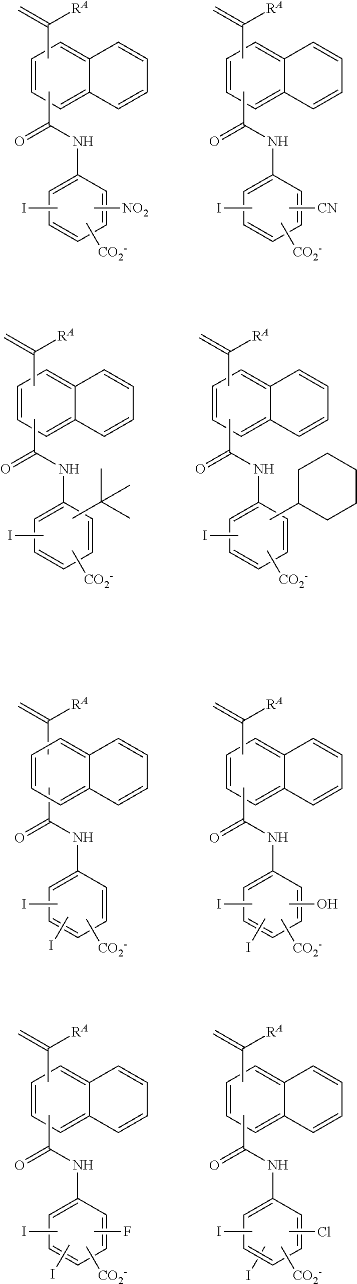

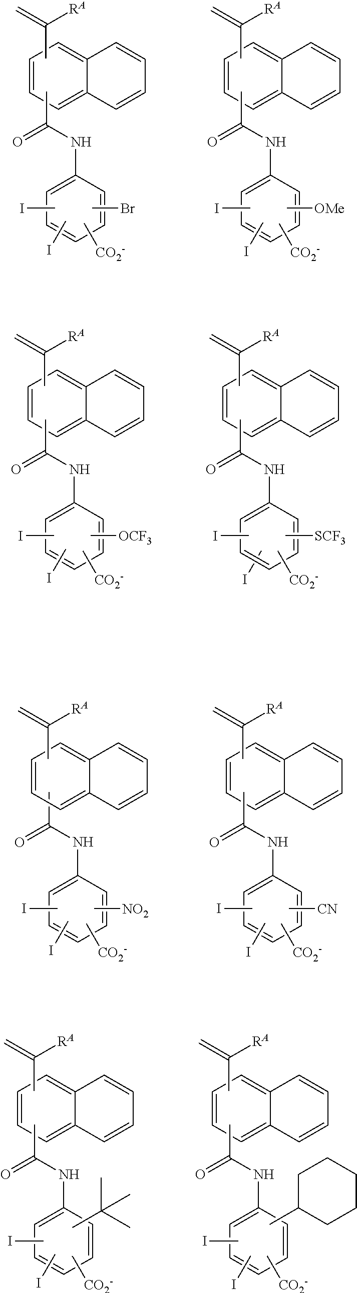

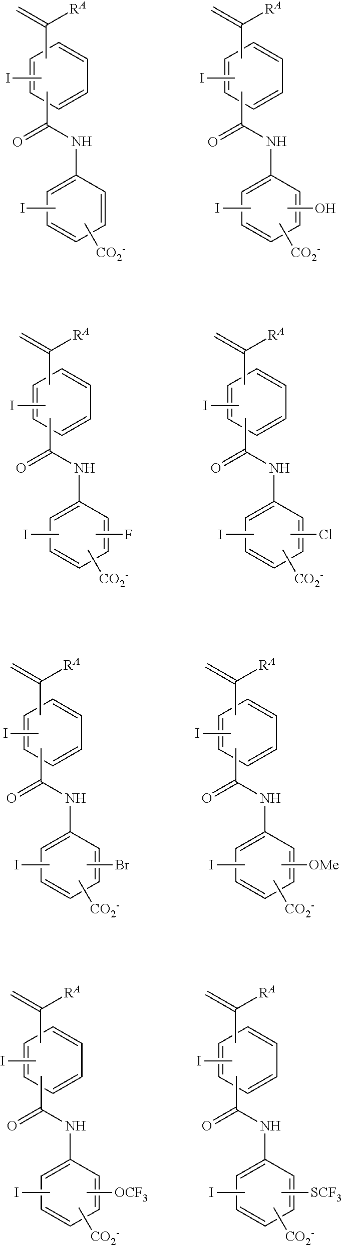

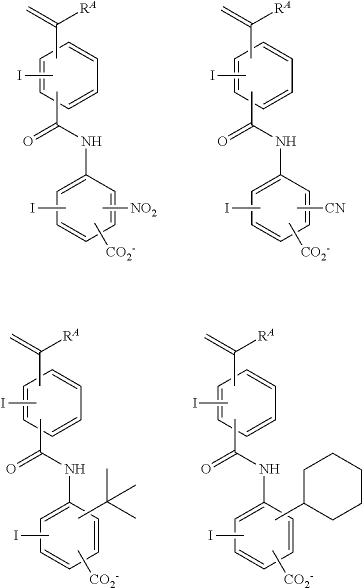

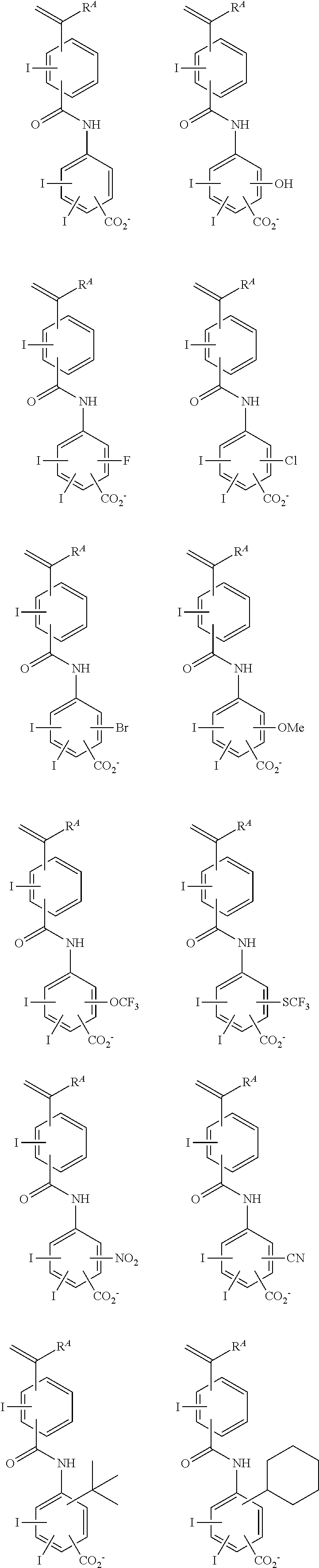

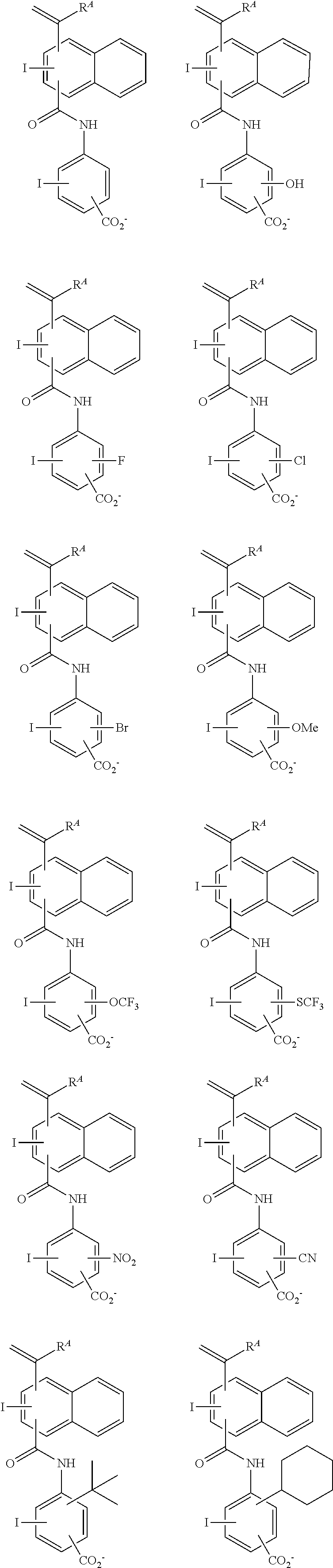

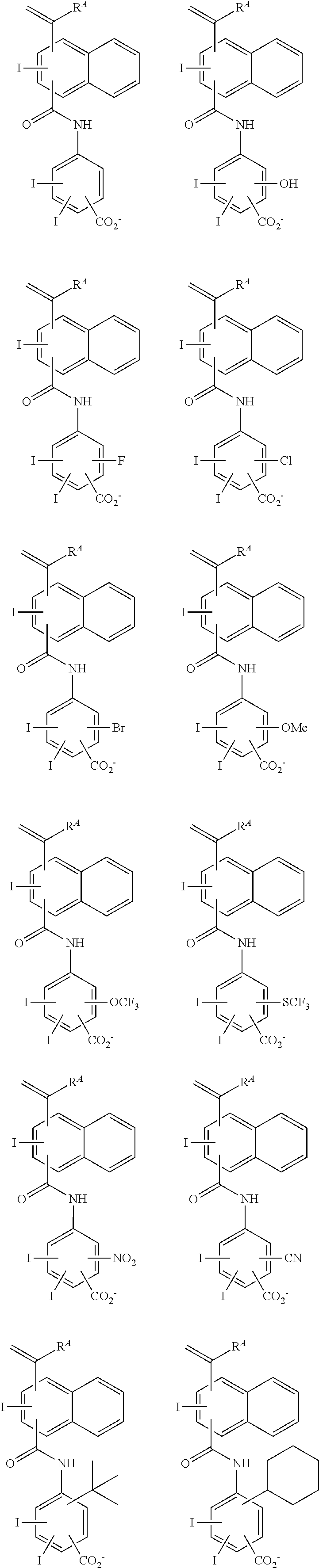

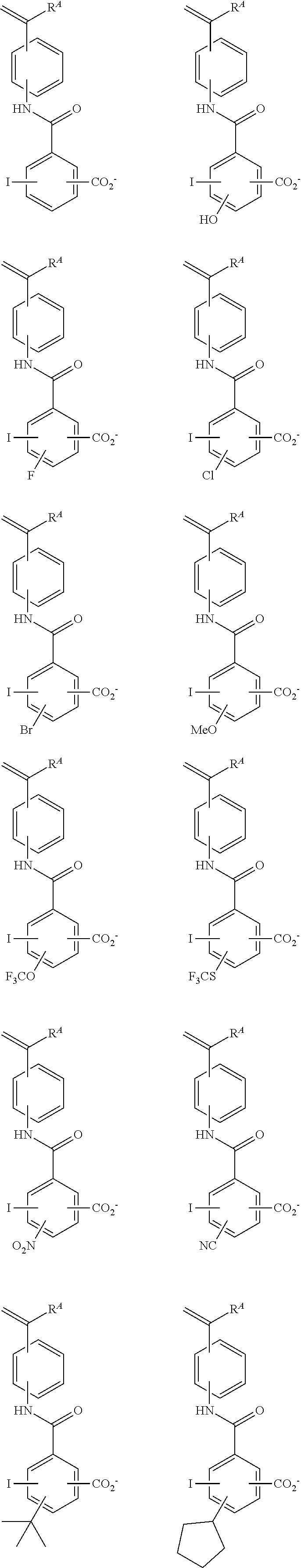

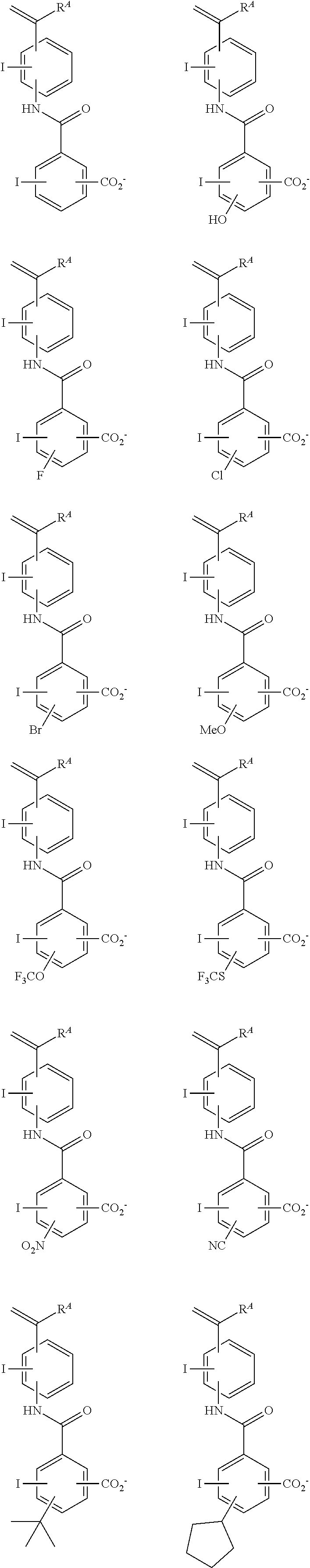

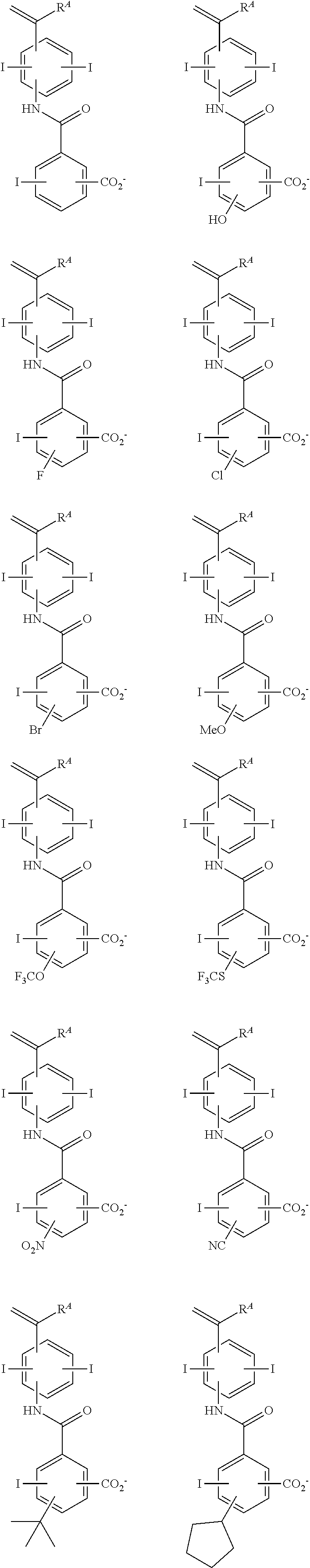

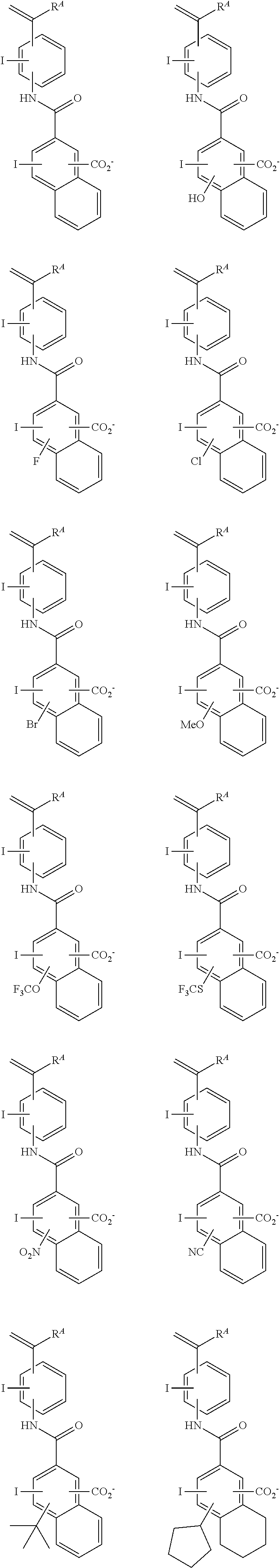

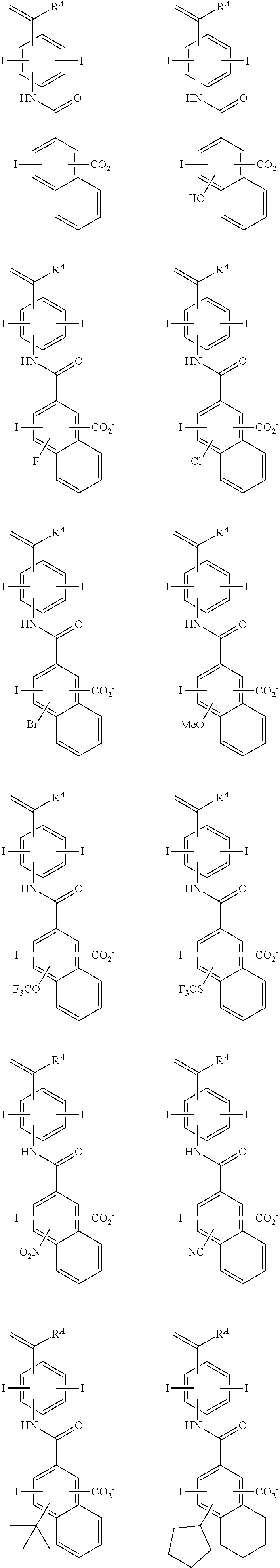

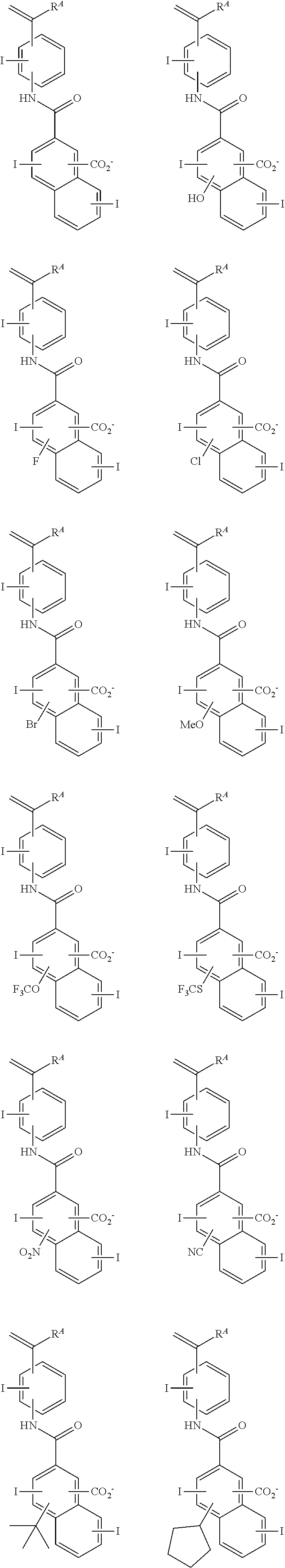

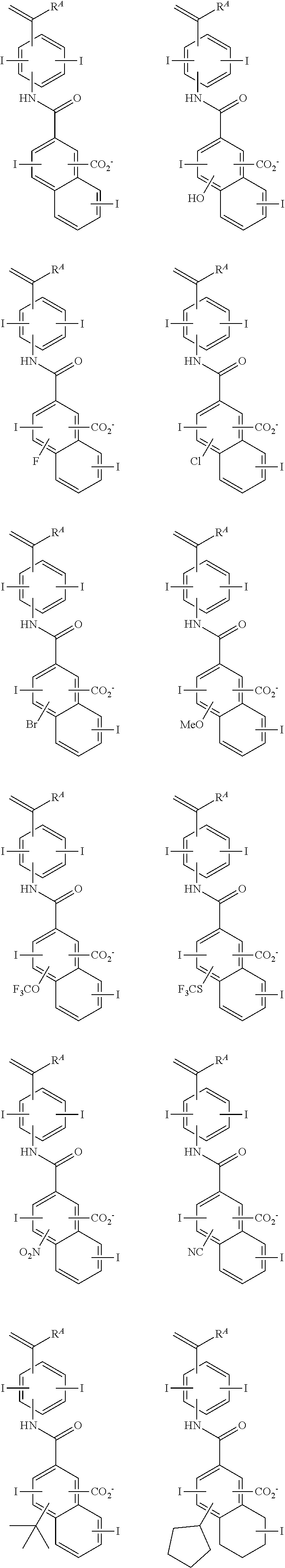

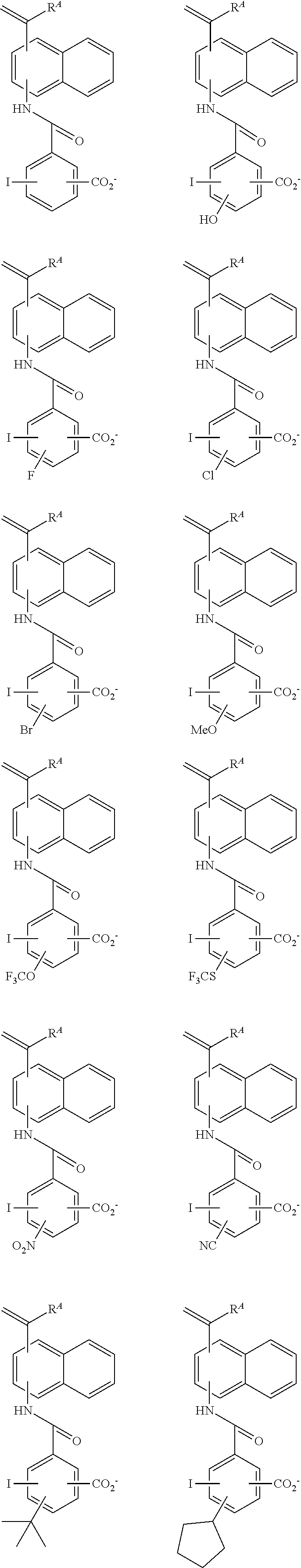

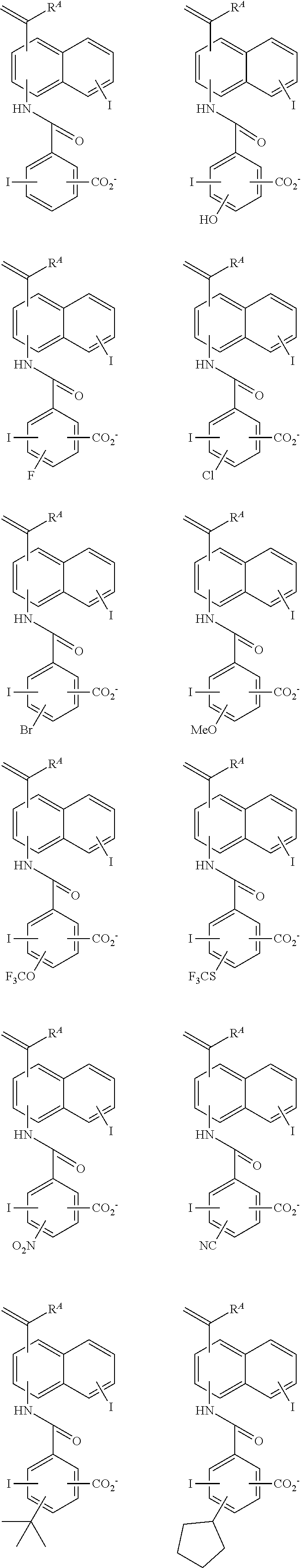

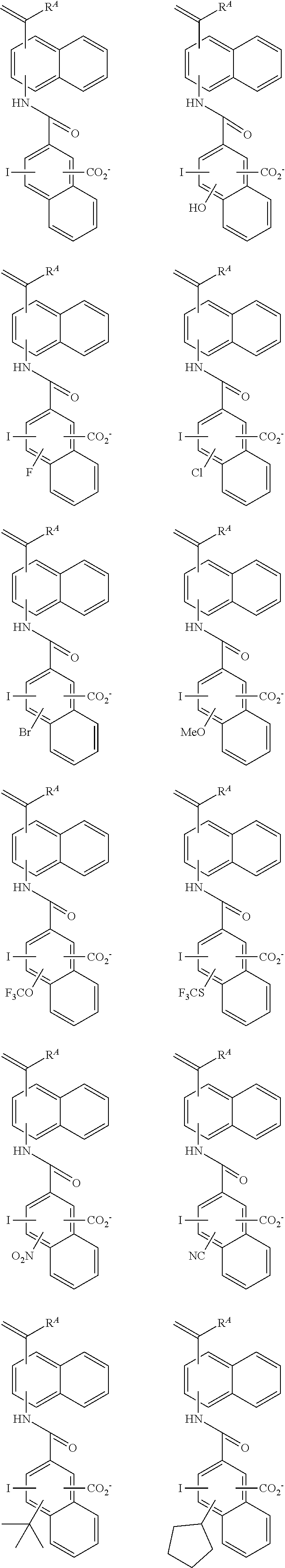

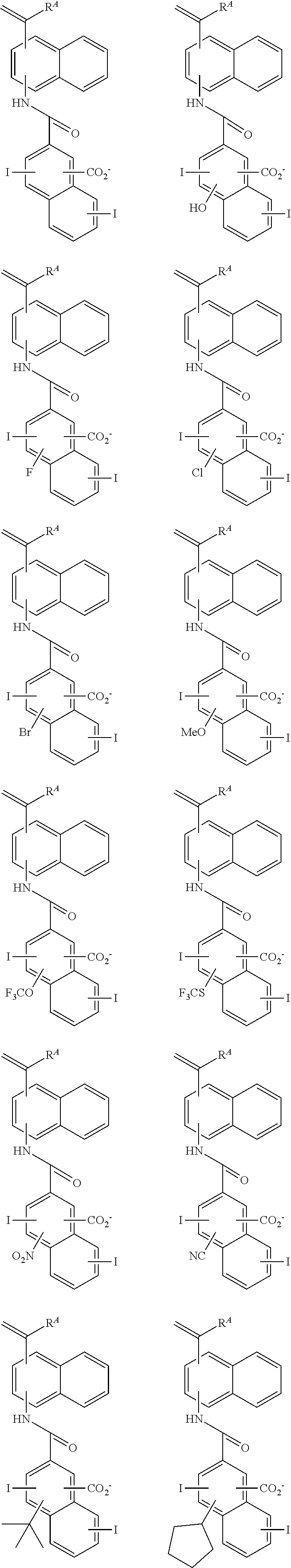

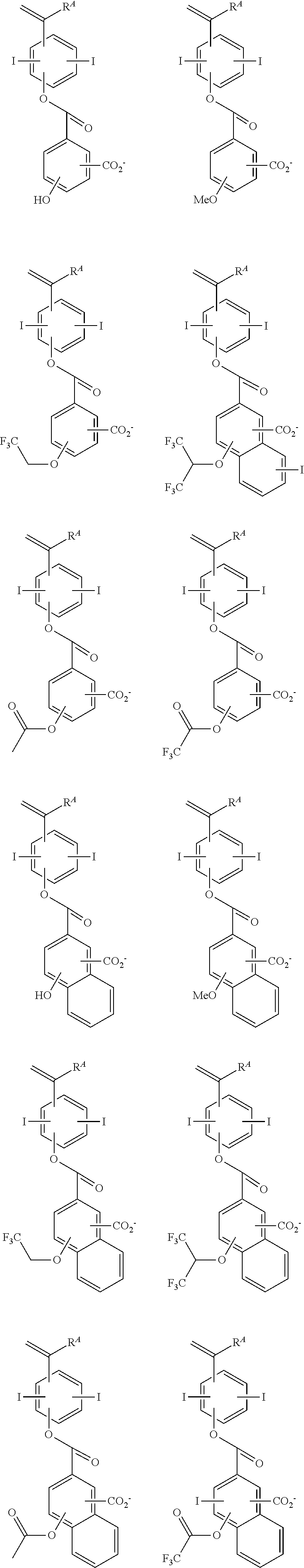

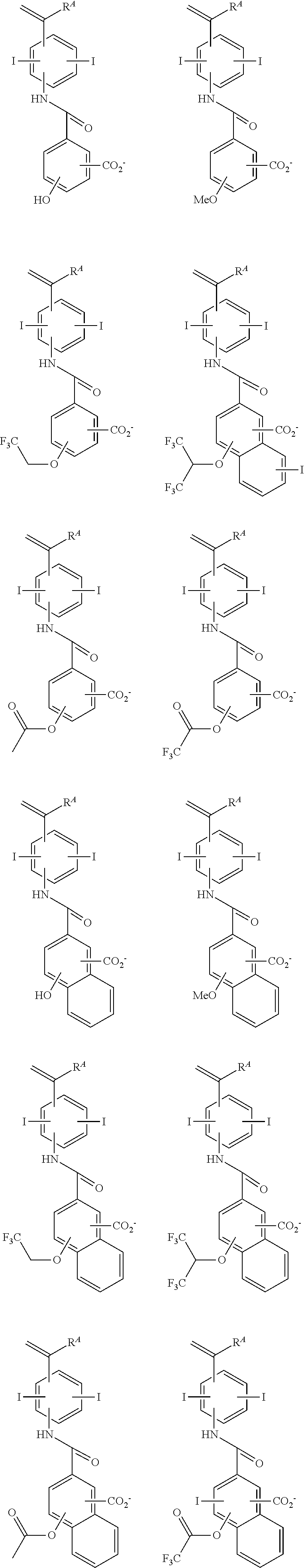

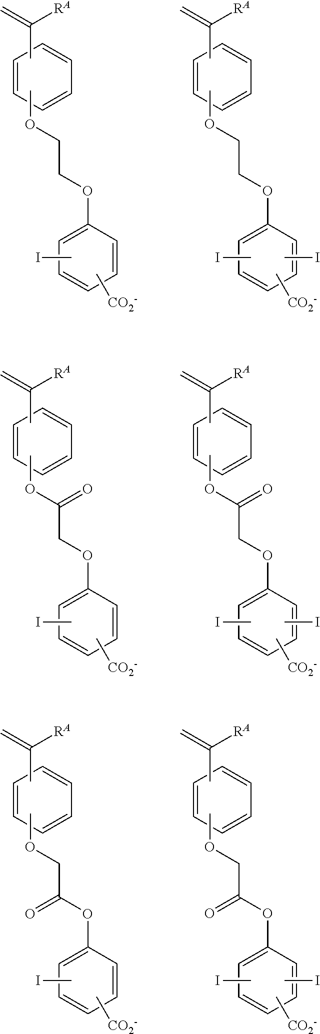

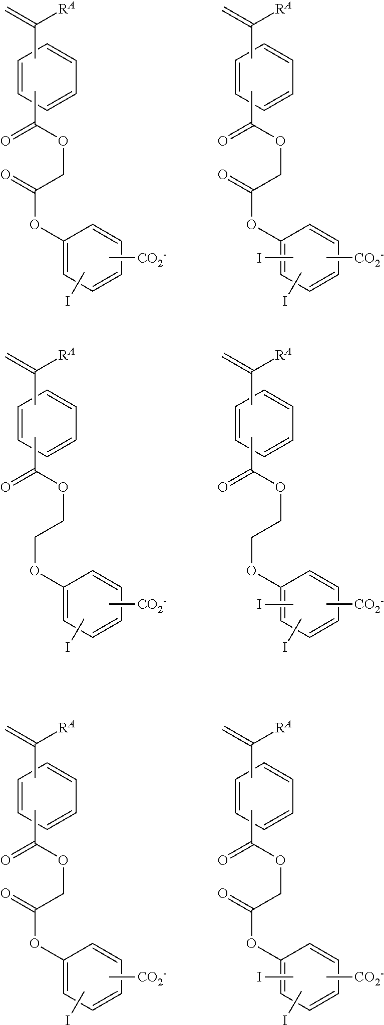

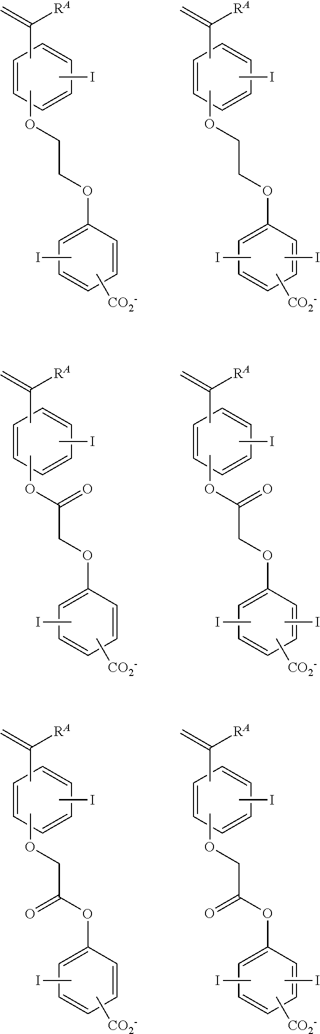

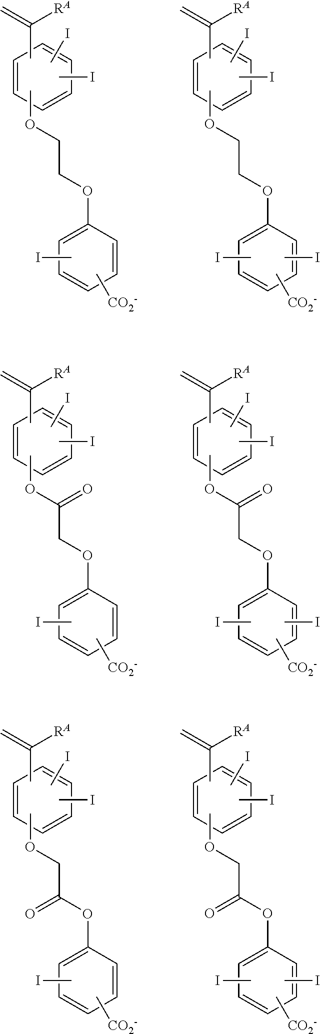

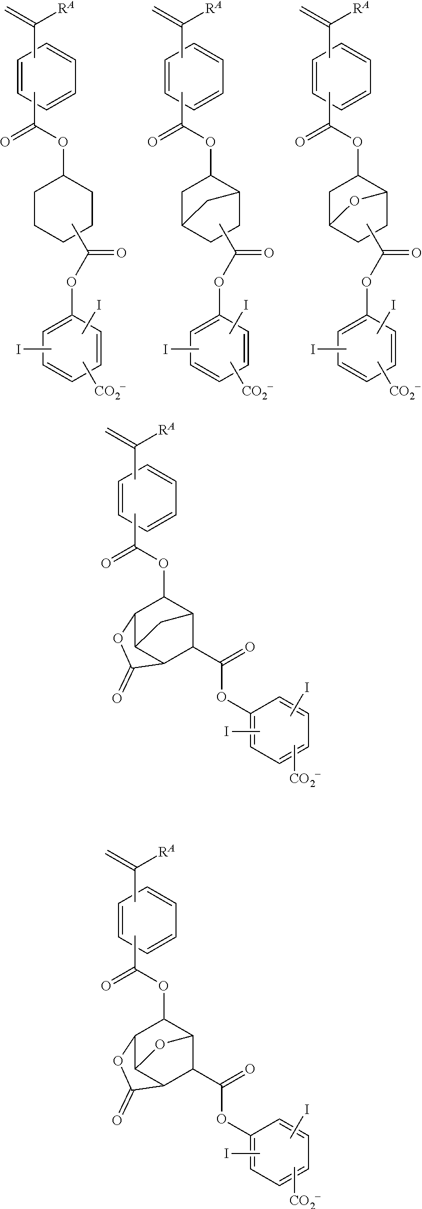

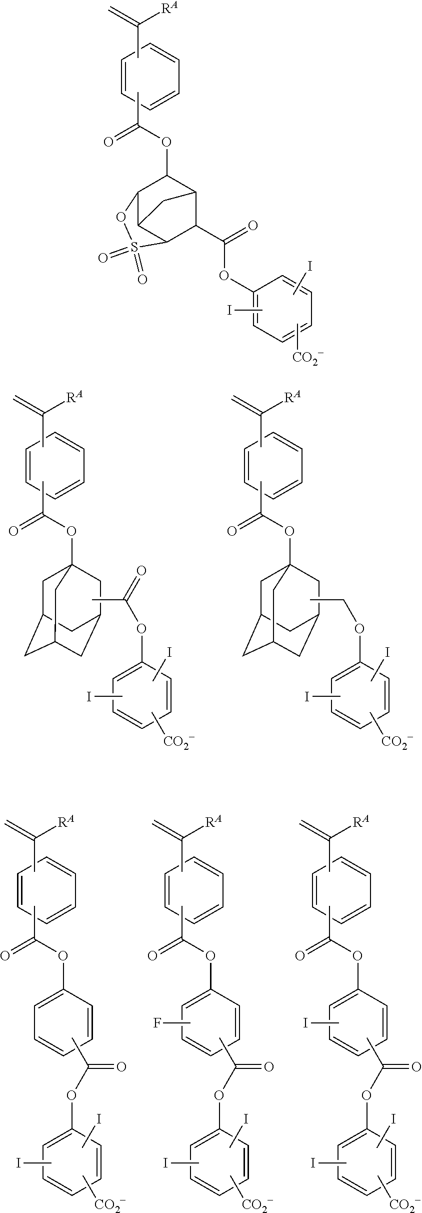

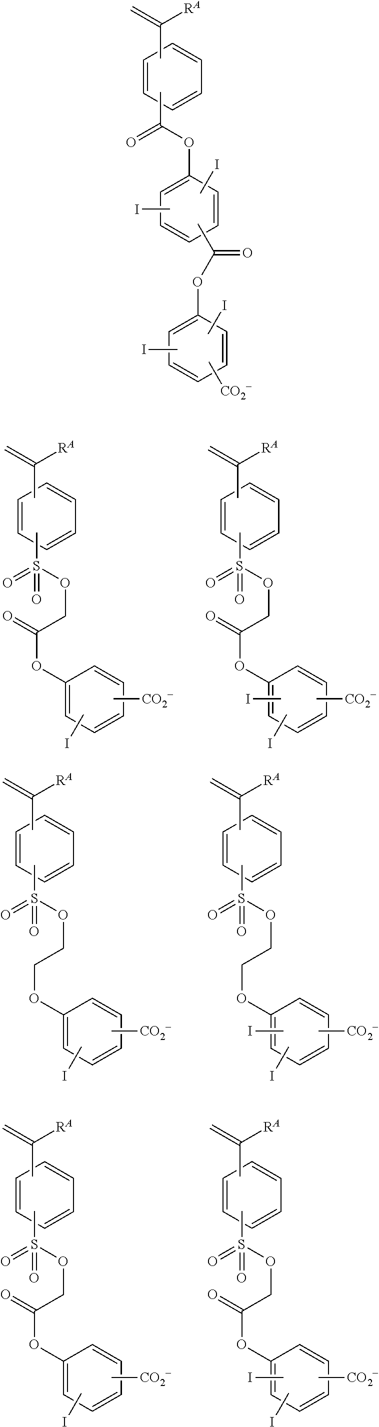

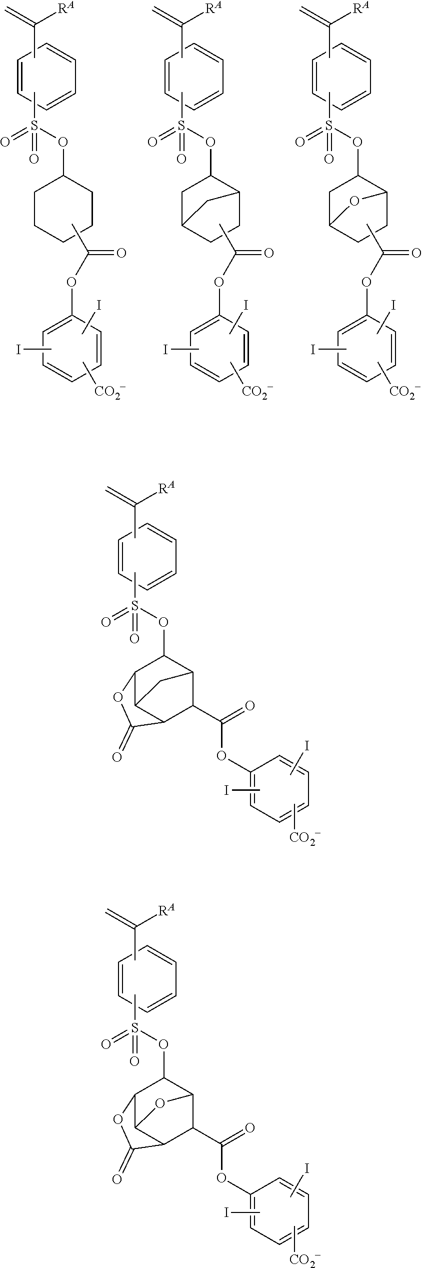

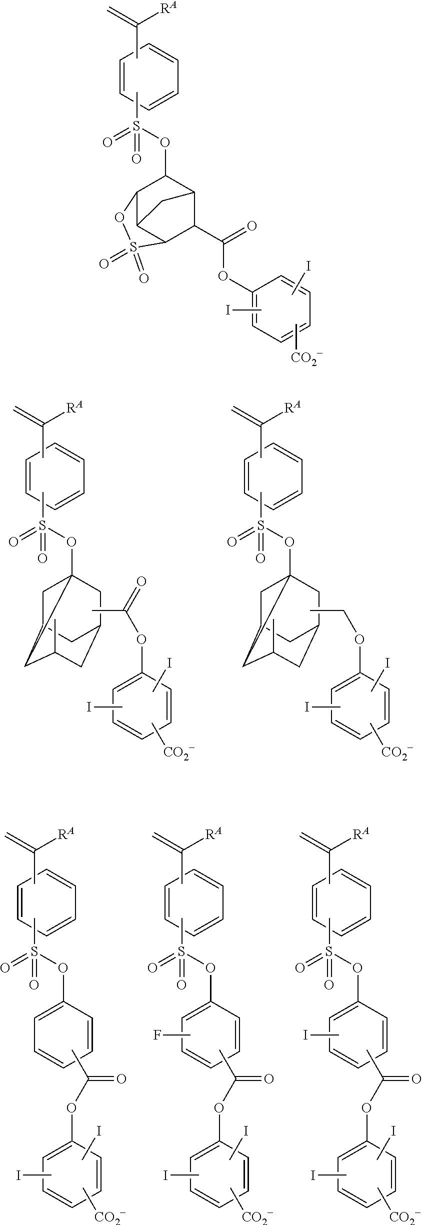

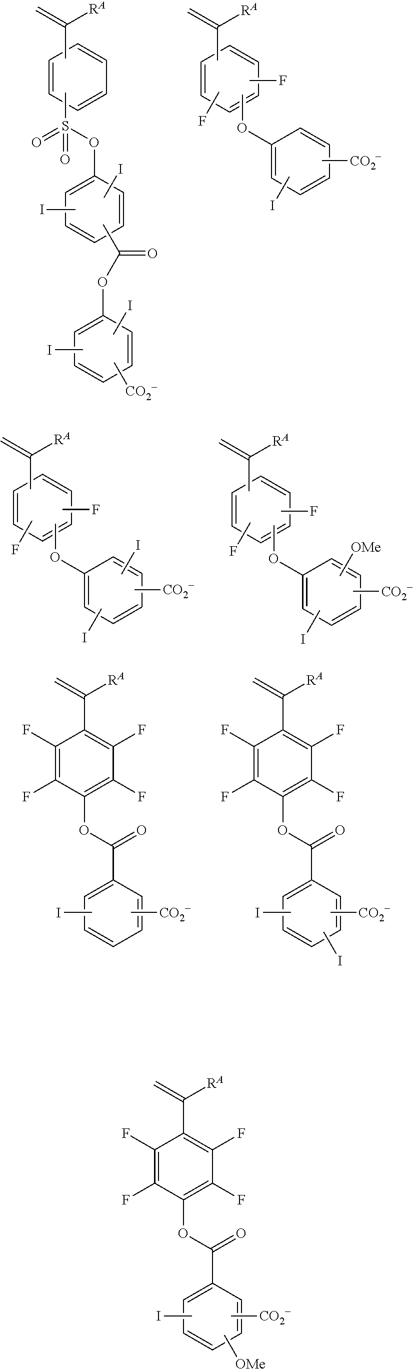

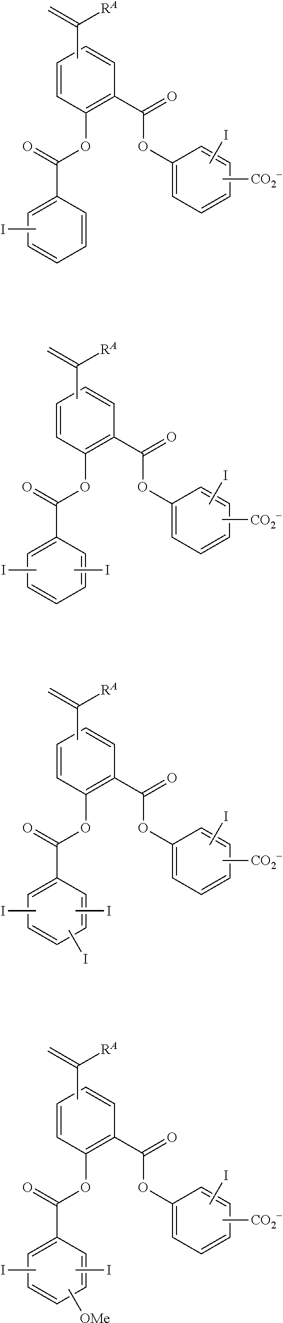

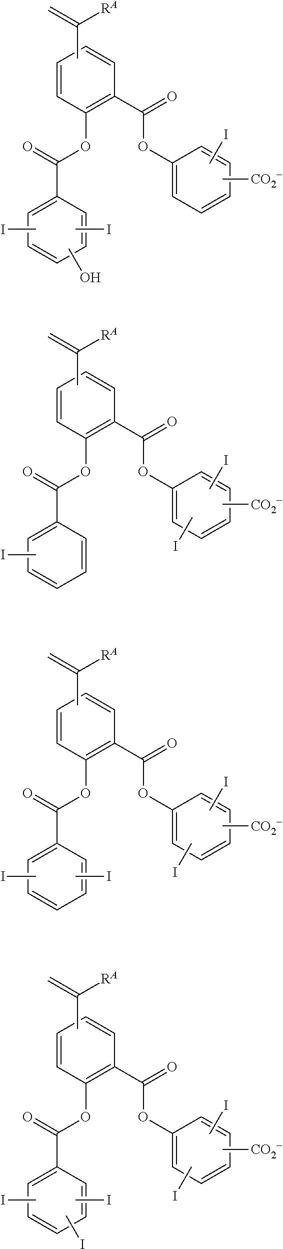

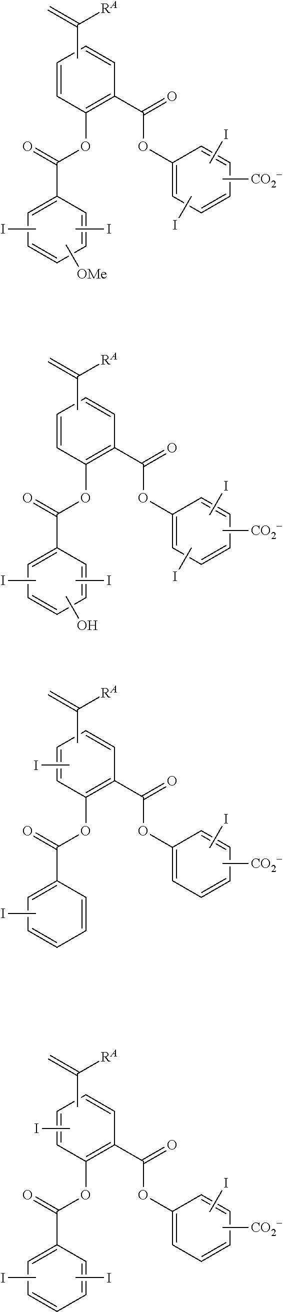

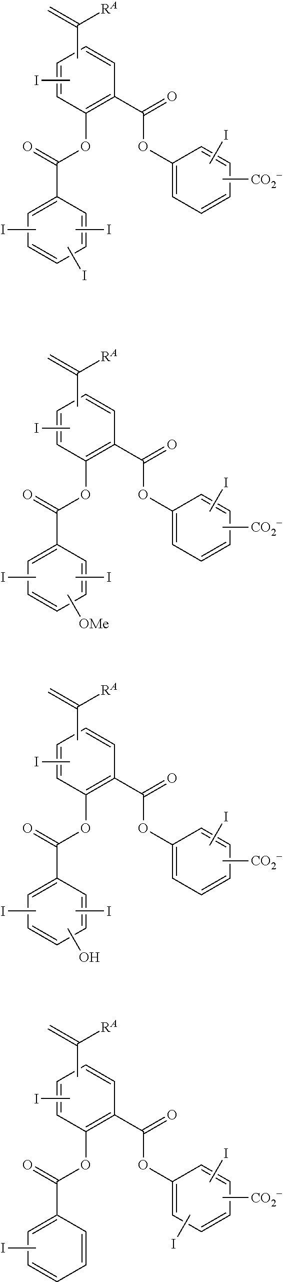

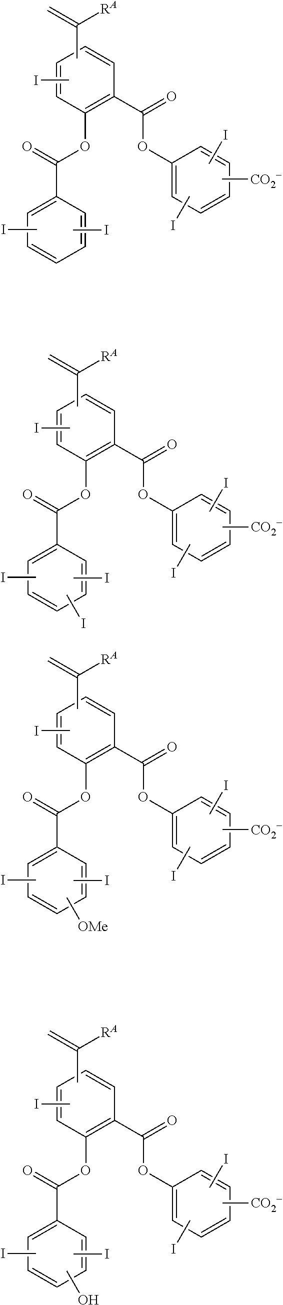

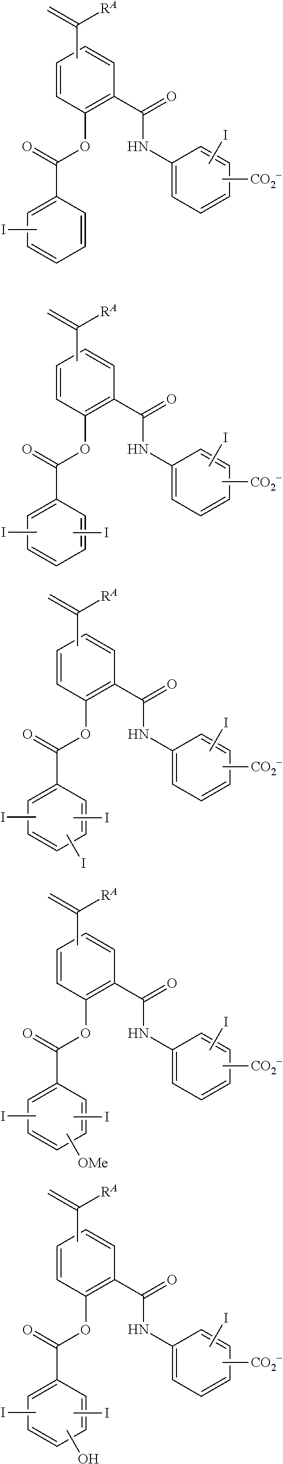

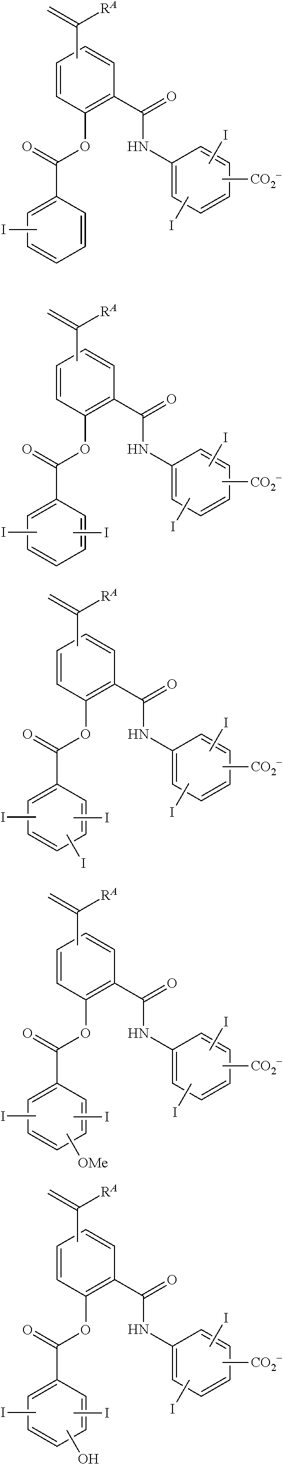

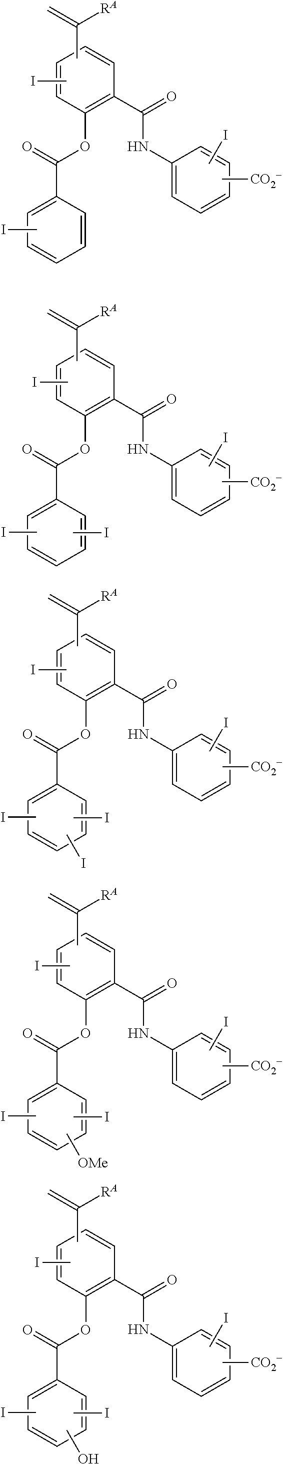

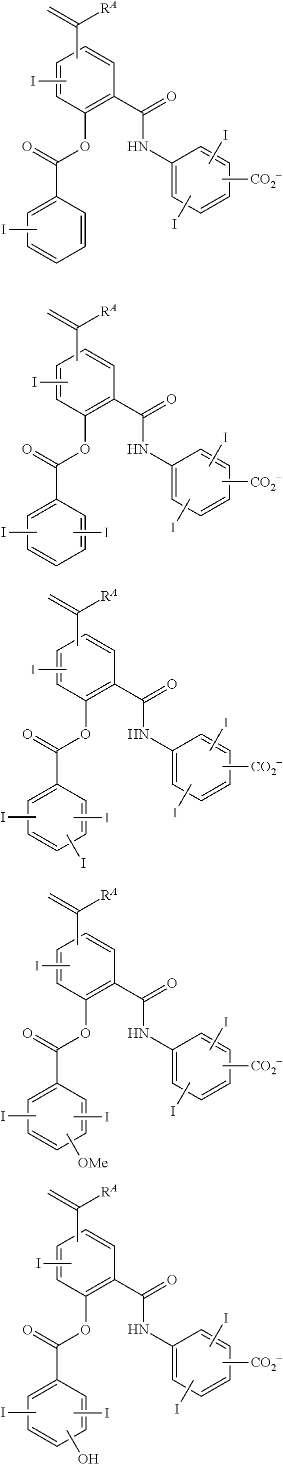

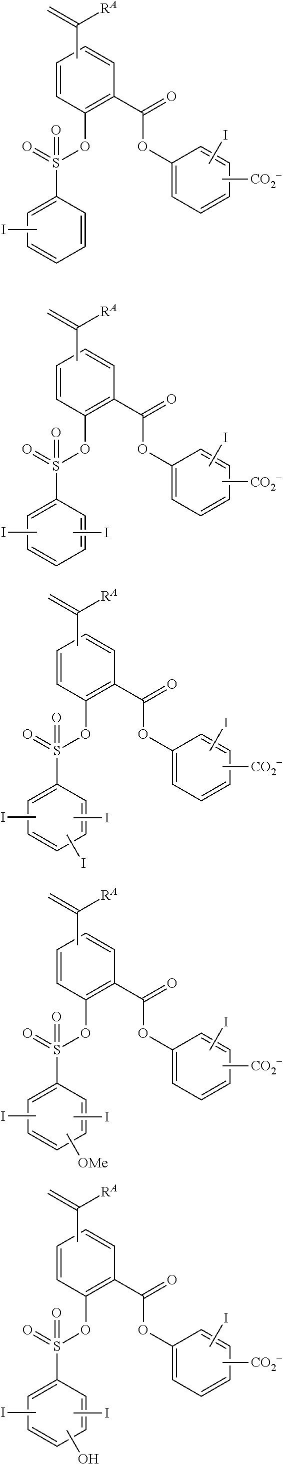

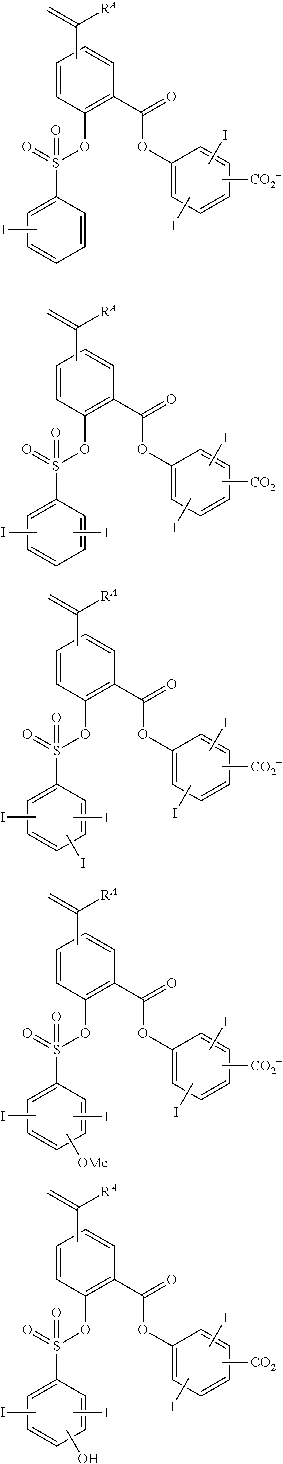

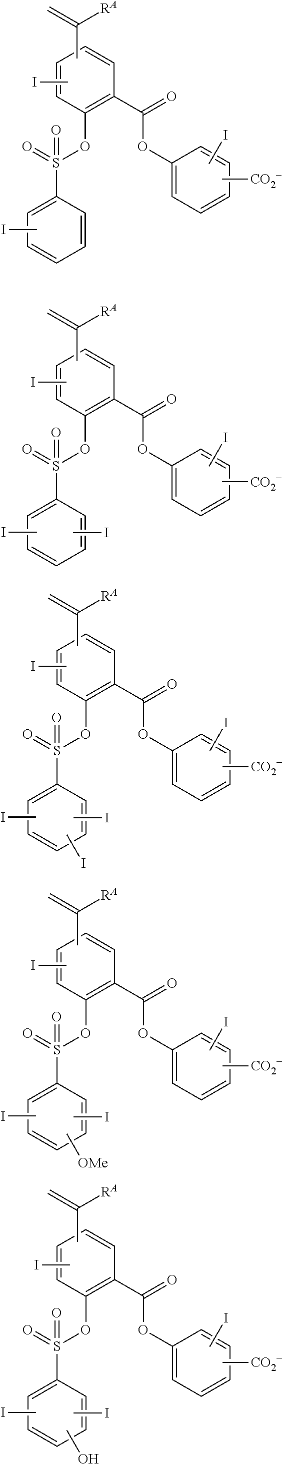

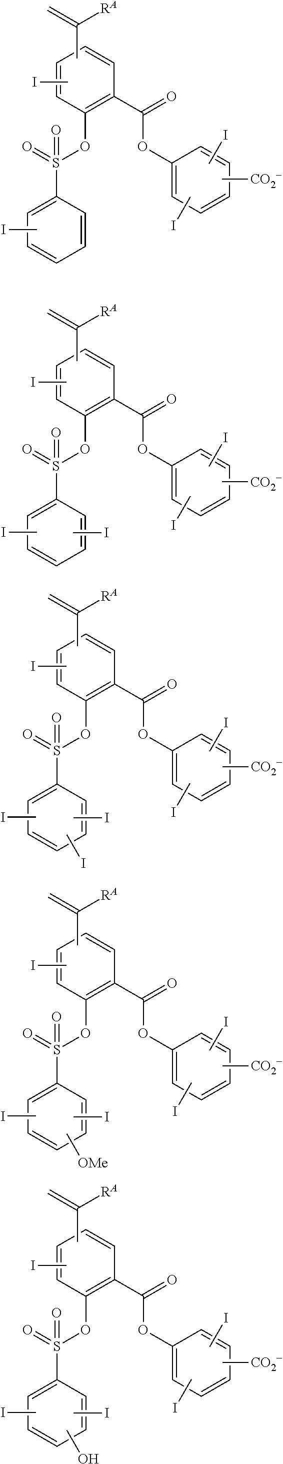

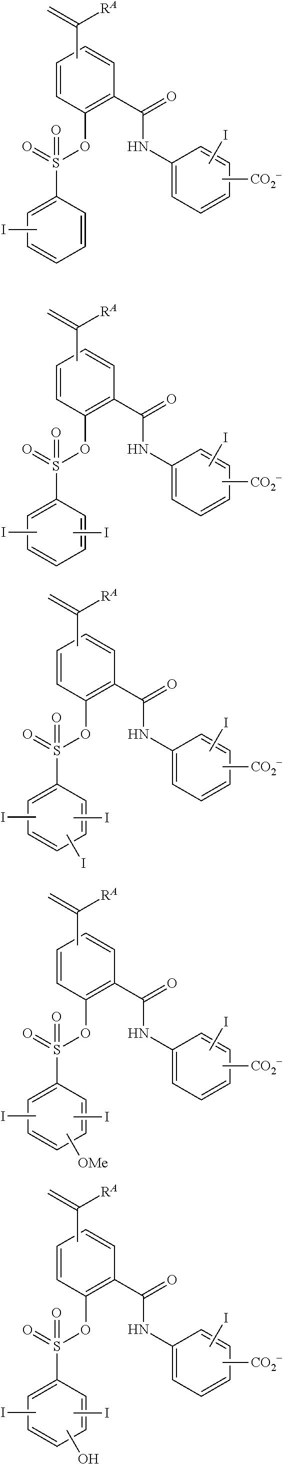

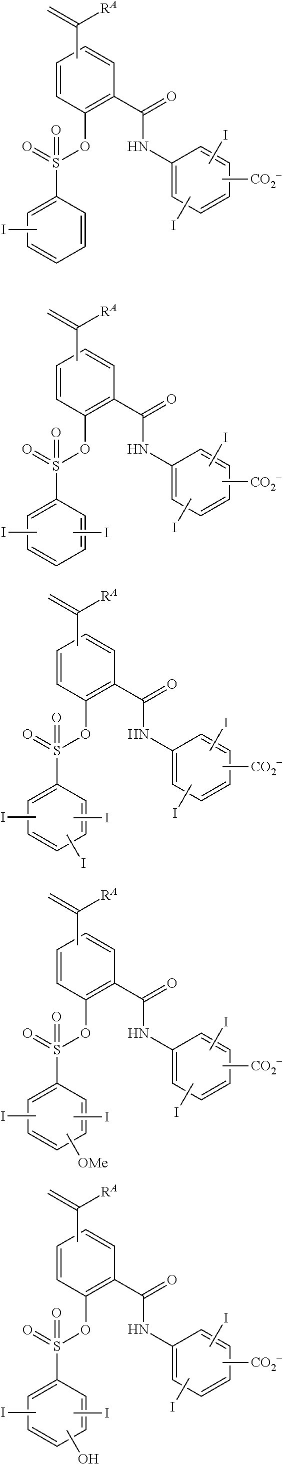

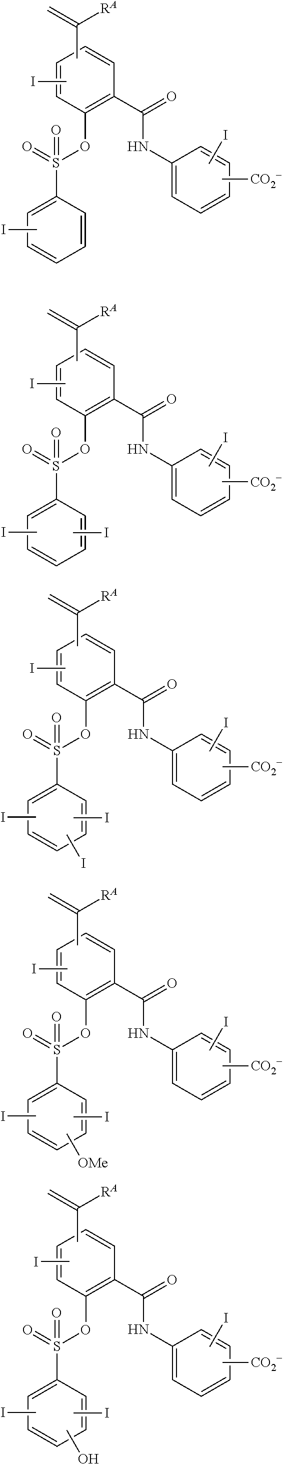

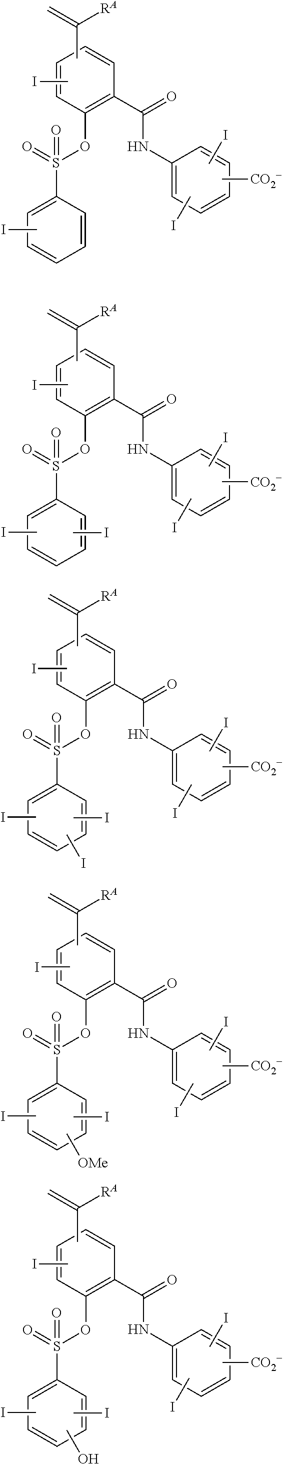

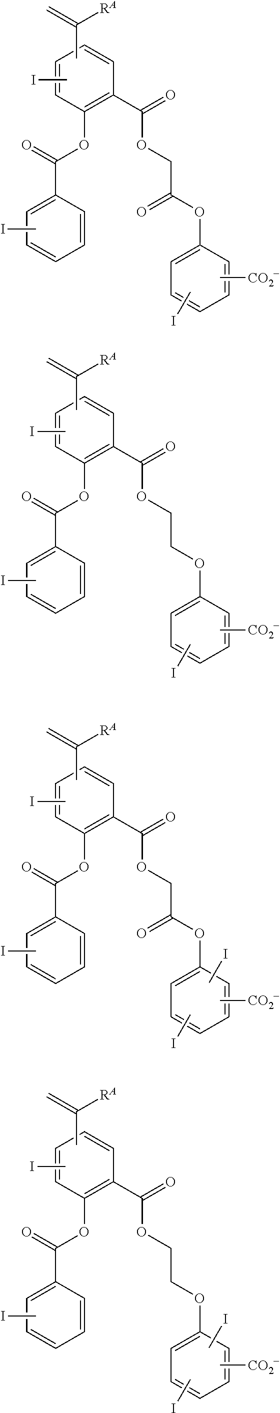

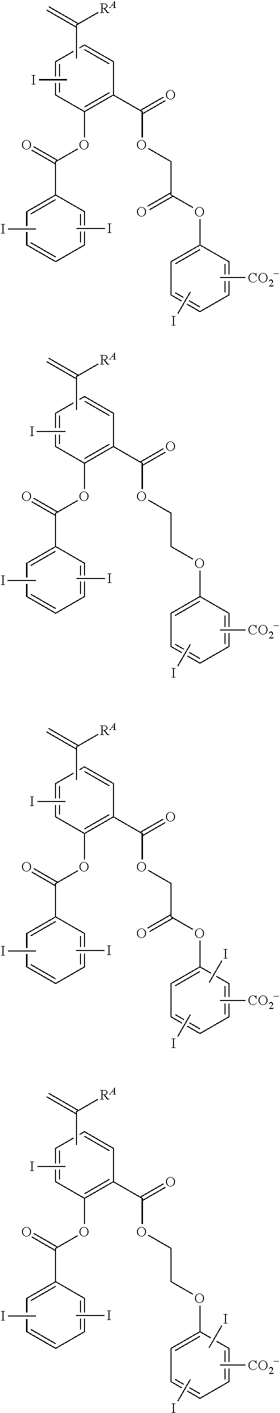

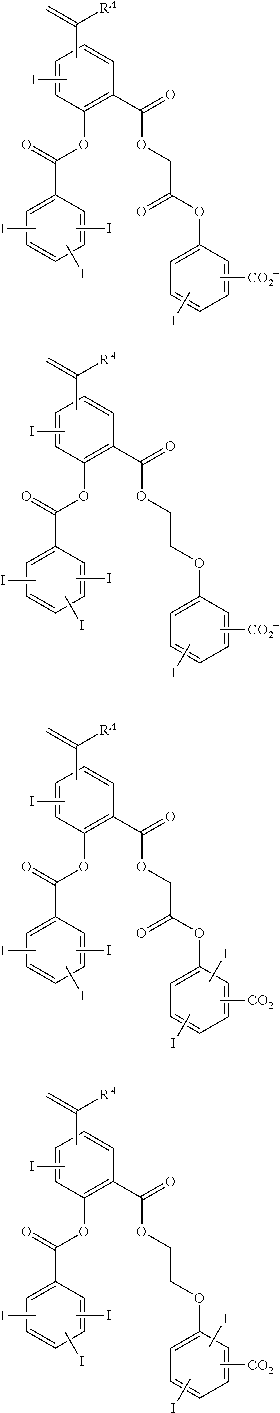

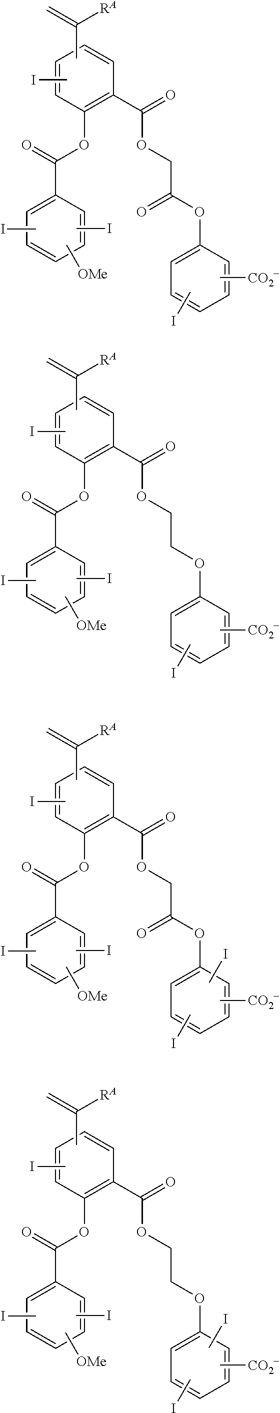

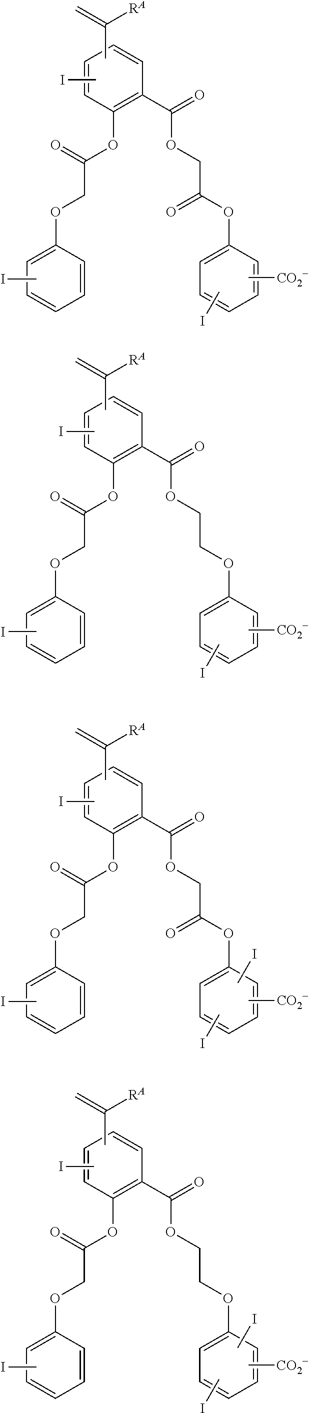

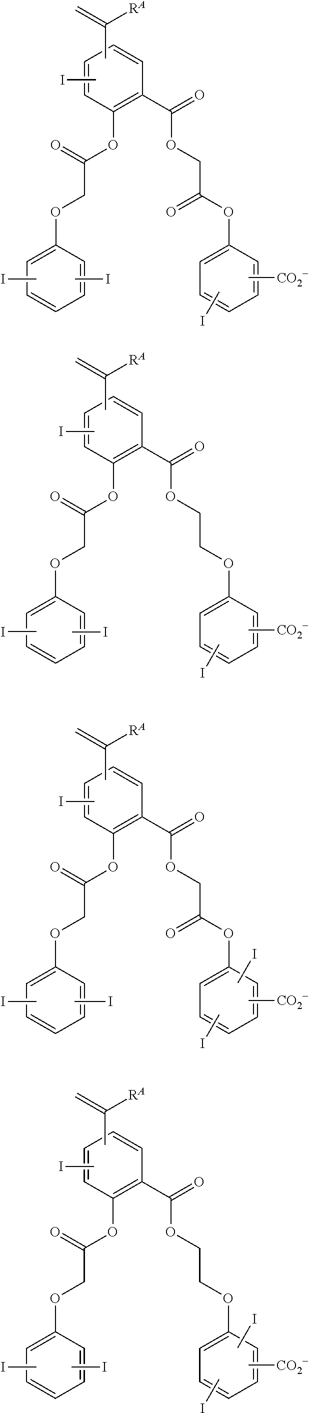

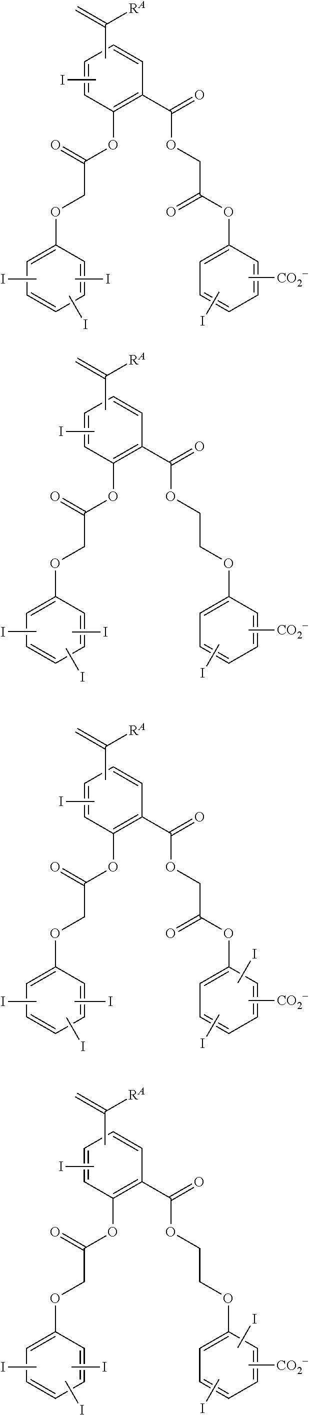

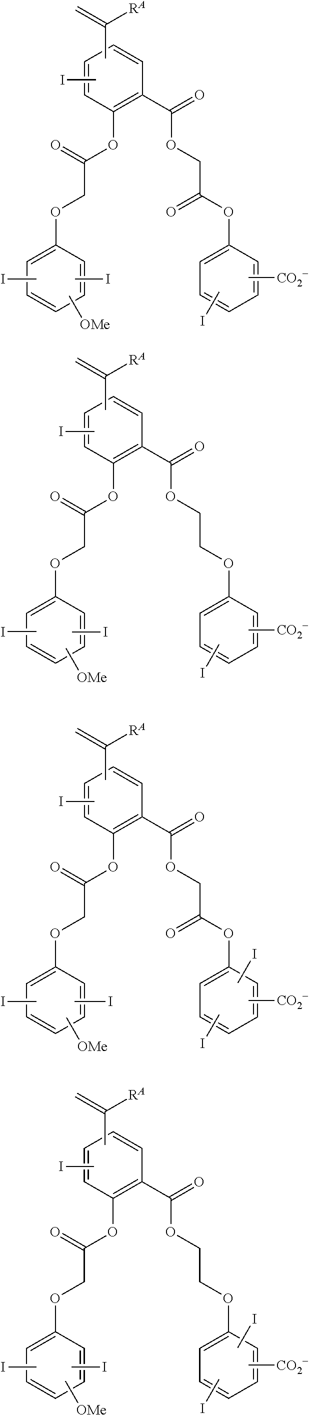

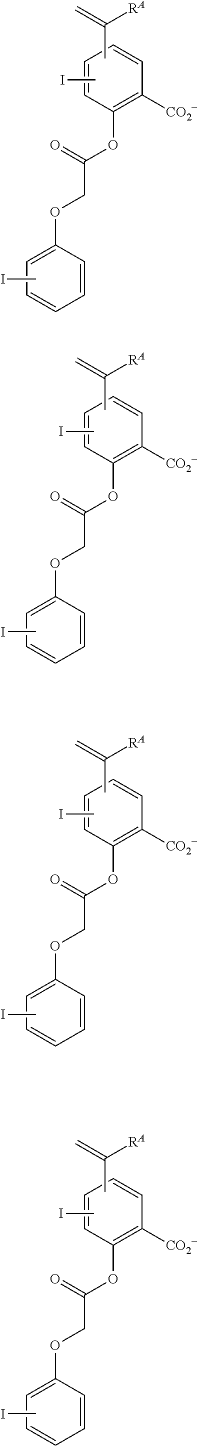

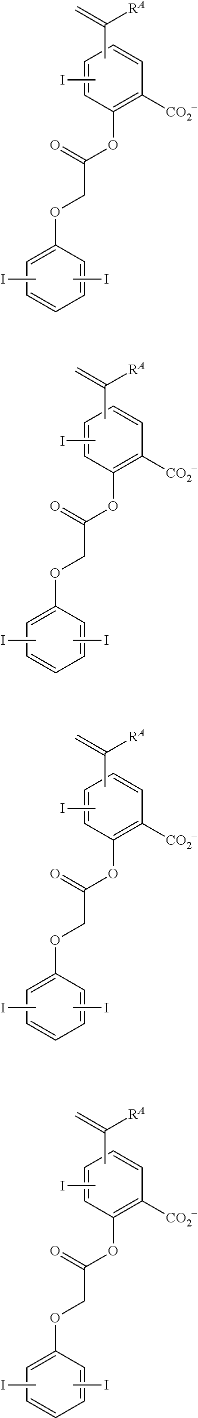

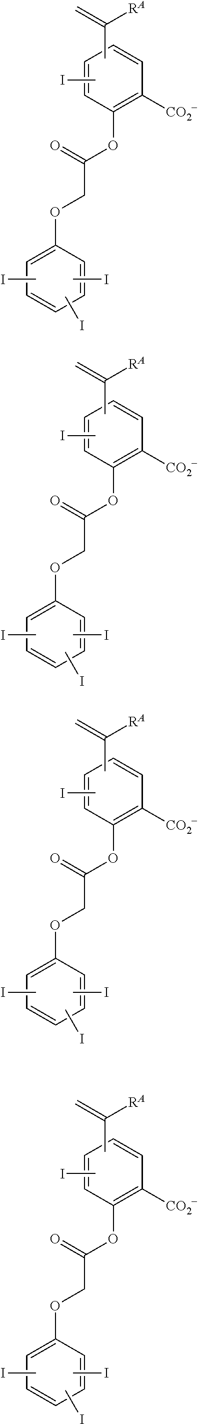

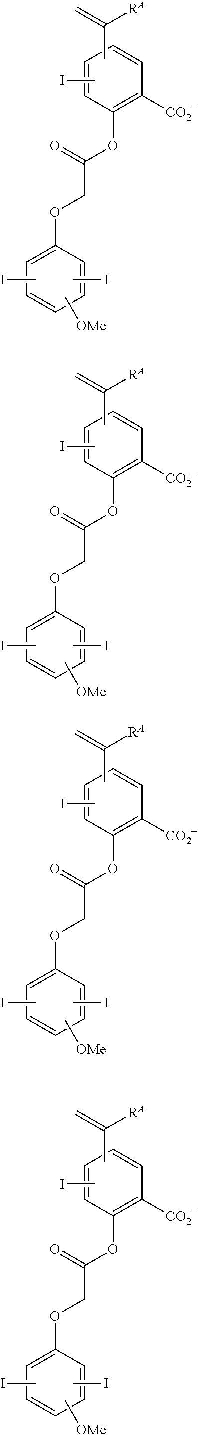

Other examples of the anion in the onium salt monomer having formula (a) are shown in JP 7433394, paragraphs [0049], [0054]-[0056], and [0062]-[0064], but not limited thereto.

Examples of the inventive sulfonium salt monomer include arbitrary combinations of the anion with the cation.

The inventive sulfonium salt monomer can be synthesized by a known method. Examples thereof include methods described in JP 7433394, paragraphs [0402]-[0405], but are not limited thereto.

### Polymer

The inventive polymer comprises repeat units derived from the sulfonium salt monomer having formula (a) (hereinafter, also referred to as repeat units (a)).

The inventive polymer is a polymer-bound quencher that functions as a quencher and also as a base polymer in the chemically amplified resist composition. A structural characteristic of the inventive polymer is that the polymer contains repeat units having a salt structure that is derived from the inventive sulfonium salt monomer, has a benzene or naphthalene structure directly bound to a backbone and contains a carboxylate anion having iodine and a sulfonium cation having a —SF₅ group. Iodine, which is highly absorptive to EUV having a wavelength 13.5 nm, generates secondary electrons upon exposure, and the energy of the secondary electrons is transferred to the acid generator, thereby increasing the sensitivity. A polymerizable group having a styrene or vinylnaphthalene structure has higher rigidity over a polymerizable group such as a methacrylic acid ester, and improves the glass transition temperature (Tg) of the polymer. It is considered that the aromatic rings in the base polymer or between the base polymers interact with each other (exhibits a π-π stacking effect) to regularly arrange the base polymers, and even in fine pattern formation, resistance to pattern collapse is exhibited against the developer. In an etching step after fine pattern formation, excellent etch resistance is also exhibited because the aromatic ring is directly bound to the backbone. As the number of iodine atoms is increased, the solvent solubility decreases, but by making the cation have a —SF₅ group, the solvent solubility can be secured. The —SF₅ group is a strong electron-withdrawing group, which lowers LUMO of the frontier molecular orbit theory by bonding to the aromatic ring of a triaryl sulfonium cation, so that the cation is more likely to accept secondary electrons, whereby decomposition of the sulfonium cation is accelerated. The carboxylate anion is relatively highly basic, so that an acid generated from a strong acid can be effectively trapped. The synergy of these effects increases the sensitivity, improves LWR and CDU, and enables pattern formation in which pattern collapse hardly occurs. Thus, the inventive polymer is particularly suitable as a material for chemically amplified positive resist compositions.

The polymer may further comprise repeat units having the formula (b1) (hereinafter, also referred to as repeat units (b1)) or repeat units having the formula (b2) (hereinafter, also referred to as repeat units (b2)).

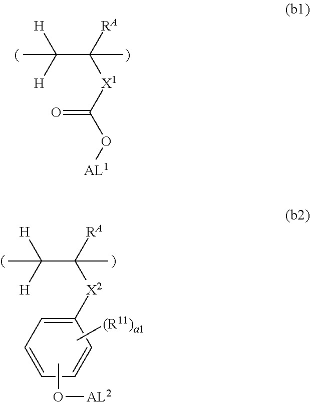

In formulae (b1) and (b2), R^{A} is each independently hydrogen, fluorine, methyl group, or trifluoromethyl group.

In formula (b1), X¹ is a single bond, phenylene group, naphthylene group, *—C(═O)—O—X¹¹— or *—C(═O)—NH—X¹¹—, the phenylene group or naphthylene group may be substituted with hydroxy group, nitro group, cyano group, a C₁-C₁₀ saturated hydrocarbyl group which may contain fluorine, a C₁-C₁₀ saturated hydrocarbyloxy group which may contain fluorine, or halogen. X¹¹ is a C₁-C₁₀ saturated hydrocarbylene group, phenylene group, or naphthylene group, the saturated hydrocarbylene group may contain hydroxy group, ether bond, ester bond or lactone ring, * designates a point of attachment to the carbon atom in the backbone,

In formula (b2), X² is a single bond, *—C(═O)—O— or *—C(═O)—NH—. * designates a point of attachment to the carbon atom in the backbone, R¹¹ is halogen, cyano group, hydroxy group, nitro group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, The subscript a1 is 0, 1, 2, 3 or 4, preferably 0 or 1. A plurality of R¹¹ may be identical or different when a1 is 2 or more,

In formulae (b1) and (b2), AL¹ and AL² are each independently an acid labile group. Examples of the acid labile group are as shown in JP-A 2013-80033 and JP-A 2013-83821, but are not limited thereto.

Typical of the acid labile group are groups having the following formulae (AL-1) to (AL-3).

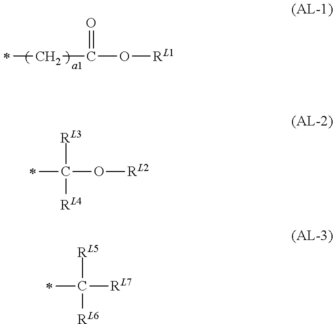

Herein, * designates a point of attachment.

In formulae (AL-1) and (AL-2), R^{L1} and R^{L2} are each independently a C₁-C₄₀ hydrocarbyl group, and may contain a heteroatom such as oxygen, sulfur, nitrogen, fluorine or iodine. The hydrocarbyl group may be saturated or unsaturated, and may be straight, branched, or cyclic. The hydrocarbyl group is preferably a C₁-C₂₀ hydrocarbyl group.

In formula (AL-1), a2 is an integer of 0 to 10, preferably 1, 2, 3, 4 or 5.

In formulae (AL-2), R^{L3} and R^{L4} are each independently hydrogen, a C₁-C₂₀ hydrocarbyl group, and may contain a heteroatom such as oxygen, sulfur, nitrogen, fluorine or iodine. The hydrocarbyl group may be saturated or unsaturated, and may be straight, branched, or cyclic. Two of R^{L2}, R^{L3} and R^{L4} may bond together to form a C₃-C₂₀ ring with the carbon atoms or carbon and oxygen atoms to which they are attached. The ring is preferably a C₄-C₁₆ ring, particularly preferably in an alicyclic form.

In formula (AL-3), R^{L5}, R^{L6} and R^{L7} are each independently a C₁-C₂₀ hydrocarbyl group, and may contain a heteroatom such as oxygen, sulfur, nitrogen, fluorine or iodine. The hydrocarbyl group may be saturated or unsaturated, and may be straight, branched, or cyclic. Two of R^{L5}, R^{L6} and R^{L7} may bond together to form a C₃-C₂₀ ring with the carbon atoms to which they are attached. The ring is preferably a C₄-C₁₆ ring, particularly preferably in an alicyclic form.

Examples of repeat unit (b1) are shown below, but not limited thereto. Herein, R^{A} and AL¹ are as defined above.

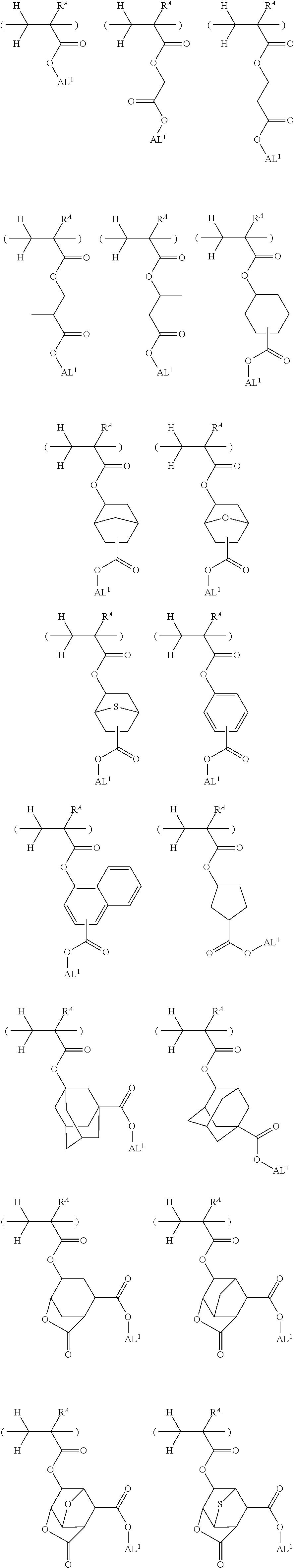

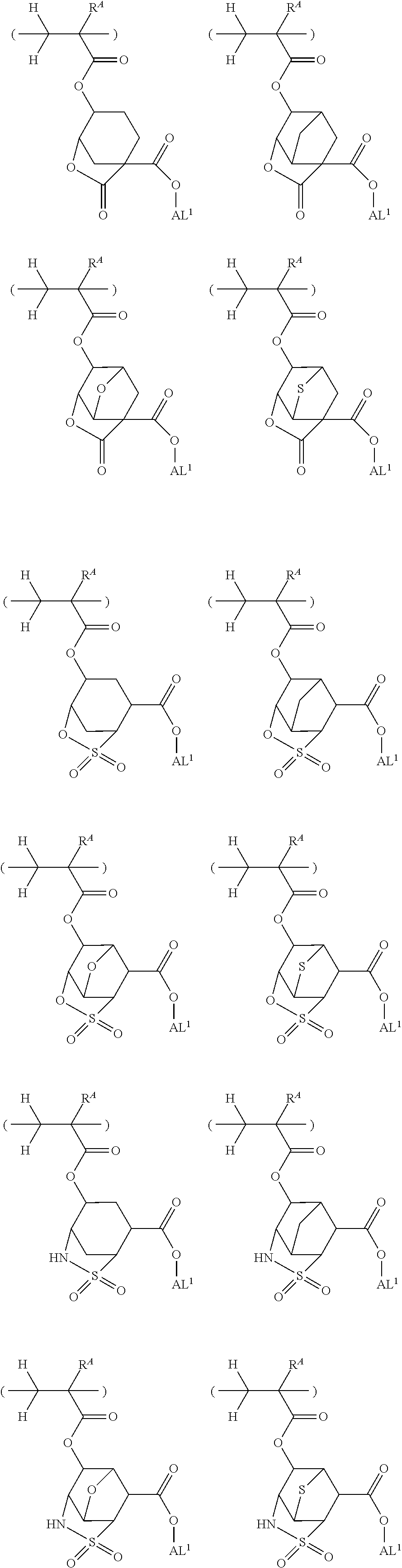

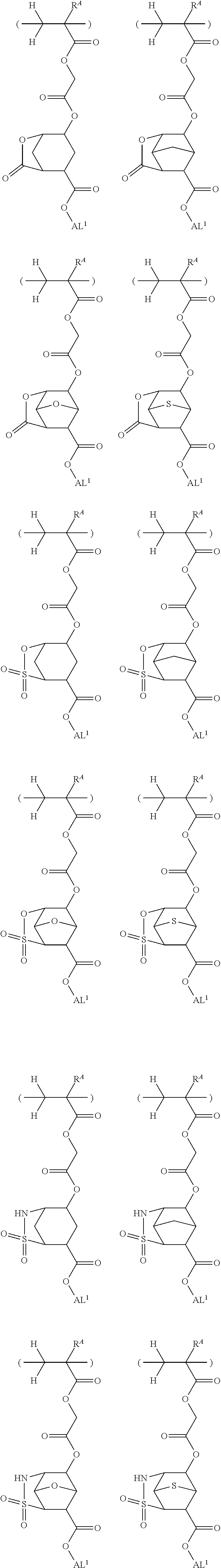

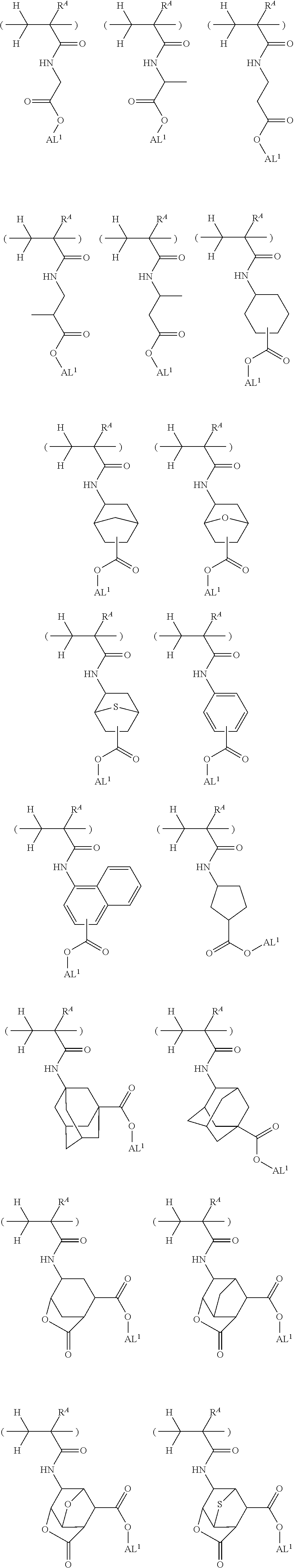

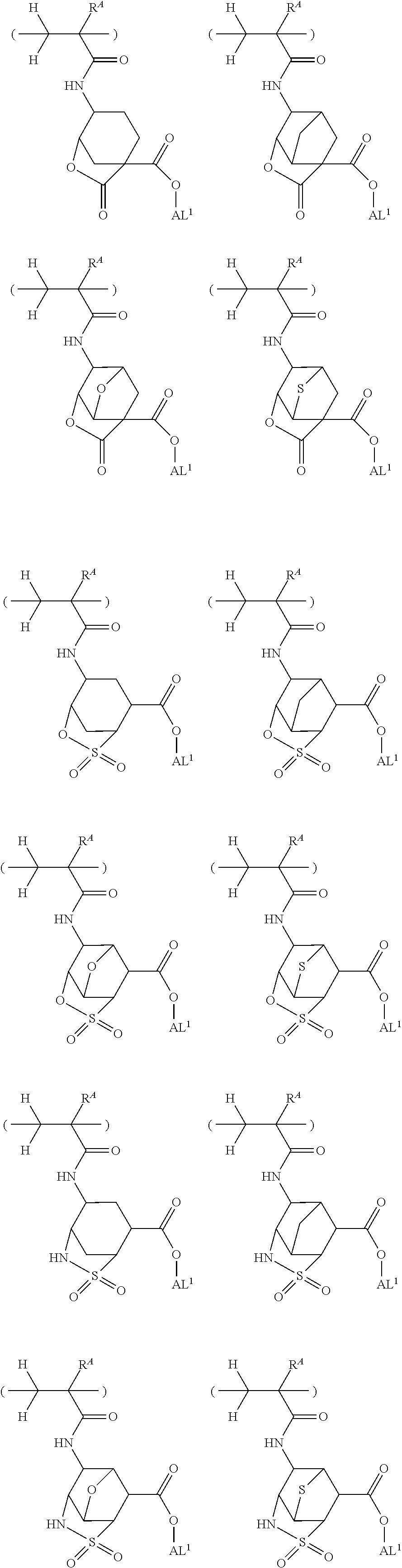

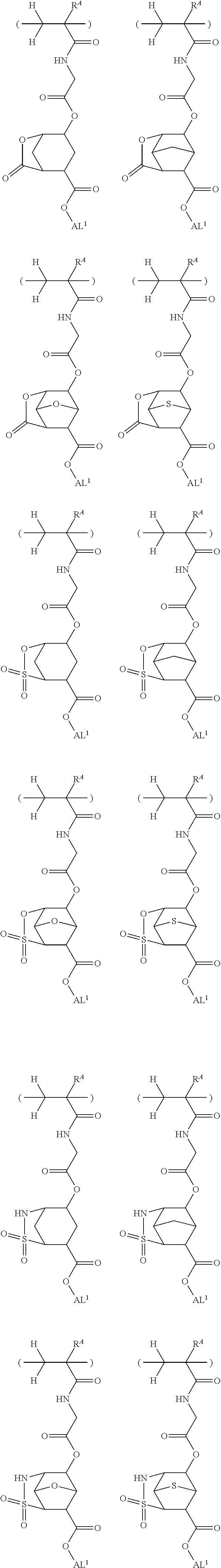

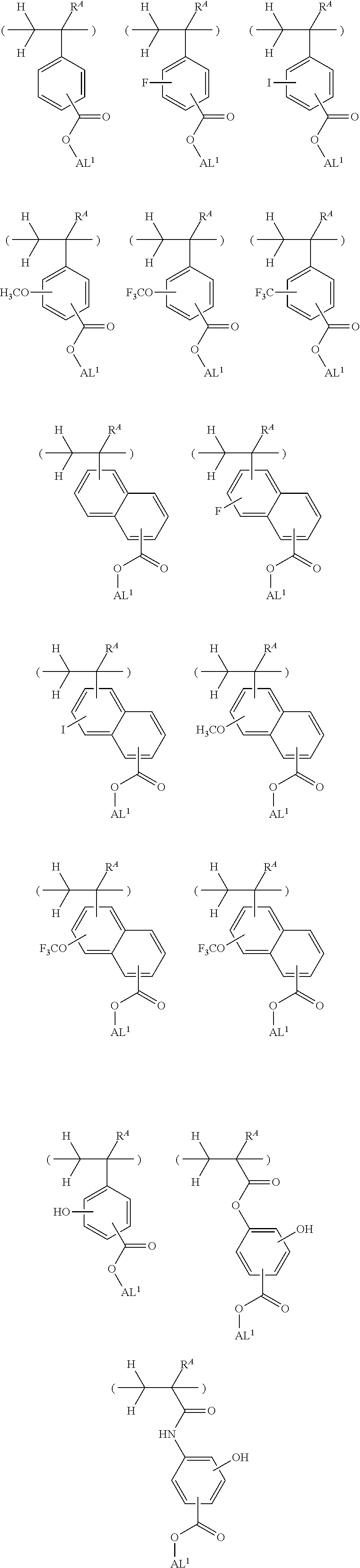

Examples of repeat unit b2 are shown below, but not limited thereto. Herein, R^{A} and AL² are as defined above.

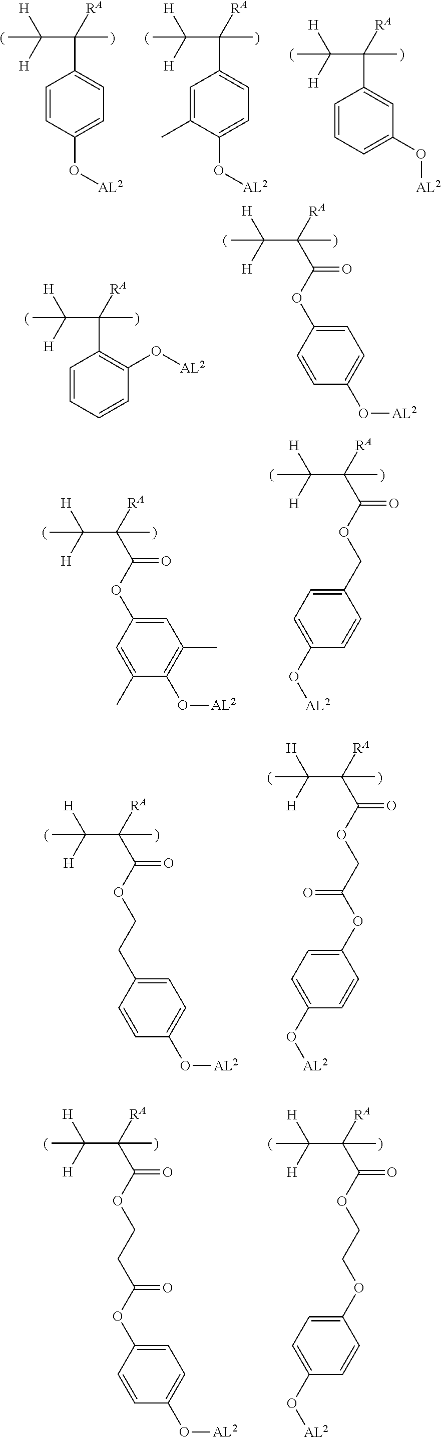

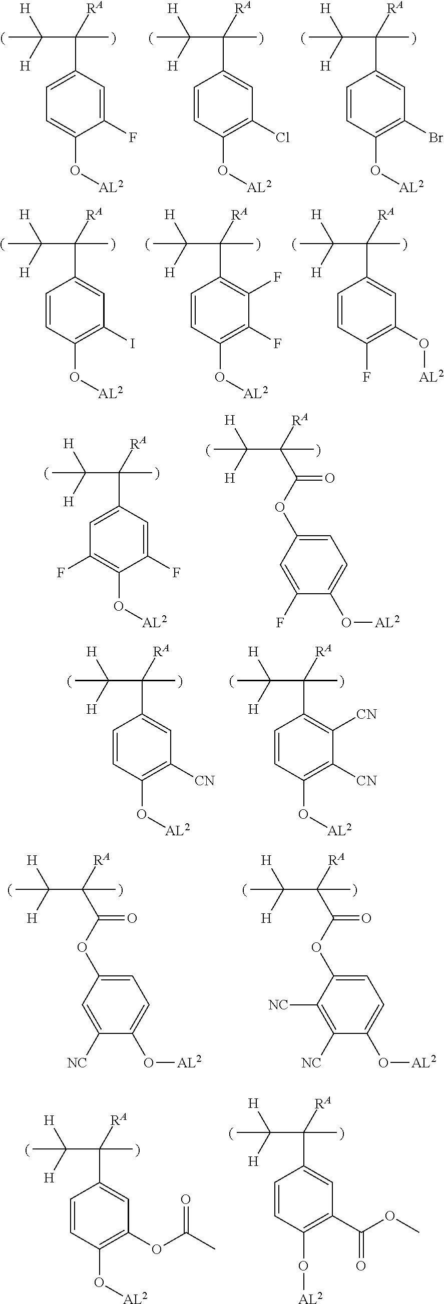

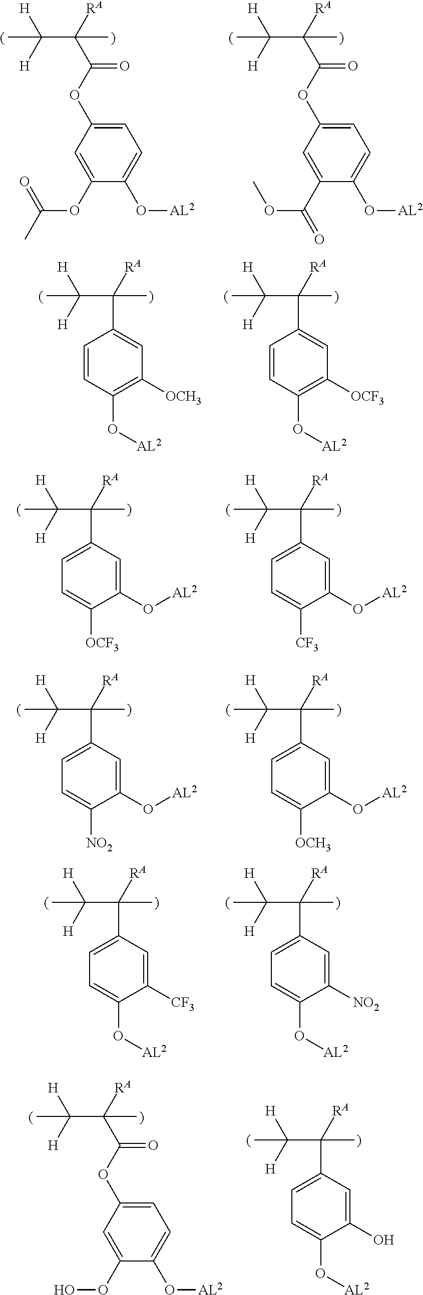

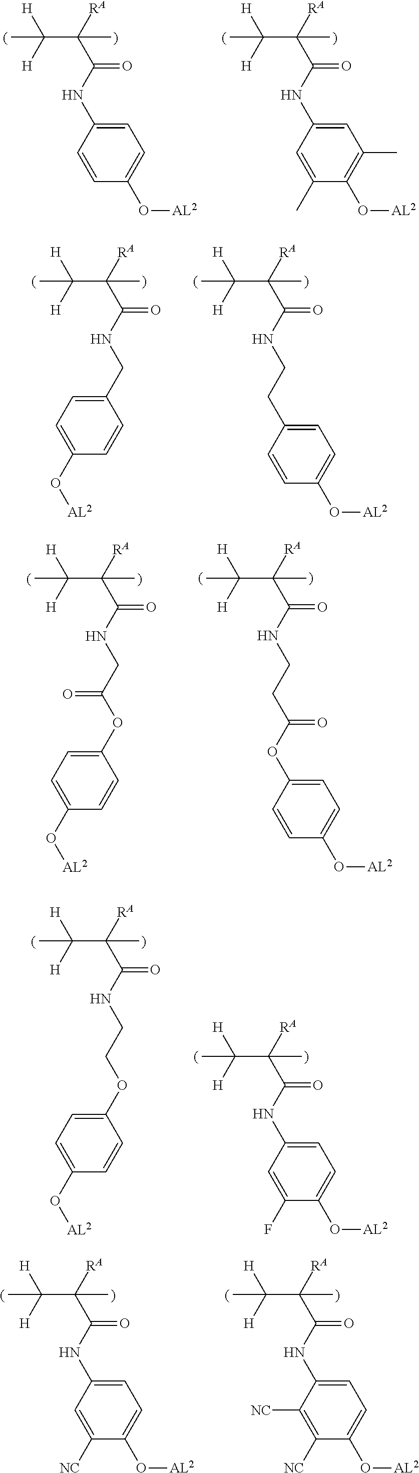

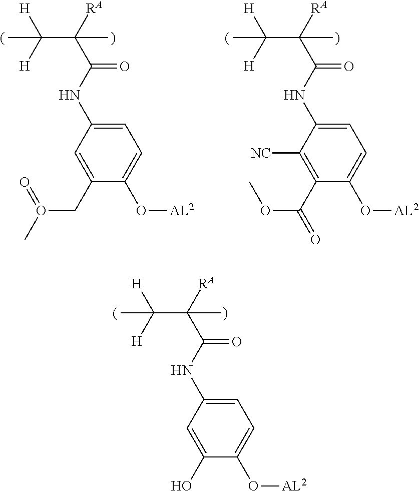

The polymer may further comprise repeat units represented by the formula (b3) (hereinafter also referred to as repeat units (b3)).

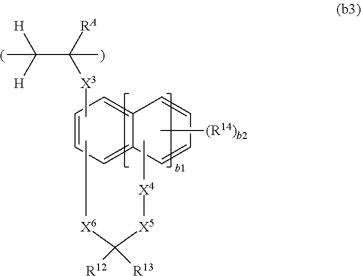

In formula (b3), b1 is 0 or 1. The sulfonium salt monomer has a benzene ring when b1 is 0, and a naphthalene ring when b1 is 1, and b1 is preferably 0 from the aspect of solvent solubility. b2 is 0, 1, 2 or 3 when b1 is 0, b2 is 0, 1, 2, 3, 4 or 5 when b1 is 1. From the aspect of reactant availability, b2 is preferably 0, 1, 2 or 3, more preferably 0, 1 or 2.

In formula (b3), R^{A} is hydrogen, fluorine, a methyl group, or a trifluoromethyl group. Of these, hydrogen and methyl group are preferred, and hydrogen is more preferred.

In formula (b3), X³ is a single bond, *—C(═O)—O— or *—C(═O)—NH—. * designates a point of attachment to the carbon atom in the backbone, Of these, a single bond and *—C(═O)—O— are preferred, and a single bond is more preferred.

In formula (b3), R¹² and R¹³ are each independently halogen or a C₁-C₂₀ hydrocarbyl group which may contain a heteroatom. The hydrocarbyl group may be saturated or unsaturated, and may be straight, branched, or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl and icocyl groups; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl and adamantyl groups; C₂-C₂₀ alkenyl groups such as vinyl, allyl, propenyl, butenyl and hexenyl groups; C₃-C₂₀ cyclic unsaturated hydrocarbyl groups such as a cyclohexenyl group; C₂-C₂₀ aryl groups such as phenyl and naphthyl groups; C₇-C₂₀ aralkyl groups such as benzyl, 1-phenylethyl and 2-phenylethyl groups; and combinations thereof. Some or all of hydrogen atoms of the hydrocarbyl group may be replaced by a group containing a heteroatom such as oxygen, sulfur, nitrogen or halogen, some constituent —CH₂— of the hydrocarbyl group may be replaced by a group containing a heteroatom such as oxygen, sulfur or nitrogen, and as a result, the hydrocarbyl group may contain a hydroxy group, a cyano group, fluorine, chlorine, bromine, iodine, a carbonyl group, an ether bond, an ester bond, a sulfonic ester bond, a carbonate bond, a lactone ring, a sultone ring, carboxylic anhydride (—C(═O)—O—C(═O)—), a haloalkyl group, or the like.

In addition, R¹² and R¹³ are optionally bonded to each other to form a ring together with a nitrogen atom to which these groups are bonded. Examples of the ring formed herein include cyclopropane, cyclobutane, cyclopentane, cyclohexane, norbornane and adamantane rings. Some or all of hydrogen atoms in the ring may be replaced by a group containing a heteroatom such as oxygen, sulfur, nitrogen or halogen, some constituent —CH₂— in the ring may be replaced by a group containing a heteroatom such as oxygen, sulfur or nitrogen, and as a result, the hydrocarbyl group may contain a hydroxy group, fluorine, chlorine, bromine, iodine, a cyano group, a carbonyl group, an ether bond, an ester bond, a sulfonic ester bond, a carbonate bond, a lactone ring, a sultone ring, carboxylic anhydride (—C(═O)—O—C(═O)—), a haloalkyl group, or the like.

In formula (b3), R¹⁴ is halogen, hydroxy group, cyano group, nitro group, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{14A})(R^{14B}). R^{14A} and R^{14B} are each independently hydrogen or a C₁-C₆ hydrocarbyl group, The halogen is preferably fluorine, chlorine, bromine or iodine, more preferably fluorine or iodine. The hydrocarbyl group and hydrocarbyl moiety of the hydrocarbyloxy, hydrocarbyloxycarbonyl and hydrocarbylthio groups may be saturated or unsaturated and straight, branched or cyclic. Examples thereof are as exemplified above as hydrocarbyl groups R¹² and R¹³. Some or all of hydrogen atoms of the hydrocarbyl group may be replaced by a group containing a heteroatom such as oxygen, sulfur, nitrogen or halogen, some constituent —CH₂— of the hydrocarbyl group may be replaced by a group containing a heteroatom such as oxygen, sulfur or nitrogen, and as a result, the hydrocarbyl group may contain a hydroxy group, a cyano group, fluorine, chlorine, bromine, iodine, a carbonyl group, an ether bond, an ester bond, a sulfonic ester bond, a carbonate bond, a lactone ring, a sultone ring, carboxylic anhydride (—C(═O)—O—C(═O)—), a haloalkyl group, or the like. A plurality of R¹⁴ may be identical or different when b2 is 2 or more.

A plurality of R¹⁴ may bond together to form a ring with the aromatic ring carbon atom to which they are attached when b2 is 2 or more. Examples of the ring formed herein include cyclopropane, cyclobutane, cyclopentane, cyclohexane, norbornane and adamantane rings. Some or all of hydrogen atoms in the ring may be replaced by a group containing a heteroatom such as oxygen, sulfur, nitrogen or halogen, some constituent —CH₂— in the ring may be replaced by a group containing a heteroatom such as oxygen, sulfur or nitrogen, and as a result, the hydrocarbyl group may contain a hydroxy group, fluorine, chlorine, bromine, iodine, a cyano group, a carbonyl group, an ether bond, an ester bond, a sulfonic ester bond, a carbonate bond, a lactone ring, a sultone ring, carboxylic anhydride (—C(═O)—O—C(═O)—), a haloalkyl group, or the like.

In formula (b3), X⁴ is a single bond, C₁-C₄ aliphatic hydrocarbylene group, carbonyl group, sulfonyl group or a group obtained by combining the foregoing. Inter alia, a single bond, carbonyl group or sulfonyl group is preferred from the aspect of reactant availability, and a single bond or carbonyl group is more preferred from polar groups formed after the reaction.

In formula (b3), X⁵ and X⁶ are each independently oxygen or sulfur. The moieties X⁴ and X⁶ are attached to adjacent carbon atoms on the aromatic ring. The moieties X⁵ and X⁶ may identical or different, and each of X⁵ and X⁶ is preferably oxygen from the aspect of reactivity.

Examples of repeat units (b3) are shown below, but not limited thereto. In the following formulae, R^{A} is as defined above, and Me is a methyl group. The bonding positions of the substituents on the aromatic ring may be interchanged.

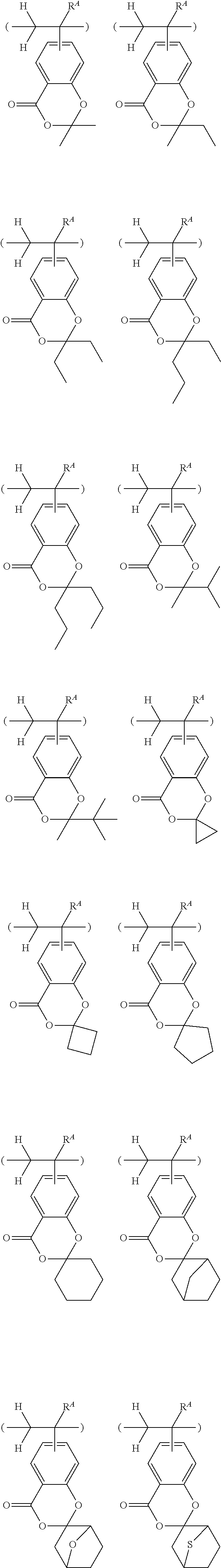

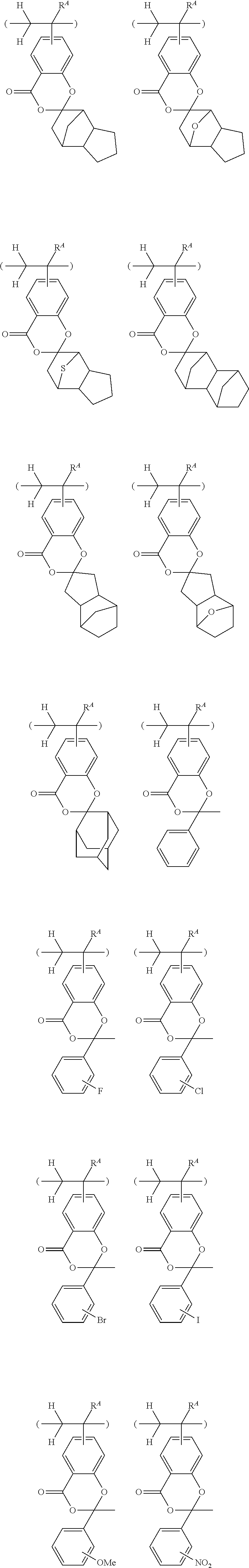

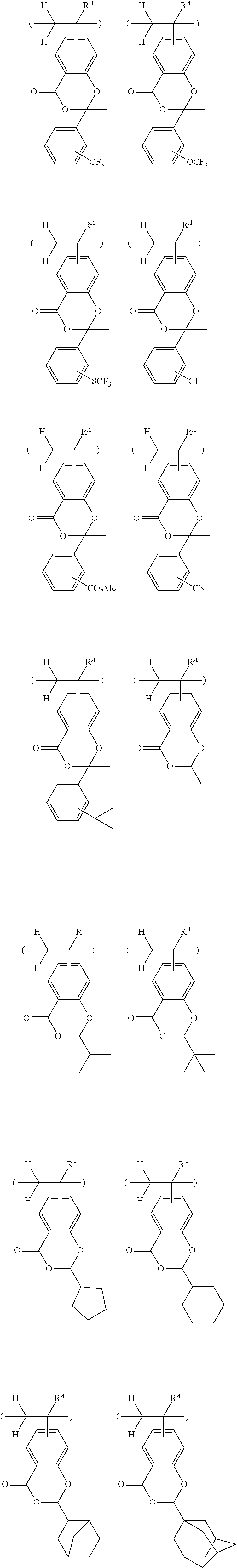

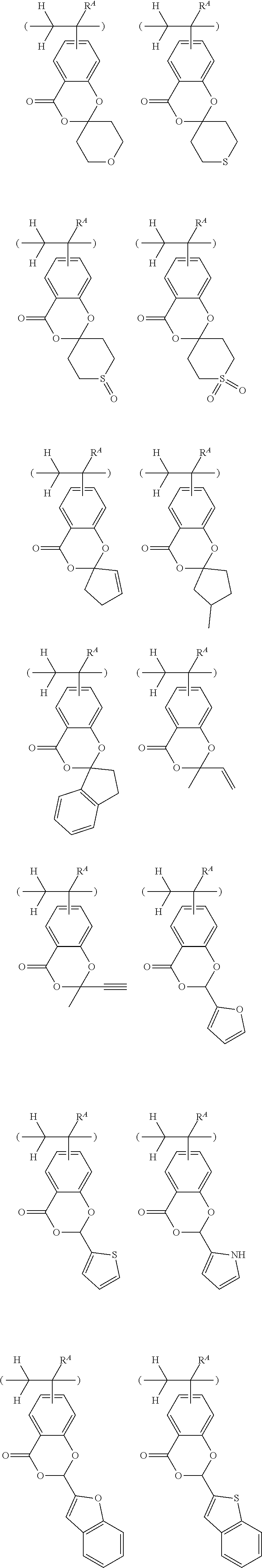

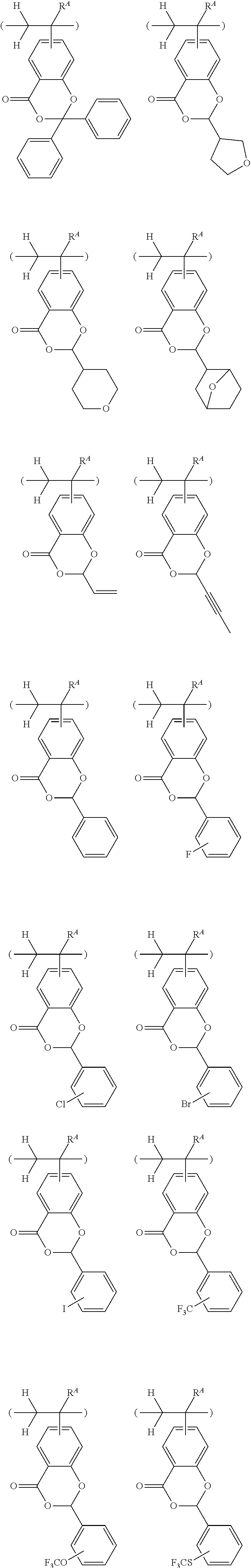

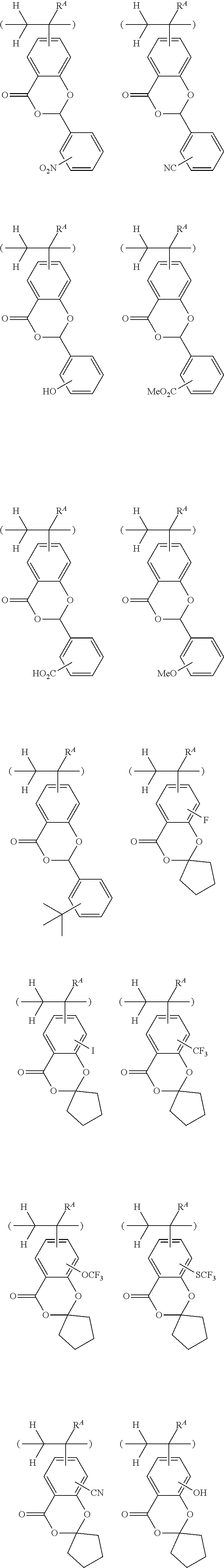

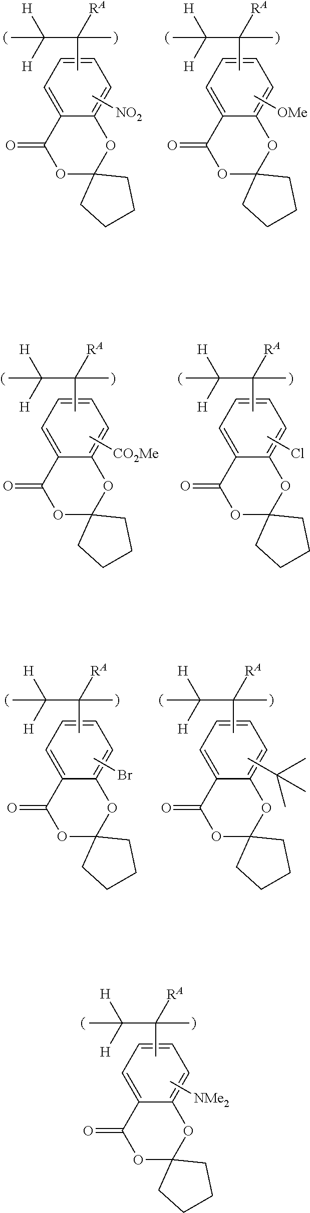

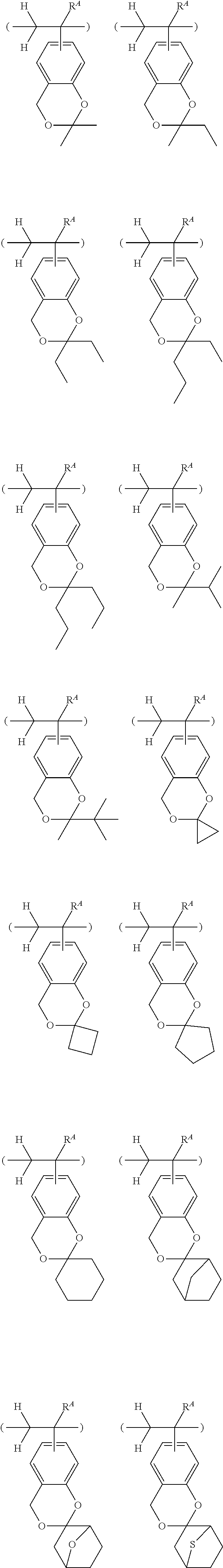

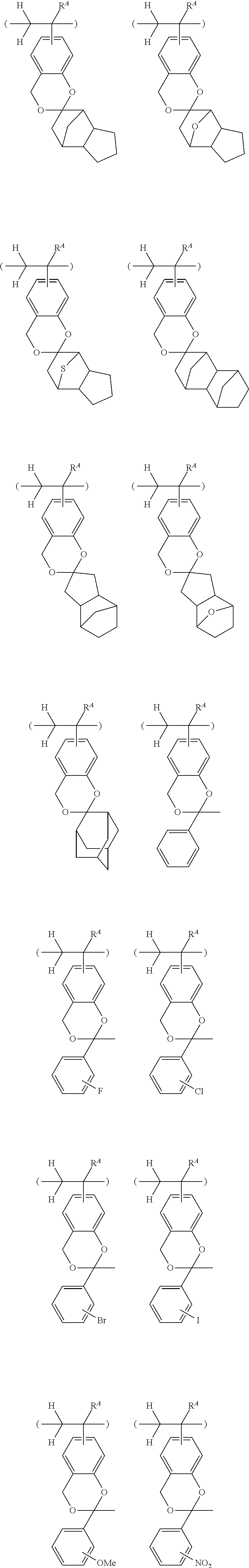

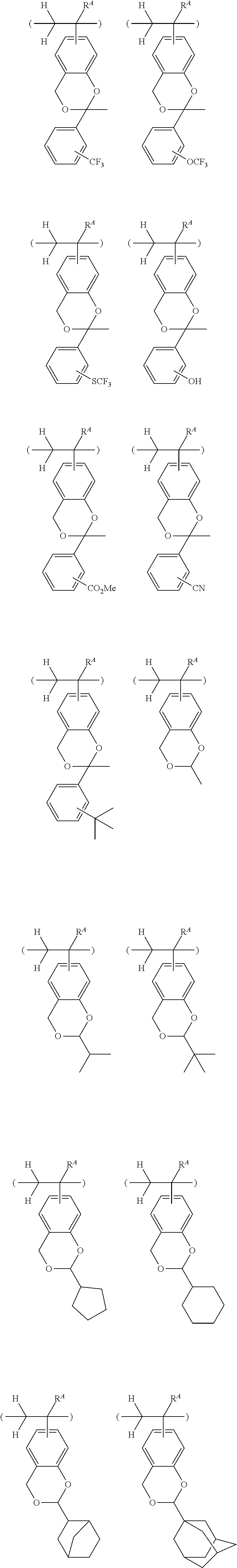

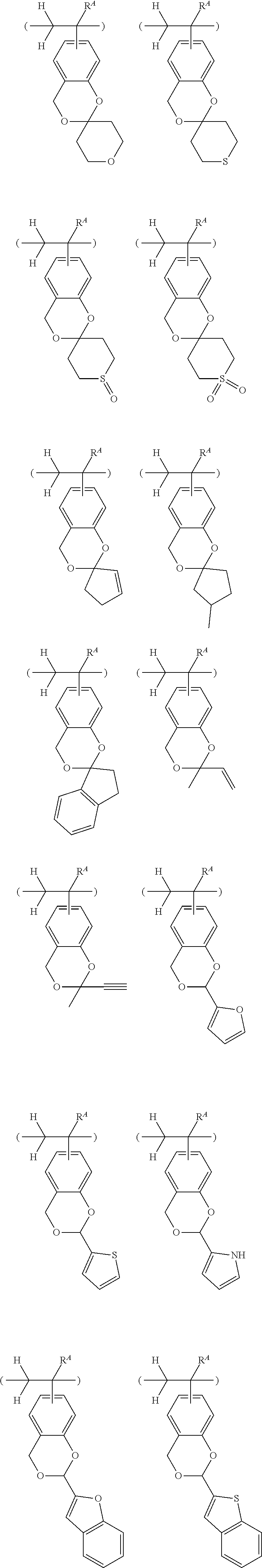

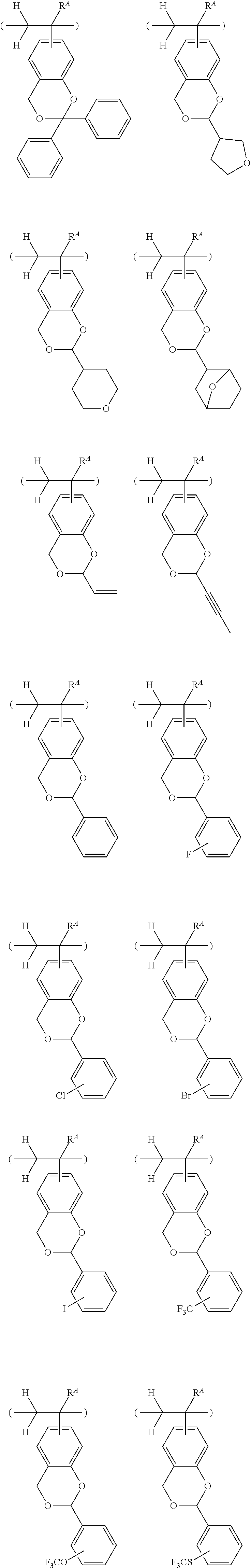

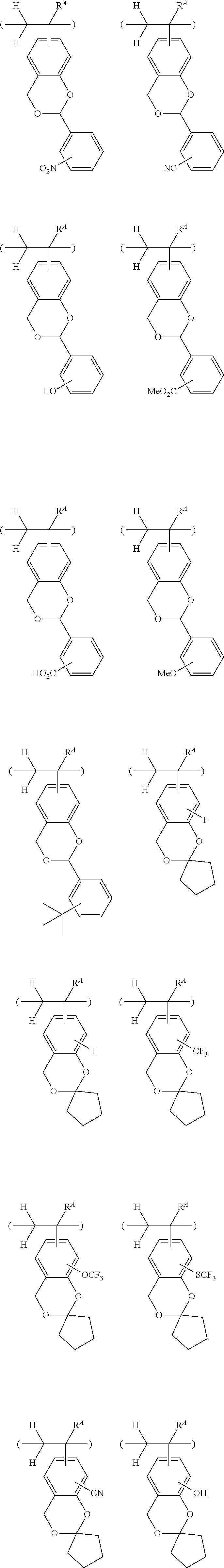

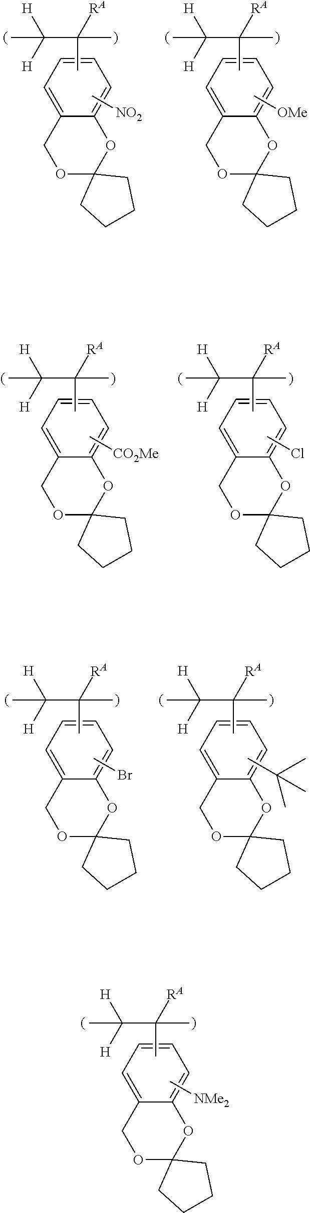

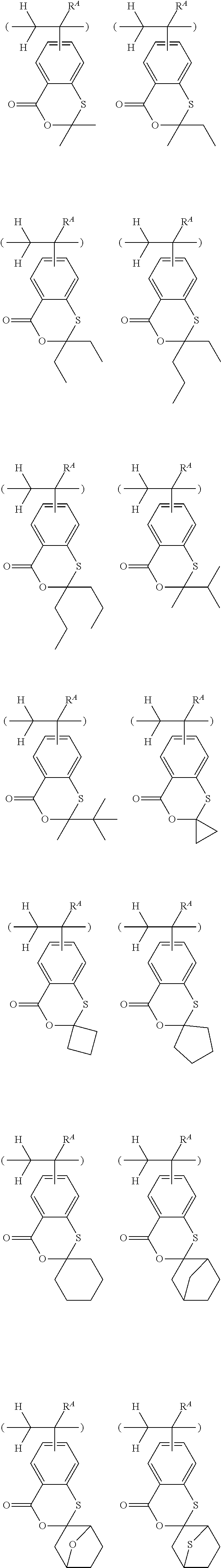

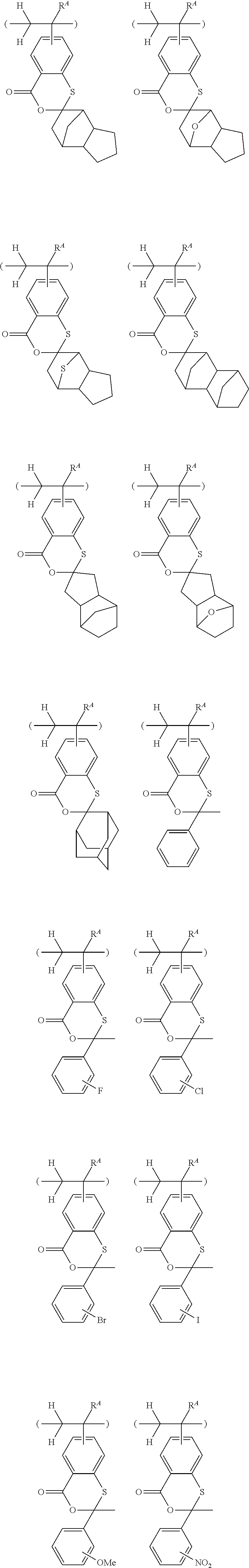

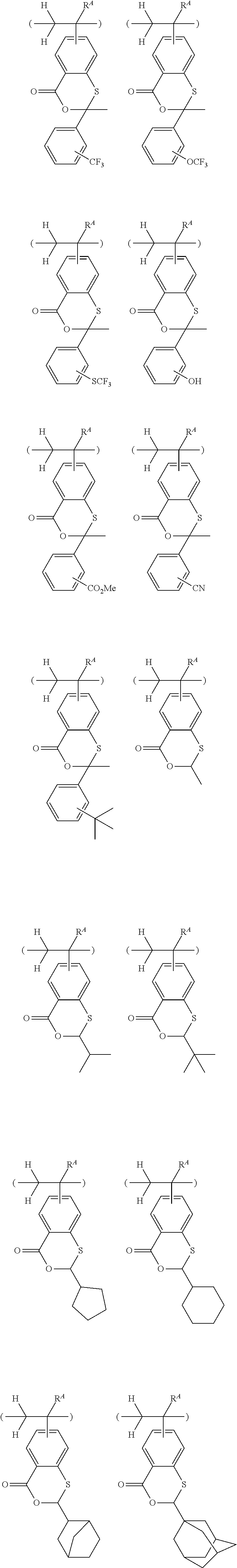

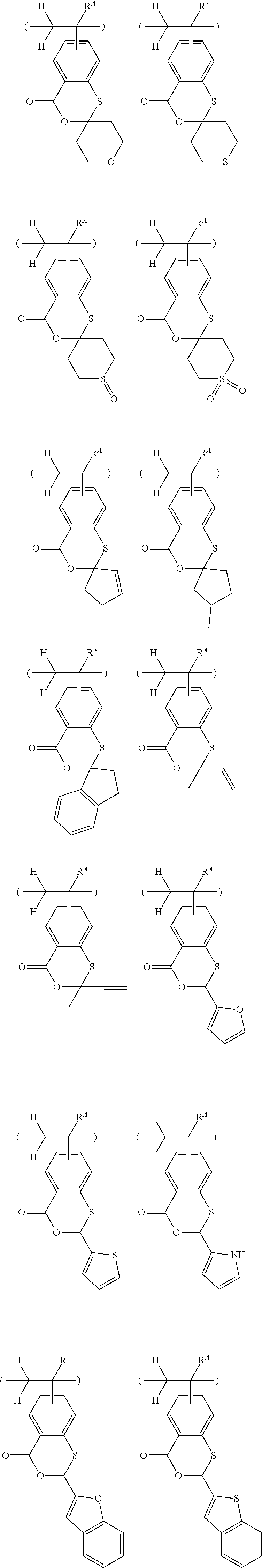

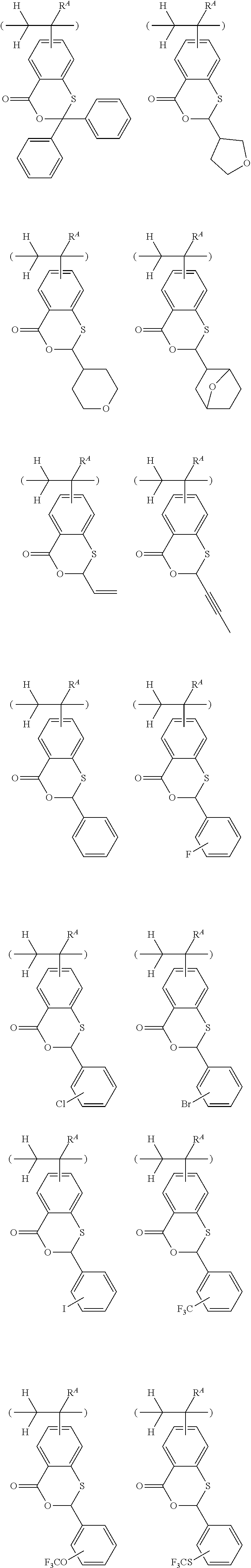

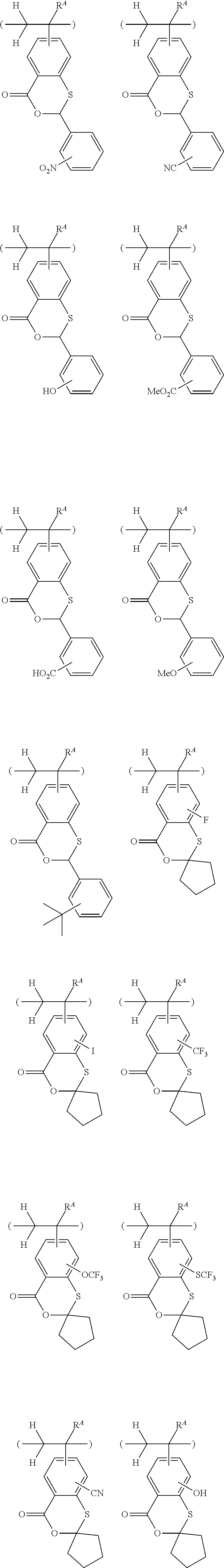

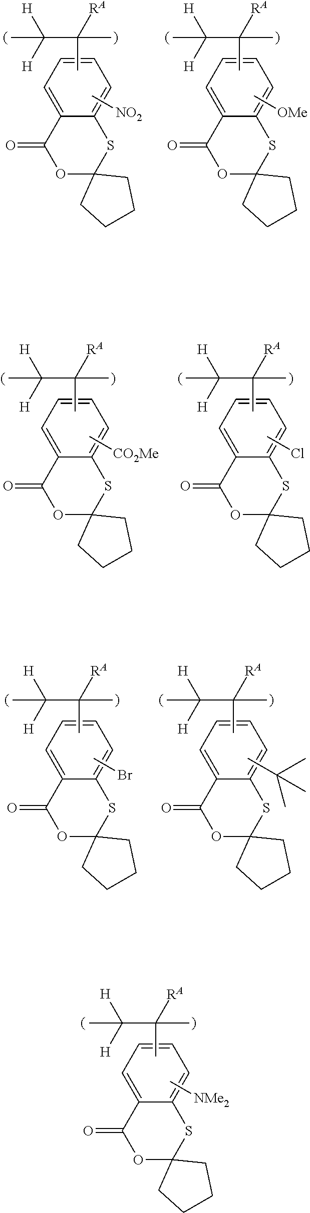

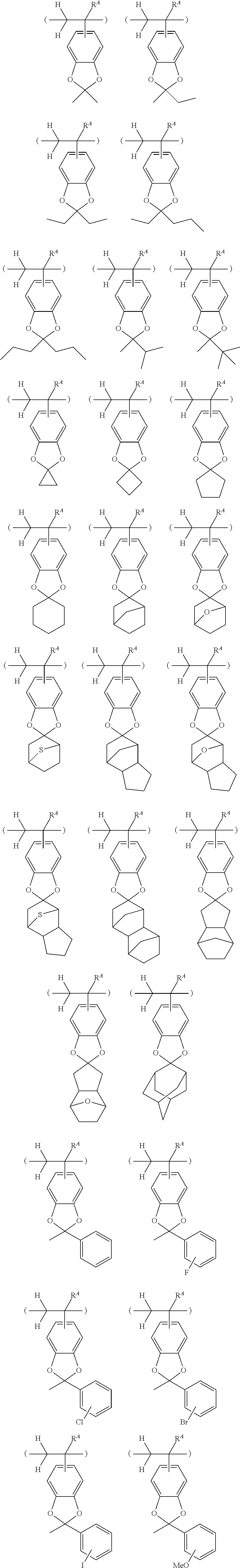

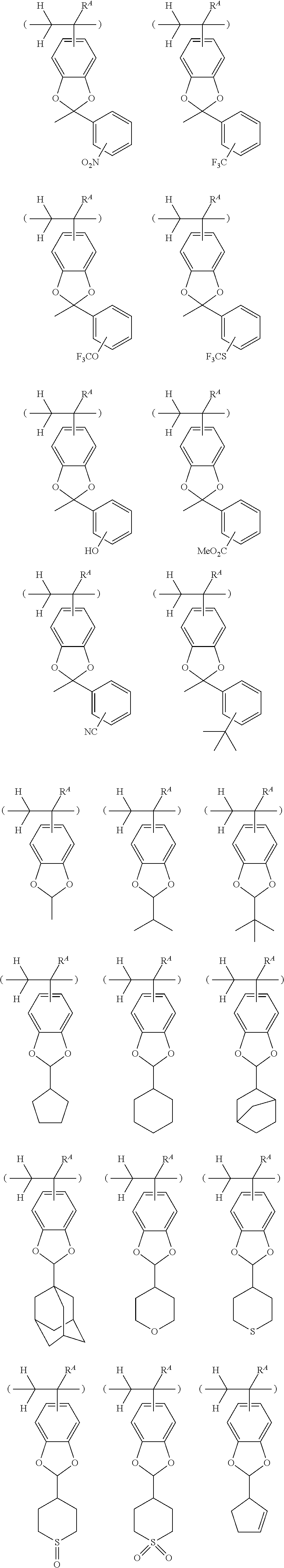

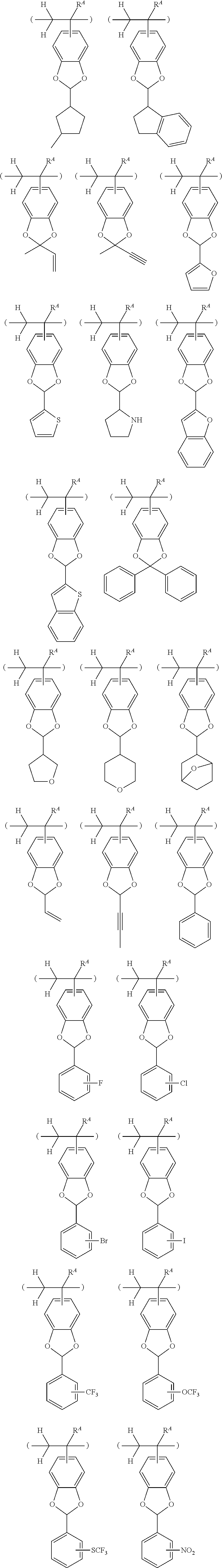

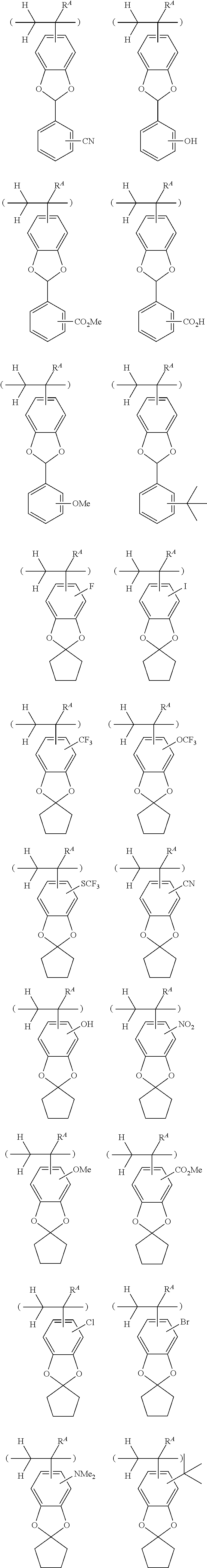

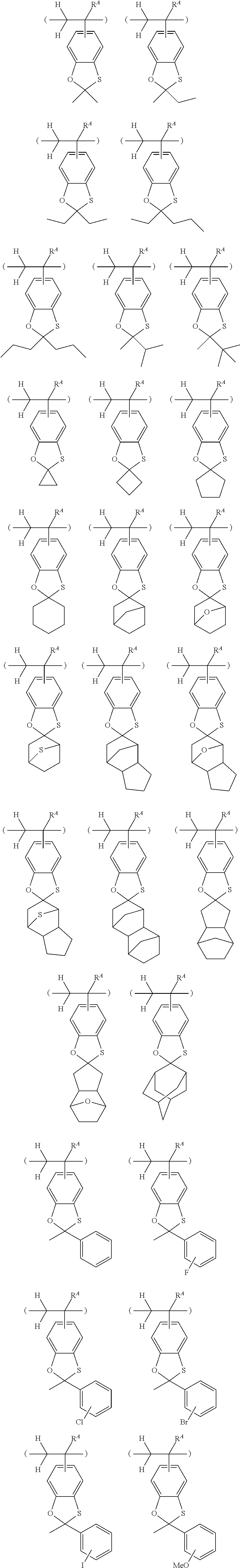

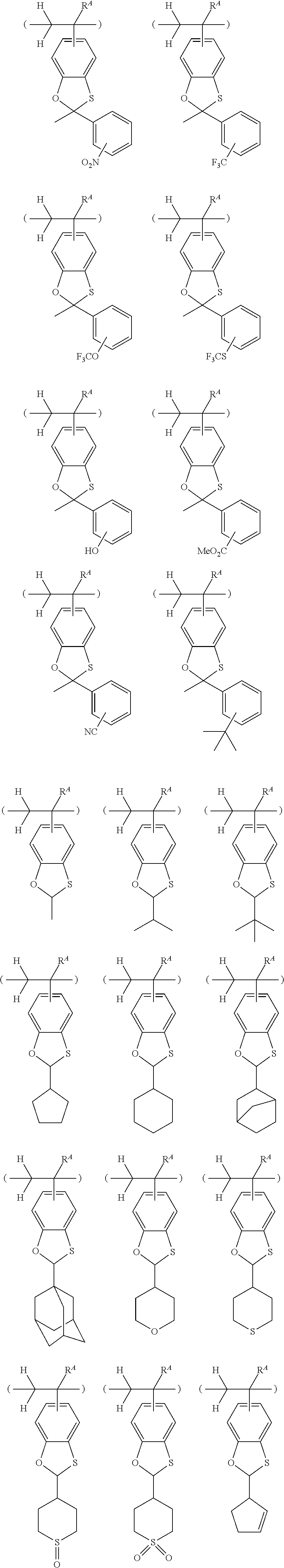

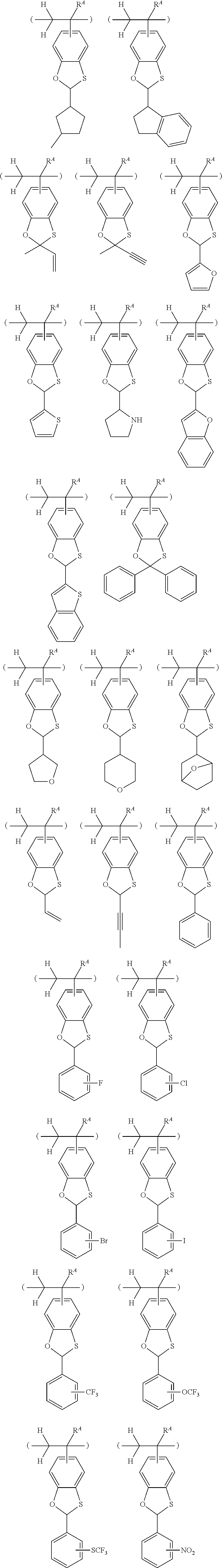

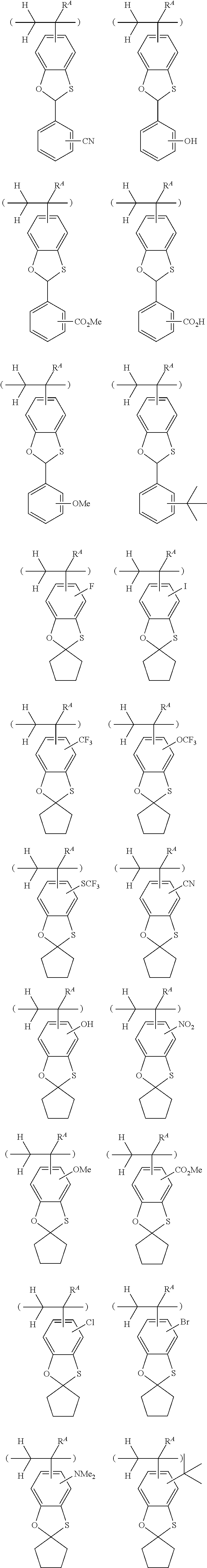

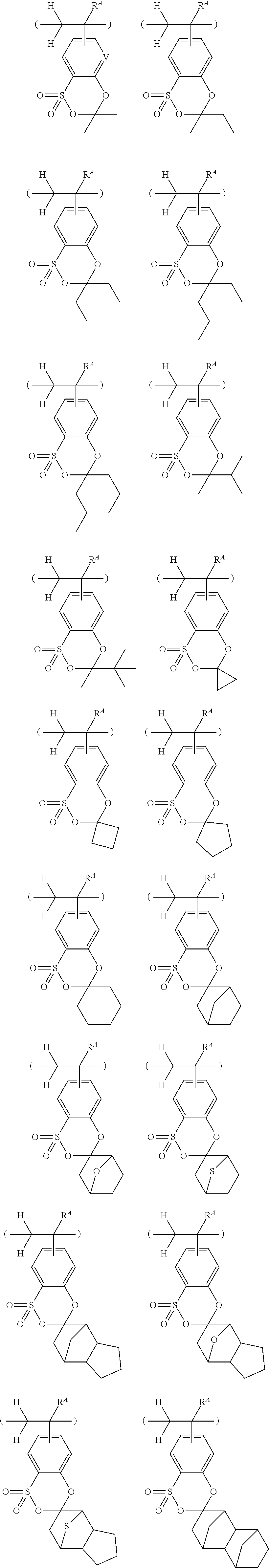

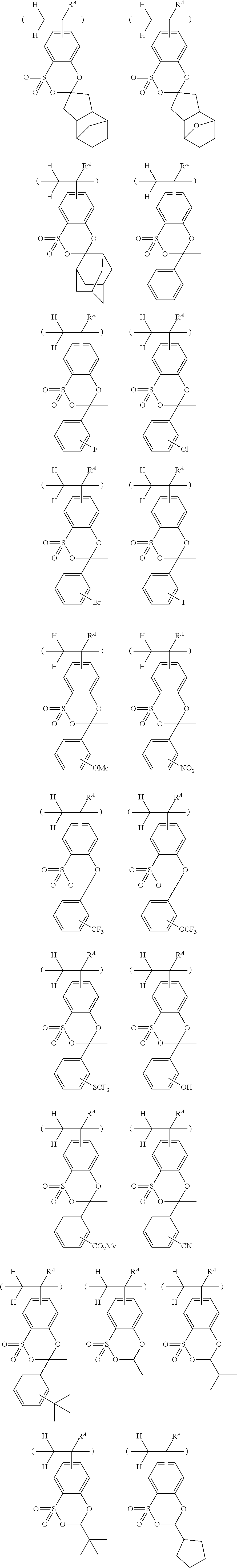

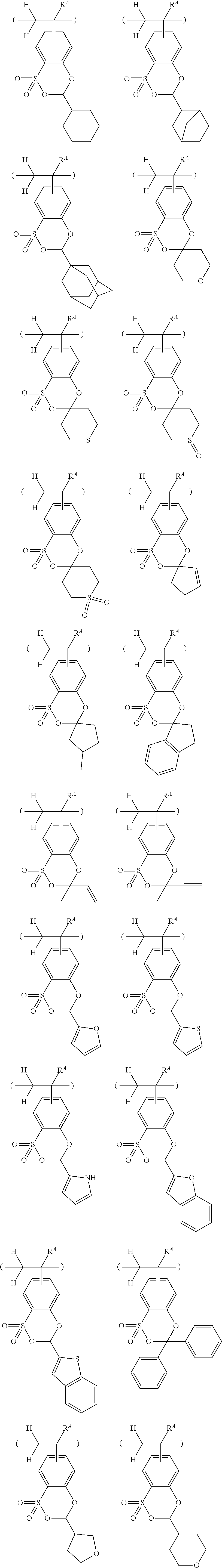

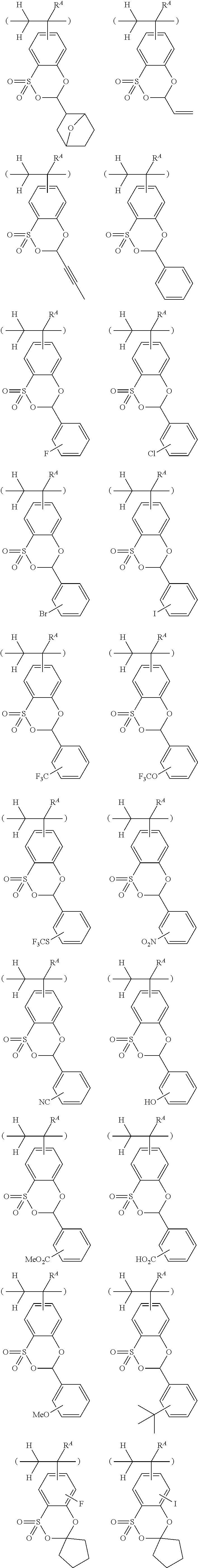

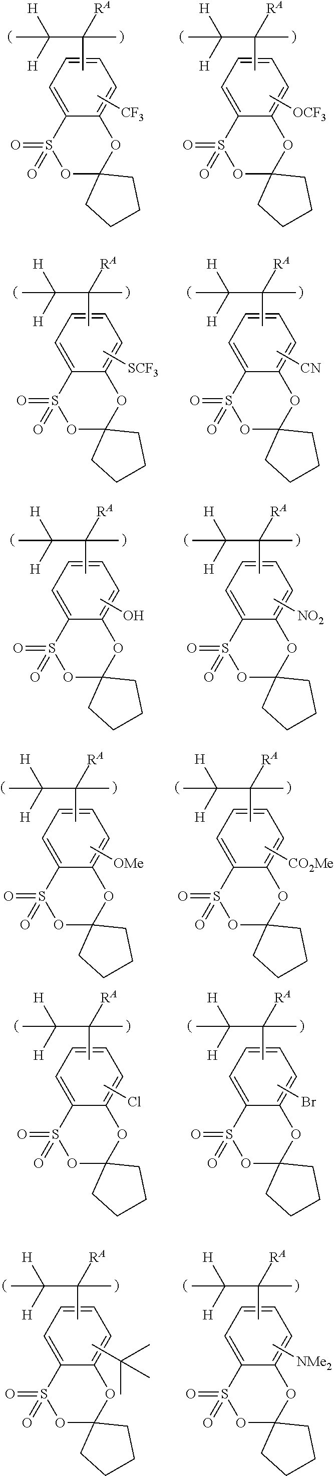

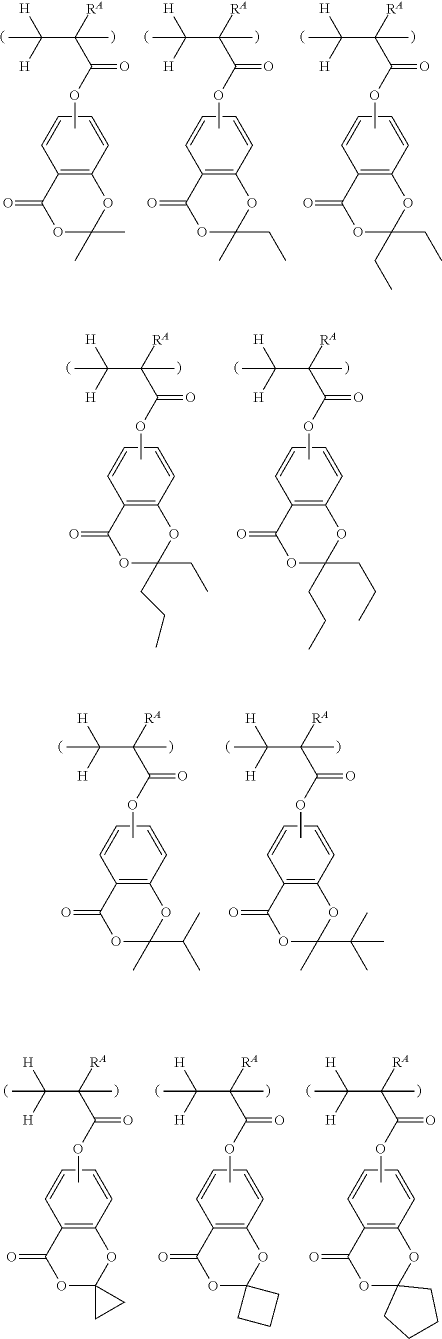

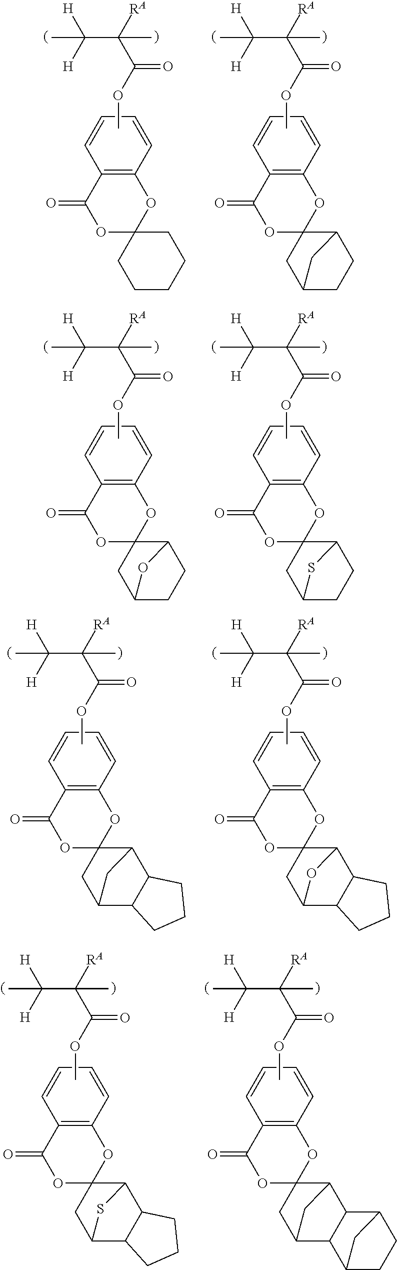

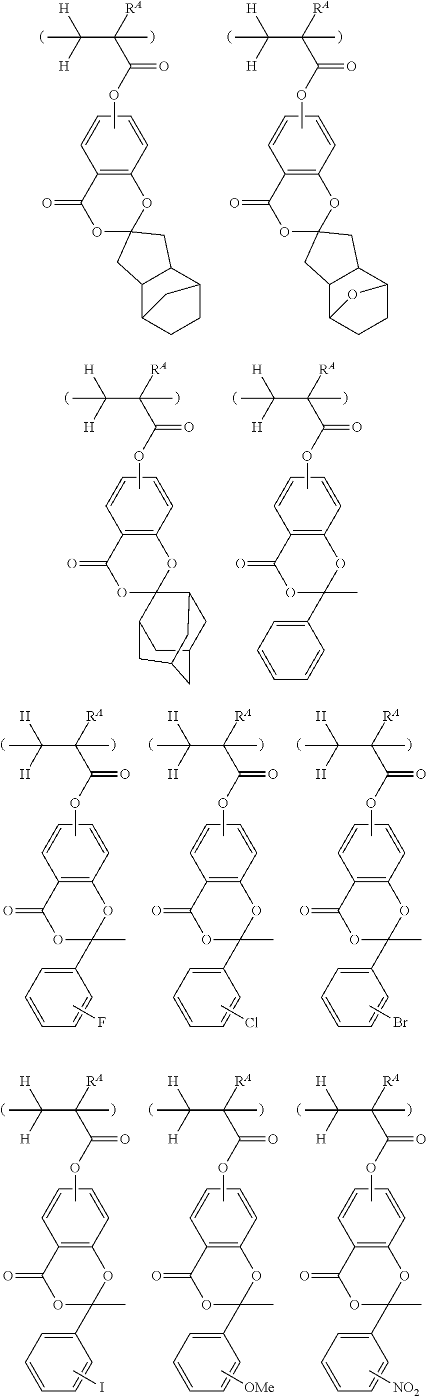

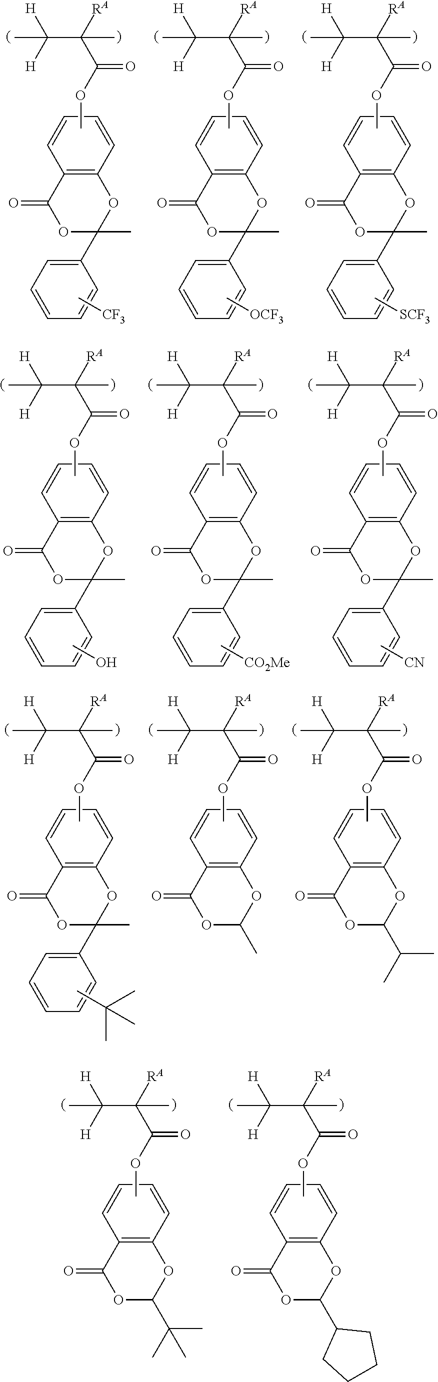

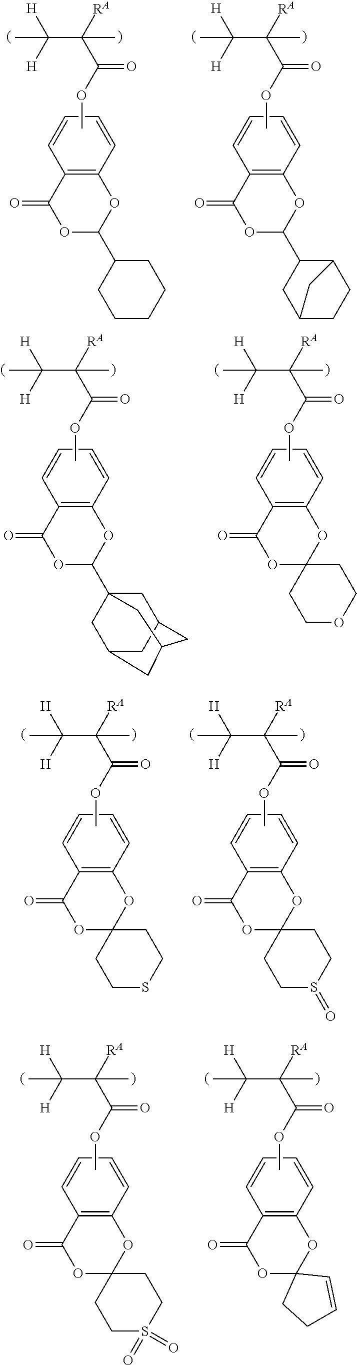

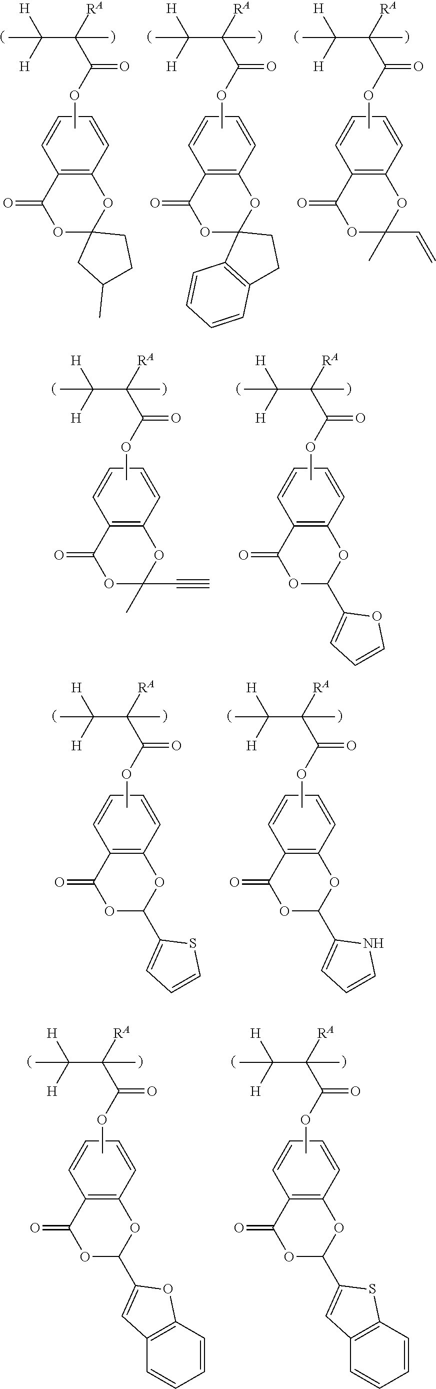

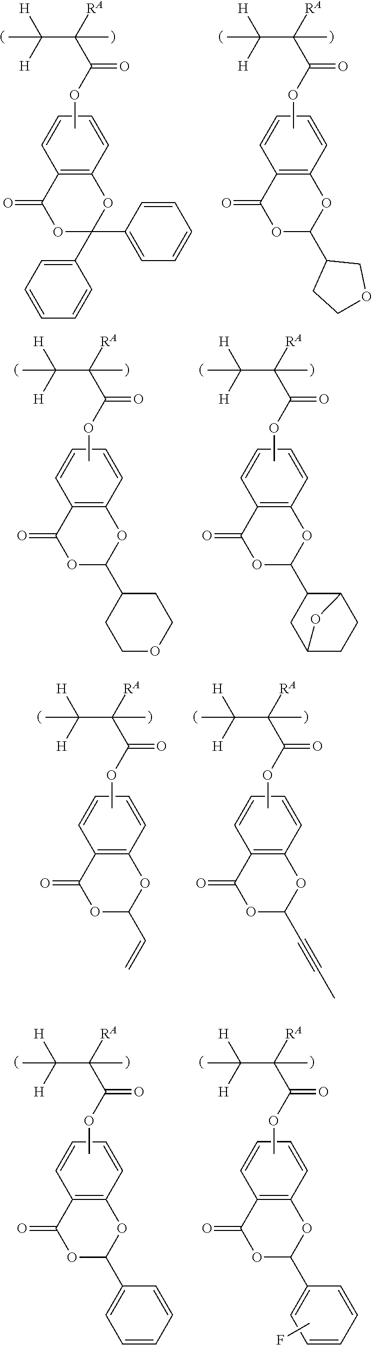

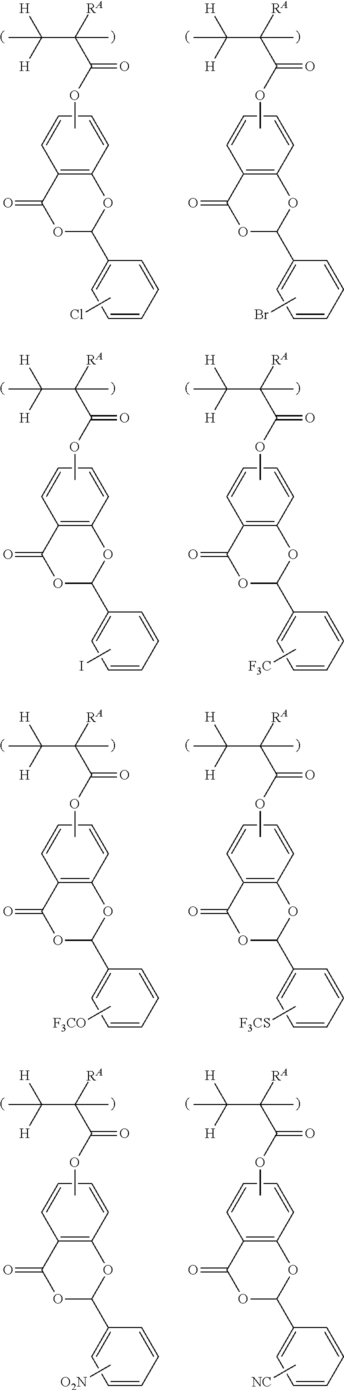

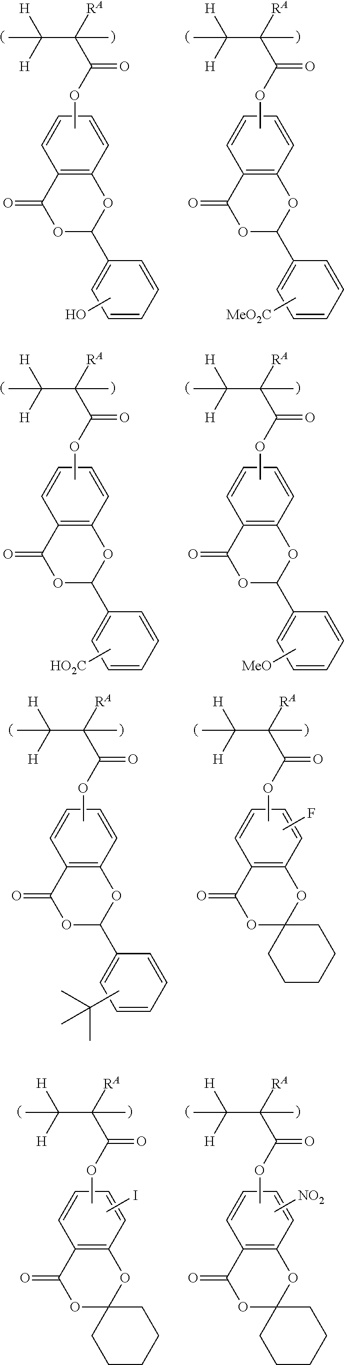

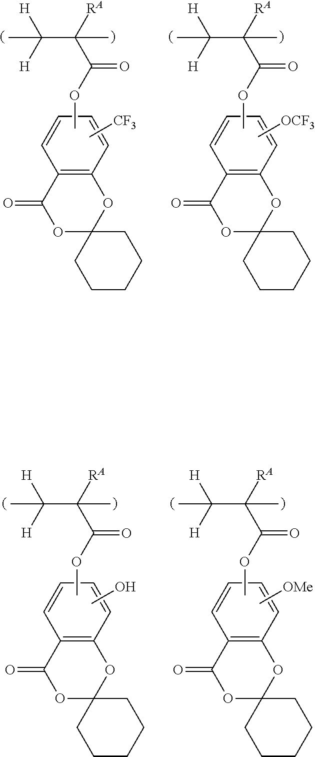

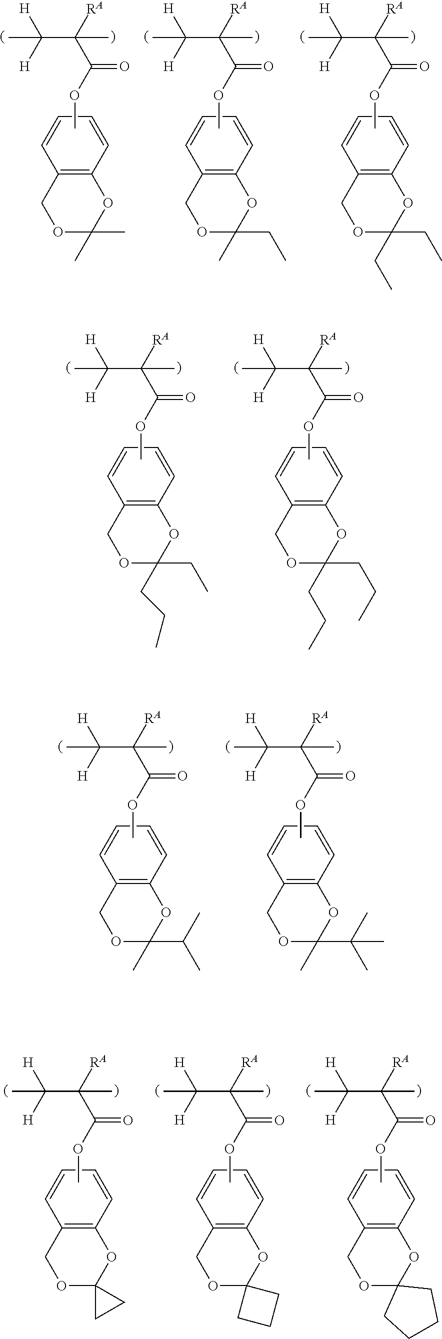

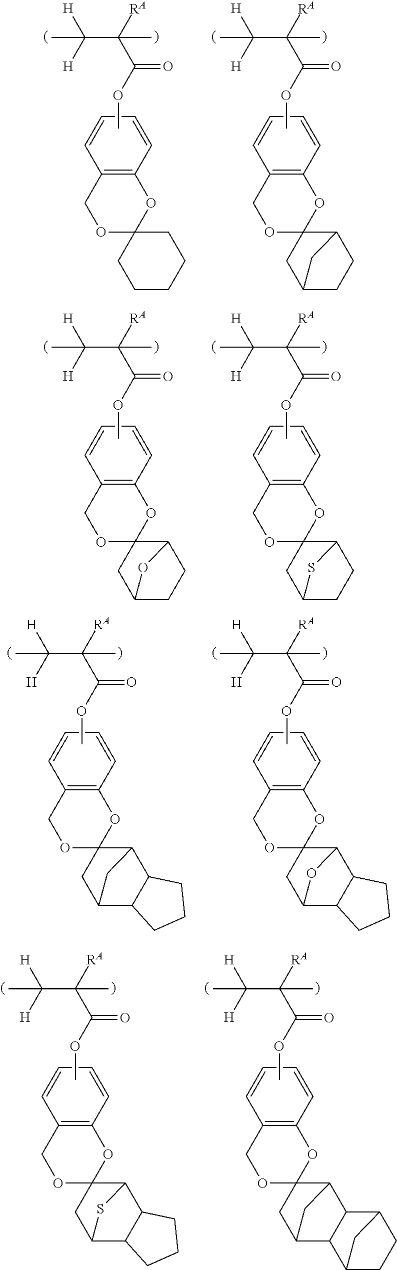

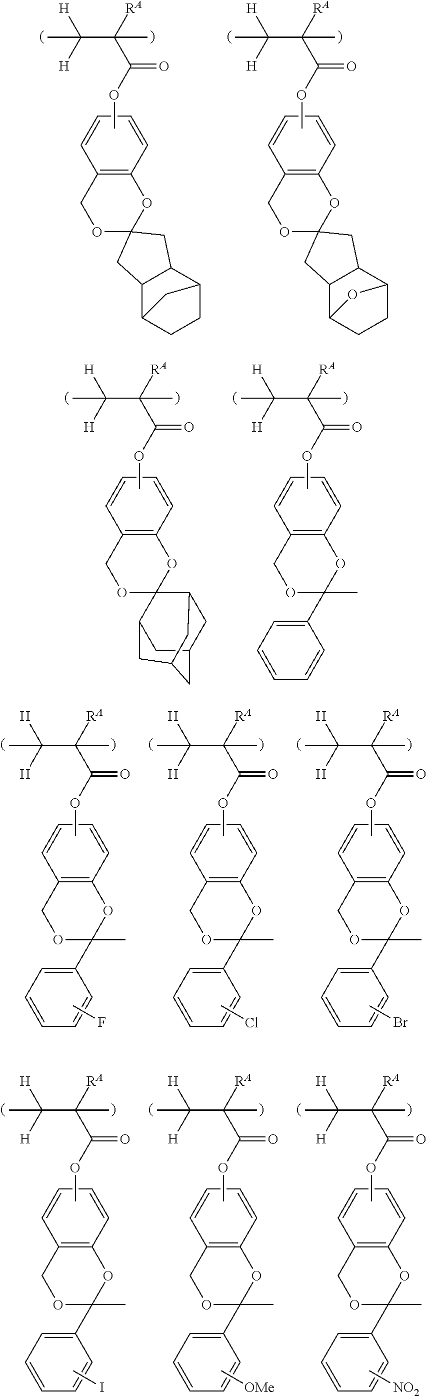

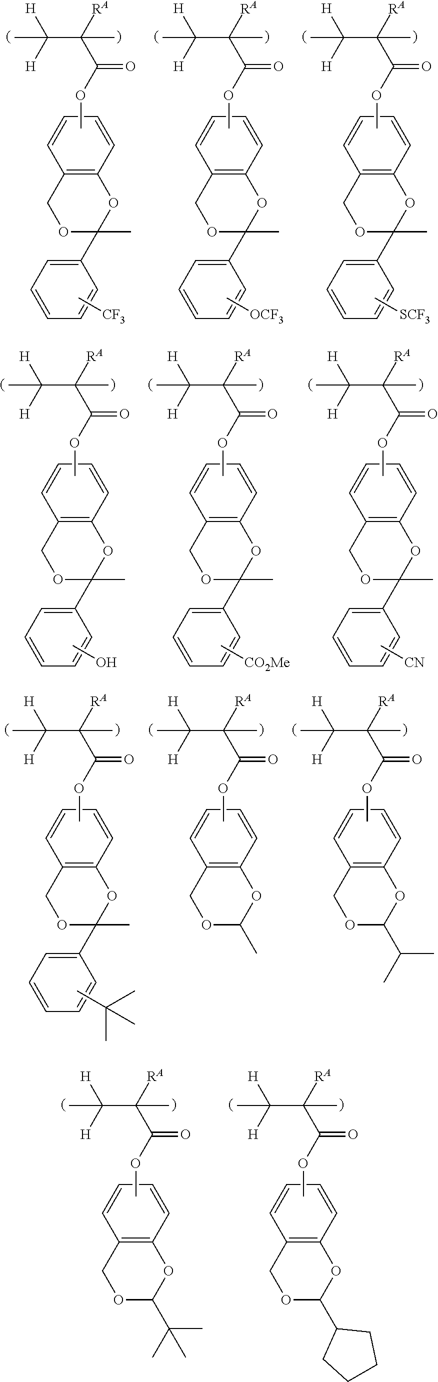

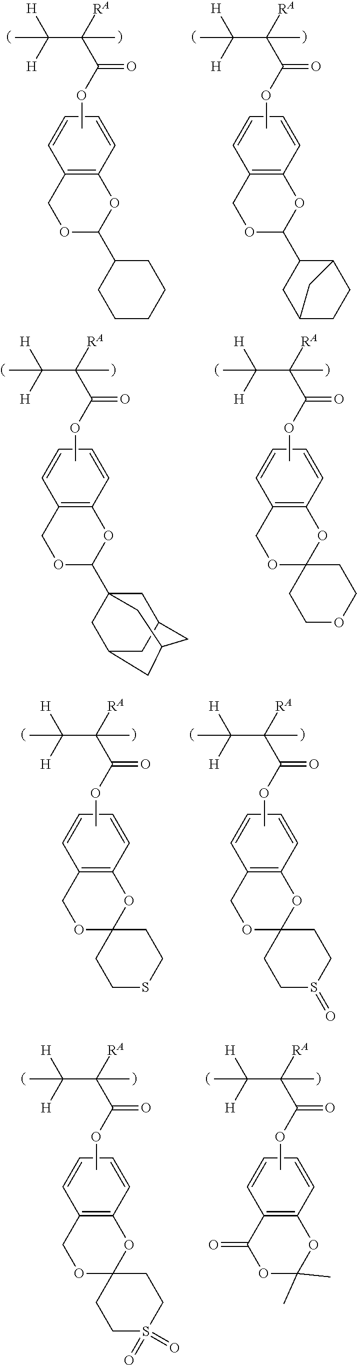

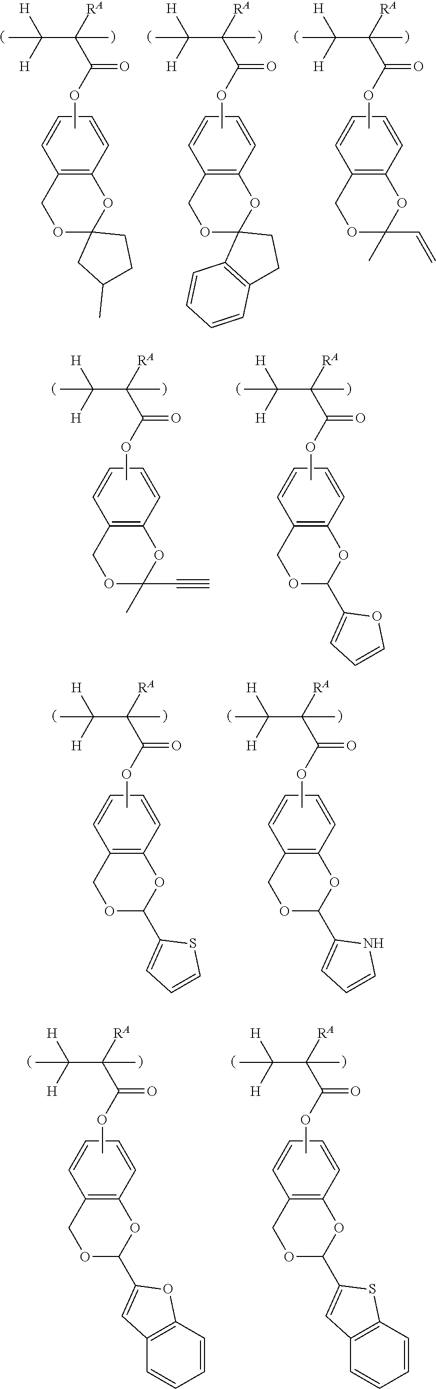

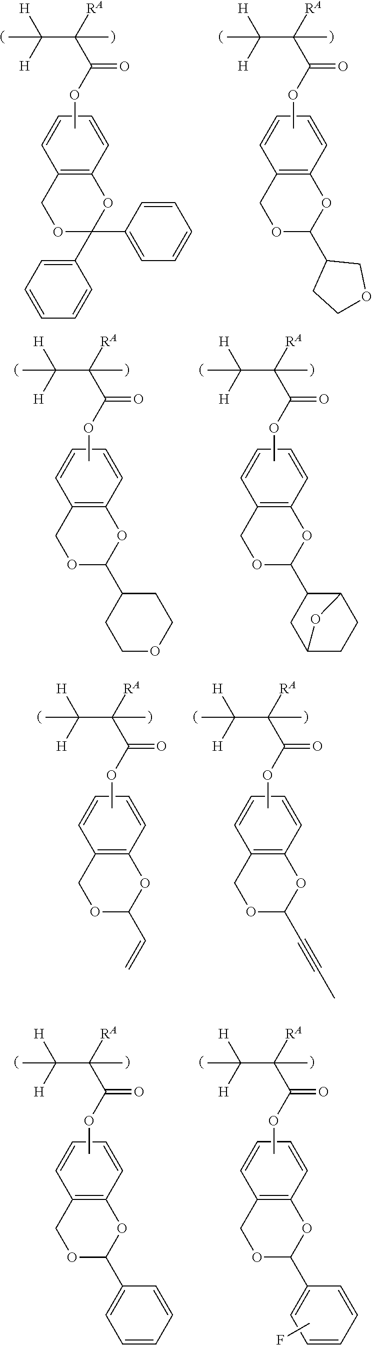

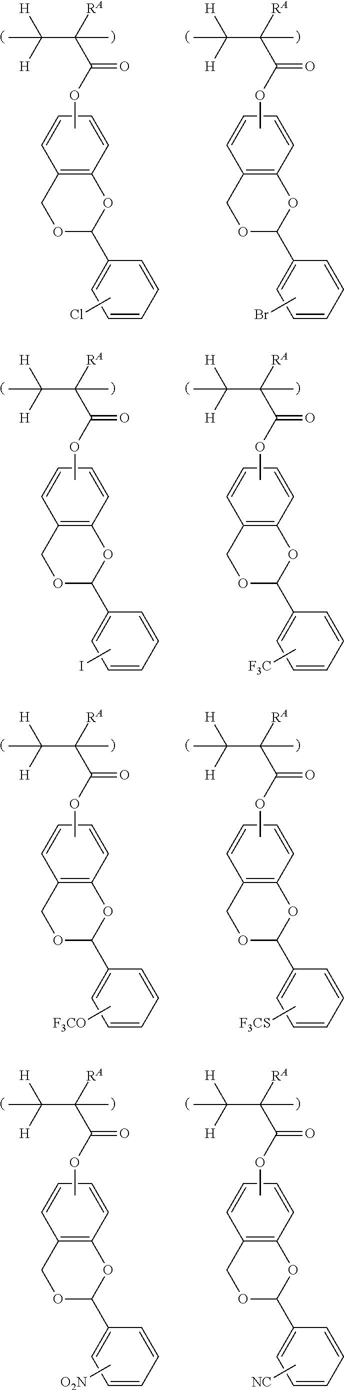

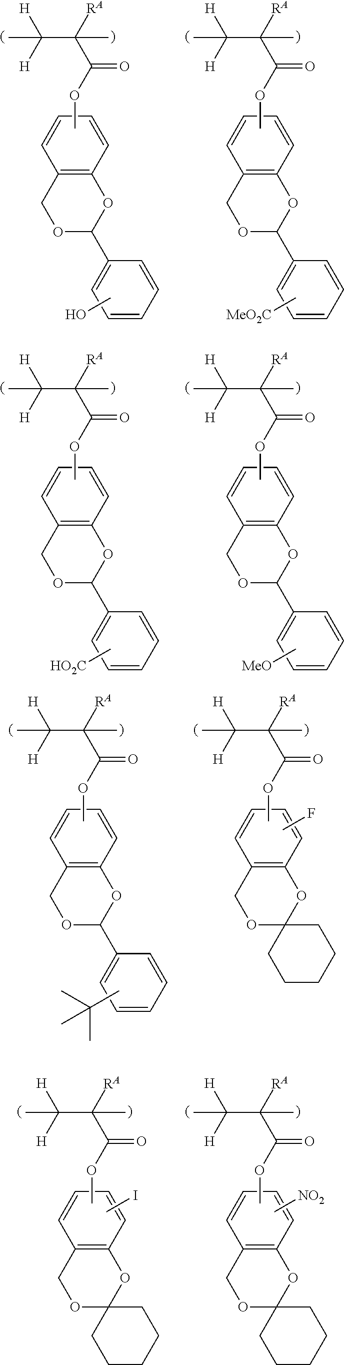

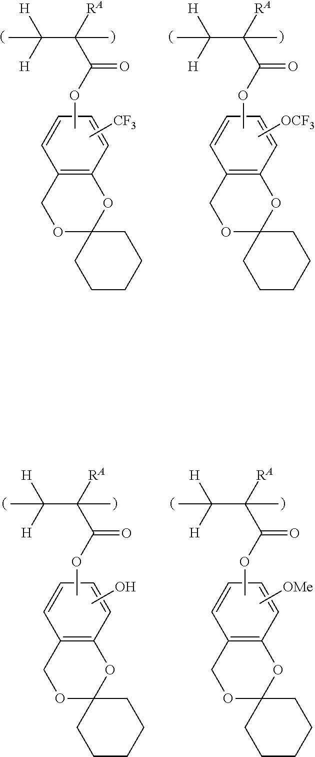

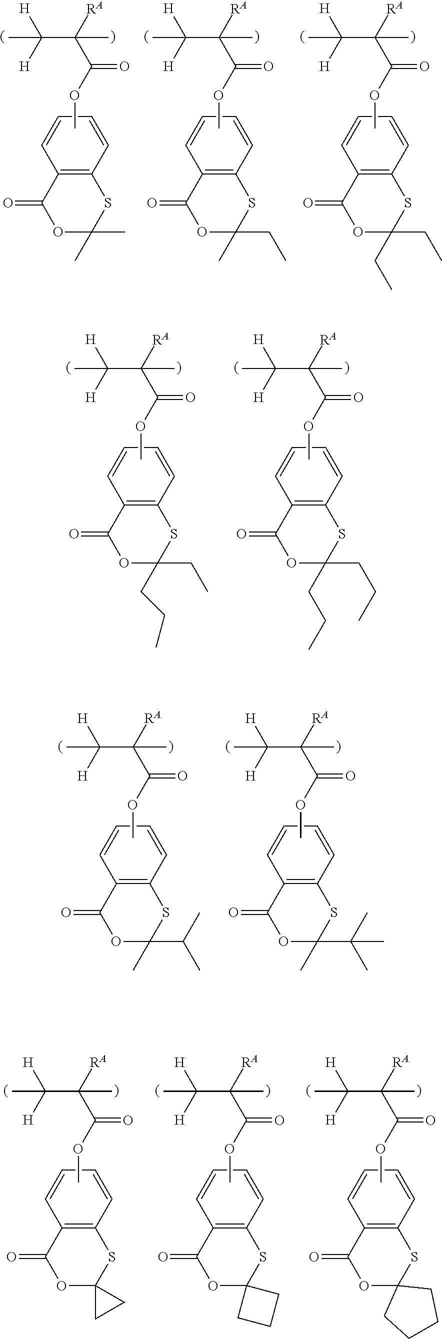

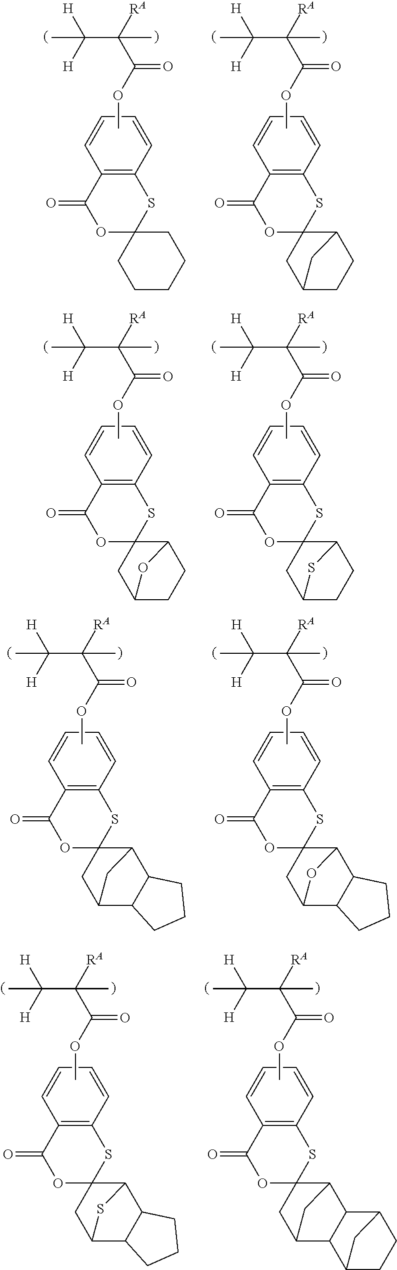

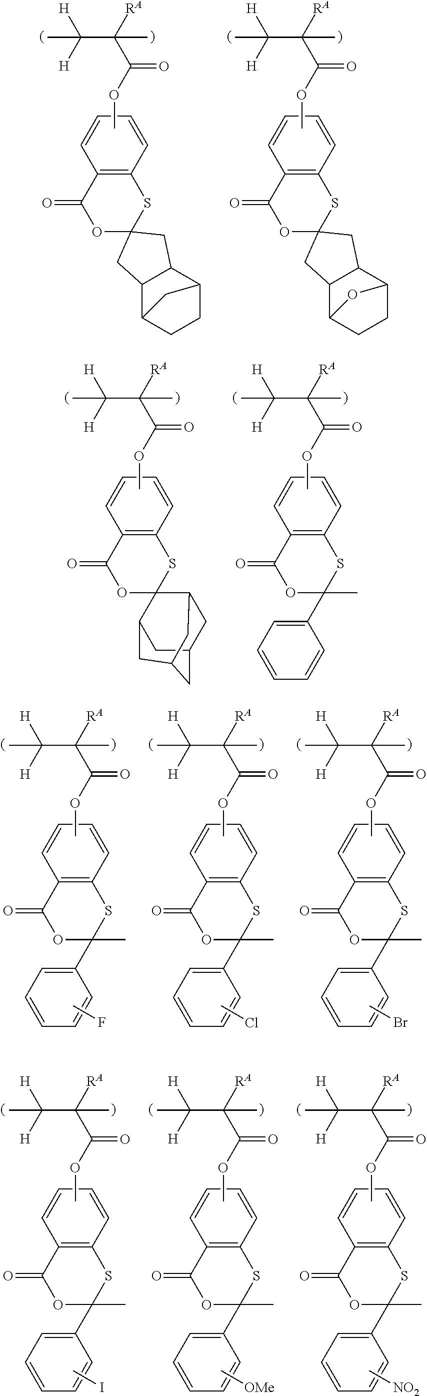

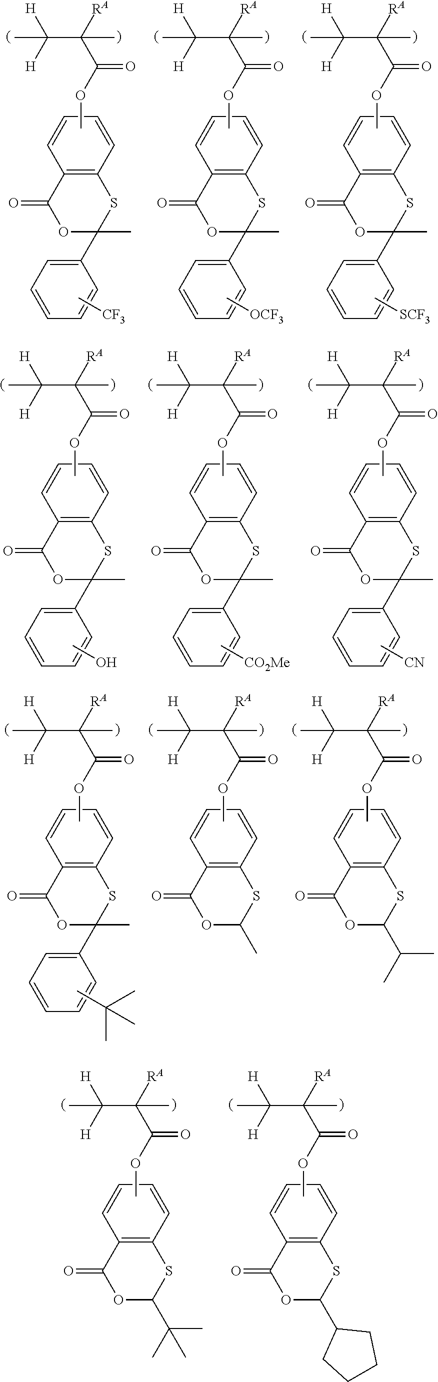

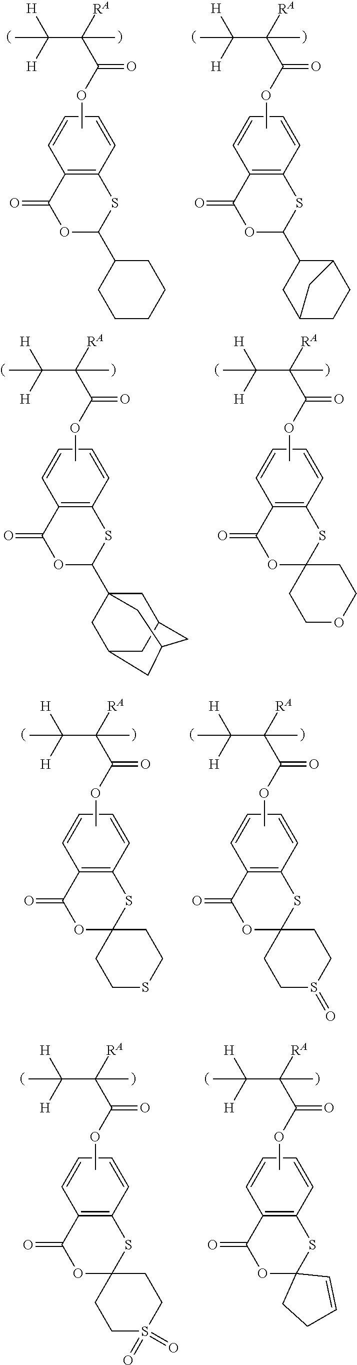

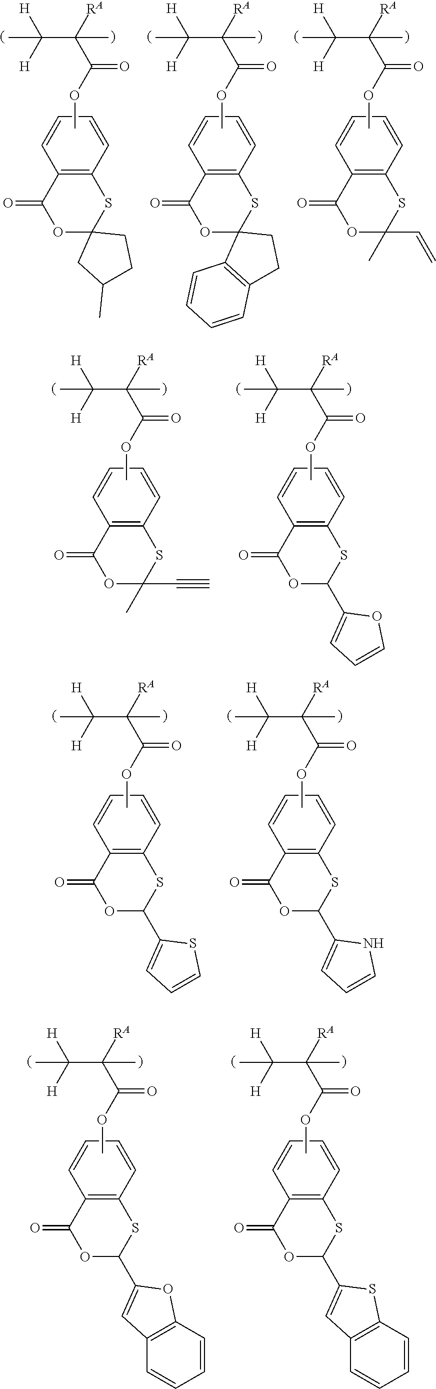

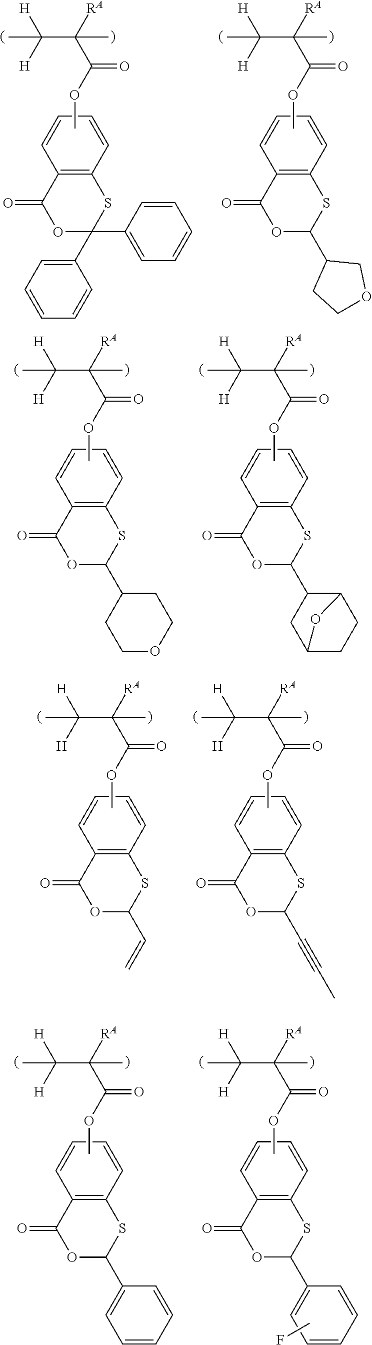

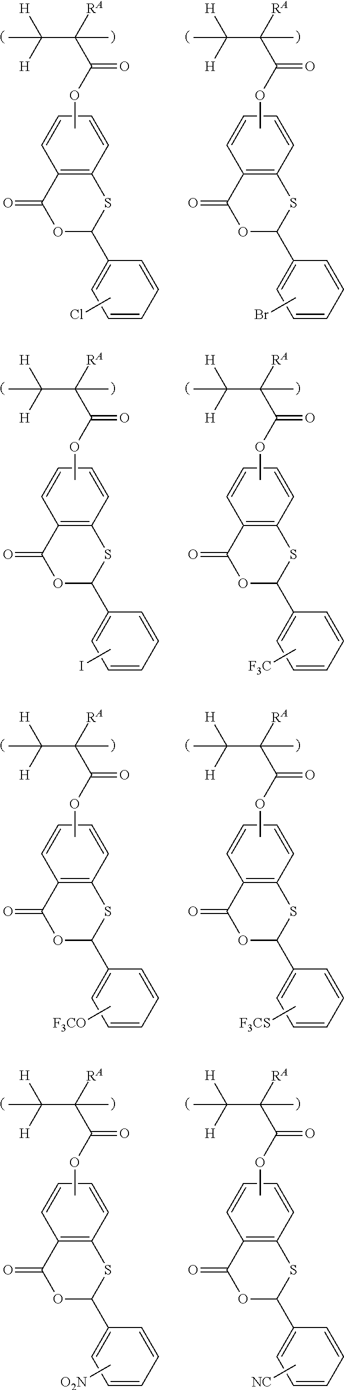

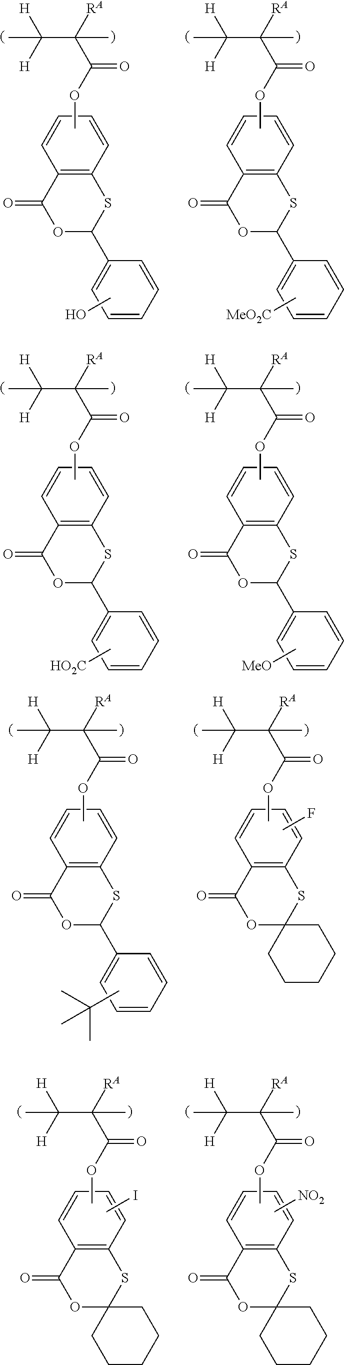

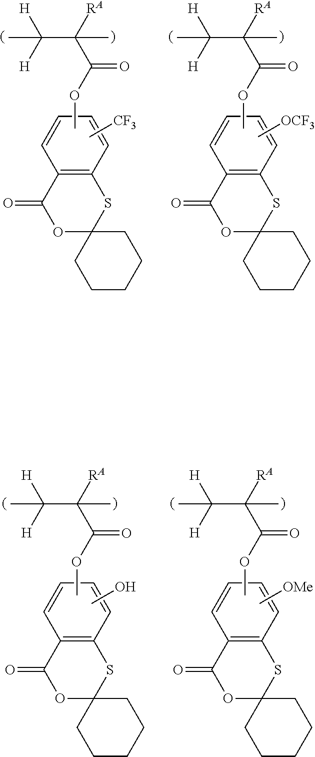

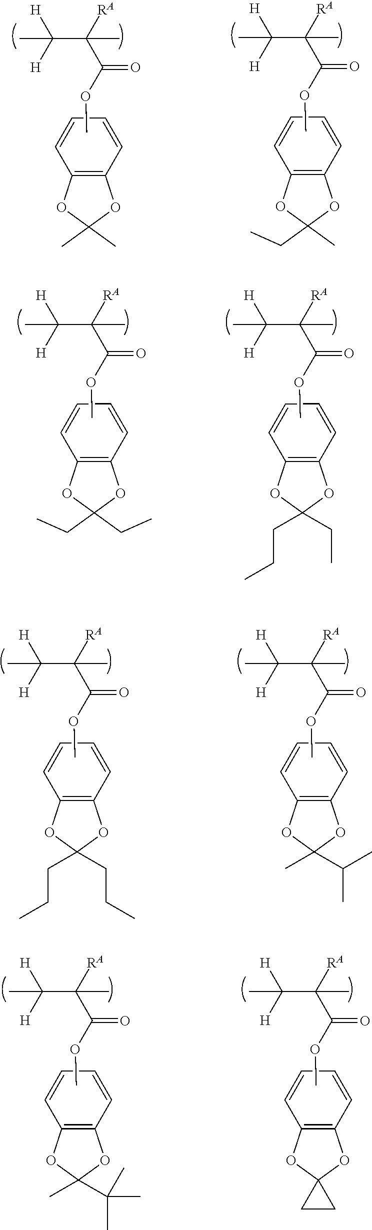

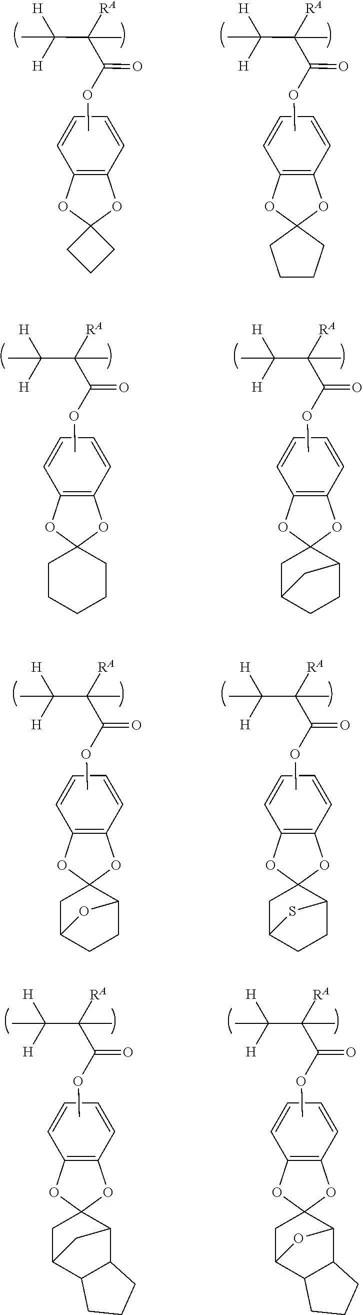

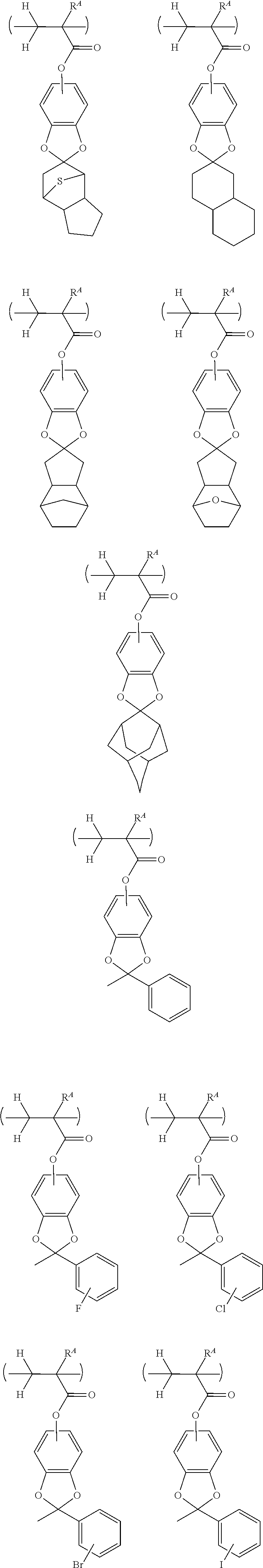

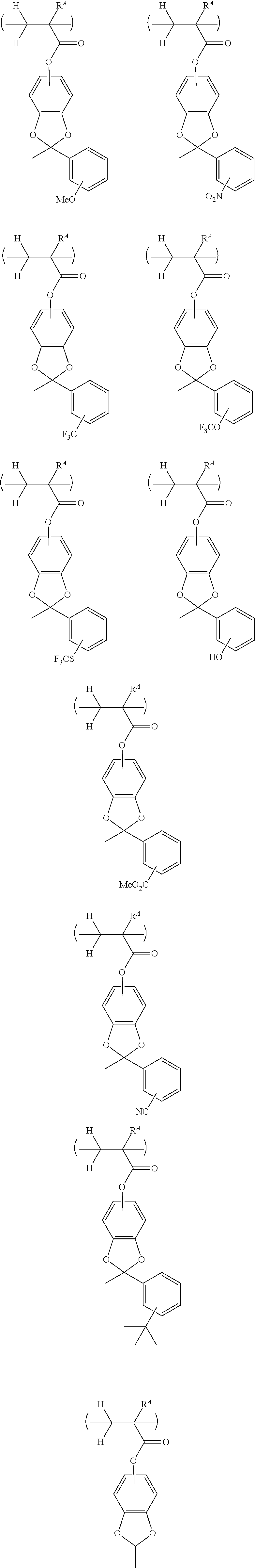

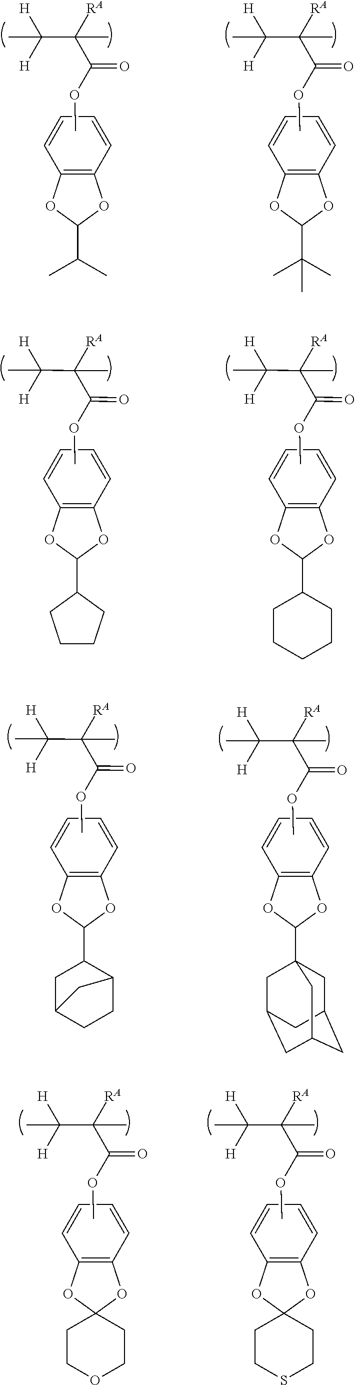

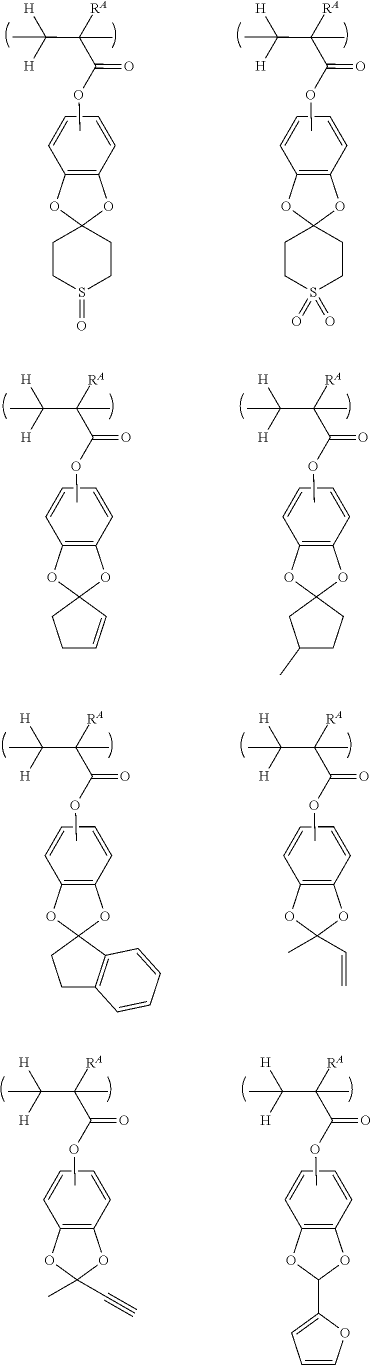

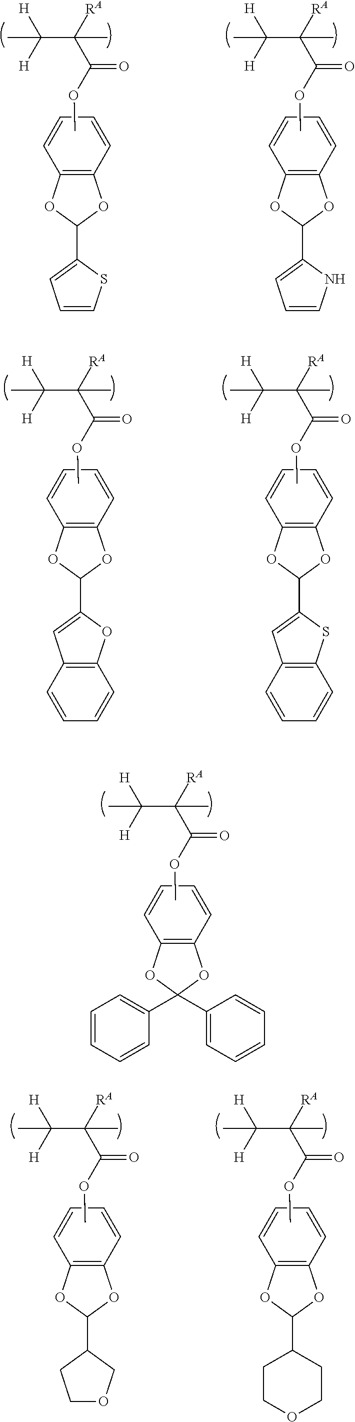

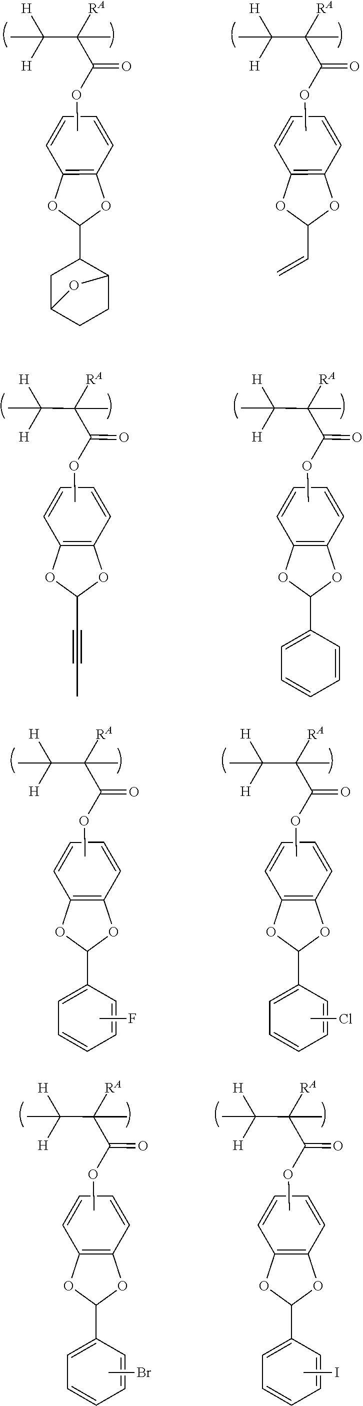

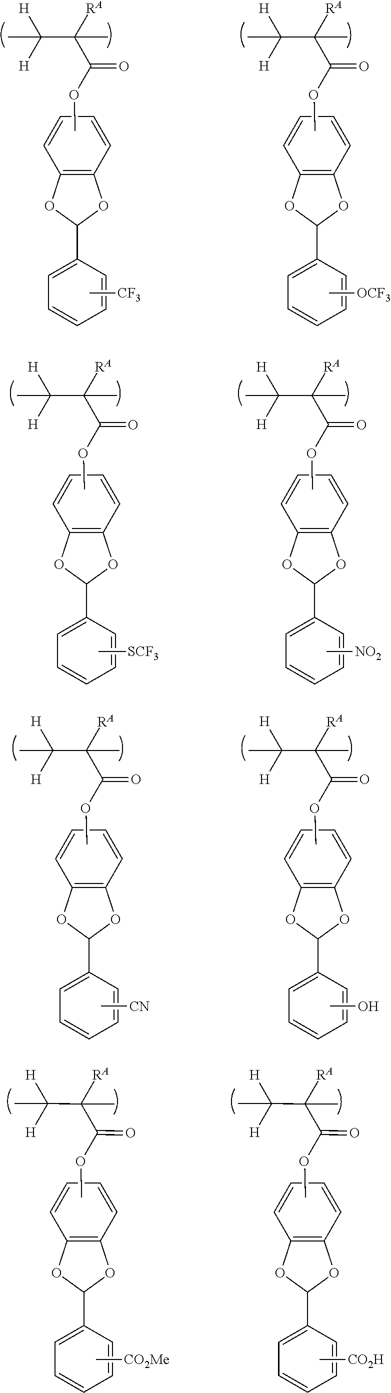

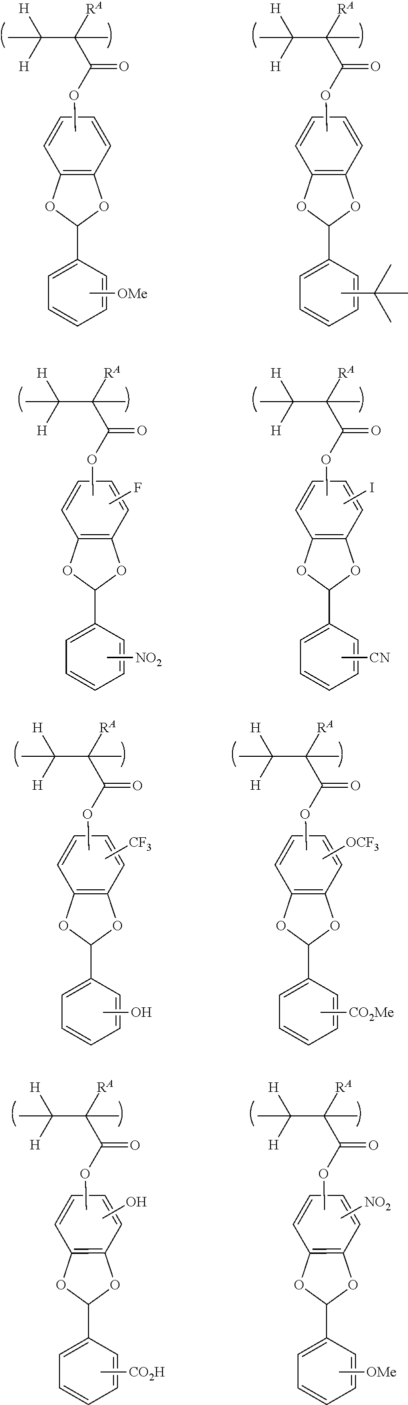

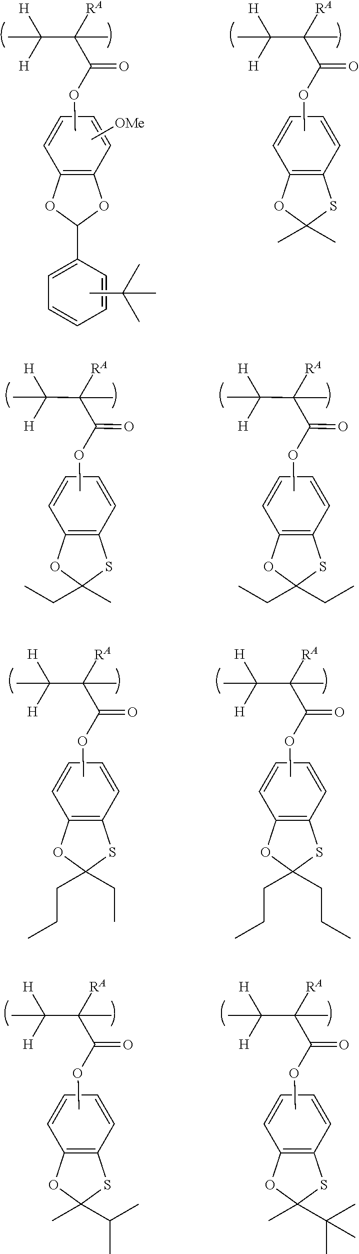

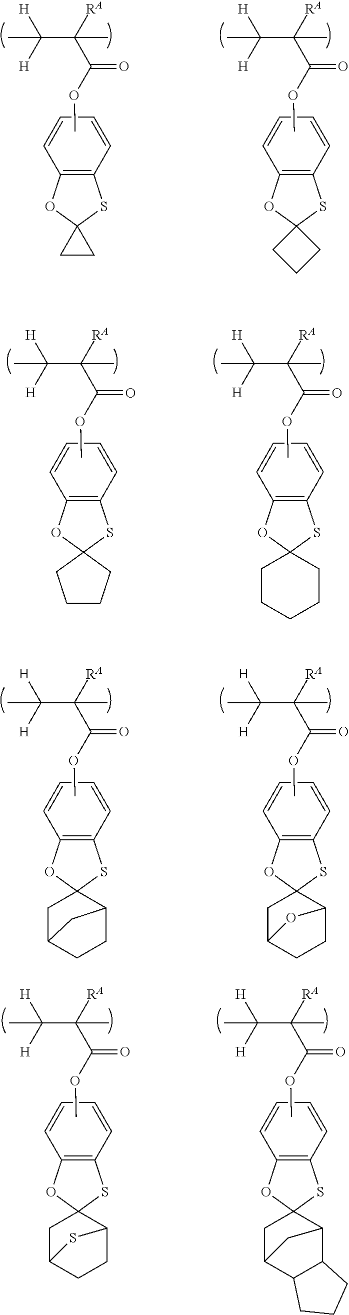

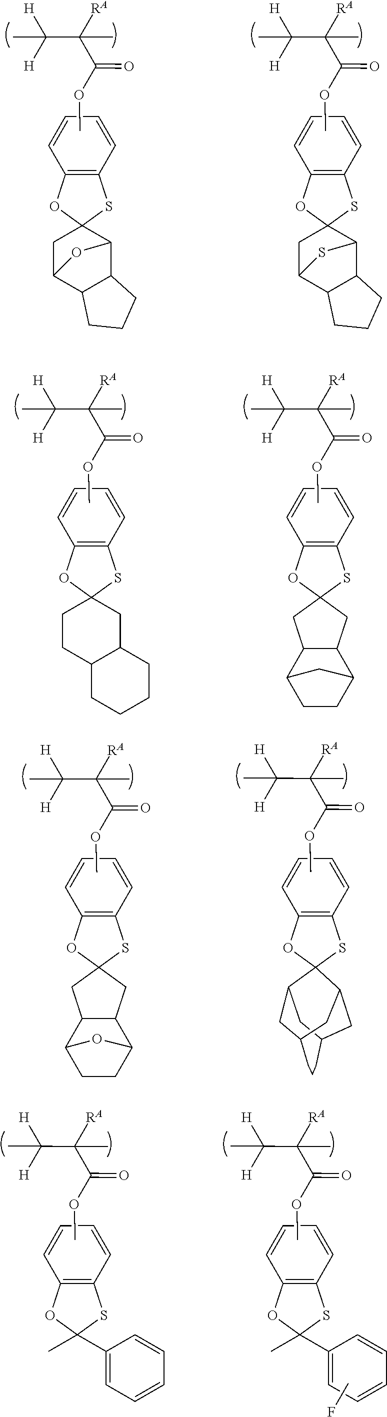

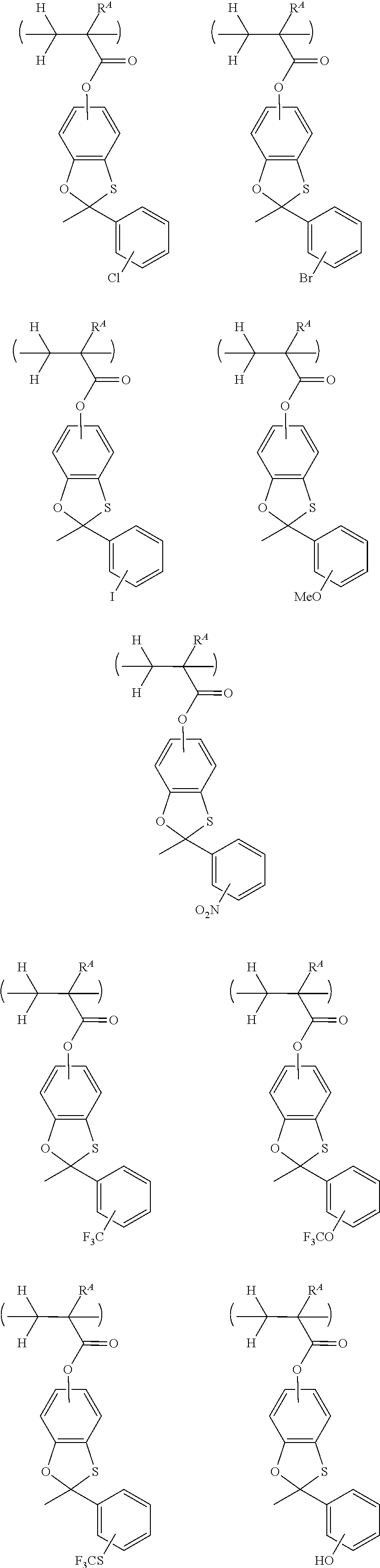

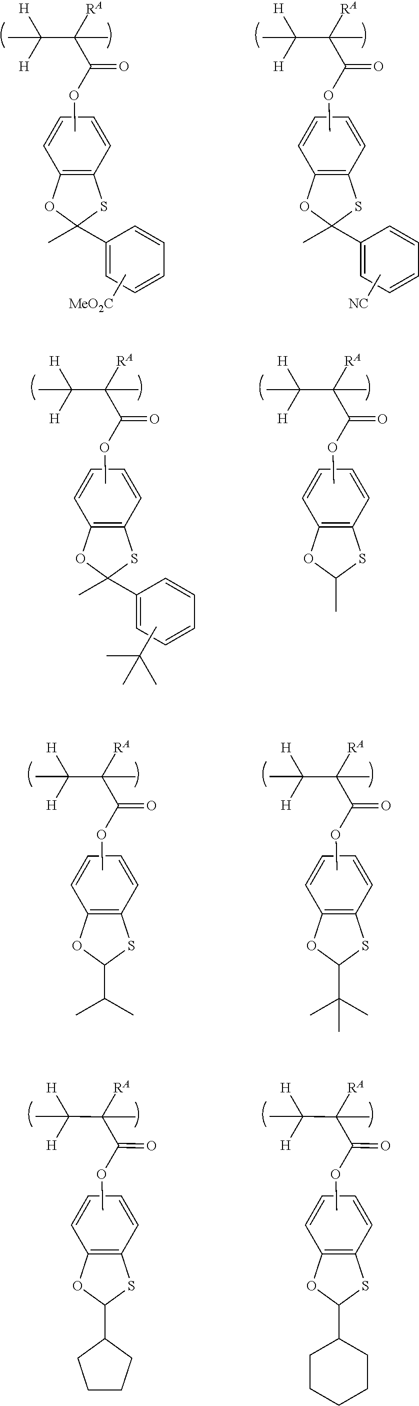

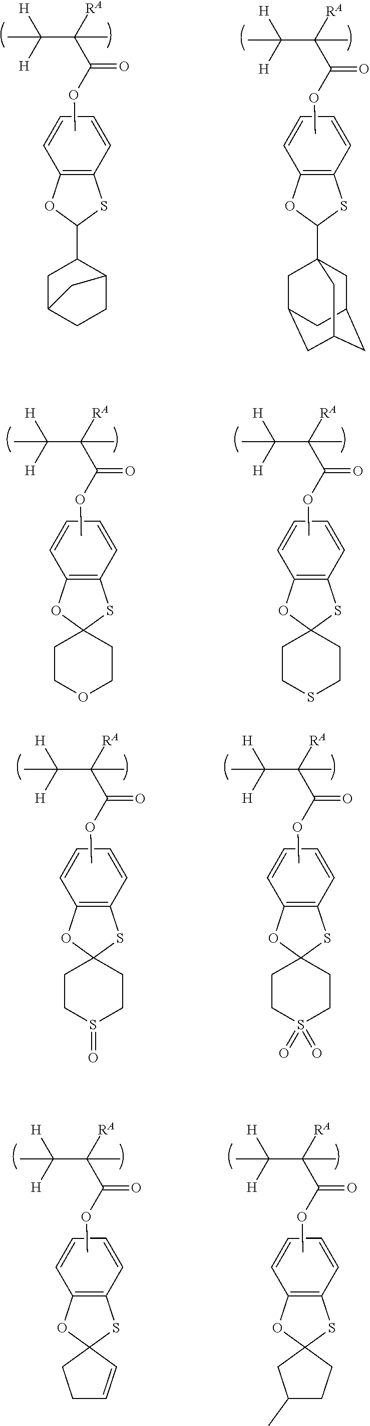

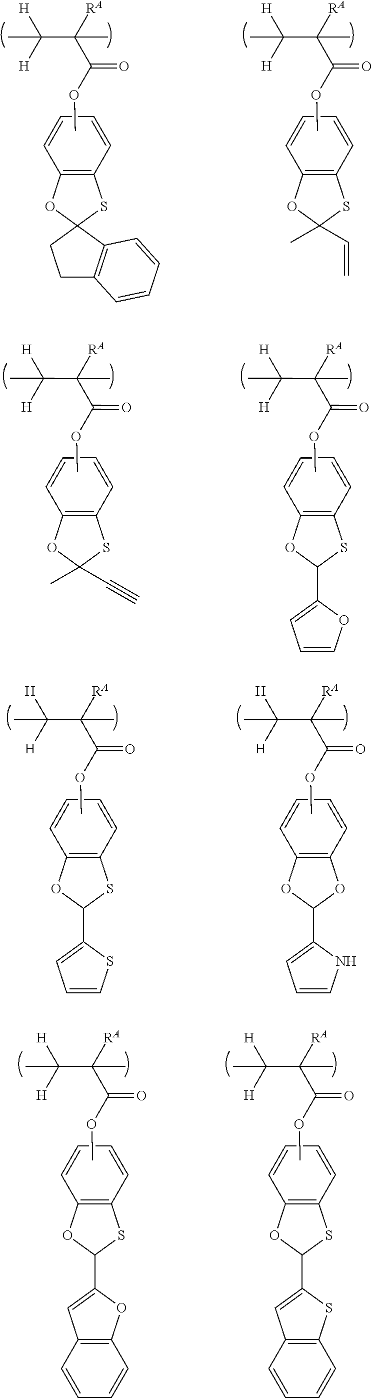

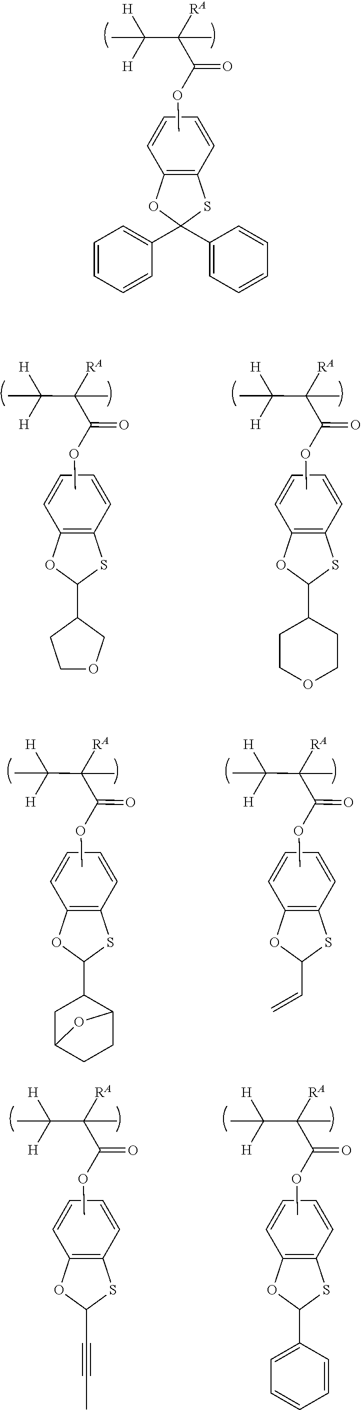

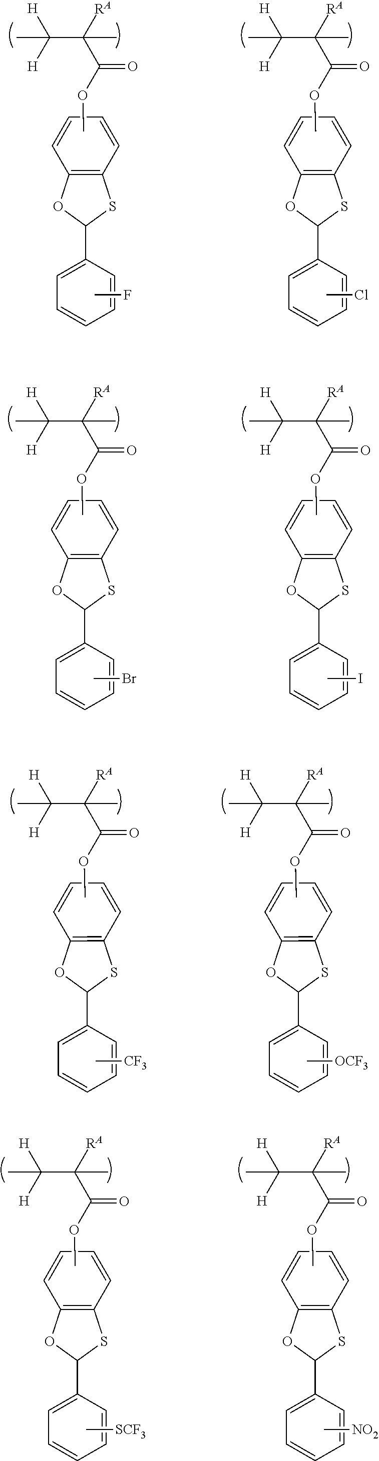

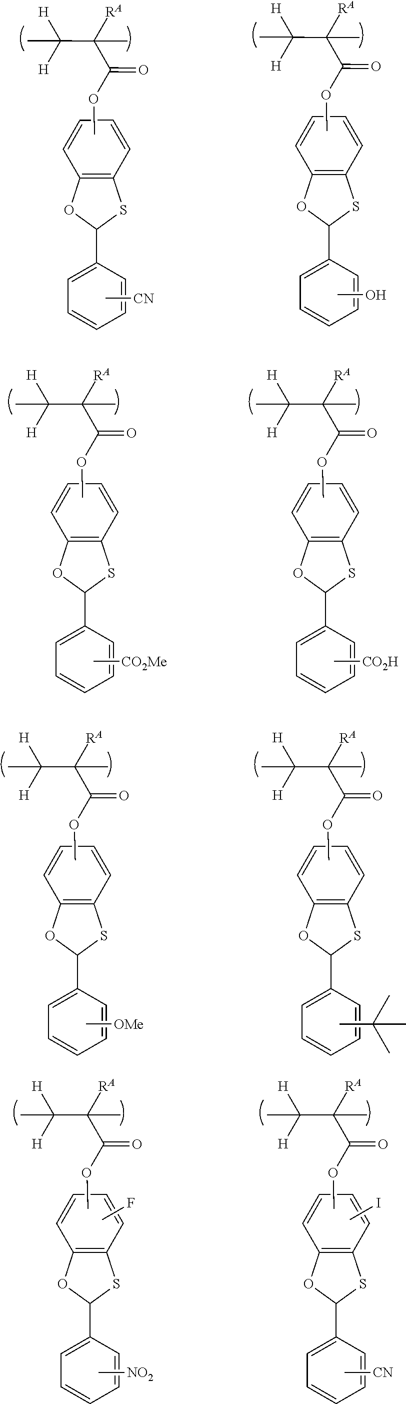

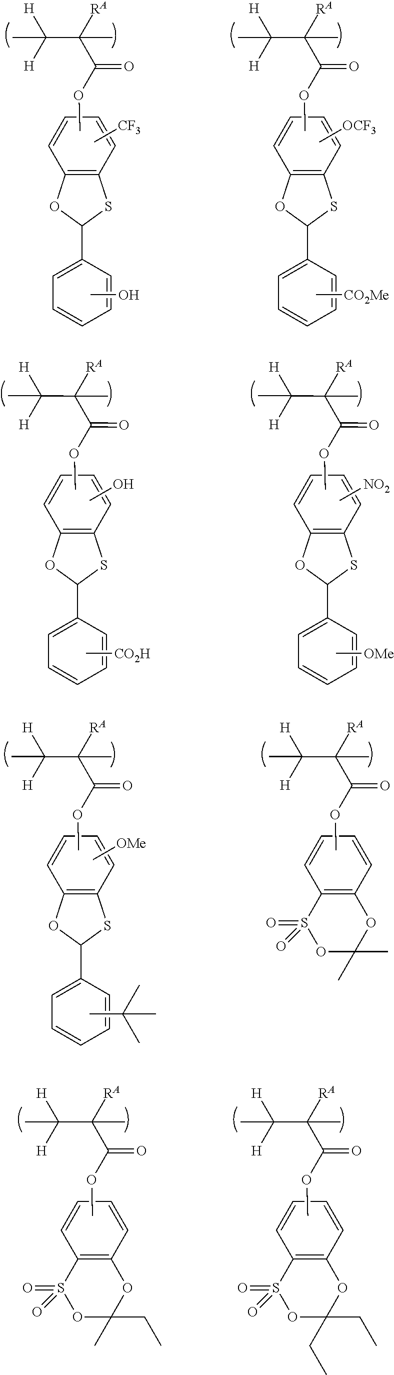

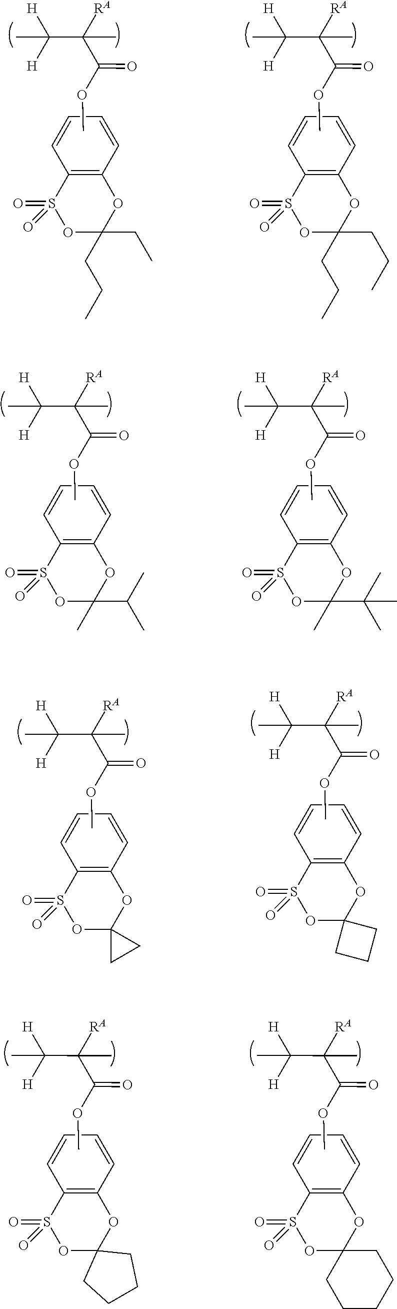

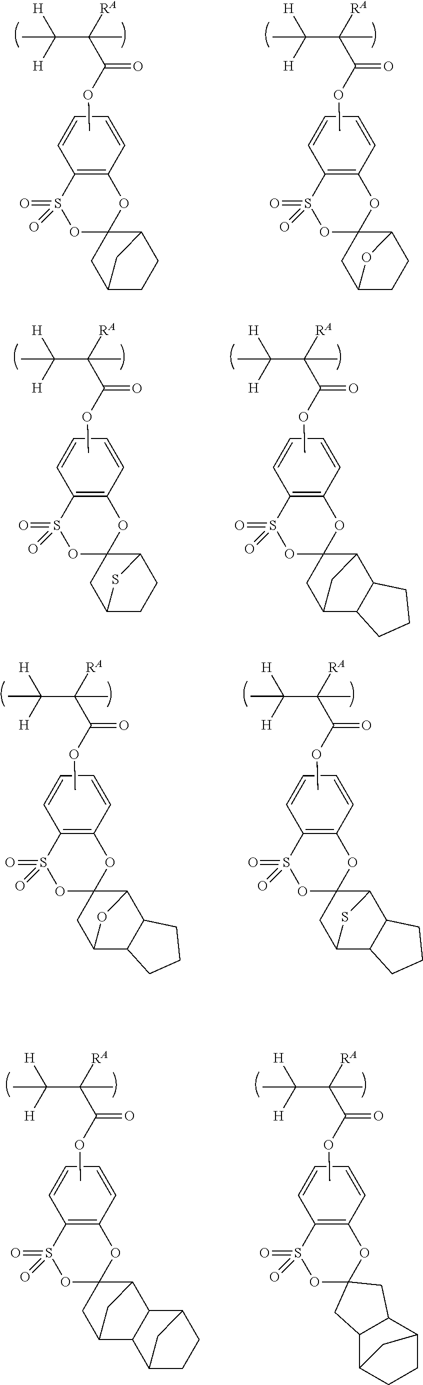

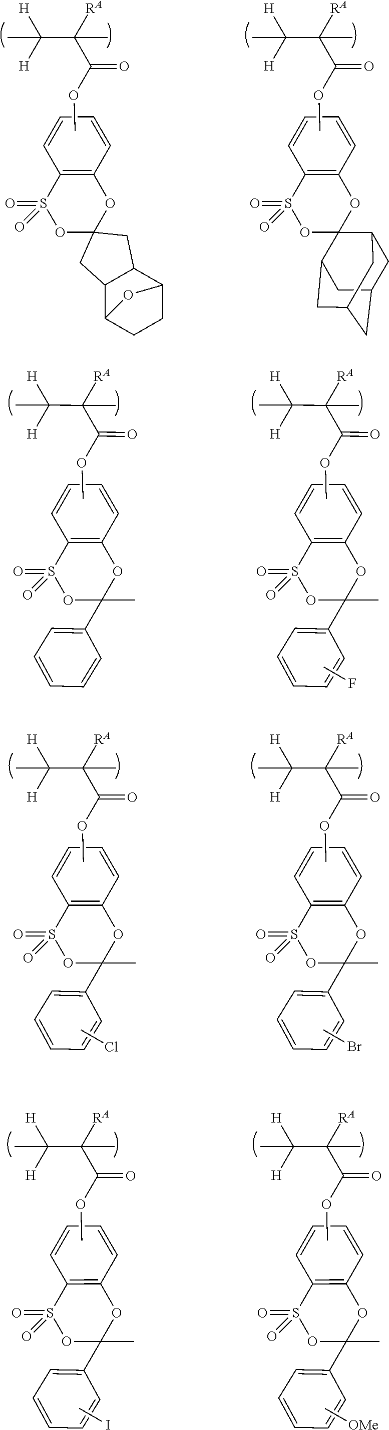

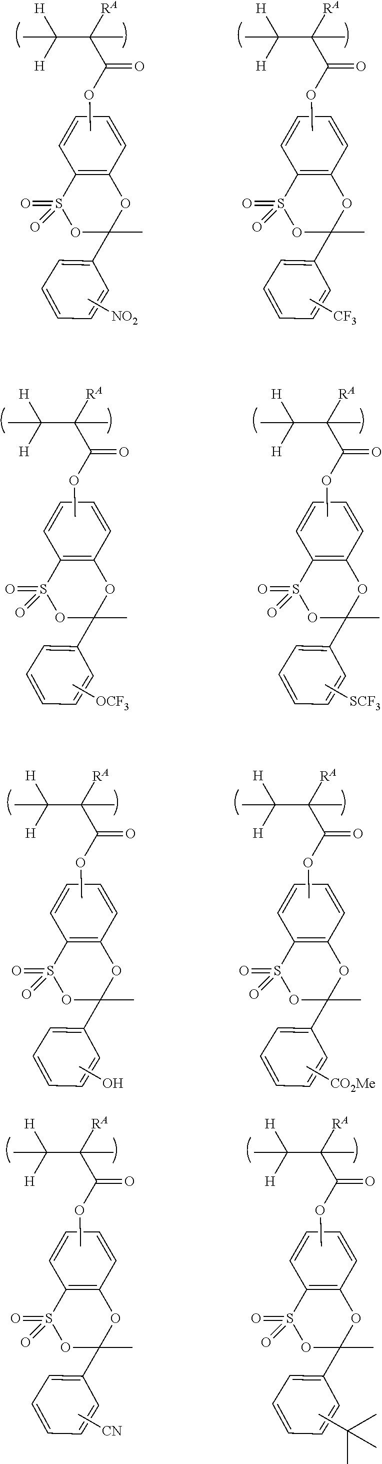

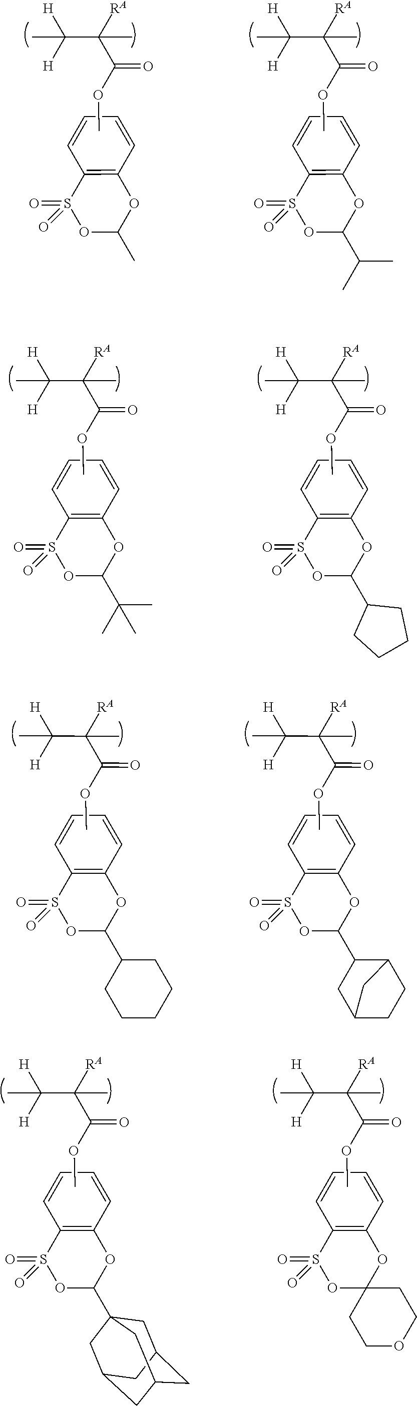

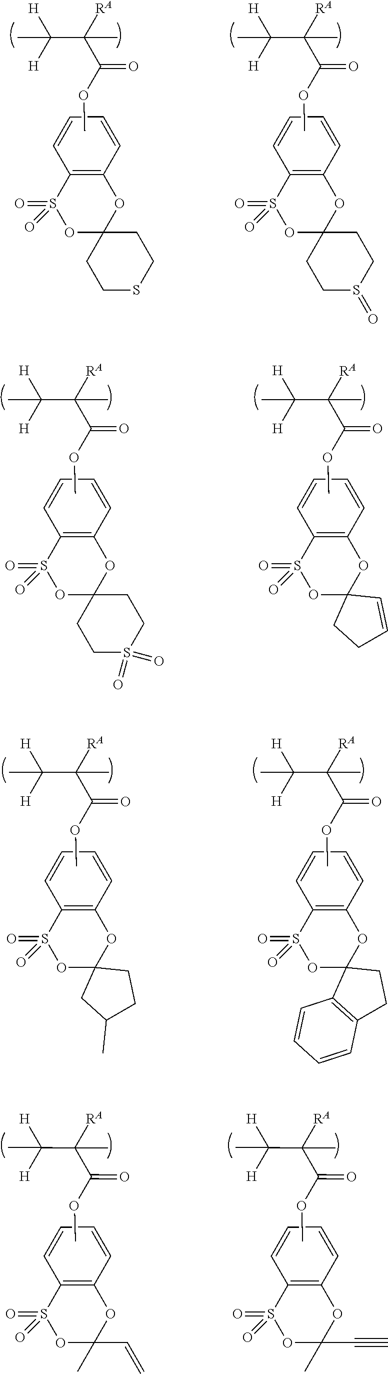

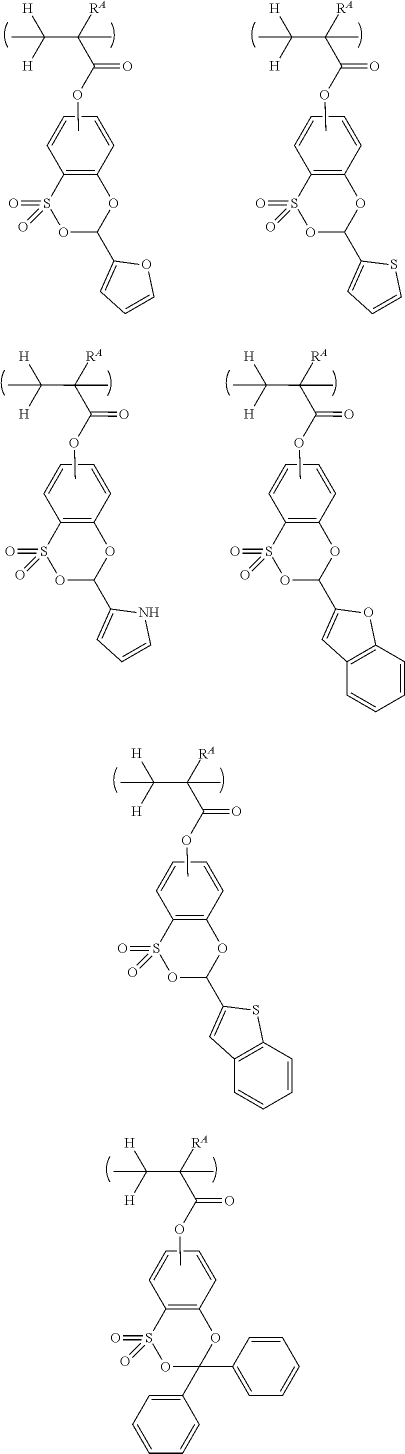

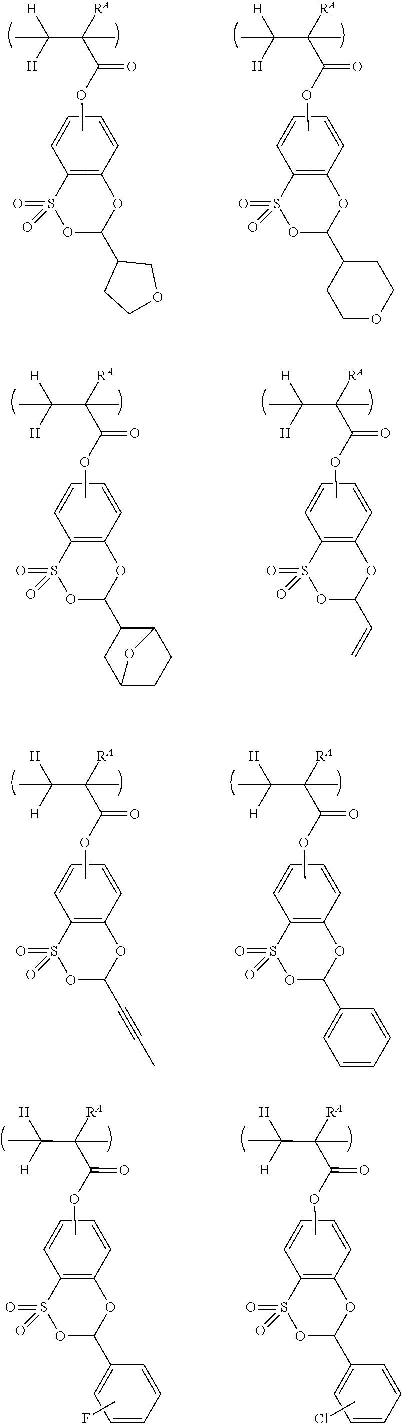

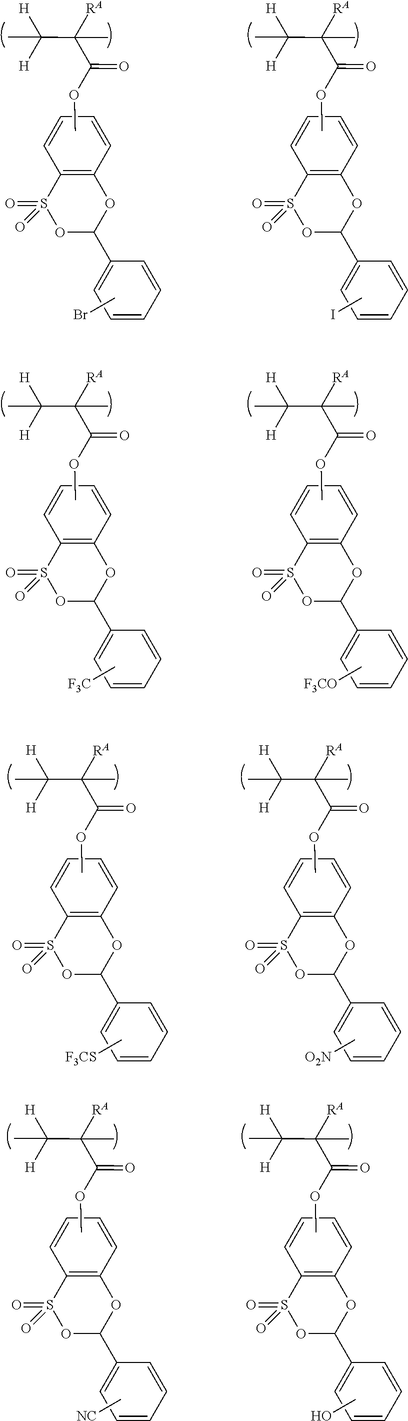

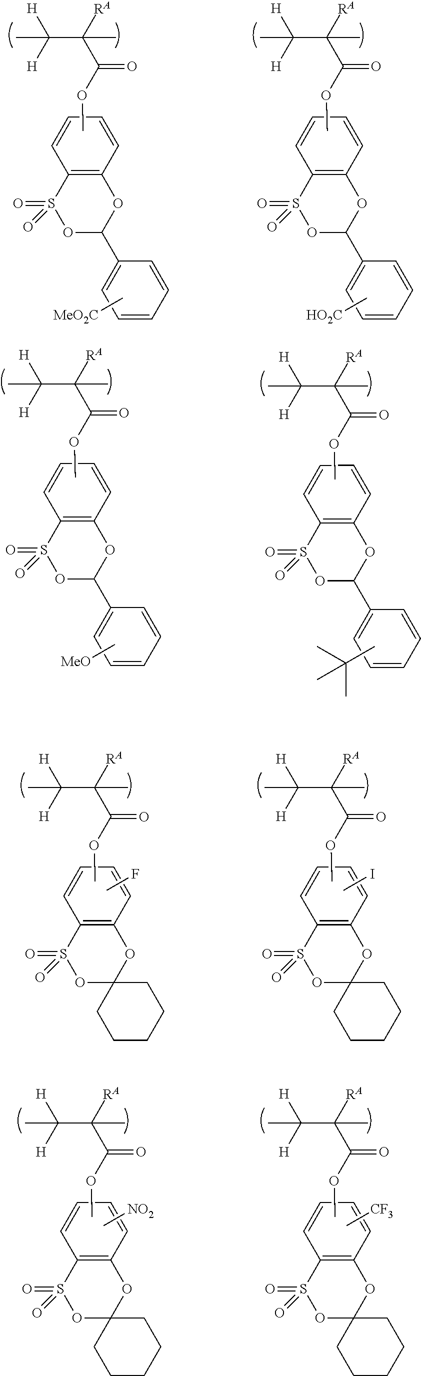

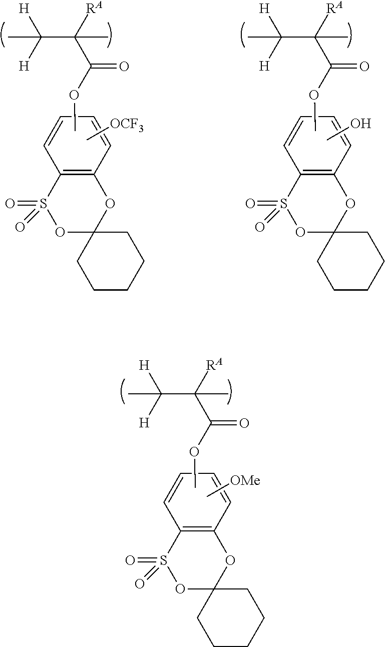

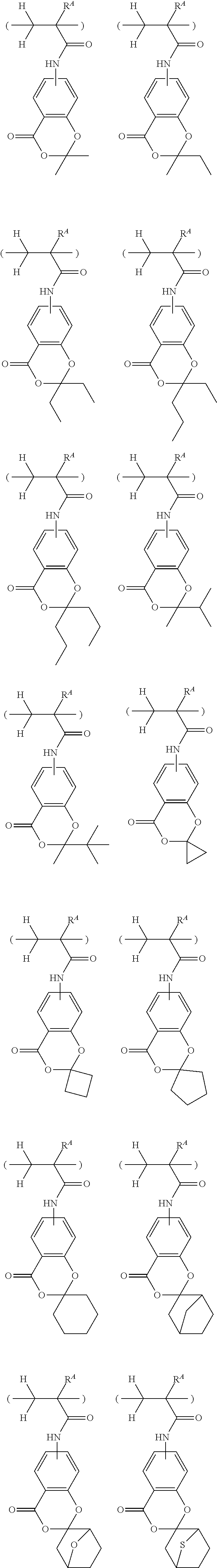

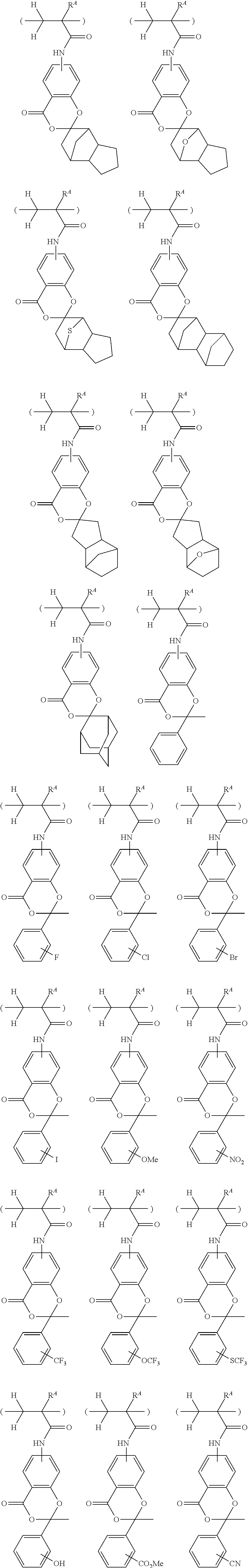

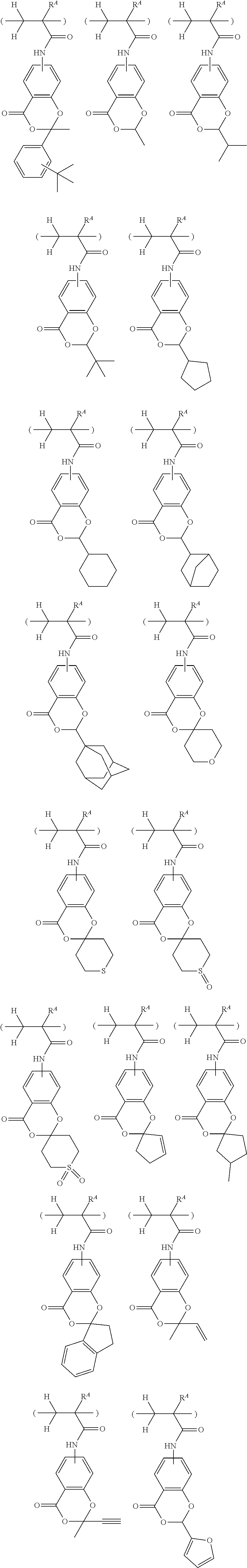

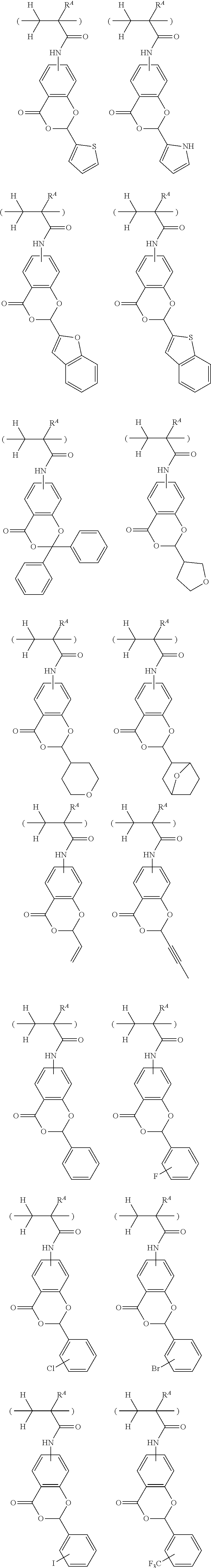

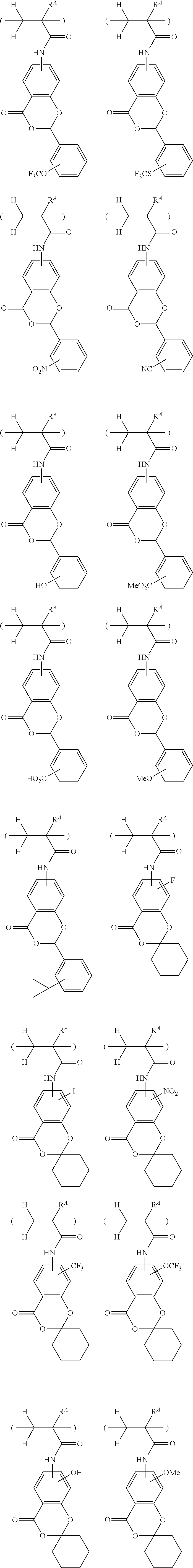

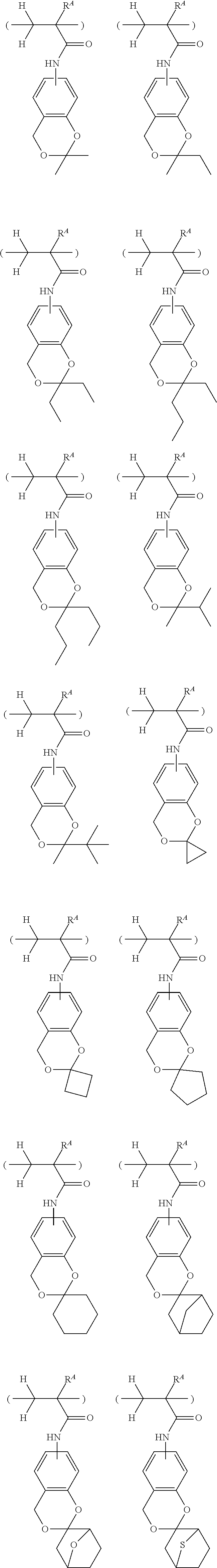

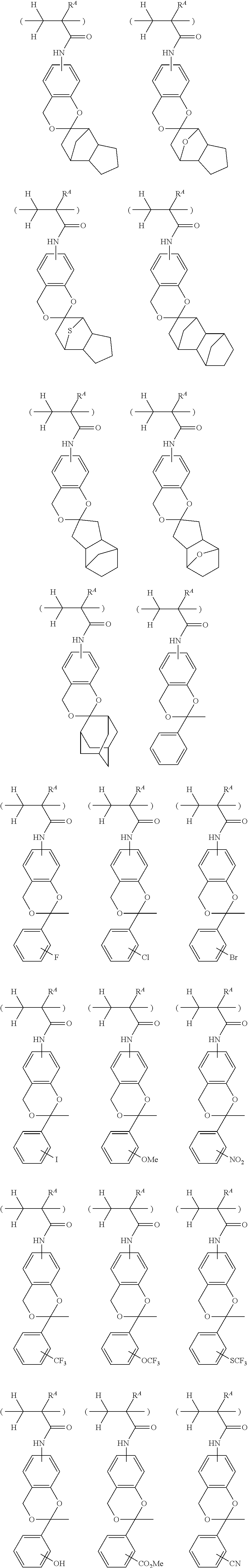

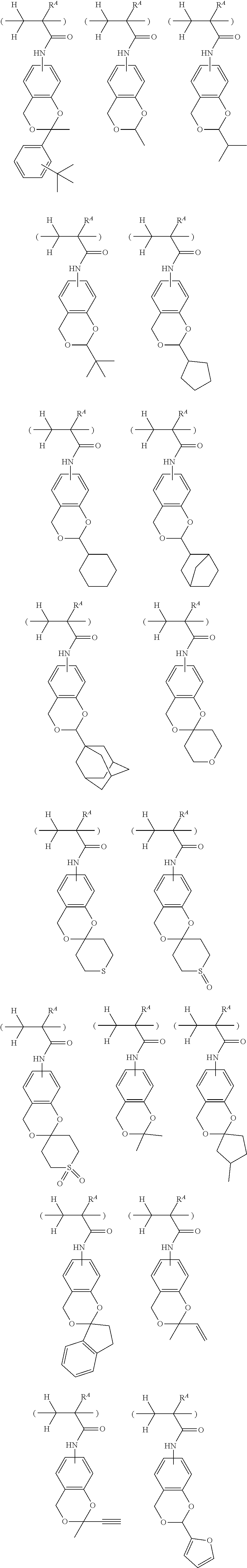

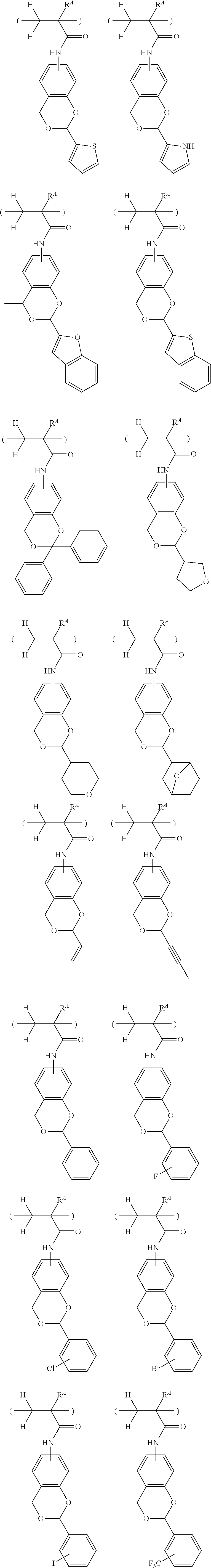

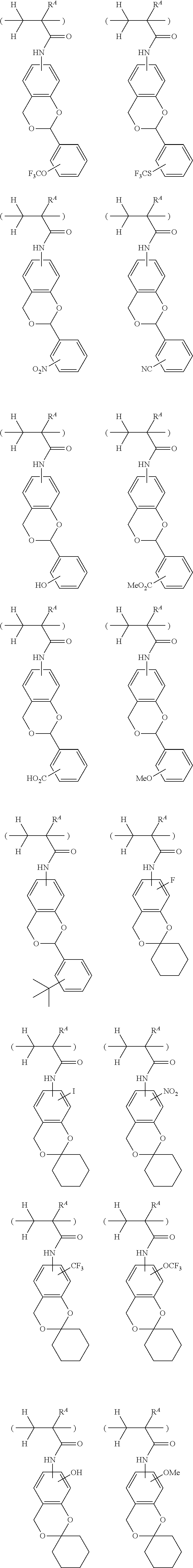

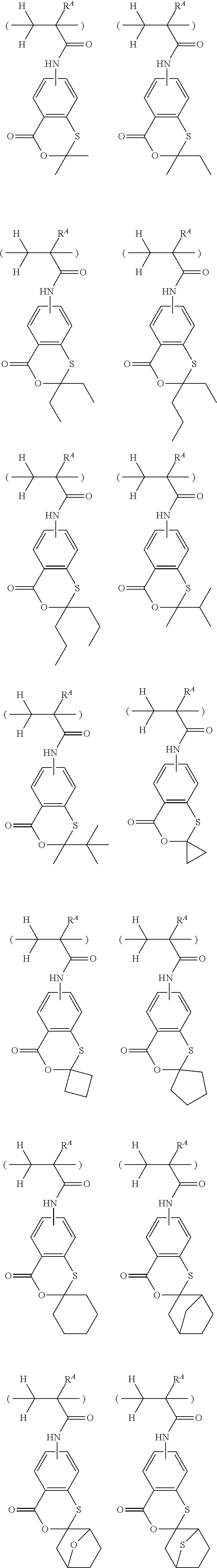

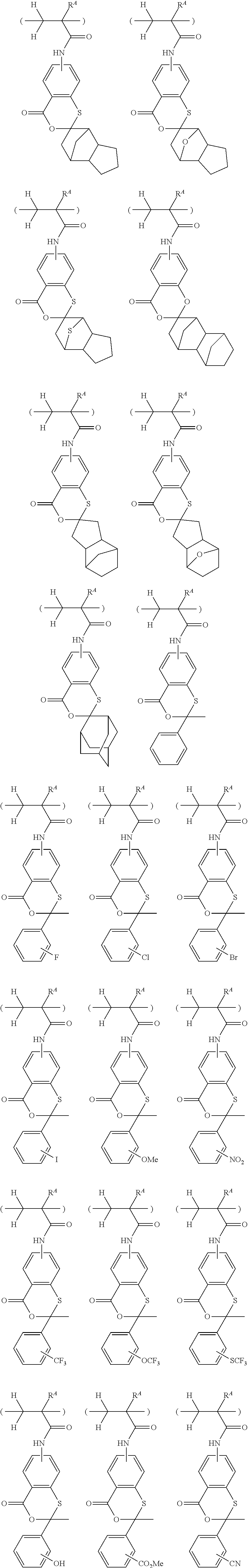

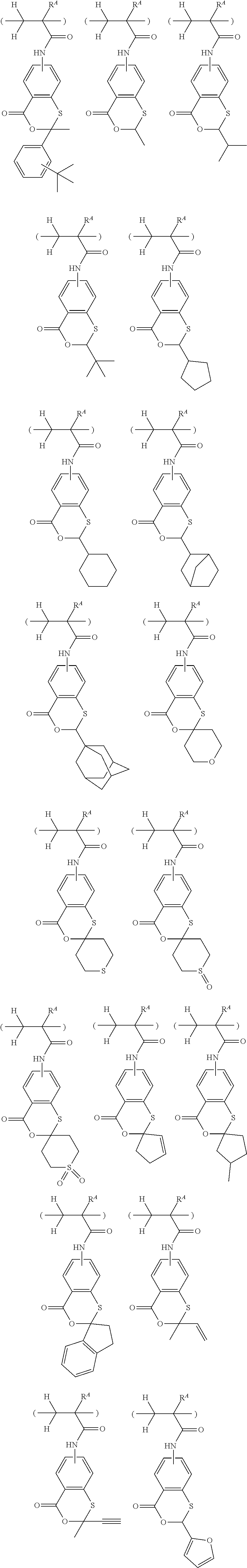

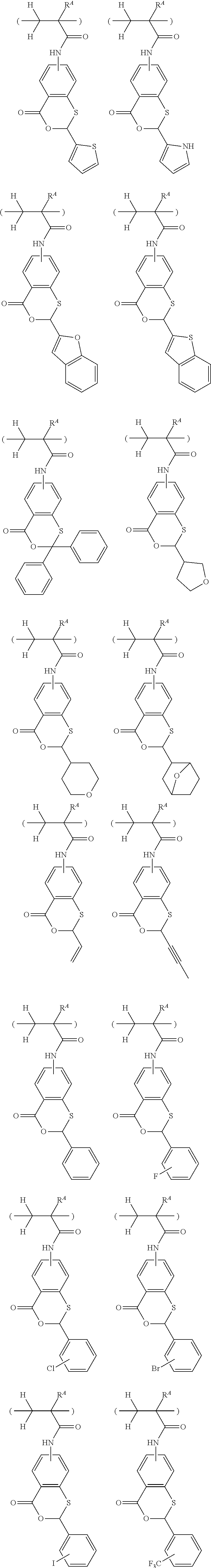

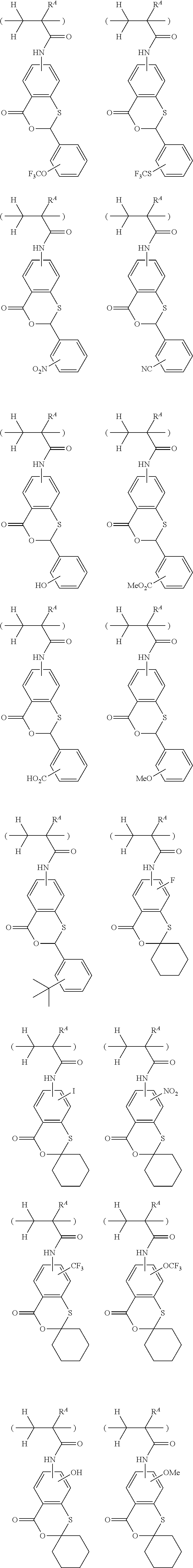

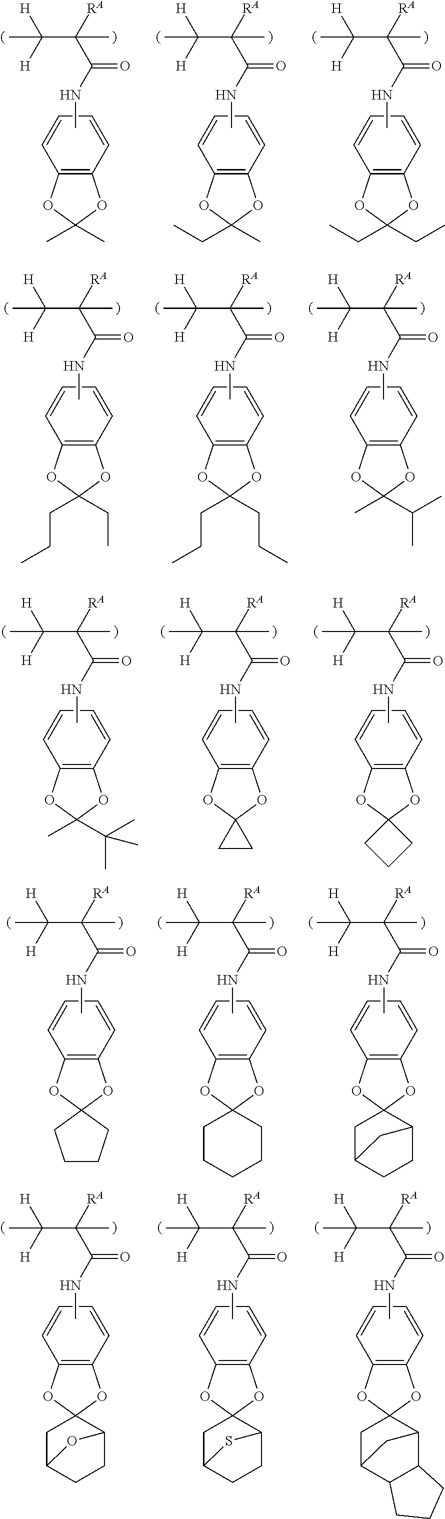

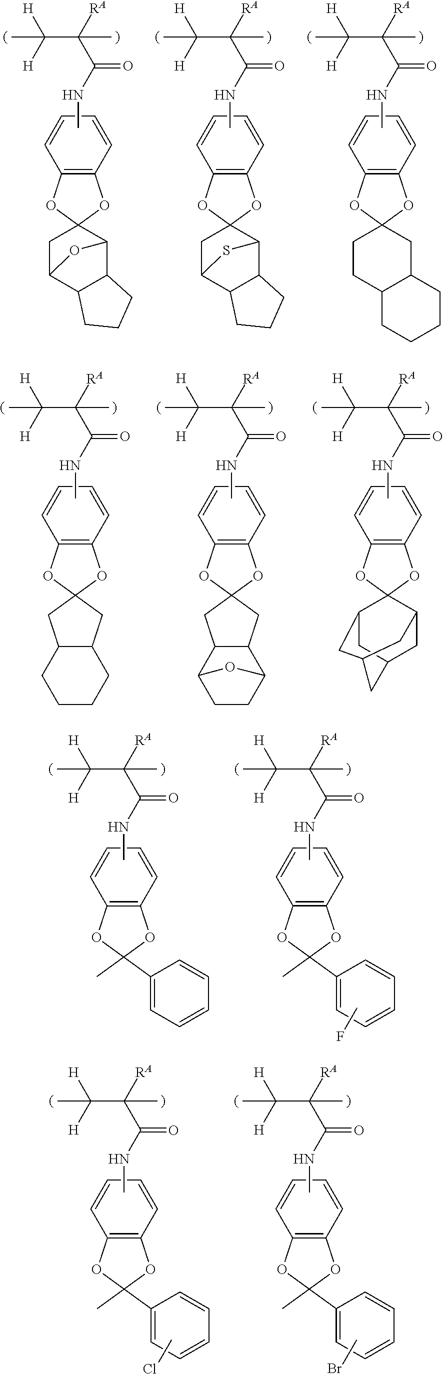

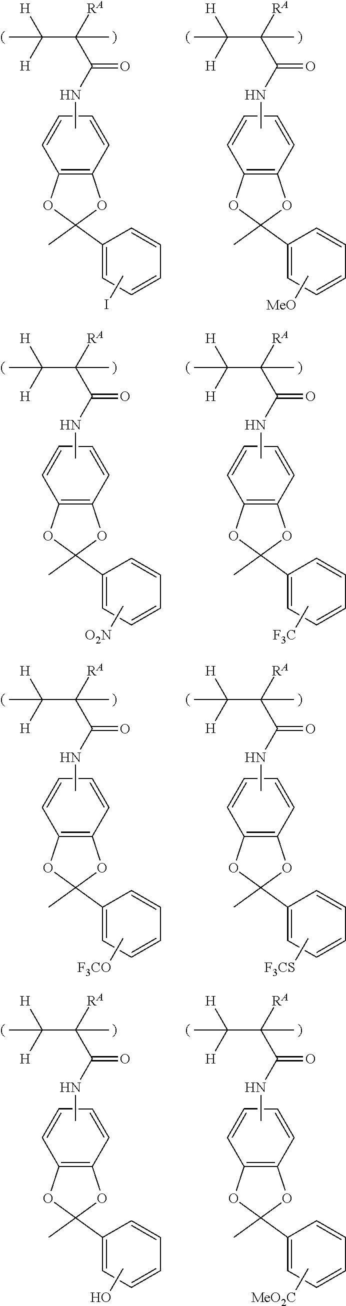

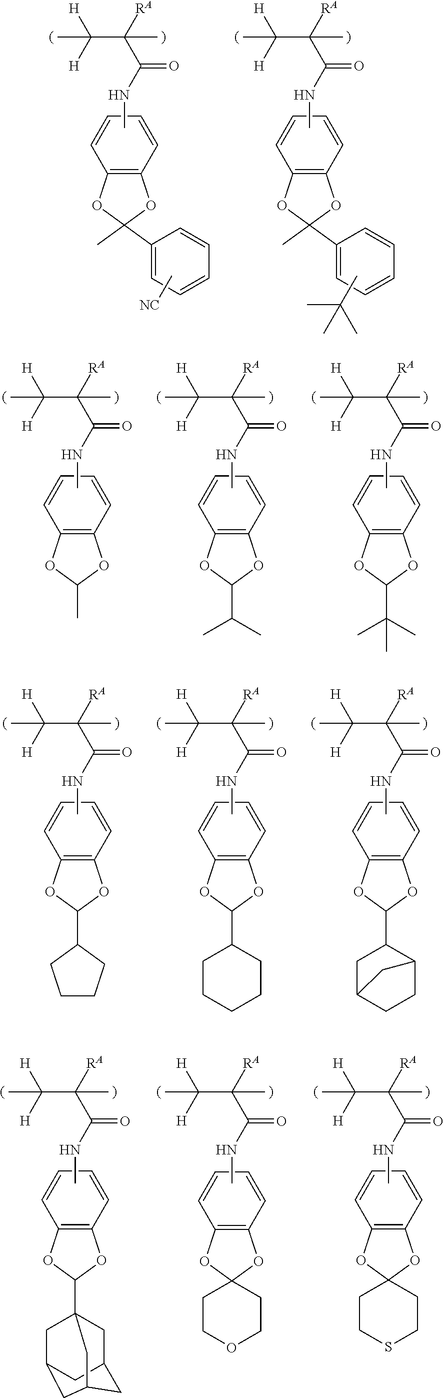

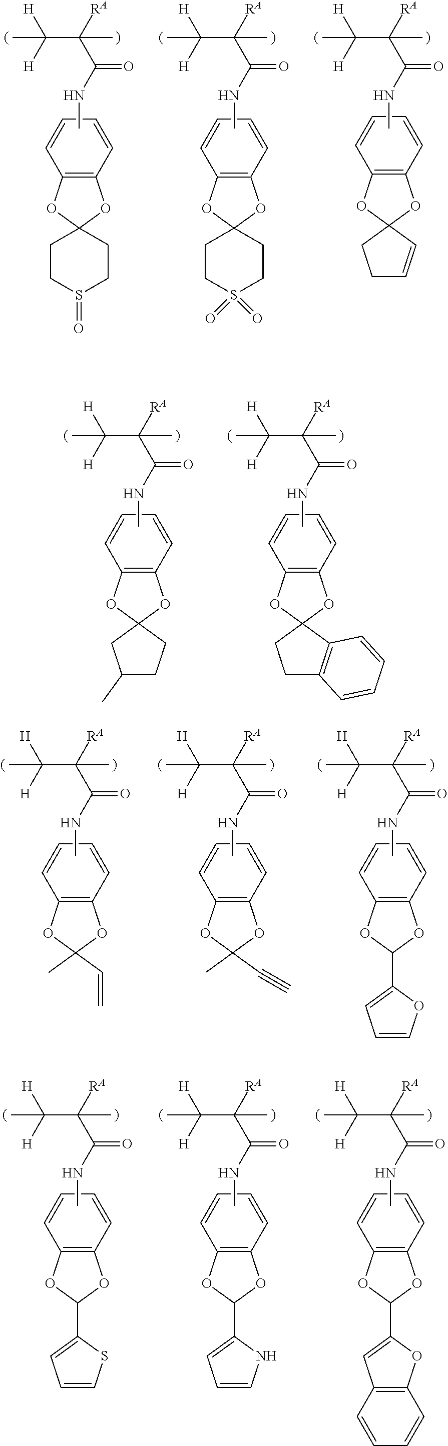

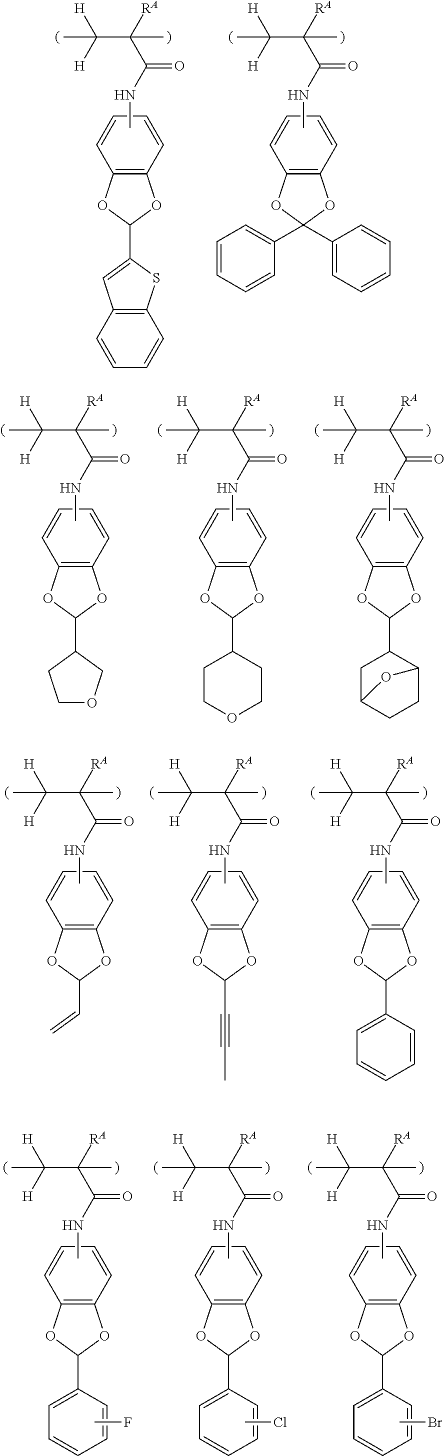

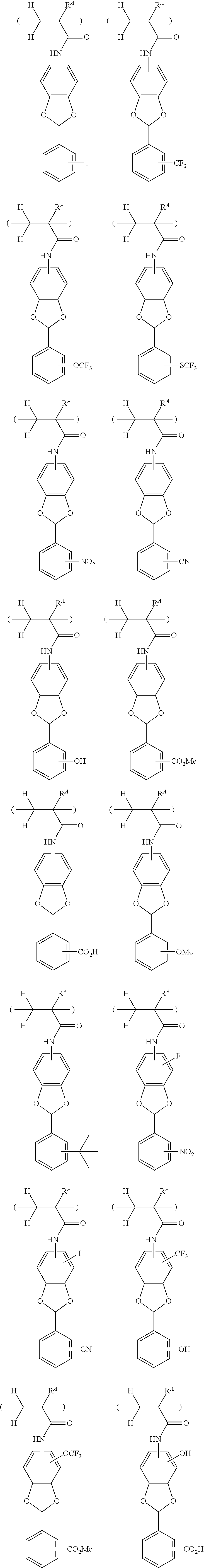

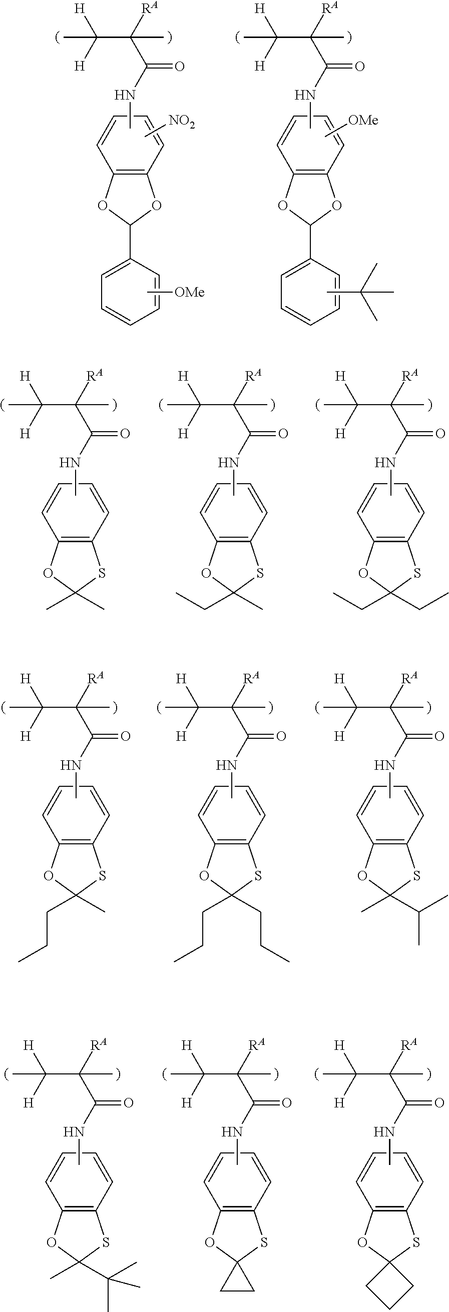

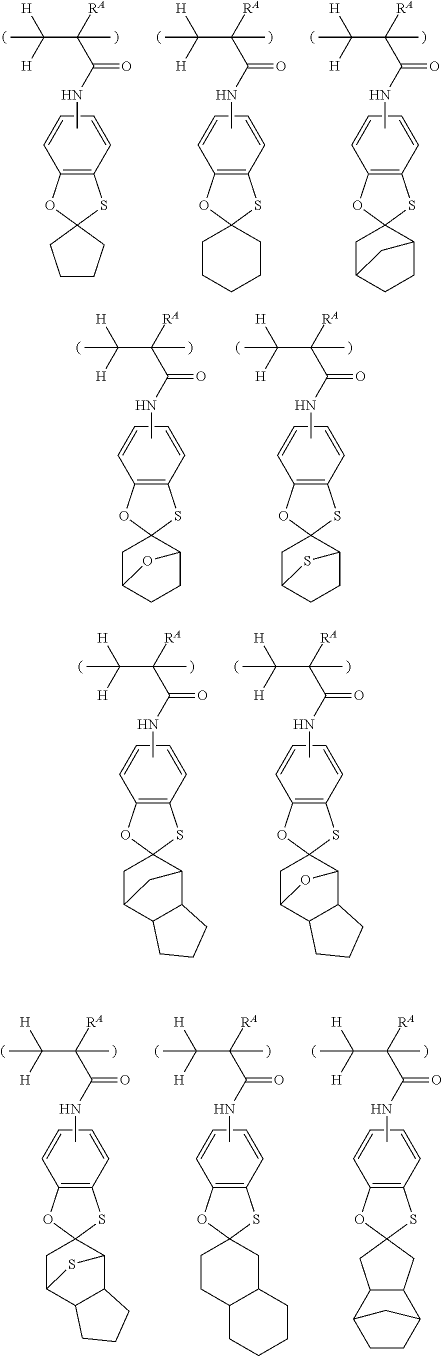

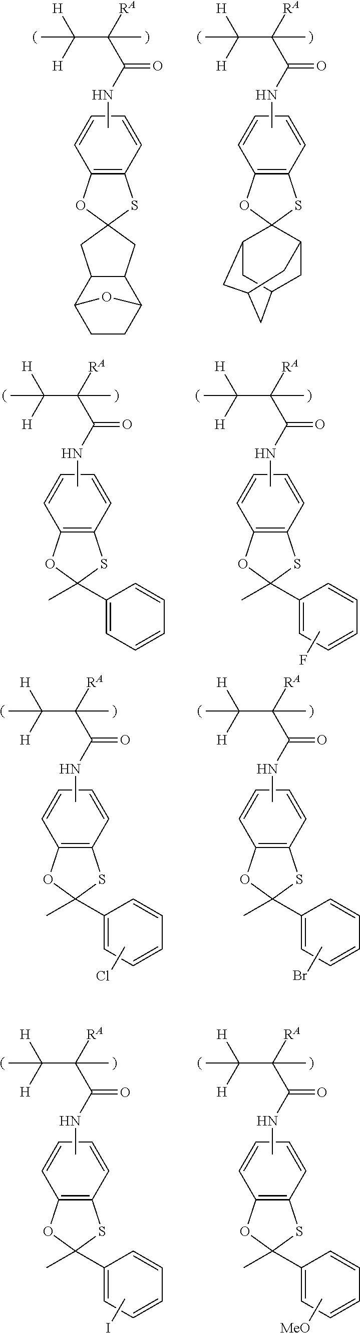

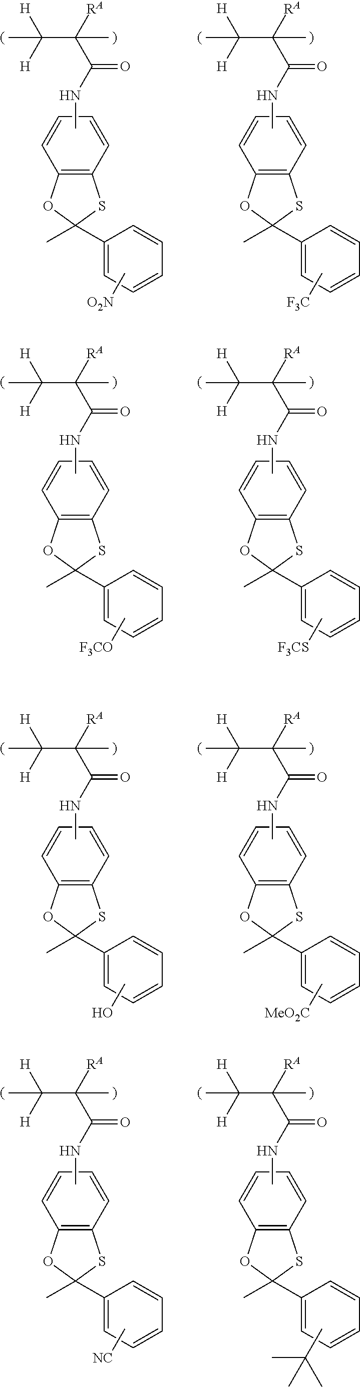

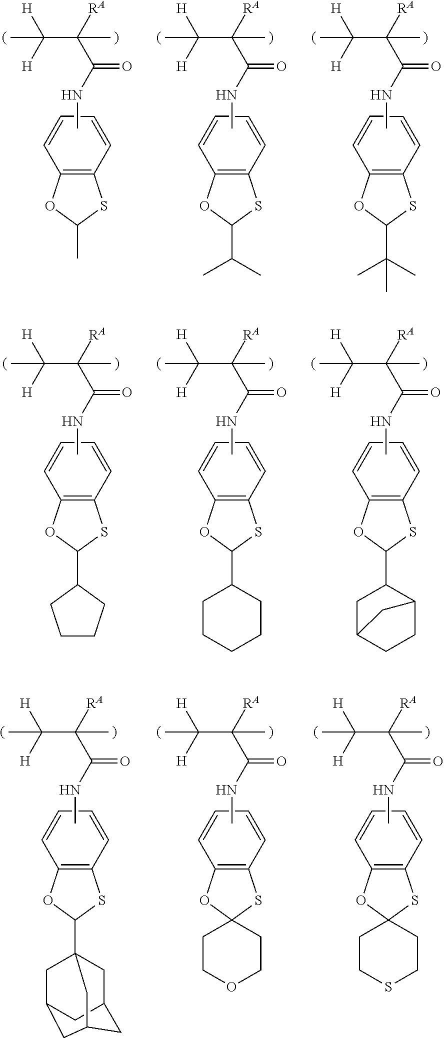

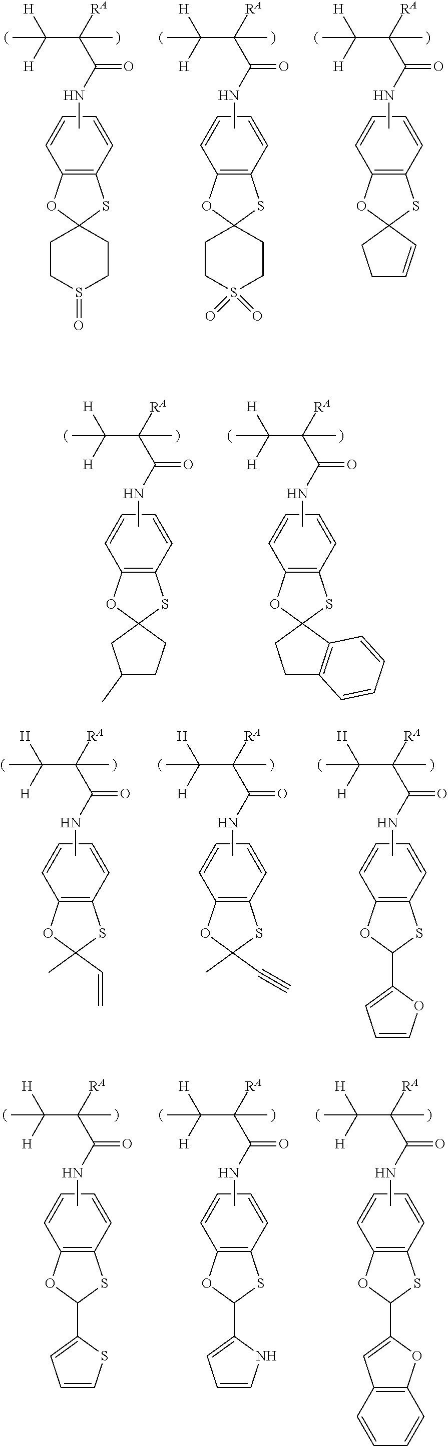

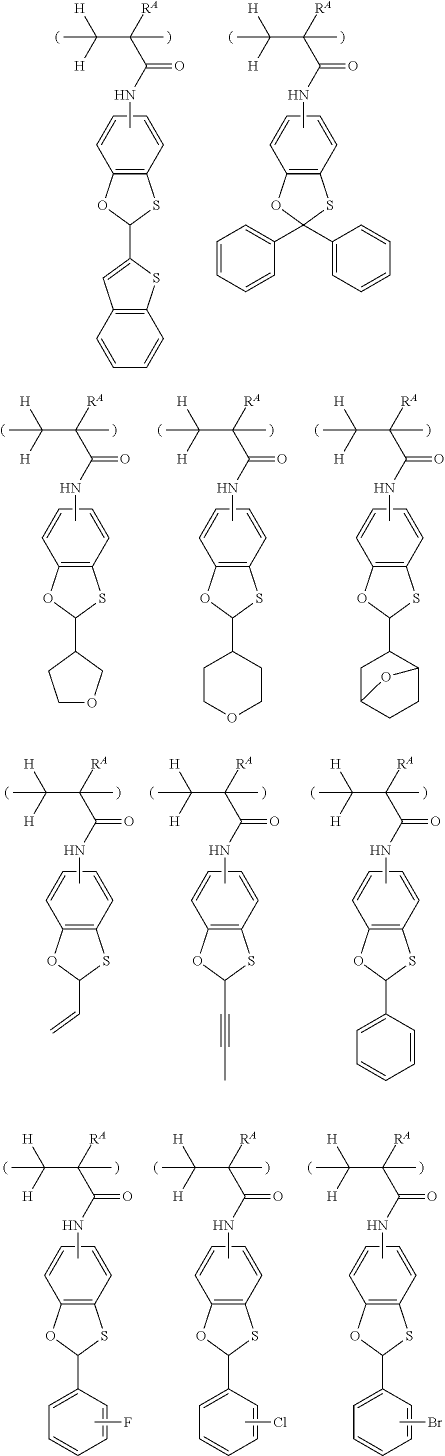

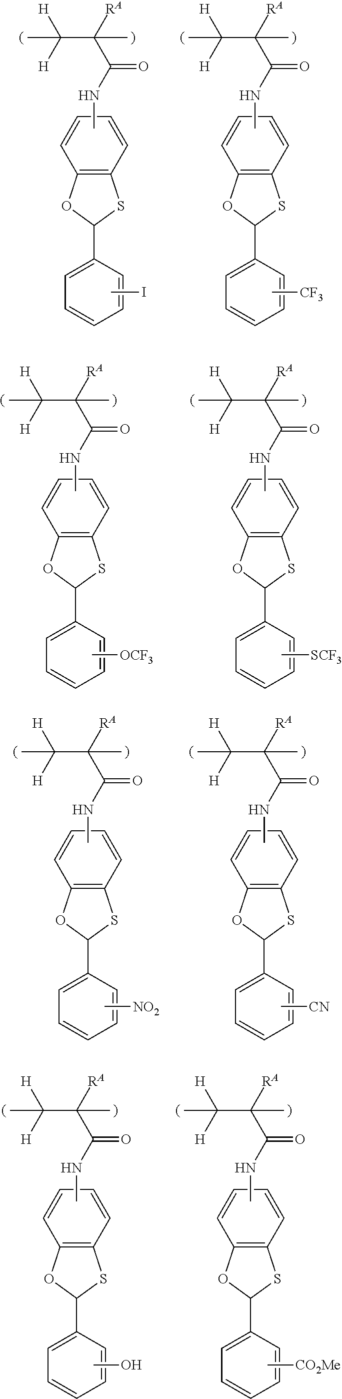

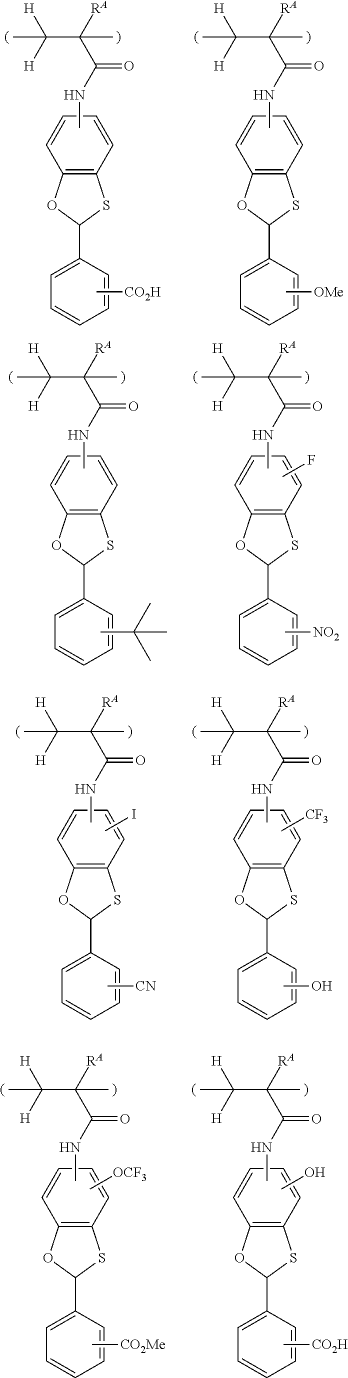

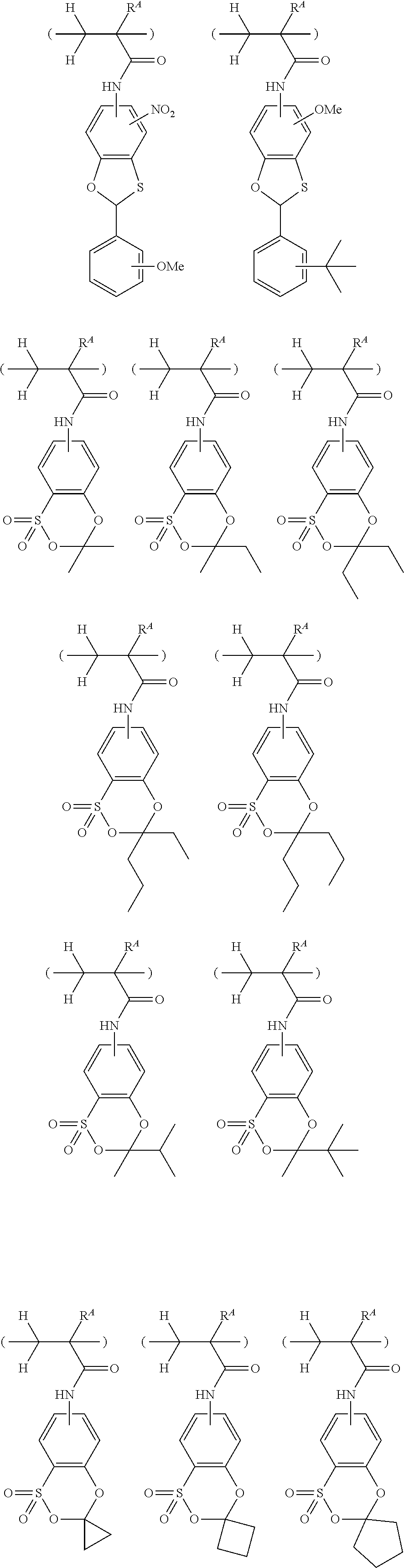

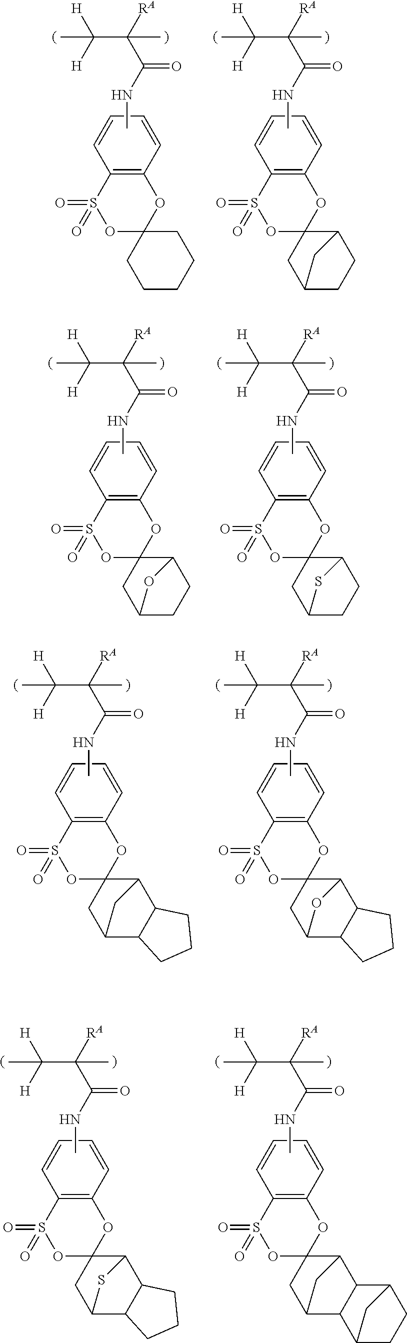

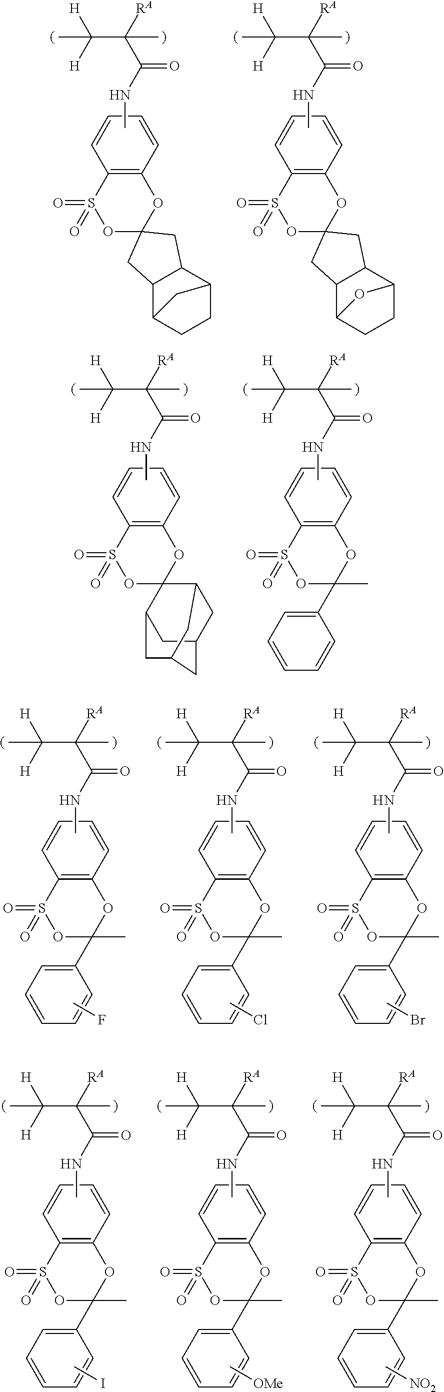

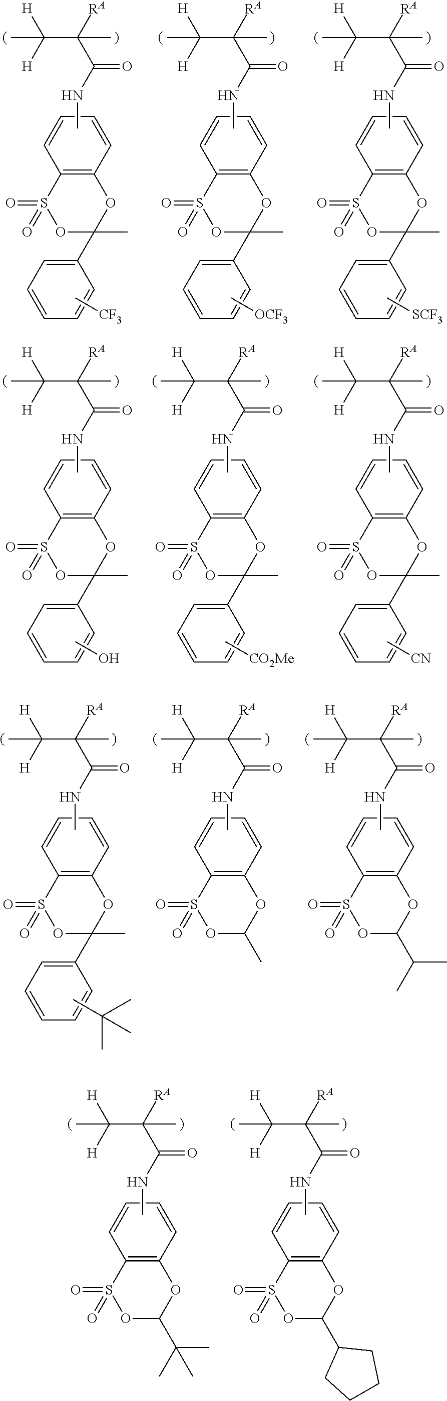

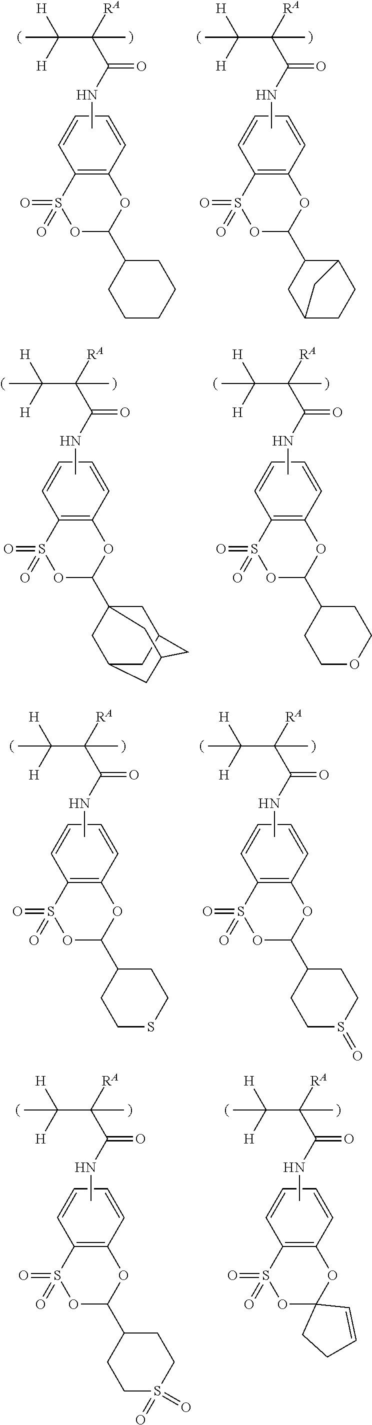

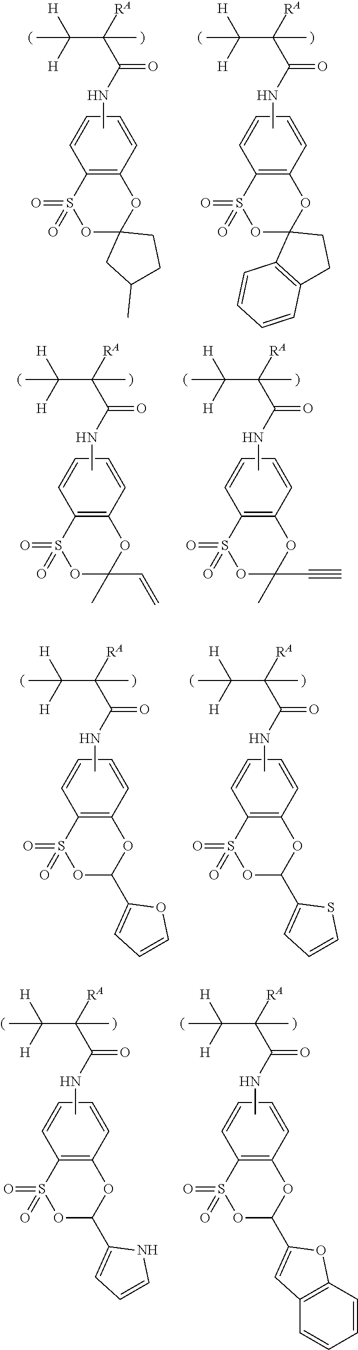

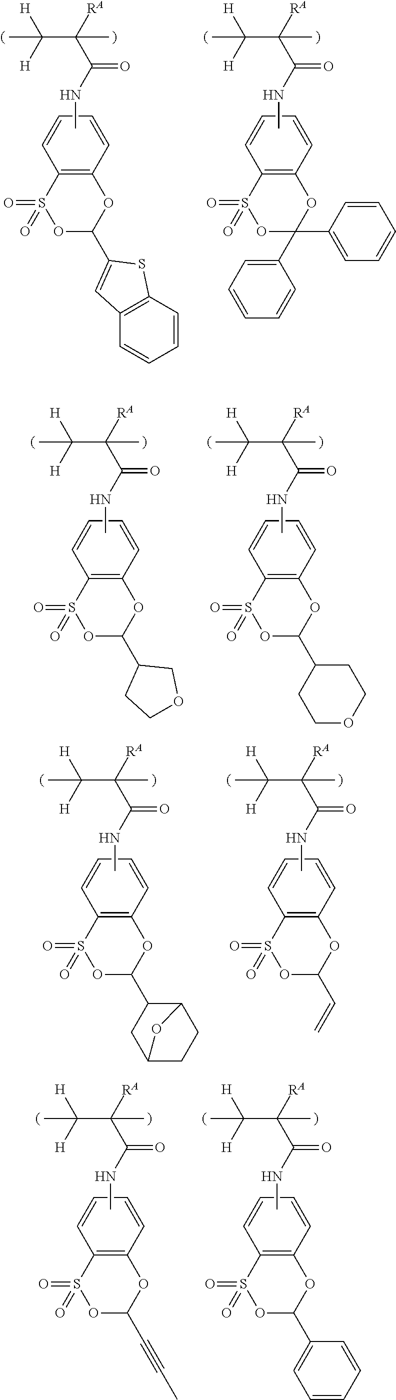

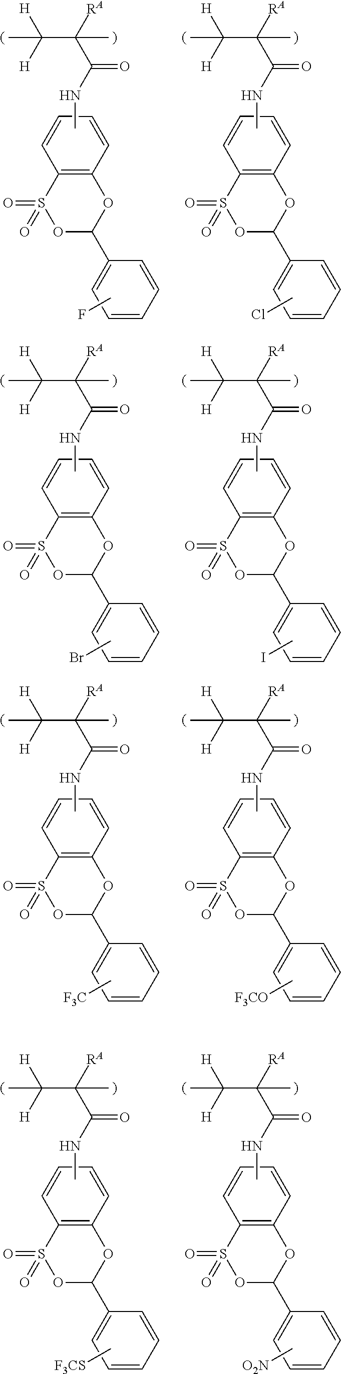

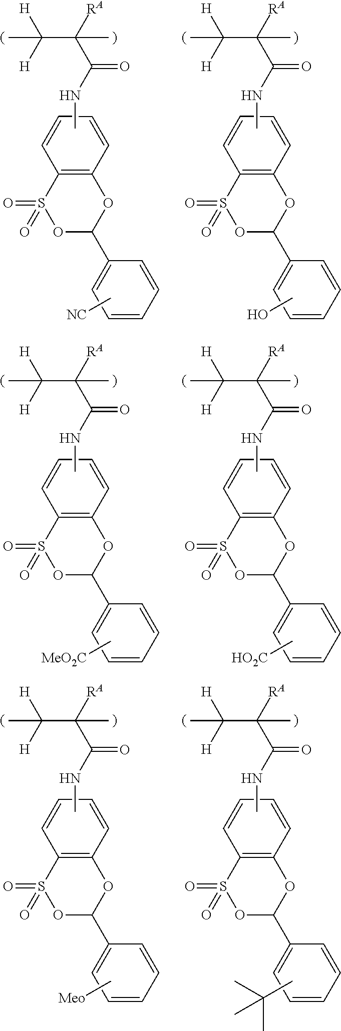

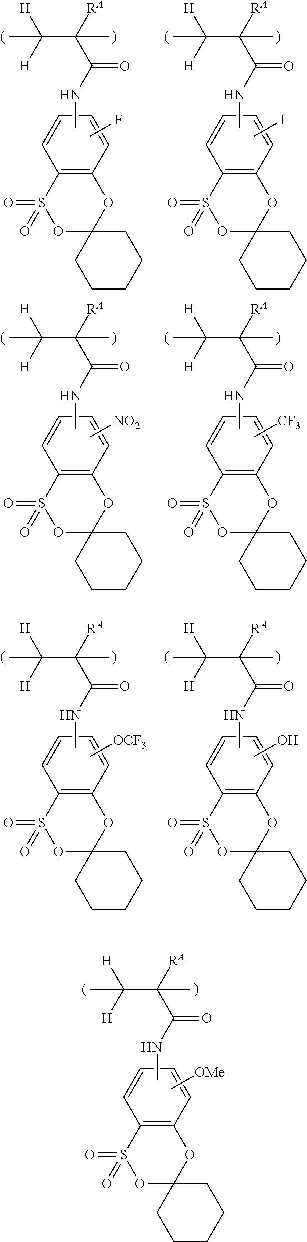

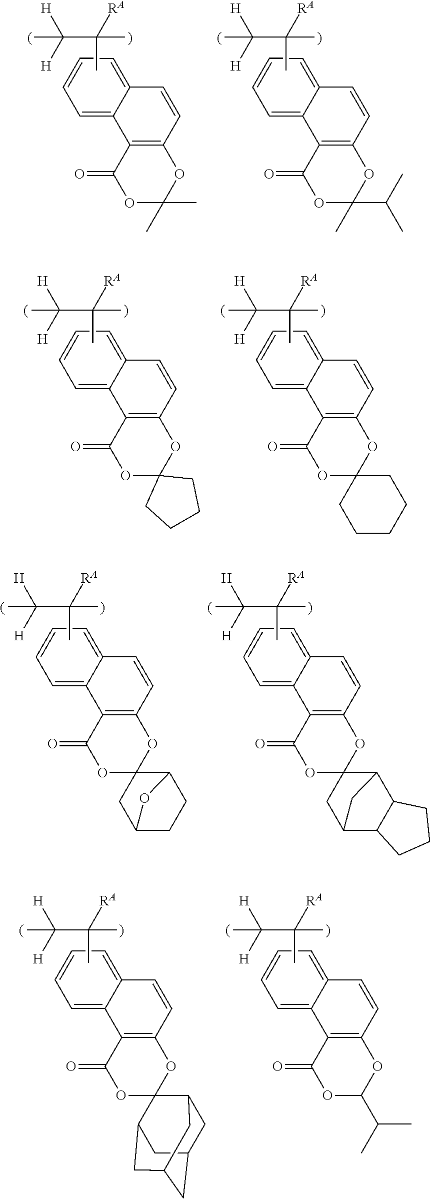

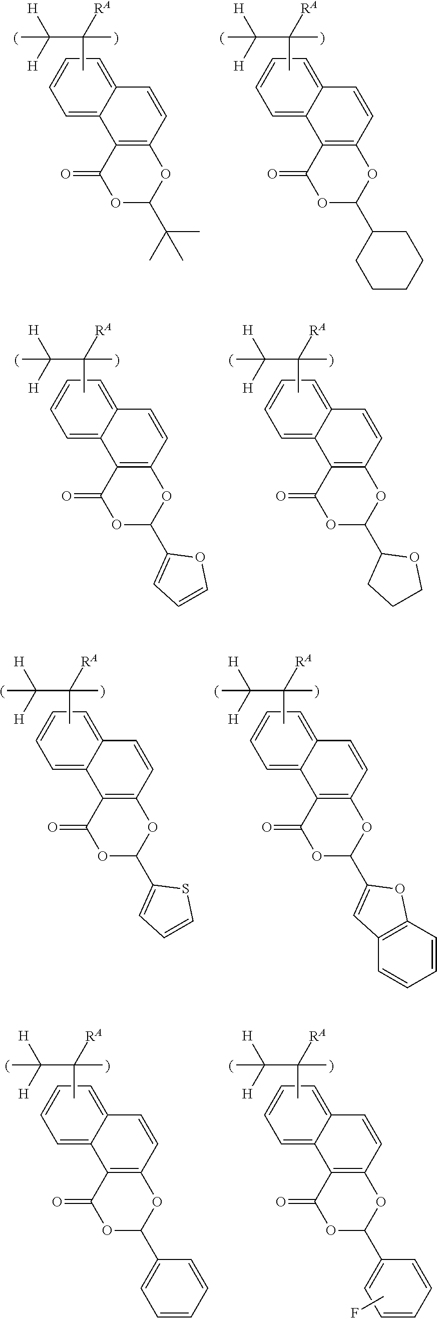

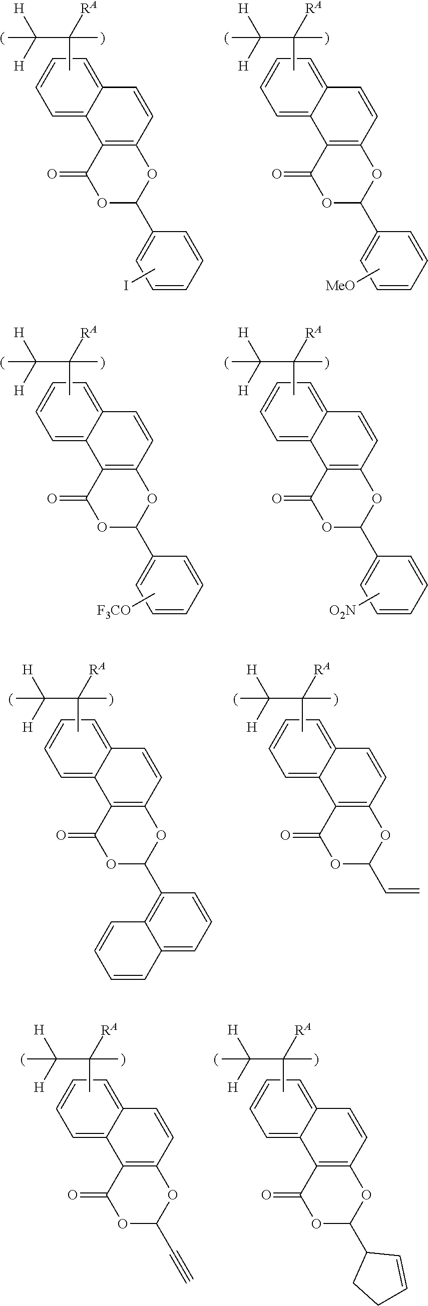

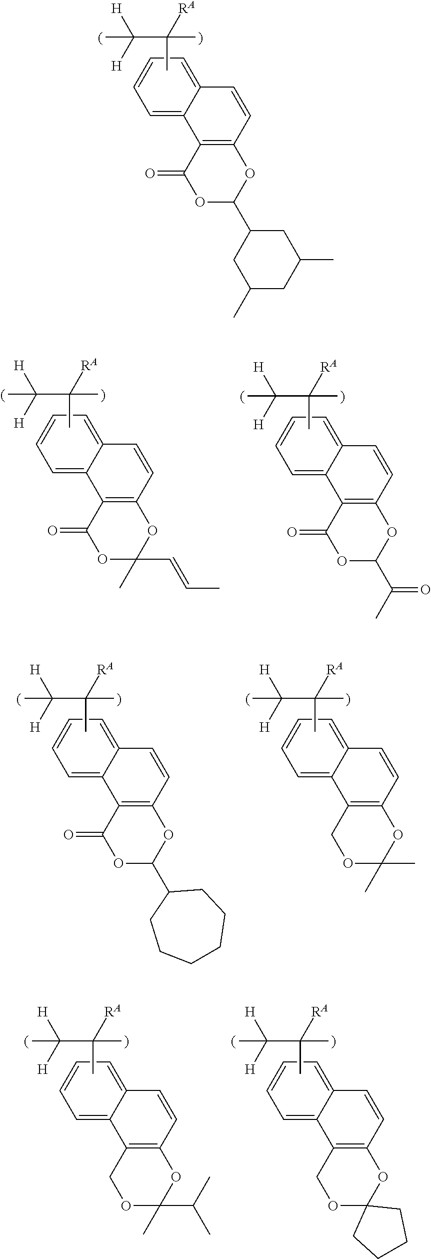

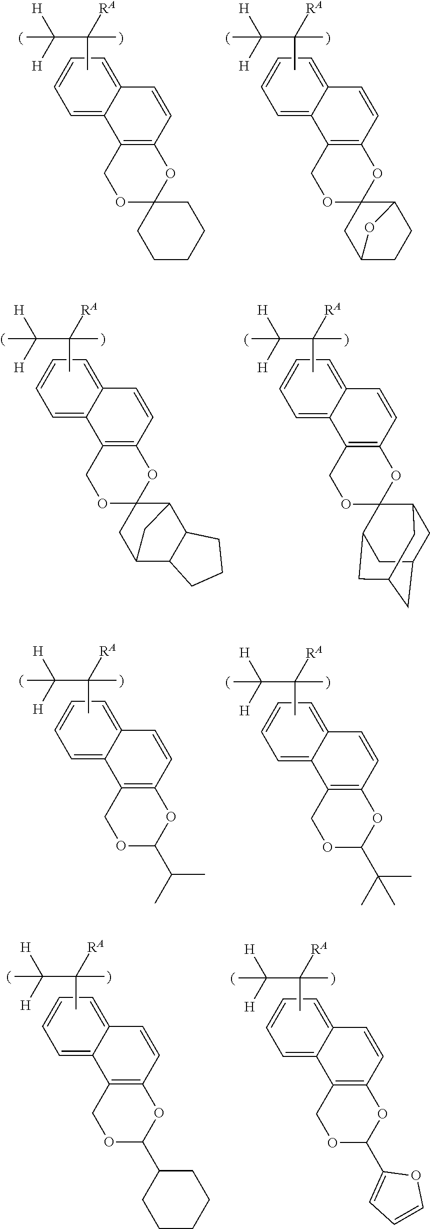

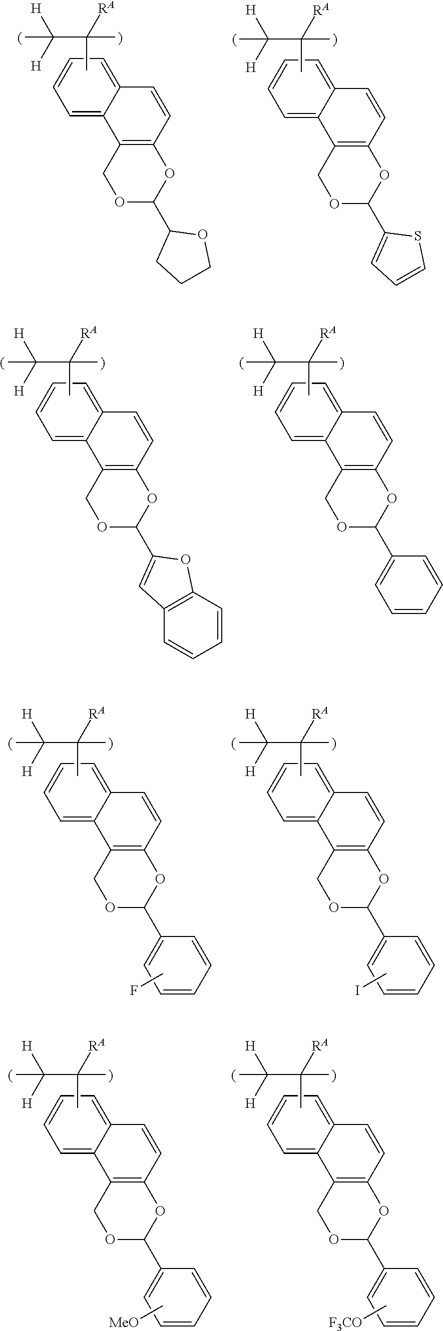

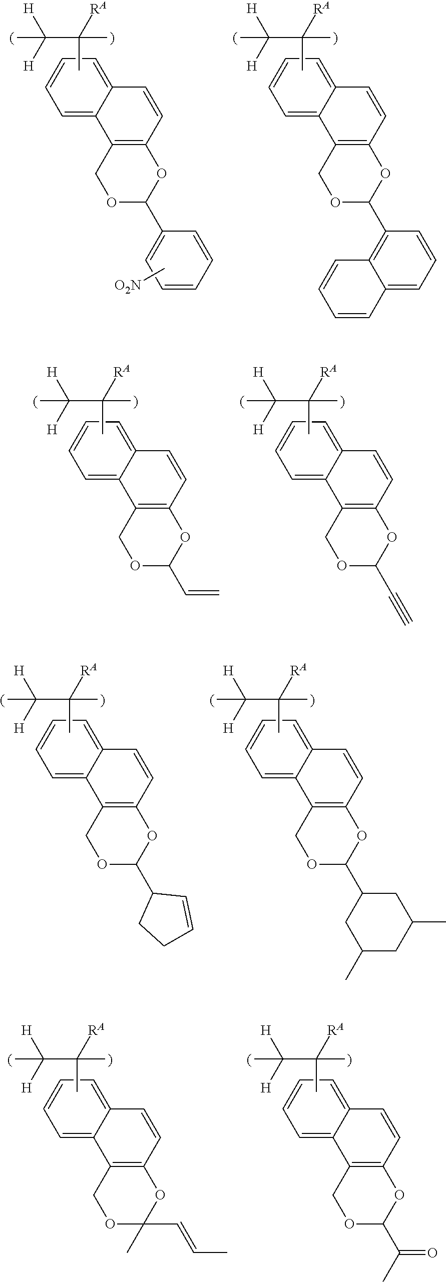

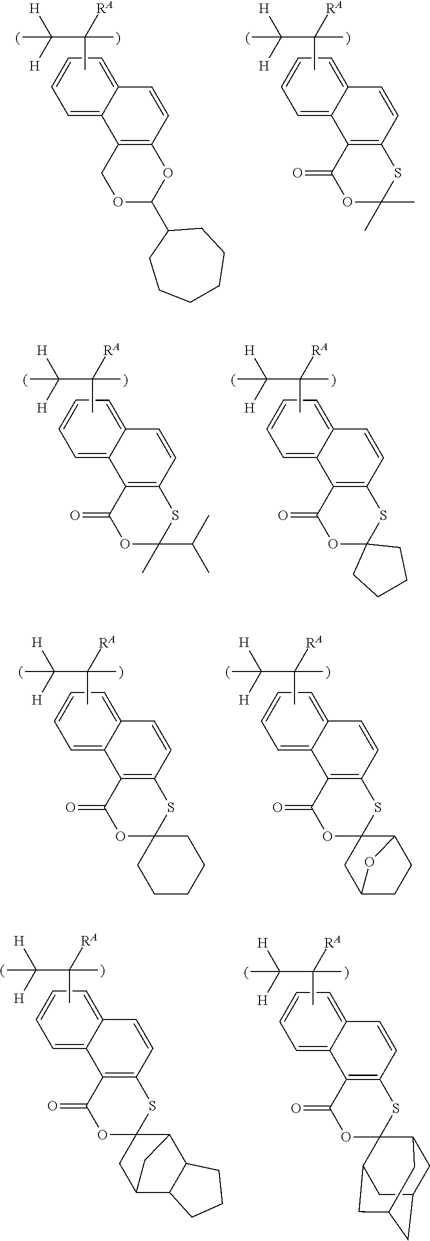

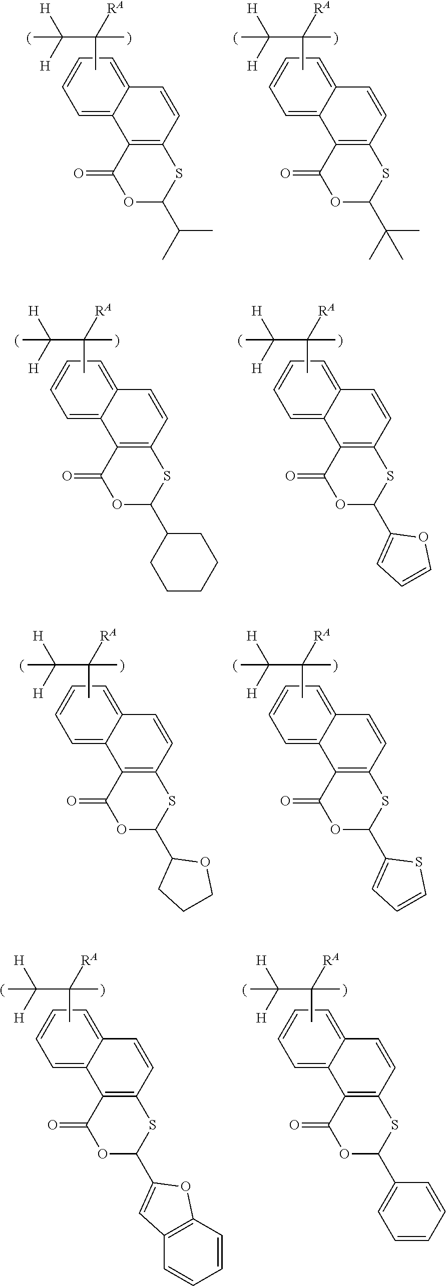

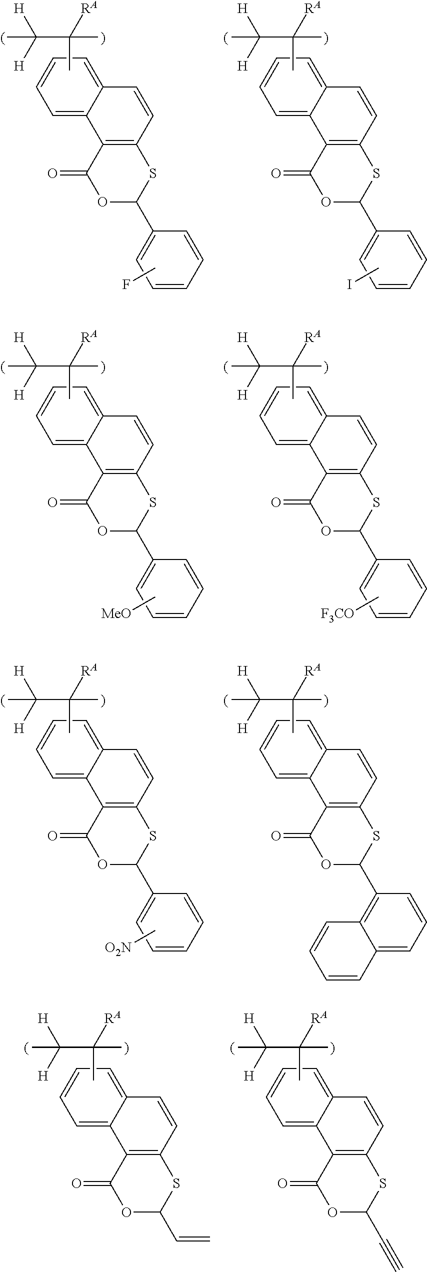

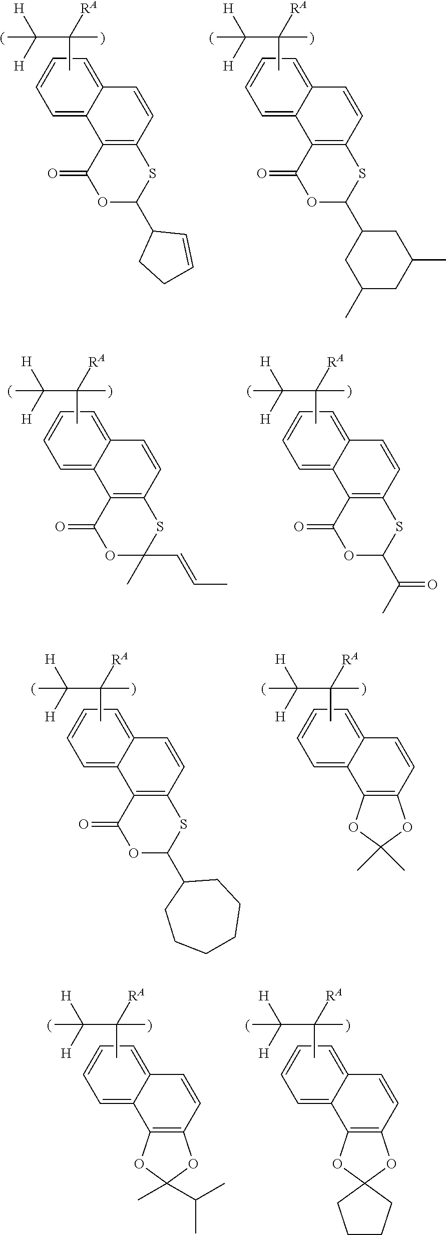

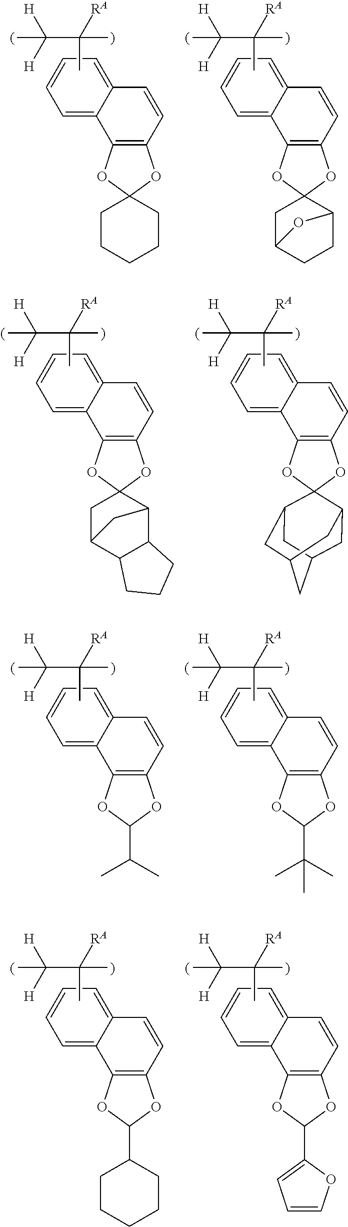

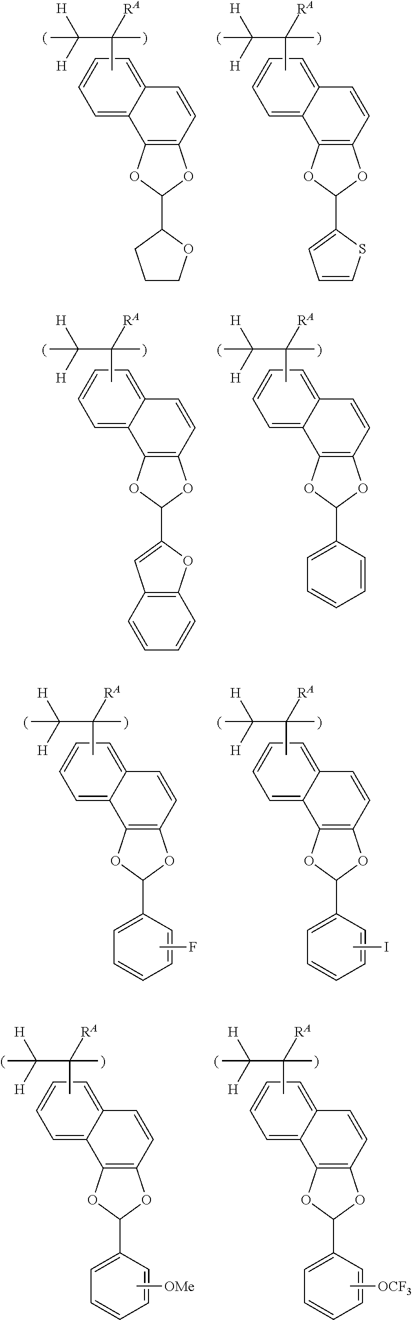

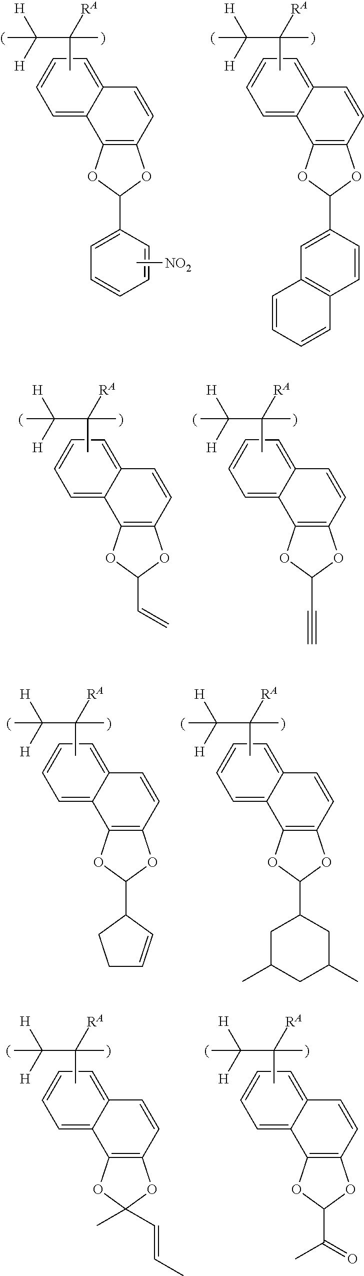

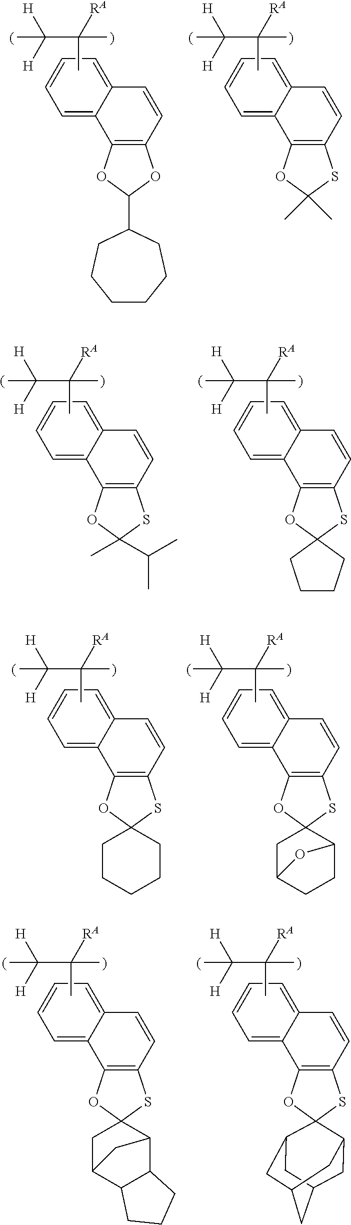

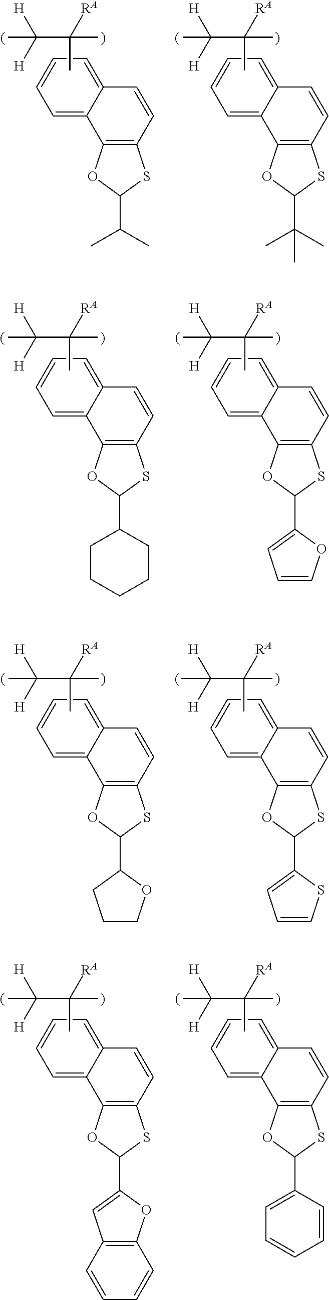

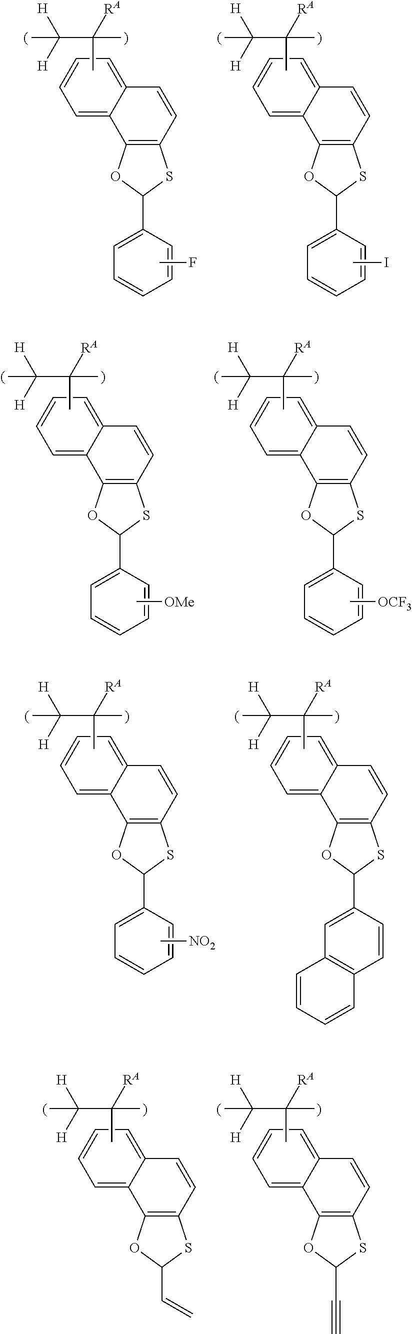

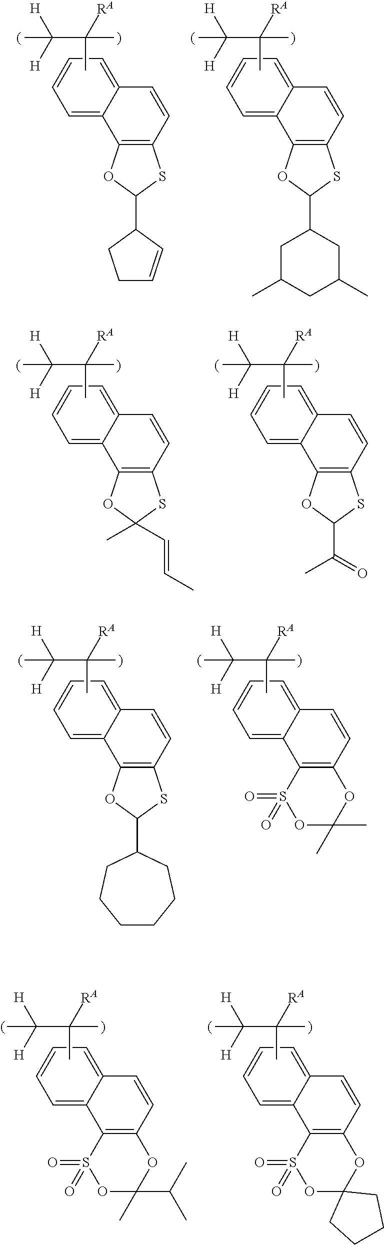

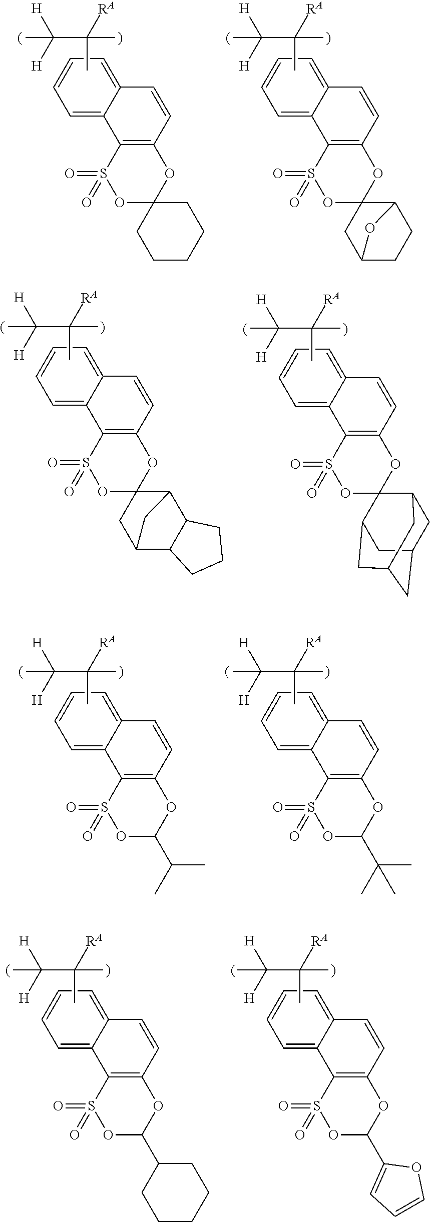

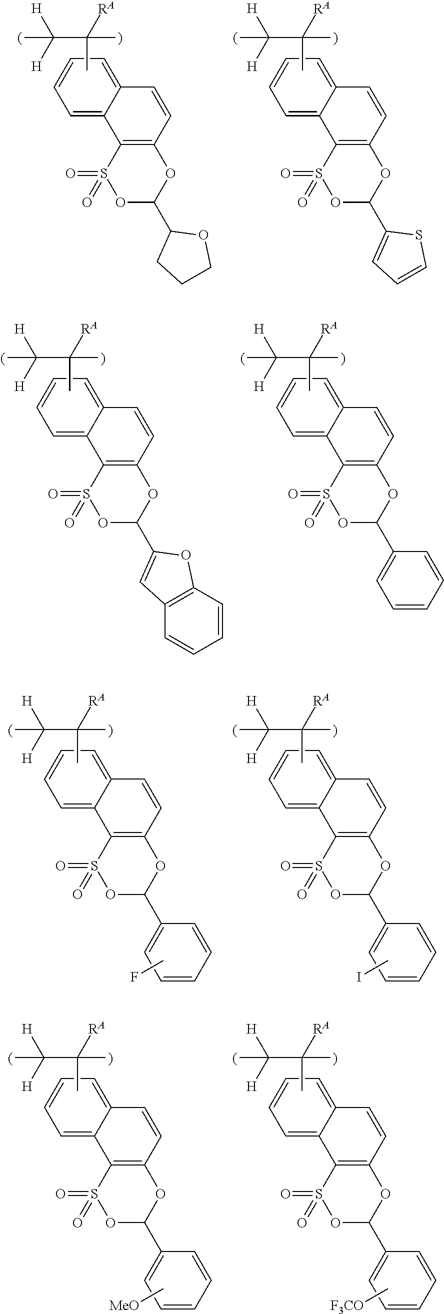

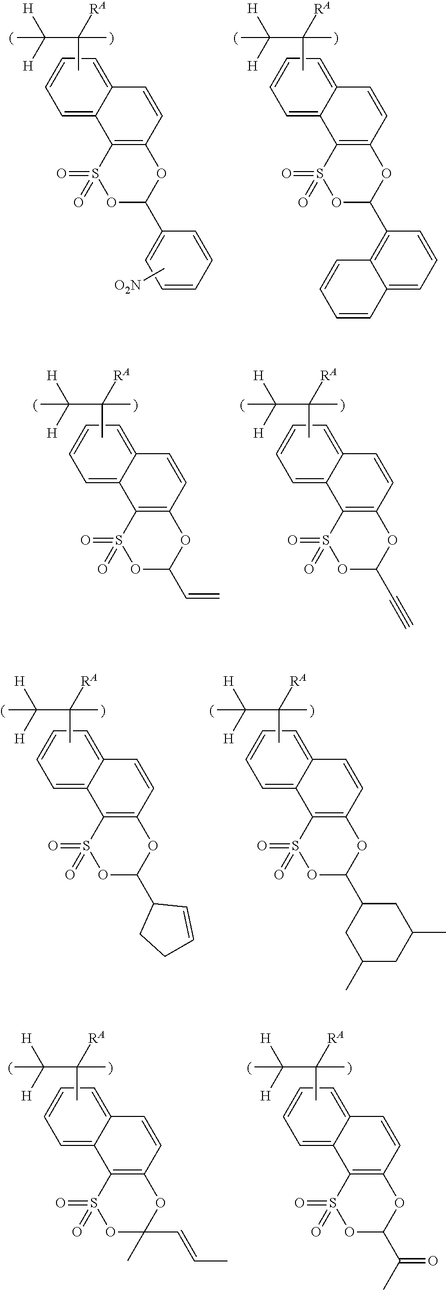

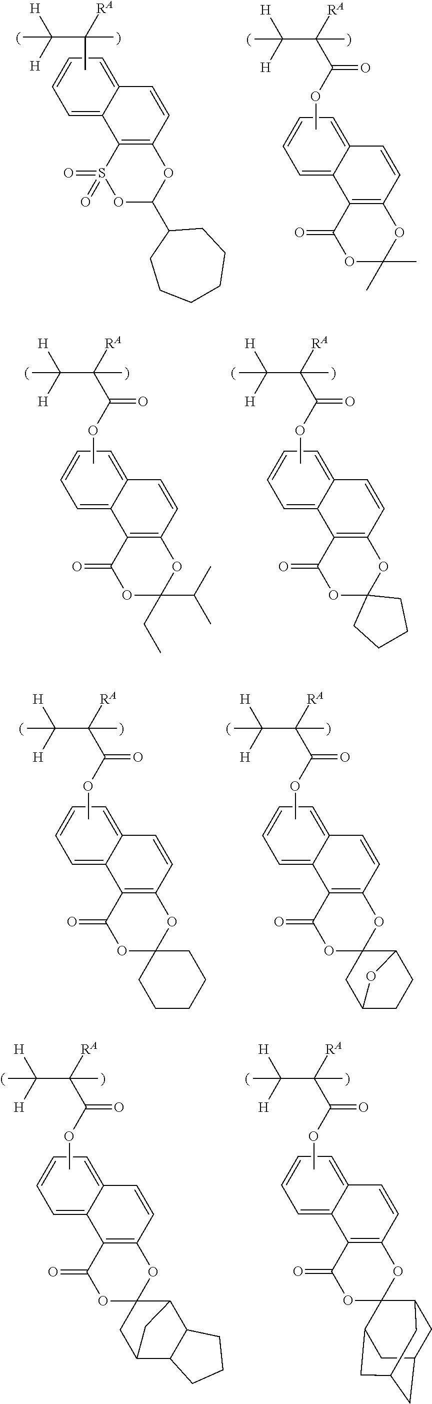

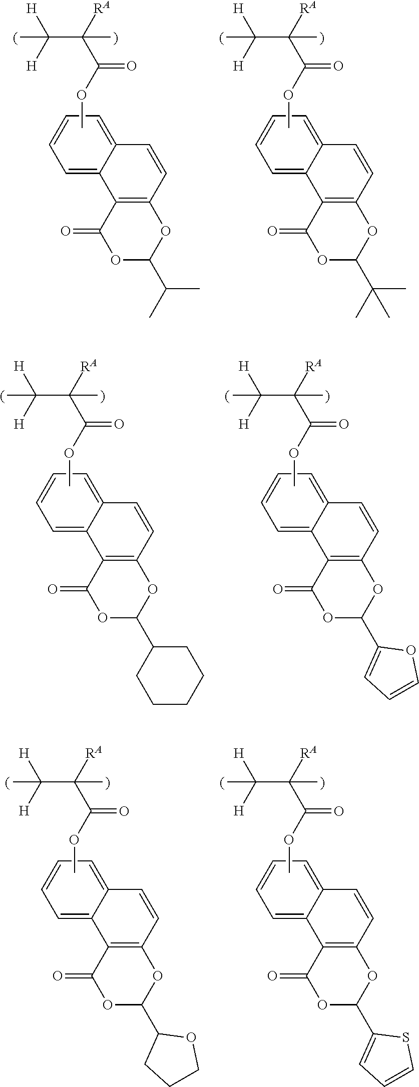

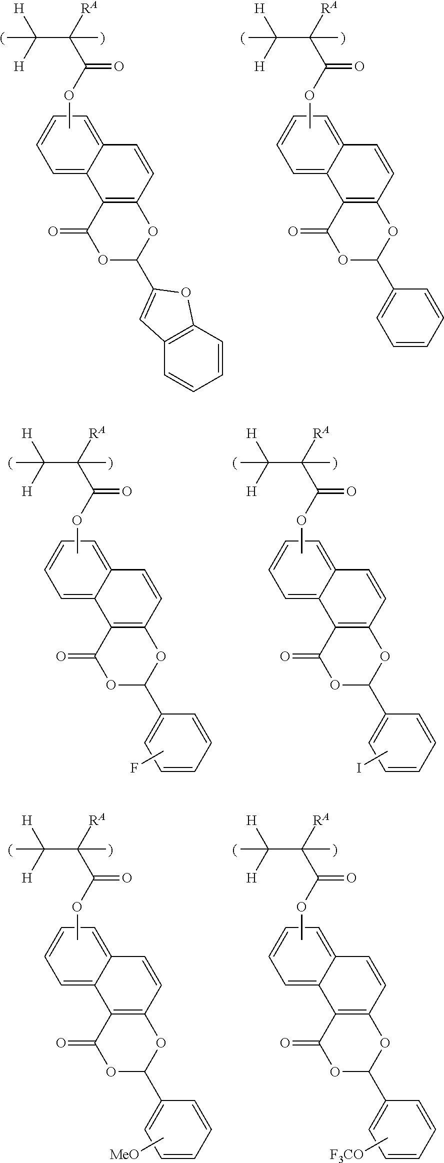

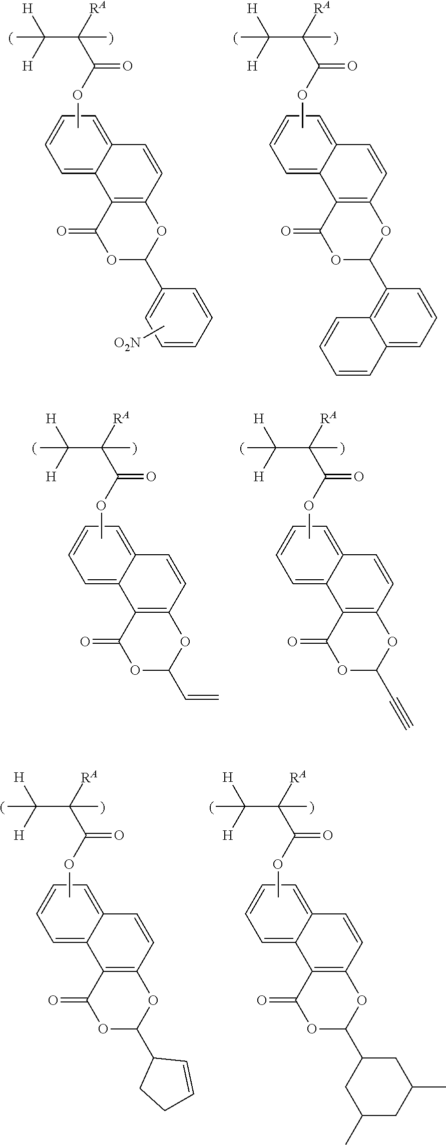

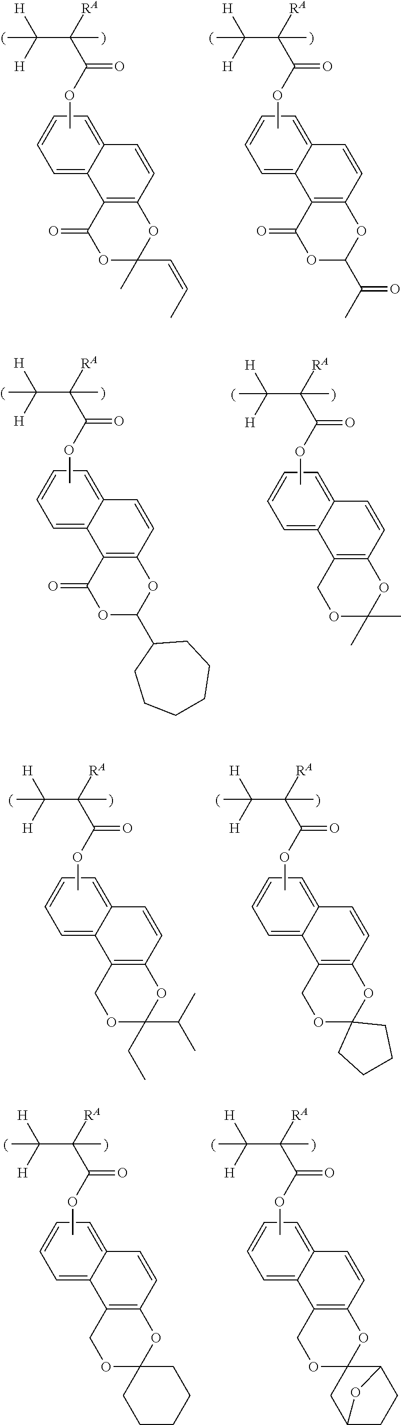

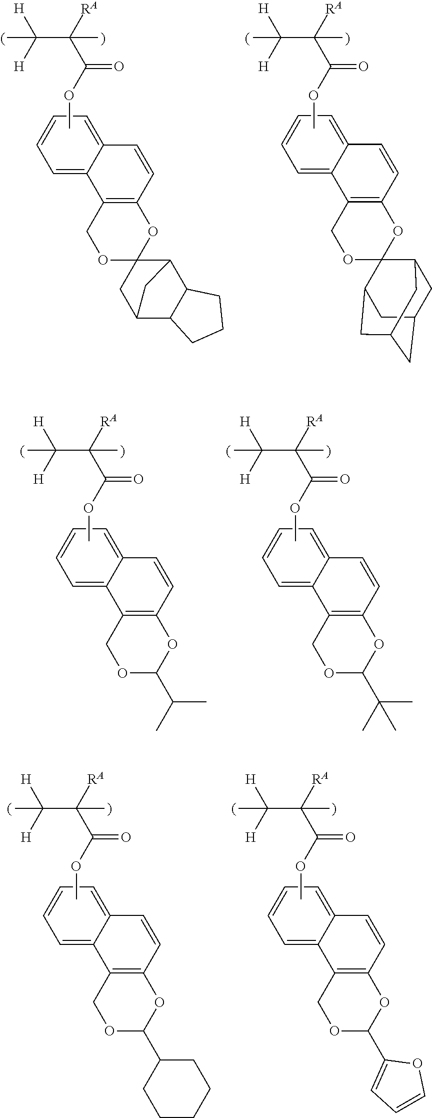

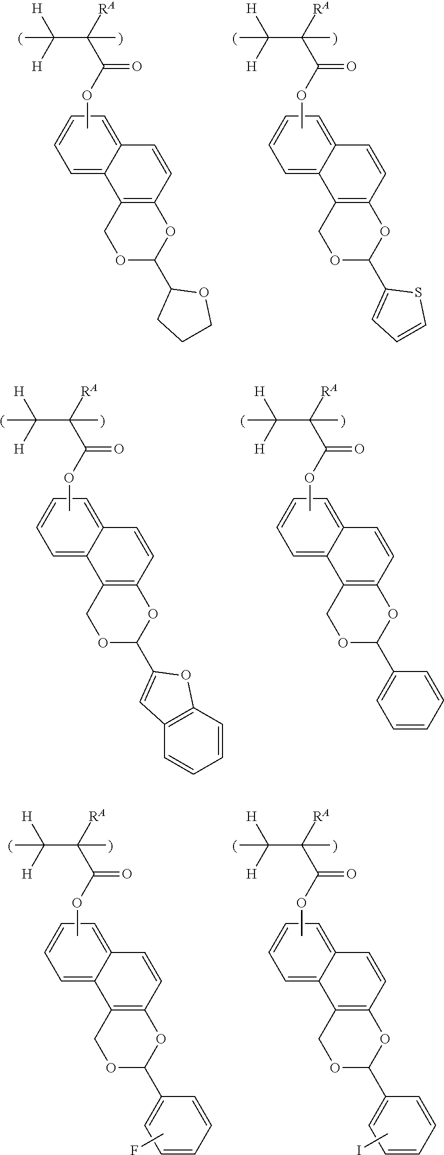

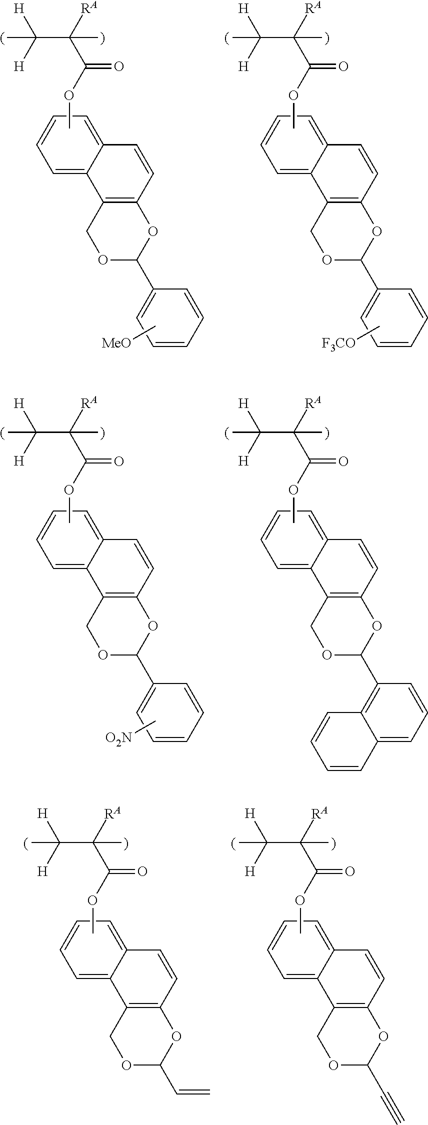

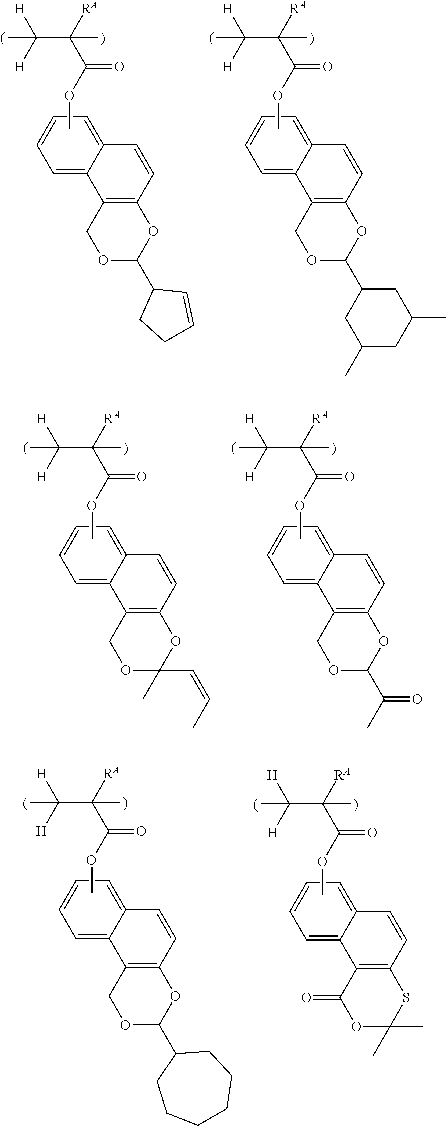

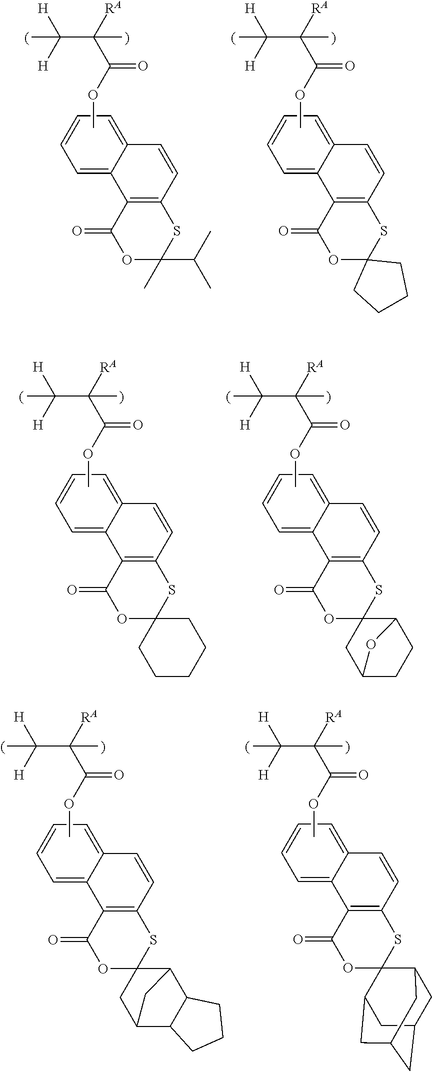

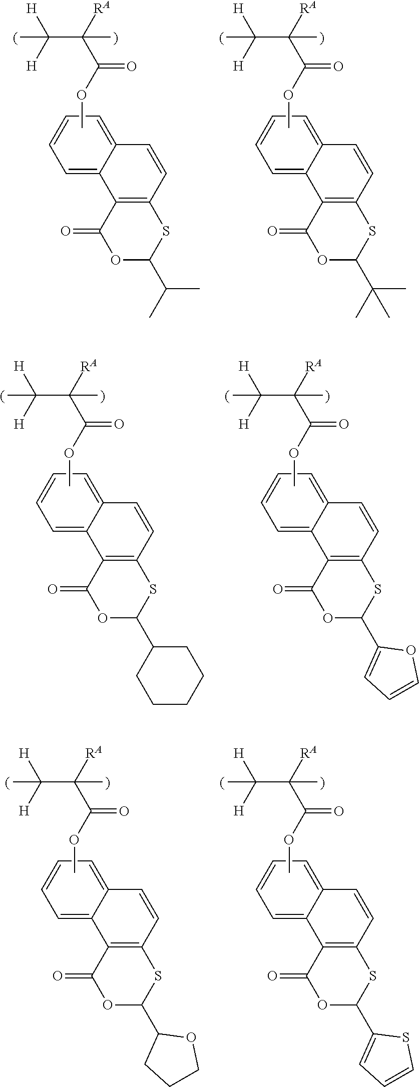

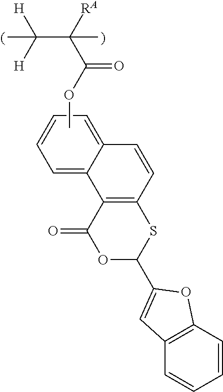

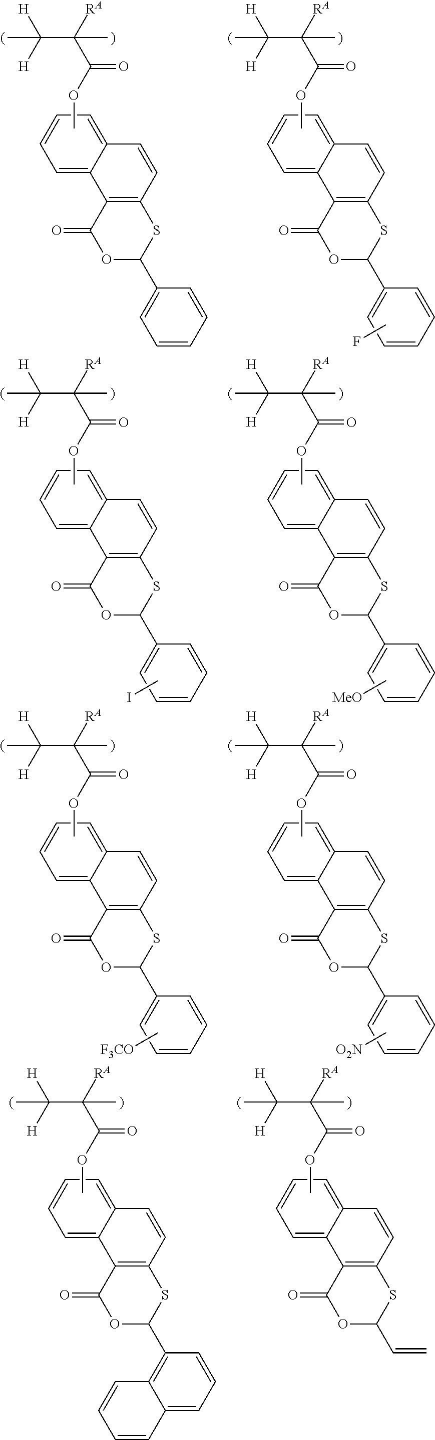

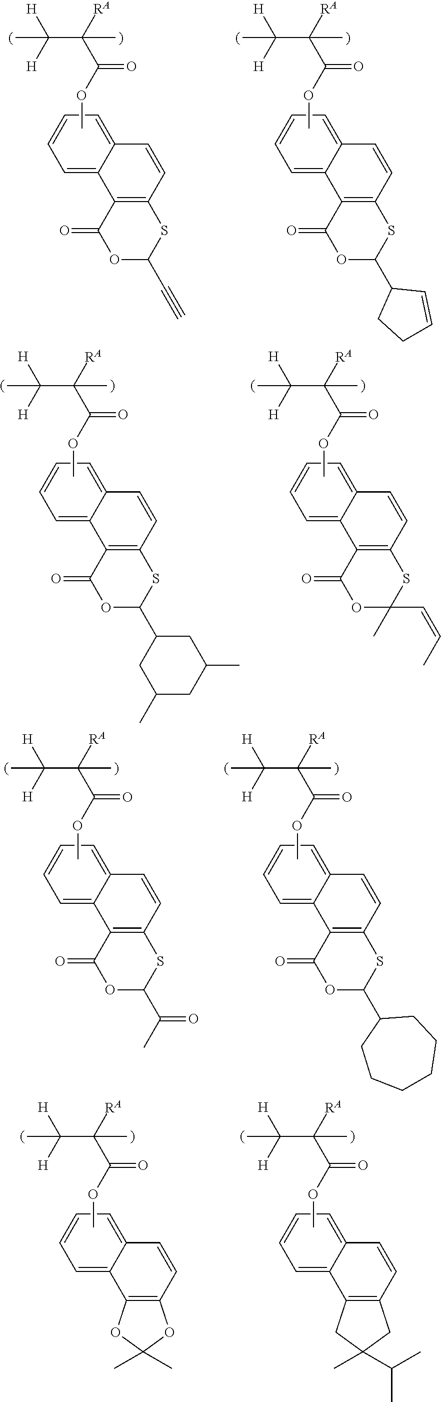

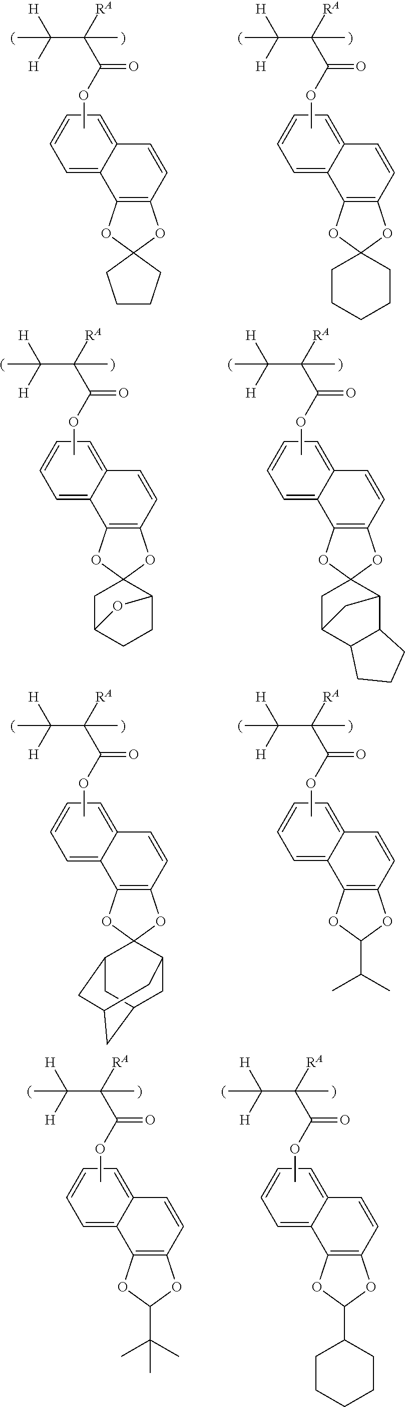

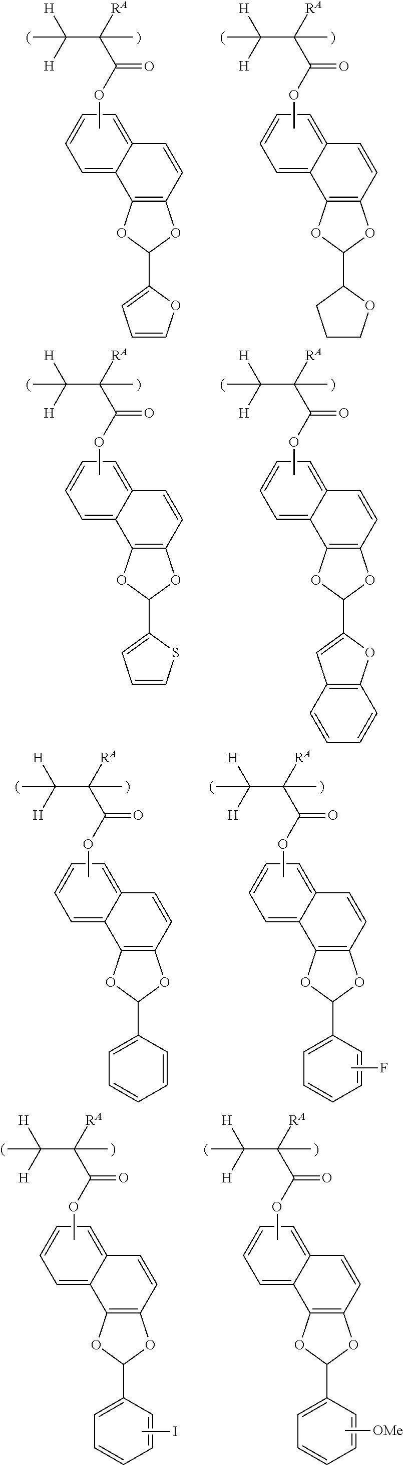

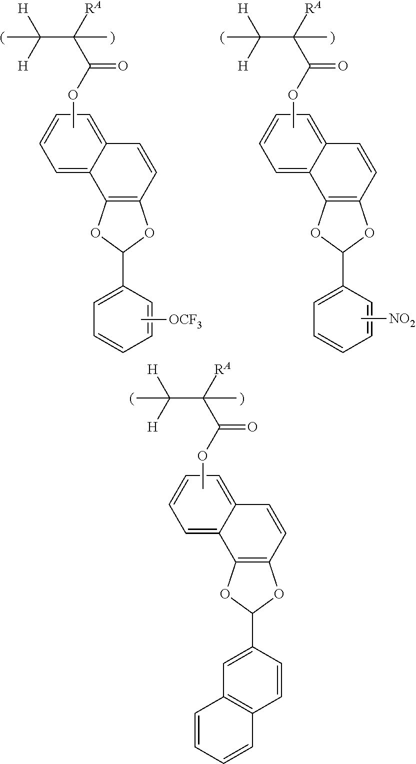

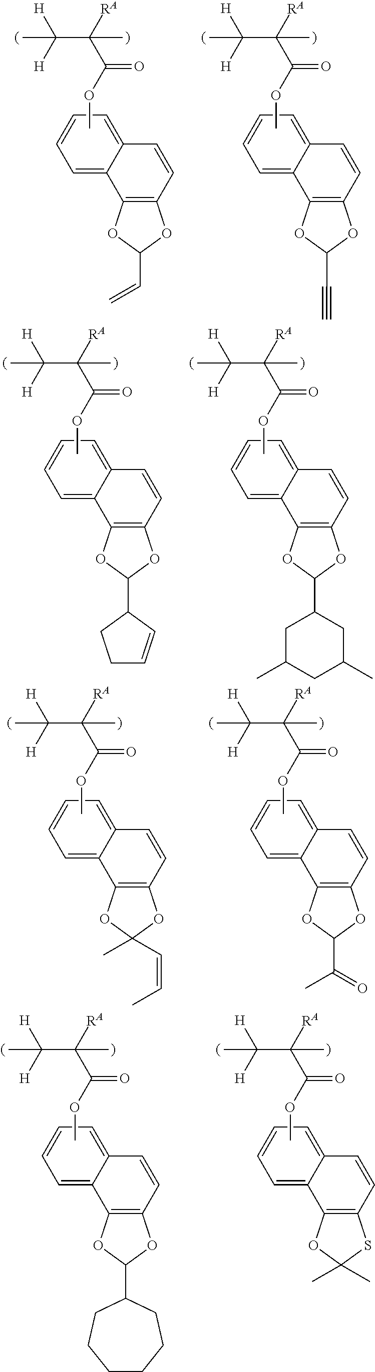

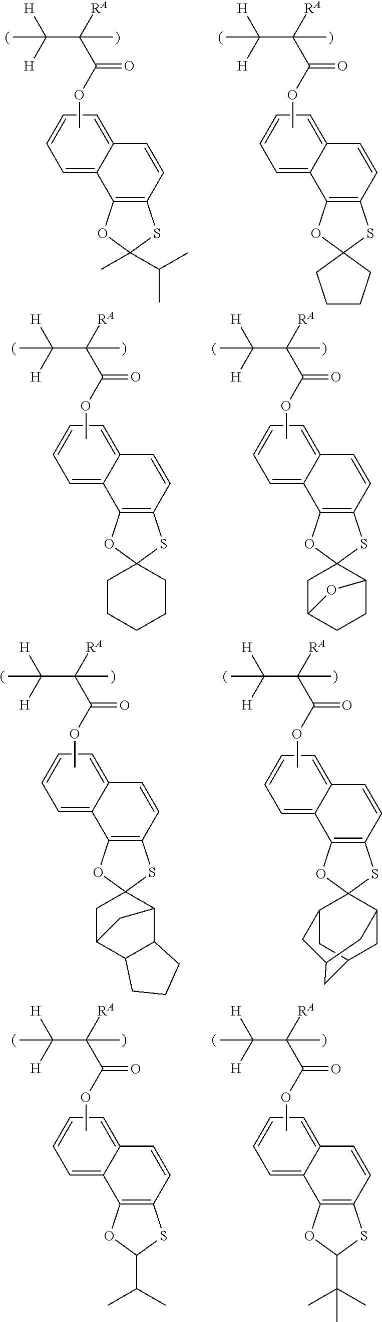

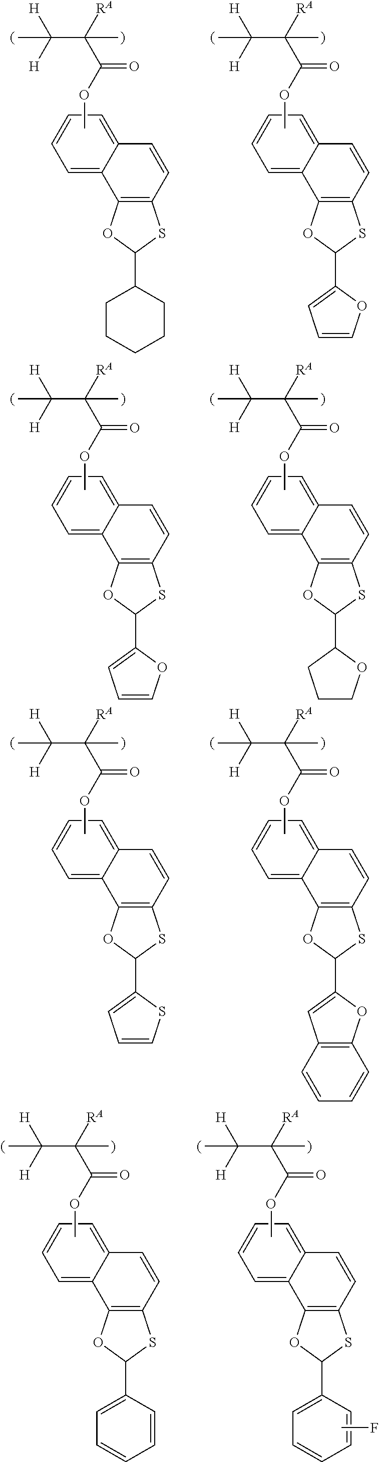

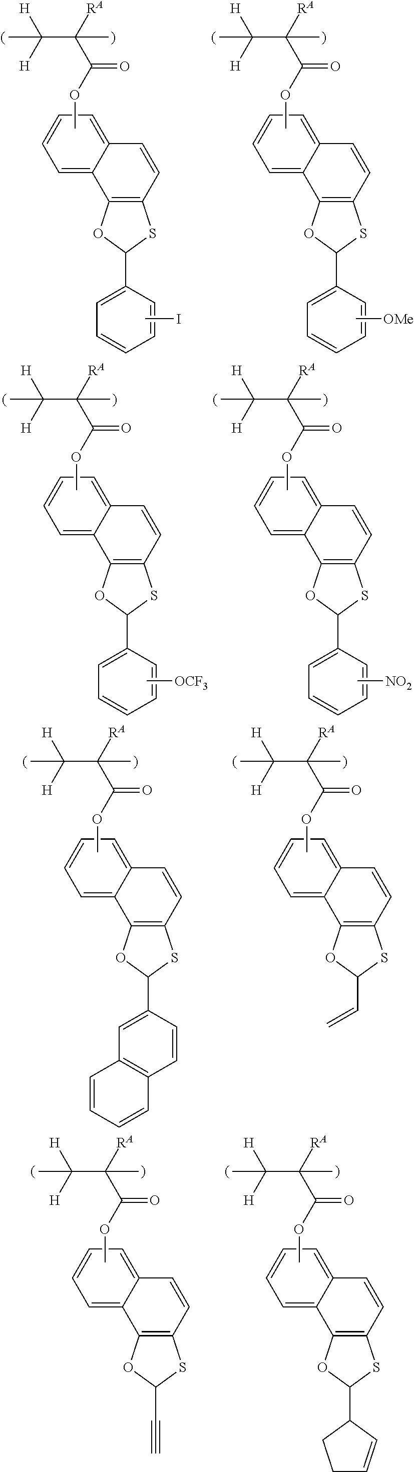

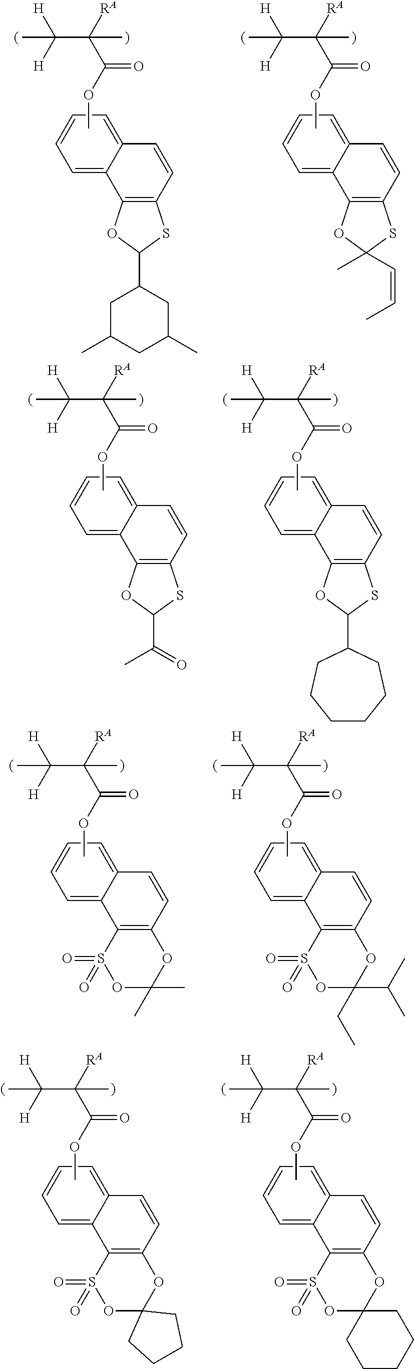

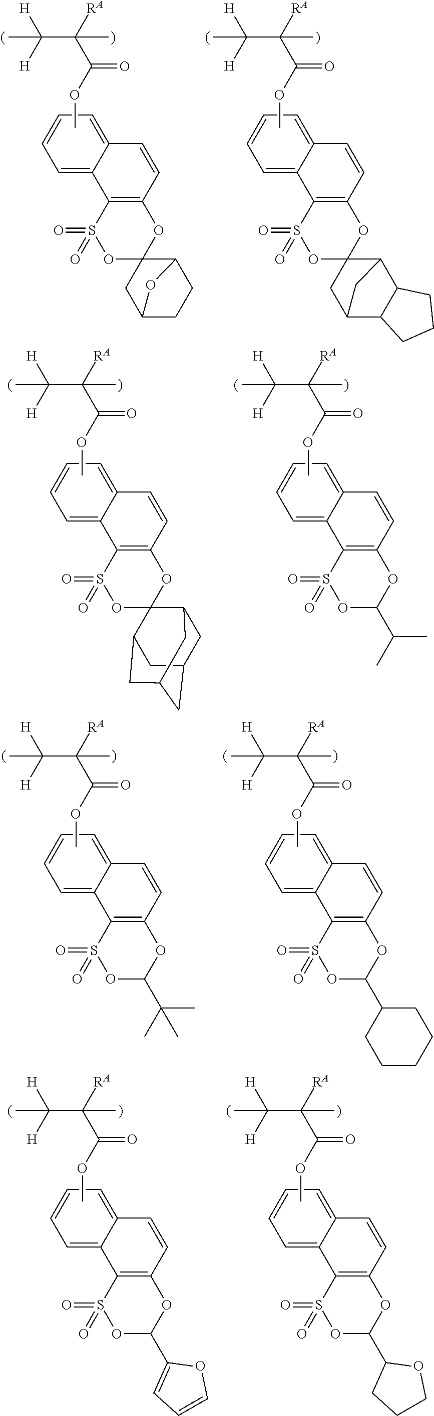

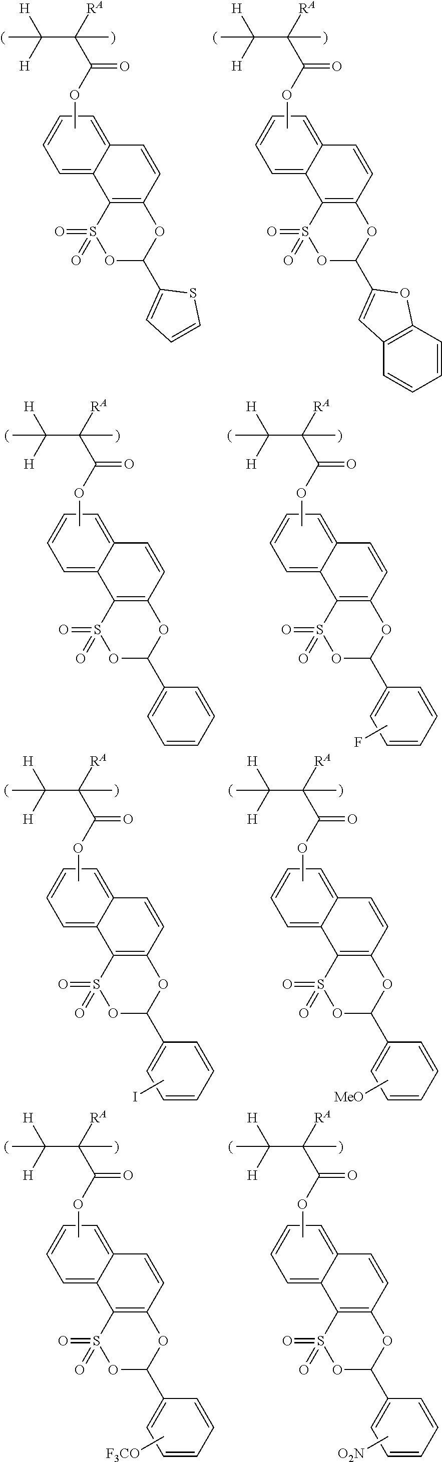

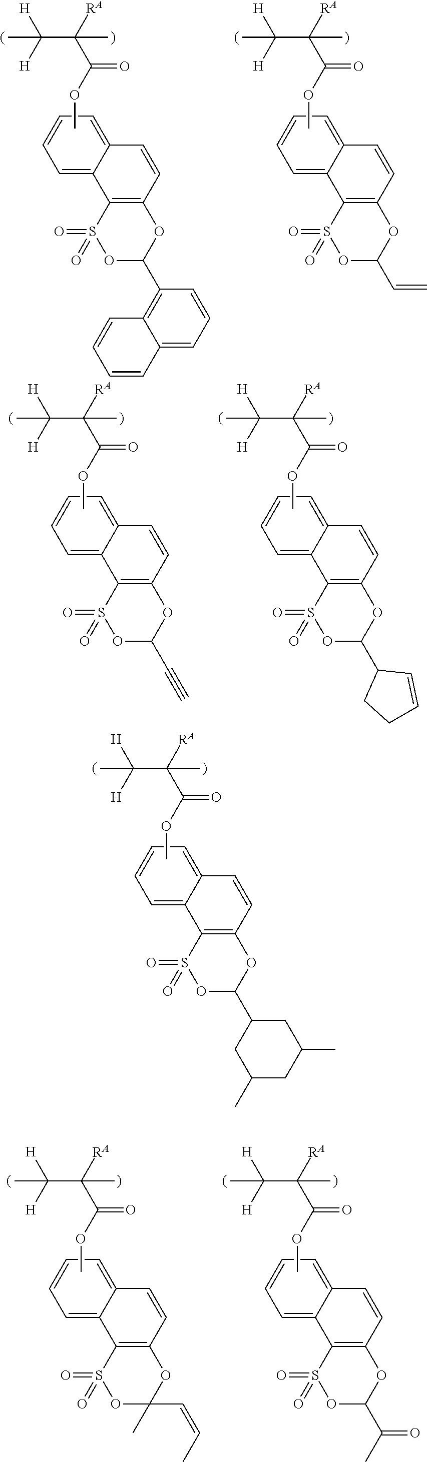

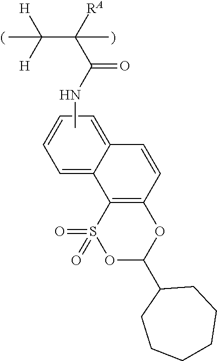

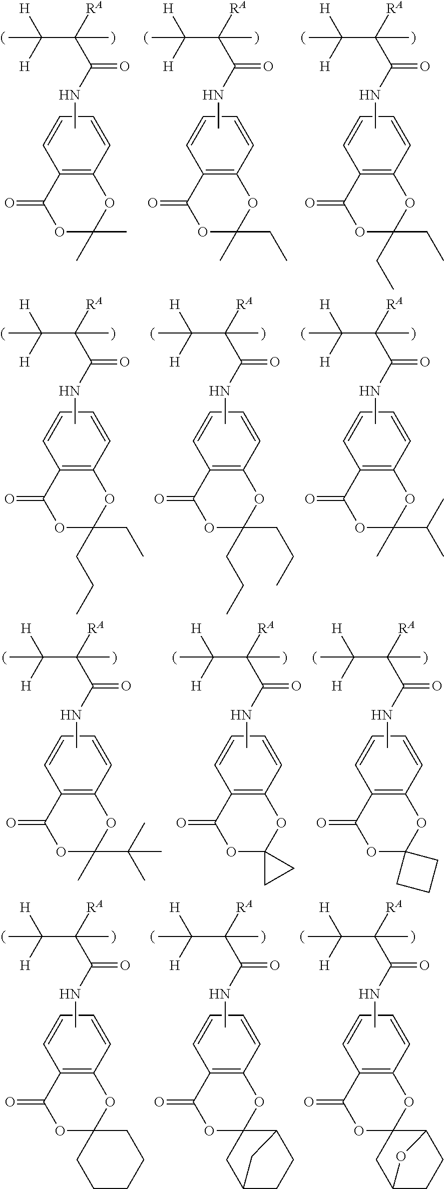

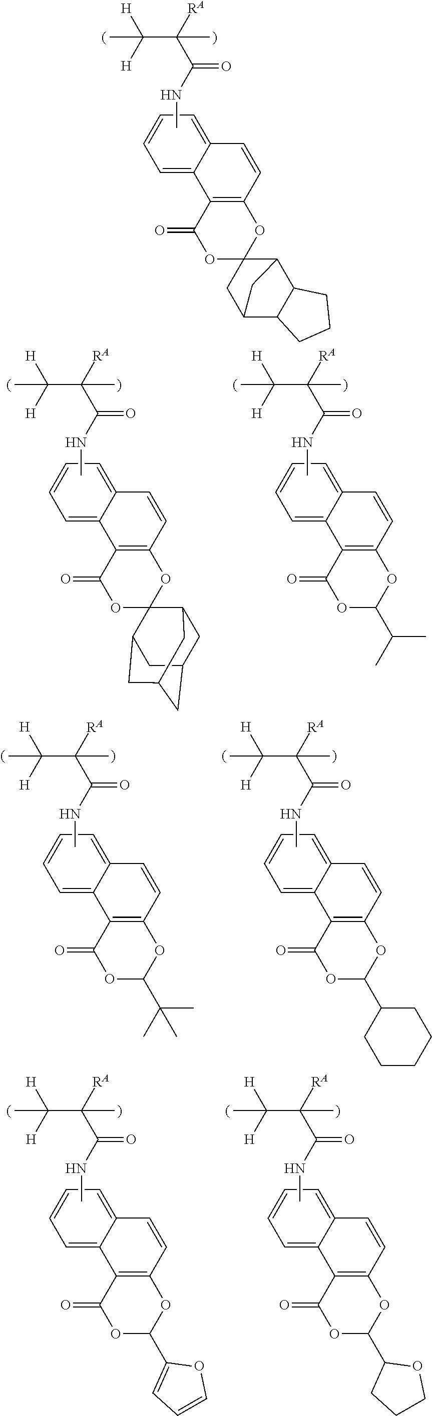

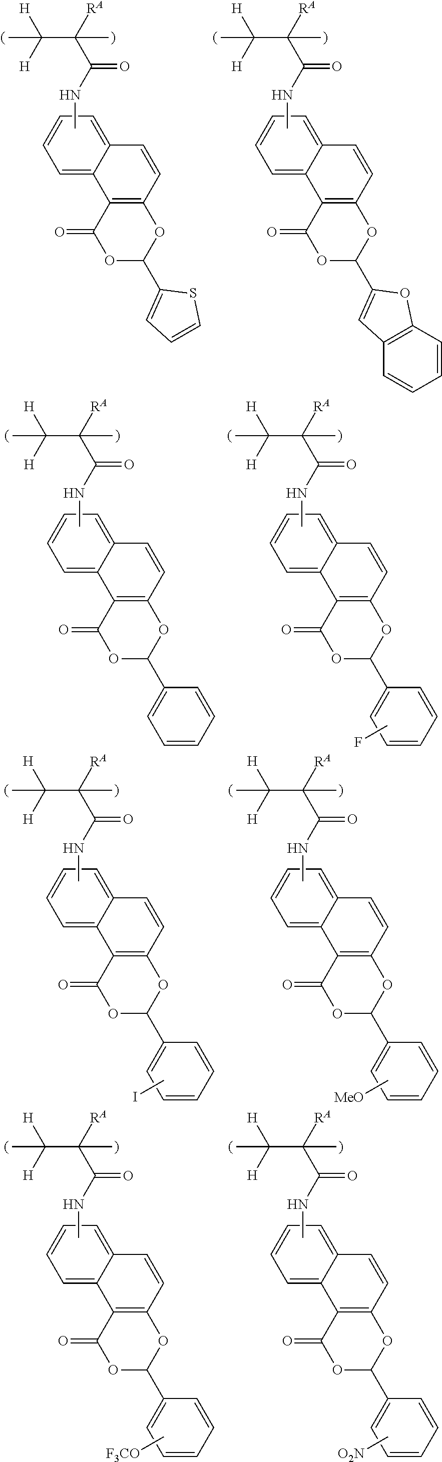

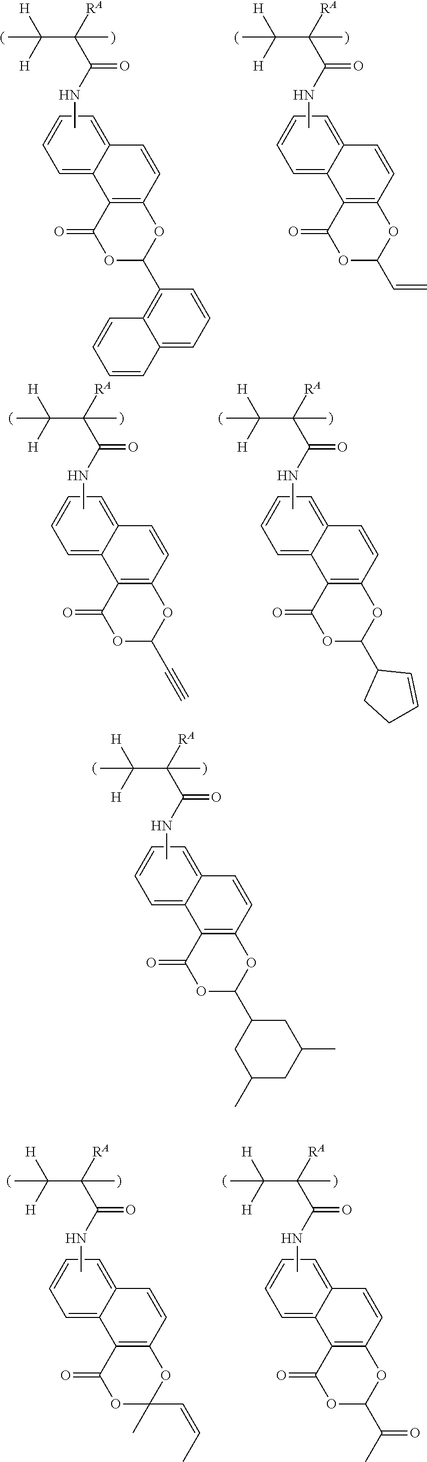

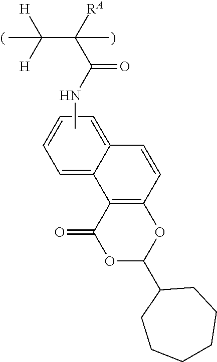

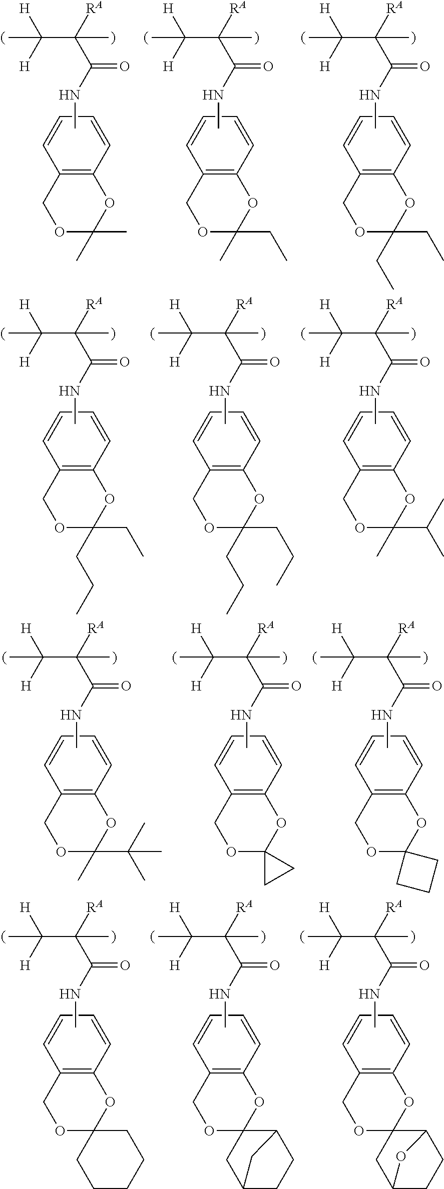

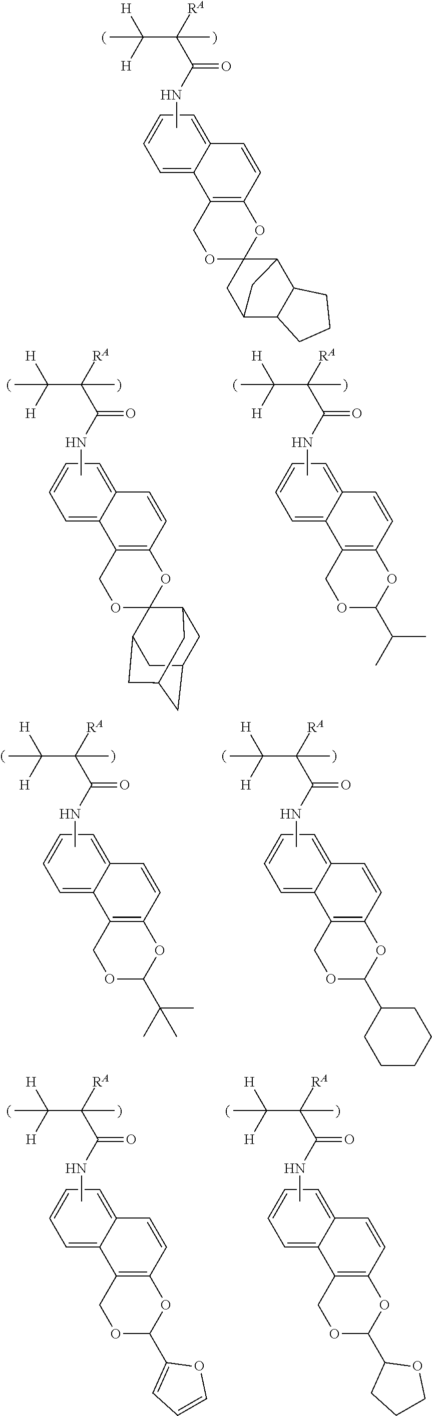

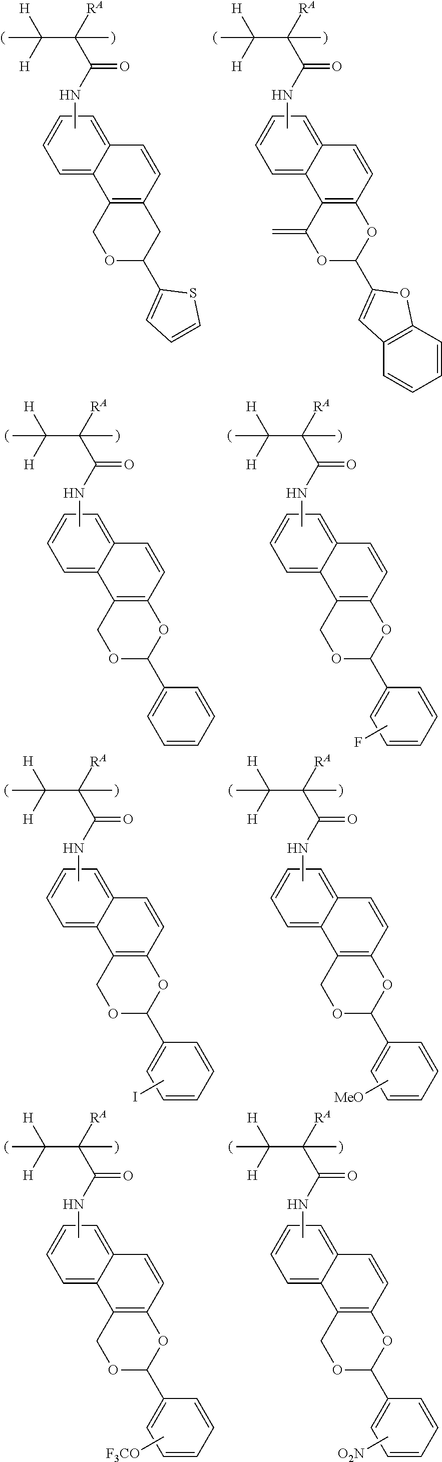

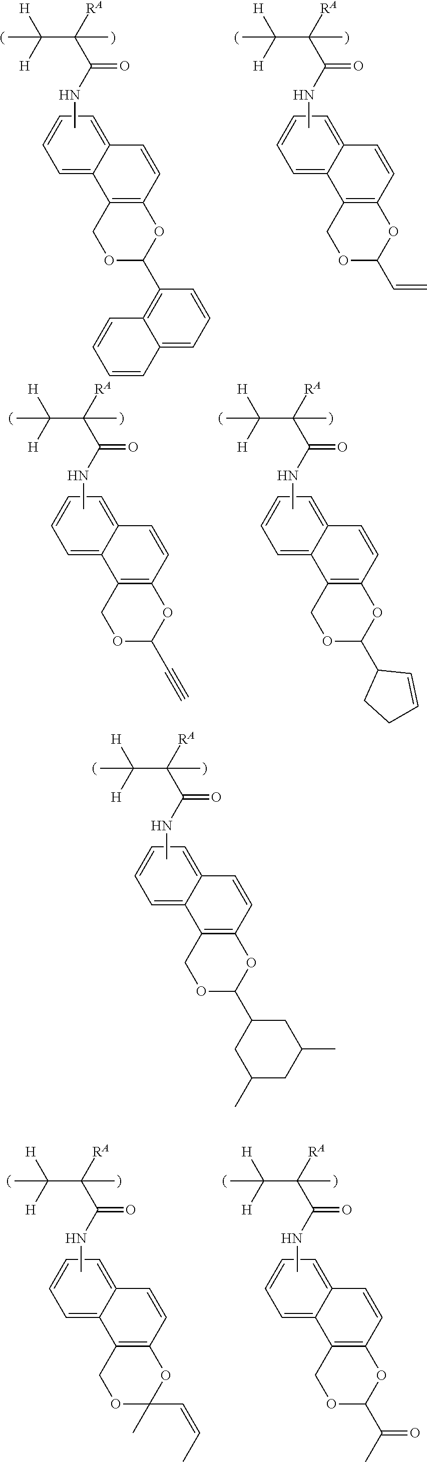

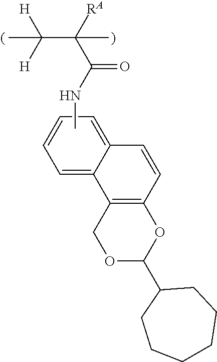

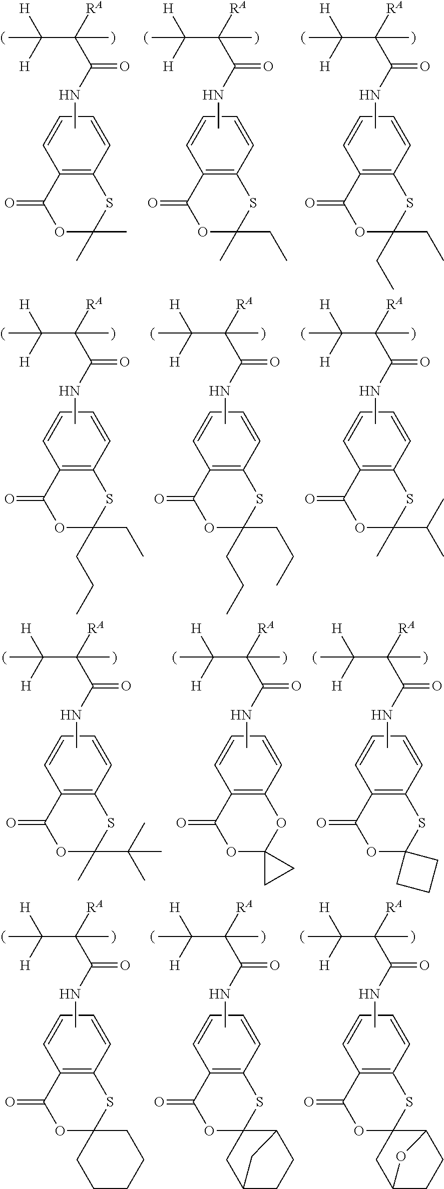

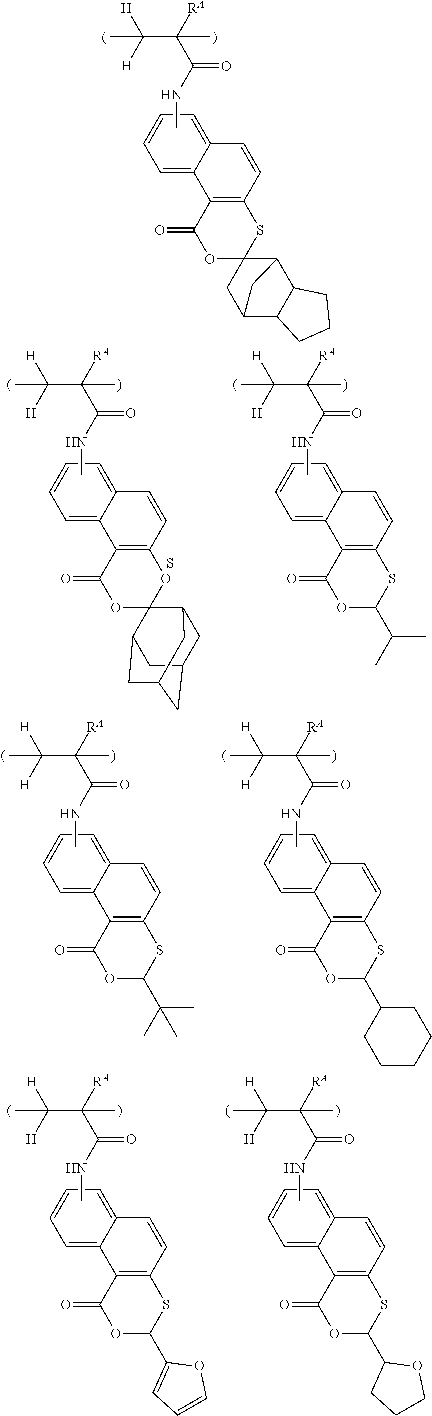

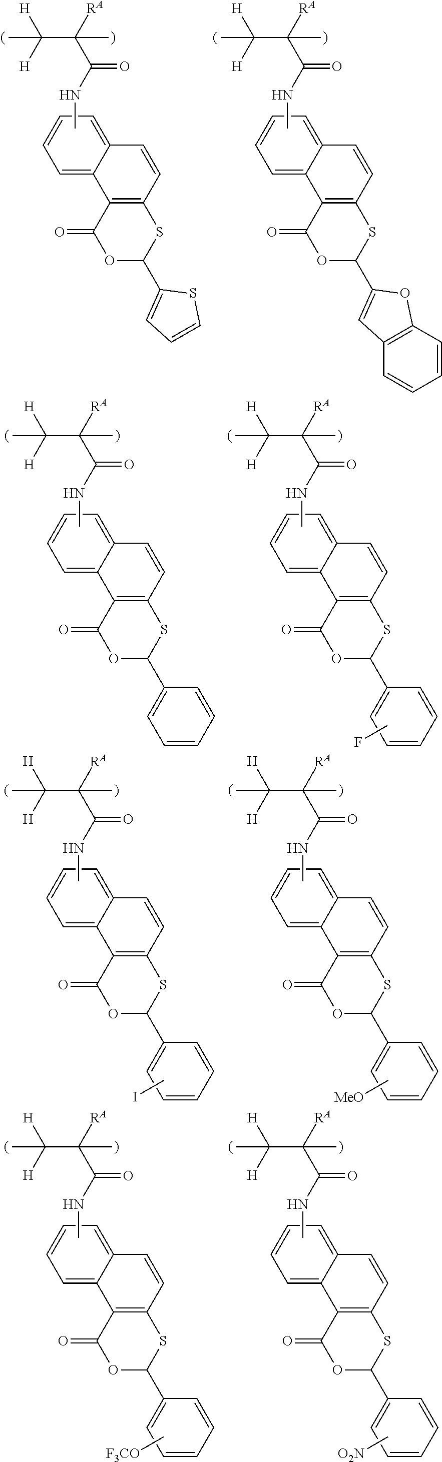

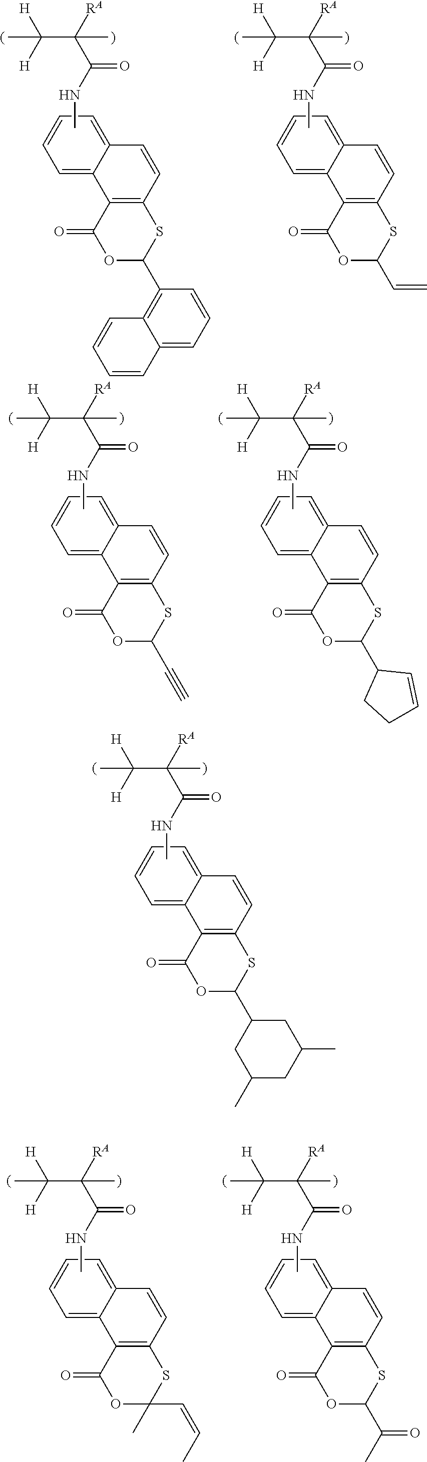

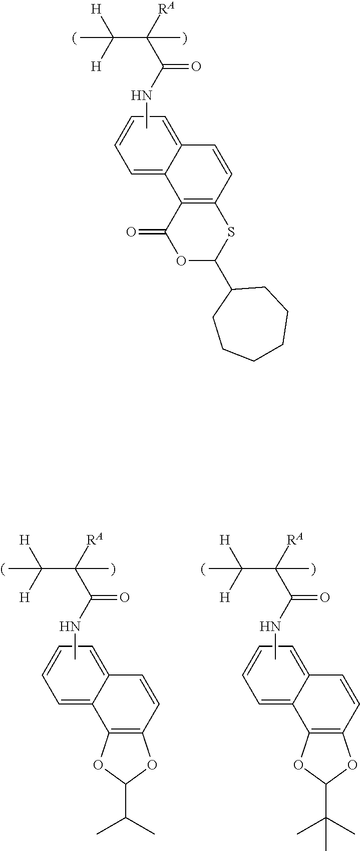

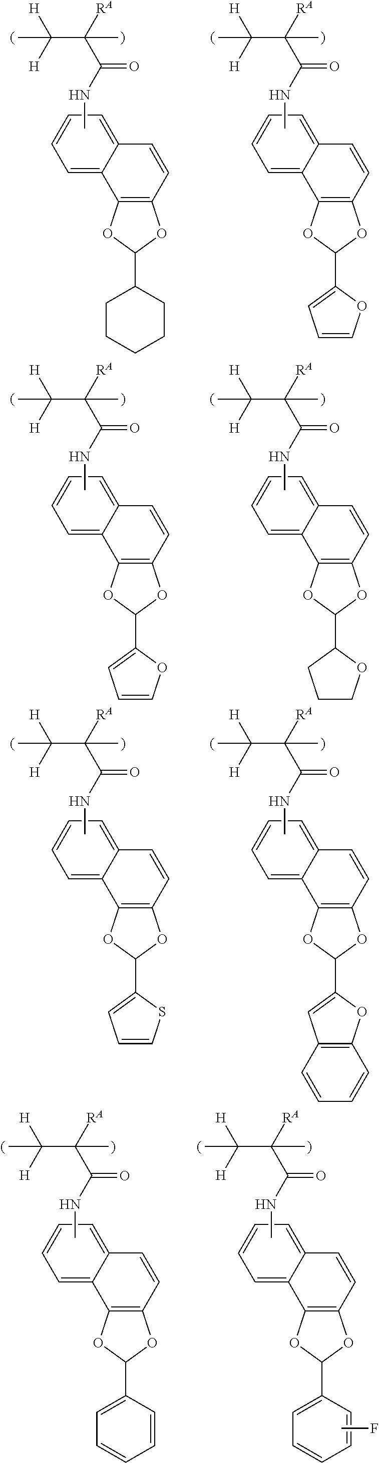

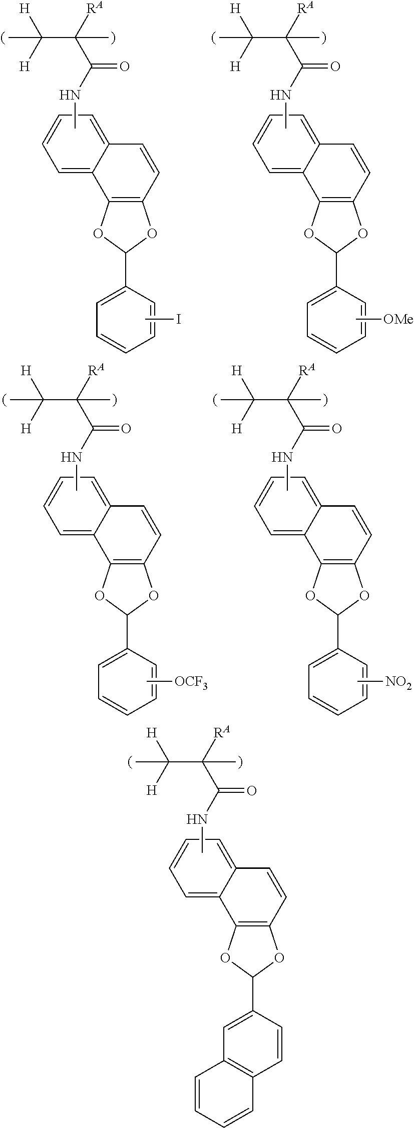

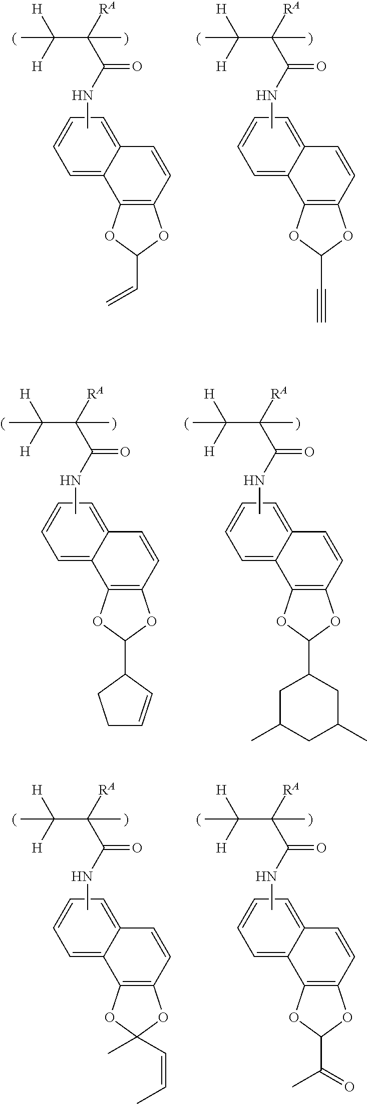

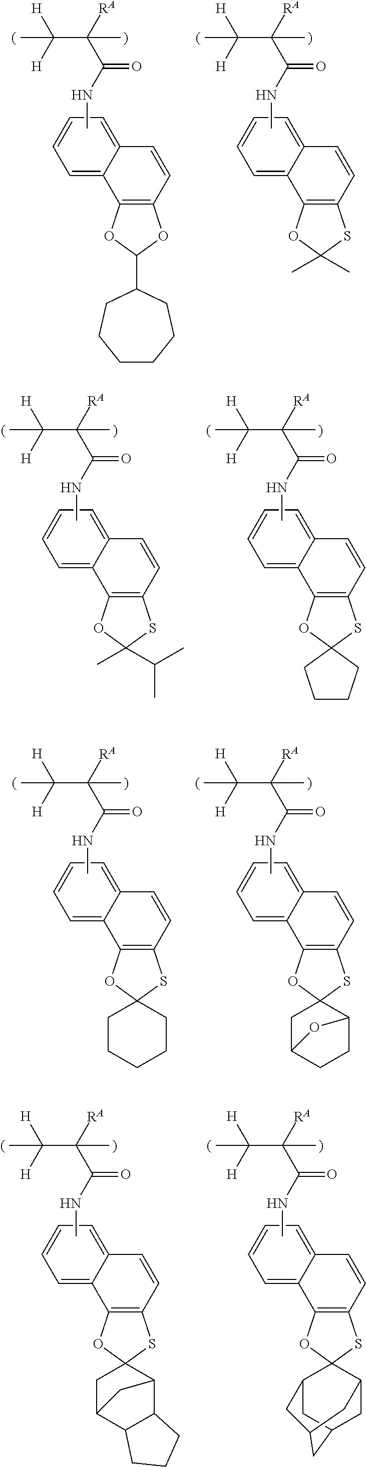

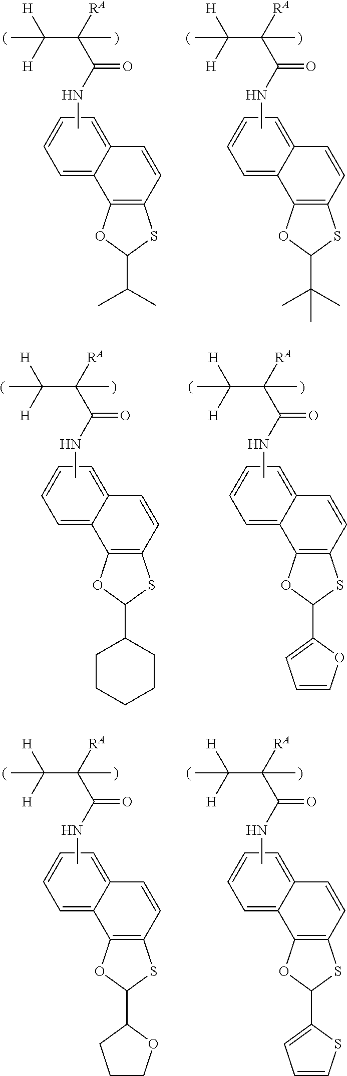

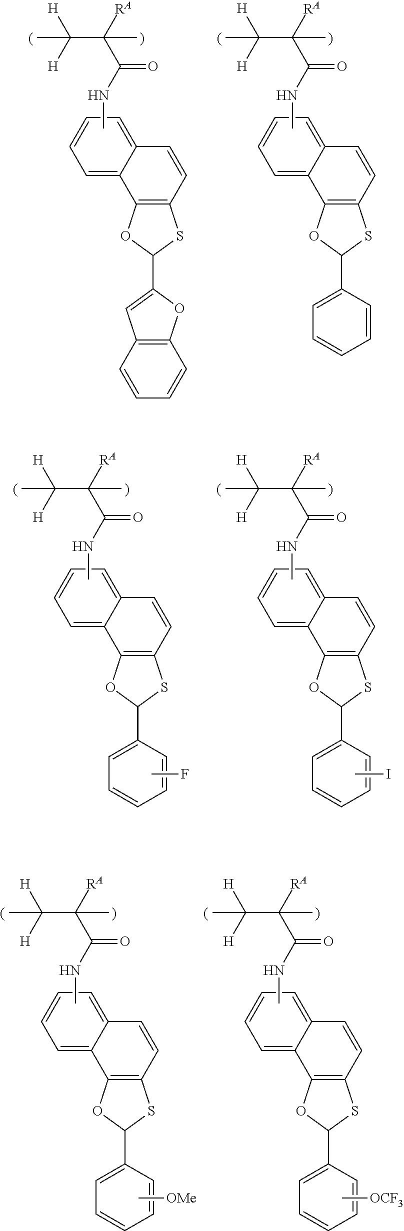

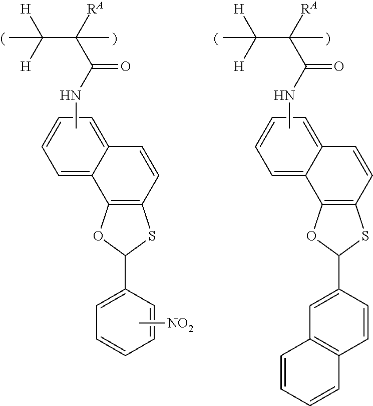

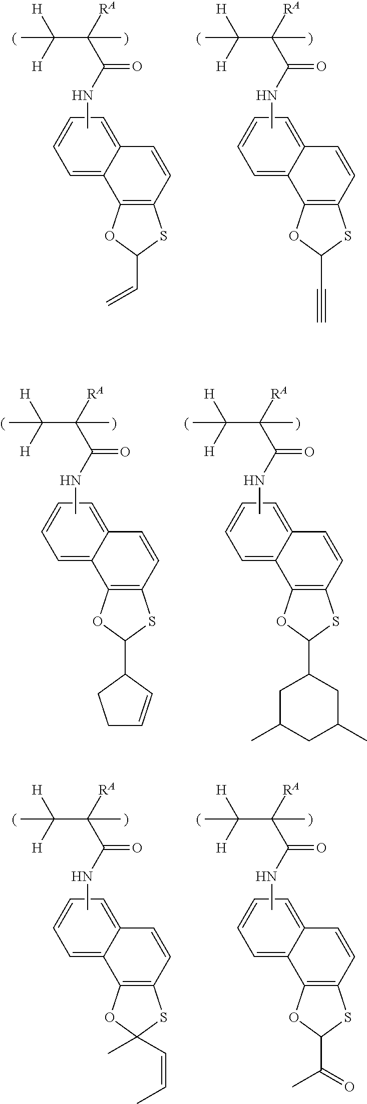

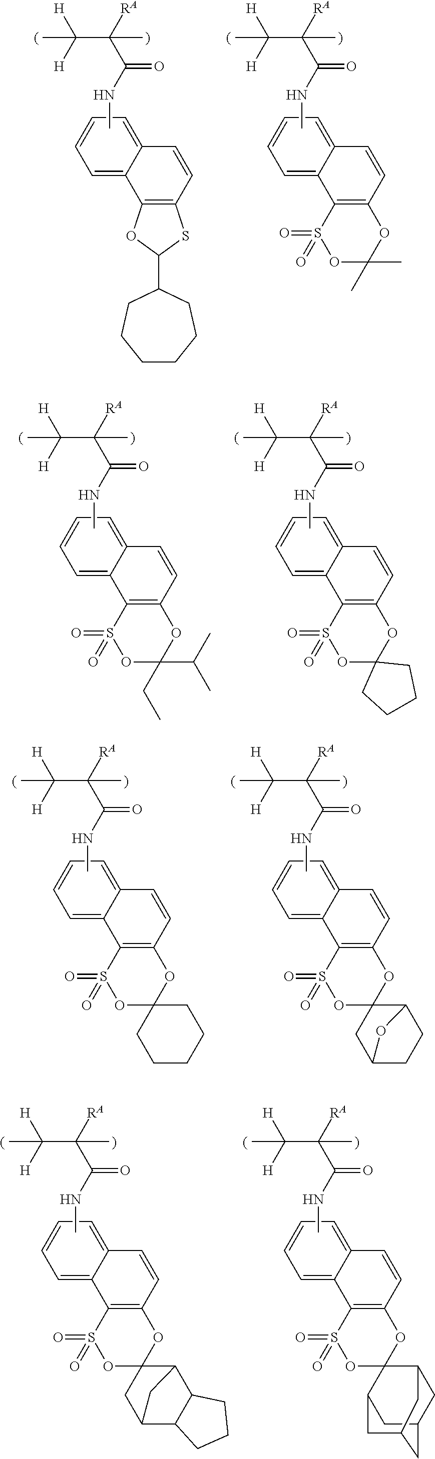

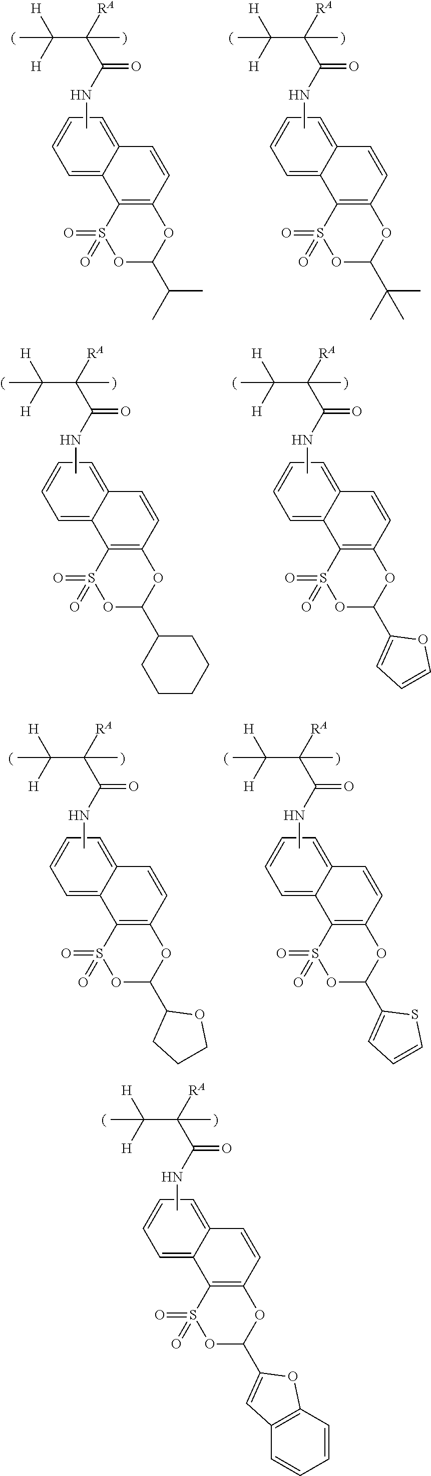

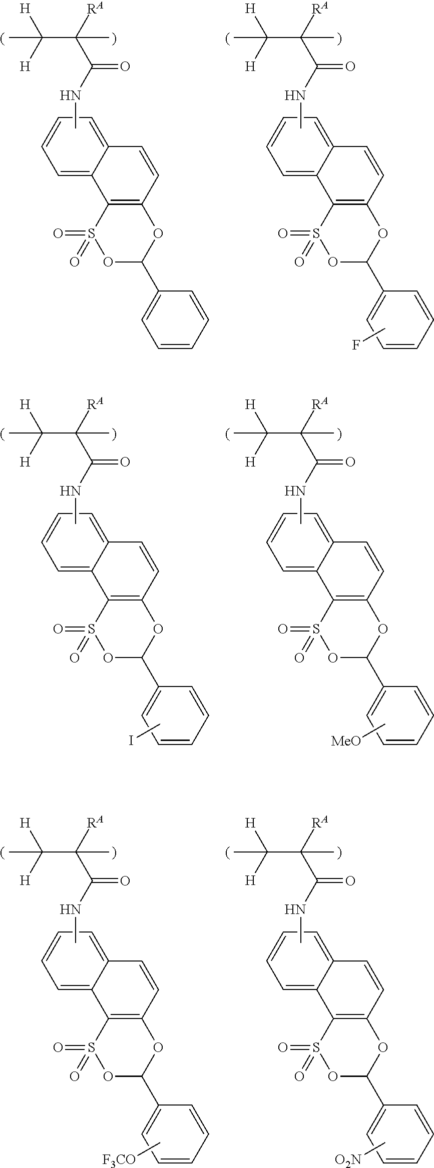

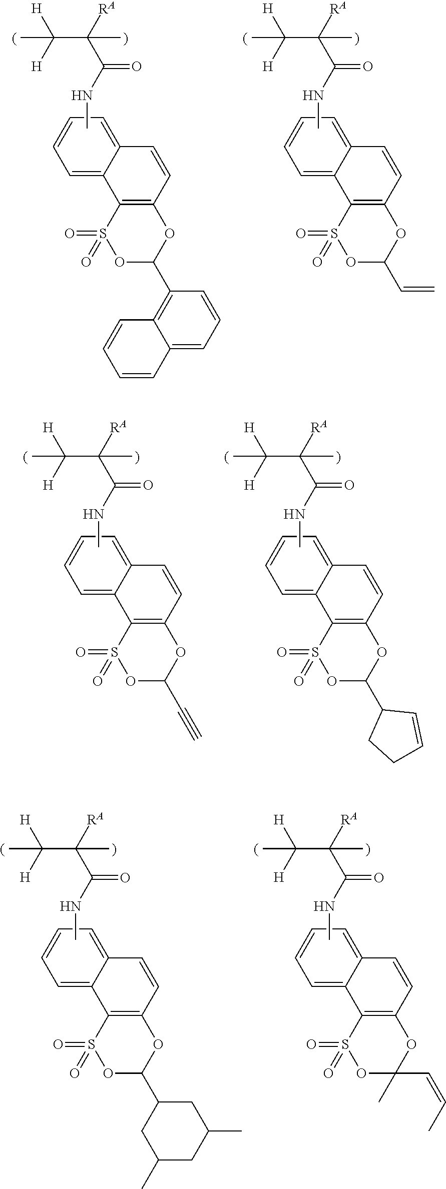

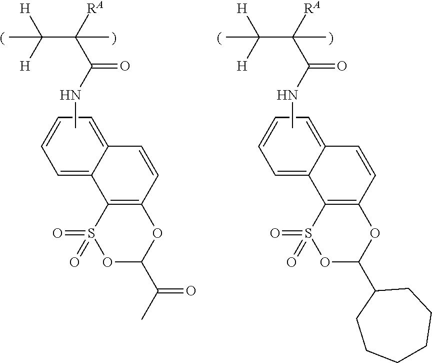

Preferably, the base polymer further comprises repeat units having the following formula (c) (hereinafter, also referred to as repeat units (c)).

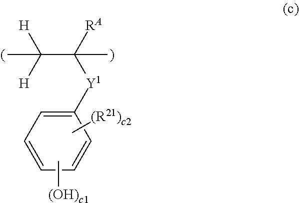

In formula (c), R^{A} is hydrogen, fluorine, methyl group, or trifluoromethyl group. Y¹ is a single bond, *—C(═O)—O— or *—C(═O)—NH—, * designates a point of attachment to the carbon atom in the backbone, R²¹ is halogen, nitro group, cyano group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, c1 is 1, 2, 3 or 4, and c2 is 0, 1, 2 or 3, The sum of c1+c2 is from 1 to 5.

Examples of repeat units (c) are shown below, but not limited thereto. Herein, R^{A} is as defined above.

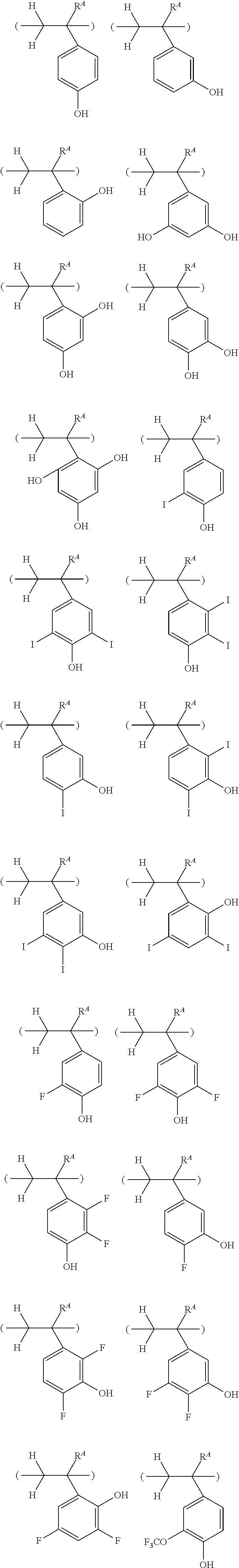

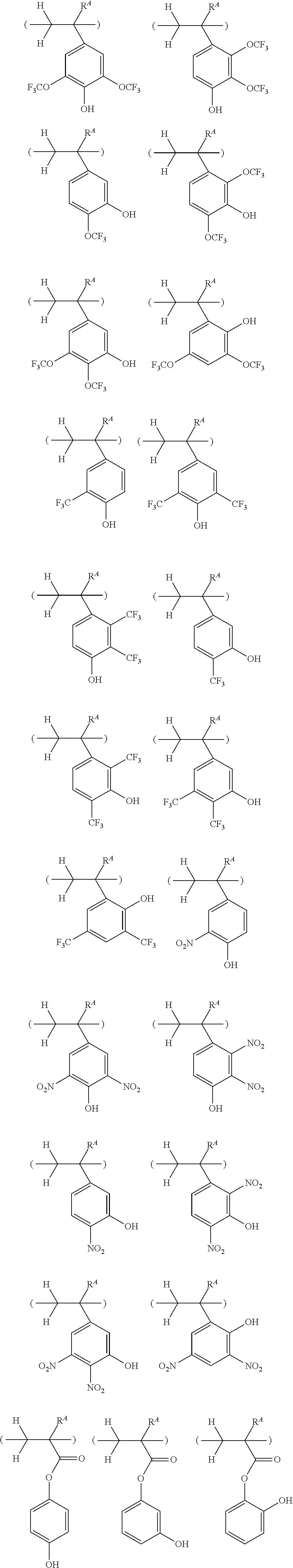

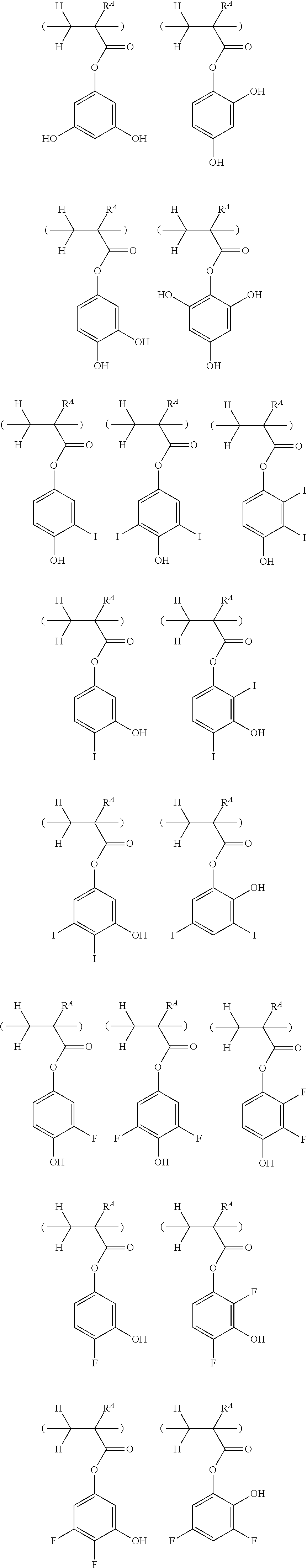

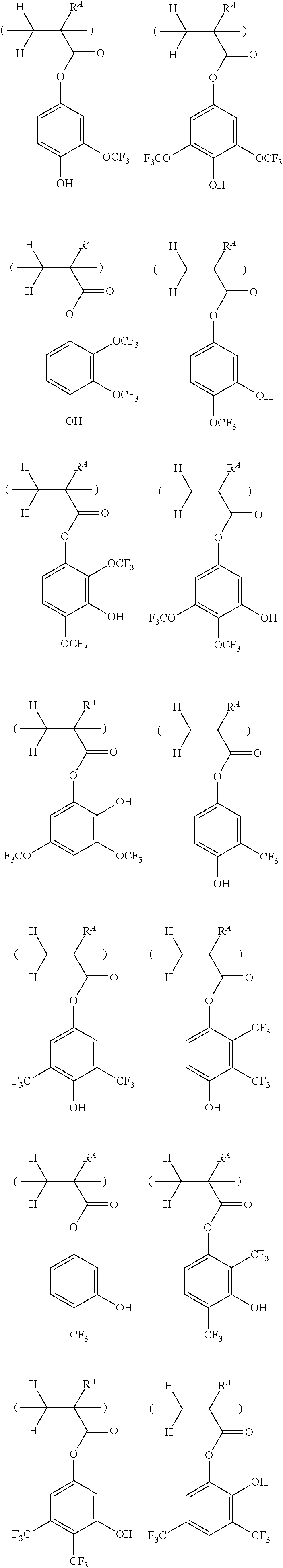

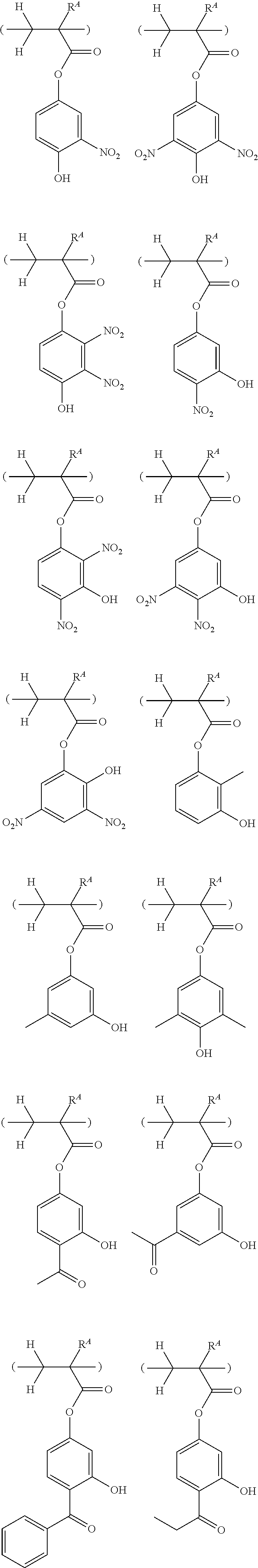

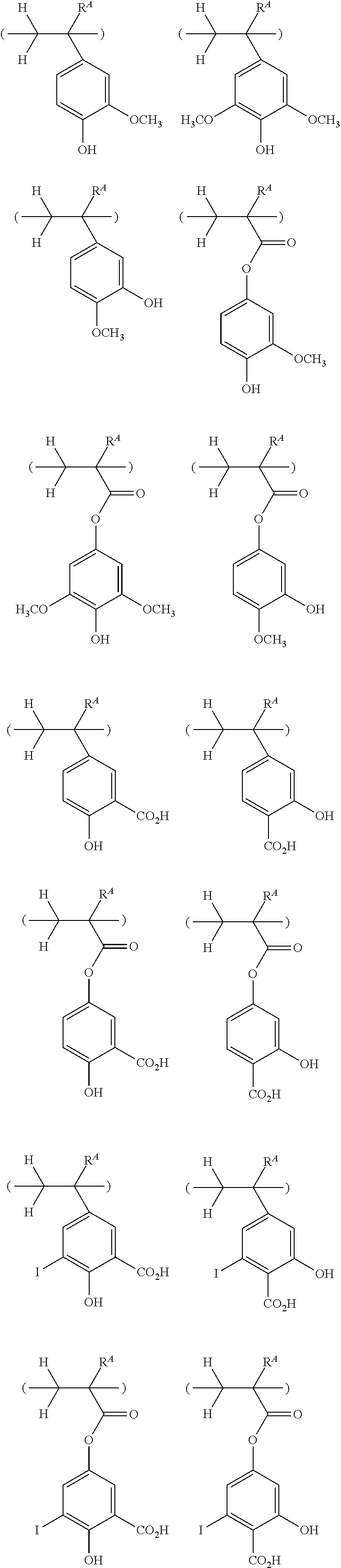

Preferably, the base polymer comprises repeat units derived from an onium salt monomer containing a fluorosulfonate anion having a polymerizable group and at least one iodine, and a sulfonium cation (hereinafter, also referred to as repeat units d).

Examples of the anion in repeat units (d) include structures described in JP-A 6973274, paragraphs [0023]-[0029], JP 2023-172928, paragraphs [0032]-[0038], and JP-A 2024-043941, paragraphs [0032]-[0047].

As examples of the anion in repeat units d, structures having the formula (d1) are also preferred.

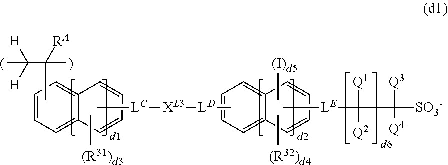

In formula (d1), d1 is 0 or 1. The sulfonium salt monomer has a benzene ring when d1 is 0, and a naphthalene ring when d1 is 1, and d1 is preferably 0 from the aspect of solvent solubility. The subscript d2 is 0 or 1. The sulfonium salt monomer has a benzene ring when d2 is 0, and a naphthalene ring when d2 is 1, and d2 is preferably 0 from the aspect of solvent solubility. The subscript d3 is 0, 1, 2, 3 or 4. From the aspect of reactant availability, d3 is preferably 0, 1 or 2, more preferably 0 or 1. The subscript d4 is 0, 1, 2, 3 or 4, preferably 0, 1, 2 or 3, more preferably 0, 1 or 2, still more preferably 0 or 1. The subscript d5 is 1, 2, 3, 4, 5 or 6. As the number of iodine atoms in the anionic structure becomes larger, the amount of absorption of EUV increases, but precipitation in the resist composition may occur due to reduced solvent solubility. Therefore, d5 is preferably 1, 2 or 3, more preferably 1 or 2. The sum of d4+d5 is from 1 to 4 when d2 is 0, and the sum of d4+d5 is from 1 to 6 when d2 is 1. The subscript d6 is 0, 1, 2, 3 or 4, preferably 0, 1, 2 or 3, more preferably 1.

In formula (d1), R^{A} is hydrogen, fluorine, methyl group or trifluoromethyl group. Of these, hydrogen atom and methyl group are preferred, and hydrogen is more preferred.

In formula (d1), the iodine atom in the aromatic ring of the anion is preferably bound in an ortho position with respect to a carbon atom to which L^{B} is attached. Since an iodine atom is an element having a large atomic radius, the rotation of the bond axis between the aromatic ring to which a polymerizable group is attached and the aromatic ring to which an iodine atom is attached is suppressed, leading to improved rigidity of the polymer.

In formula (d1), R³¹ is halogen, or a C₁-C₂₀ hydrocarbyl group which may contain a heteroatom. The halogen is preferably fluorine, chlorine, bromine or iodine, more preferably fluorine or iodine. The hydrocarbyl group may be saturated or unsaturated, and may be straight, branched, or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl and icocyl groups; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl and adamantyl groups; C₂-C₂₀ alkenyl groups such as vinyl, allyl, propenyl, butenyl and hexenyl groups; C₃-C₂₀ cyclic unsaturated hydrocarbyl groups such as a cyclohexenyl group; C₂-C₂₀ aryl groups such as phenyl and naphthyl groups; C₇-C₂₀ aralkyl groups such as benzyl, 1-phenylethyl and 2-phenylethyl groups; and combinations thereof. Some or all of hydrogen atoms of the hydrocarbyl group may be replaced by a group containing a heteroatom such as oxygen, sulfur, nitrogen or halogen, some constituent —CH₂— of the hydrocarbyl group may be replaced by a group containing a heteroatom such as oxygen, sulfur or nitrogen, and as a result, the hydrocarbyl group may contain a hydroxy group, a cyano group, fluorine, chlorine, bromine, iodine, a carbonyl group, an ether bond, an ester bond, a sulfonic ester bond, a carbonate bond, a lactone ring, a sultone ring, carboxylic anhydride (—C(═O)—O—C(═O)—), a haloalkyl group, or the like. A plurality of R³¹ may be identical or different when d3 is 2, 3 or 4.

A plurality of R³¹ moieties may bond together to form a ring with the aromatic ring carbon atom to which they are attached when d3 is 2, 3 or 4. Examples of the ring formed herein include cyclopropane, cyclobutane, cyclopentane, cyclohexane, norbornane and adamantane rings. Some or all of hydrogen atoms in the ring may be replaced by a group containing a heteroatom such as oxygen, sulfur, nitrogen or halogen, some constituent —CH₂-in the ring may be replaced by a group containing a heteroatom such as oxygen, sulfur or nitrogen, and as a result, the hydrocarbyl group may contain a hydroxy group, fluorine, chlorine, bromine, iodine, a cyano group, a carbonyl group, an ether bond, an ester bond, a sulfonic ester bond, a carbonate bond, a lactone ring, a sultone ring, carboxylic anhydride (—C(═O)—O—C(═O)—), a haloalkyl group, or the like.

In formula (d1), R³² is halogen exclusive of iodine, a C₁-C₂₀ hydrocarbyl group which may contain a heteroatom. Examples of the halogen exclusive of iodine include fluorine, chlorine, bromine, and iodine. The hydrocarbyl group may be saturated or unsaturated, and may be straight, branched, or cyclic. Examples of the hydrocarbyl group are as exemplified above as a hydrocarbyl group R³¹, but not limited thereto. A plurality of R³² may be identical or different when d4 is 2, 3 or 4.

A plurality of R³² may bond together to form a ring with the aromatic ring carbon atom to which they are attached when d4 is 2, 3 or 4. Of the rings, 5 to 8-membered rings are preferred.

In formula (d1), L^{C}, L^{D} and L^{E} are each independently a single bond, ether bond, ester bond, sulfonic ester bond, sulfonic amide bond, carbonate bond or carbamate bond. Inter alia, L^{C} is preferably a single bond, ether bond, ester bond or sulfonic ester bond, more preferably ester bond or sulfonic ester bond. L^{D} is preferably a single bond, an ether bond or ester bond, more preferably a single bond. L^{E} is preferably a single bond, ether bond, ester bond or sulfonic ester bond, more preferably ether bond or ester bond.

In formula (d1), X^{L3} is a single bond or C₁-C₄₀ hydrocarbylene group which may contain a heteroatom. The hydrocarbylene group may be straight, branched or cyclic, and examples thereof include alkanediyl, cyclic saturated hydrocarbylene, and arylene groups. Examples of the heteroatom include oxygen, nitrogen and sulfur atoms. Examples of the C₁-C₄₀ hydrocarbylene group X^{L3} which may contain a heteroatom include X^{L}-0 to X^{L}-58 exemplified for formula (Z) as examples of the C₁-C₄₀ hydrocarbylene groups X^{L1} and X^{L2} which may contain a heteroatom. Of these, X^{L}-0 to X^{L}-22, X^{L}-29 to X^{L}-34, and X^{L}-47 to X^{L}-58 are preferred.

X^{L3} is preferably a single bond from the aspect of rigidity of the polymer obtained.

In formula (d1), Q¹ and Q² are each independently hydrogen, fluorine or a C₁-C₆ fluorinated saturated hydrocarbyl group. The C₁-C₆ fluorinated saturated hydrocarbyl group is preferably a trifluoromethyl group.

In formula (d1), Q³ and Q⁴ are each independently fluorine or a C₁-C₆ fluorinated saturated hydrocarbyl group. The C₁-C₆ fluorinated saturated hydrocarbyl group is preferably a trifluoromethyl group. More preferably, Q³ and Q⁴ are fluorine.

Preferred examples of the partial structure —[C(Q¹)(Q²)]_{d6}-C(Q³)(Q⁴)-SO₃⁻ in formula (d1) are shown below, but the partial structure is not limited thereto. In the following formulae, * designates a point of attachment to L^{C}.

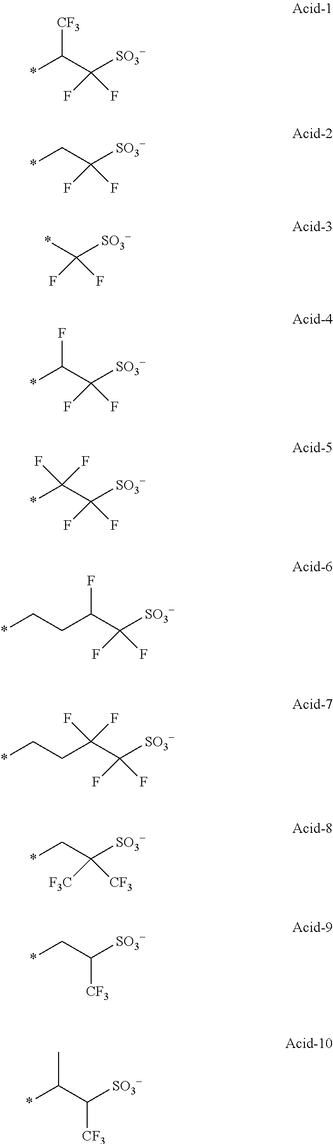

Of these, Acid-1 to Acid-7 are preferred, and Acid-1 to Acid-3, Acid-6 and Acid-7 are more preferred.

Of the anions of formula (d1), a structure having the formula (d1-1) is preferred.

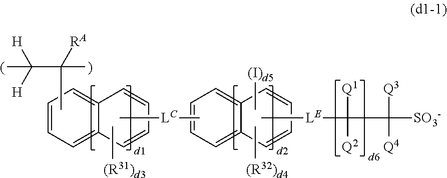

Herein, d1 to d6, R^{A}, R³¹, R³², L^{C}, L^{E}, Q¹ to Q⁴, and Z⁺ are as defined above.

Of the anions of formula (d1-1), a structure having formula (d1-2) is preferred.

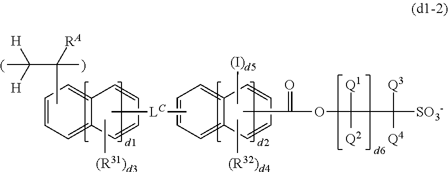

Herein, d1 to d6, R^{A}, R³¹, R³², L^{C}, Q¹, Q² and Z⁺ are as defined above.

Examples of the anion having formula (d1) are shown below, but not limited thereto. In the following formulae, R^{A} and Q¹ are as defined above, and Me is a methyl group. The bonding positions of the substituents on the aromatic ring may be interchanged.

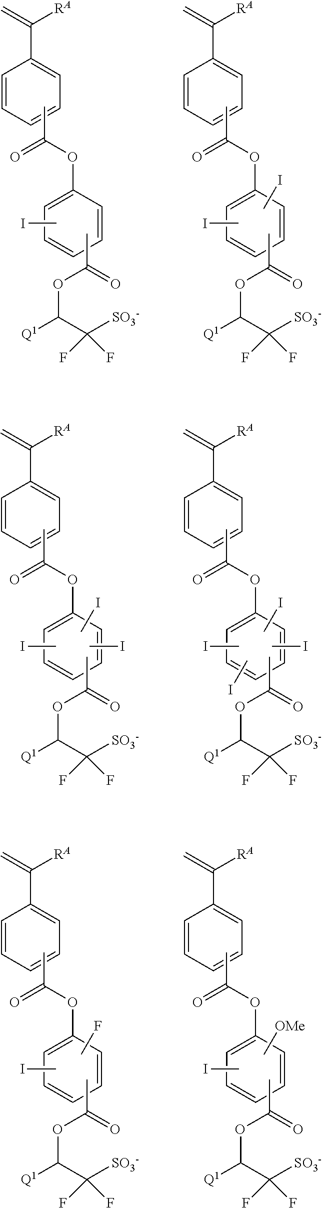

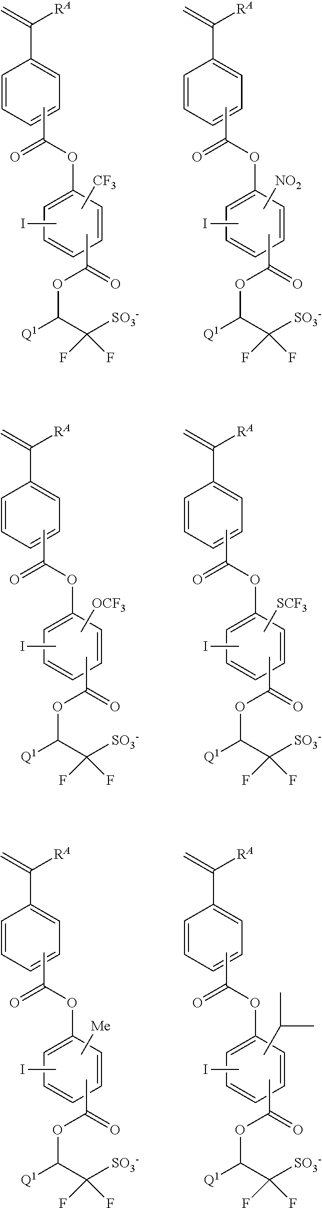

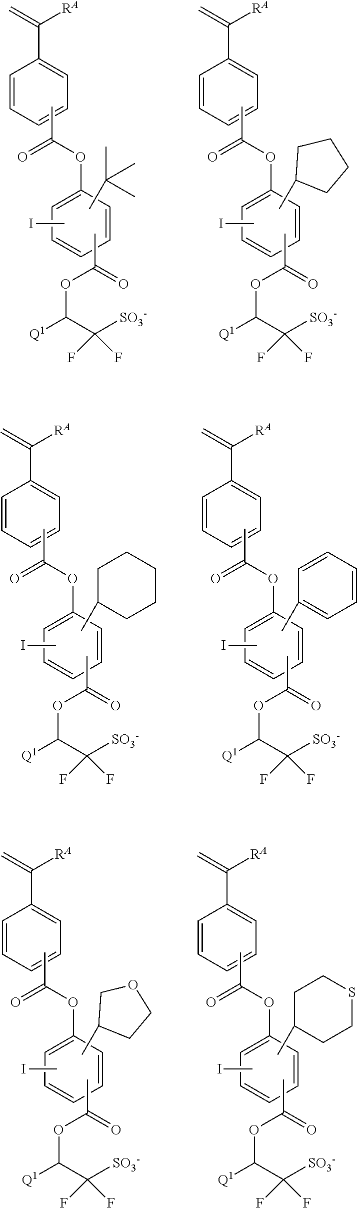

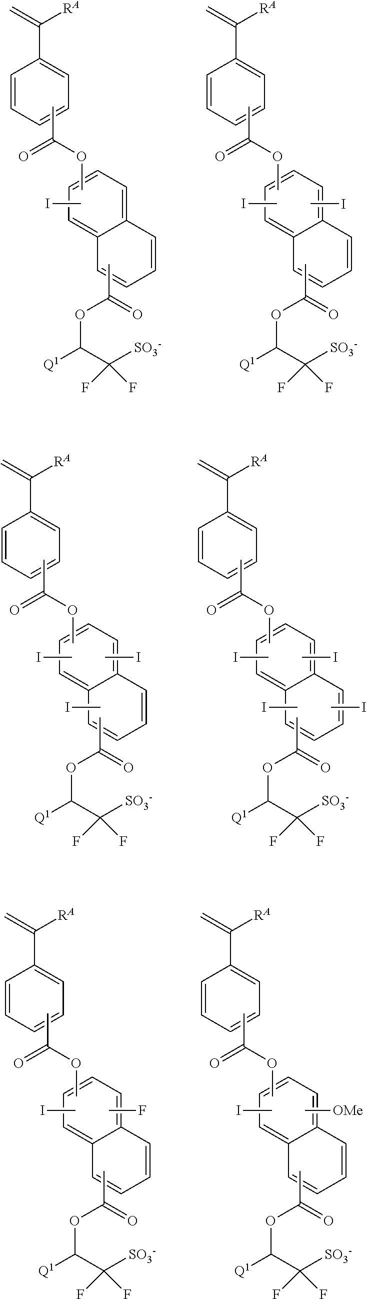

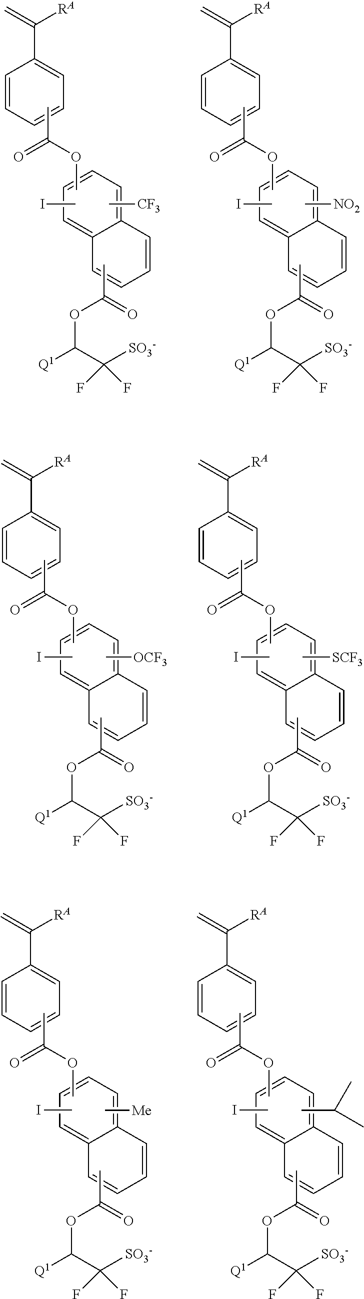

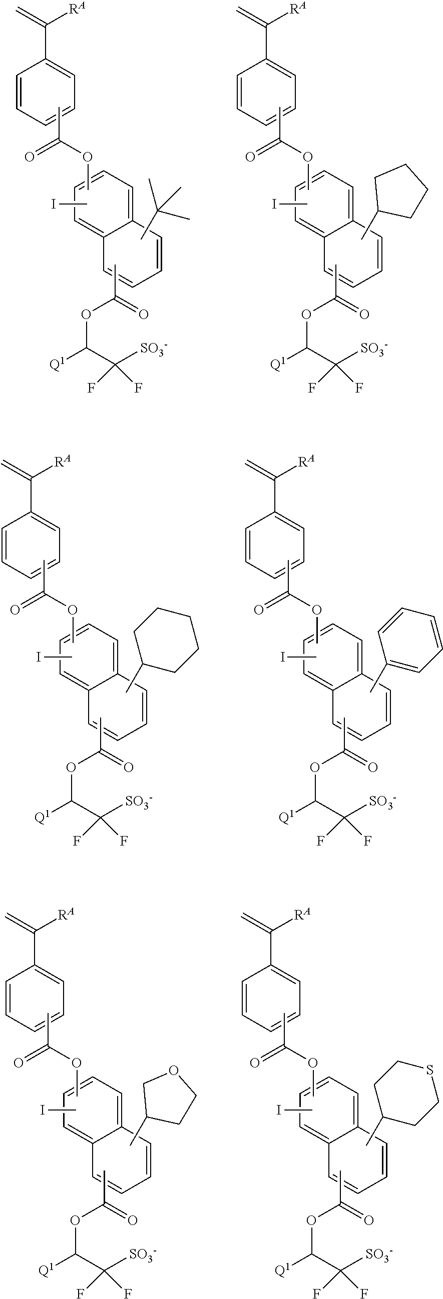

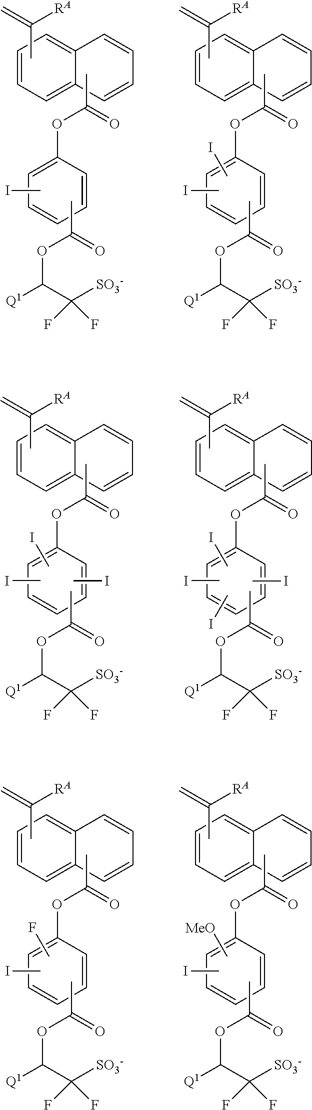

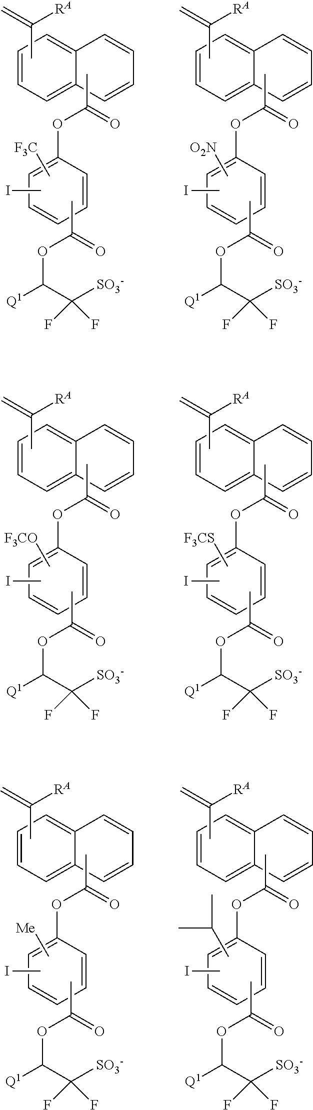

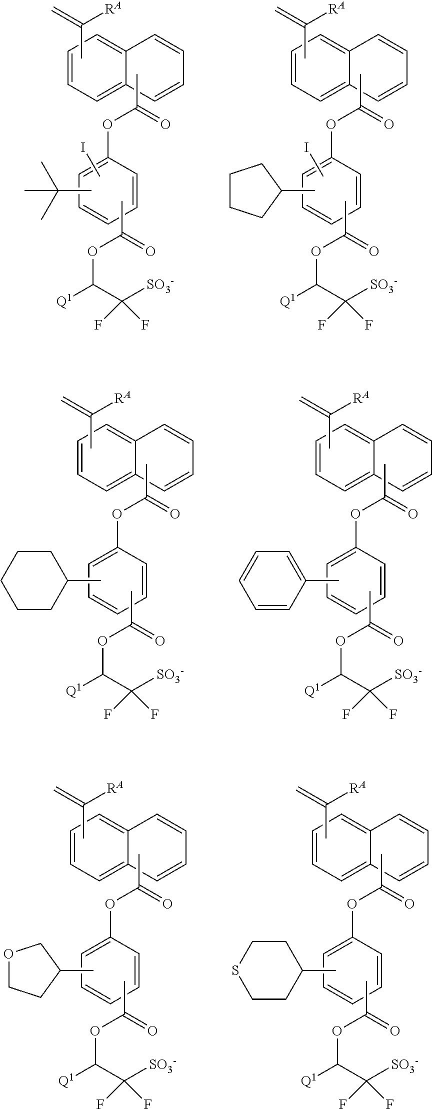

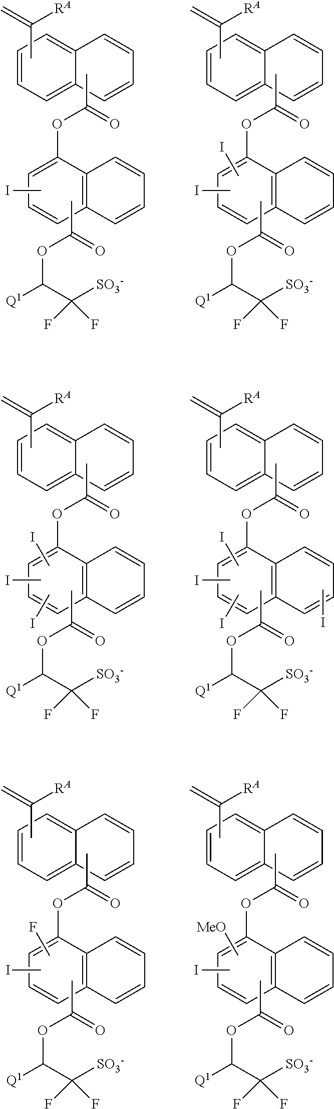

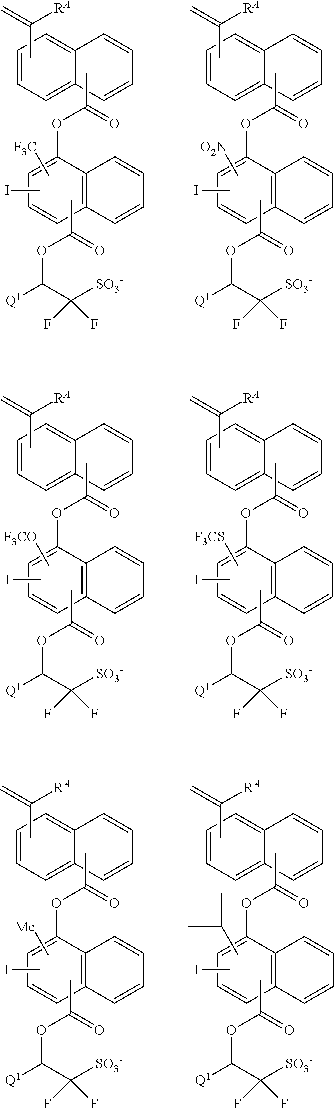

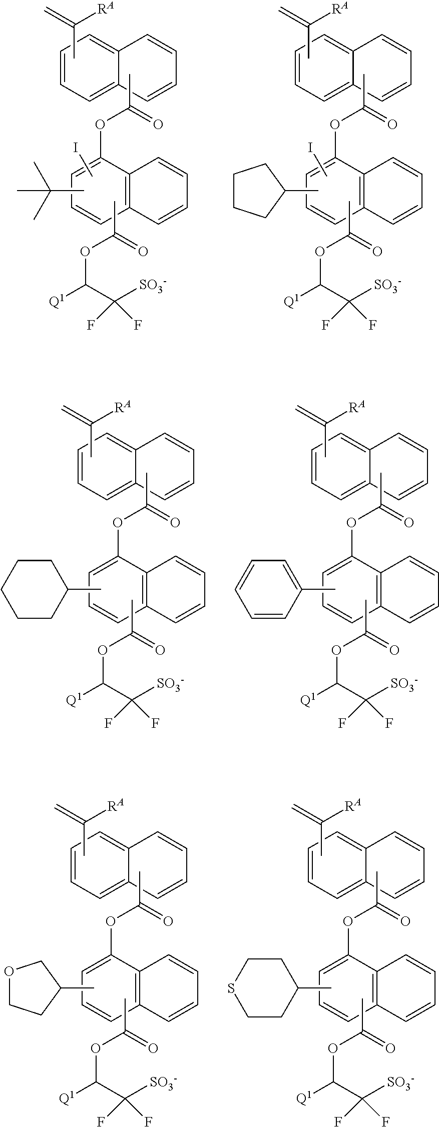

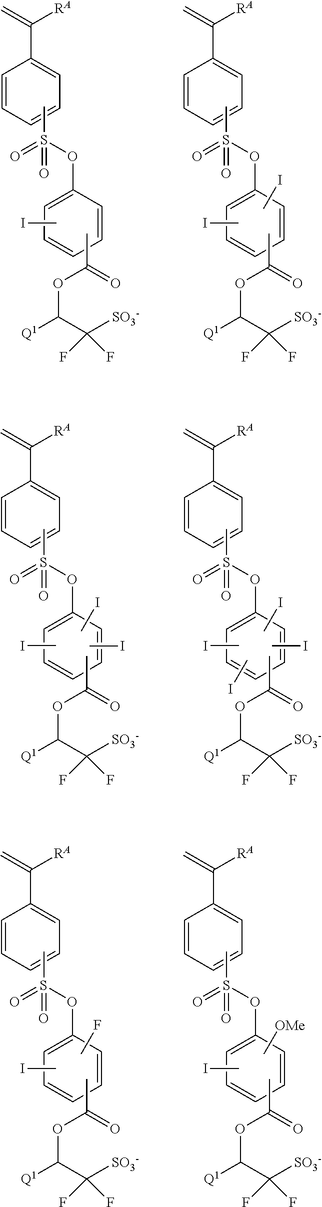

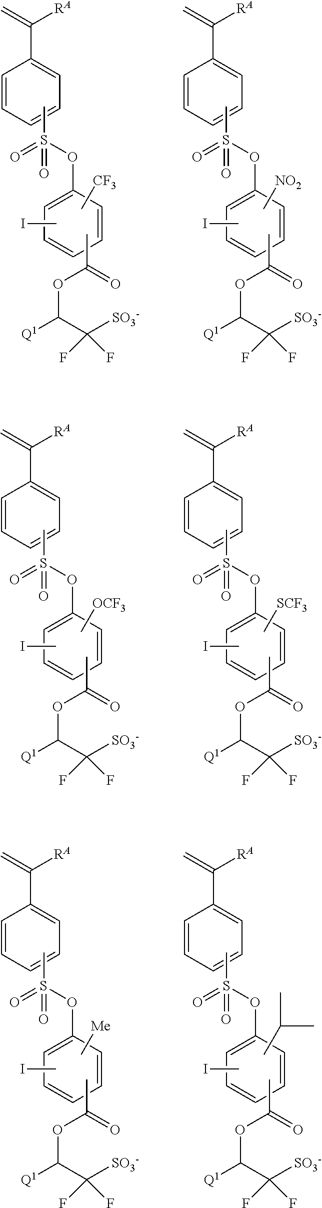

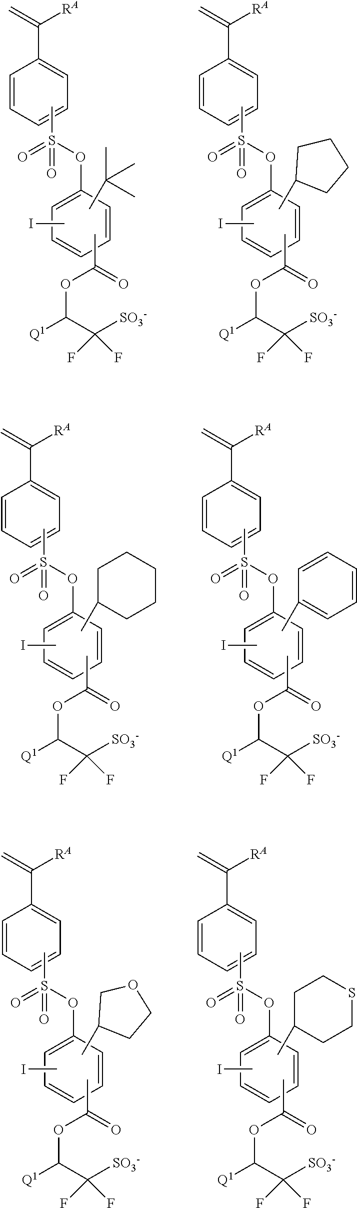

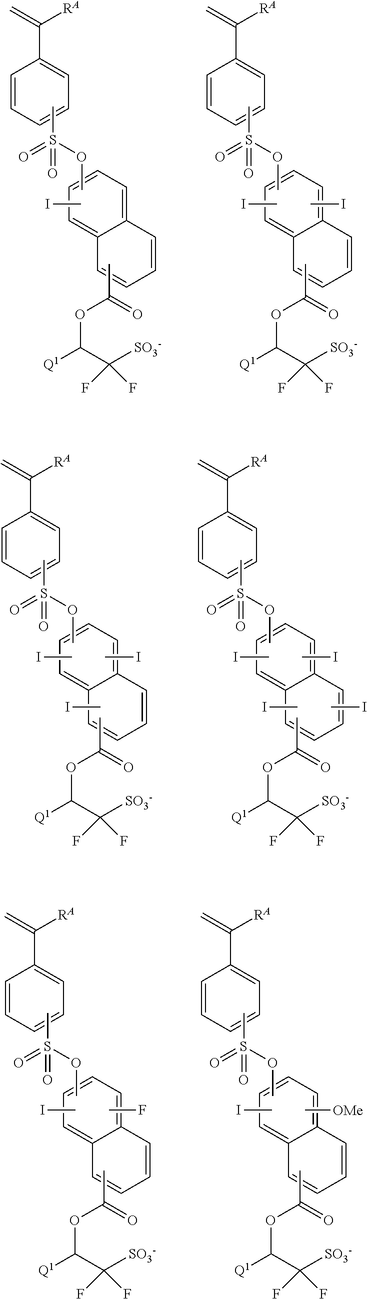

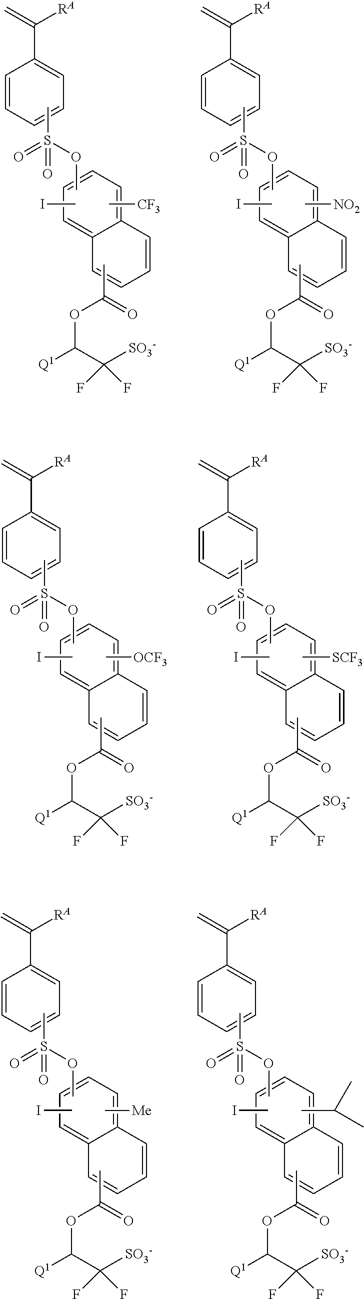

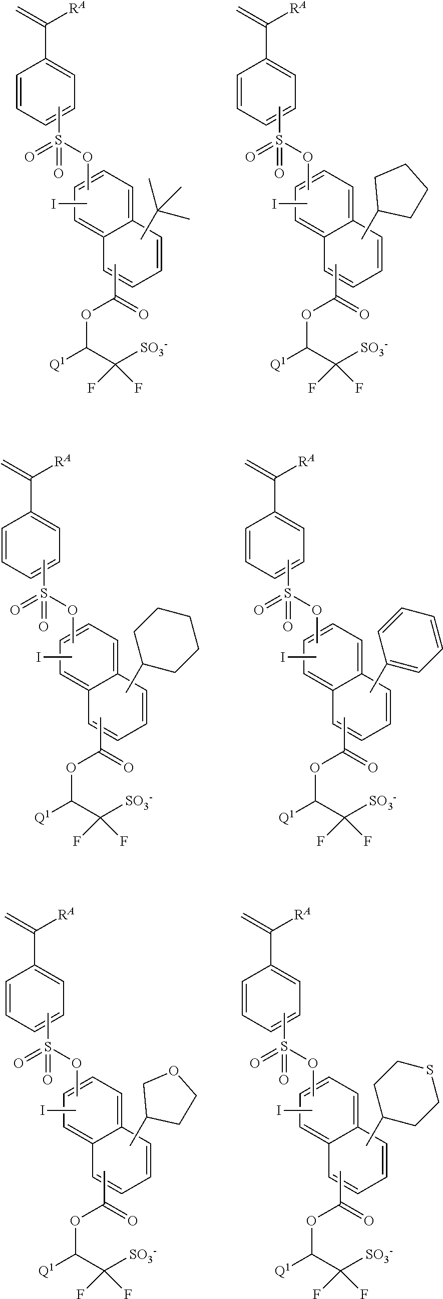

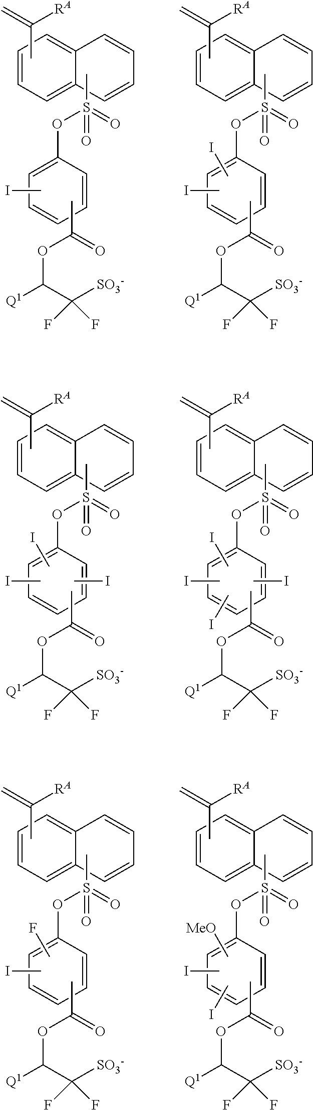

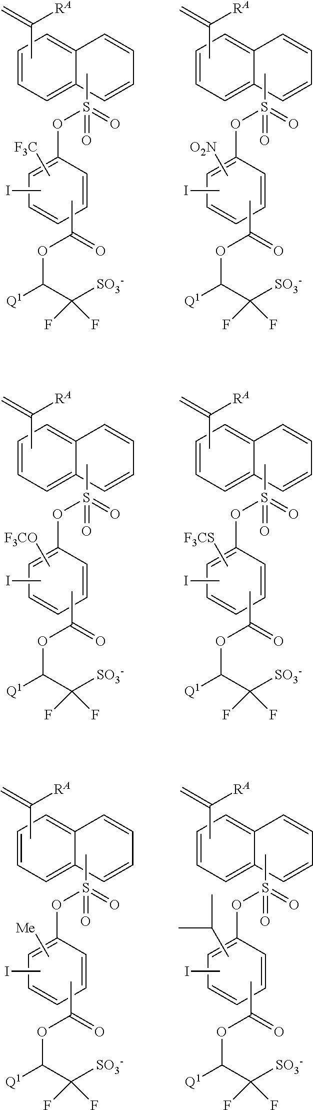

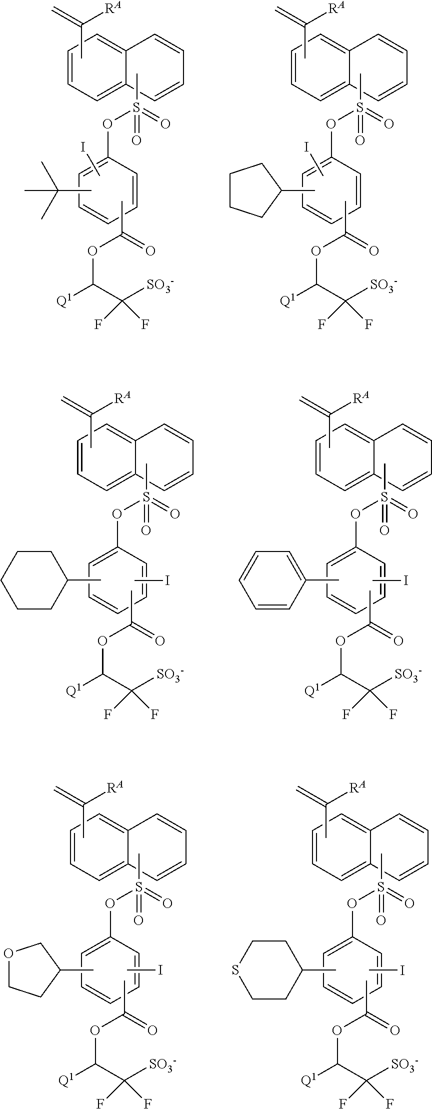

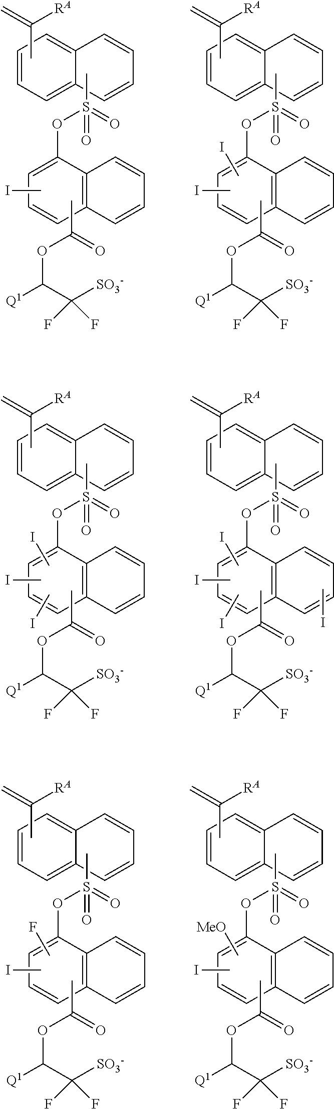

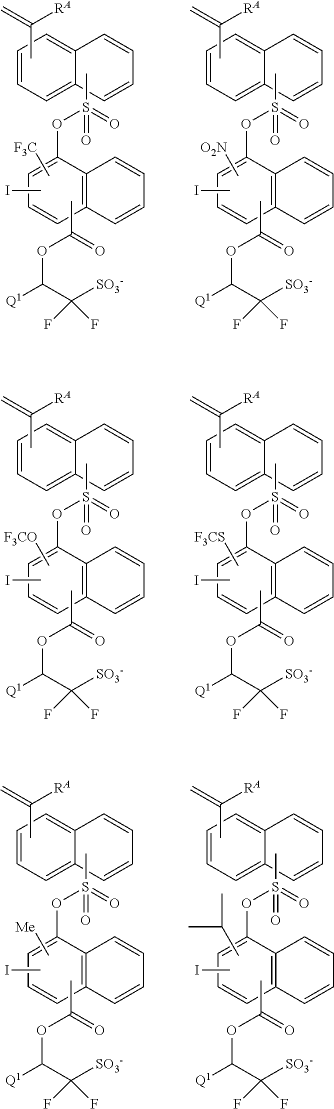

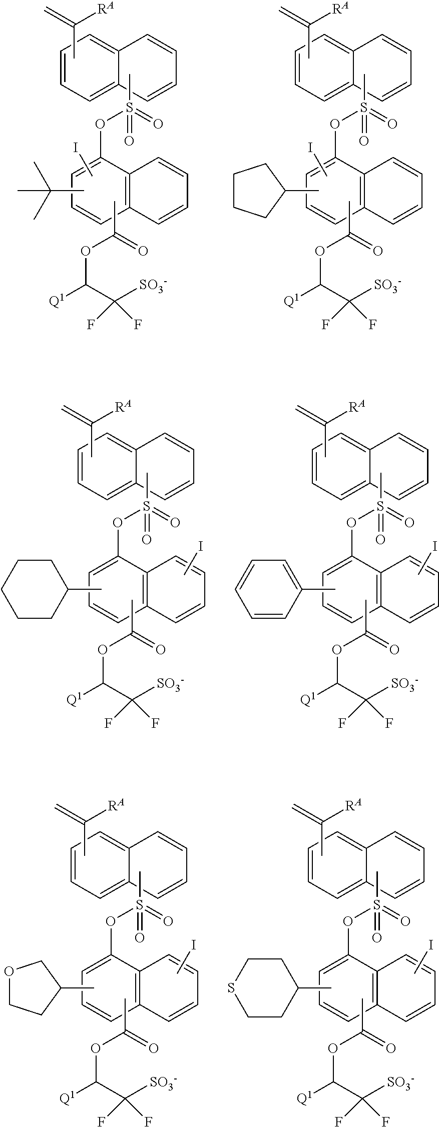

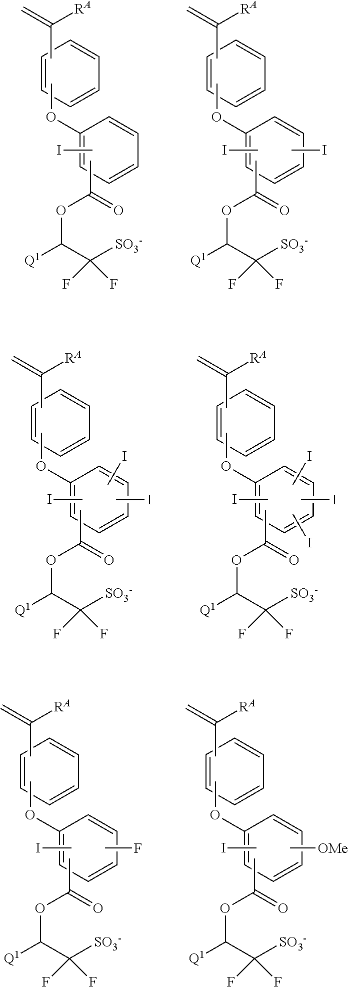

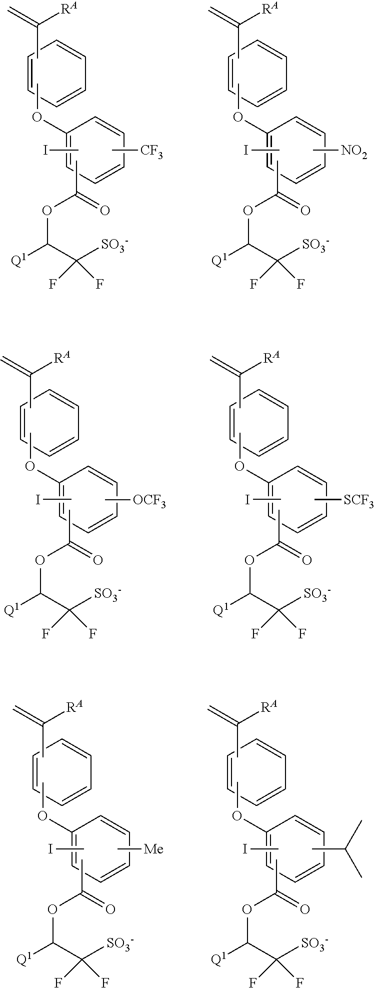

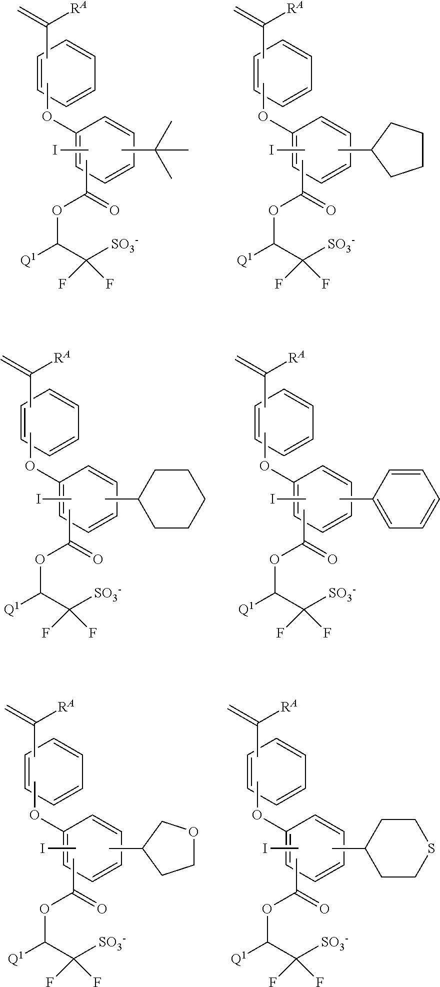

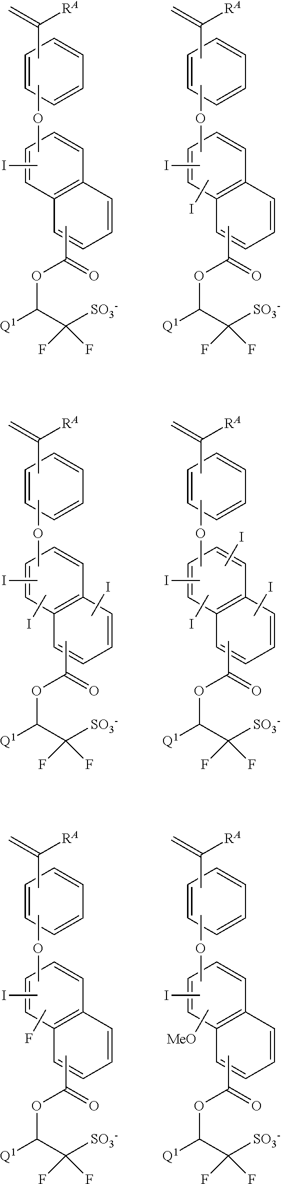

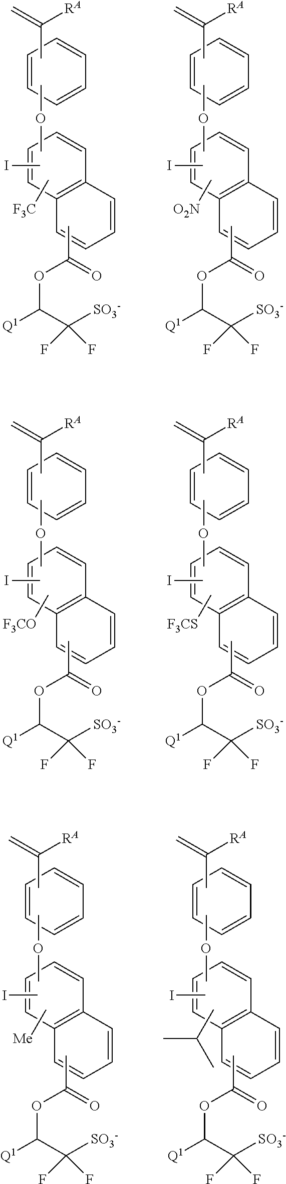

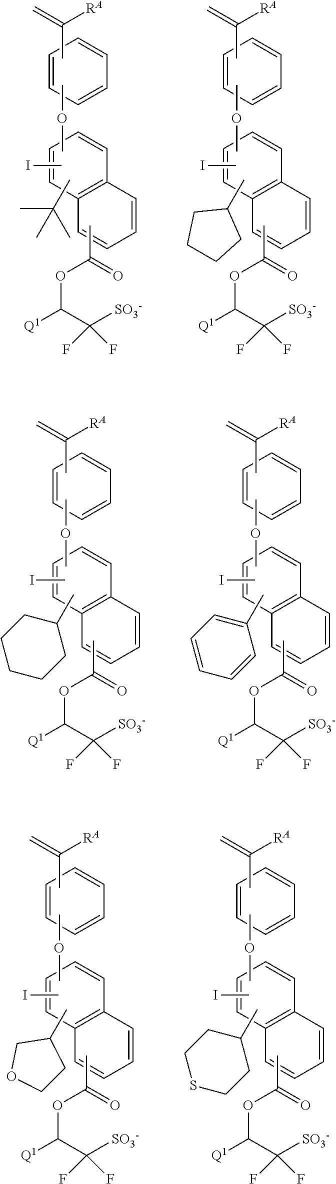

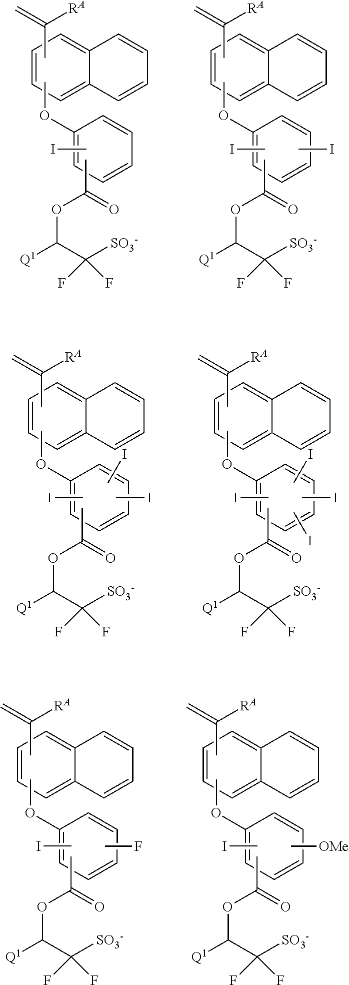

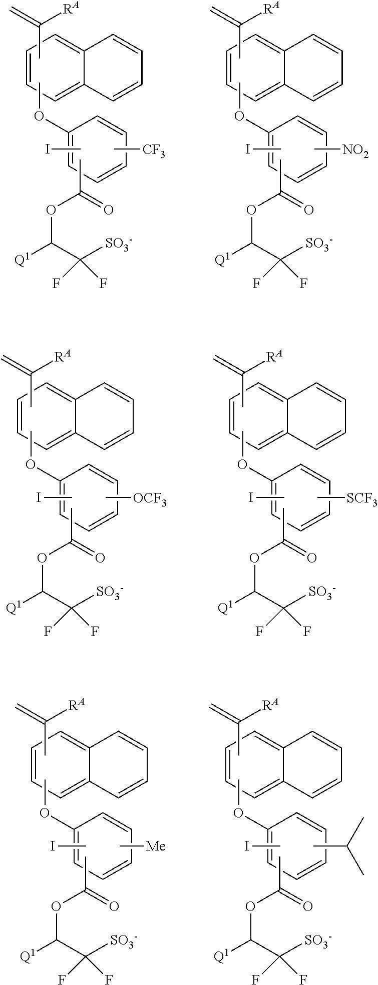

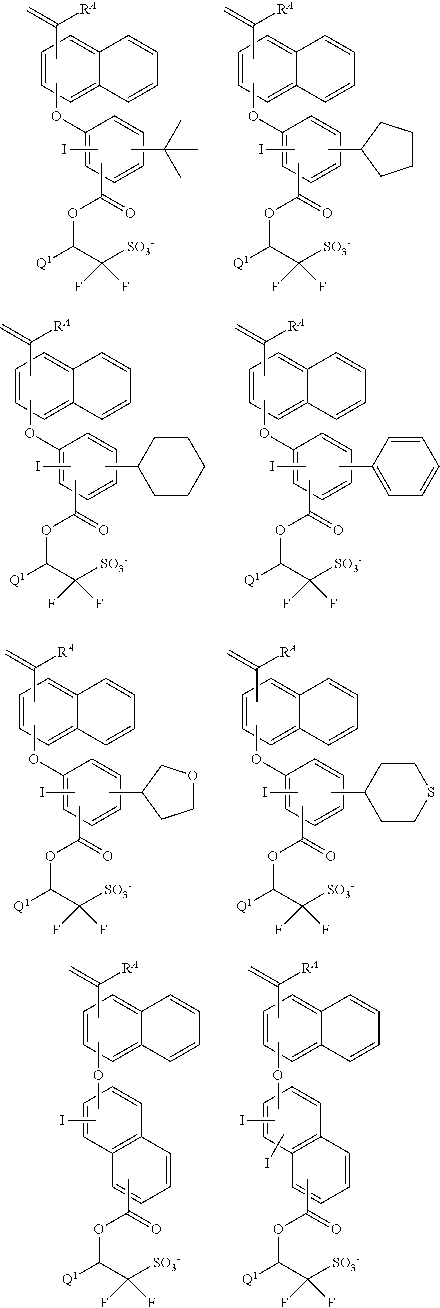

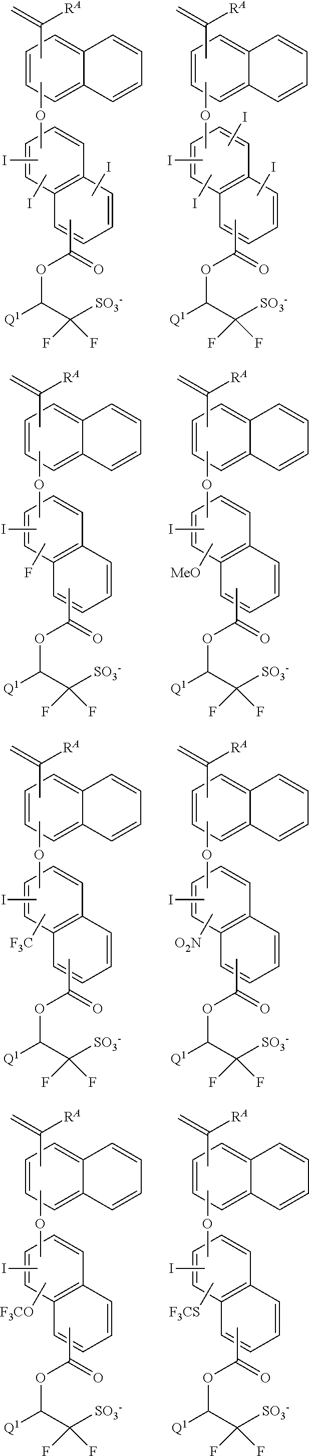

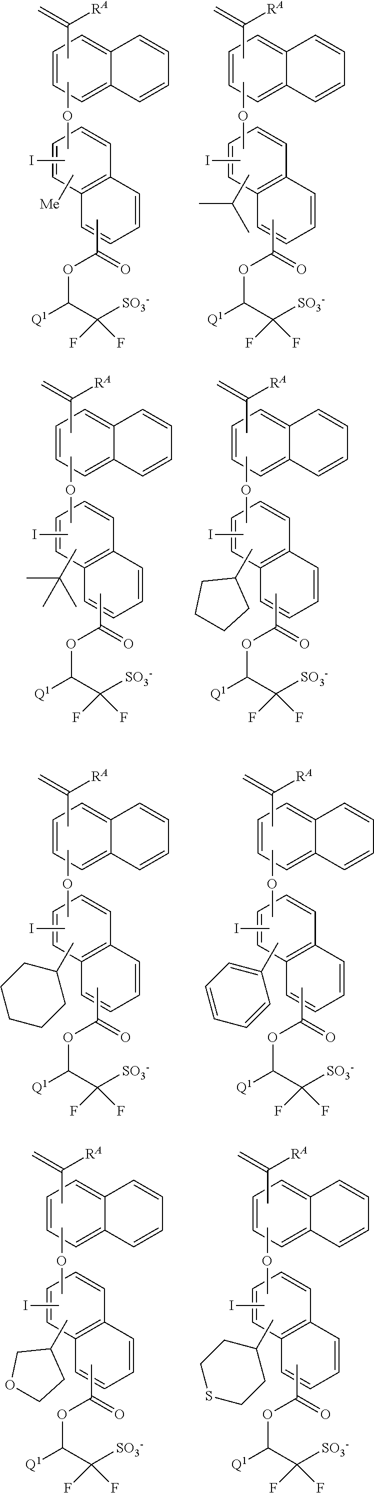

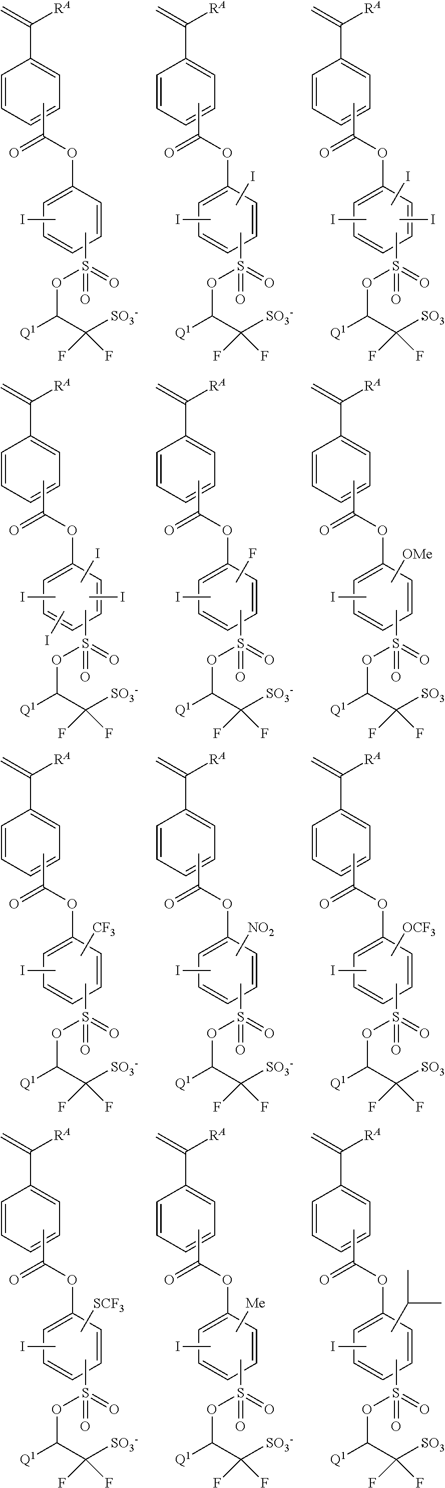

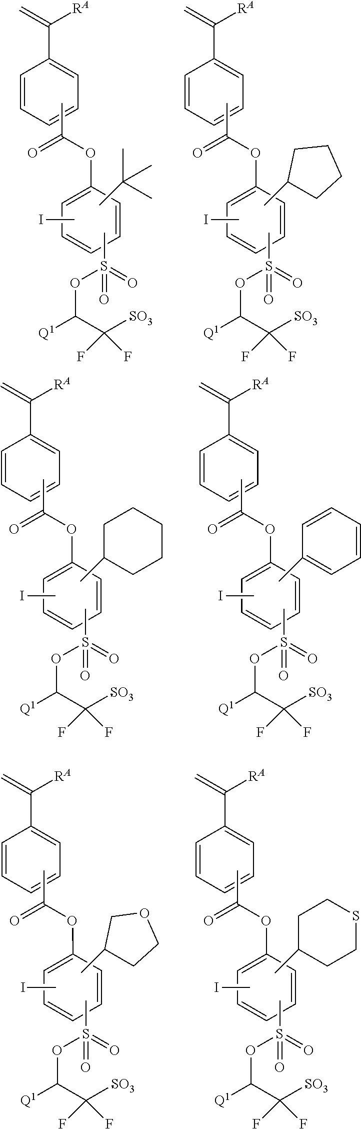

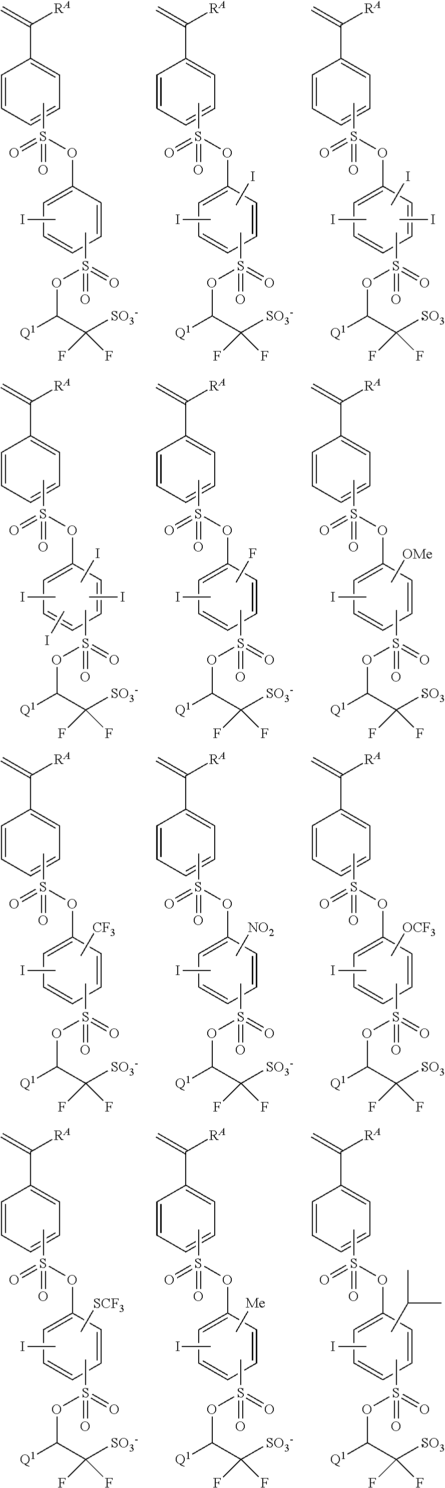

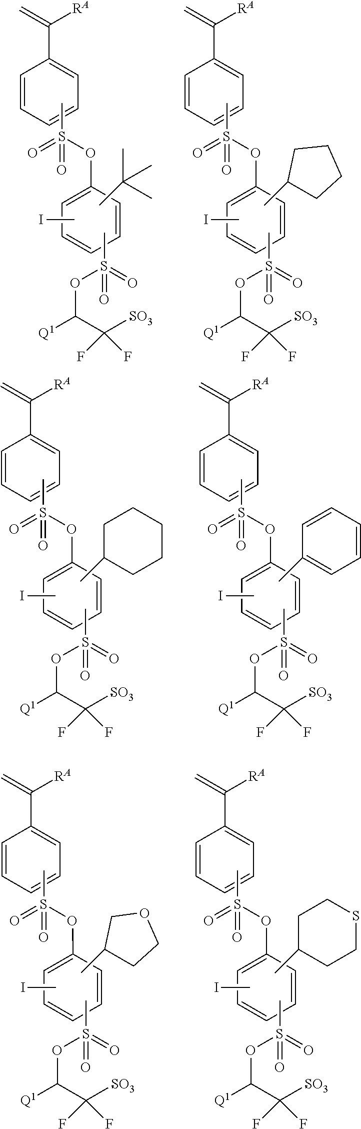

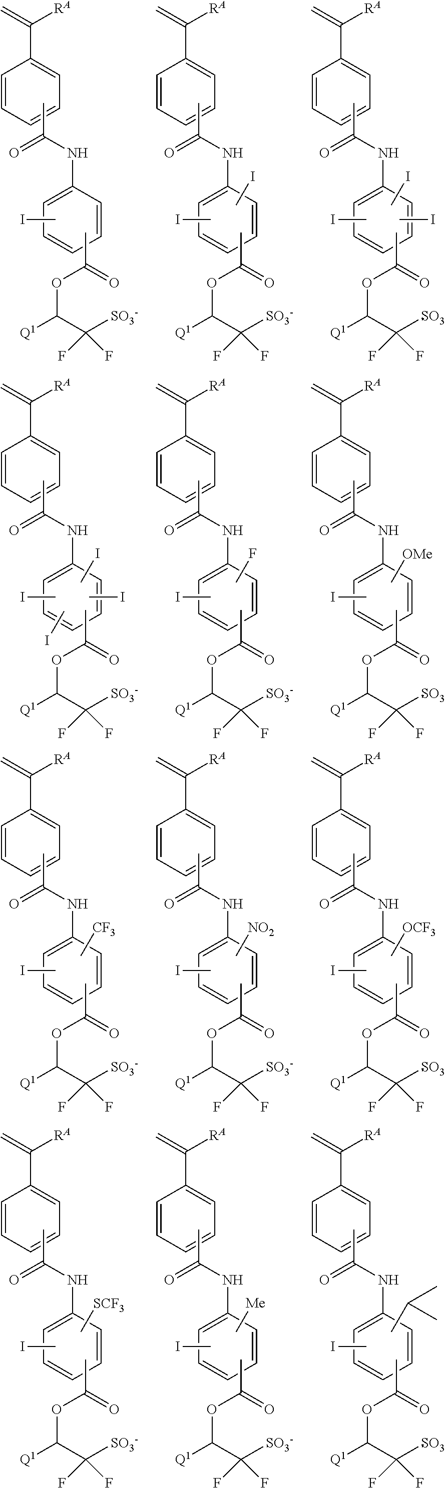

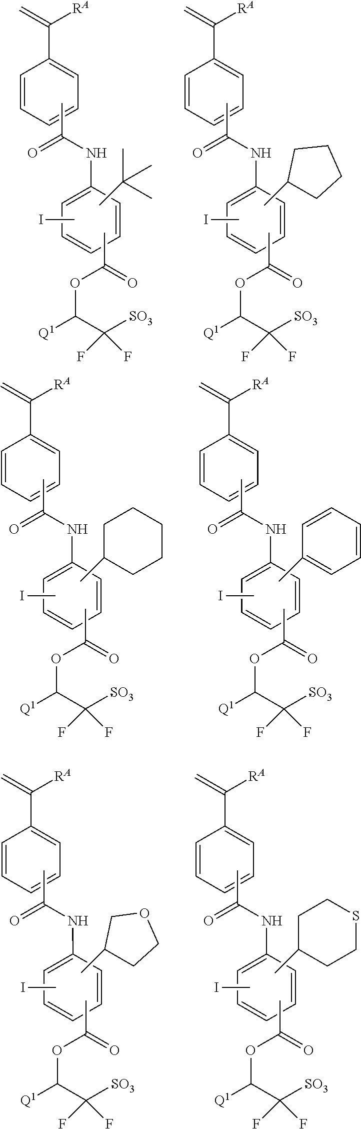

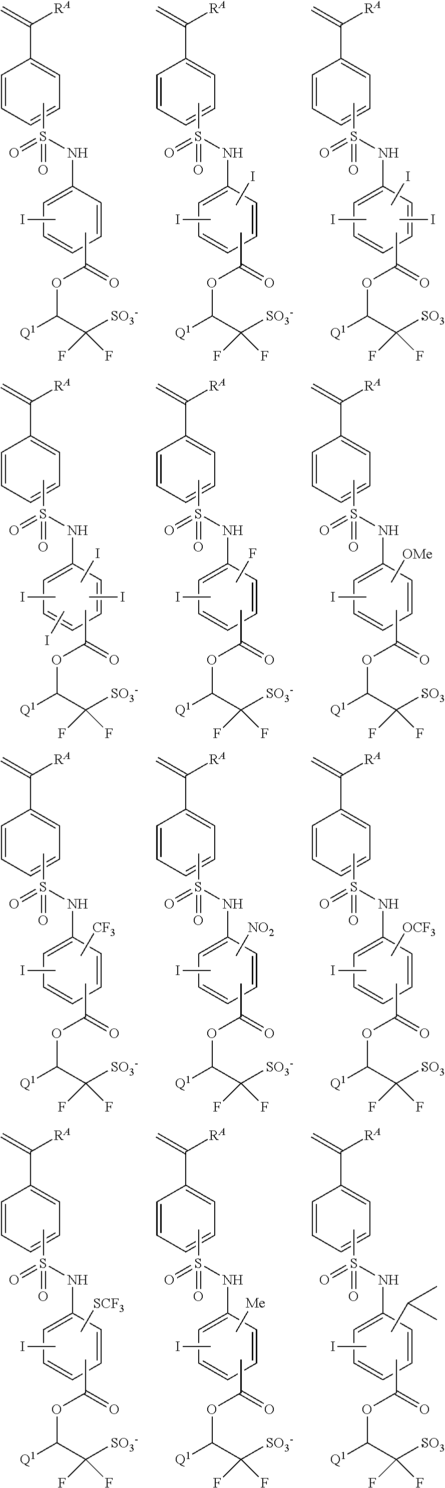

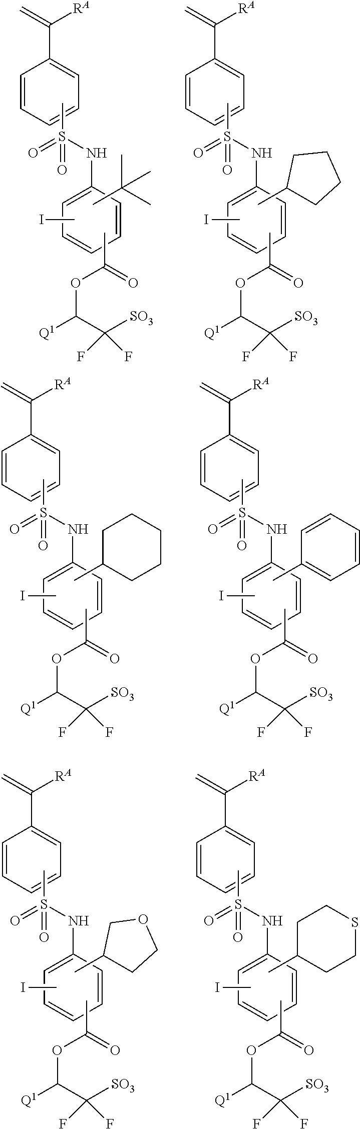

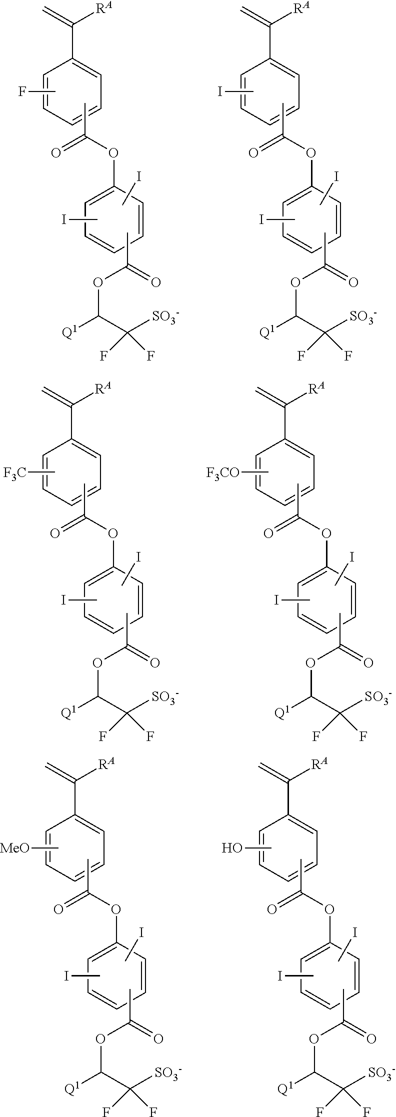

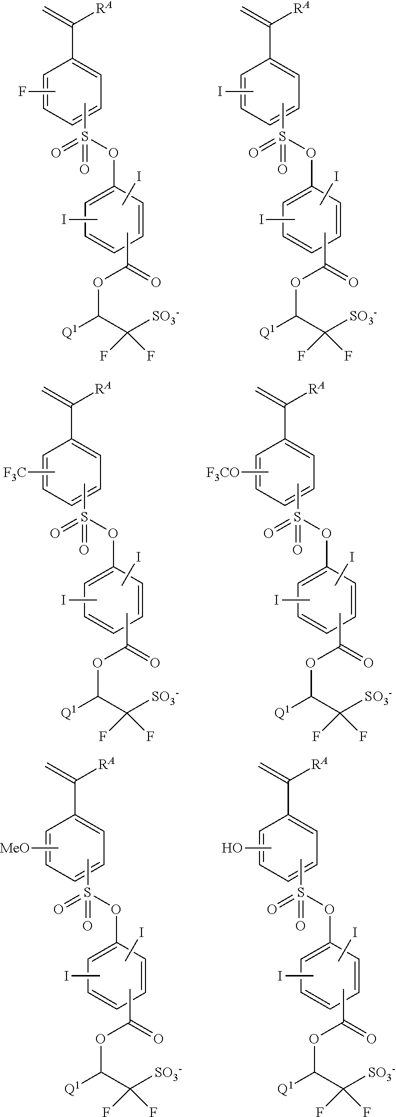

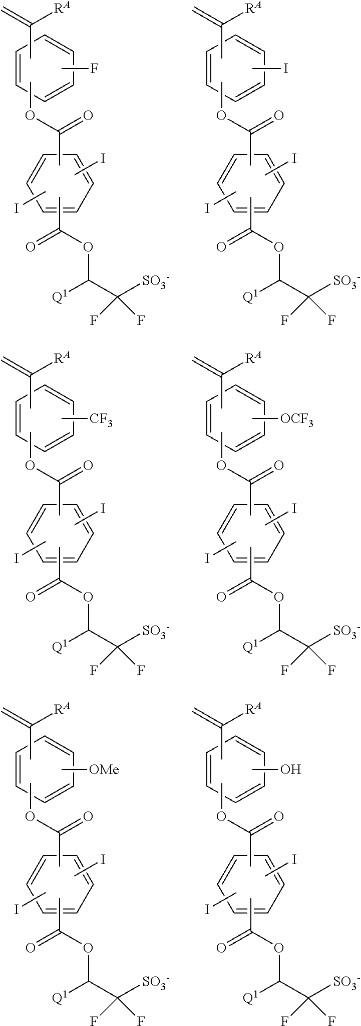

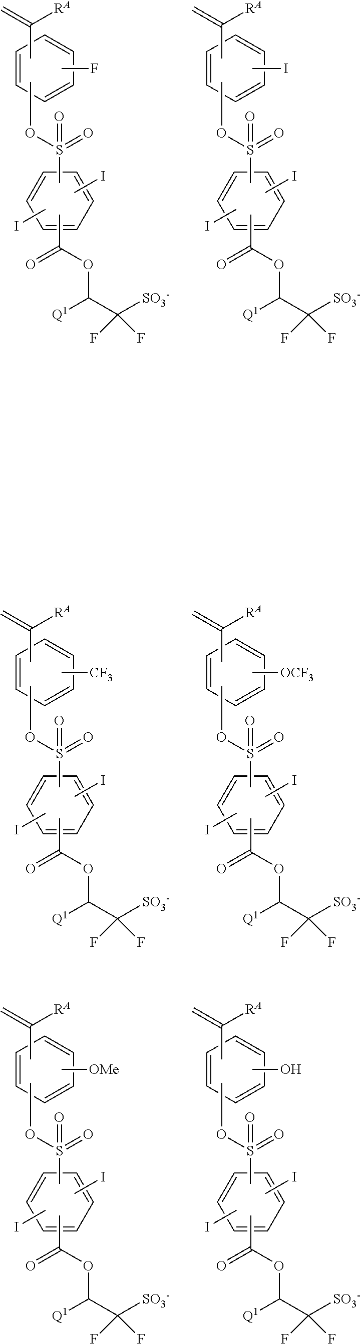

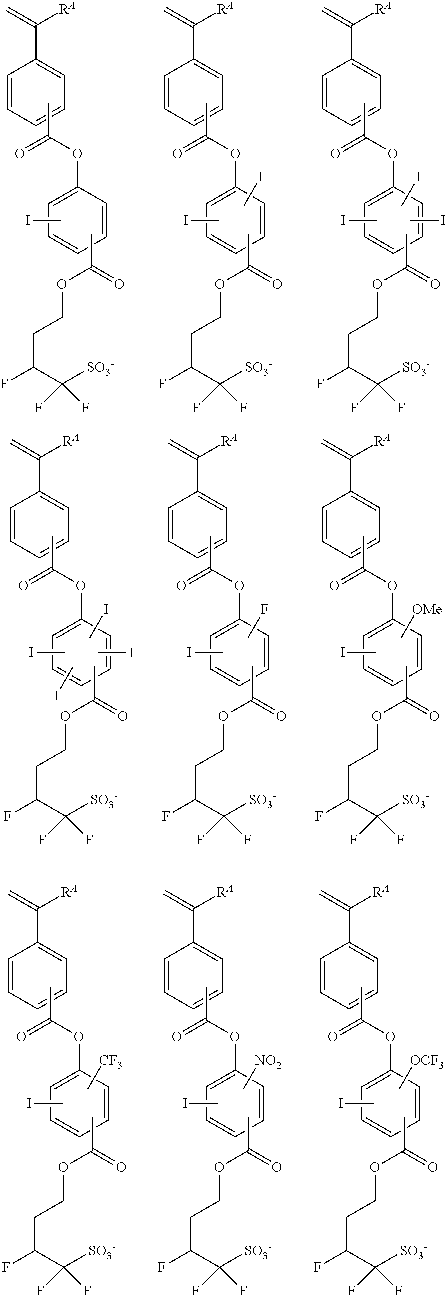

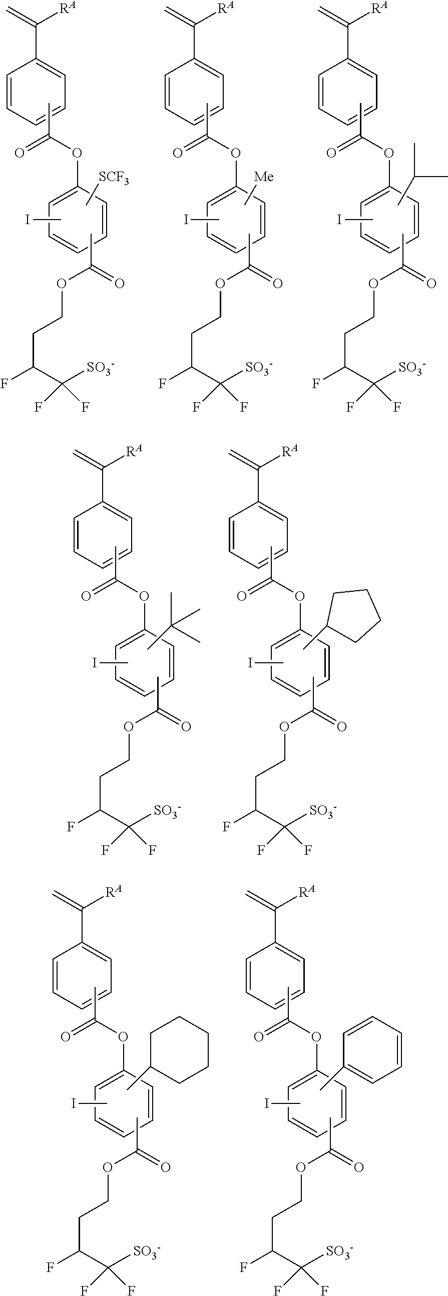

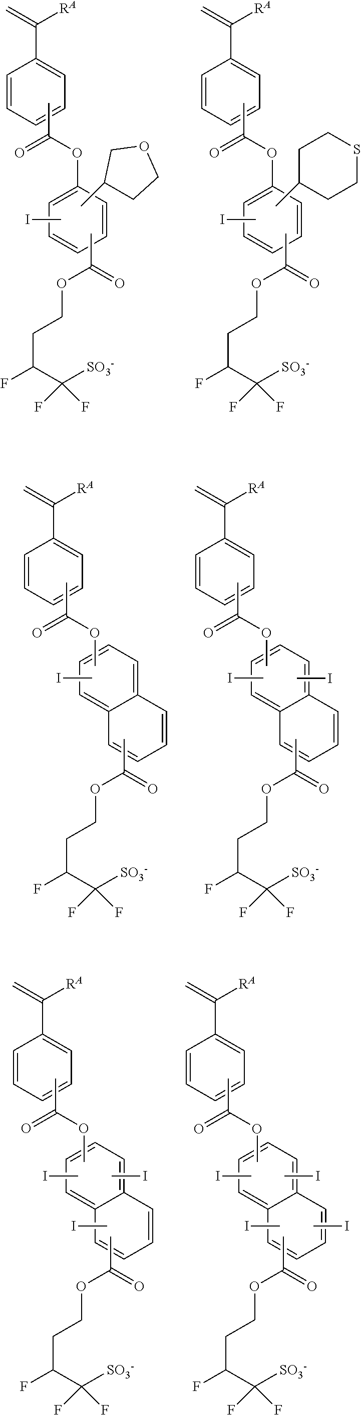

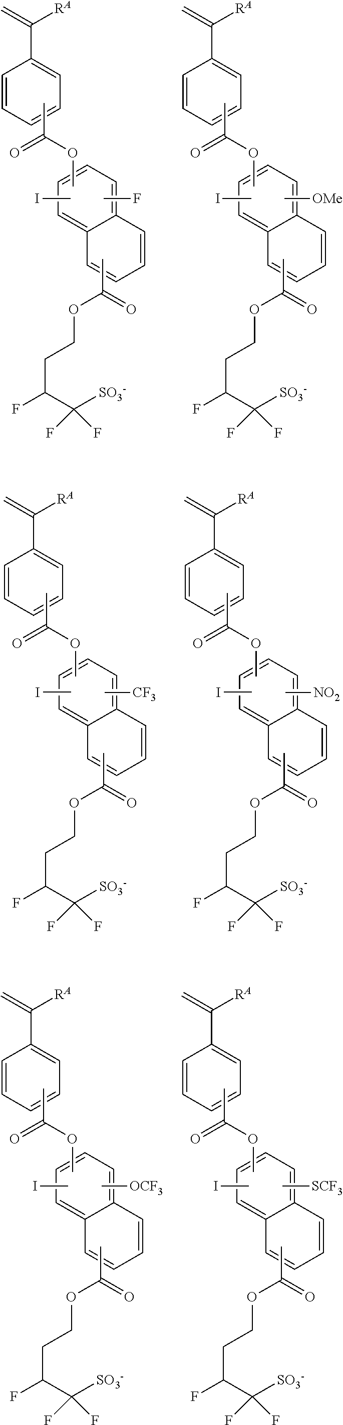

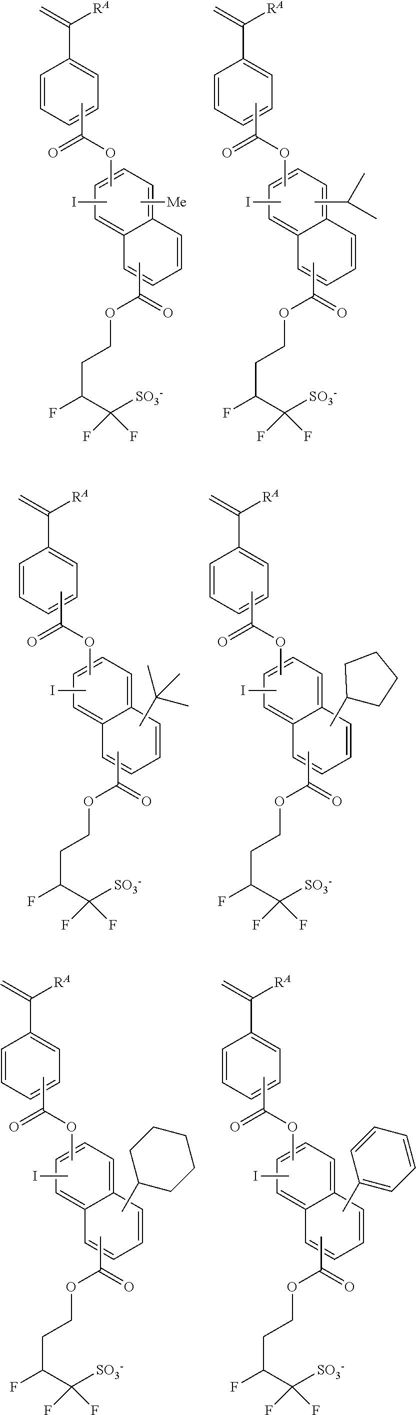

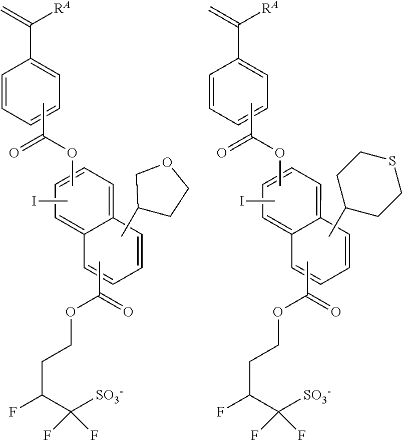

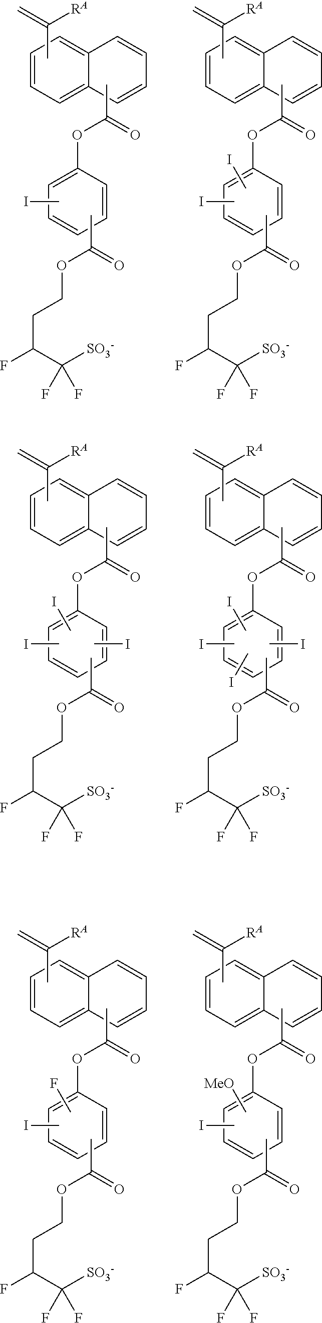

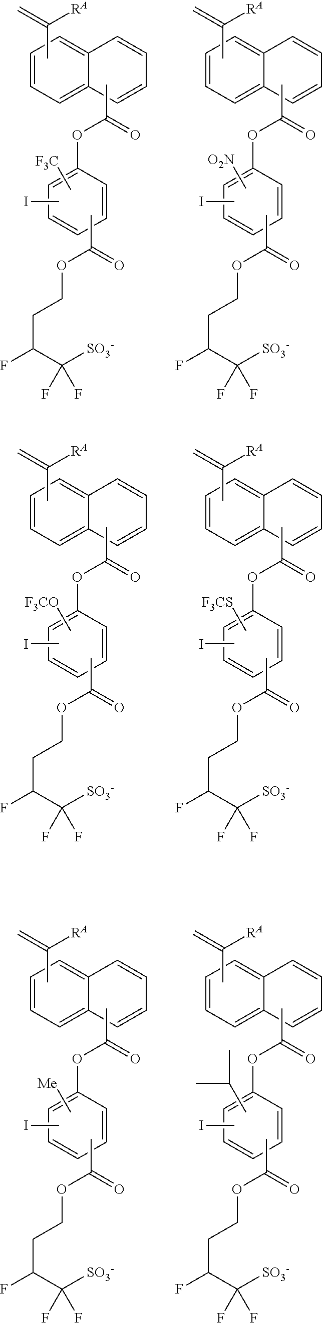

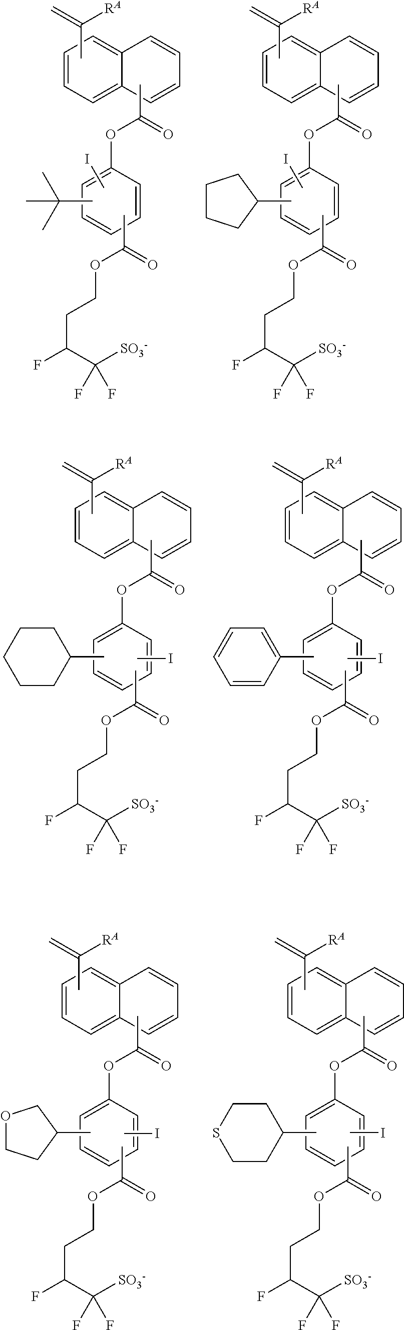

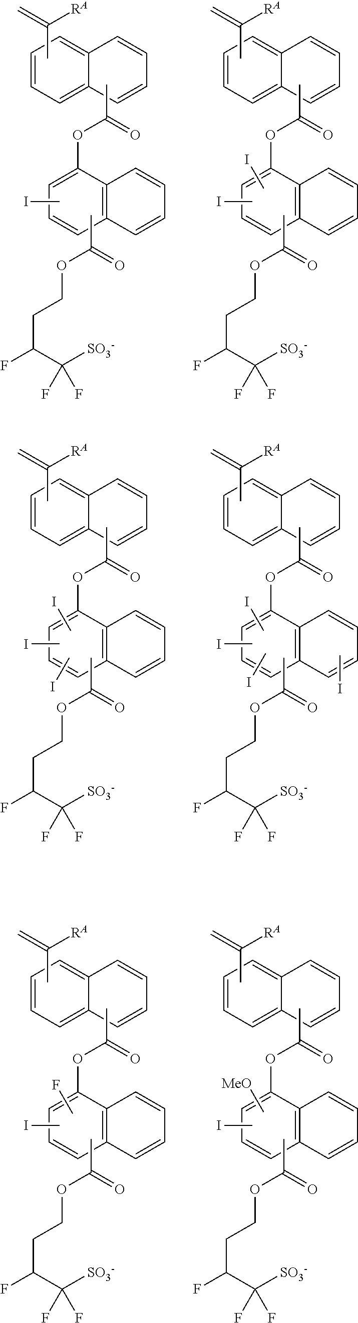

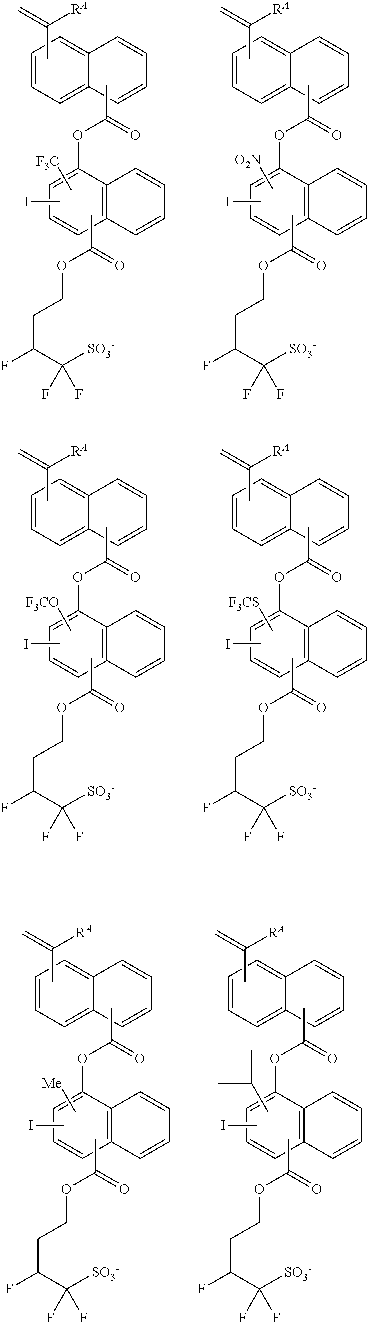

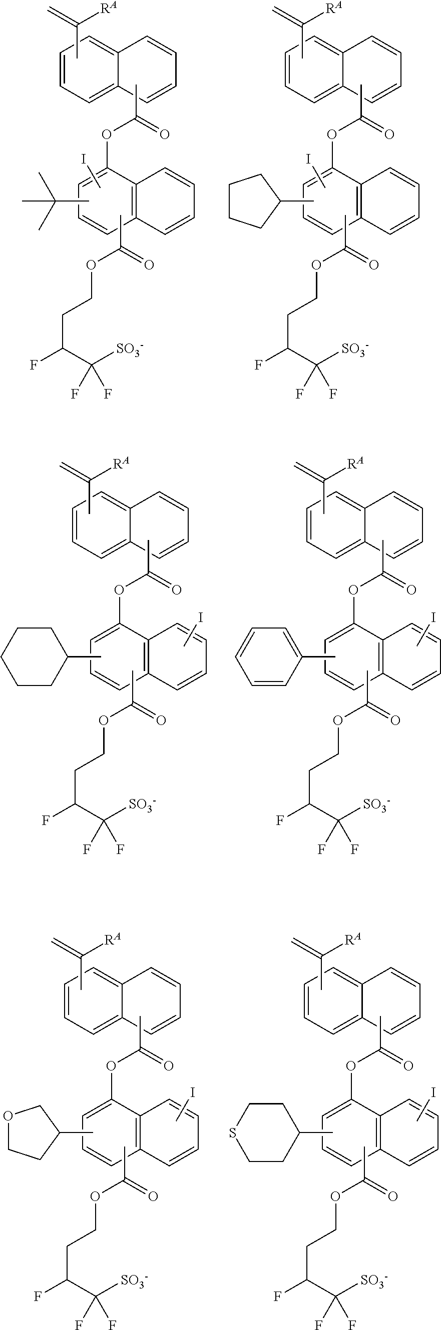

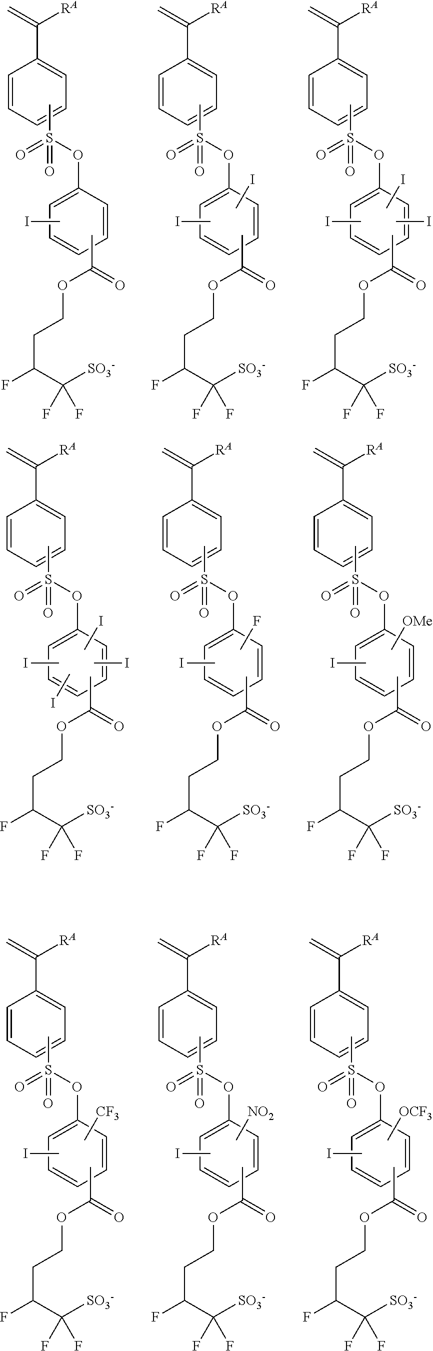

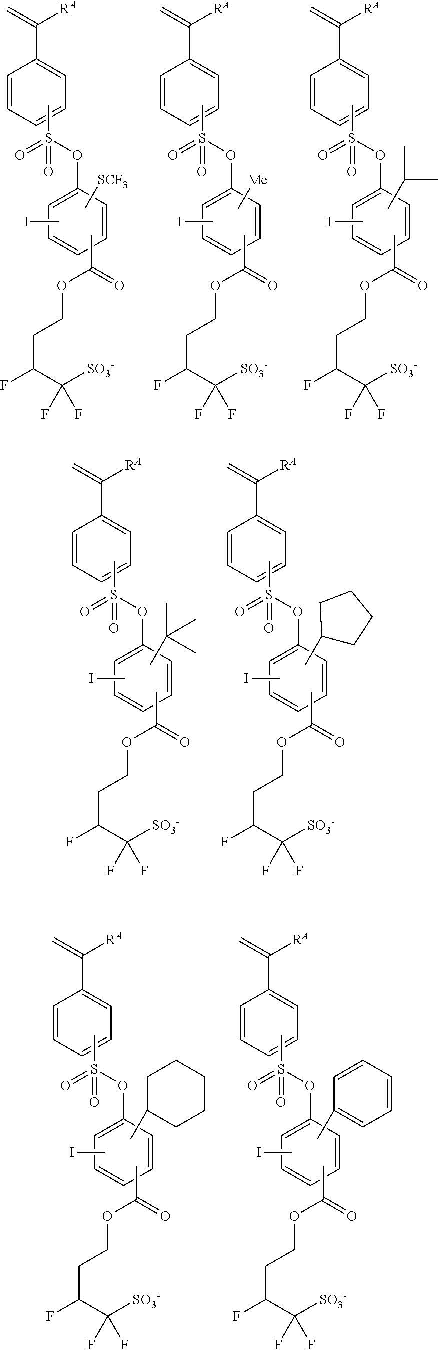

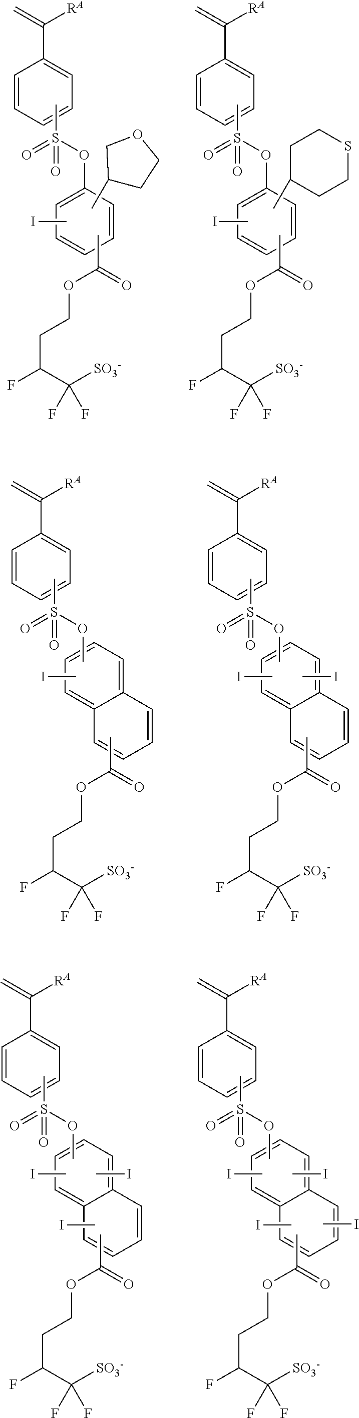

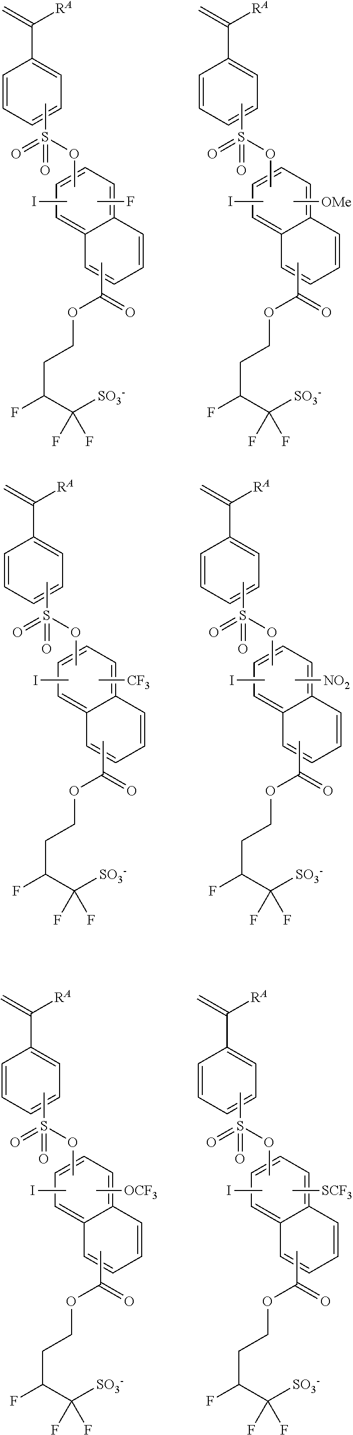

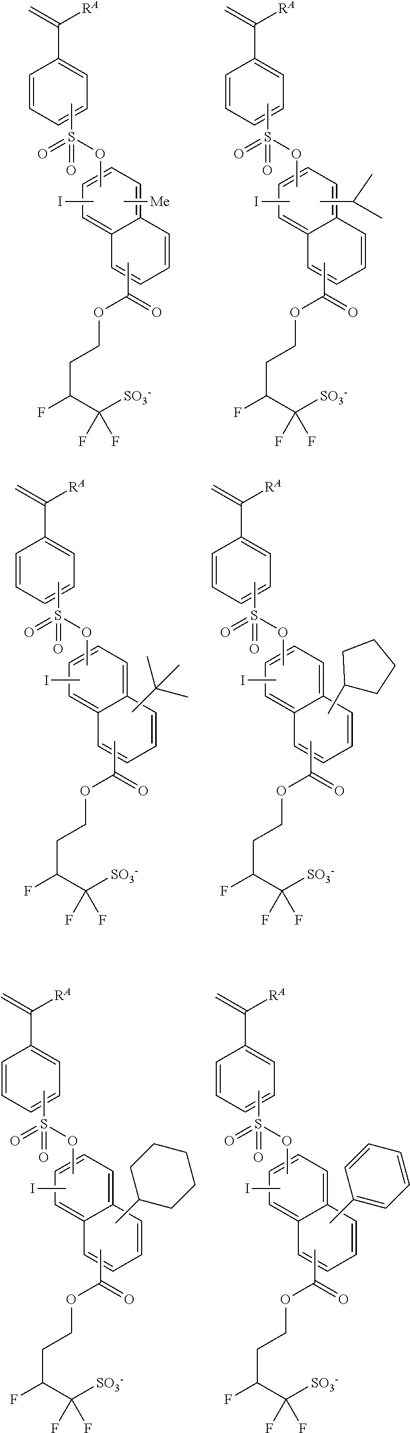

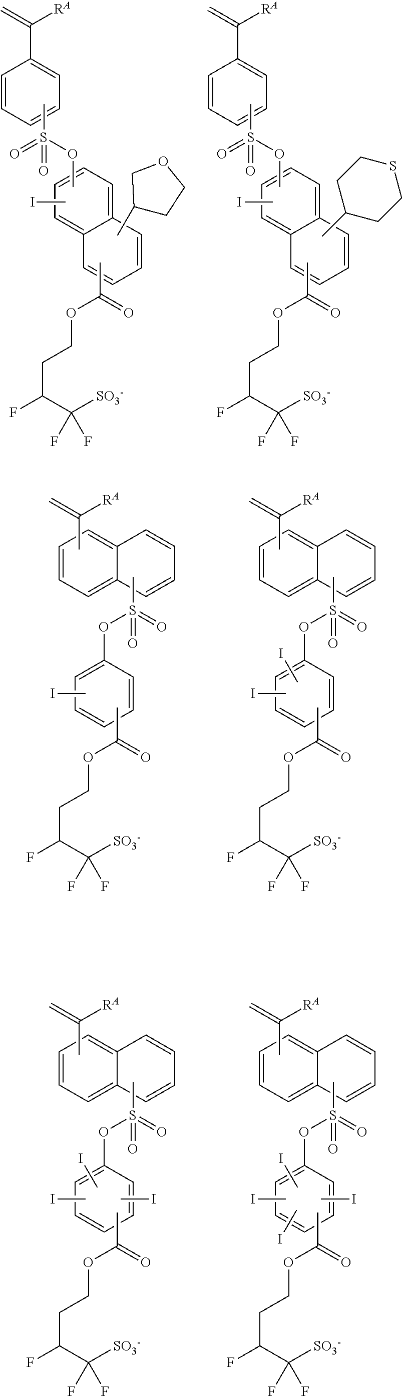

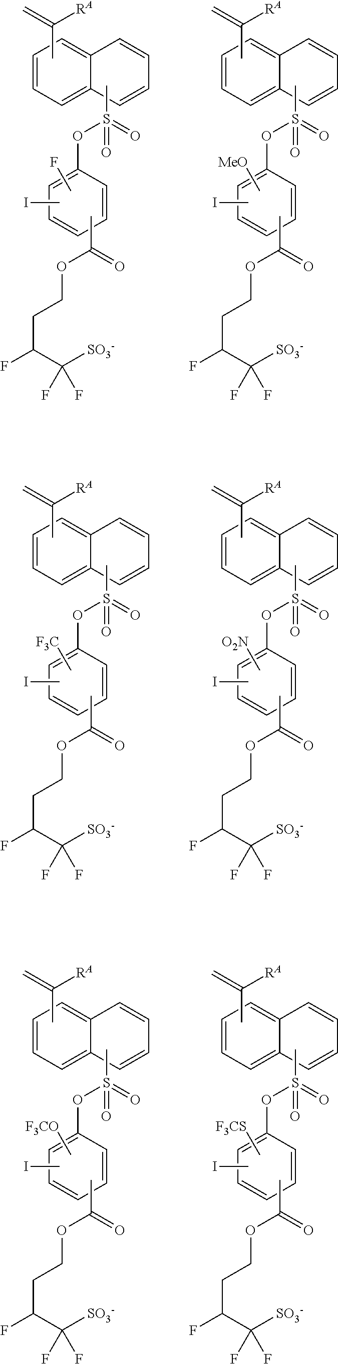

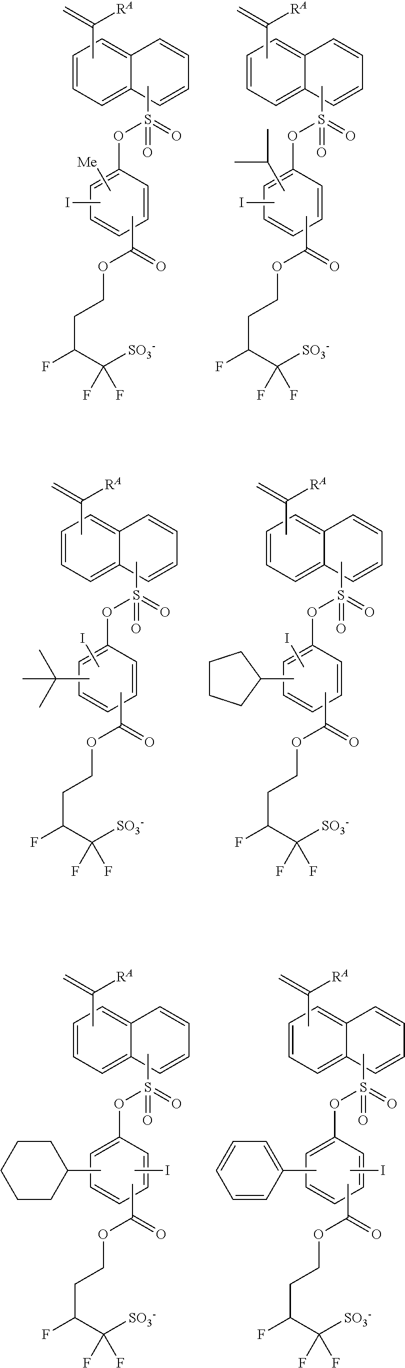

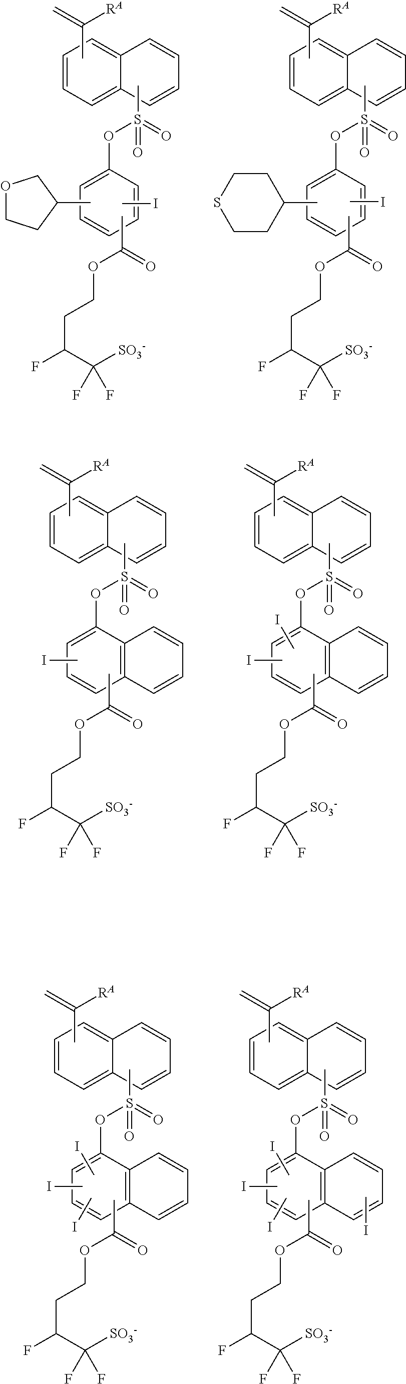

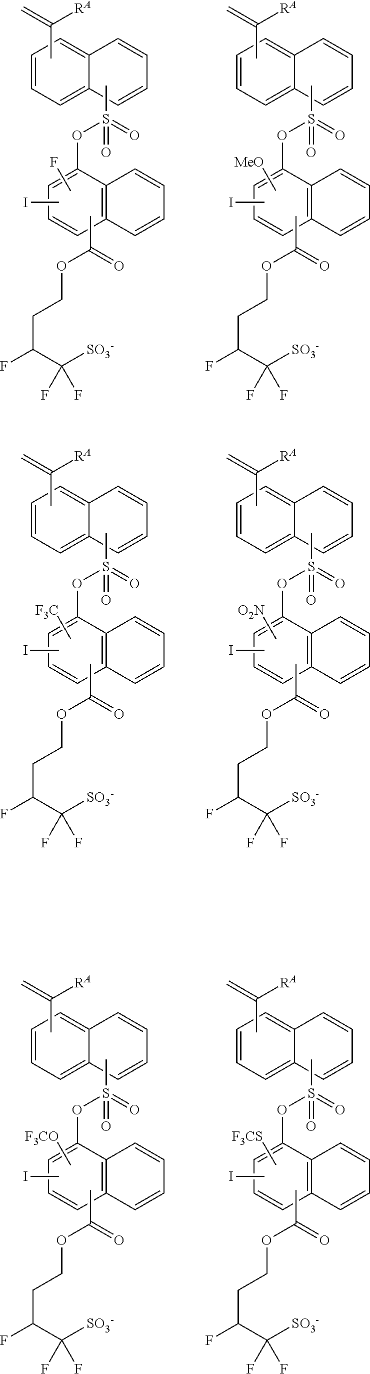

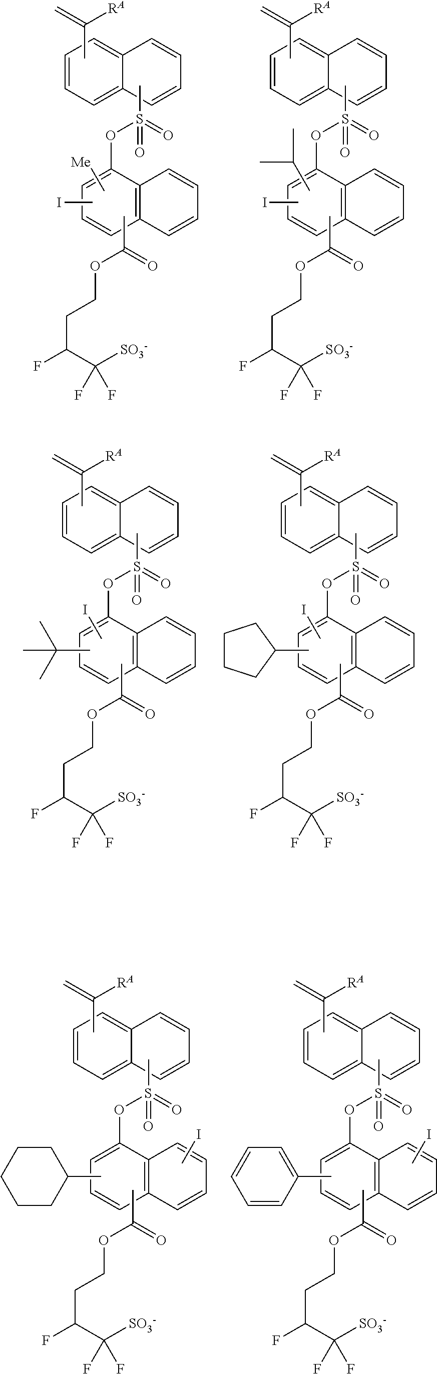

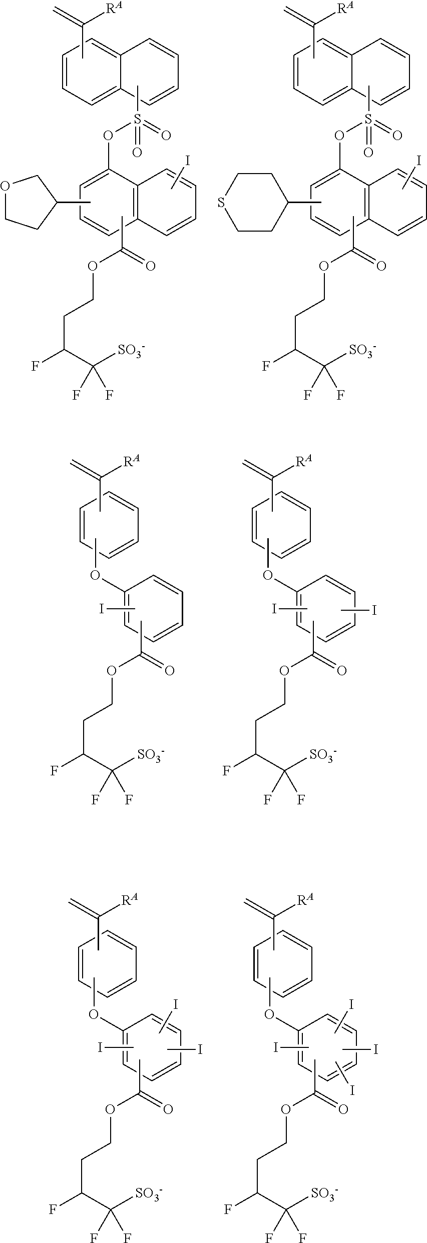

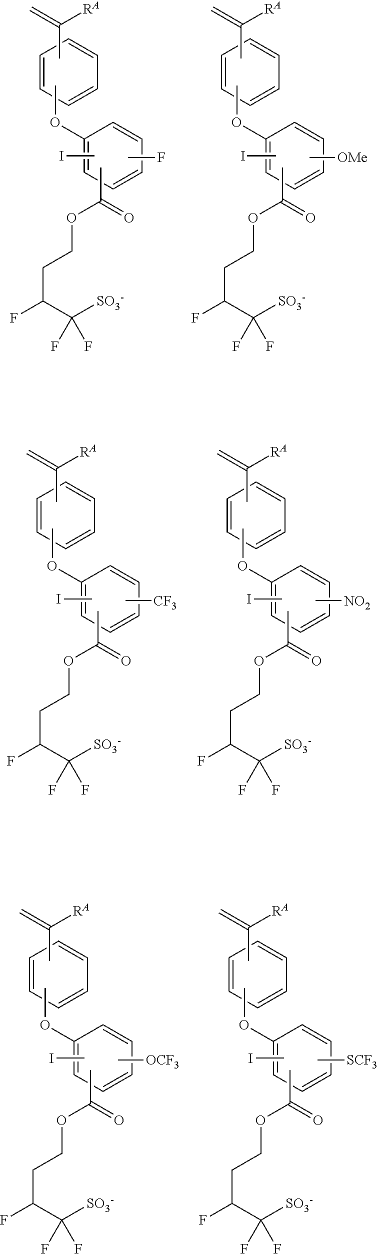

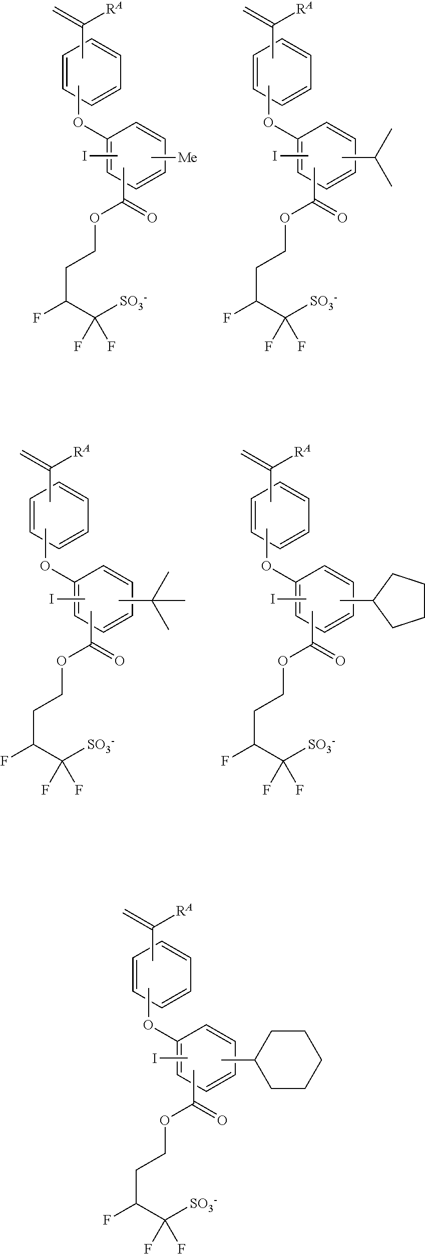

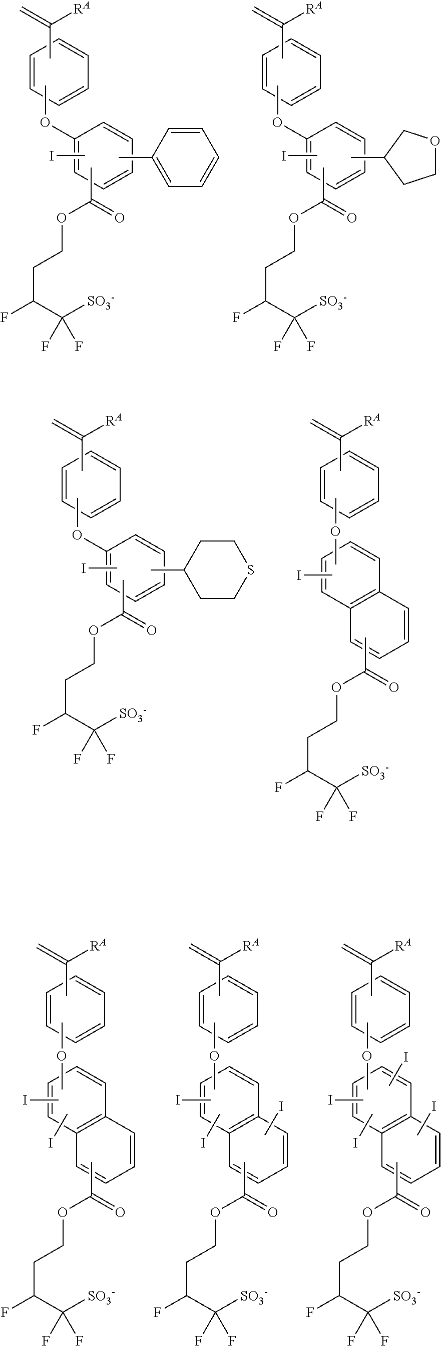

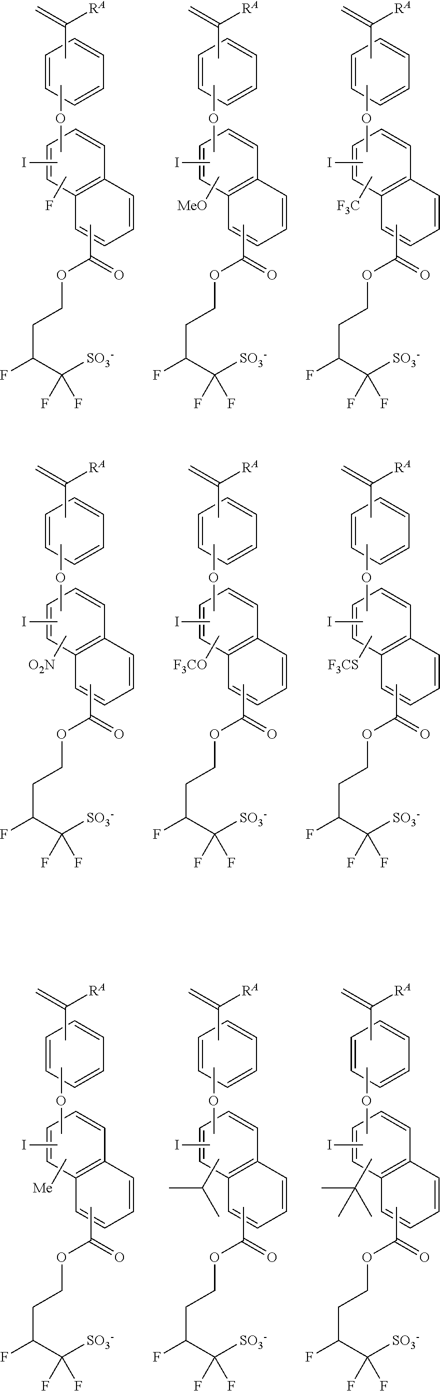

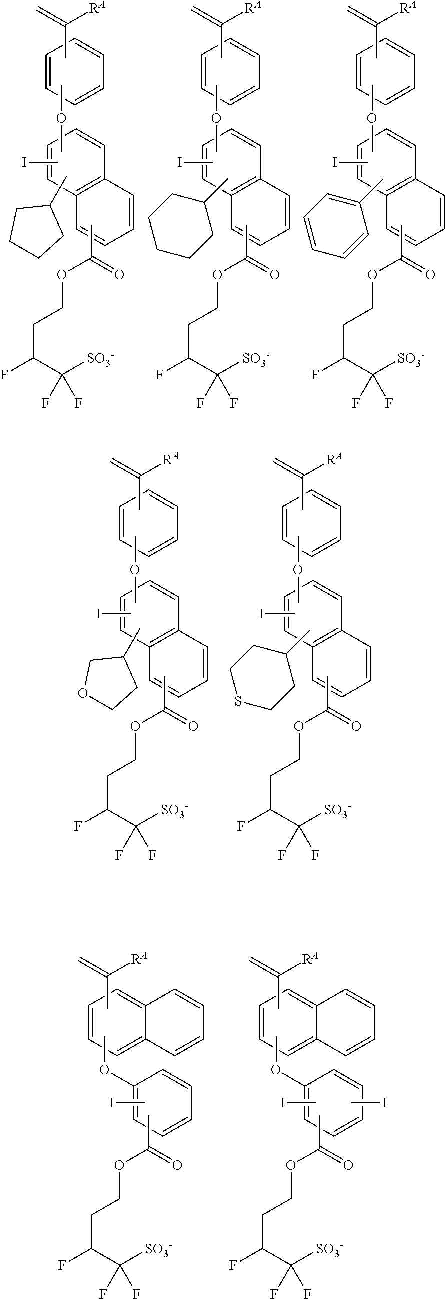

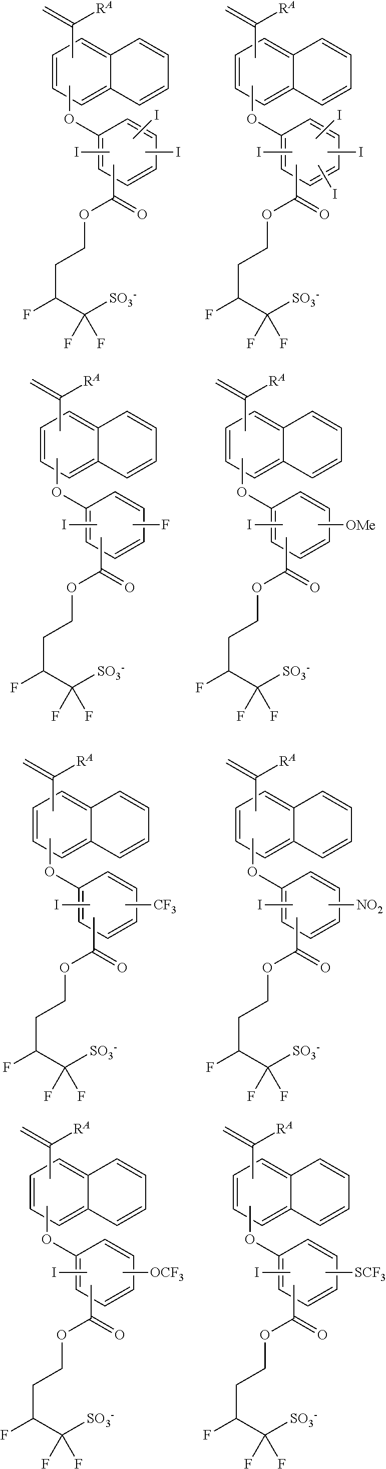

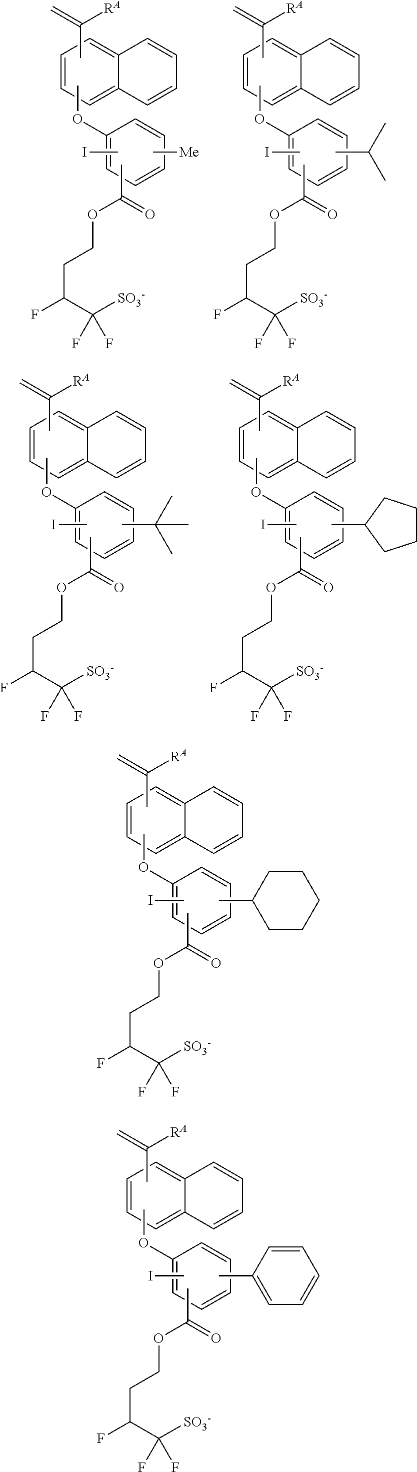

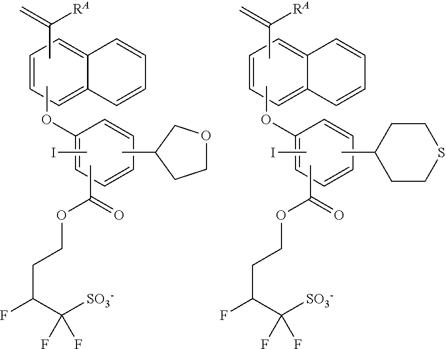

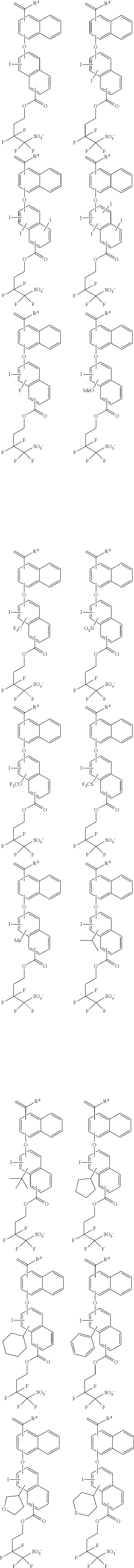

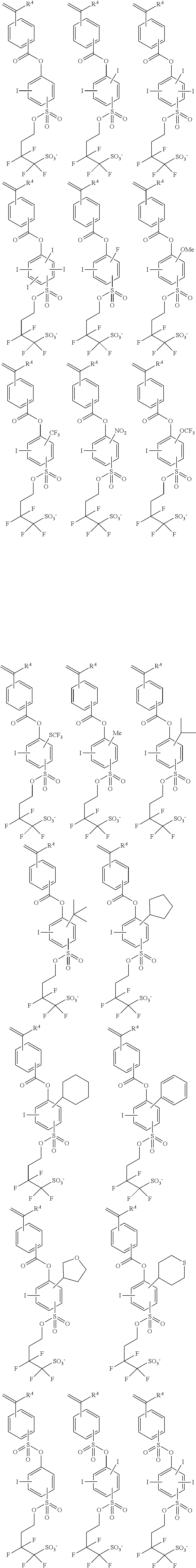

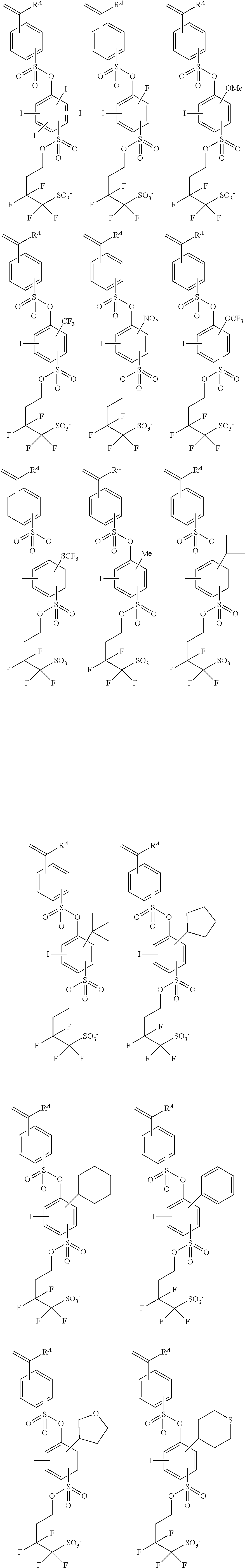

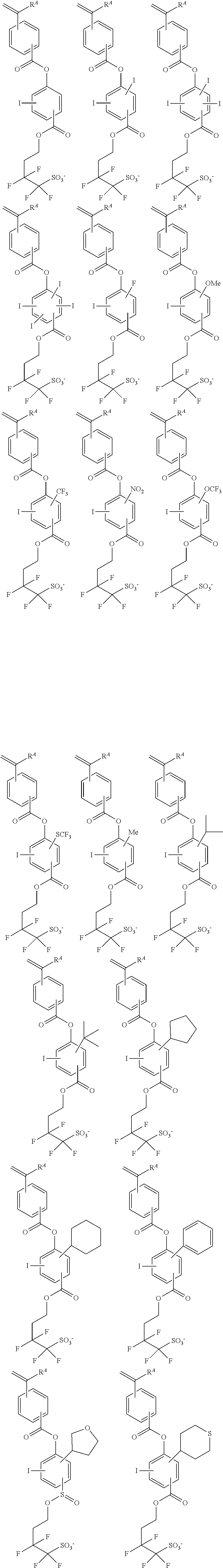

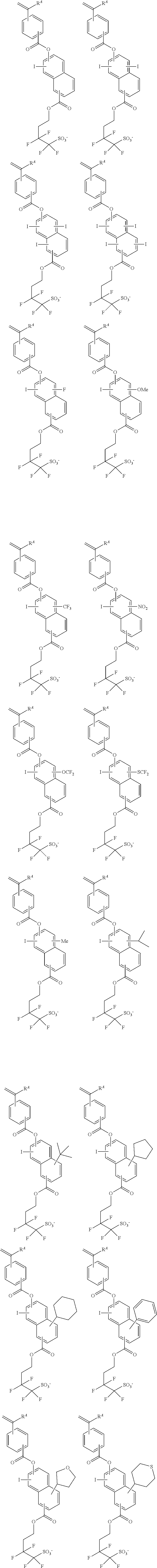

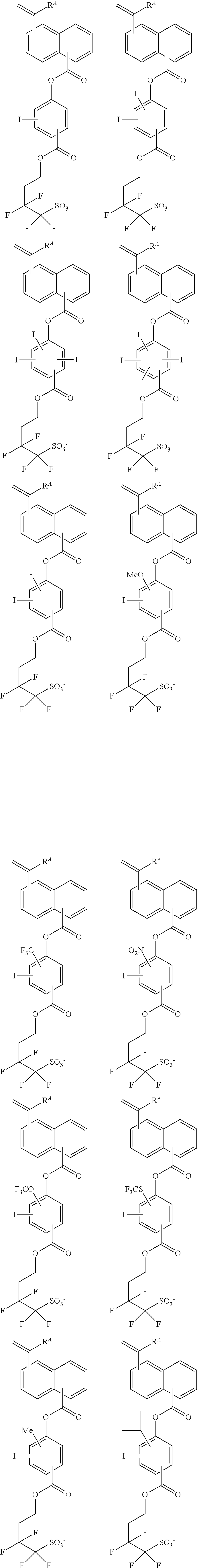

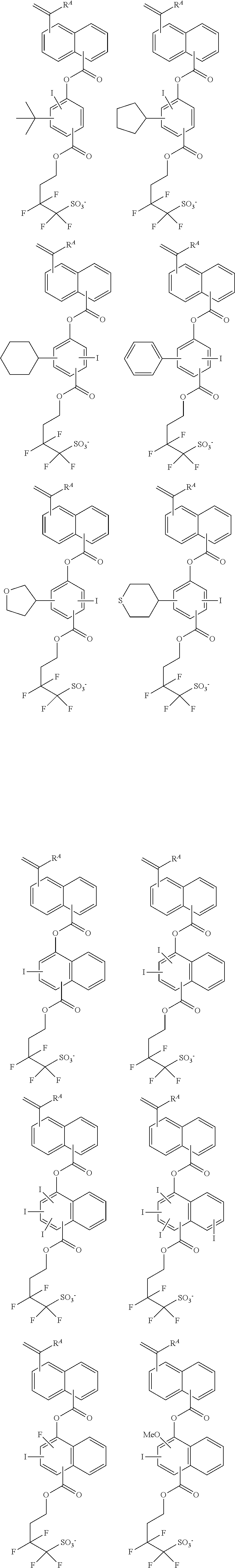

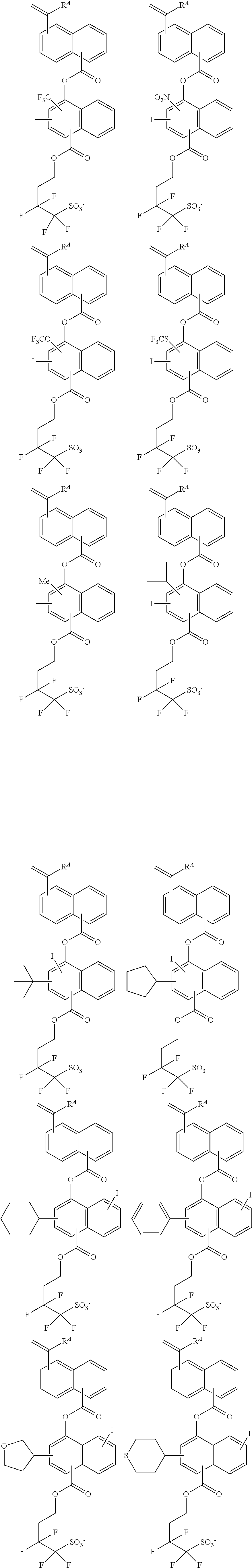

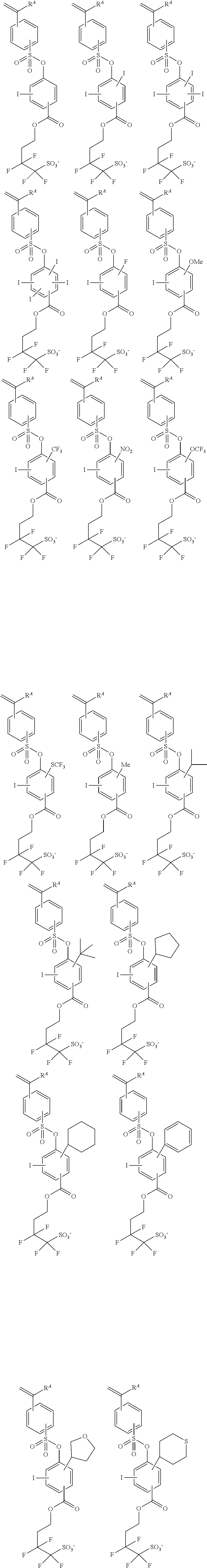

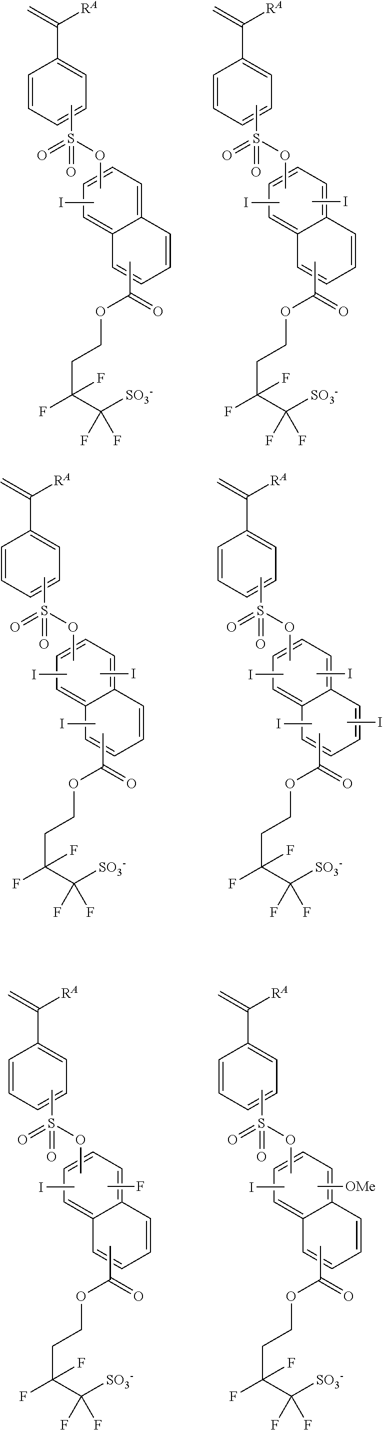

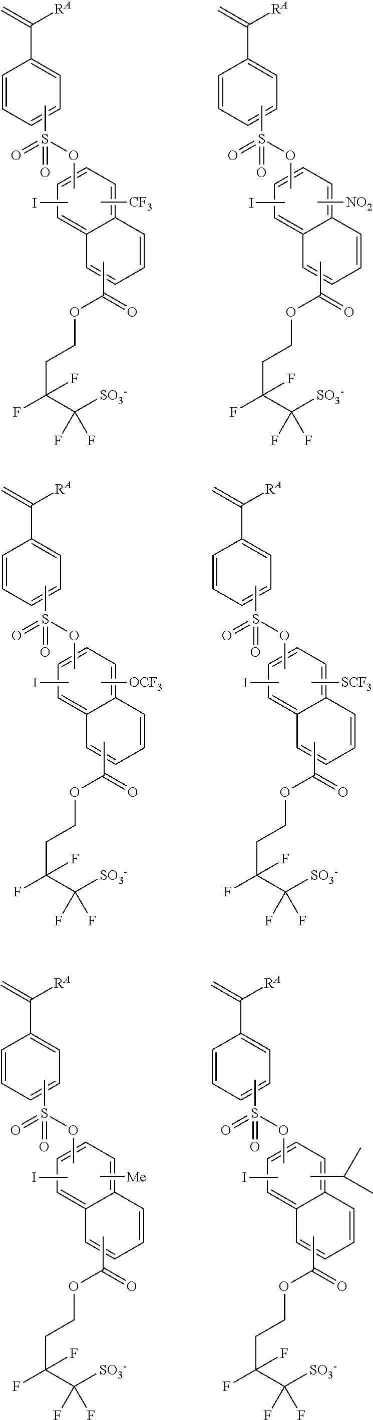

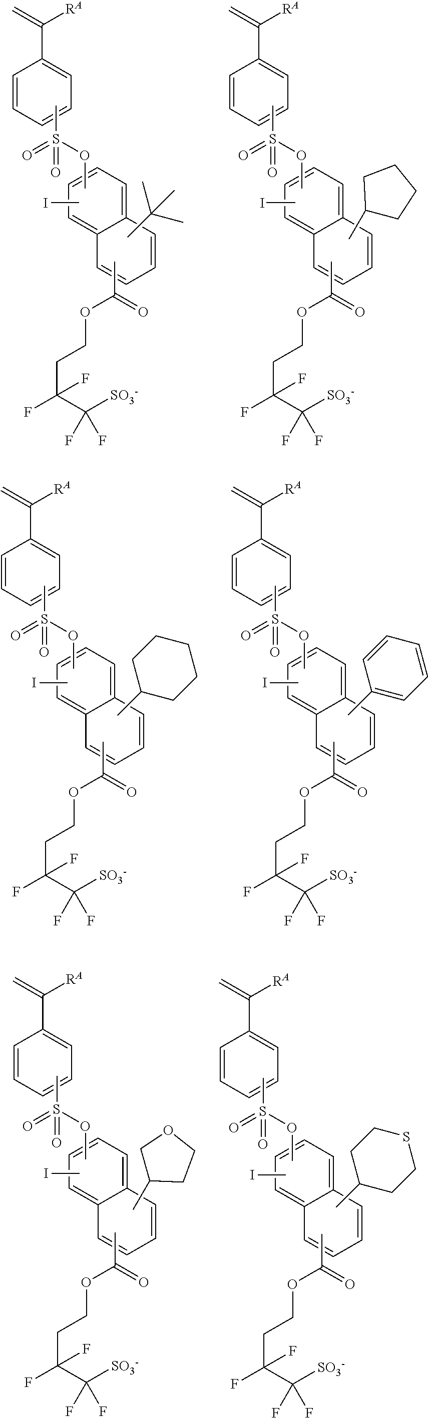

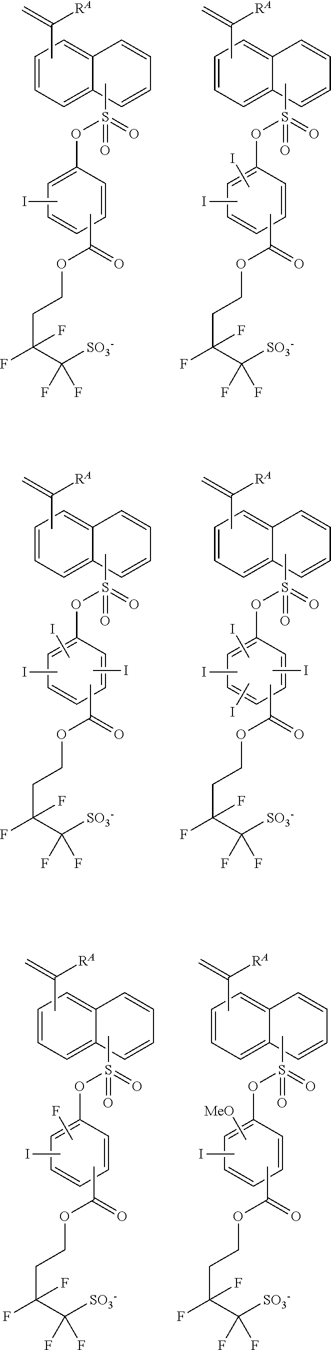

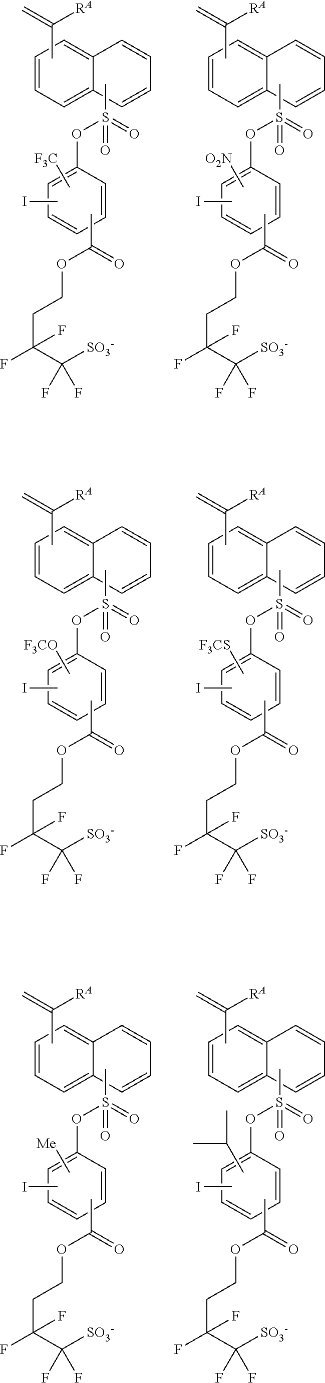

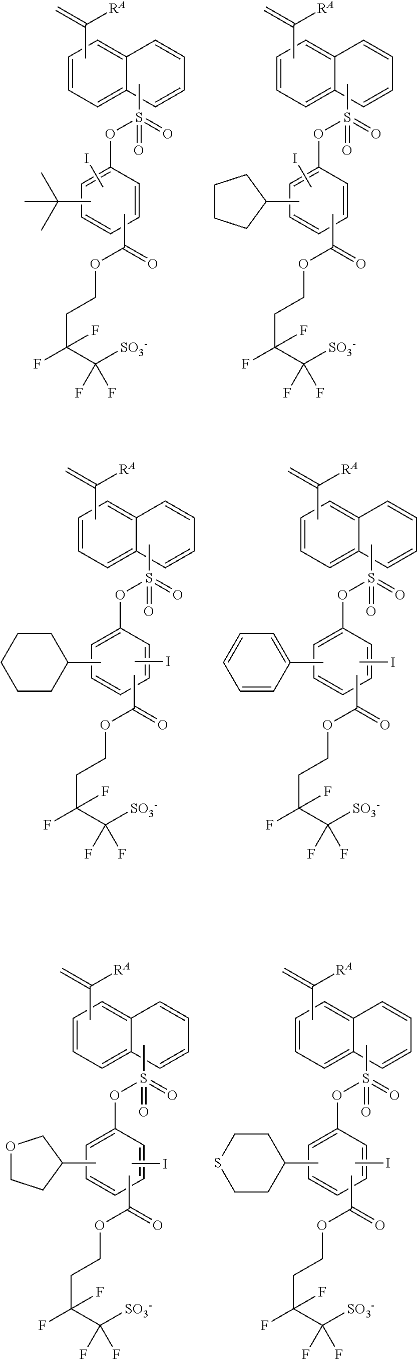

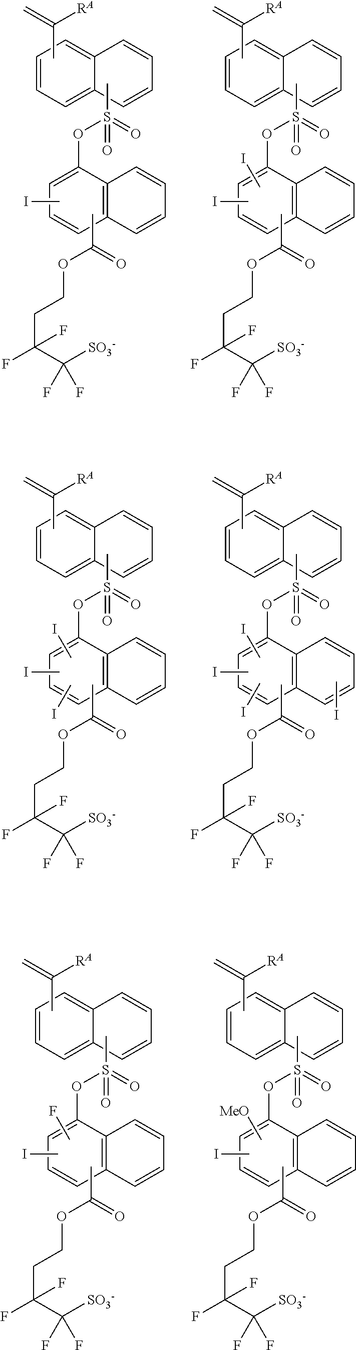

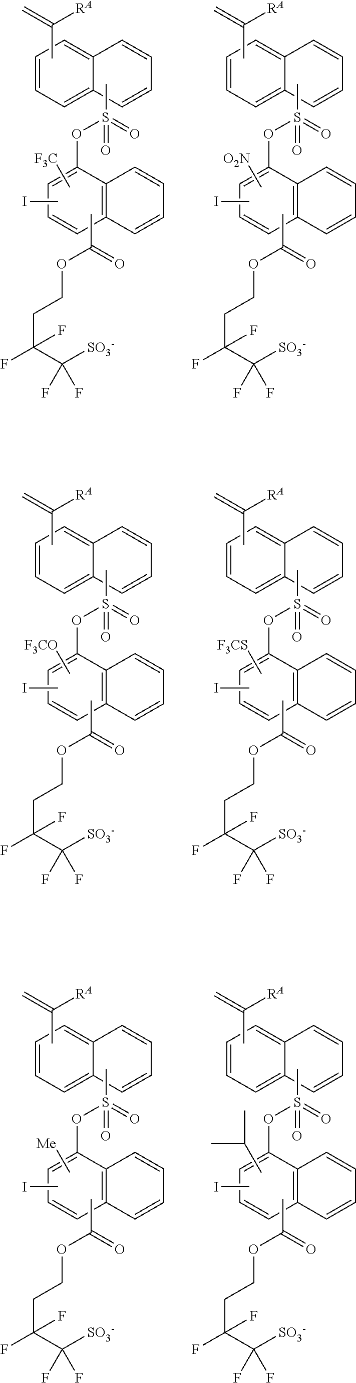

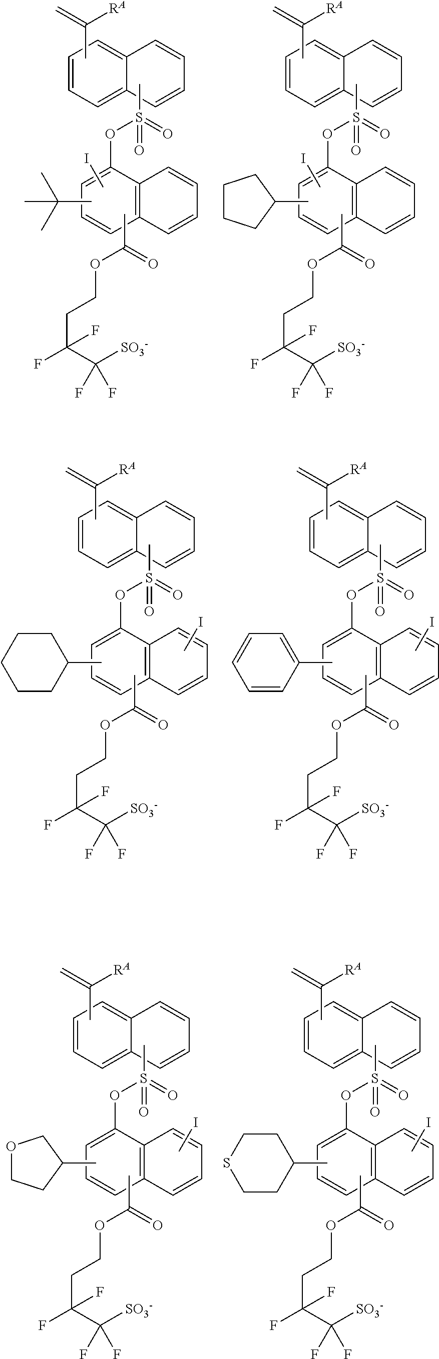

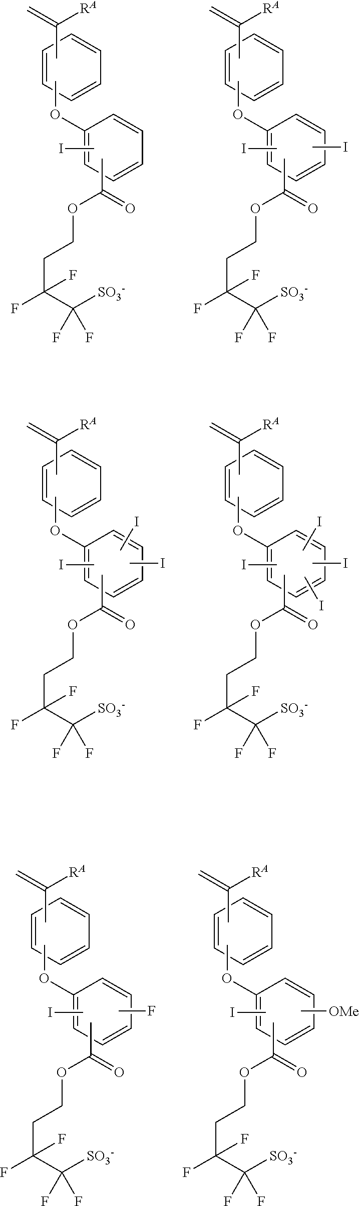

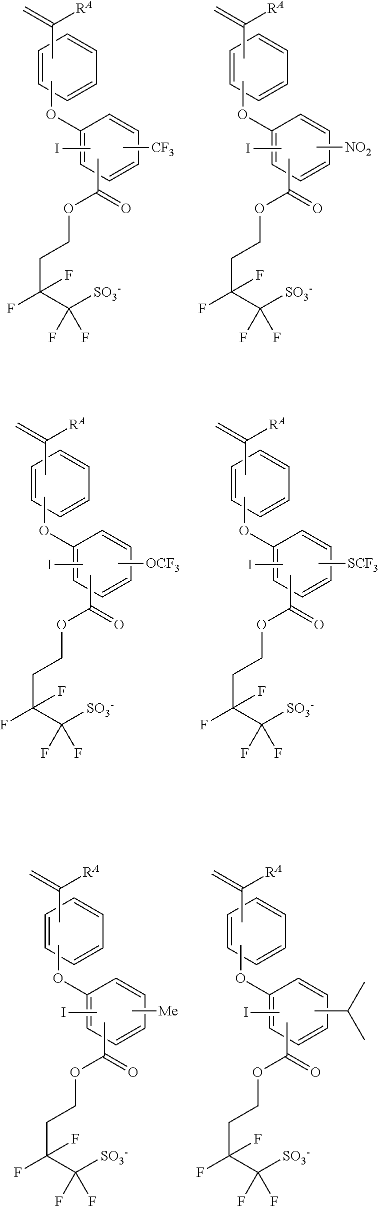

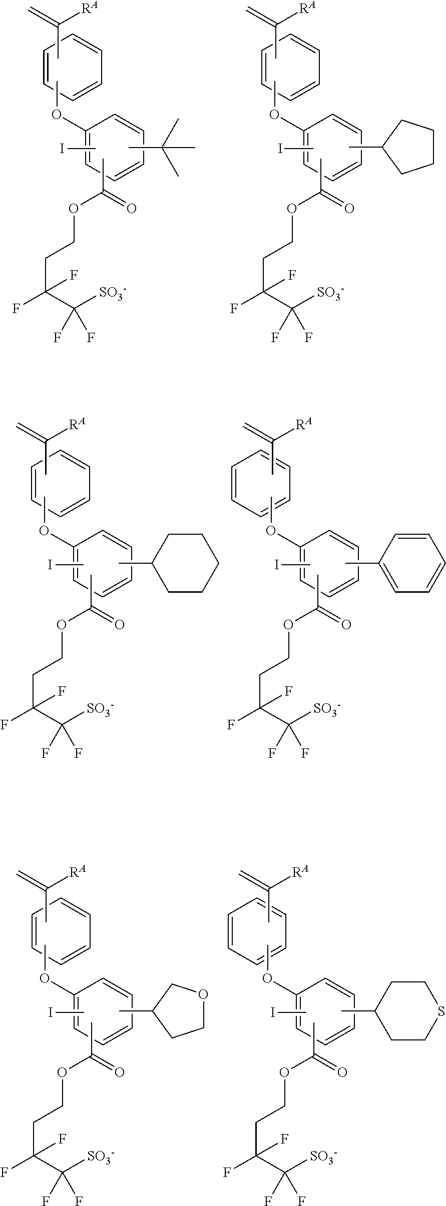

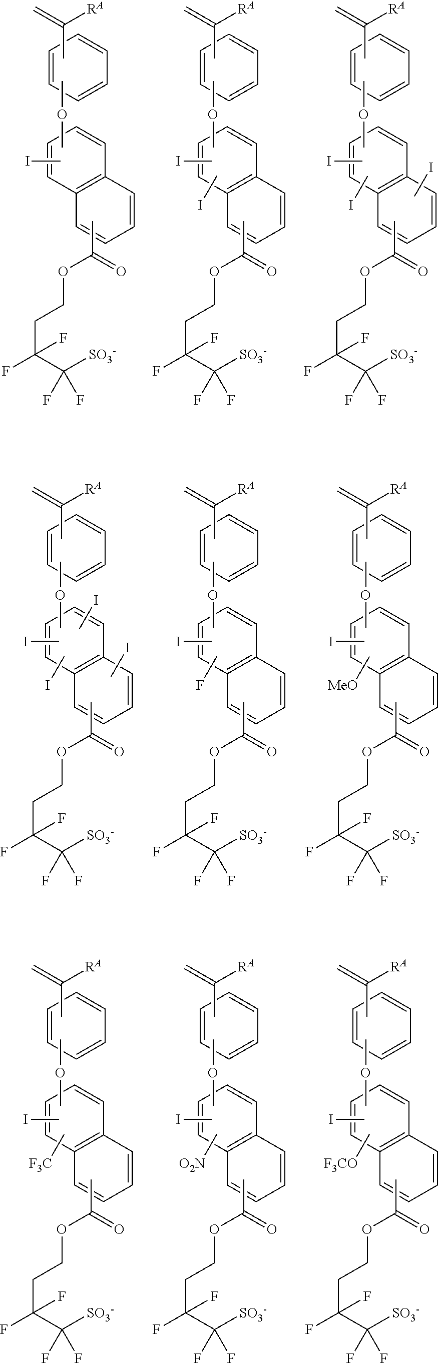

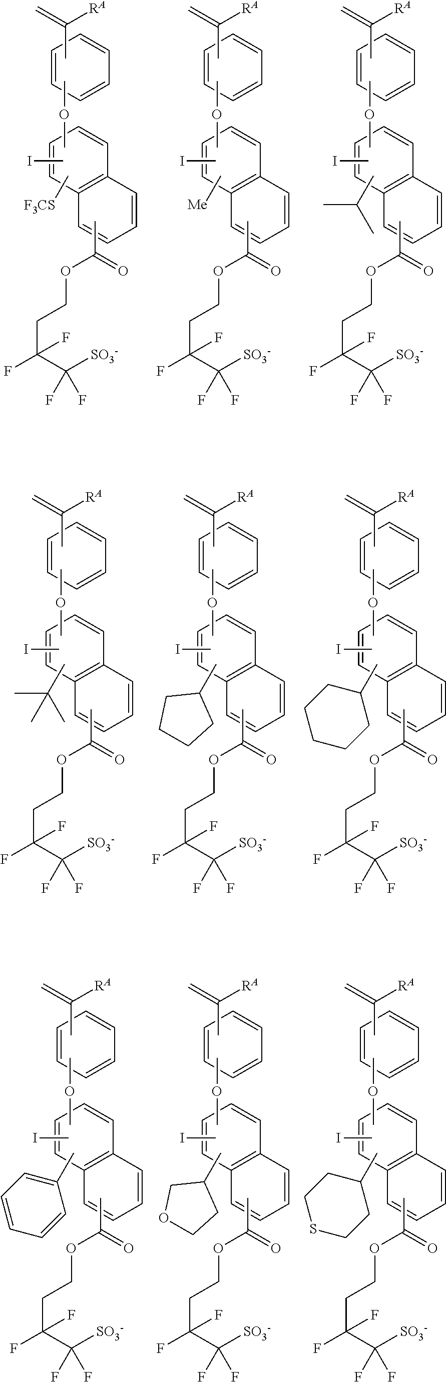

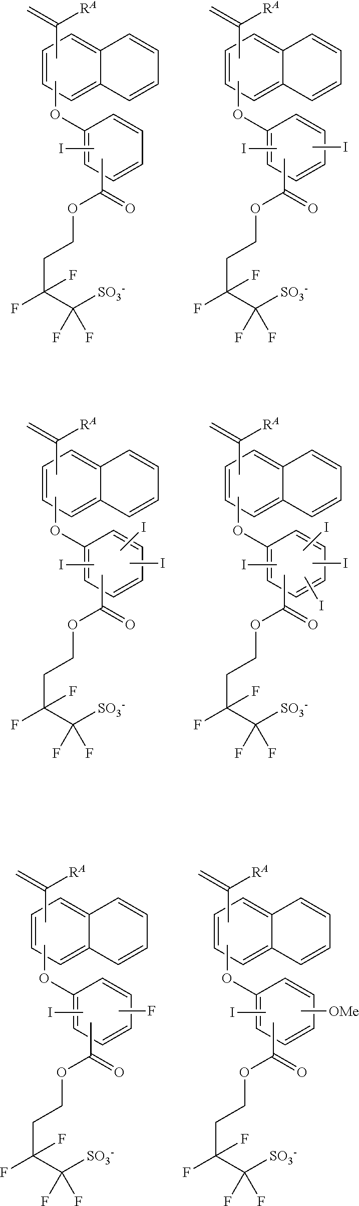

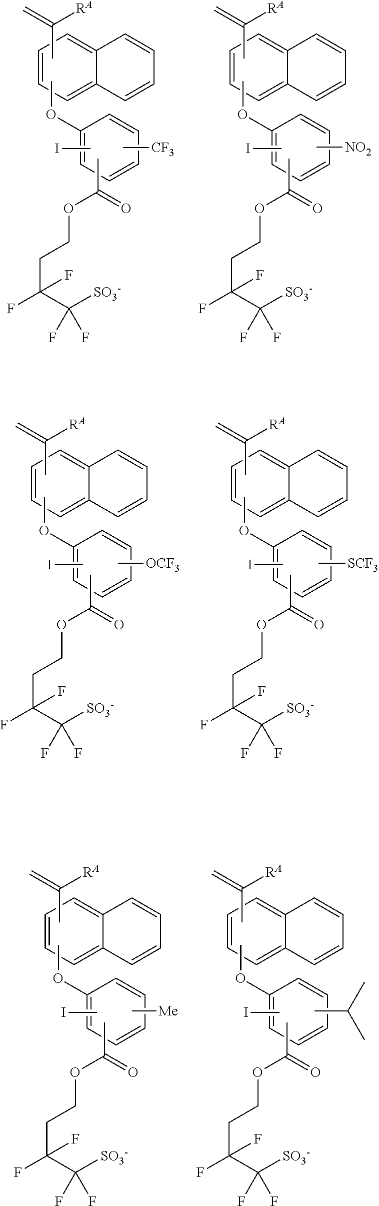

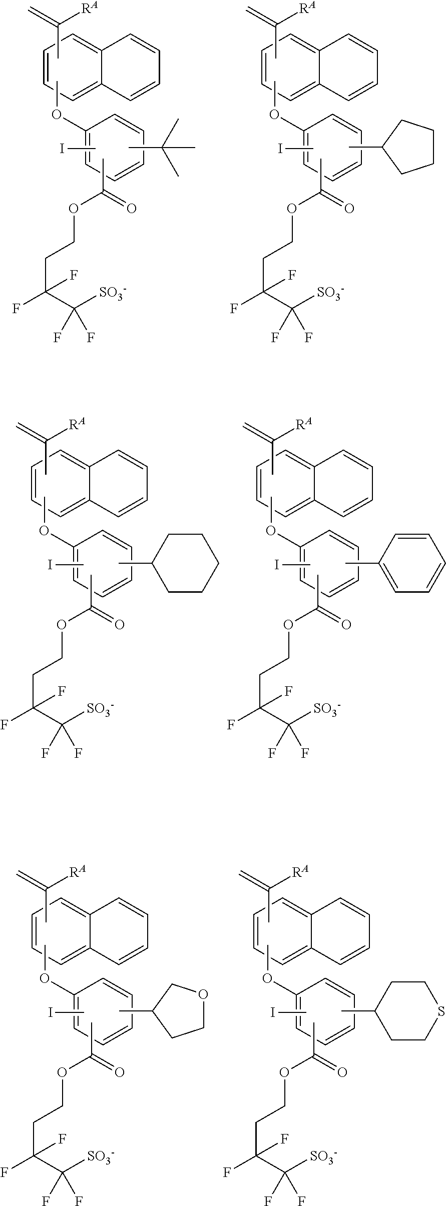

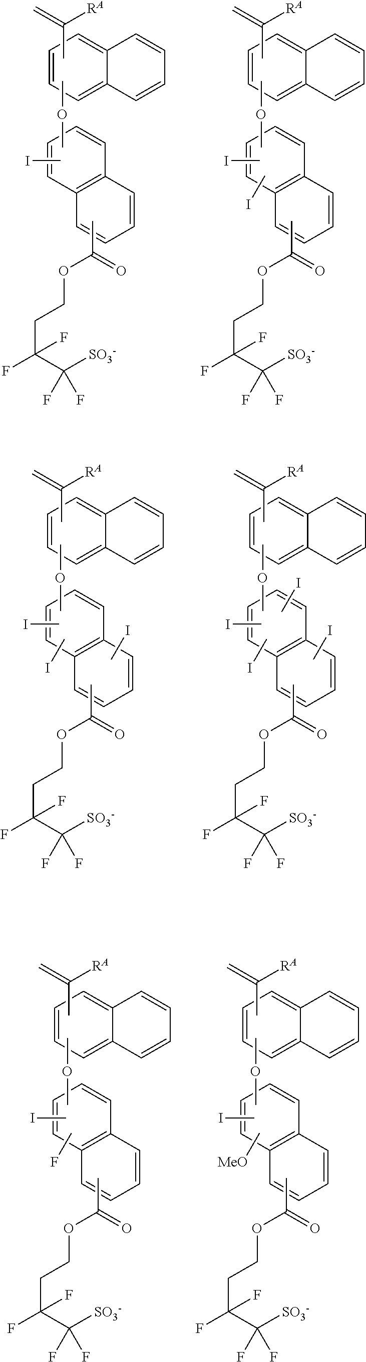

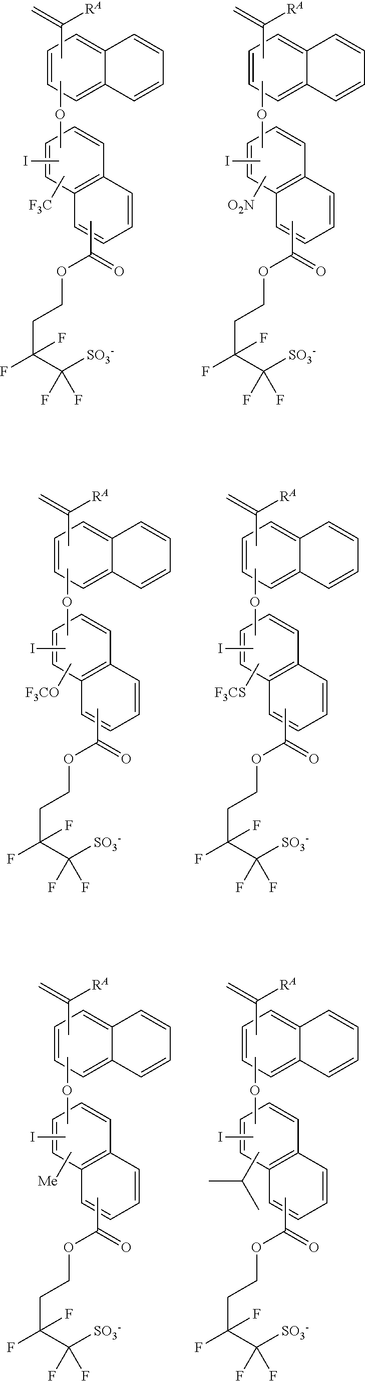

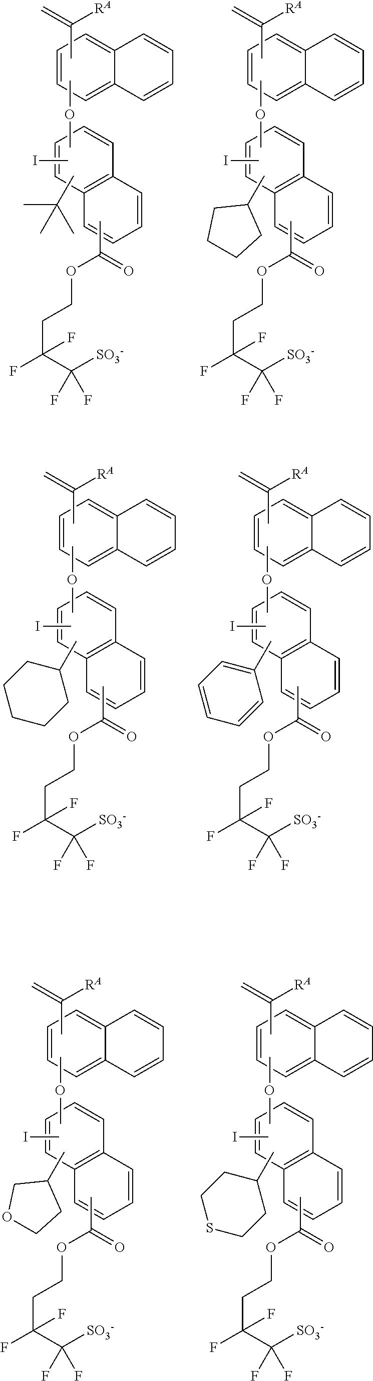

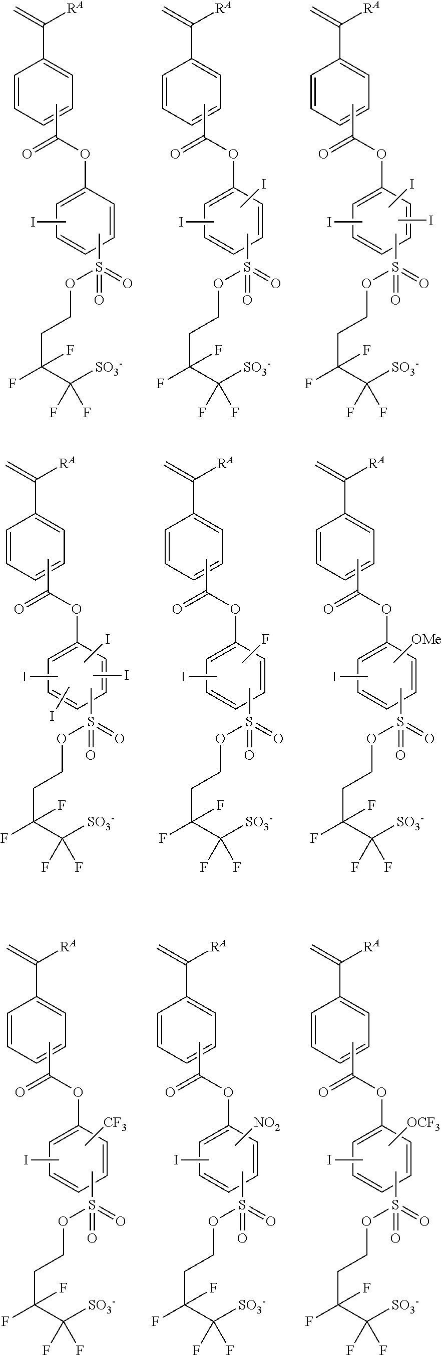

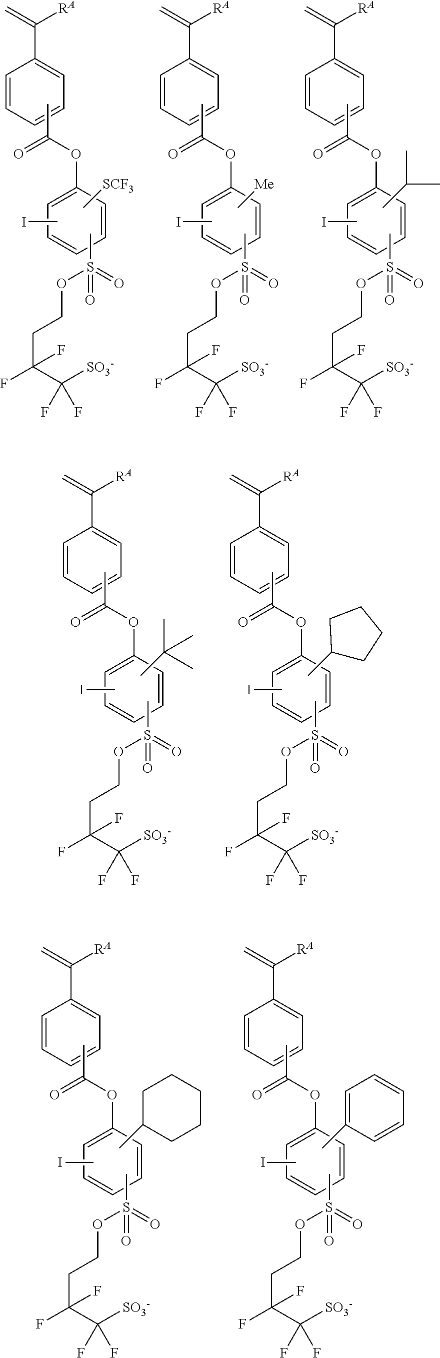

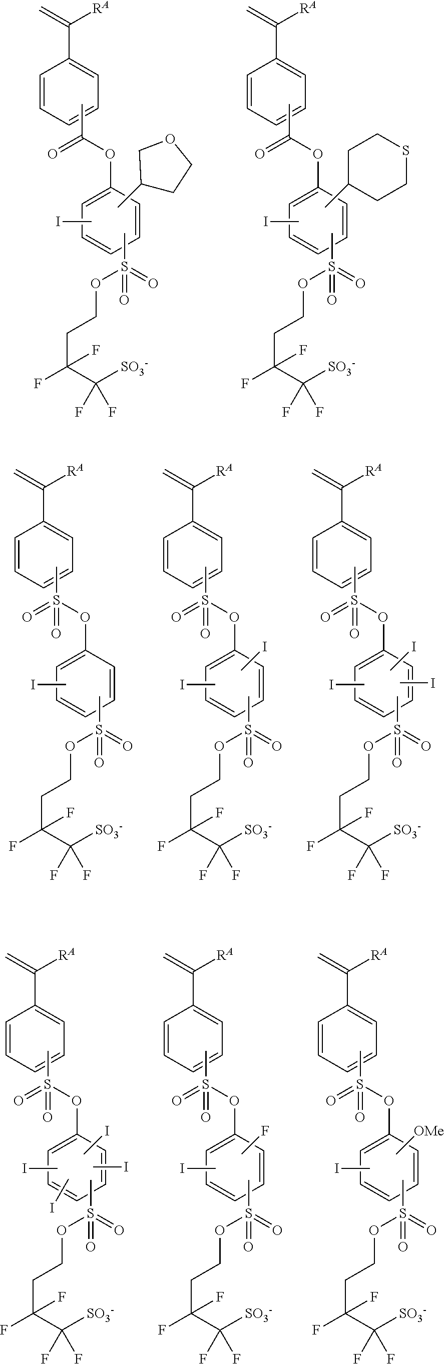

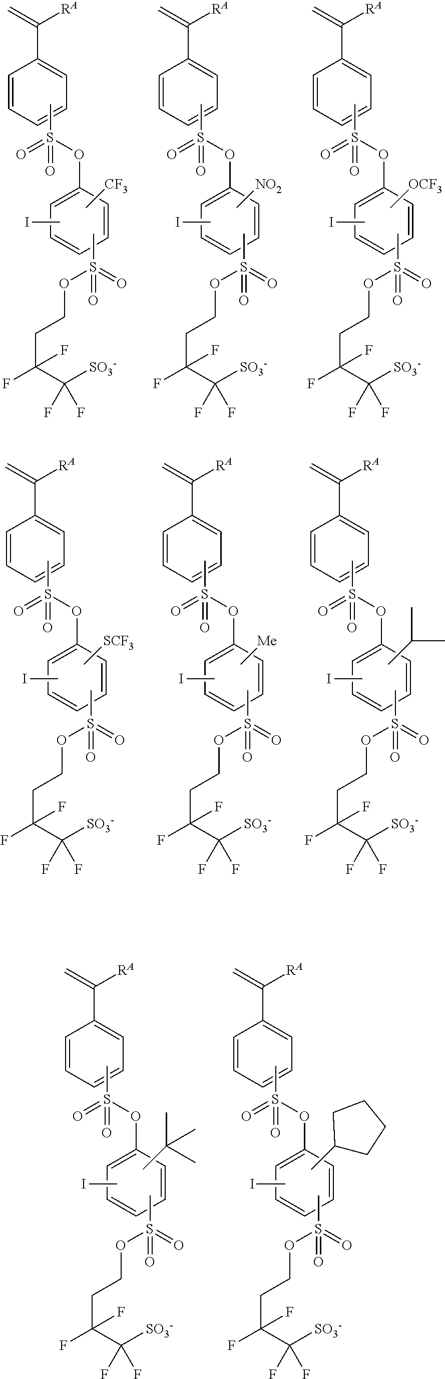

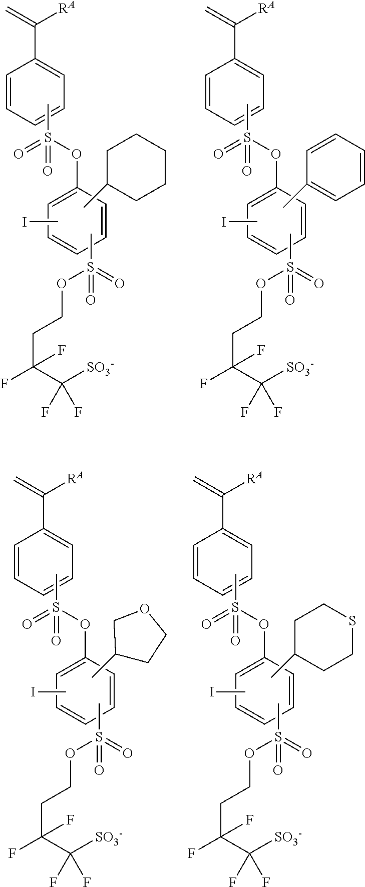

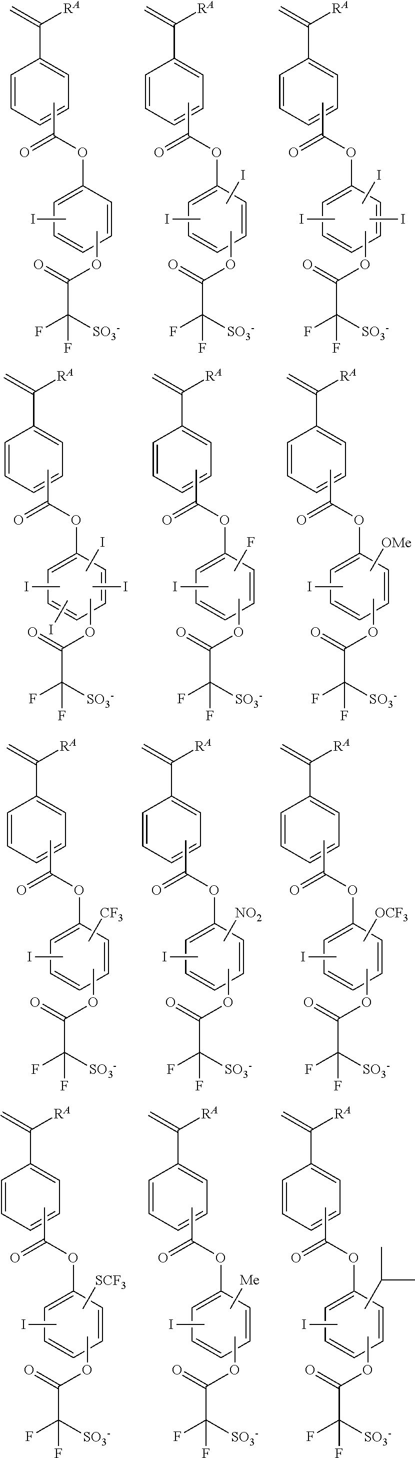

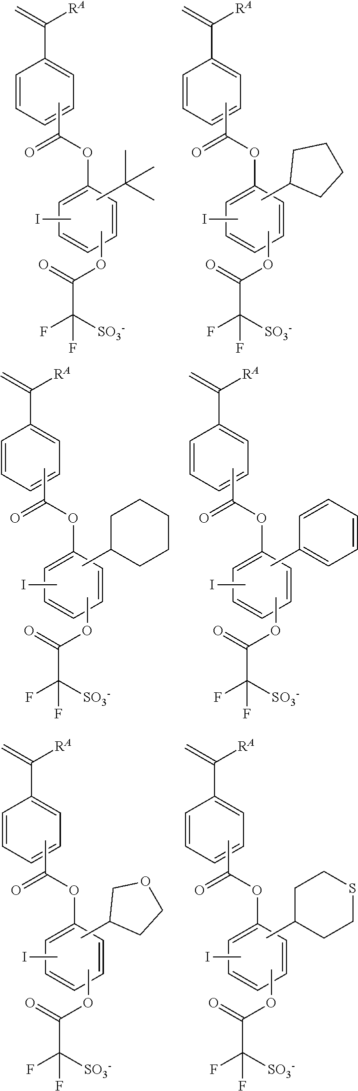

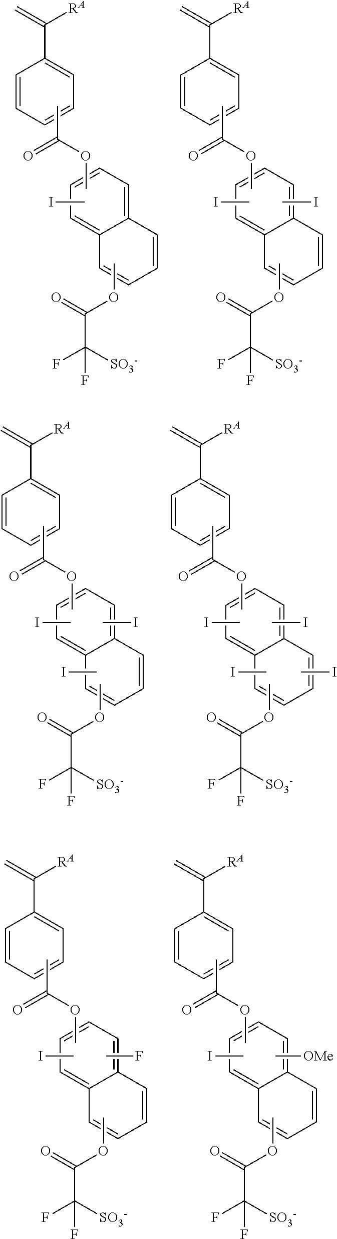

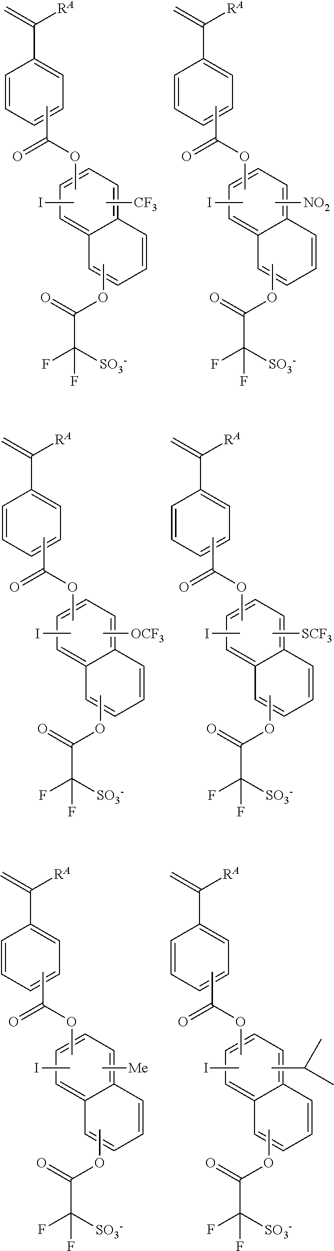

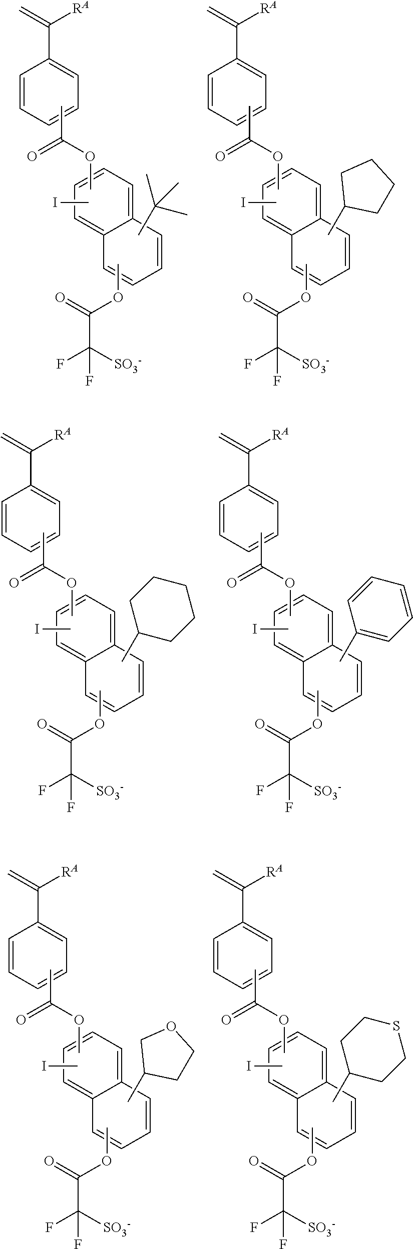

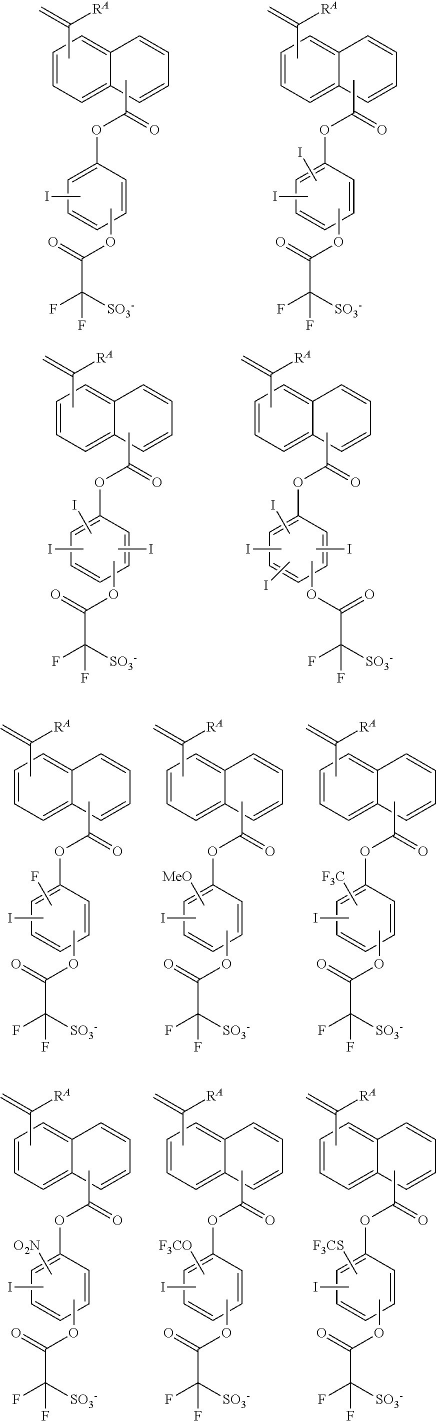

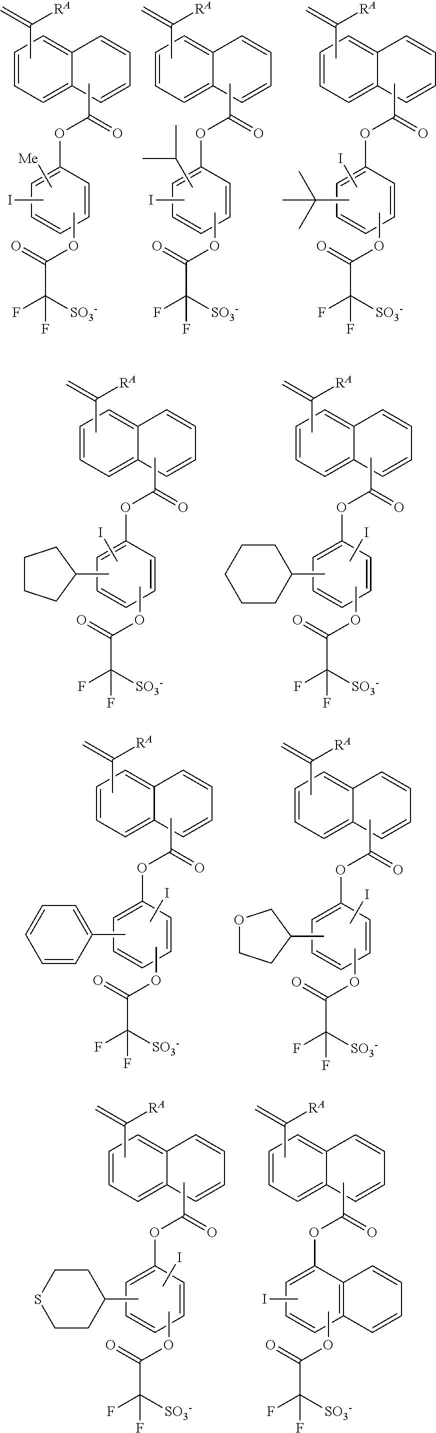

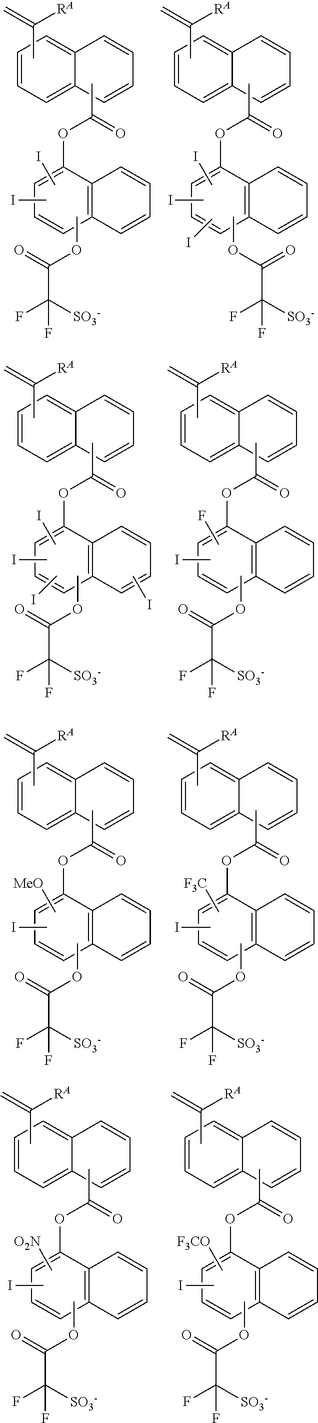

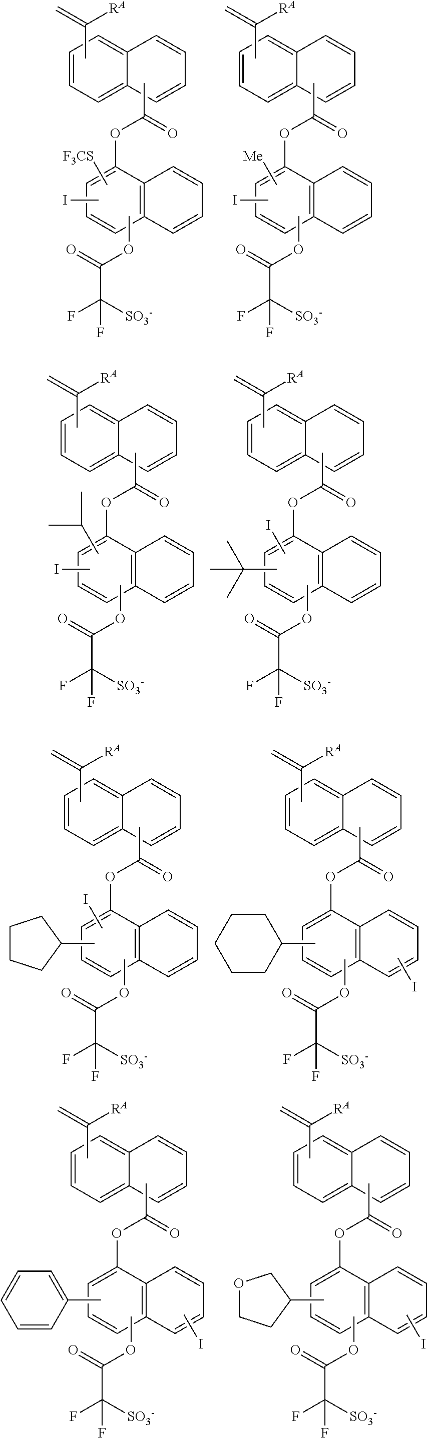

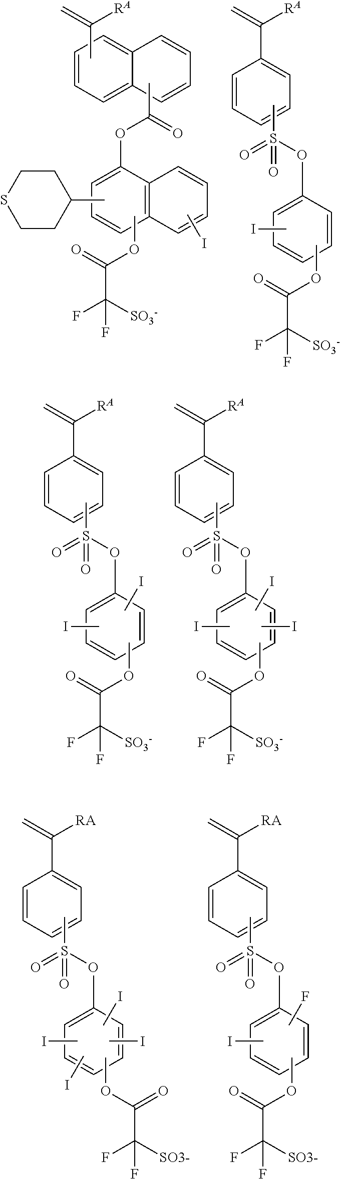

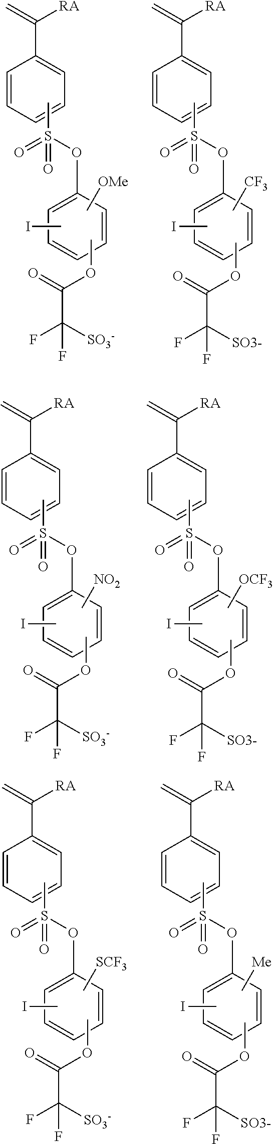

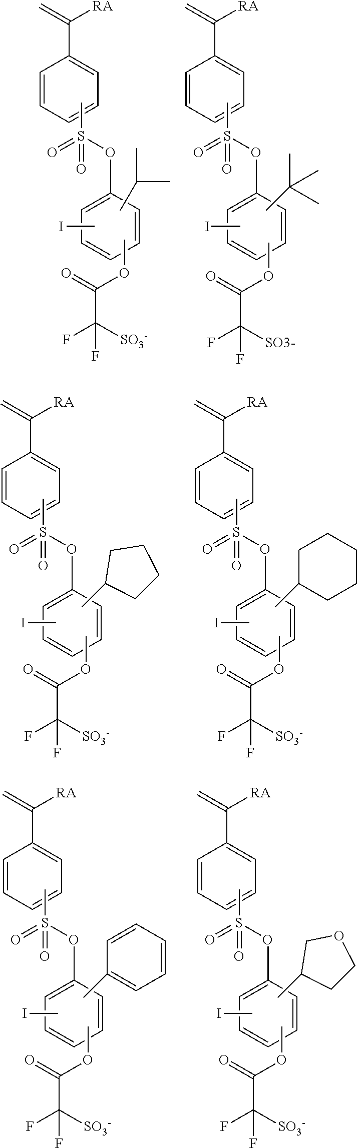

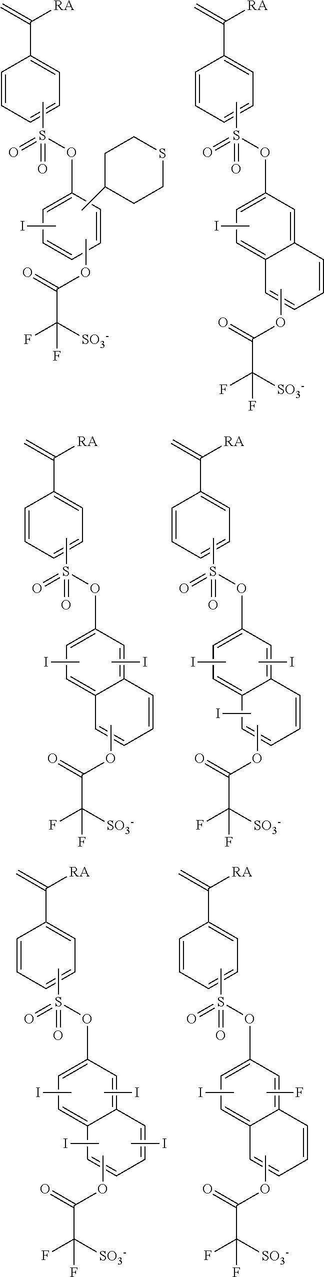

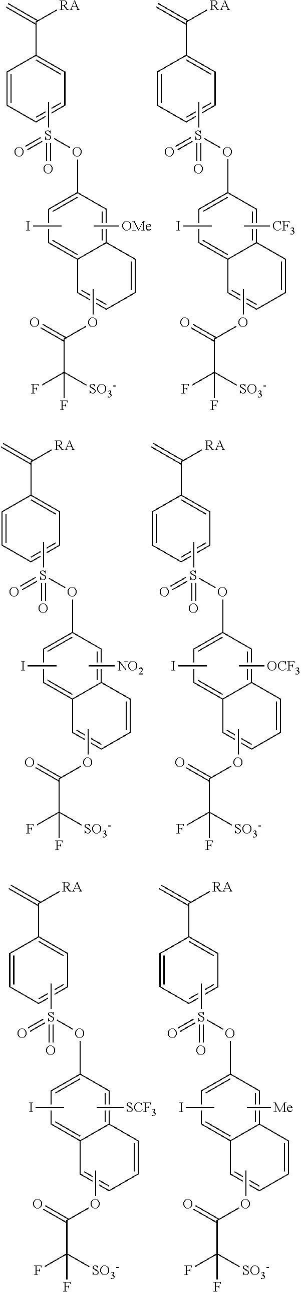

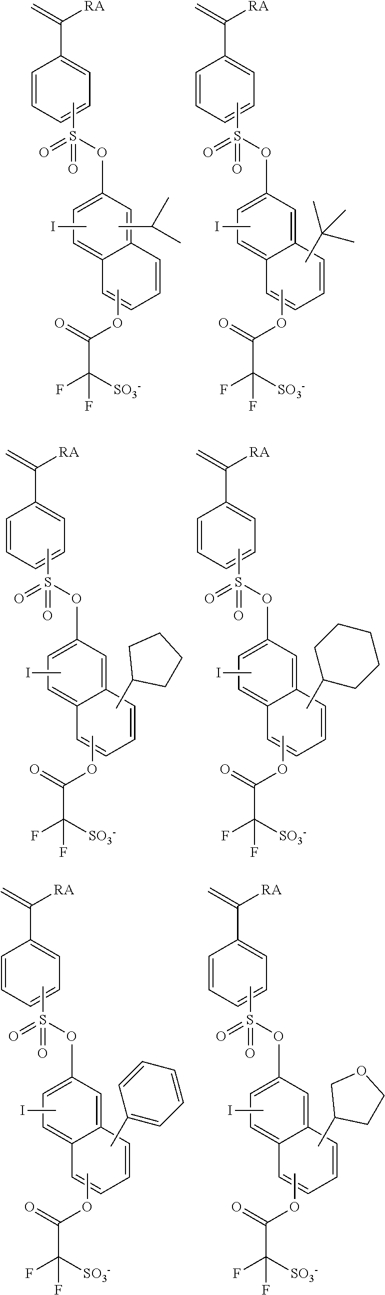

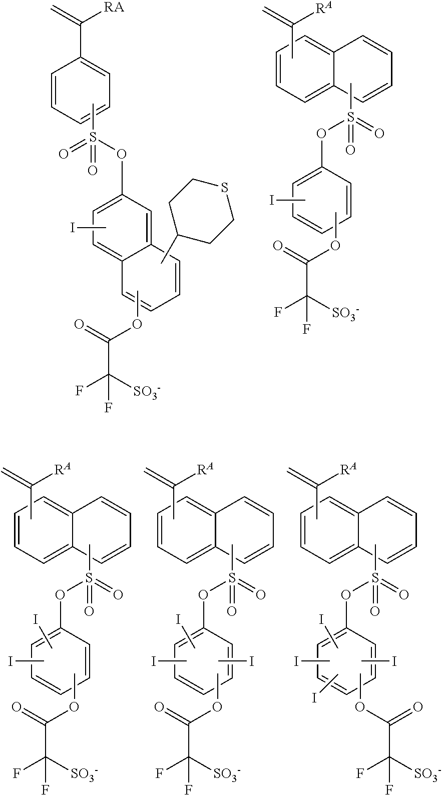

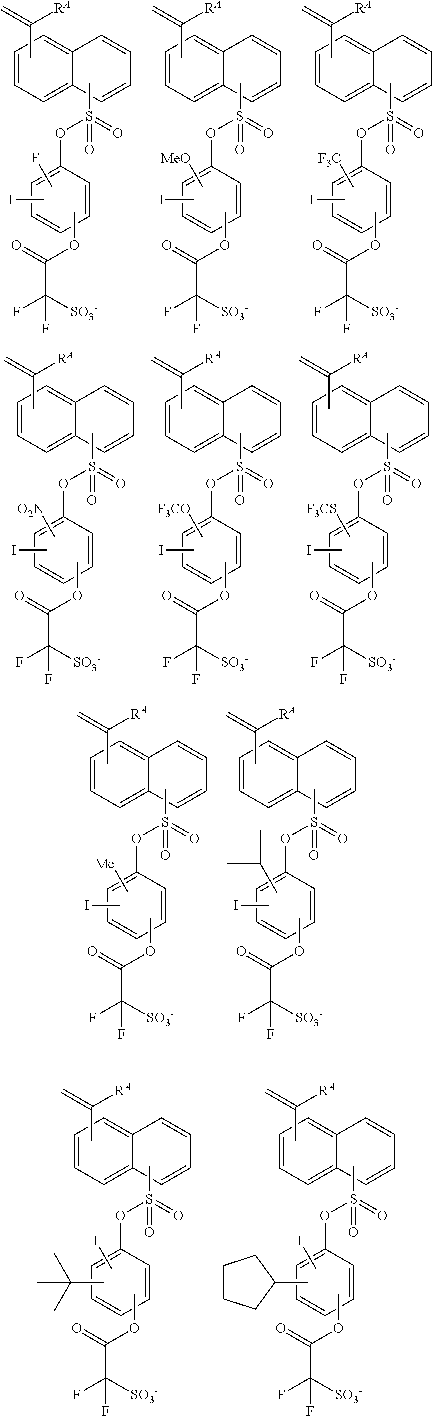

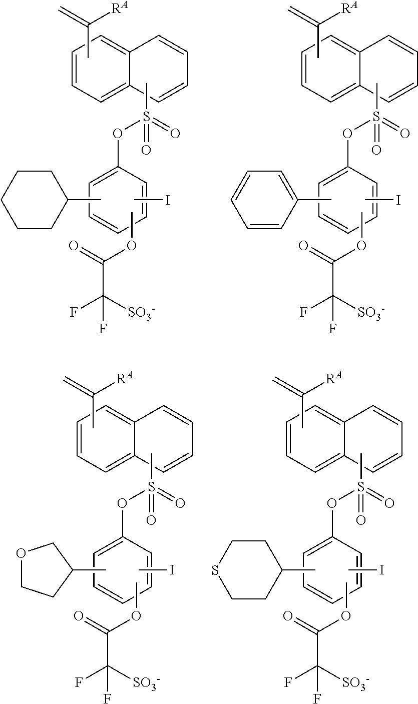

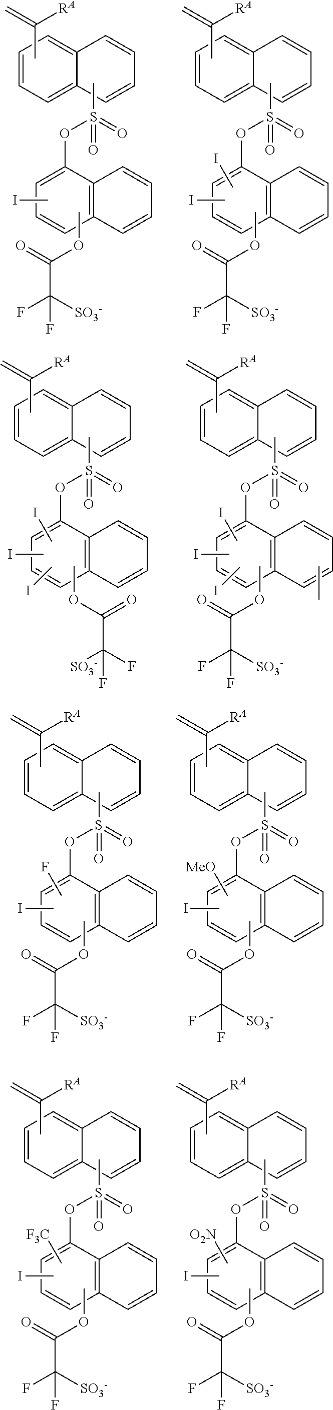

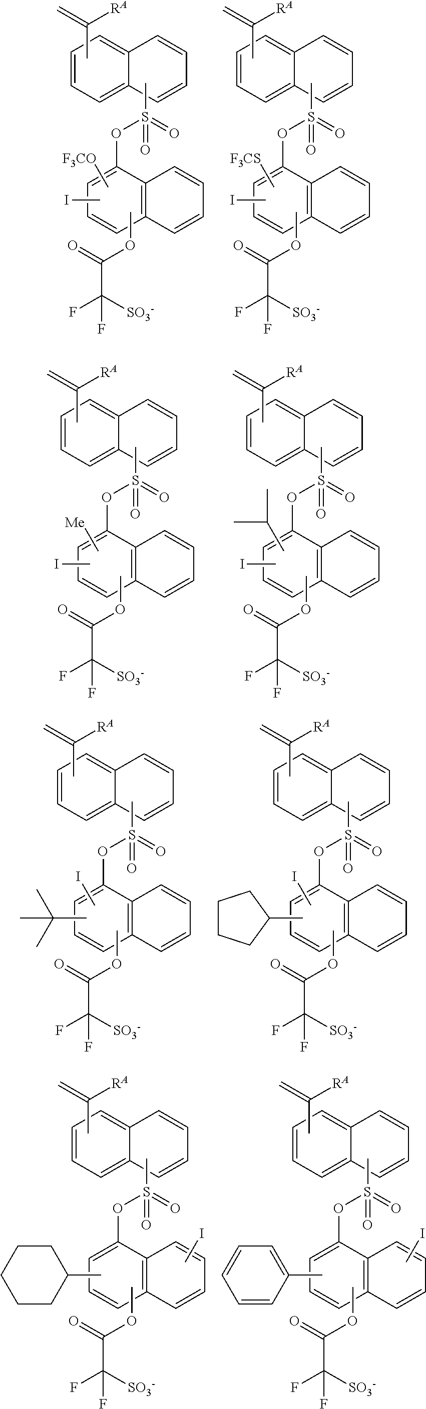

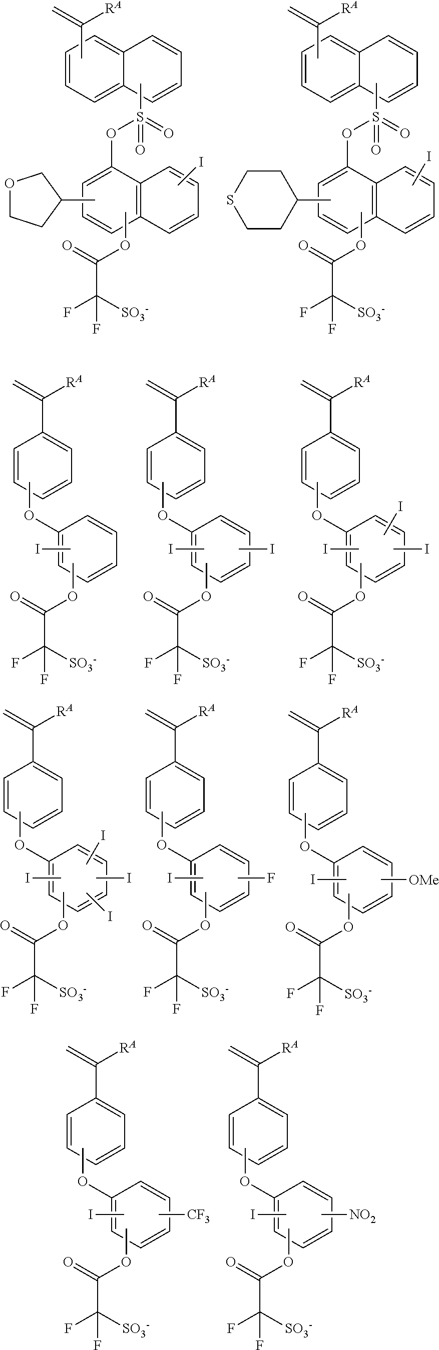

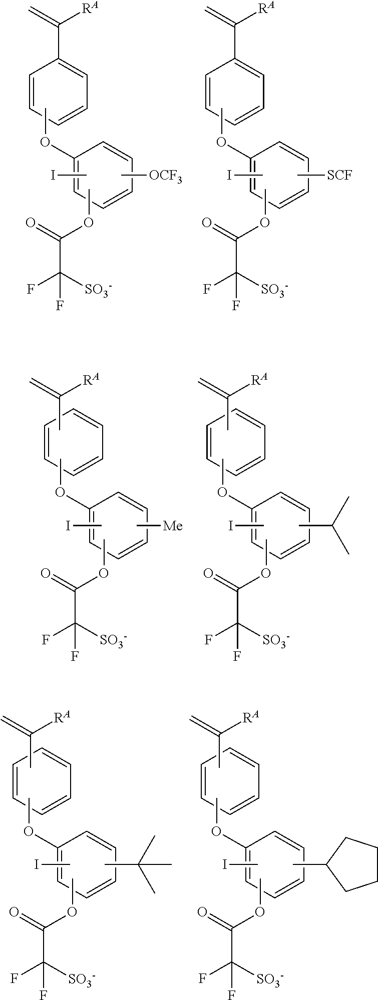

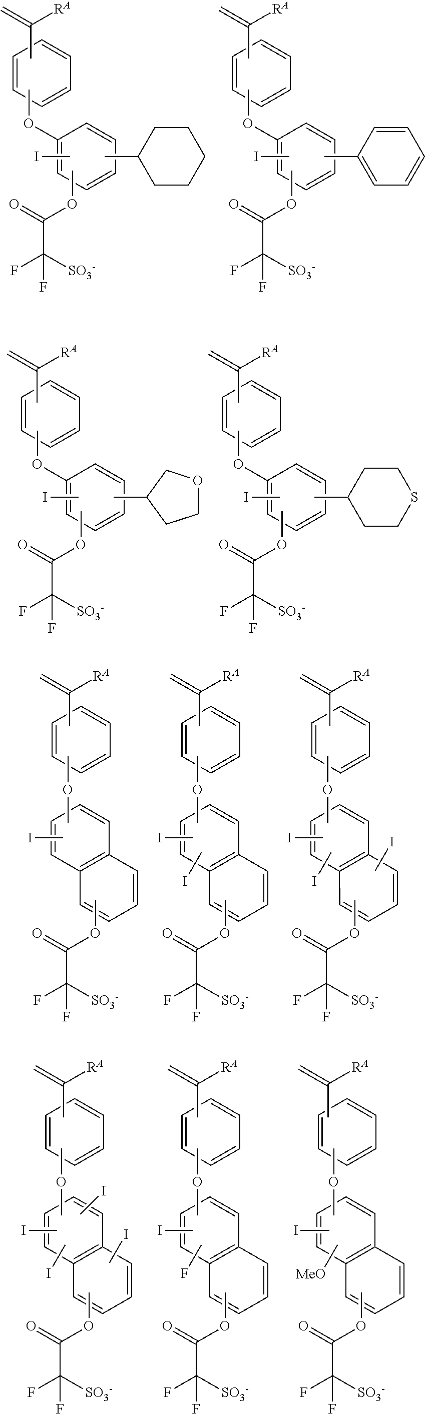

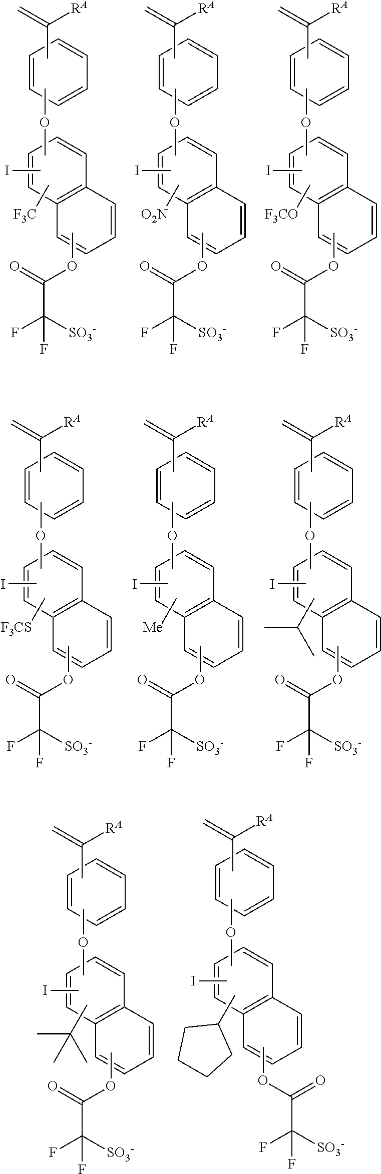

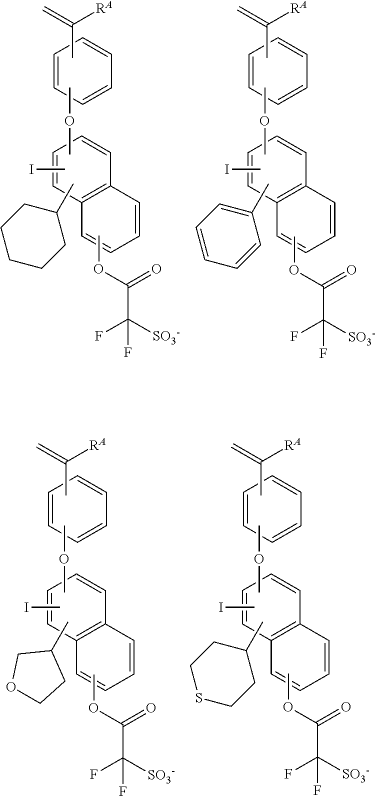

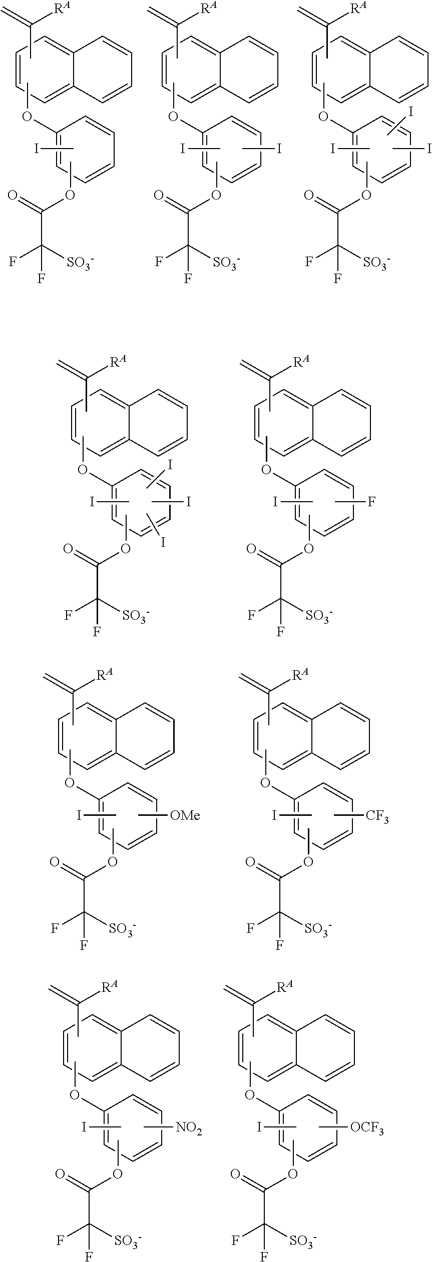

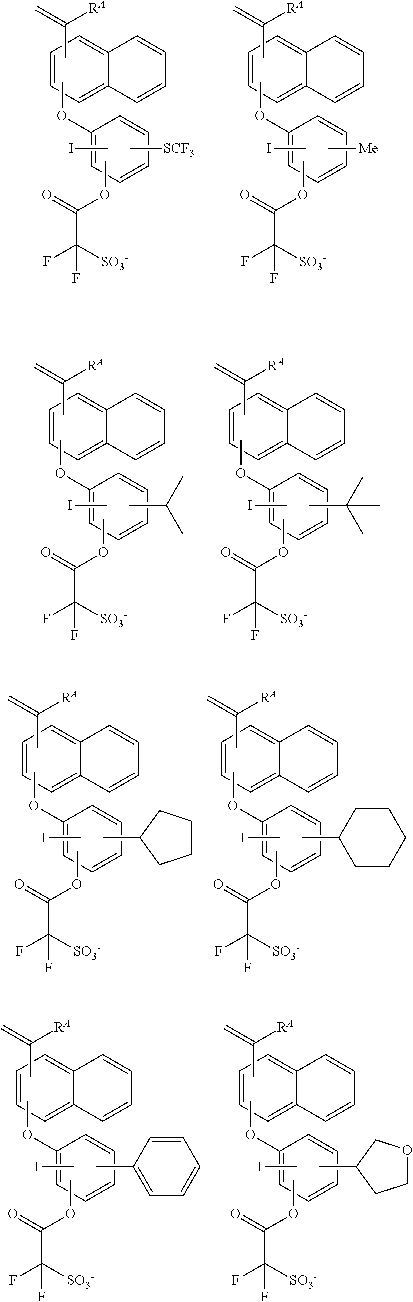

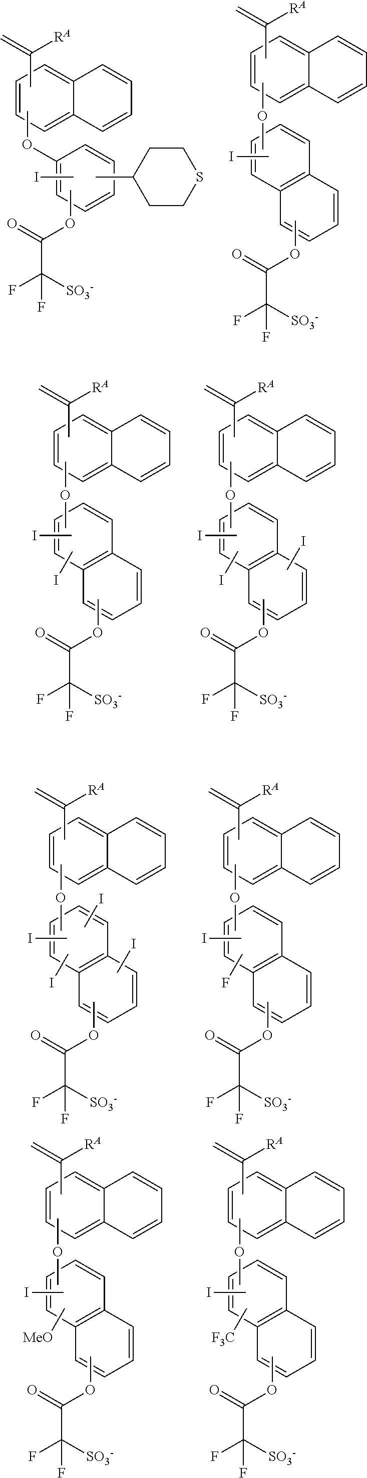

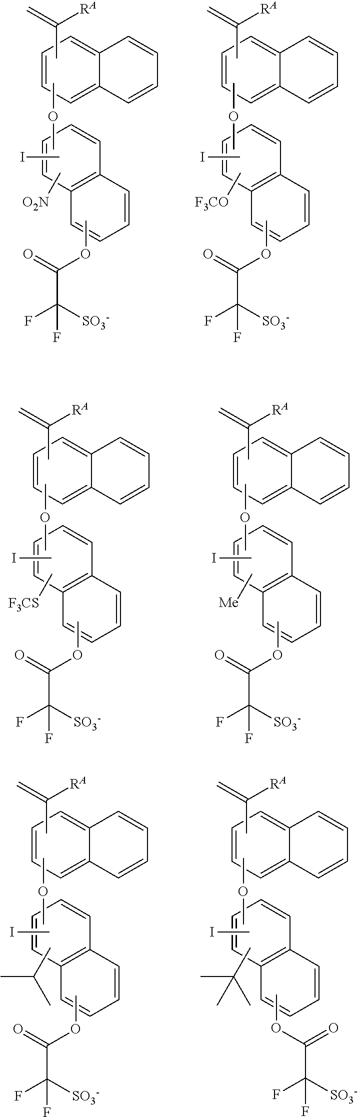

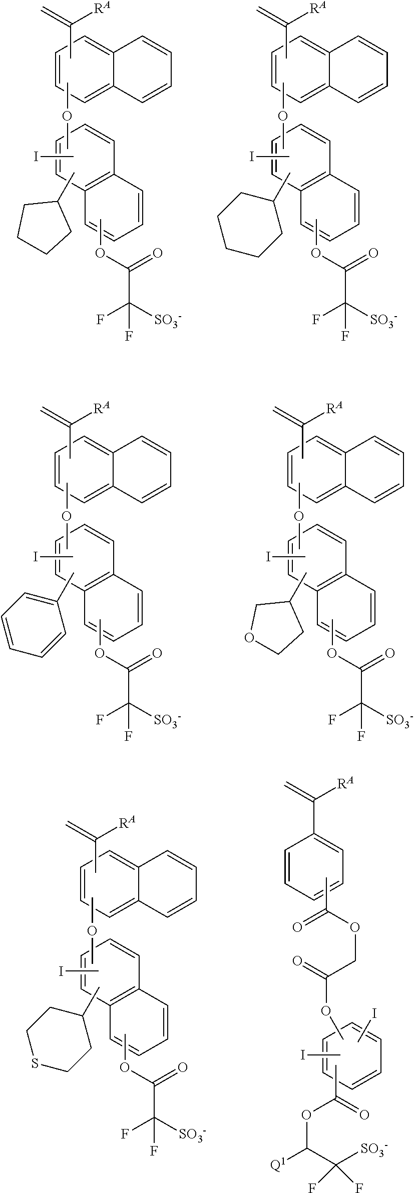

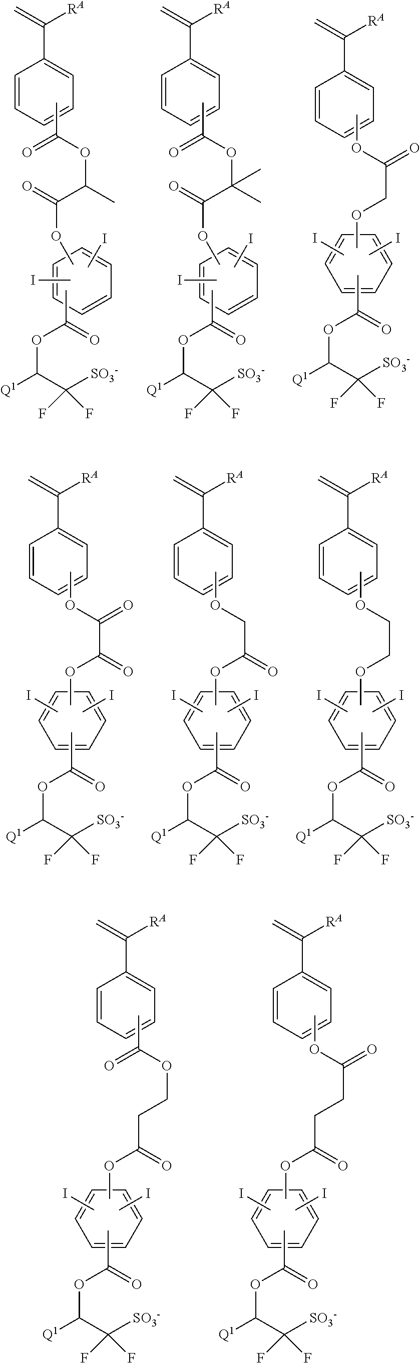

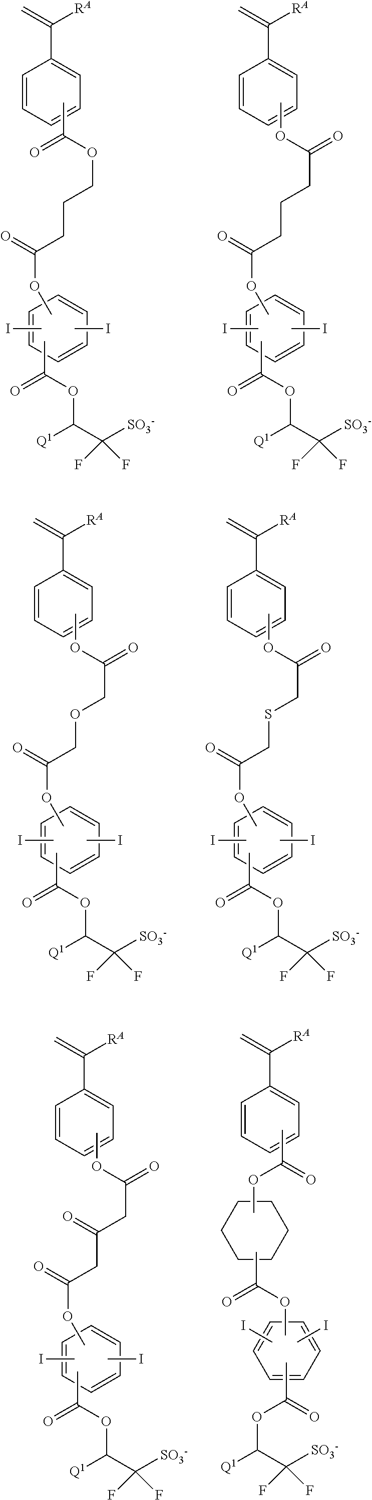

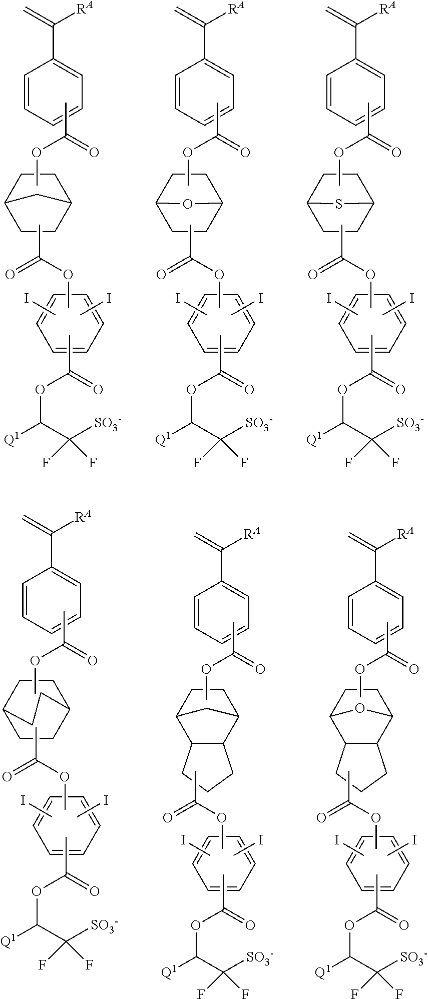

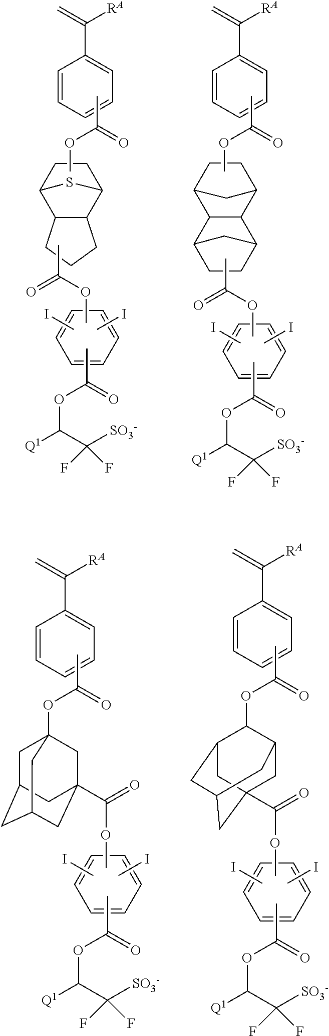

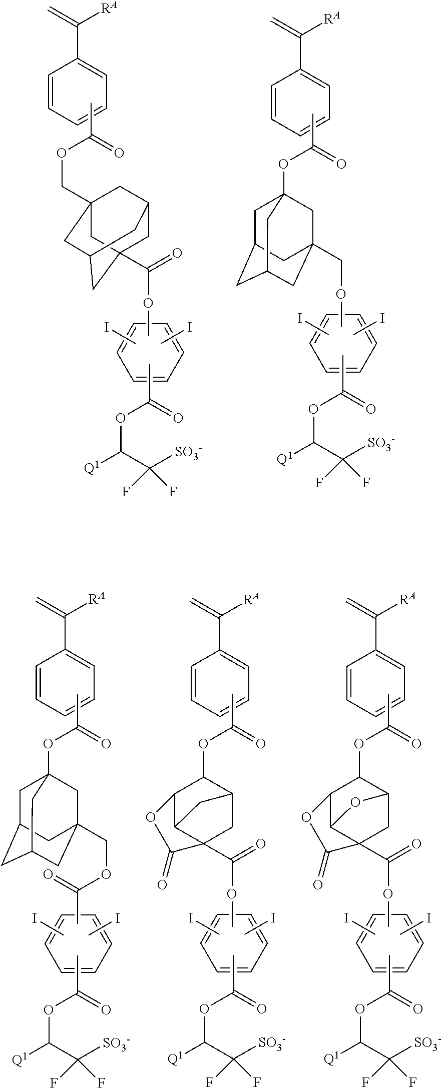

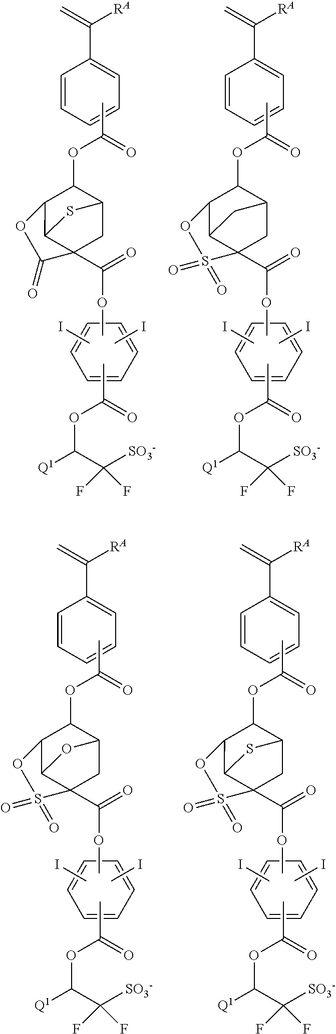

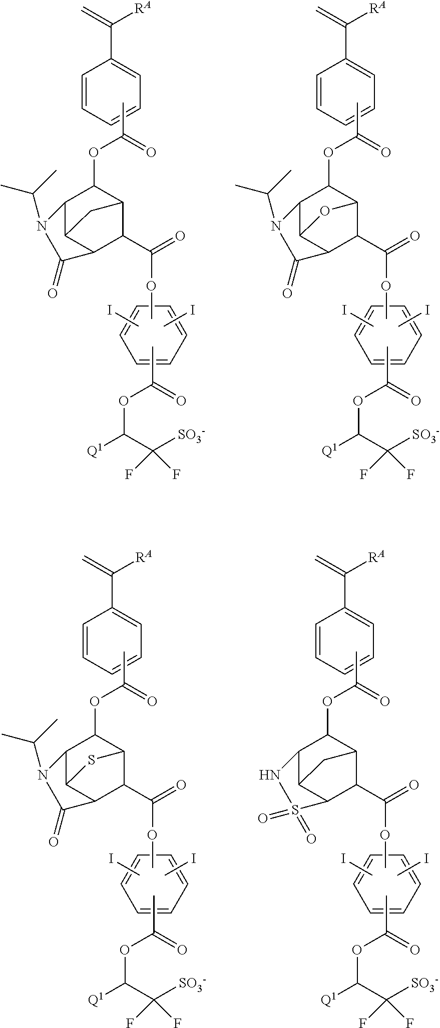

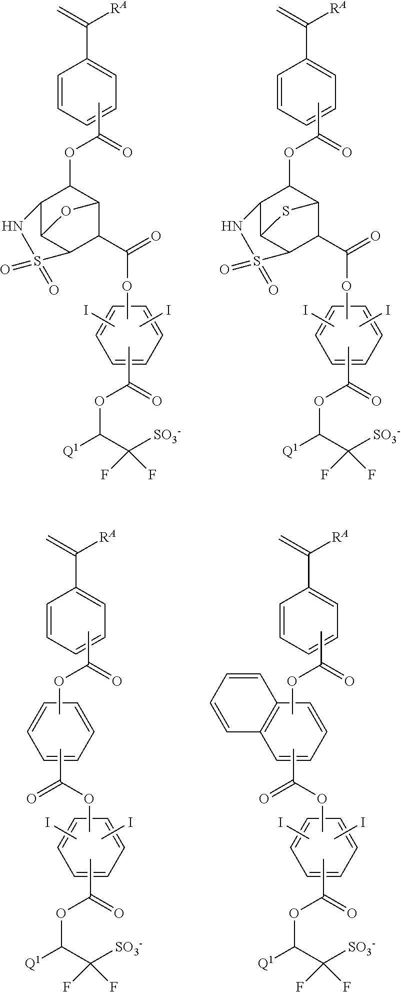

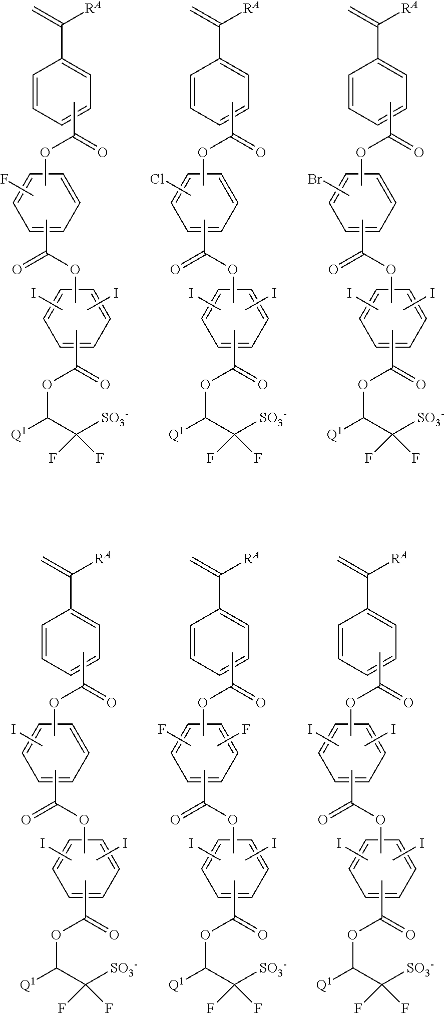

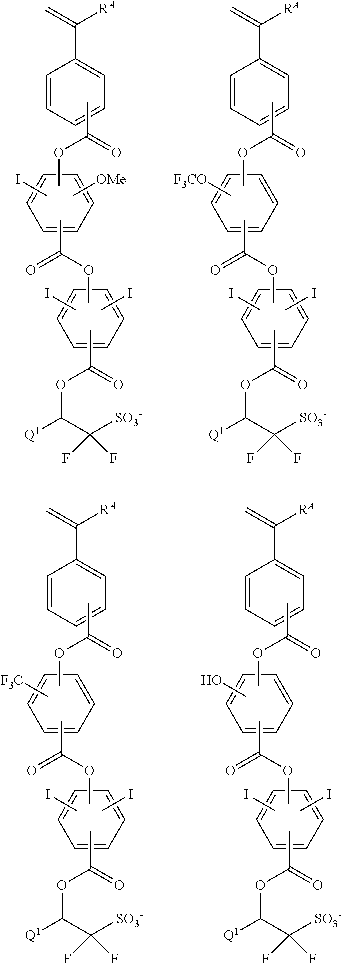

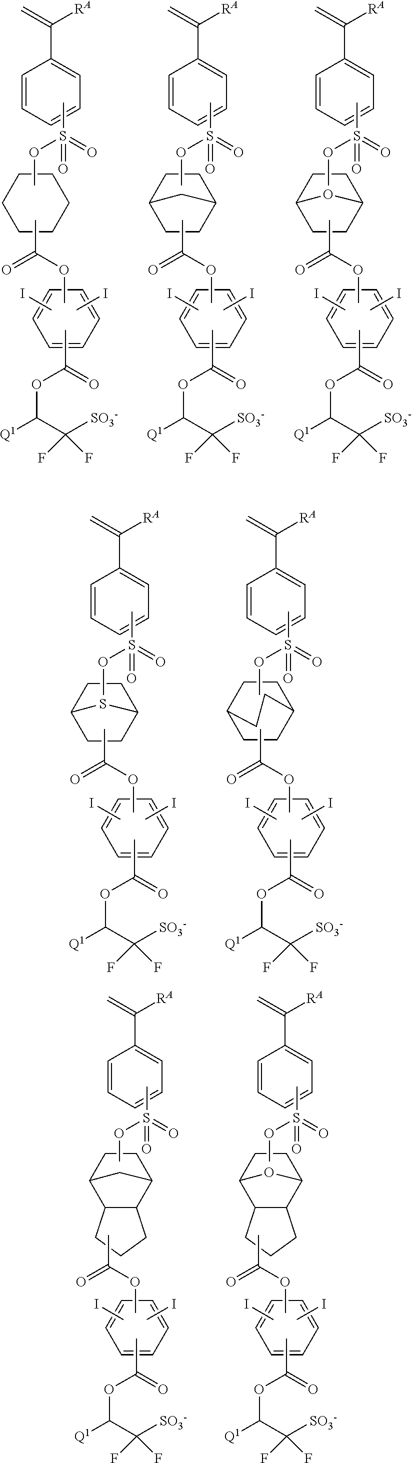

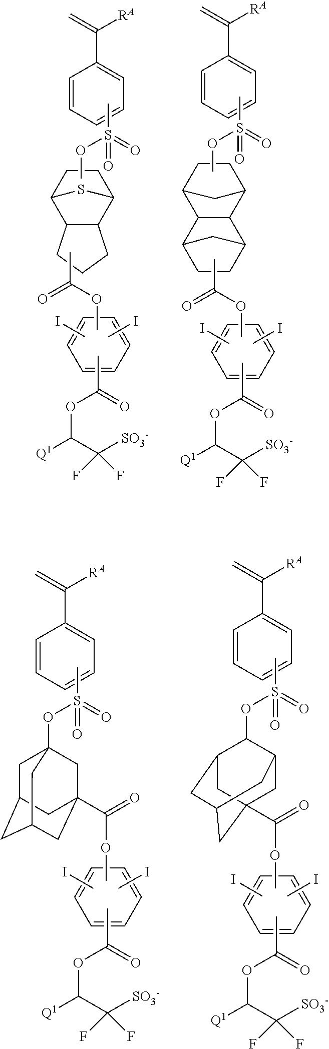

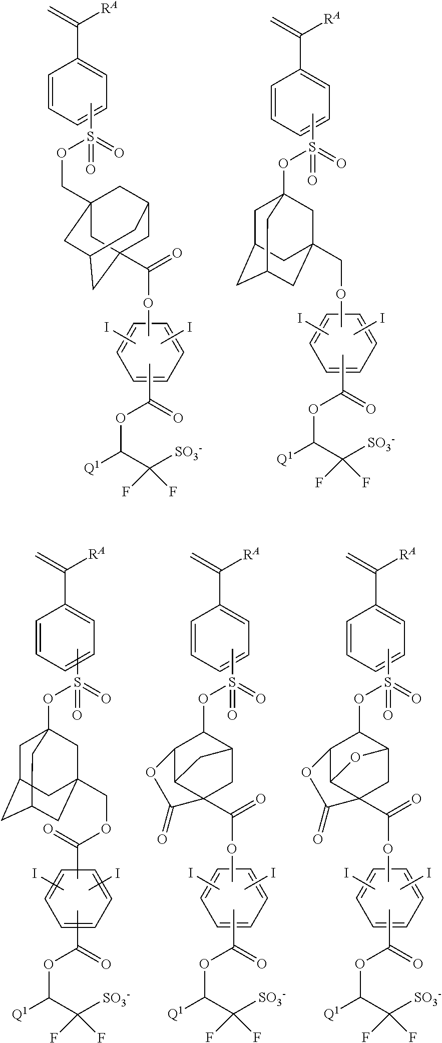

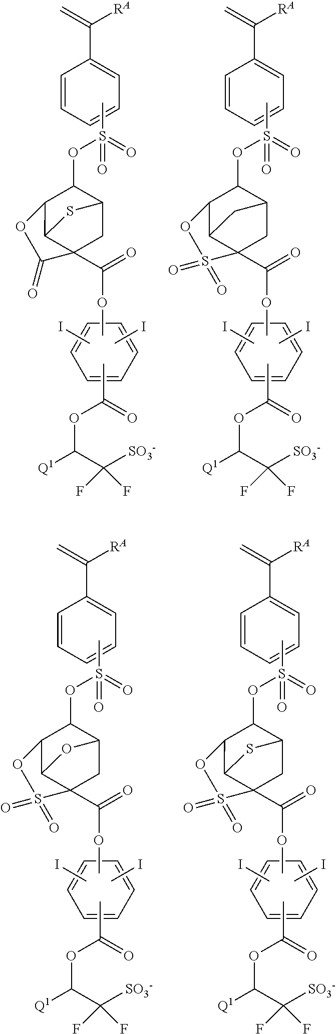

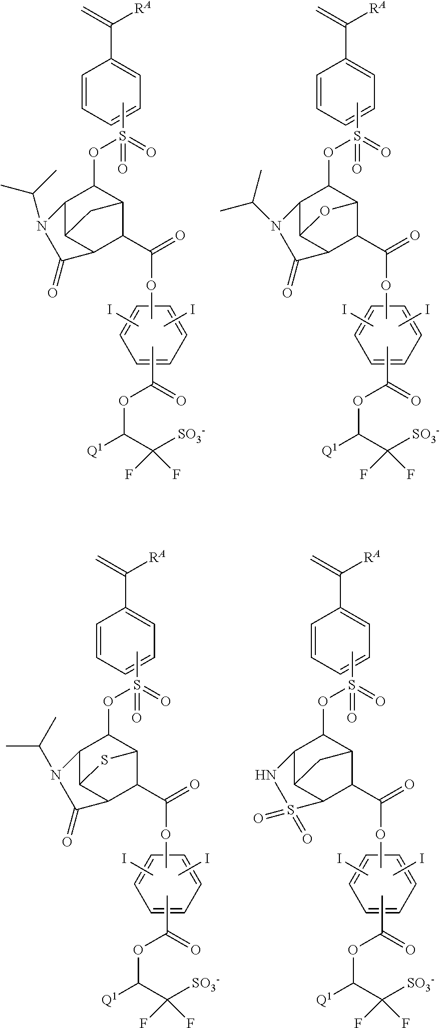

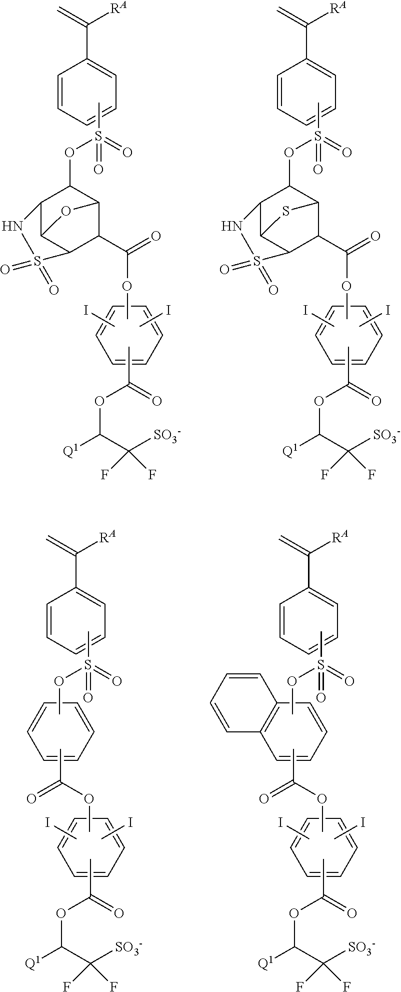

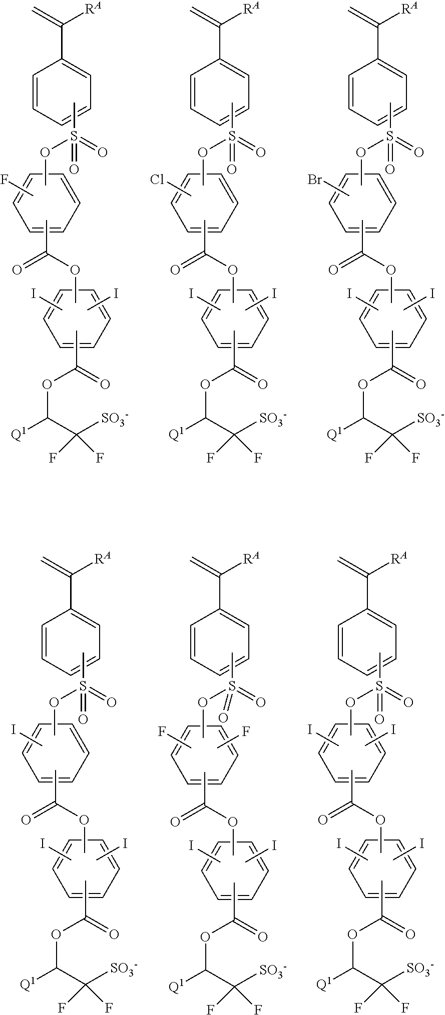

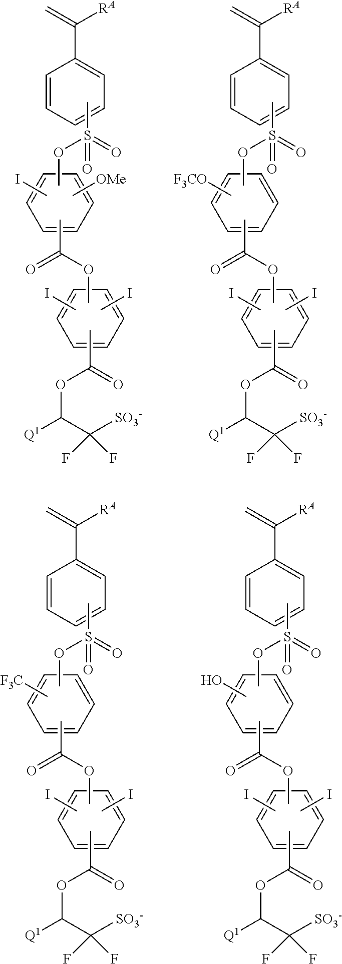

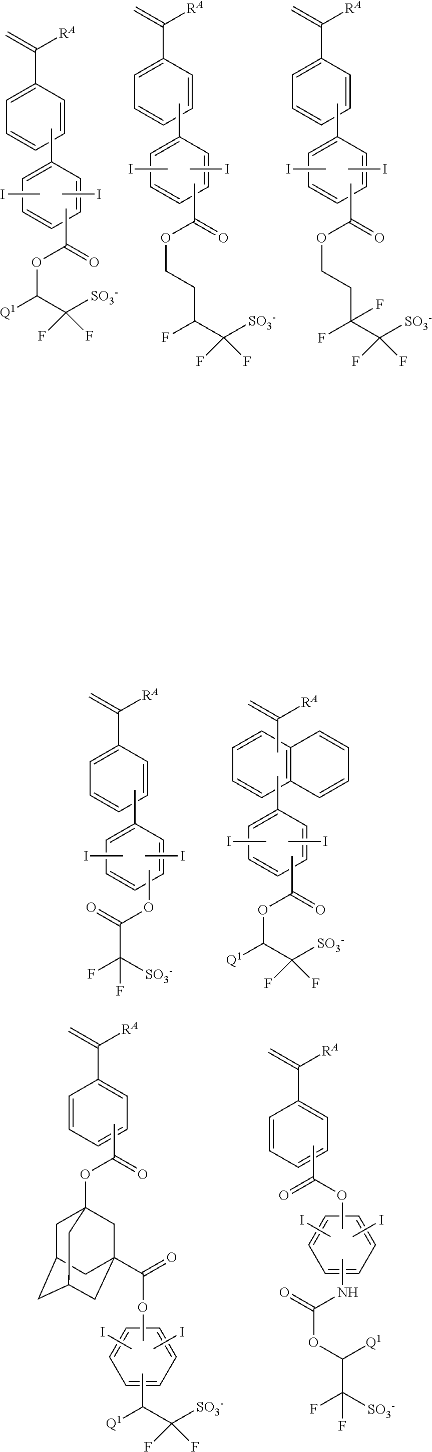

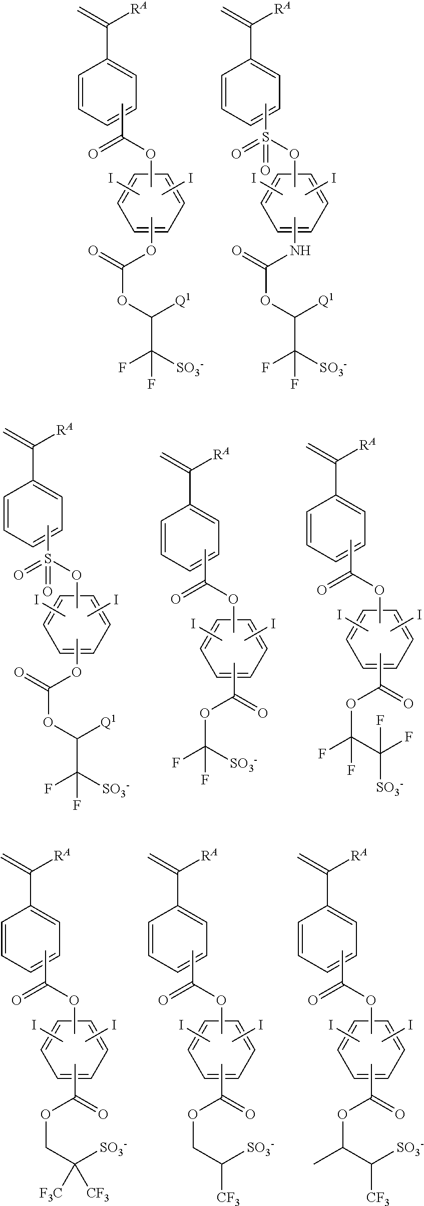

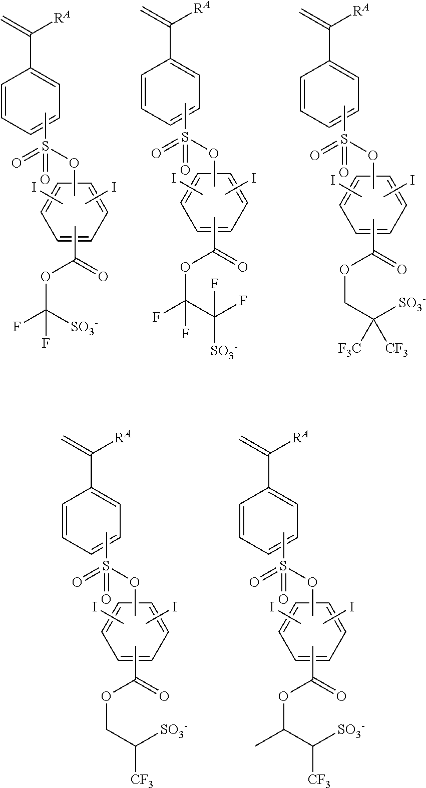

Further, as anions in repeat units (d), structures having the formula (d2) are also preferred.

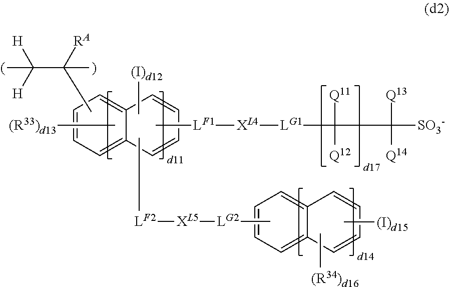

In formula (d2), d11 is 0 or 1. The sulfonium salt monomer has a benzene ring when d11 is 0, and a naphthalene ring when d11 is 1, and d11 is preferably 0 from the aspect of solvent solubility. The subscript d12 is 1, 2, 3 or 4. From the aspect of reactant availability, d12 is preferably 1, 2 or 3, more preferably 1 or 2, still more preferably 1. The subscript d13 is 0, 1 or 2. The sum of d12+d13 is from 1 ito 4 when d11 is 0, and the sum of d12+d13 is from 1 to 6 when d11 is 1.

In formula (d2), d14 is 0 or 1. The sulfonium salt monomer has a benzene ring when d14 is 0, and a naphthalene ring when d14 is 1, and d14 is preferably 0 from the aspect of solvent solubility. The subscript d15 is 1, 2, 3 or 4, preferably 1, 2 or 3. As the number of iodine atoms in the anionic structure becomes larger, the amount of absorption of EUV increases, but precipitation in the resist composition may occur due to reduced solvent solubility. Therefore, the number of iodine atoms in the anion is preferably 2, 3, 4 or 5, still more preferably 2, 3 or 4. The subscript d16 is 0, 1 or 2. The sum of d15+d16 is from 1 to 4 when d14 is 0, and the sum of d15+d16 is from 1 to 6 when d14 is 1.

In formula (d2), d17 is 0, 1, 2, 3 or 4, preferably 0, 1, 2 or 3, more preferably 1, 2 or 3, still more preferably 1.

In formula (d2), R^{A} is hydrogen, fluorine, methyl group or trifluoromethyl group. Of these, hydrogen atom and methyl group are preferred, and hydrogen is more preferred.

In formula (d2), R³³ is halogen exclusive of iodine, nitro group, a C₁-C₂₀ hydrocarbyl group which may contain a heteroatom, a C₁-C₂₀ hydrocarbyloxy group which may contain a heteroatom, a C₁-C₂₀ hydrocarbyloxy group, or a C₁-C₂₀ hydrocarbylthio group which may contain a heteroatom. The halogen exclusive of iodine is preferably fluorine, chlorine, bromine or iodine, more preferably fluorine. The hydrocarbyl group and hydrocarbyl moiety of the hydrocarbyloxy and hydrocarbylthio groups may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl and icocyl groups; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl and adamantyl groups; C₂-C₂₀ alkenyl groups such as vinyl, allyl, propenyl, butenyl and hexenyl groups; C₃-C₂₀ cyclic unsaturated hydrocarbyl groups such as a cyclohexenyl group; C₆-C₂₀ aryl groups such as phenyl and naphthyl groups; C₇-C₂₀ aralkyl groups such as benzyl, 1-phenylethyl and 2-phenylethyl groups; and combinations thereof. Some or all of hydrogen atoms of the hydrocarbyl group may be replaced by a group containing a heteroatom such as oxygen, sulfur, nitrogen or halogen, some constituent —CH₂— of the hydrocarbyl group may be replaced by a group containing a heteroatom such as oxygen, sulfur or nitrogen, and as a result, the hydrocarbyl group may contain a hydroxy group, a cyano group, fluorine, chlorine, bromine, iodine, a carbonyl group, an ether bond, an ester bond, a sulfonic ester bond, a carbonate bond, a lactone ring, a sultone ring, carboxylic anhydride (—C(═O)—O—C(═O)—), a haloalkyl group, or the like. A plurality of R³³ may be identical or different when d13 is 2.

Two R³³ may bond together to form a ring with the aromatic ring carbon atom to which they are attached when d13 is 2 or 3. Examples of the ring formed herein include cyclopropane, cyclobutane, cyclopentane, cyclohexane, norbornane and adamantane rings. Some or all of hydrogen atoms in the ring may be replaced by a group containing a heteroatom such as oxygen, sulfur, nitrogen or halogen, some constituent —CH₂— in the ring may be replaced by a group containing a heteroatom such as oxygen, sulfur or nitrogen, and as a result, the hydrocarbyl group may contain a hydroxy group, fluorine, chlorine, bromine, iodine, a cyano group, a carbonyl group, an ether bond, an ester bond, a sulfonic ester bond, a carbonate bond, a lactone ring, a sultone ring, carboxylic anhydride (—C(═O)—O—C(═O)—), a haloalkyl group, or the like.

In formula (d2), R³⁴ is halogen exclusive of iodine, nitro group, a C₁-C₂₀ hydrocarbyl group which may contain a heteroatom, a C₁-C₂₀ hydrocarbyloxy group which may contain a heteroatom, a C₁-C₂₀ hydrocarbyloxy group, or a C₁-C₂₀ hydrocarbylthio group which may contain a heteroatom. Examples of the halogen exclusive of iodine include fluorine, chlorine, bromine, and iodine. The hydrocarbyl group and hydrocarbyl moiety of the hydrocarbyloxy and hydrocarbylthio groups may be saturated or unsaturated and straight, branched or cyclic. Examples of the hydrocarbyl group are as exemplified above as a hydrocarbyl group R³³ but not limited thereto. A plurality of R³⁴ may be identical or different when d16 is 2.

Two R³⁴ may bond together to form a ring with the carbon atoms to which they are attached when d16 is 2. Of the rings, 5 to 8-membered rings are preferred.

In formula (d2), L^{F1} L^{F2}, L^{G1} and LG² are each independently a single bond, ether bond, ester bond, sulfonic ester bond, amide bond, sulfonic amide bond, carbonate bond or carbamate bond, Inter alia, L^{F1} is preferably a single bond, ether bond, ester bond or sulfonic ester bond, more preferably ester bond or sulfonic ester bond. L^{F2} is preferably a single bond, ether bond, ester bond or sulfonic ester bond, more preferably an ester bond or sulfonic ester bond. L^{G1} is preferably a single bond, ether bond, ester bond or sulfonic ester bond, more preferably a single bond, ether bond or ester bond. LG² is preferably a single bond, ether bond, ester bond or sulfonic ester bond, more preferably a single bond, ether bond or ester bond.

Preferably, L^{F1} and L^{F2} are preferably attached to adjacent carbon atoms on the aromatic ring. Here, the substituent containing a fluorosulfonate anion structure and the substituent containing an aromatic ring substituted with iodine are positioned spatially closer to each other, and thus, the sensitivity is expected to be higher.

In formula (d2), X^{L4} and X^{L5} are each independently a single bond, or a C₁-C₄₀ hydrocarbylene group which may contain a heteroatom. The hydrocarbylene group may be straight, branched or cyclic, and examples thereof include alkanediyl, cyclic saturated hydrocarbylene, and arylene groups. Examples of the heteroatom include oxygen, nitrogen and sulfur atoms. Examples of the C₁-C₄₀ hydrocarbylene group XM and X^{L} which may contain a heteroatom include X^{L}-0 to X^{L}-58 exemplified for formula (Z) as examples of the C₁-C₄₀ hydrocarbylene groups X^{L1} and X^{L2} which may contain a heteroatom. Of these, X^{L}-0 to X^{L}-22, X^{L}-29 to X^{L}-34, and X^{L}-47 to X^{L}-58 are preferred.

In formula (d2), Q¹¹ and Q¹² are each independently hydrogen, fluorine or a C₁-C₆ fluorinated saturated hydrocarbyl group. The C₁-C₆ fluorinated saturated hydrocarbyl group is preferably a trifluoromethyl group.

In formula (d2), Q¹³ and Q¹⁴ are each independently fluorine or a C₁-C₆ fluorinated saturated hydrocarbyl group. The C₁-C₆ fluorinated saturated hydrocarbyl group is preferably a trifluoromethyl group. More preferably, Q¹³ and Q¹⁴ are fluorine.

Preferred examples of the partial structure —[C(Q¹¹)(Q¹²)]_{d17}-C(Q¹³)(Q¹⁴)-SO₃⁻ in formula (d2) are shown below, but not limited thereto. In the following formulae, * designates a point of attachment to L^{G1}

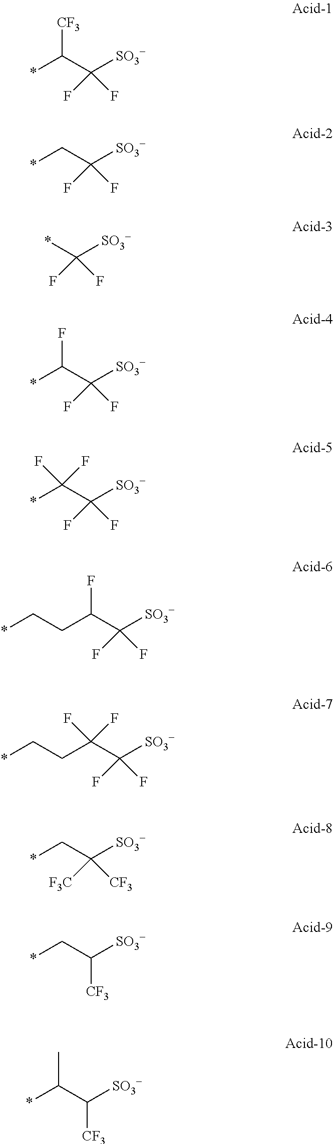

Of these, Acid-1 to Acid-7 are preferred, and Acid-1 to Acid-3, Acid-6 and Acid-7 are more preferred.

Of the anions of formula (d2), a structure having the formula (d2-1) is preferred.

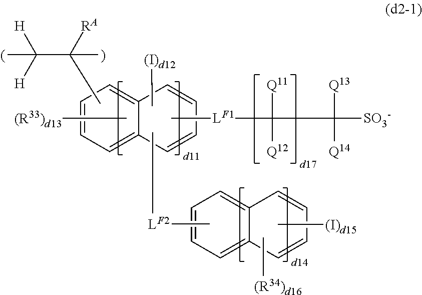

Herein, R^{A}, R³³, R³⁴, L^{F1}, L^{F2}, Q¹¹ to Q¹⁴ and d11 to d17 are as defined above.

Of the anions of formula (d2-1), a structure having the formula (d2-2) is preferred.

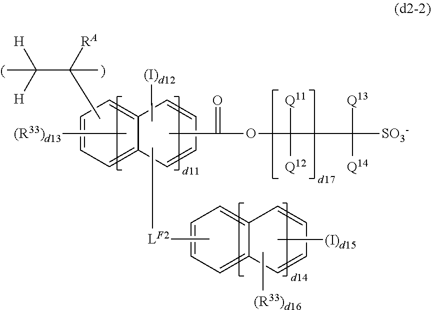

Herein, R^{A}, R³³, R³⁴, L^{F2}, Q¹¹, Q¹² and d11 to d17 are as defined above.

Examples of the anion having formula (d2) are shown below, but not limited thereto. In the following formulae, R^{A} and Q¹¹ are as defined above, and Me is a methyl group. The bonding positions of the substituents on the aromatic ring may be interchanged.

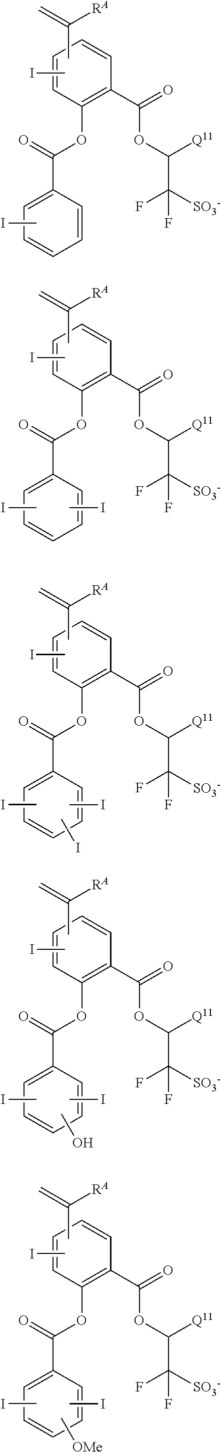

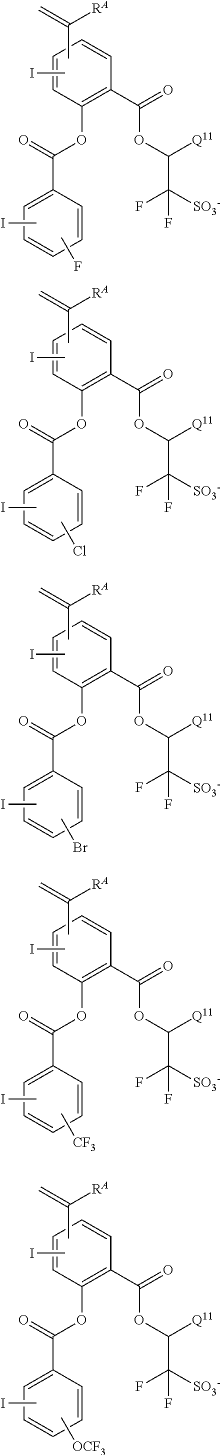

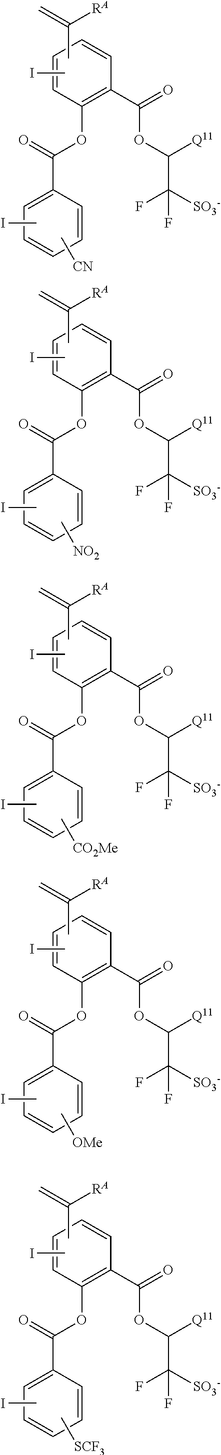

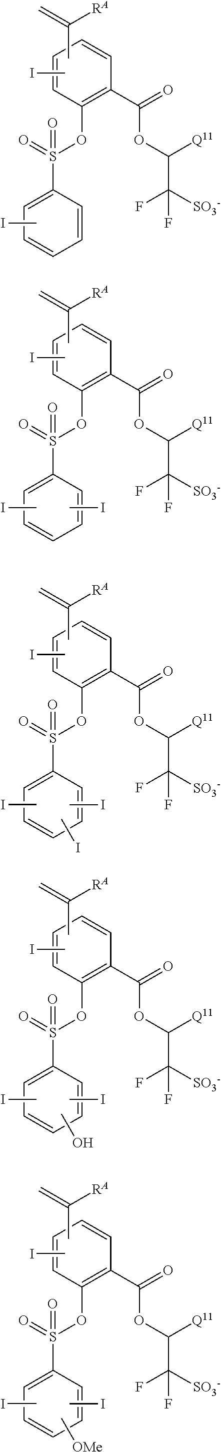

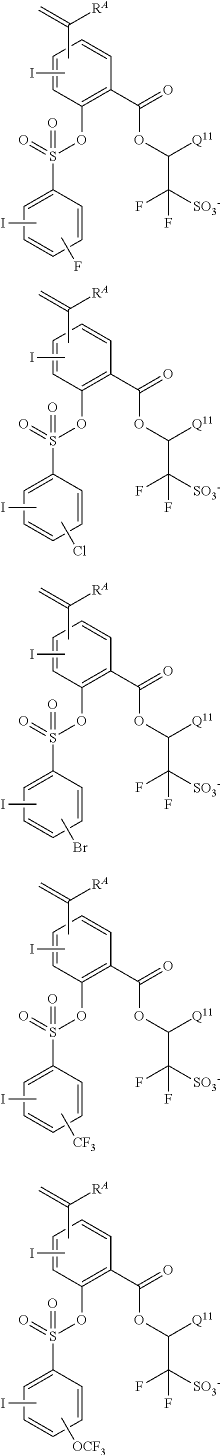

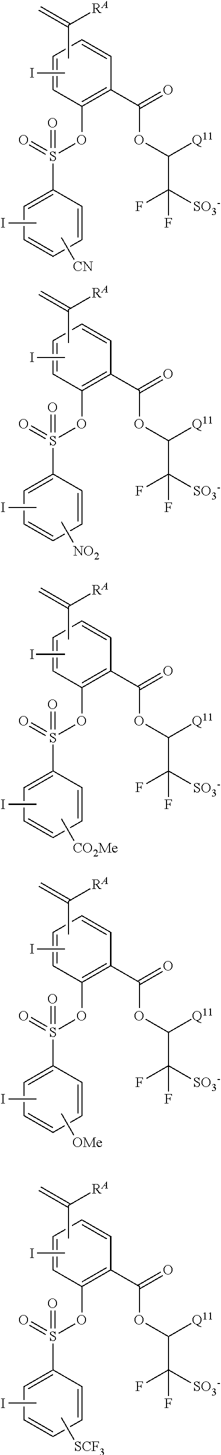

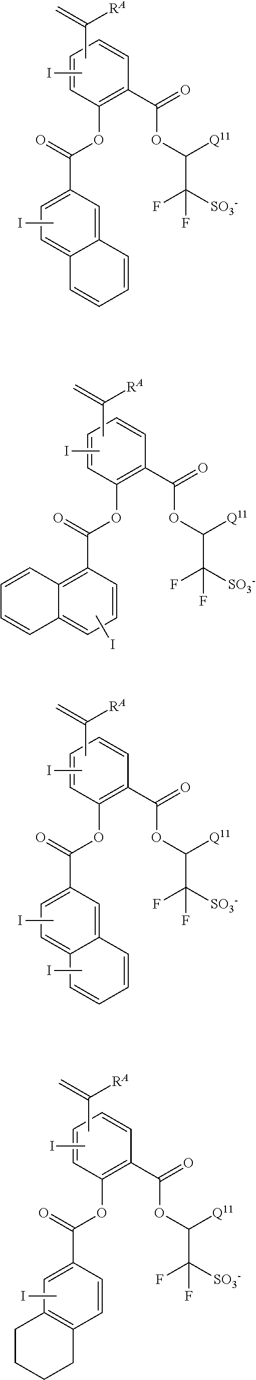

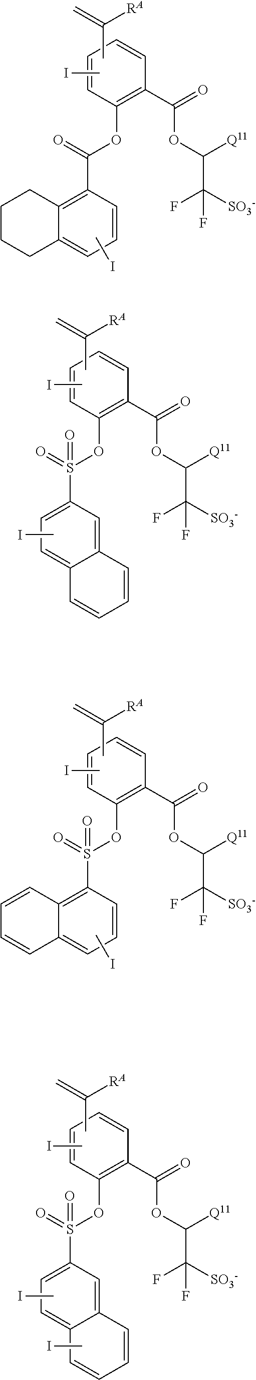

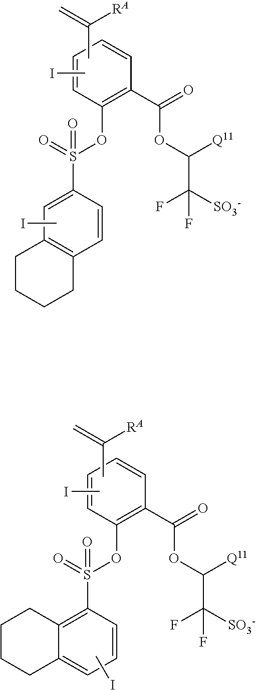

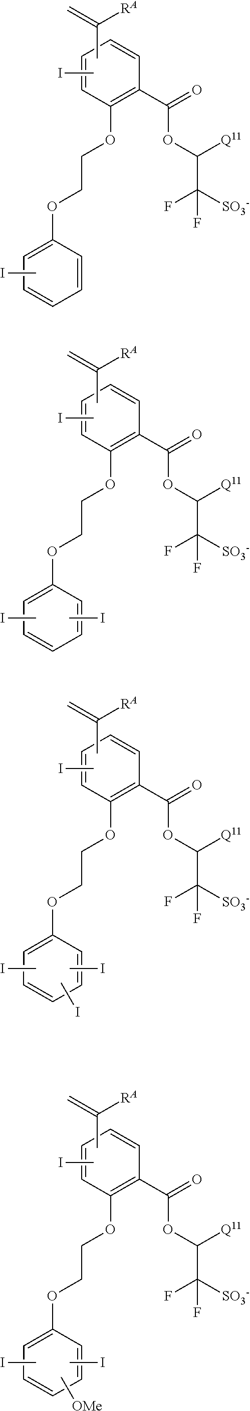

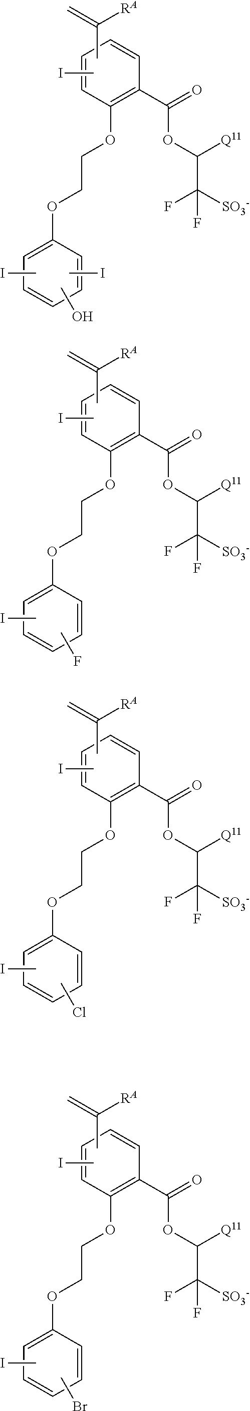

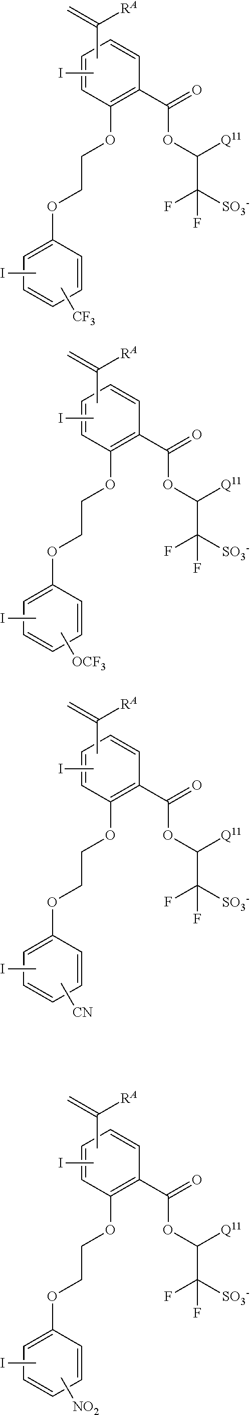

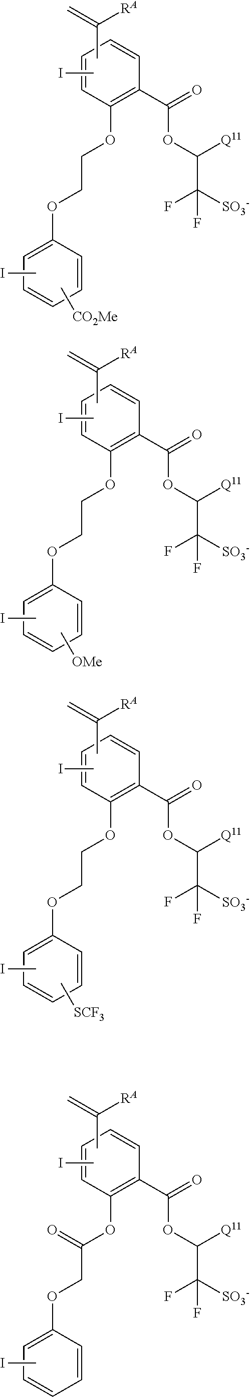

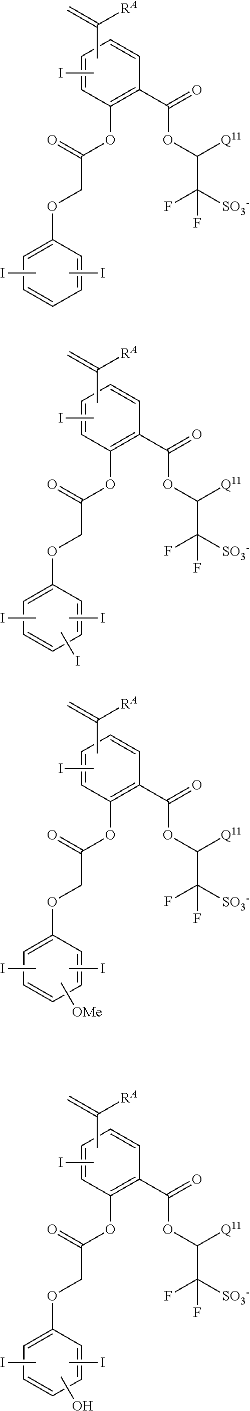

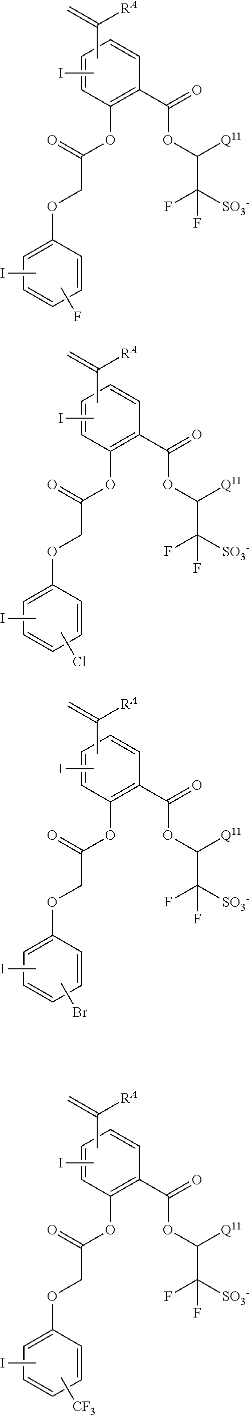

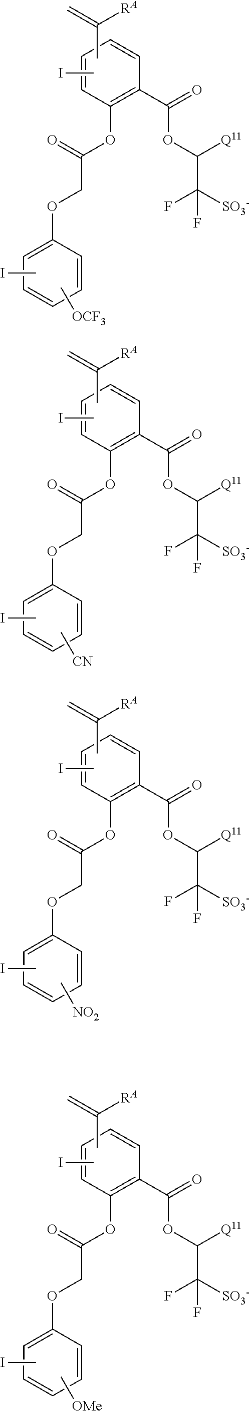

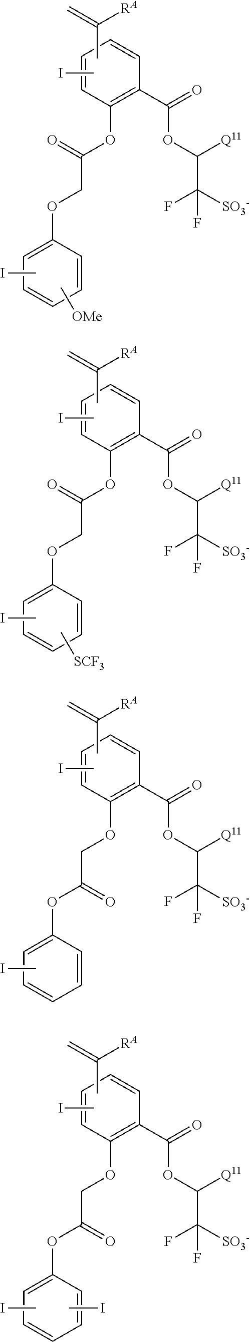

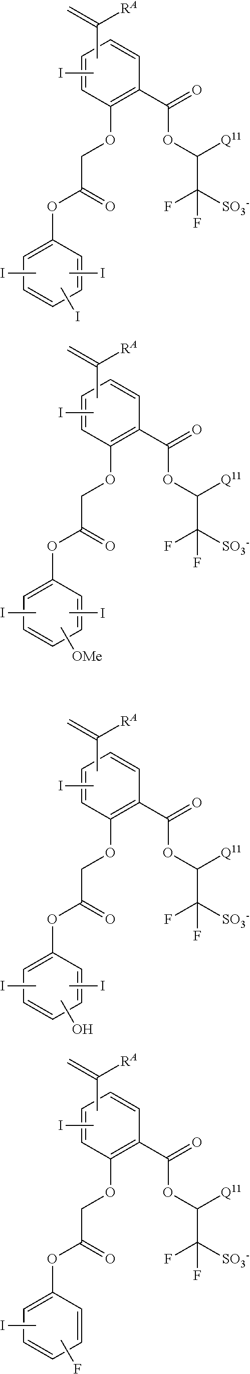

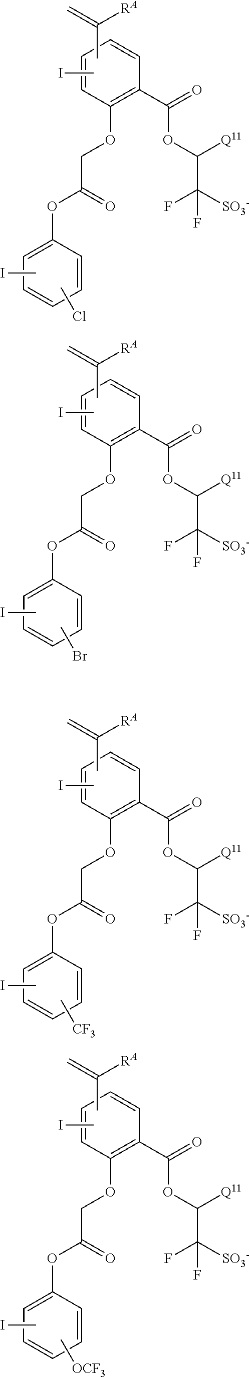

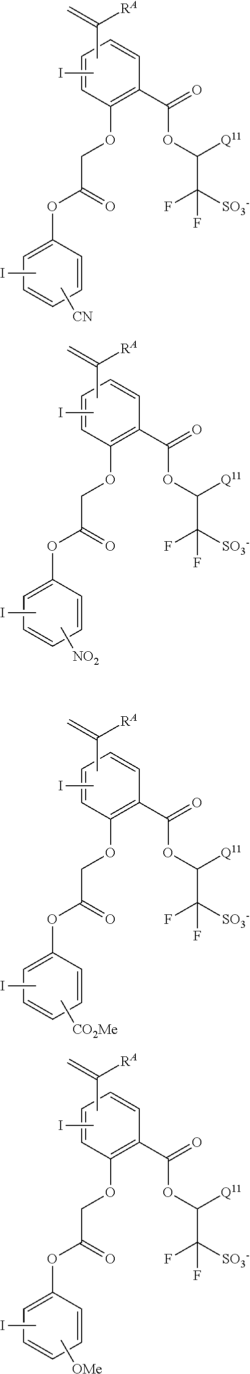

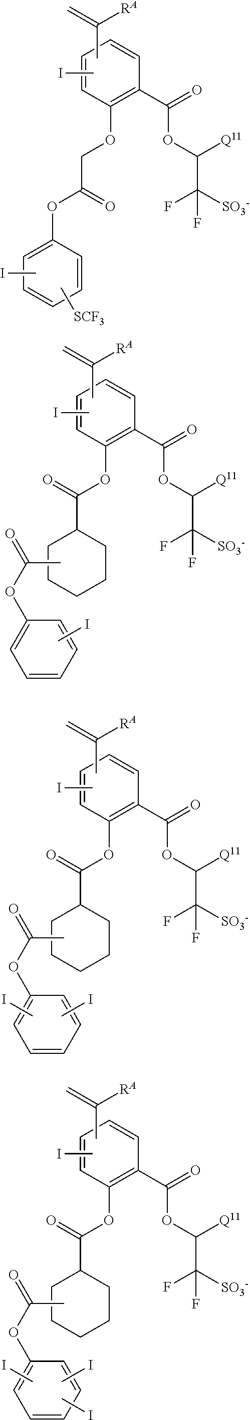

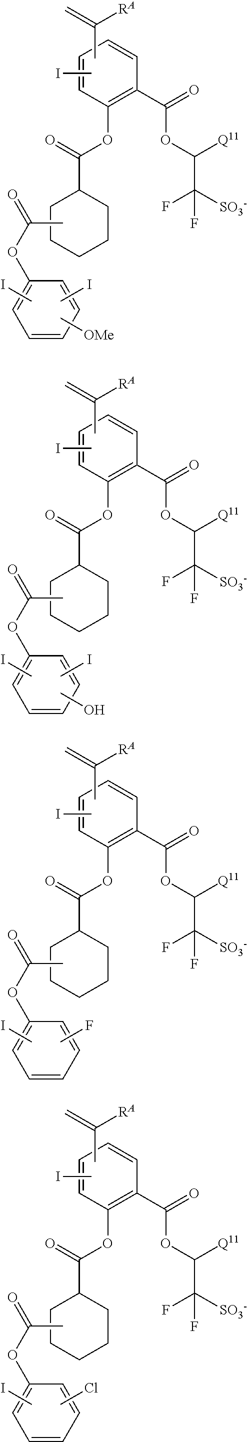

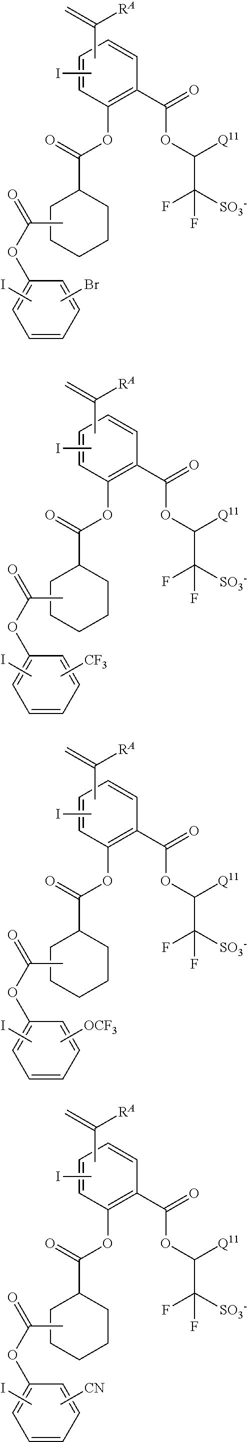

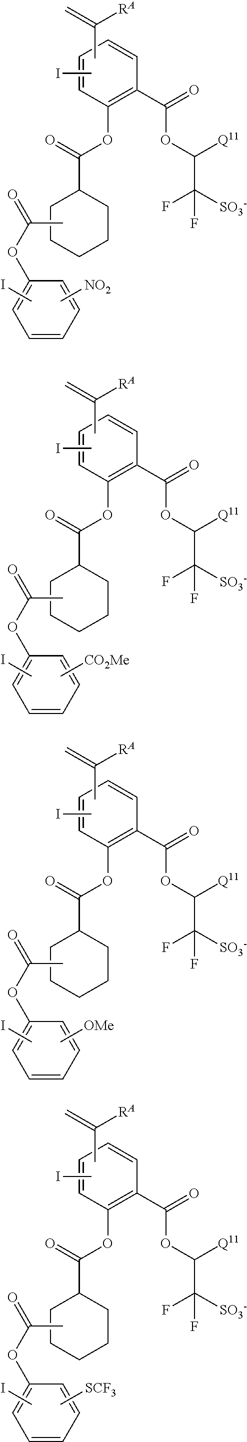

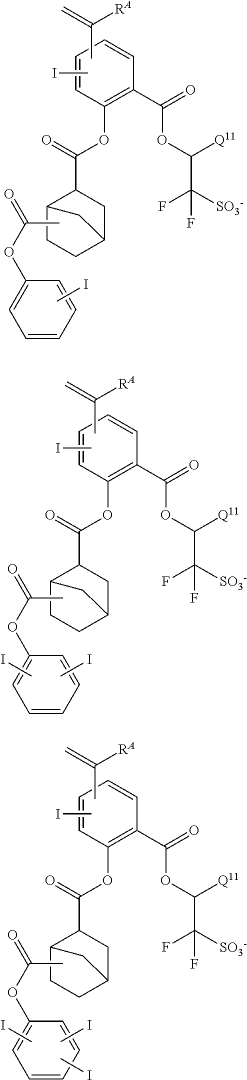

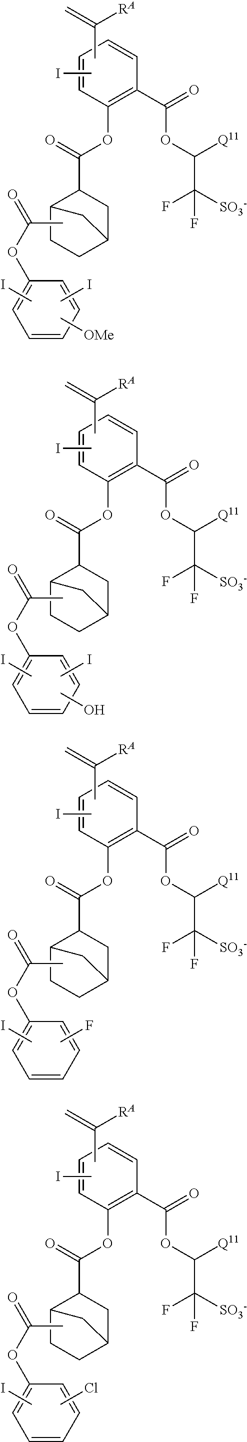

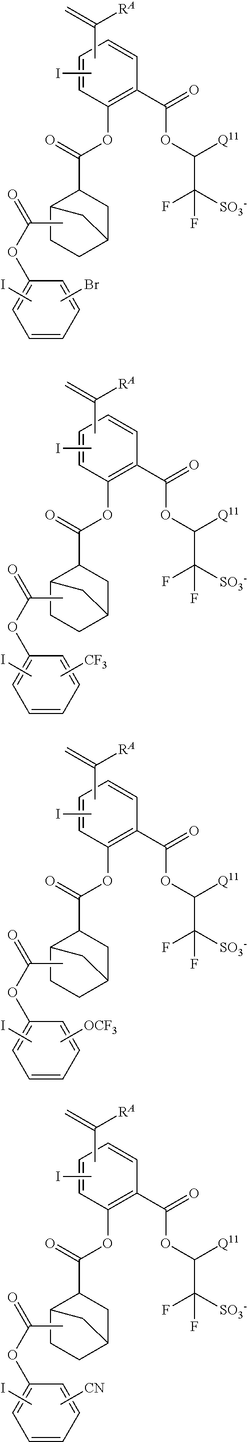

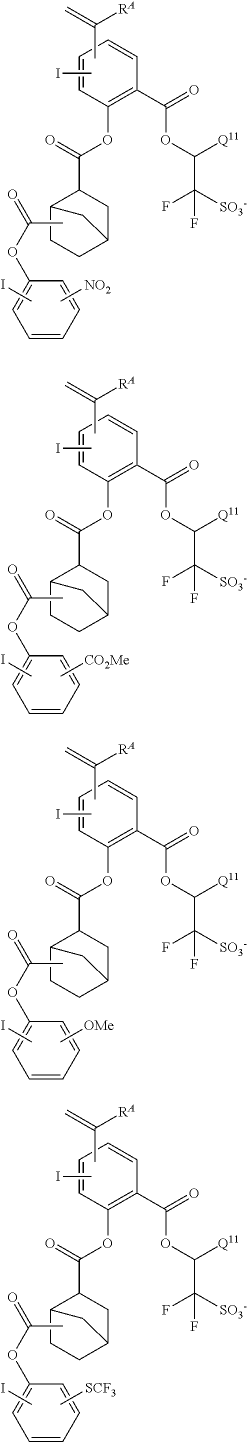

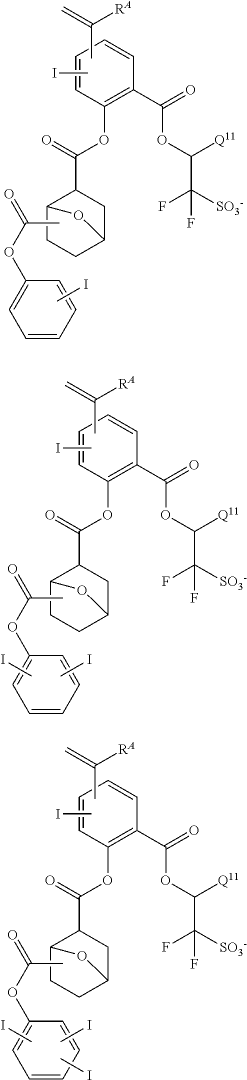

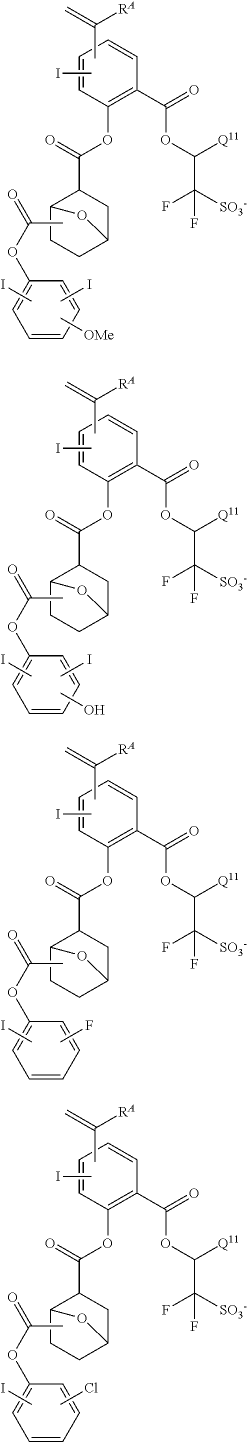

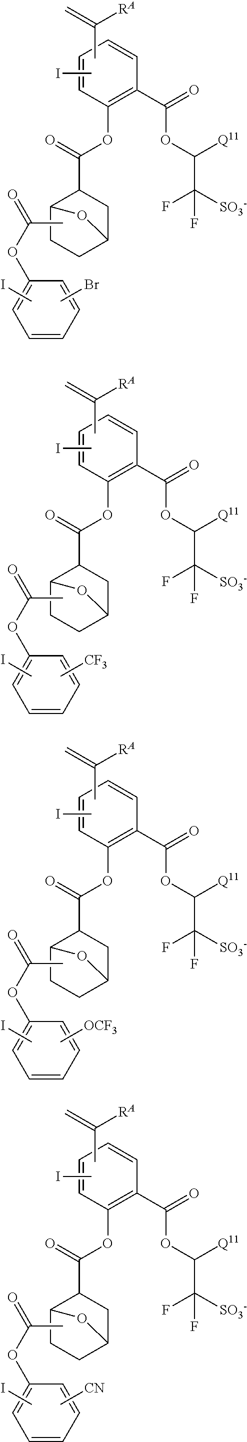

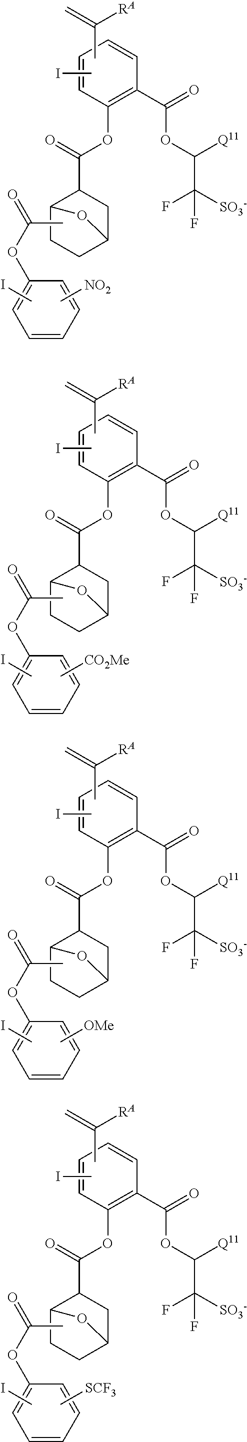

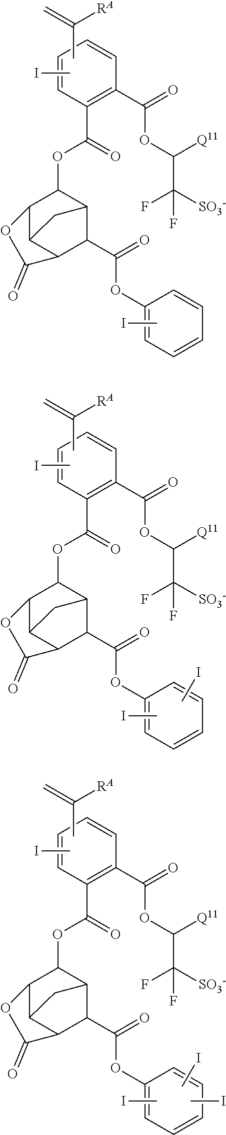

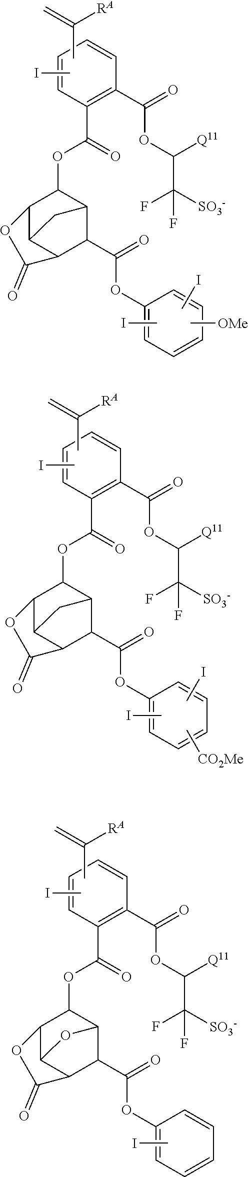

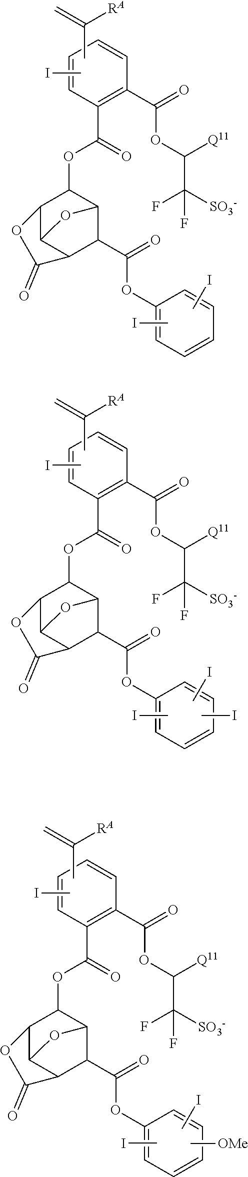

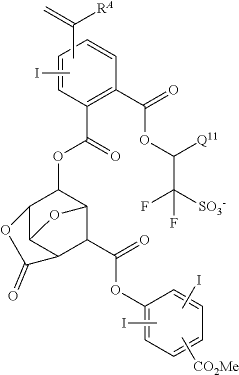

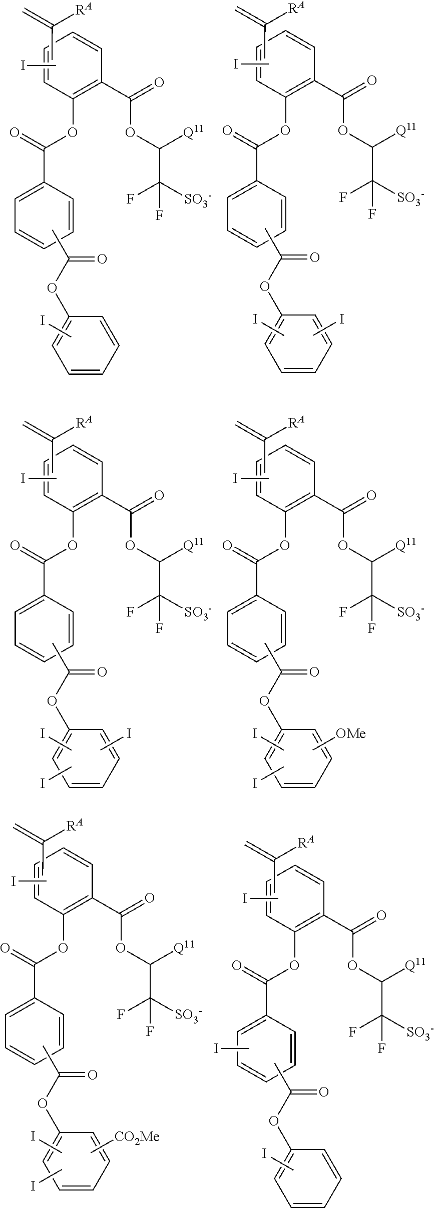

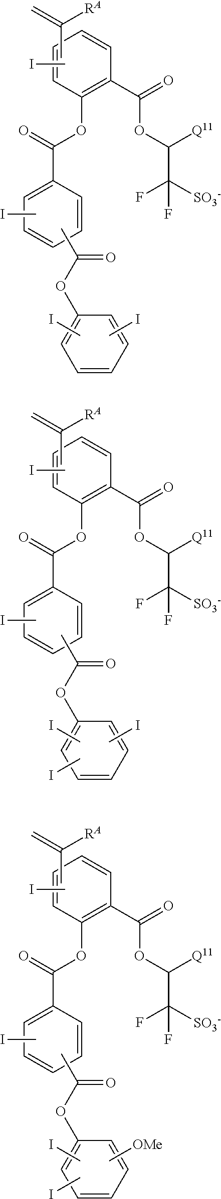

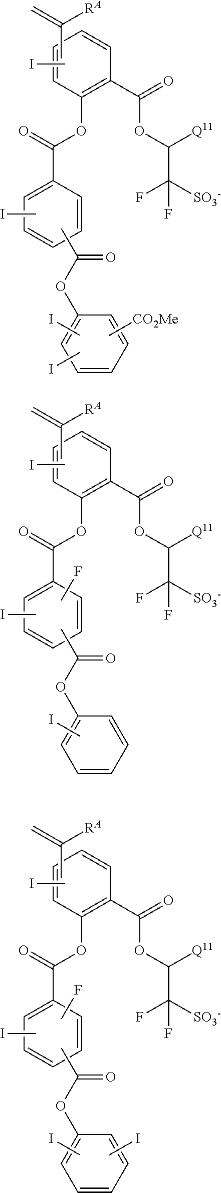

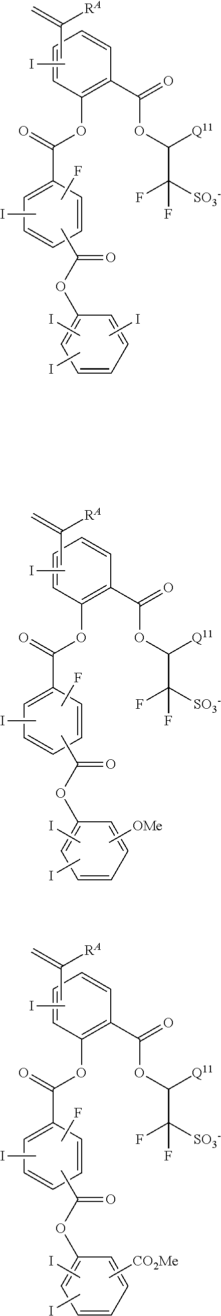

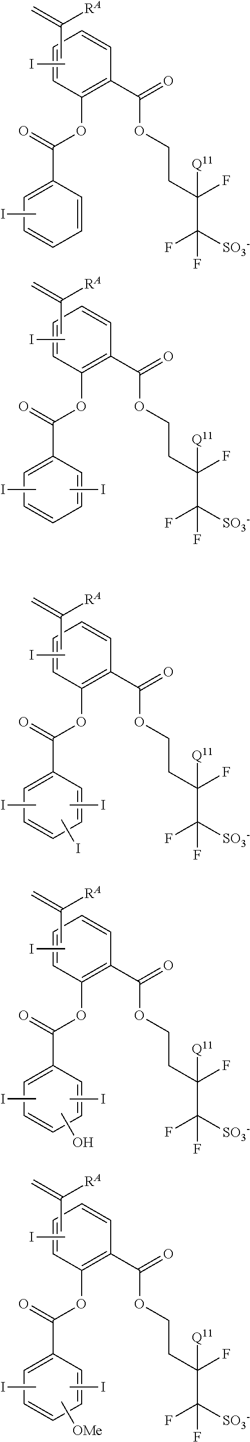

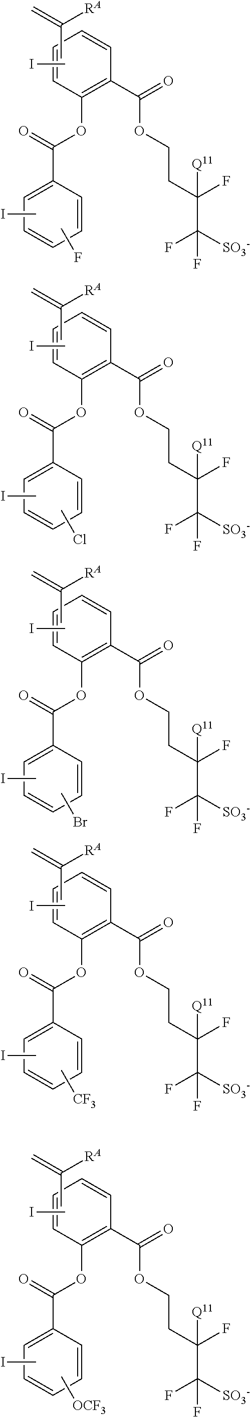

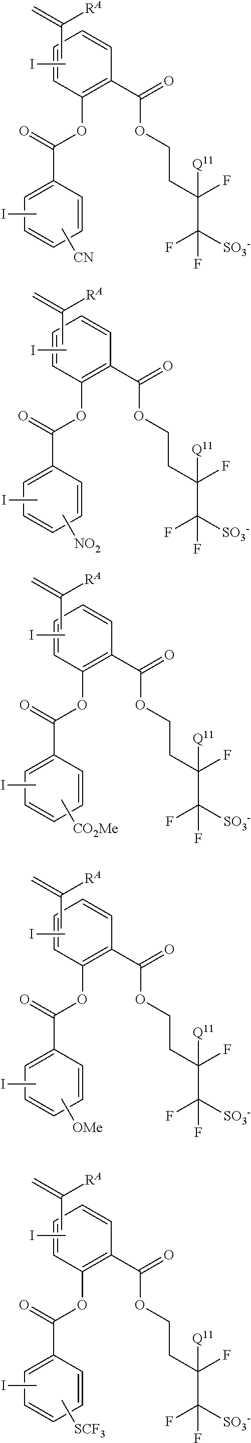

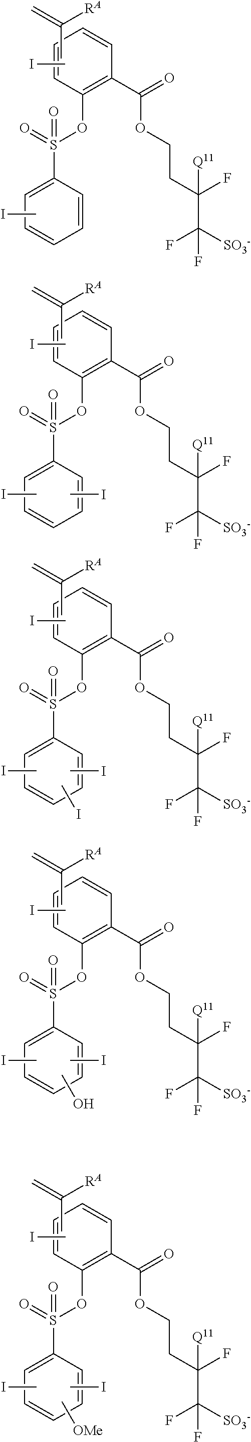

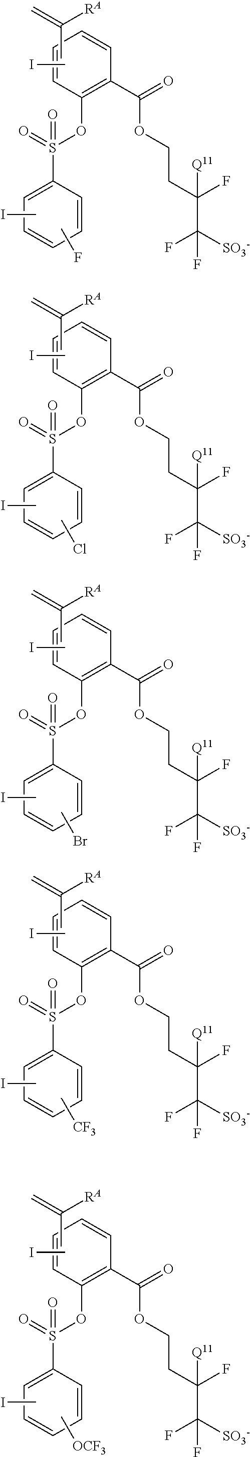

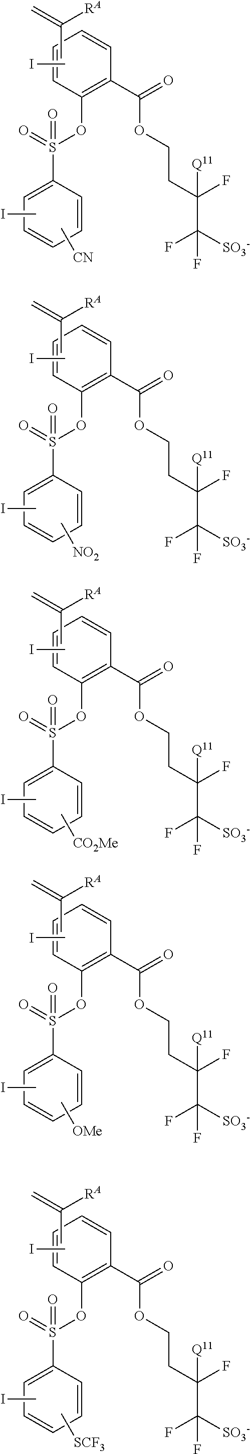

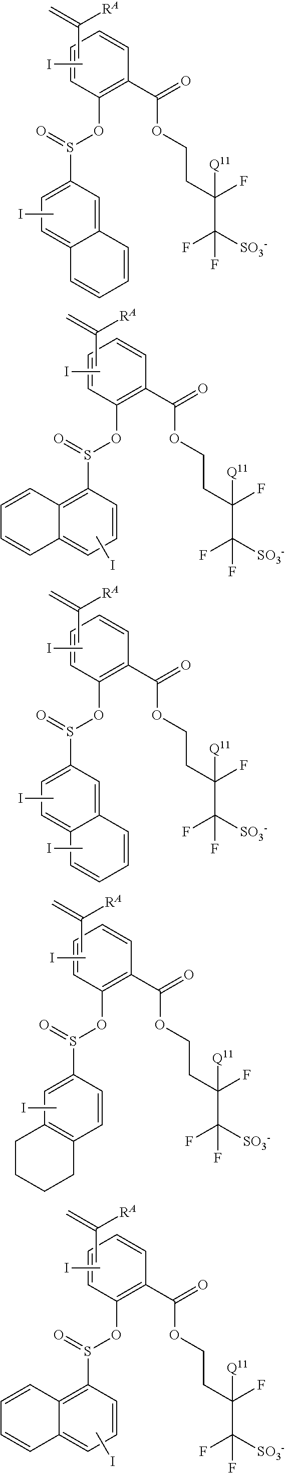

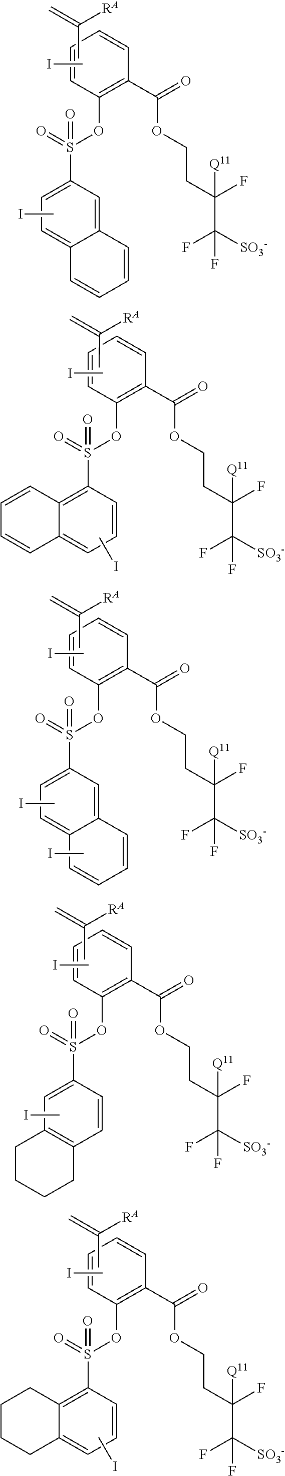

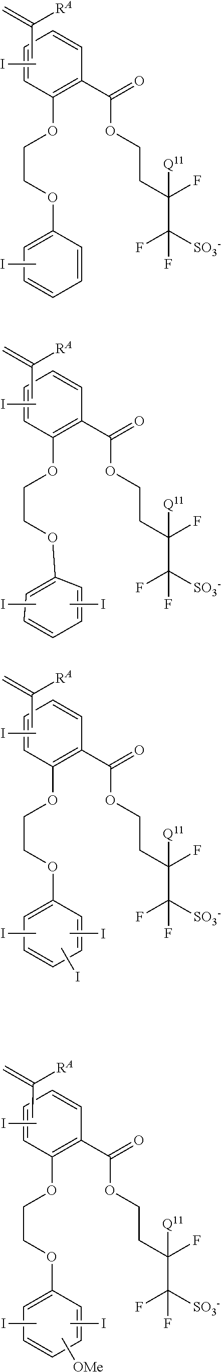

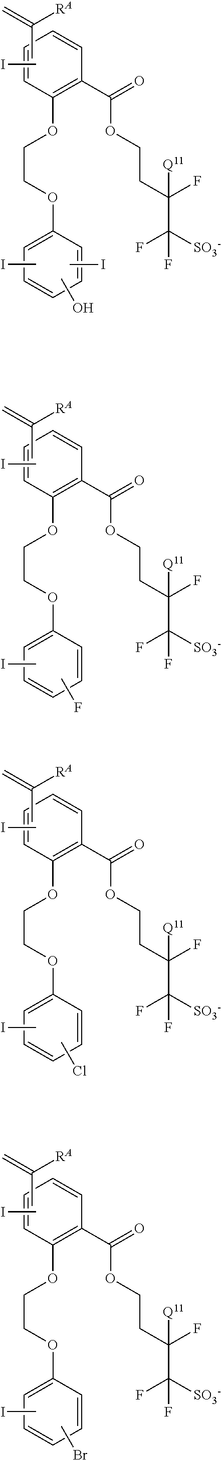

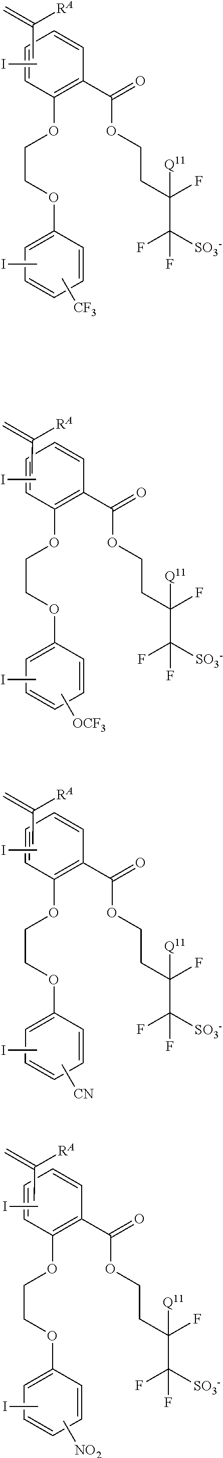

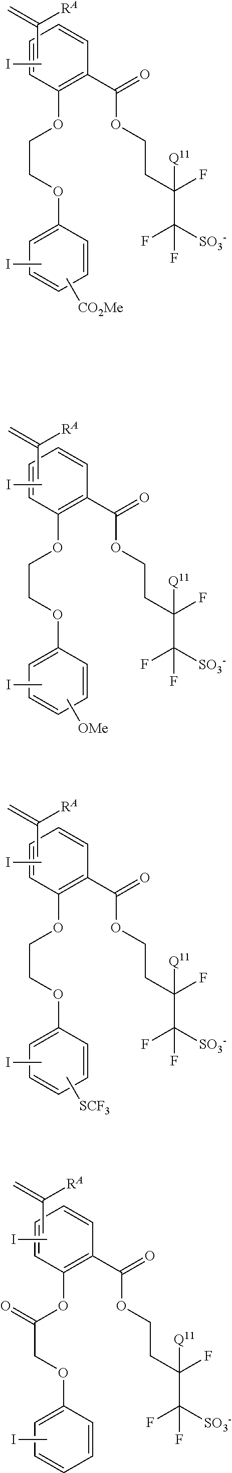

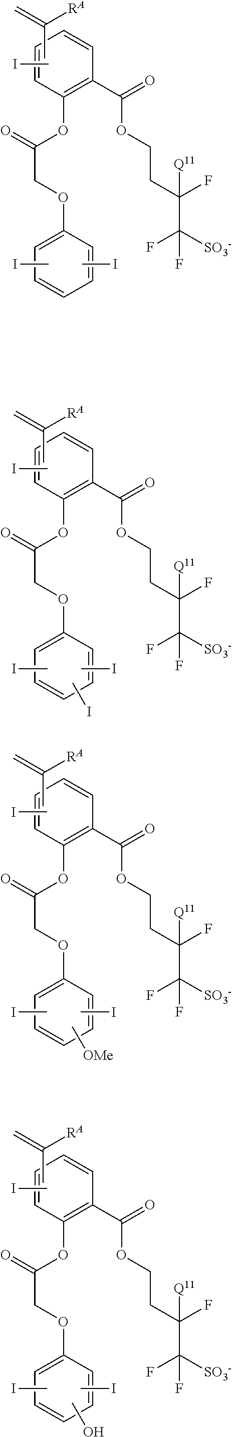

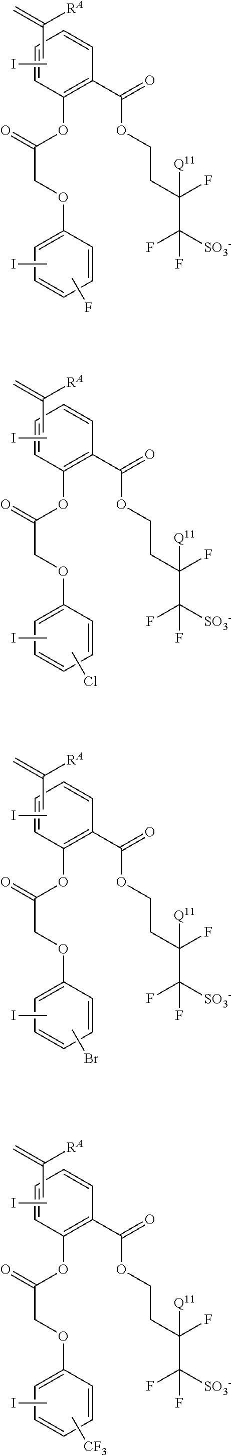

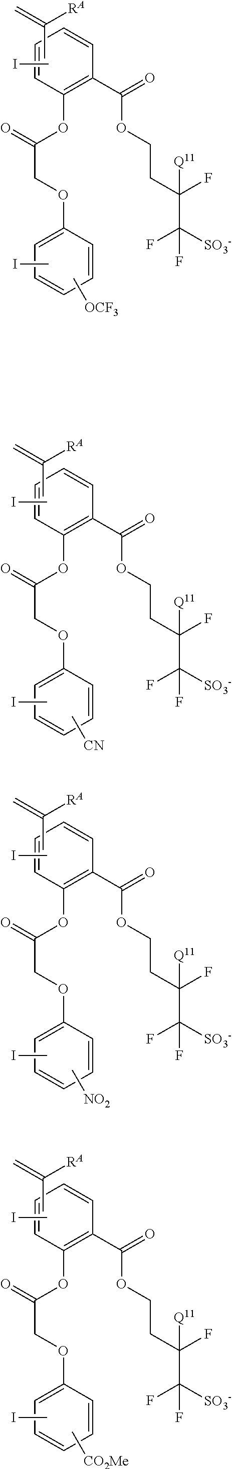

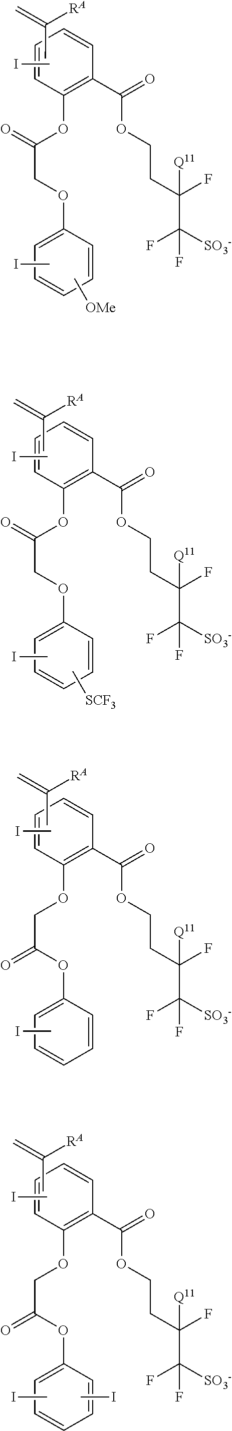

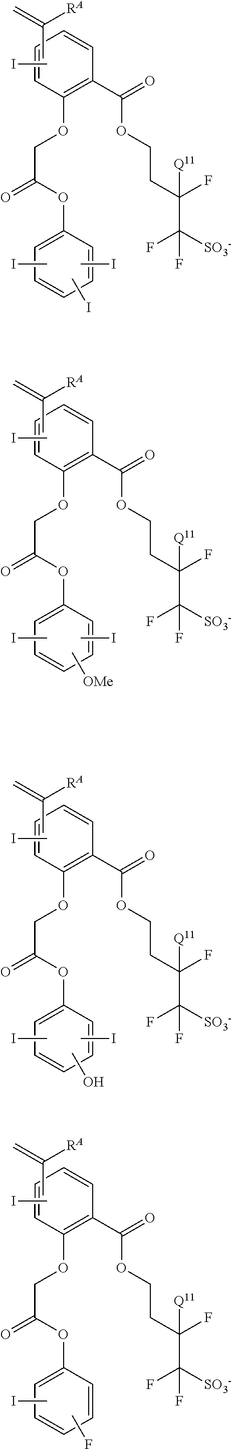

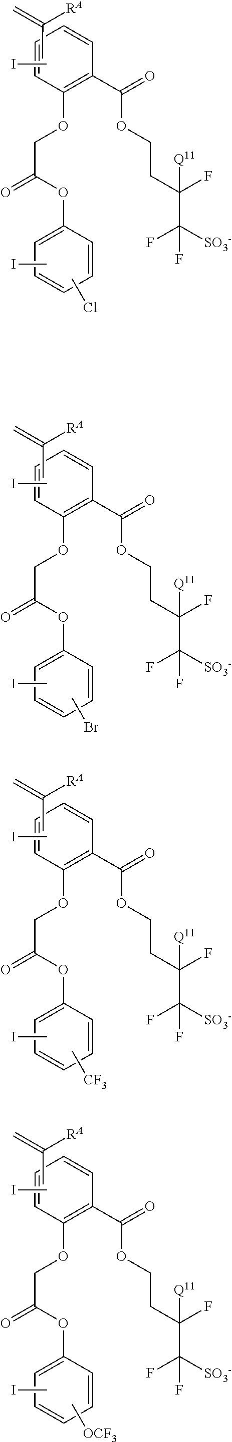

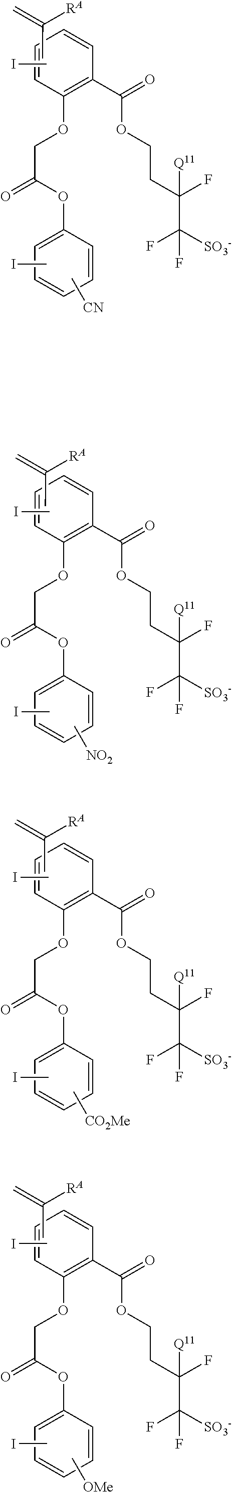

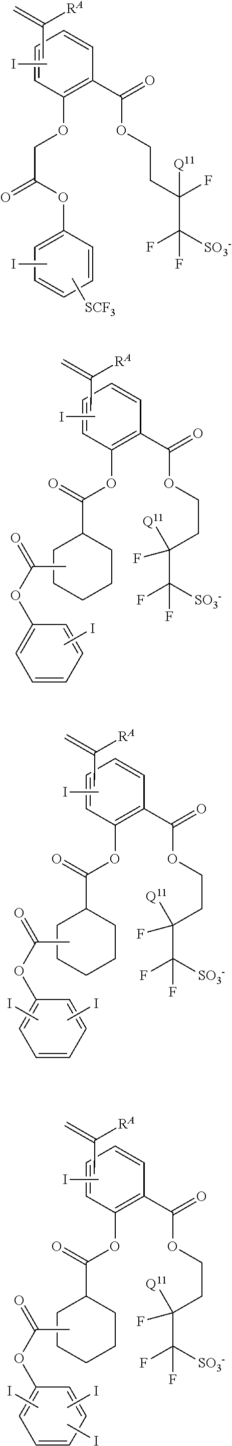

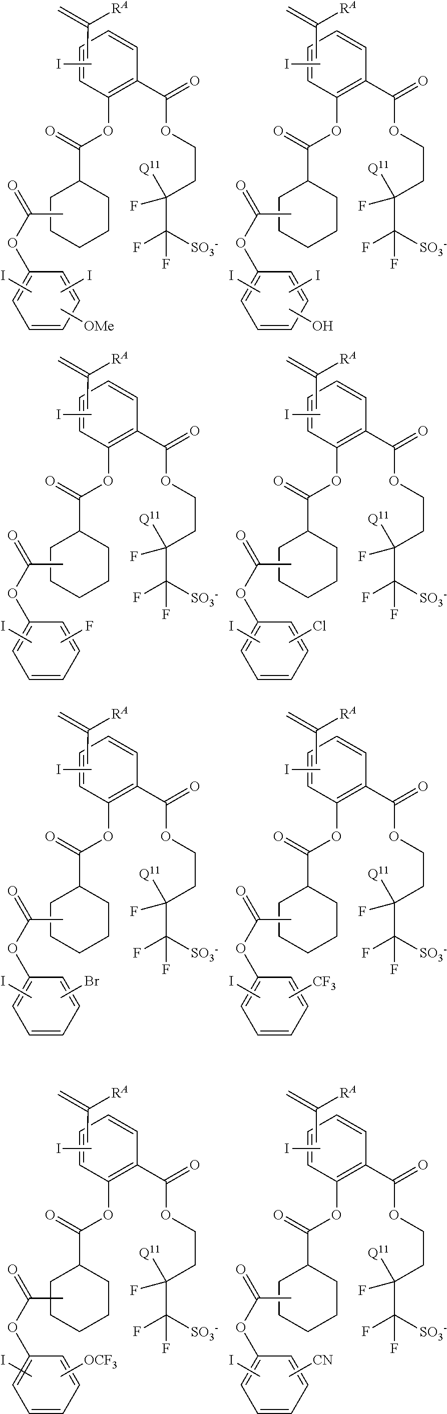

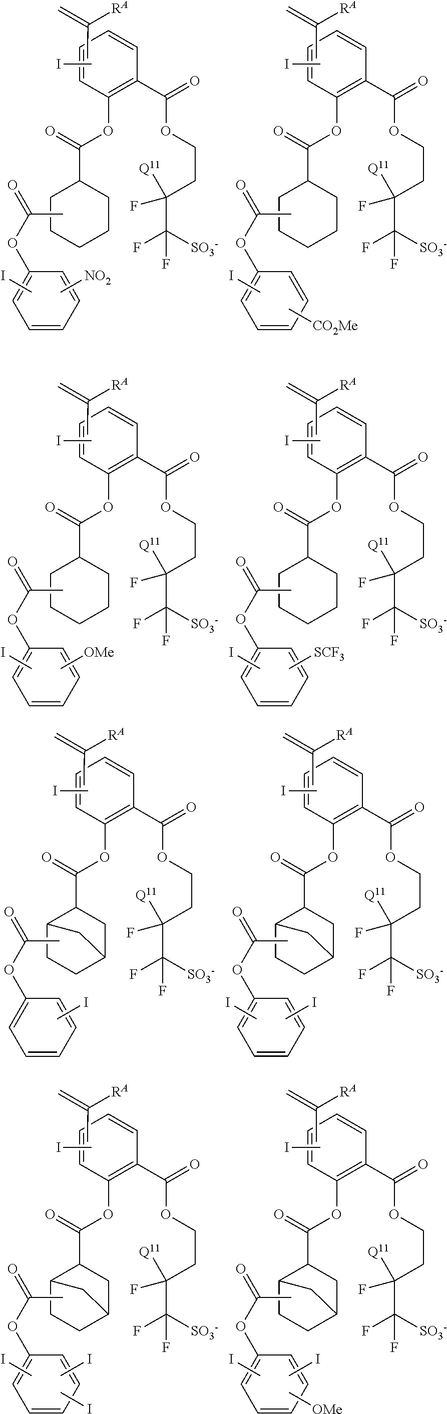

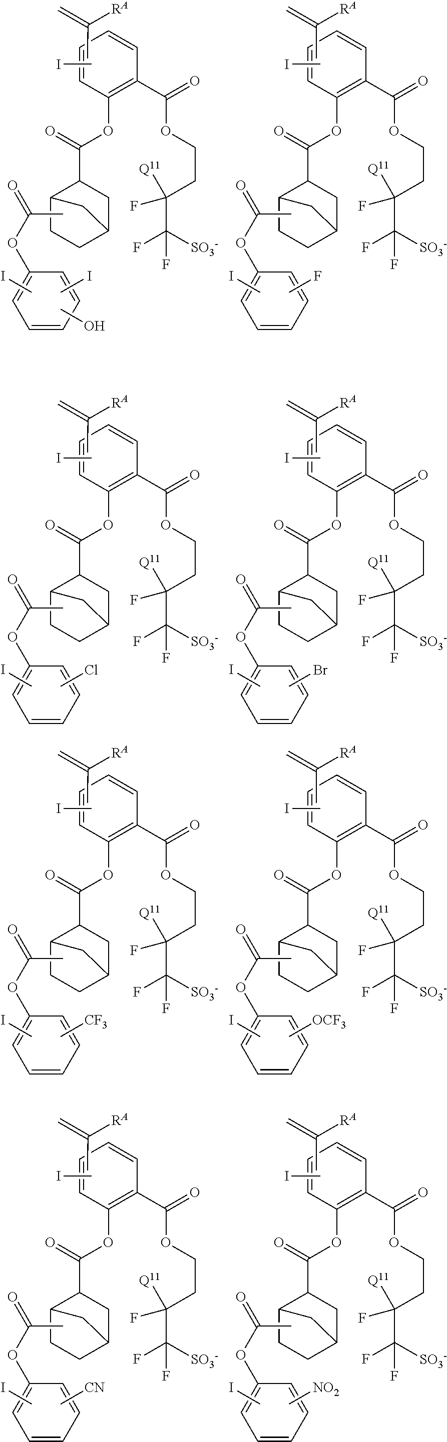

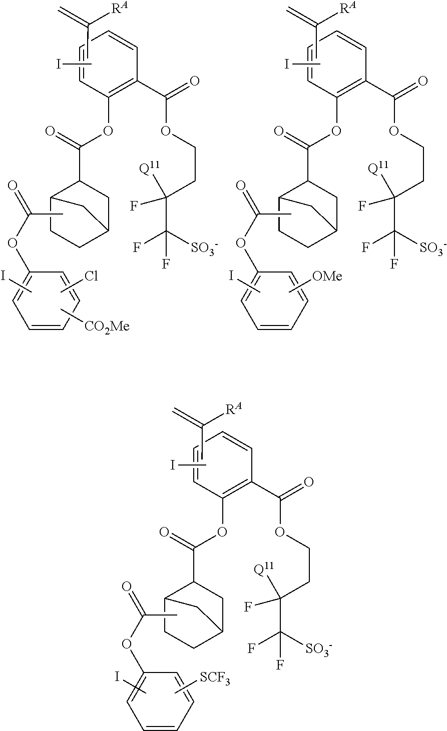

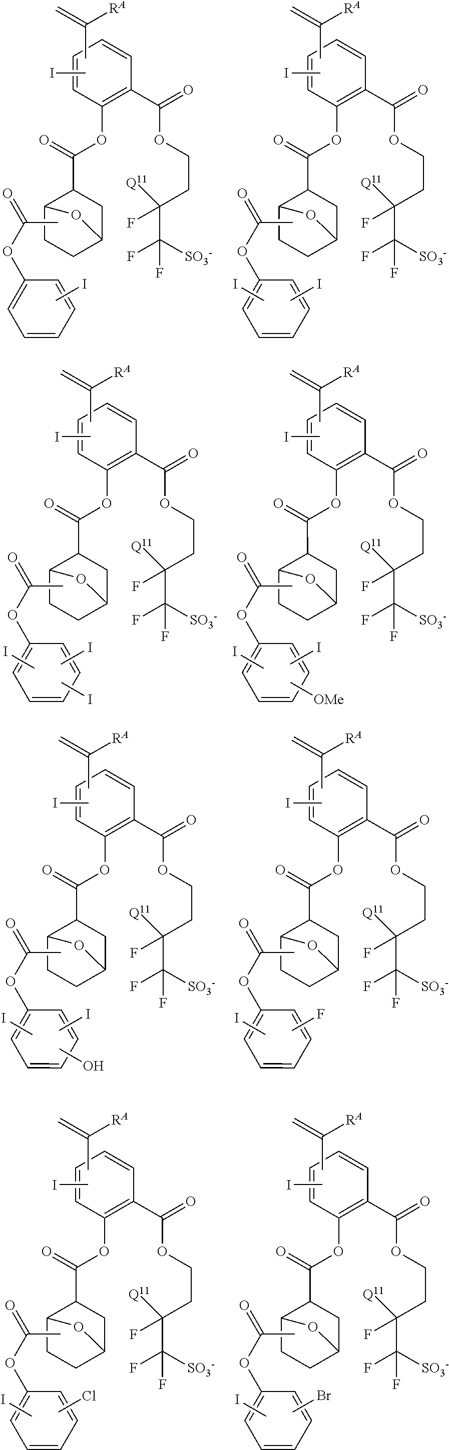

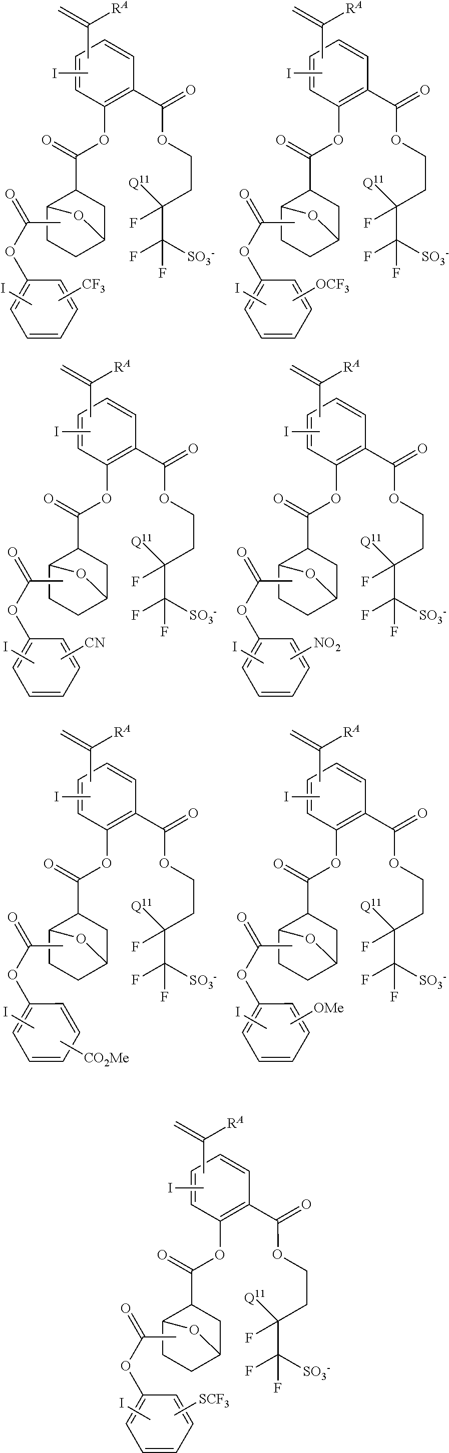

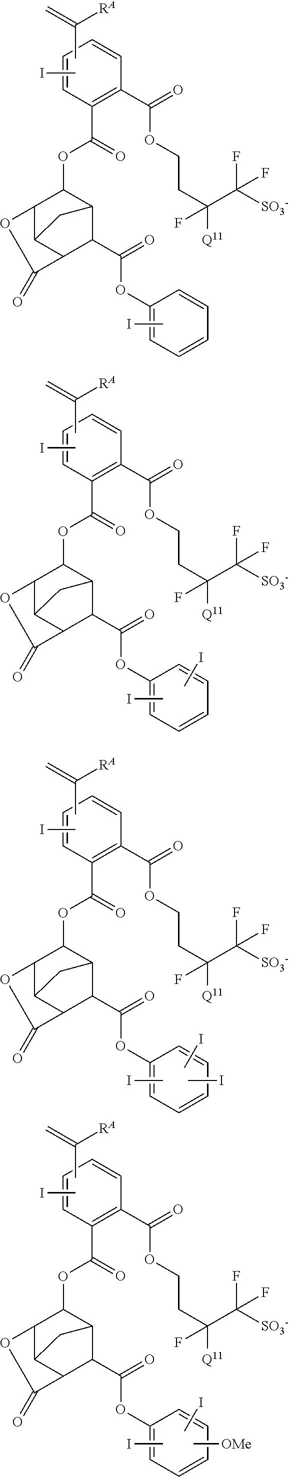

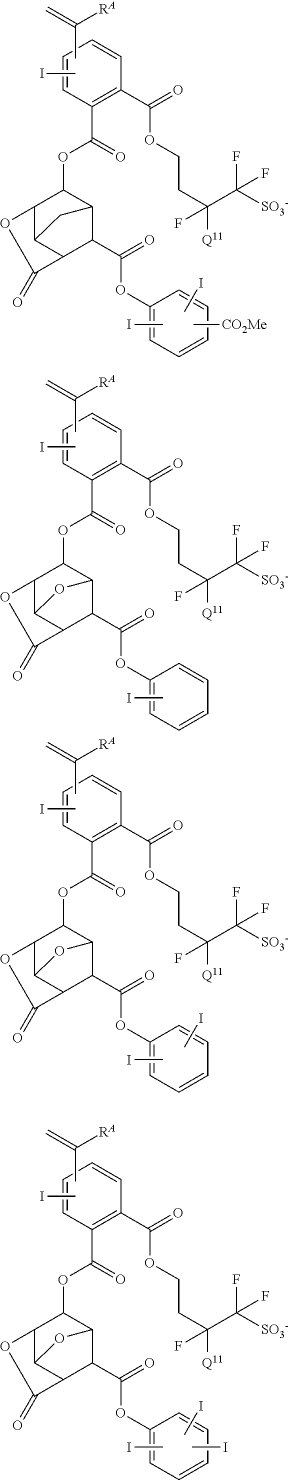

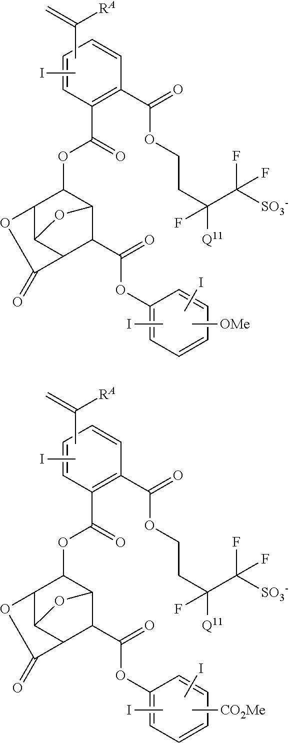

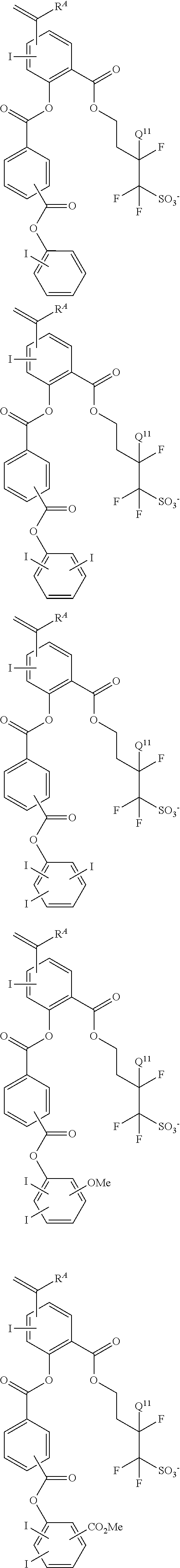

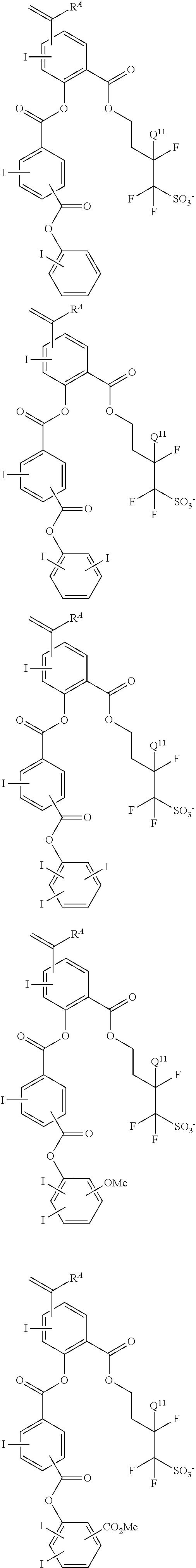

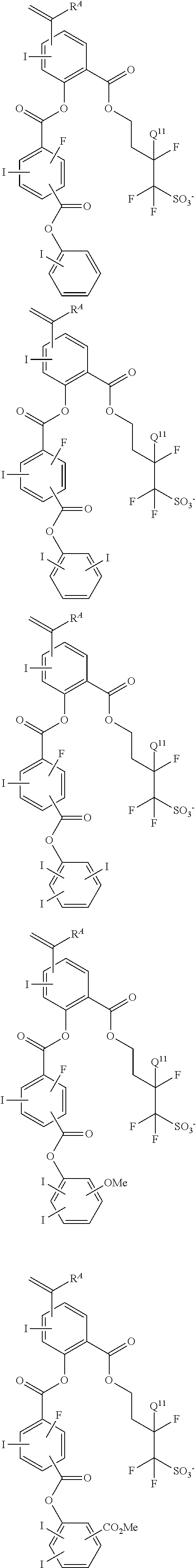

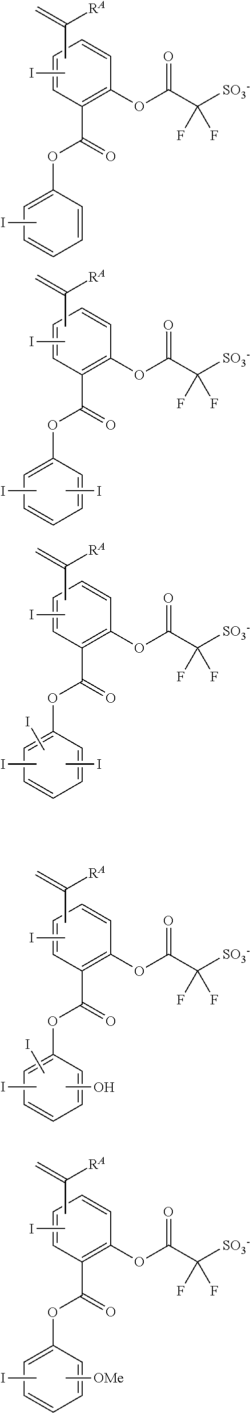

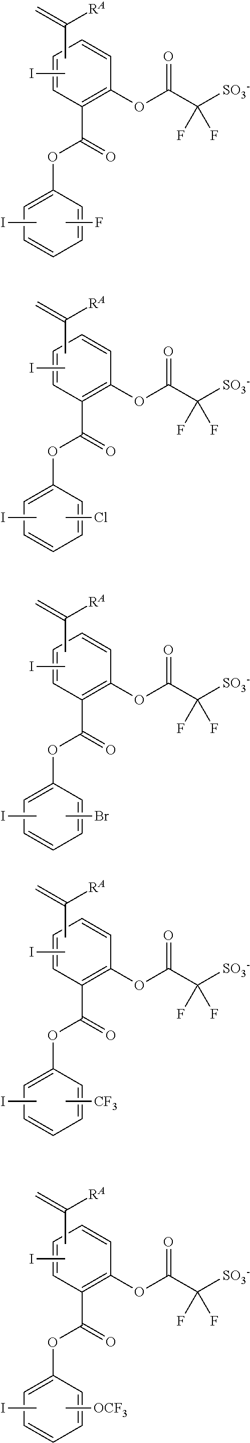

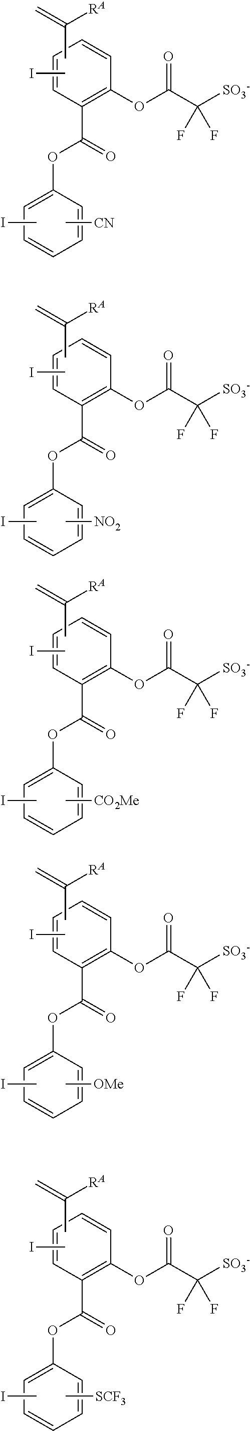

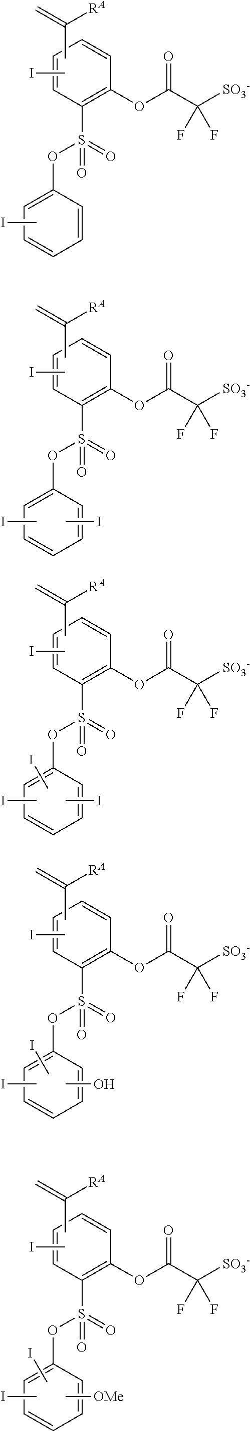

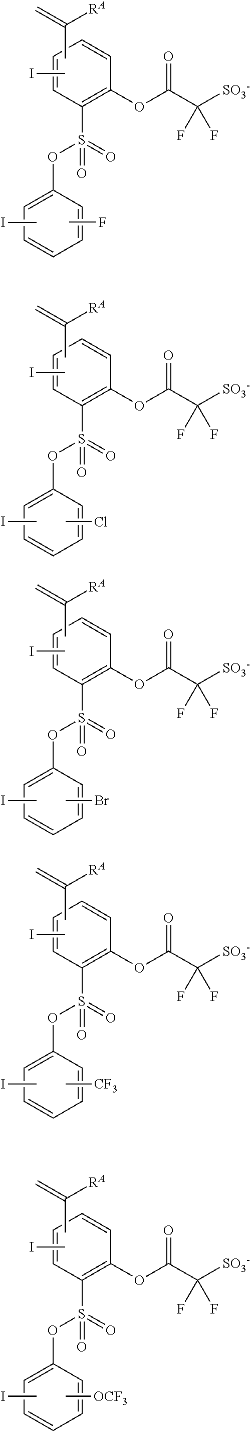

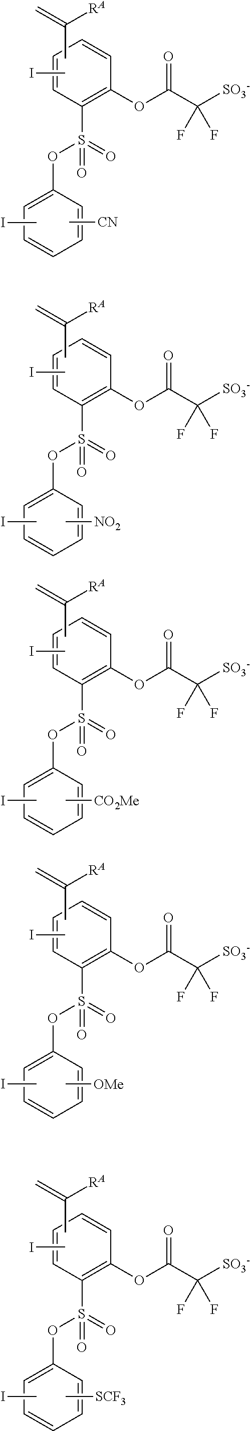

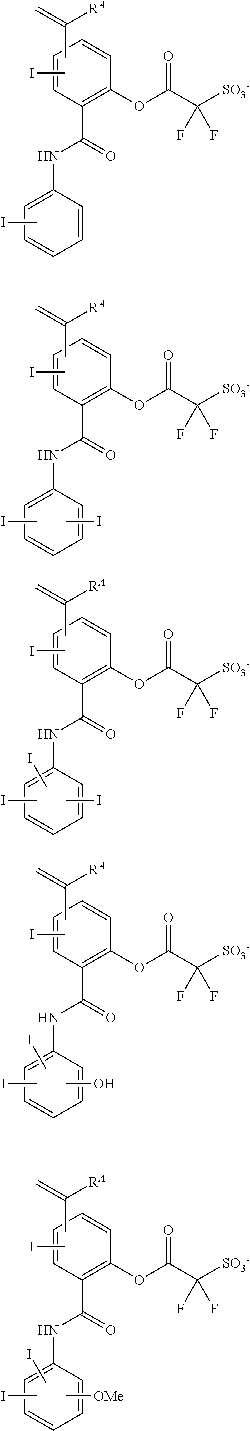

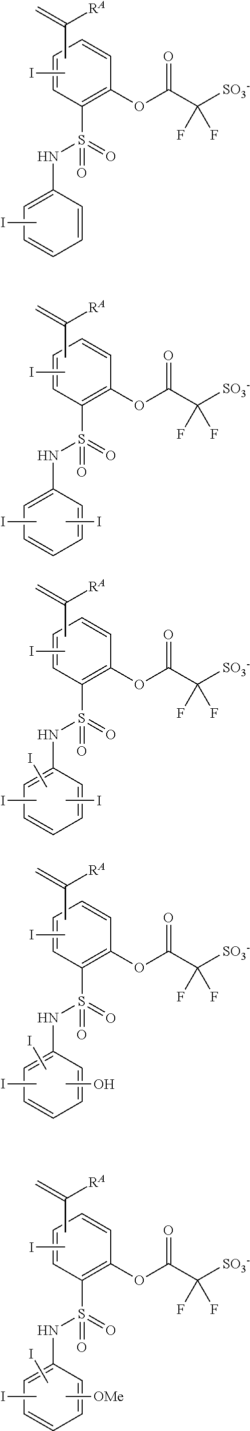

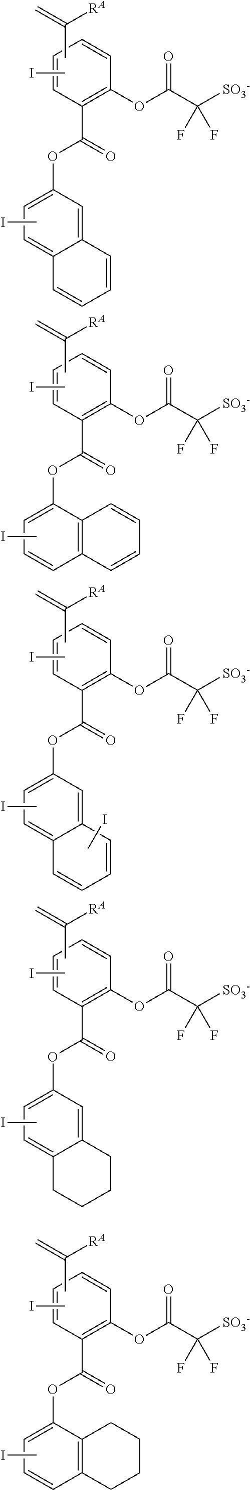

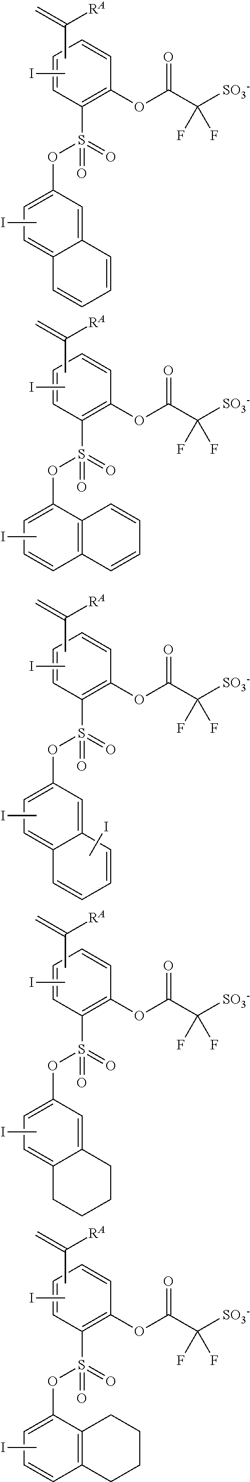

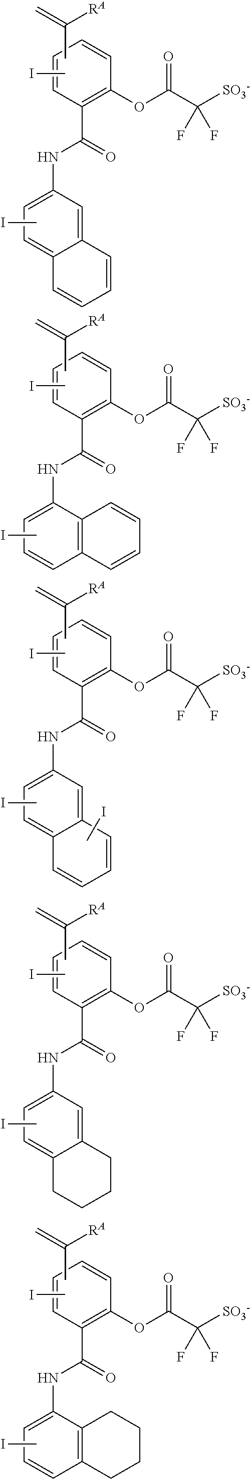

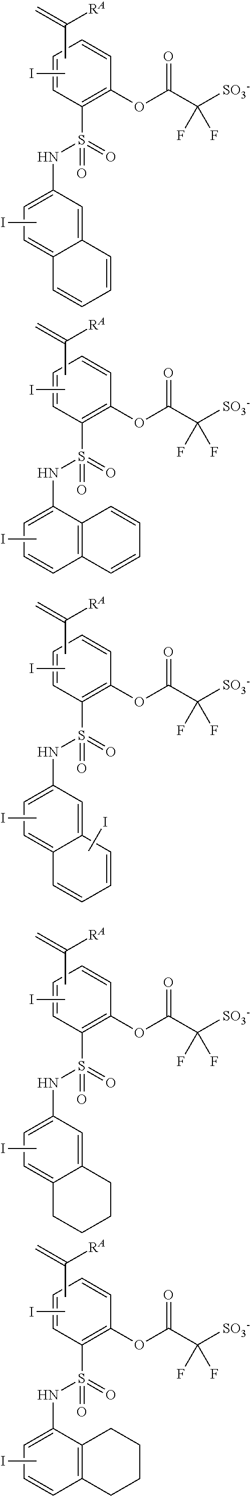

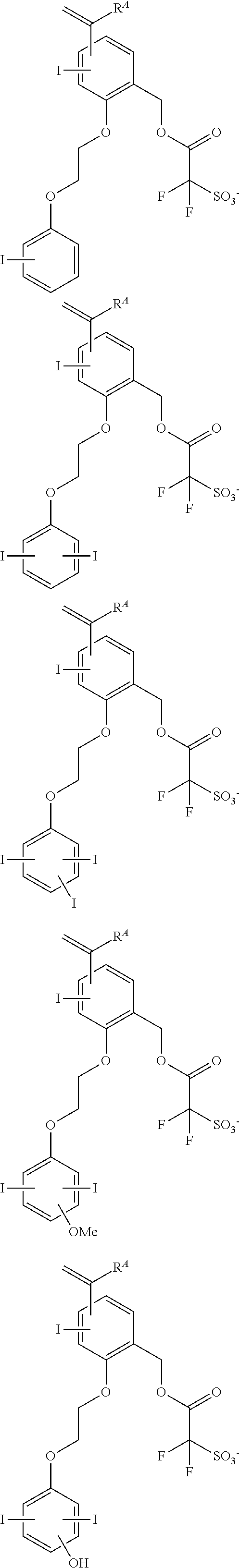

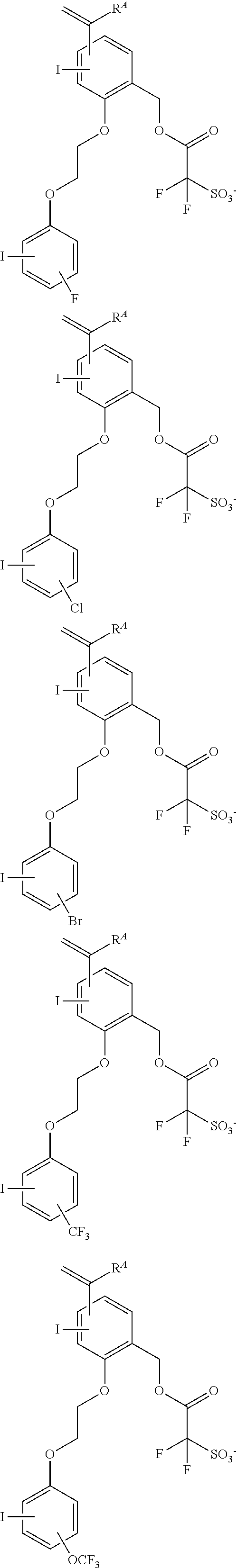

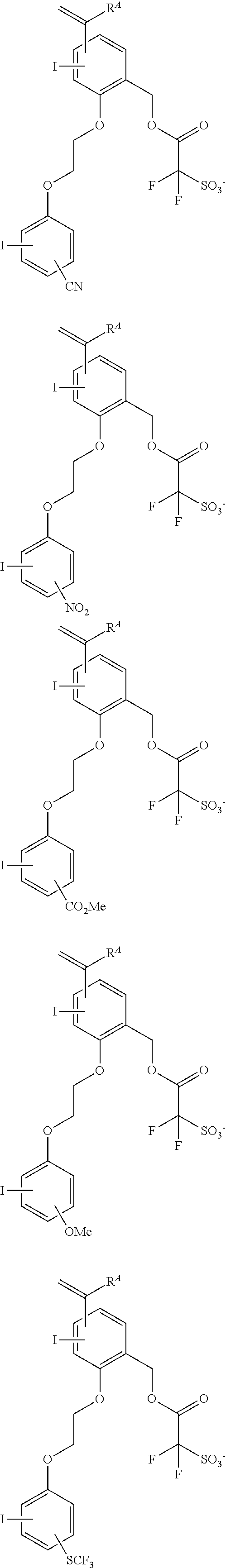

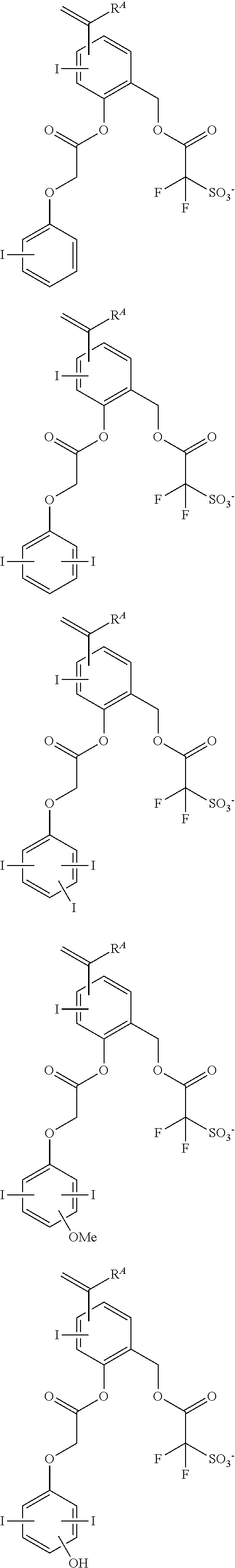

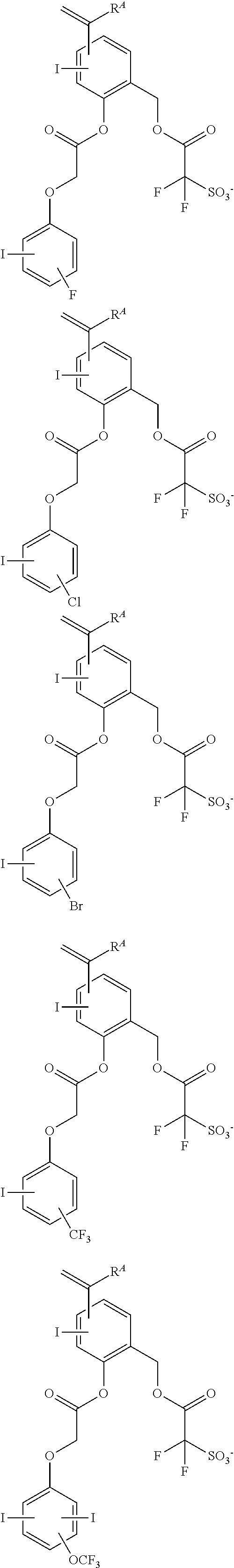

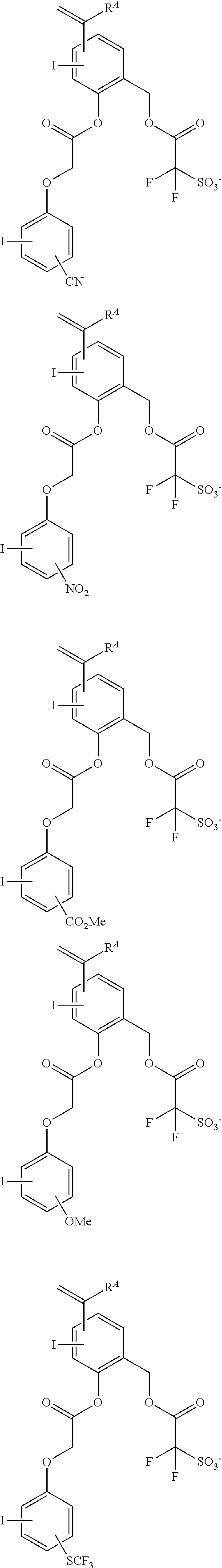

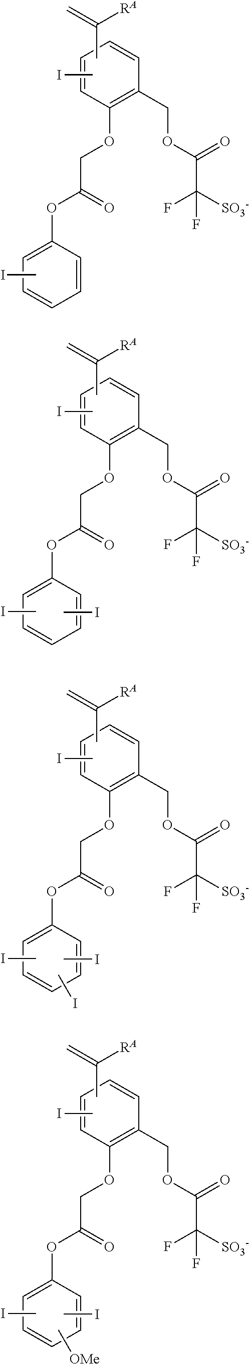

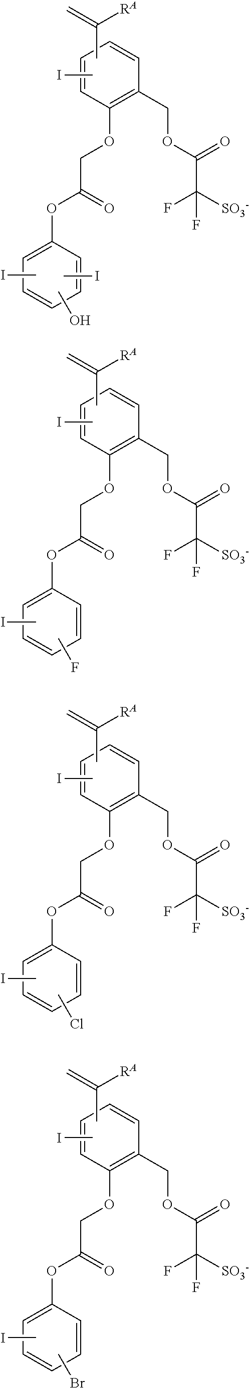

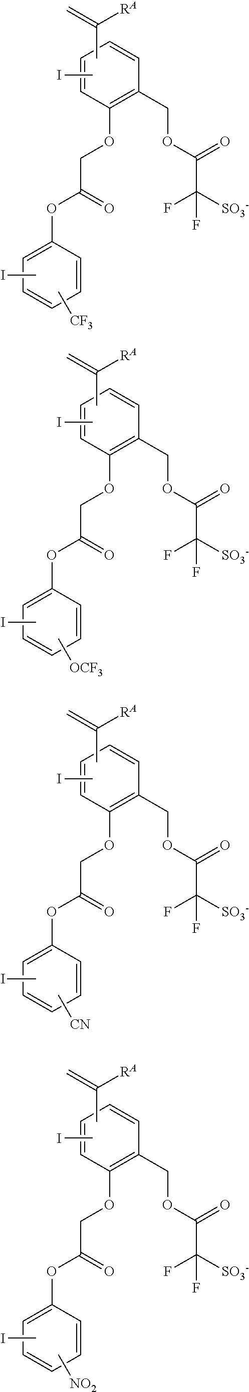

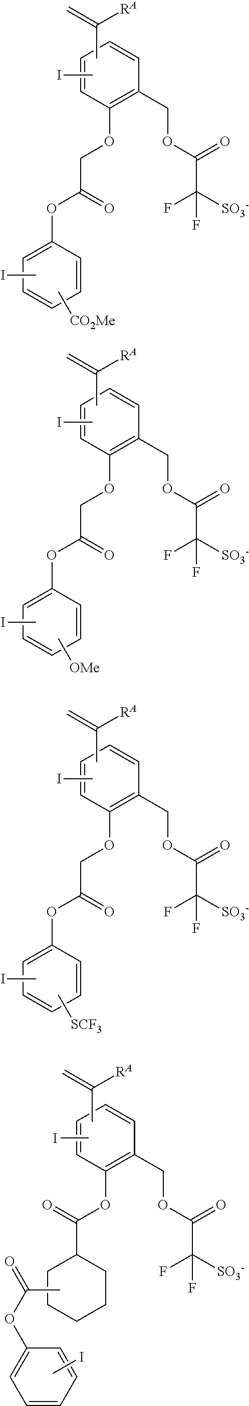

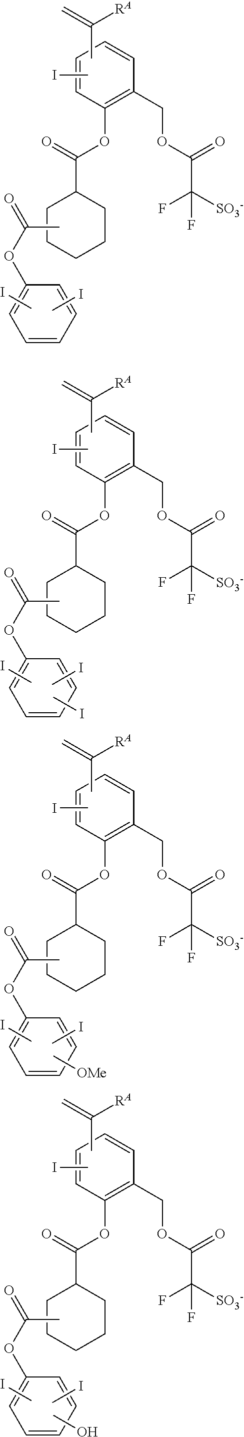

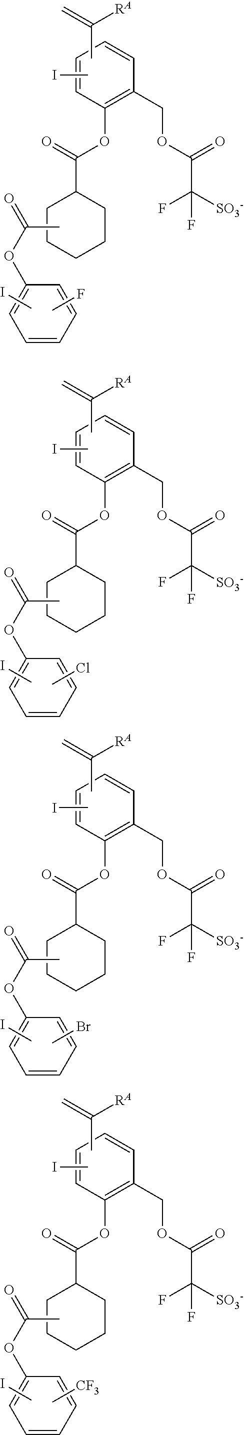

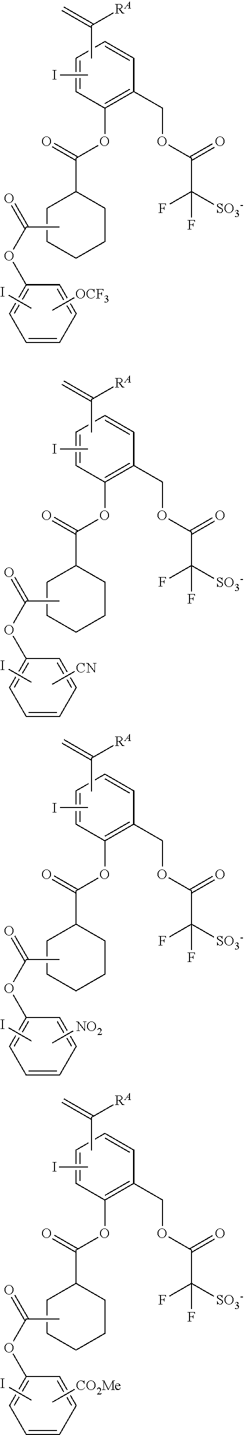

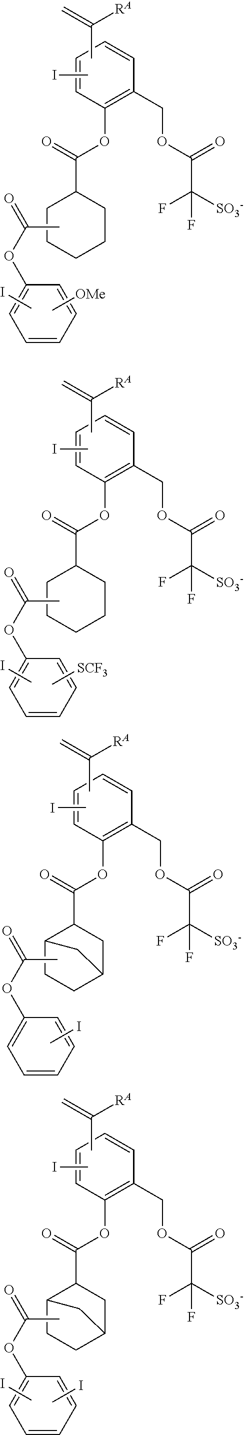

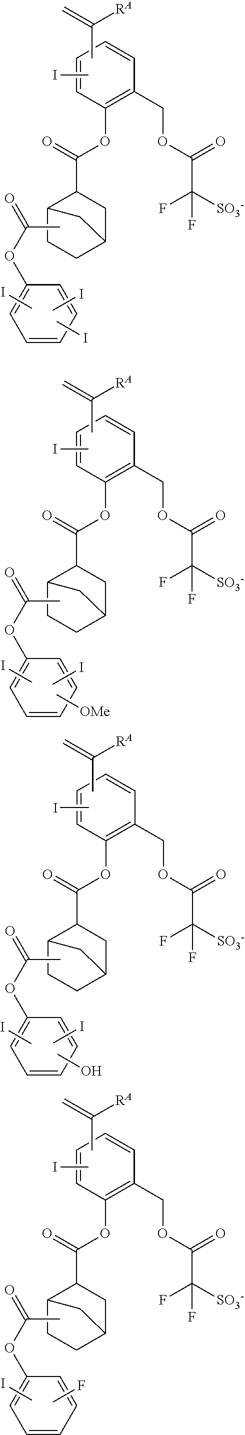

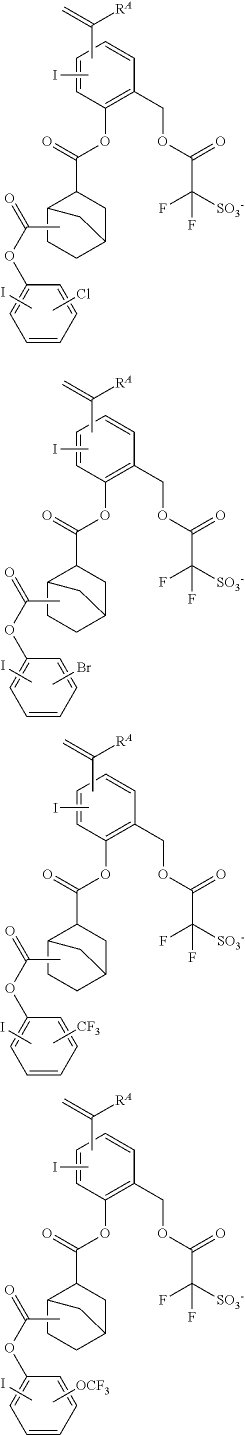

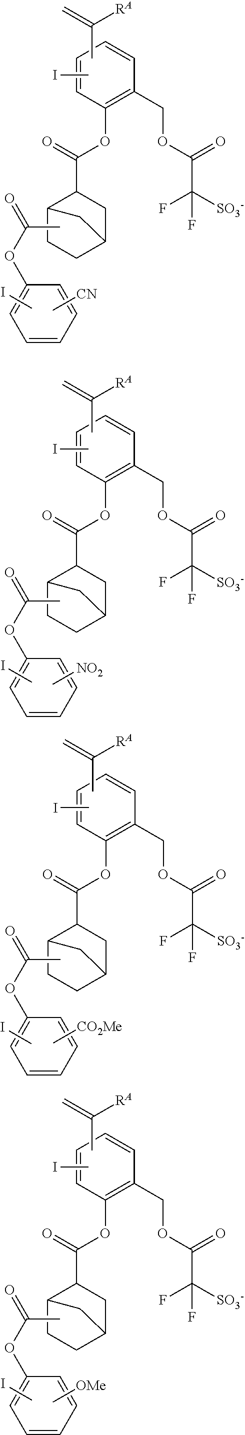

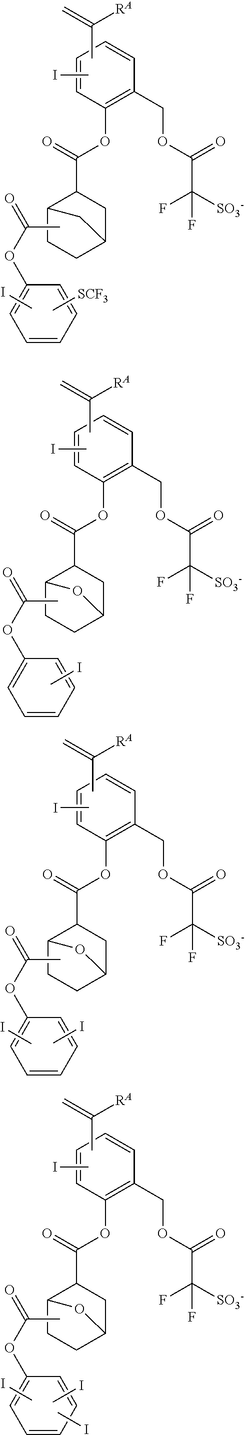

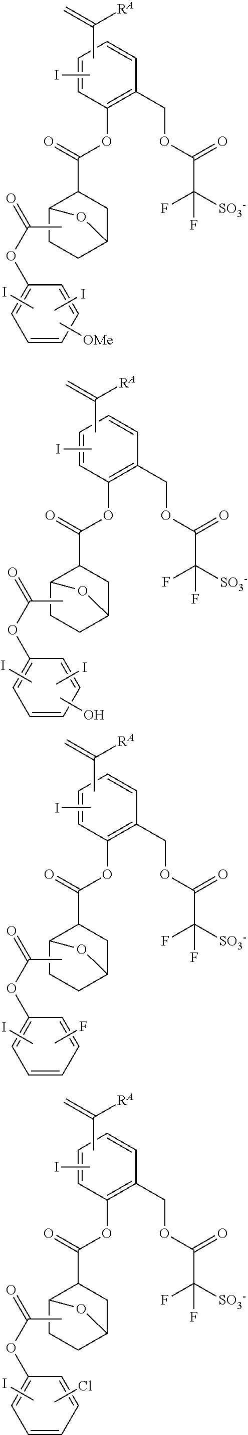

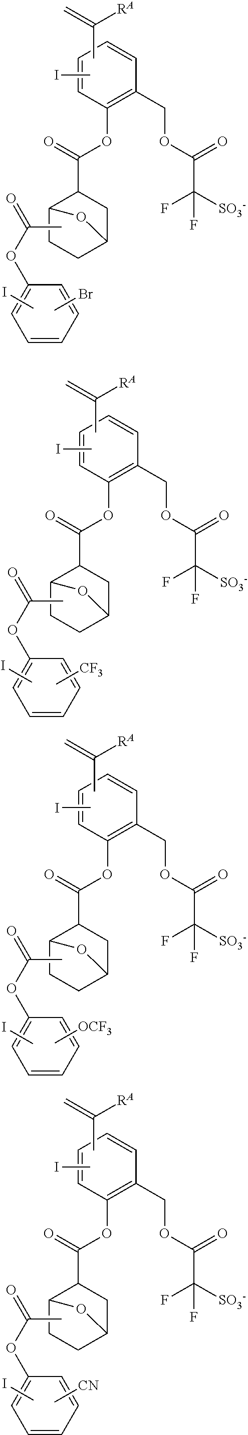

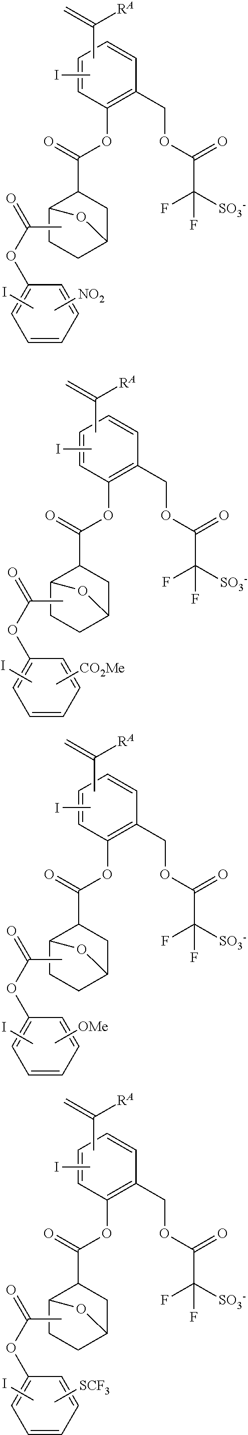

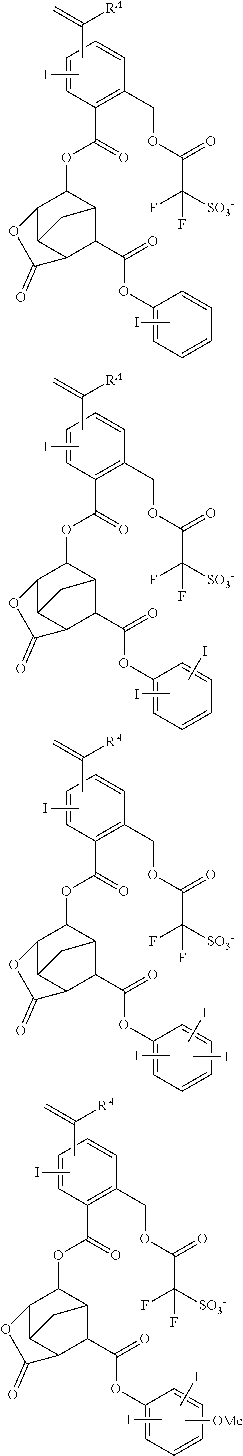

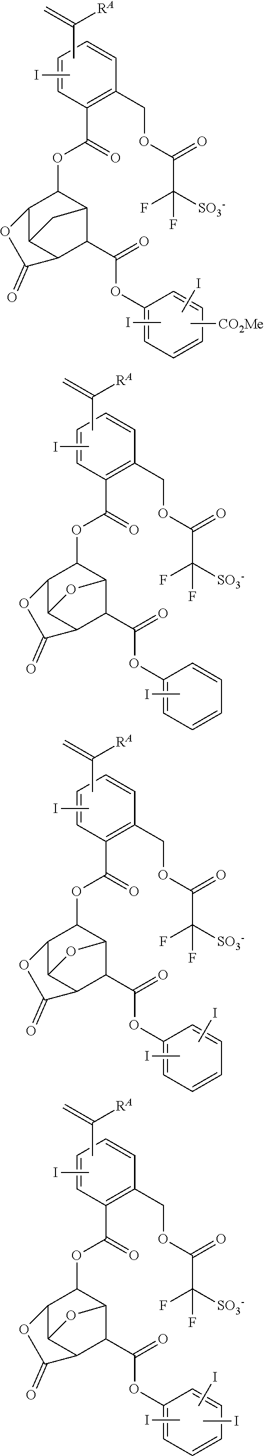

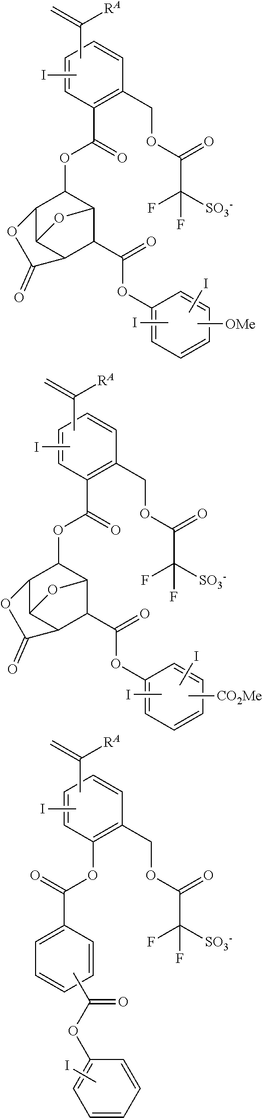

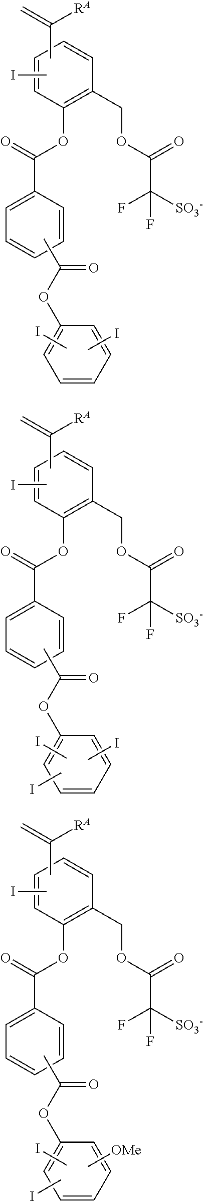

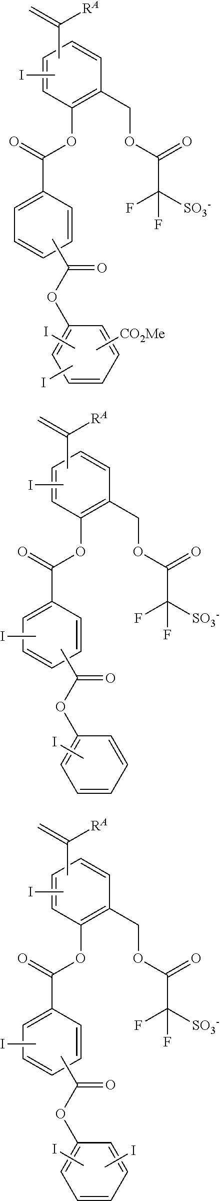

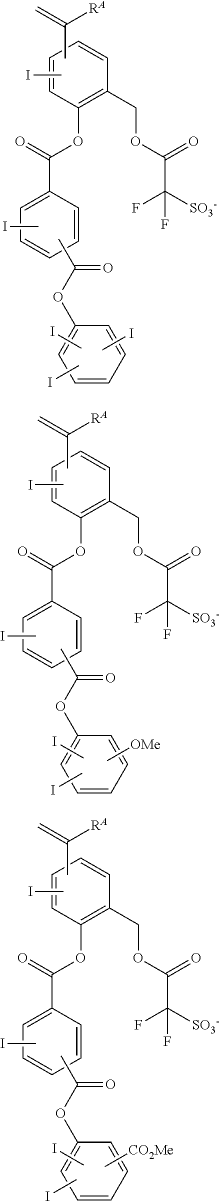

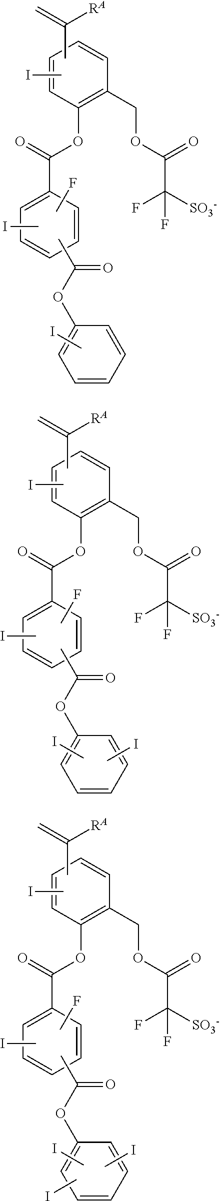

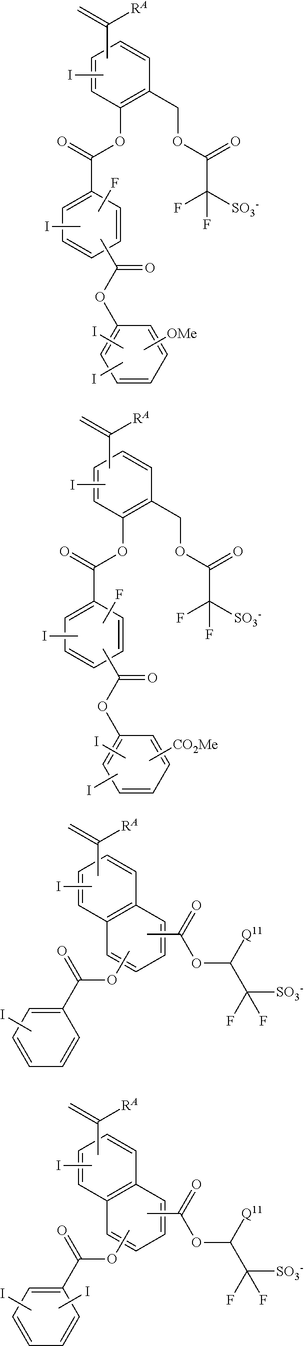

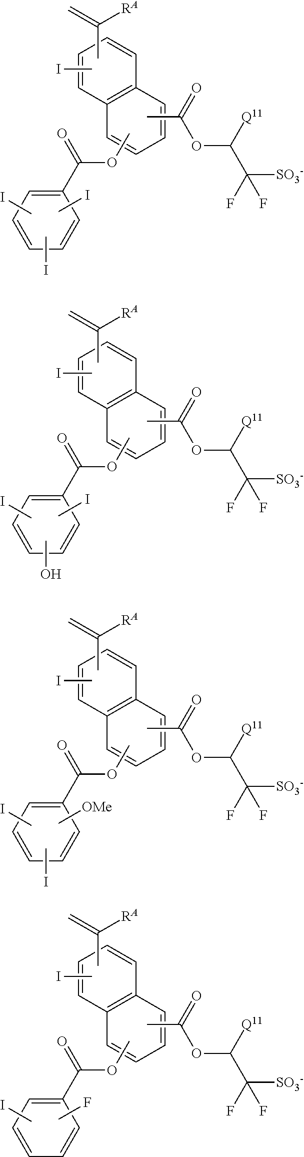

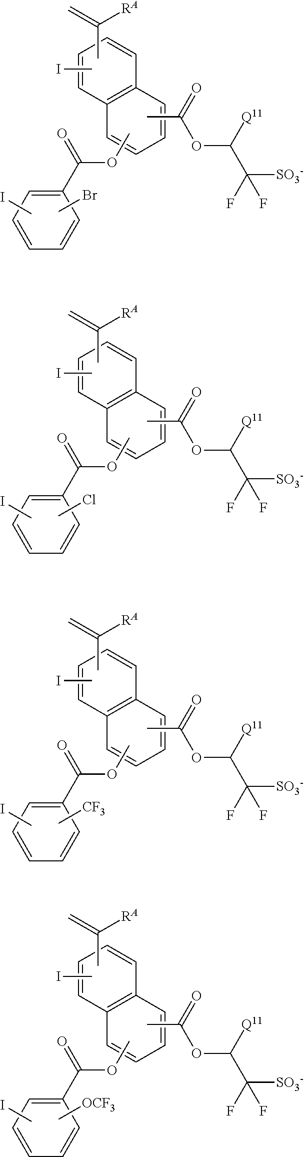

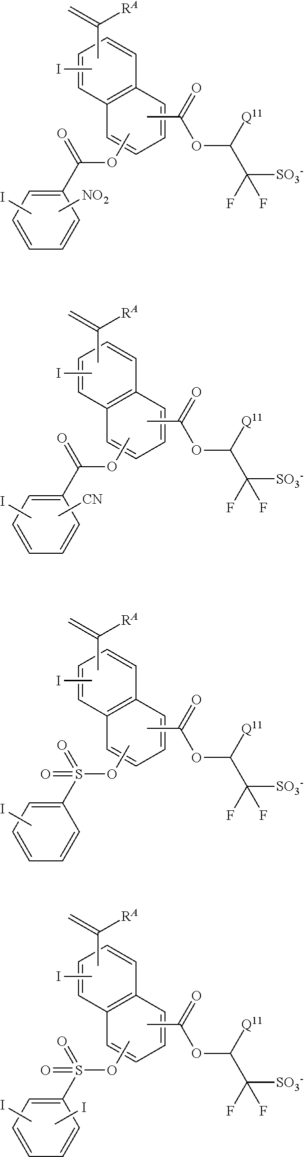

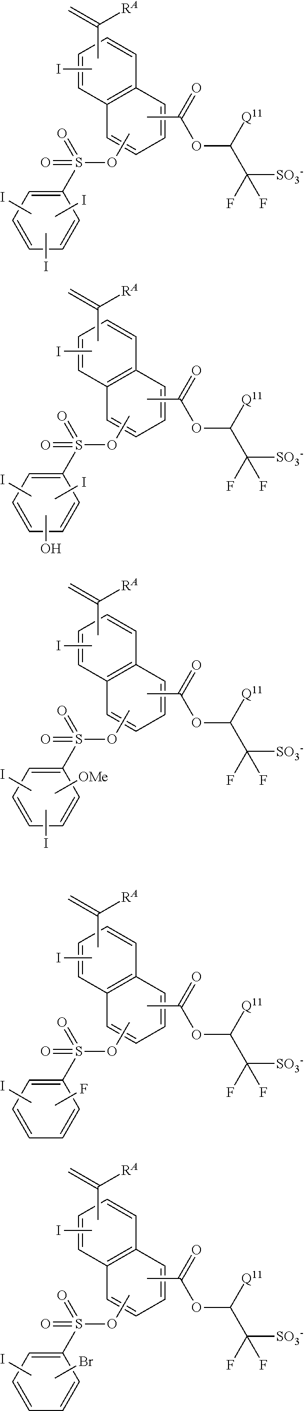

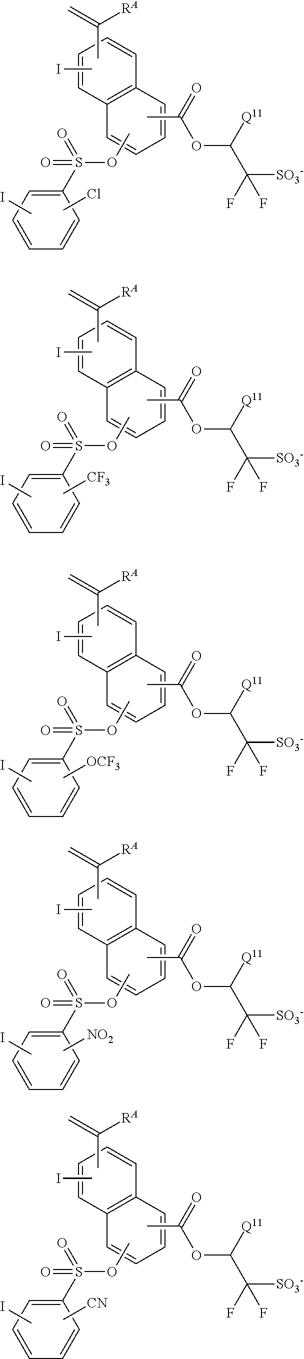

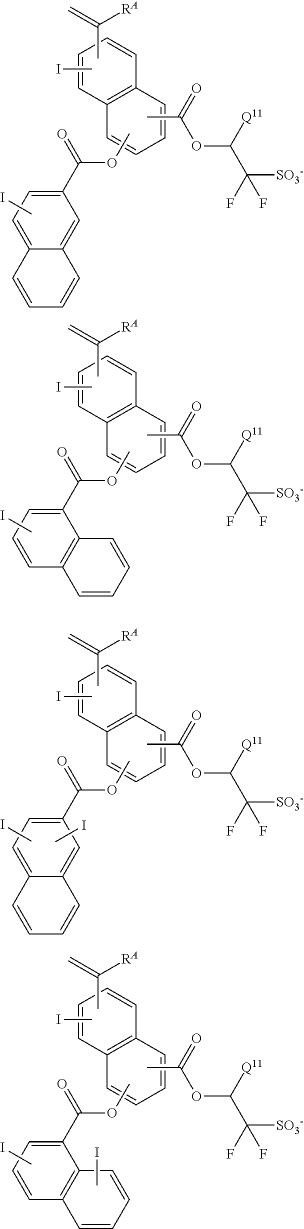

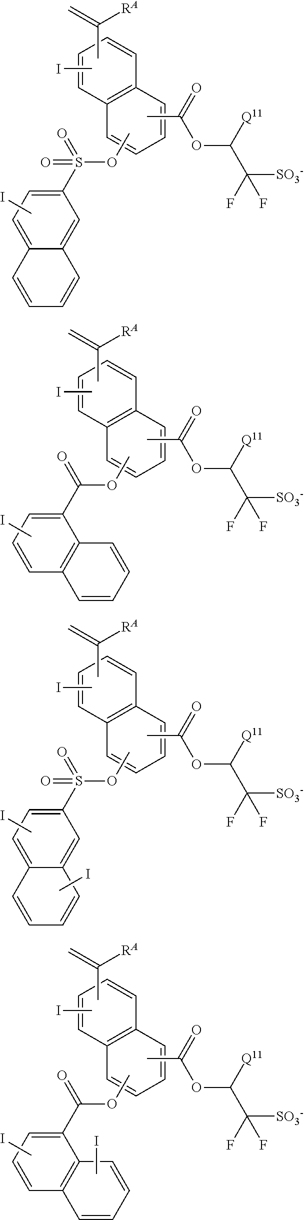

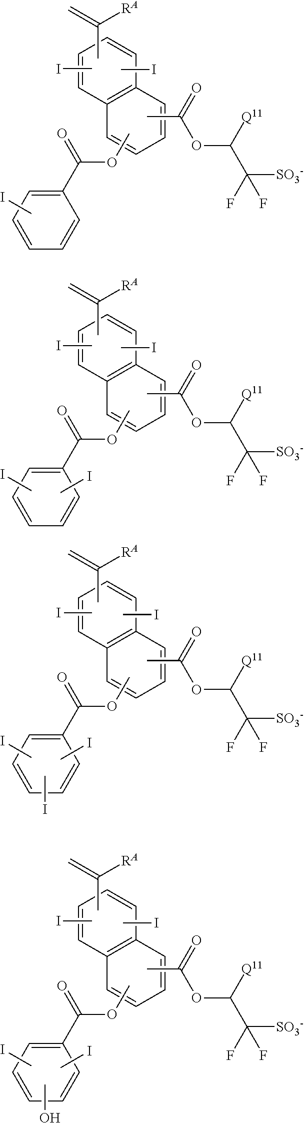

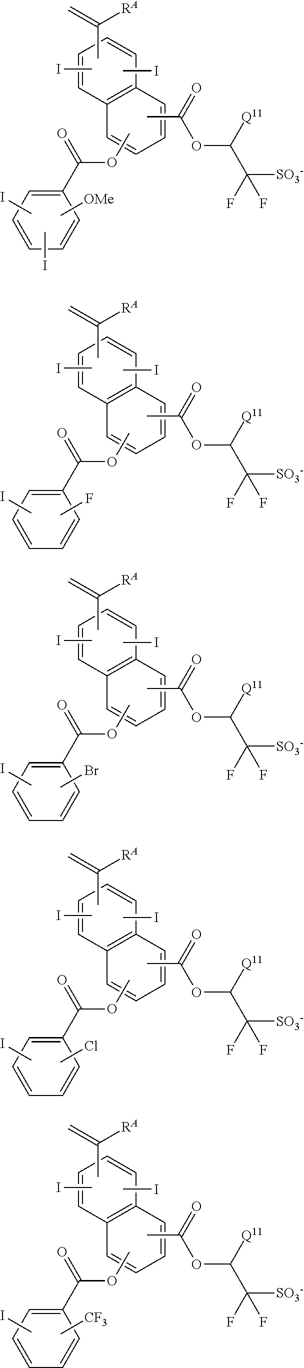

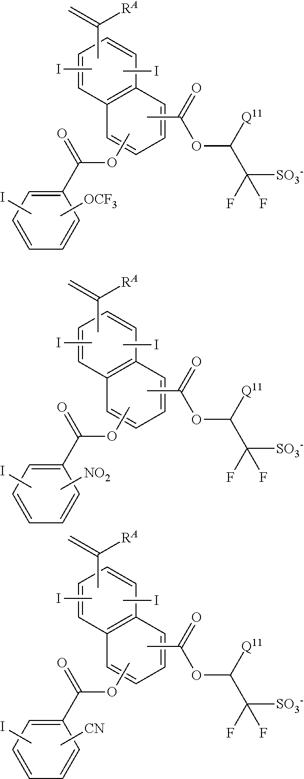

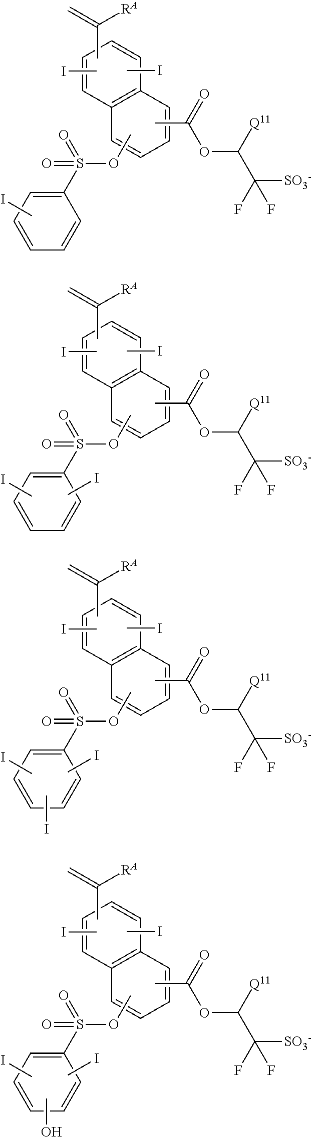

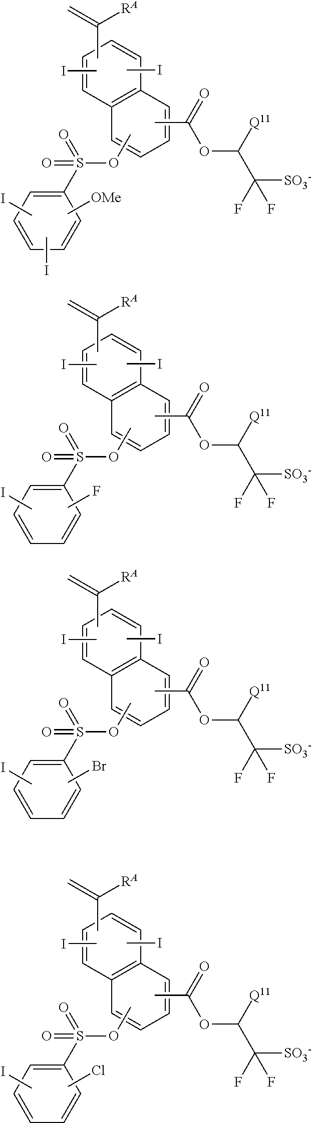

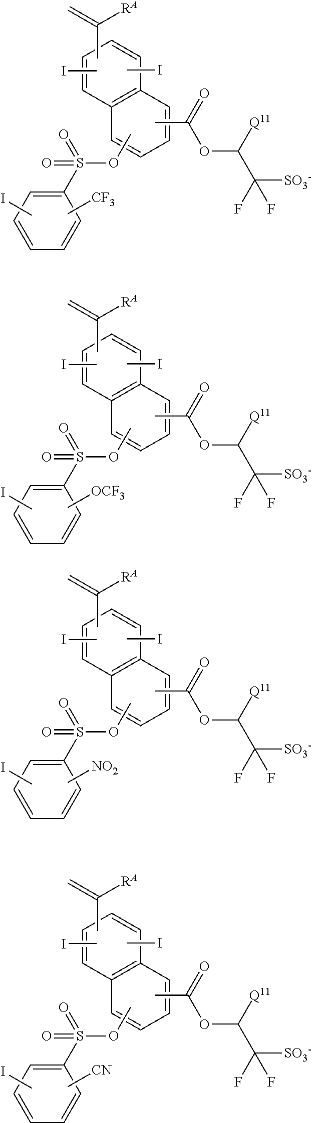

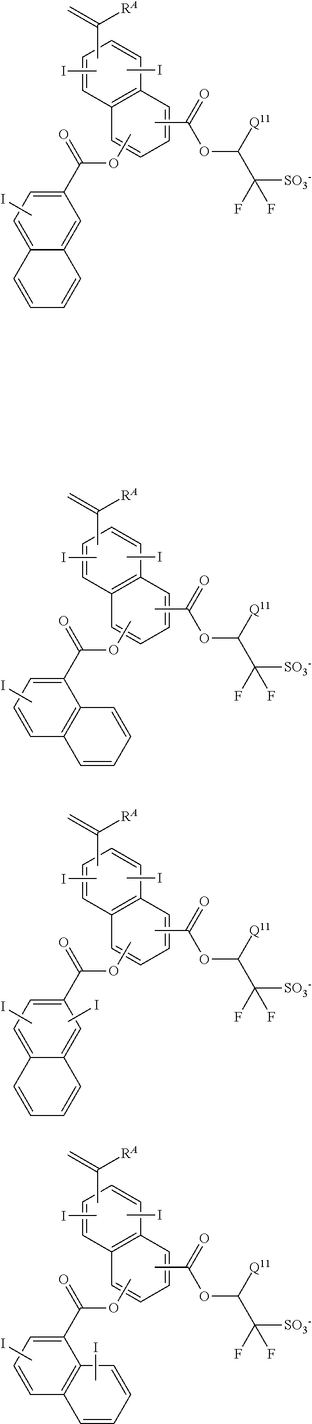

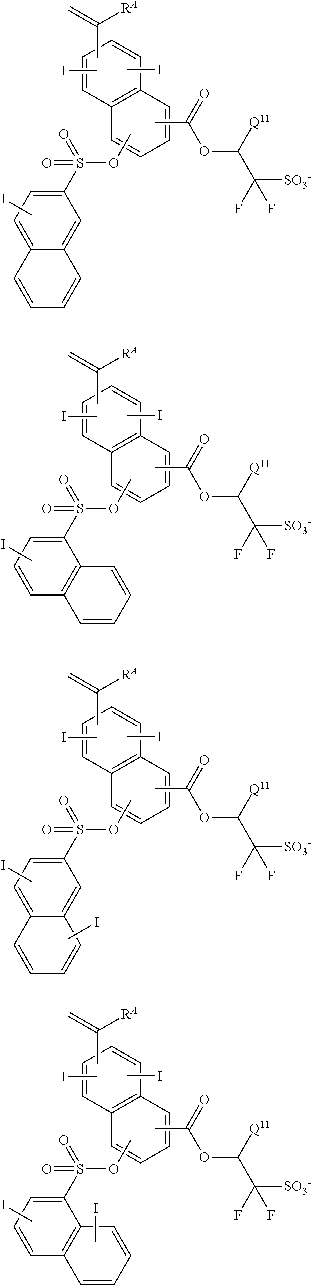

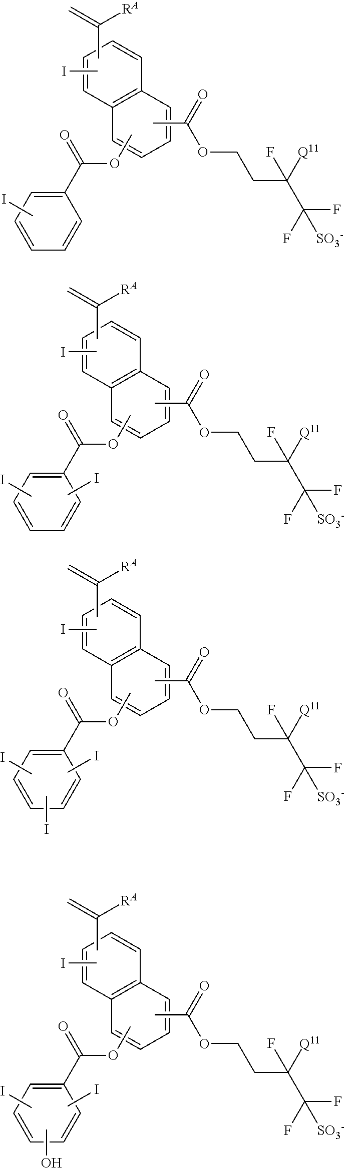

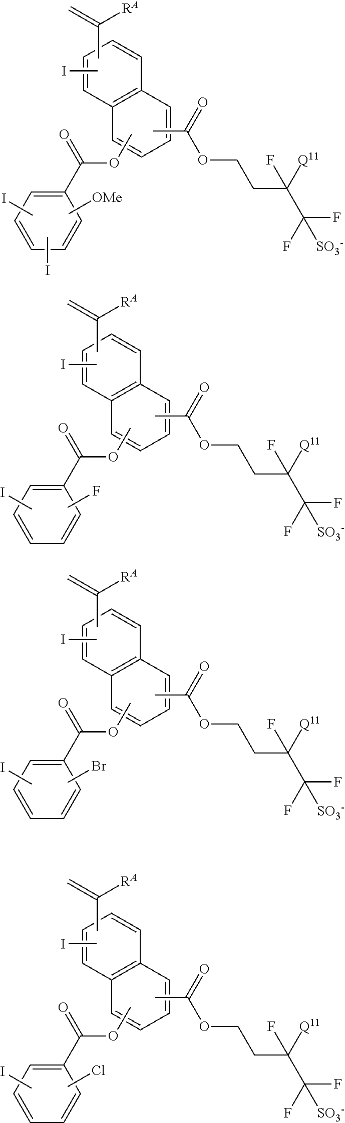

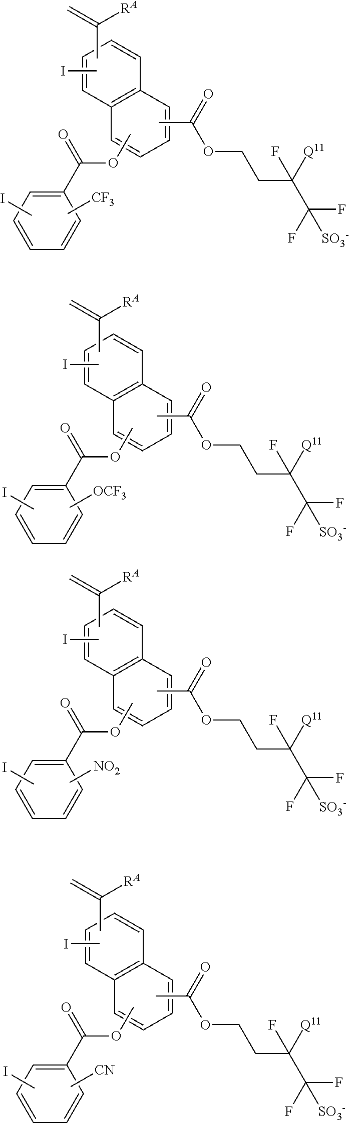

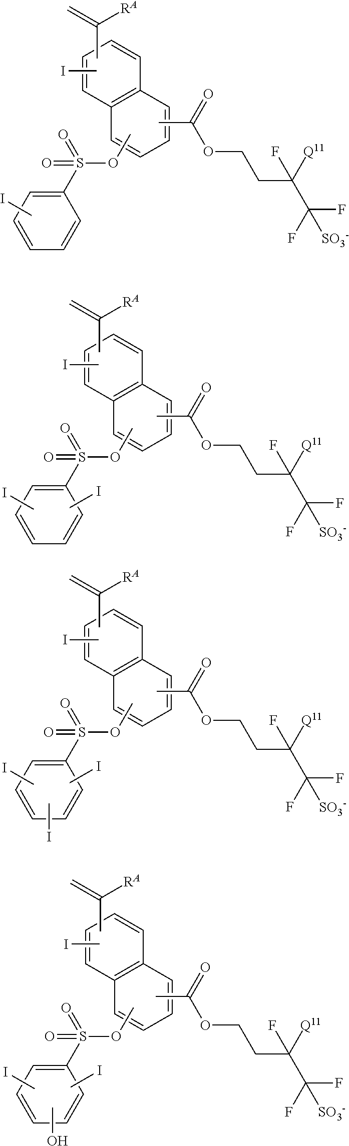

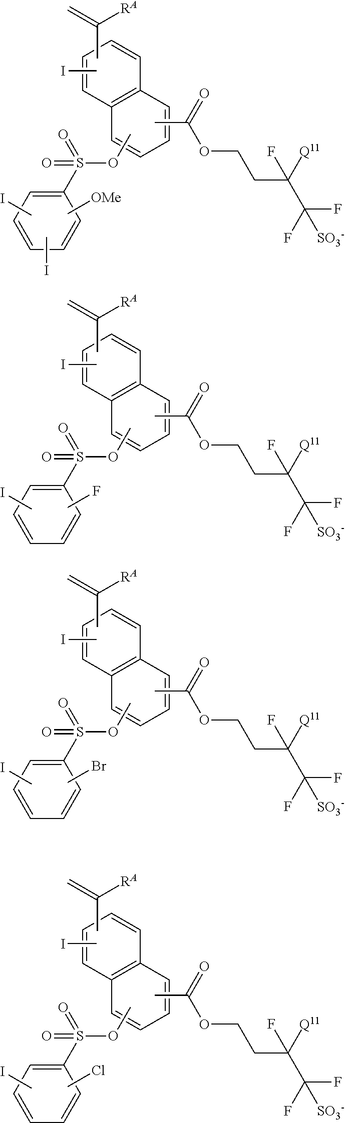

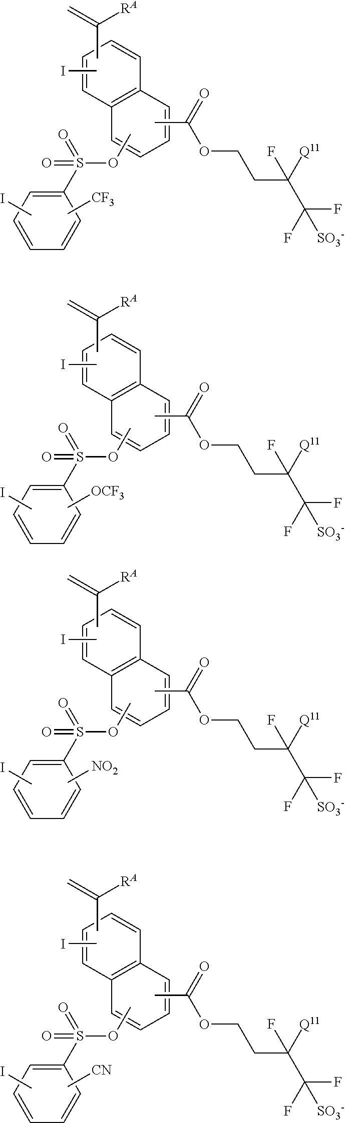

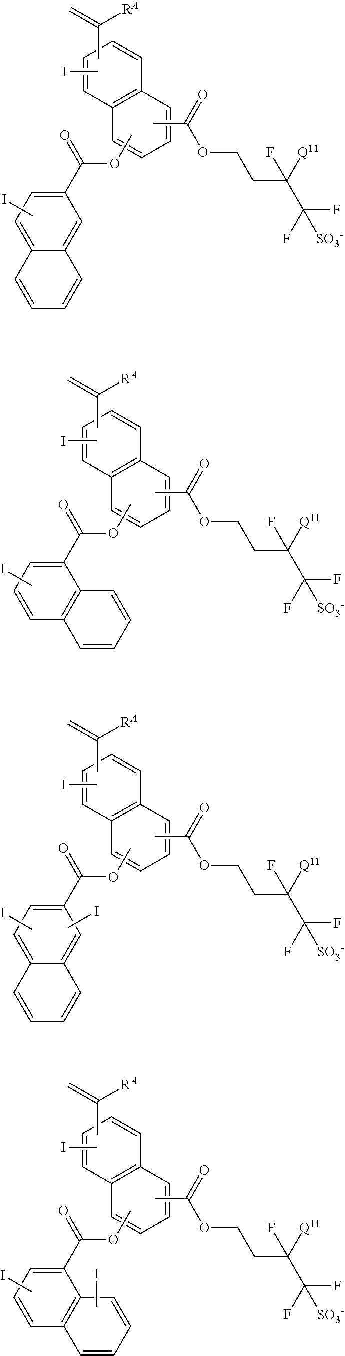

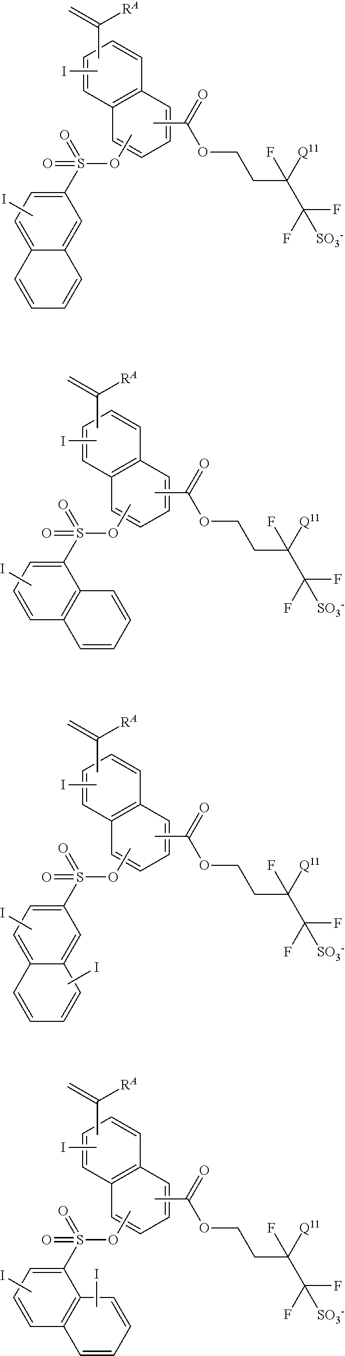

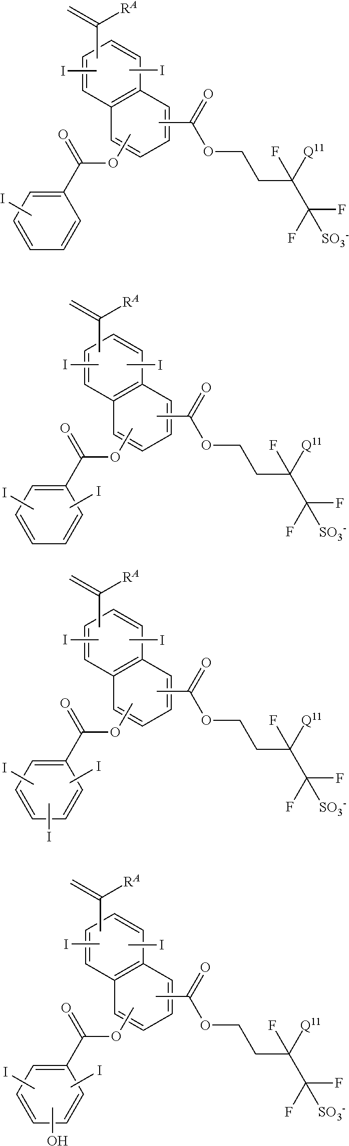

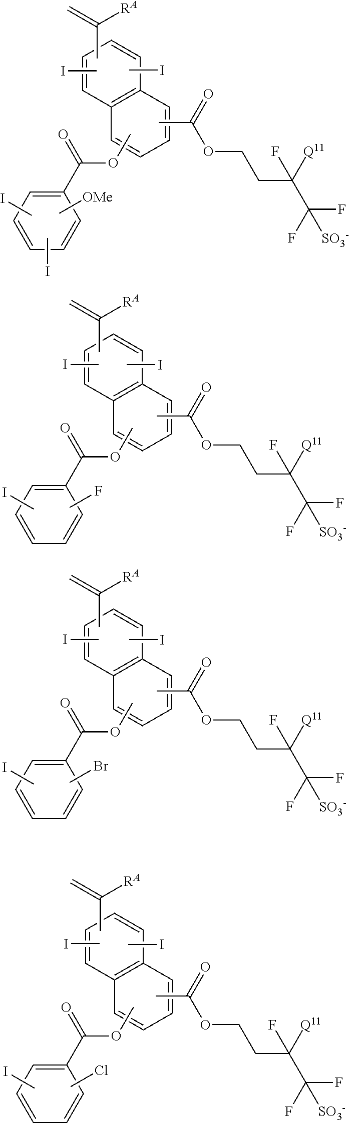

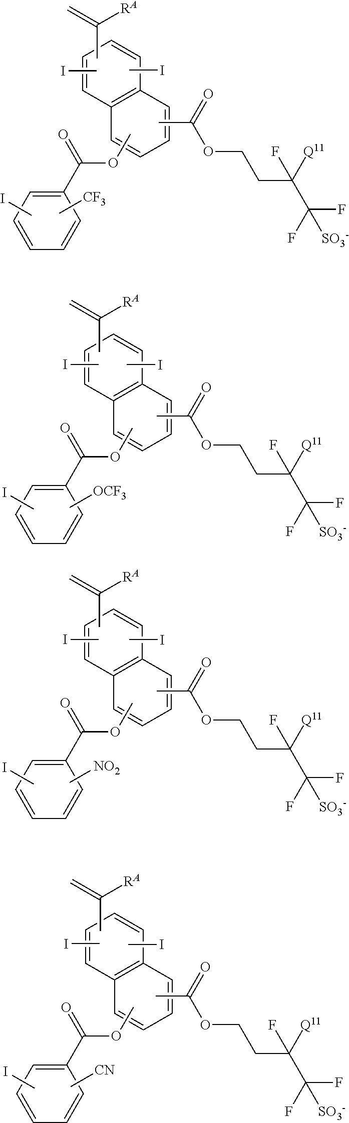

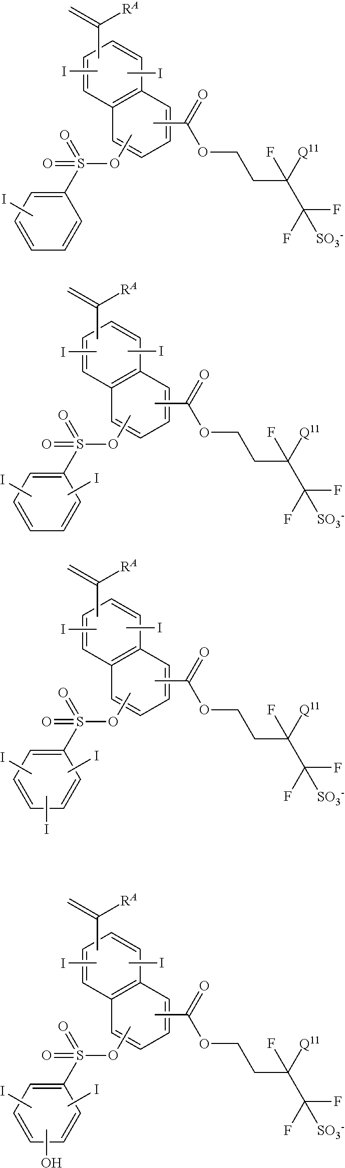

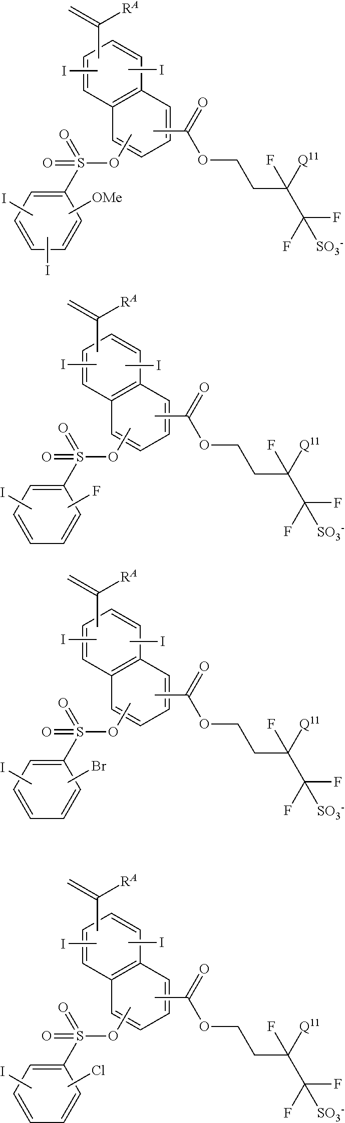

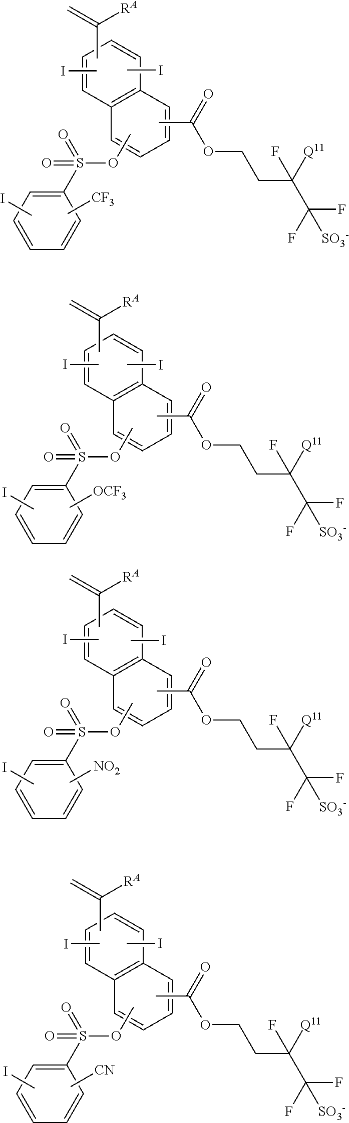

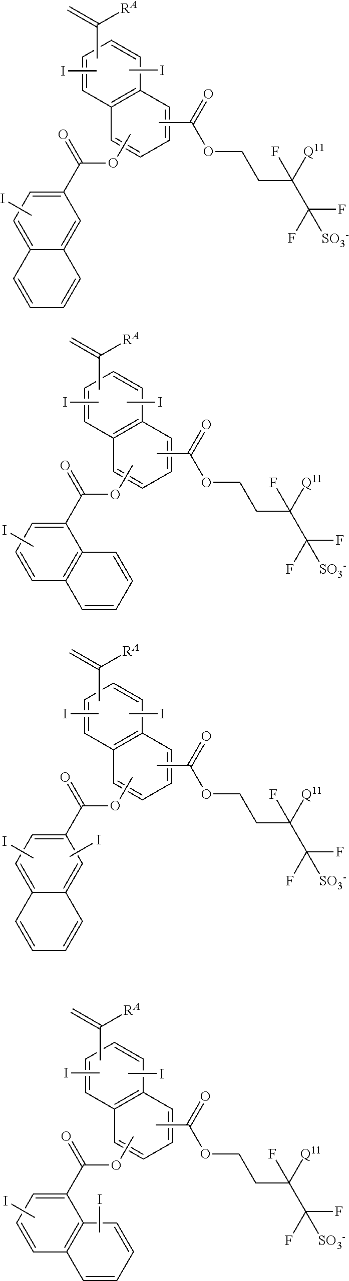

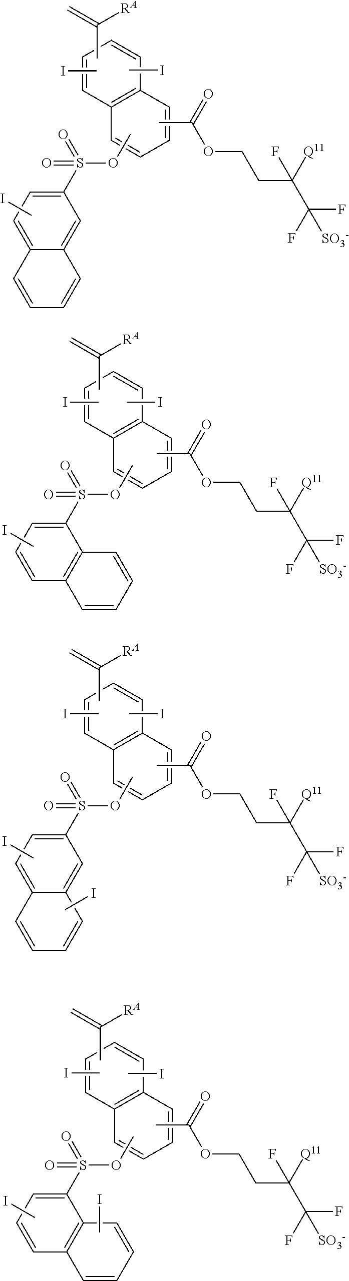

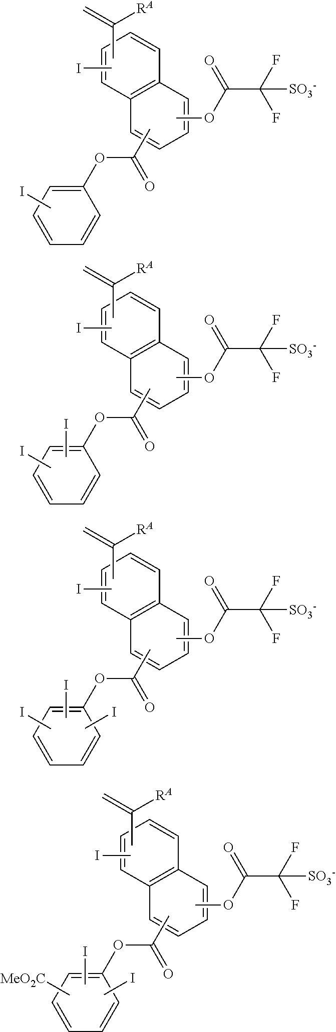

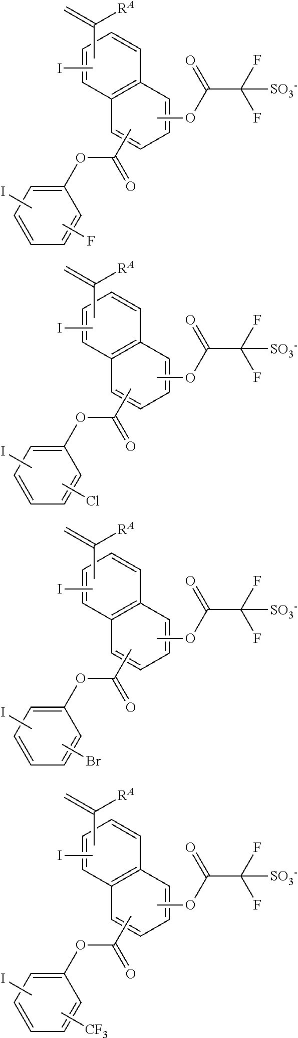

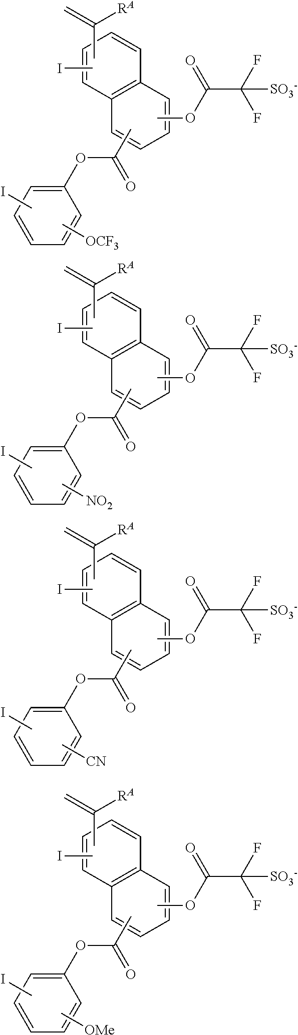

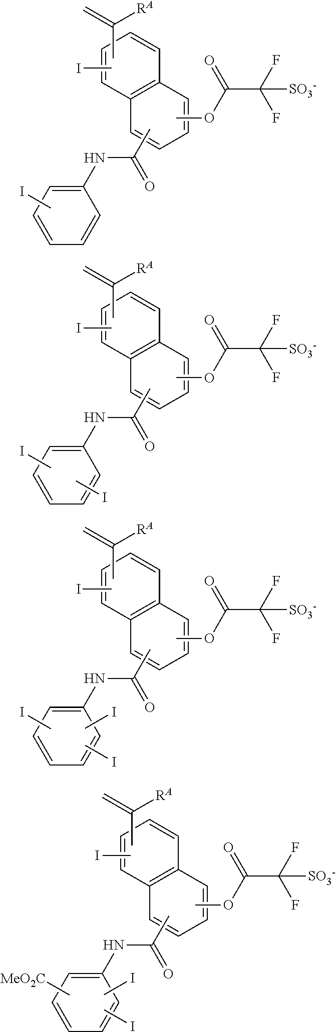

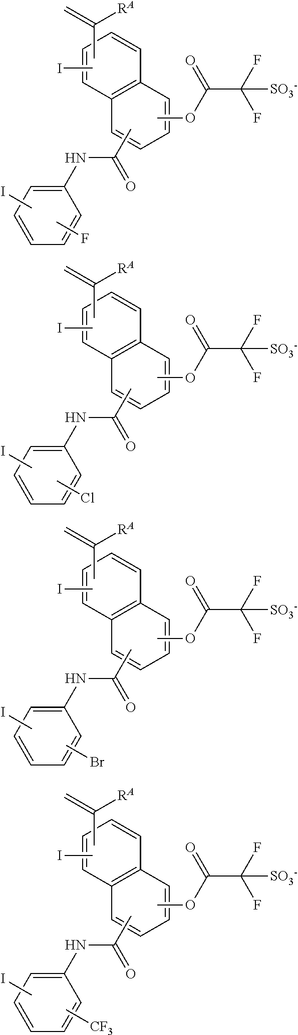

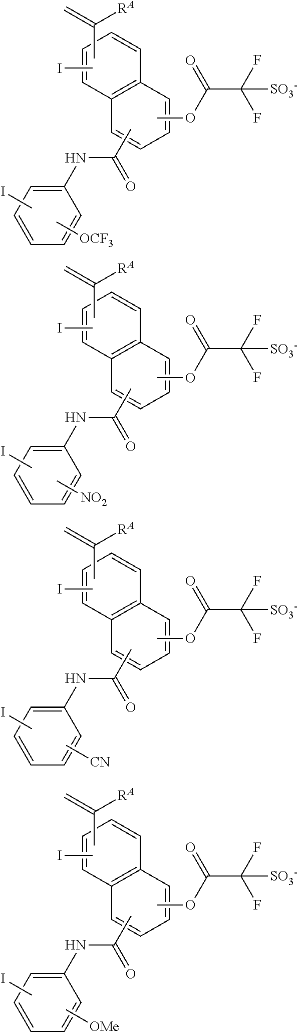

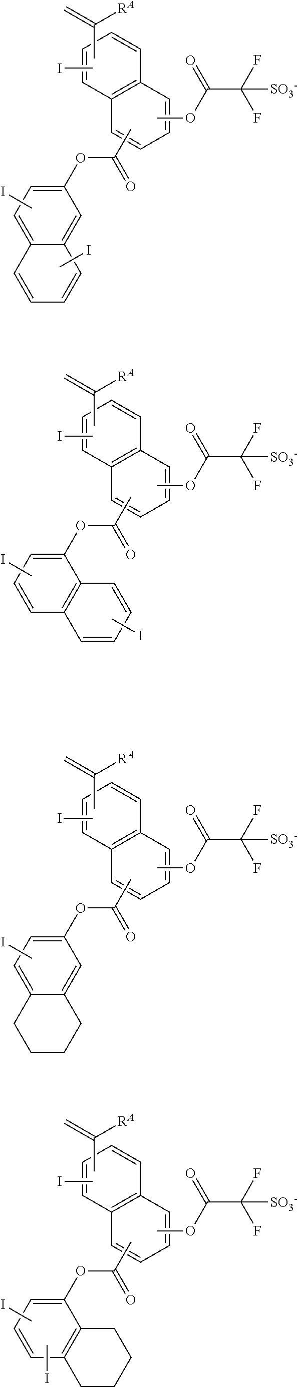

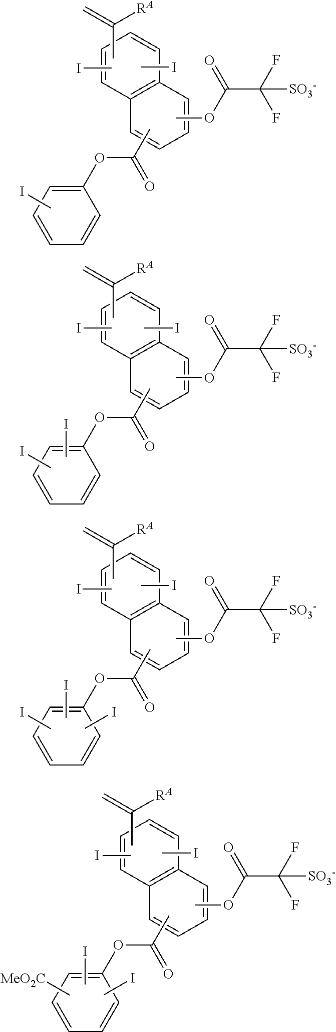

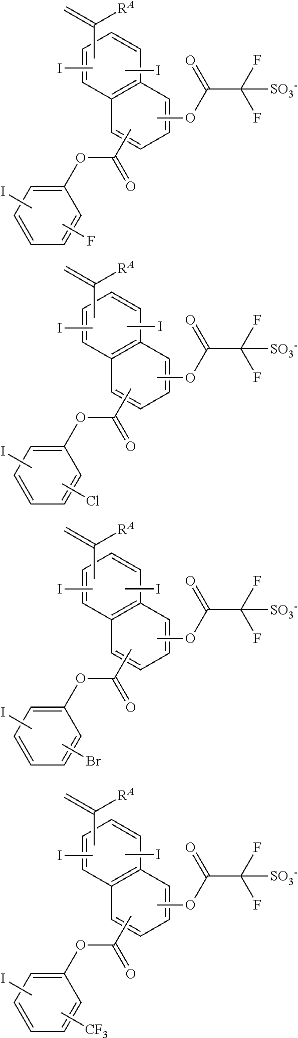

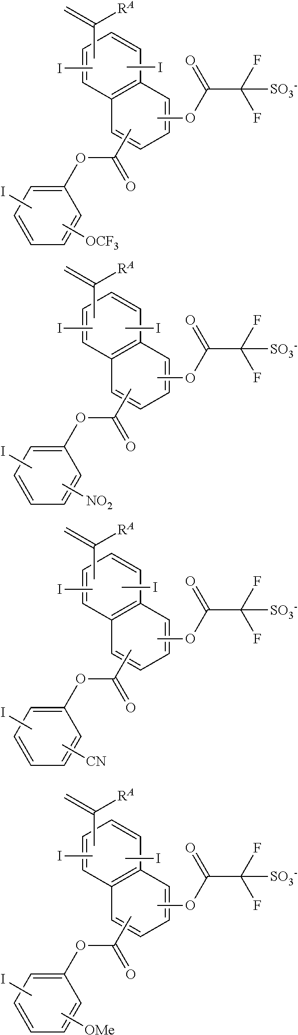

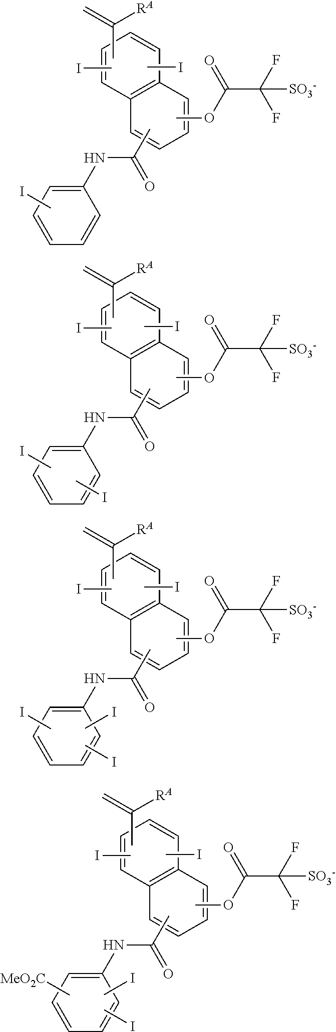

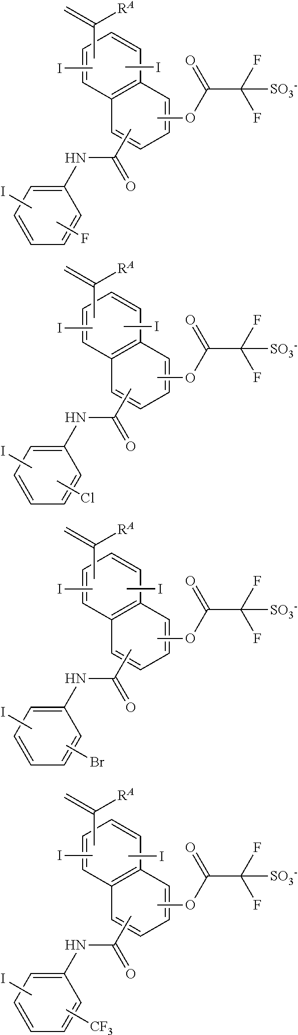

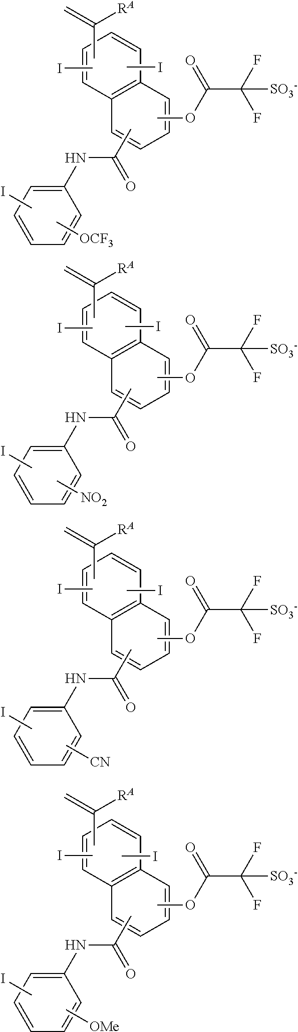

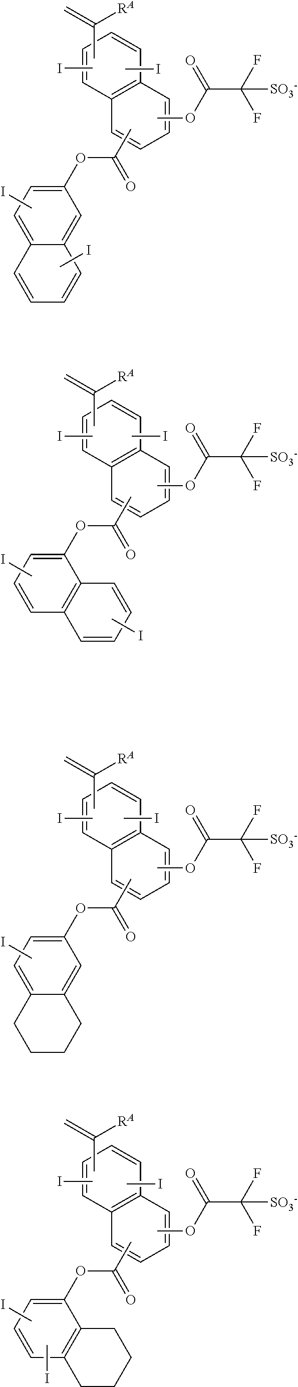

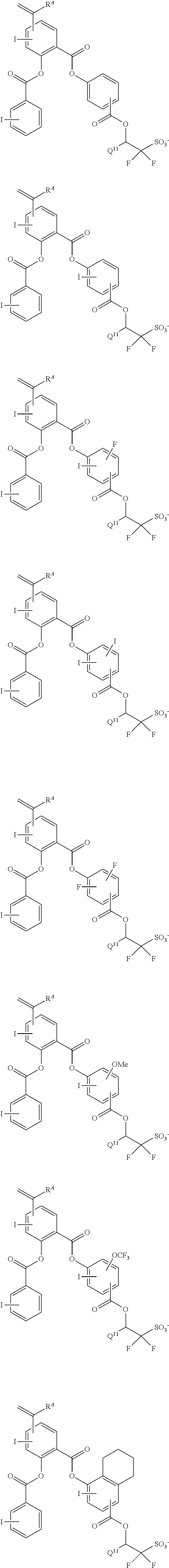

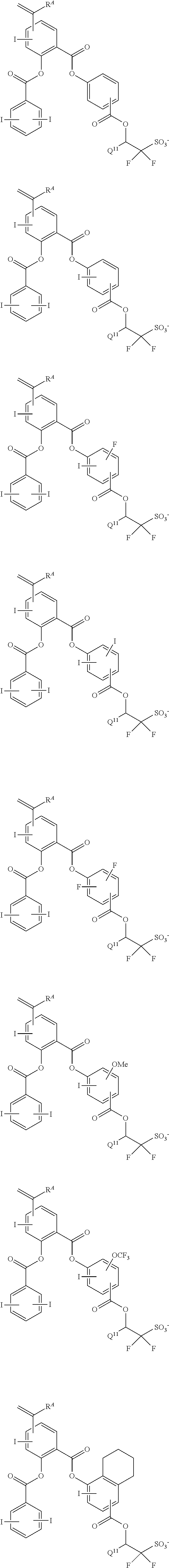

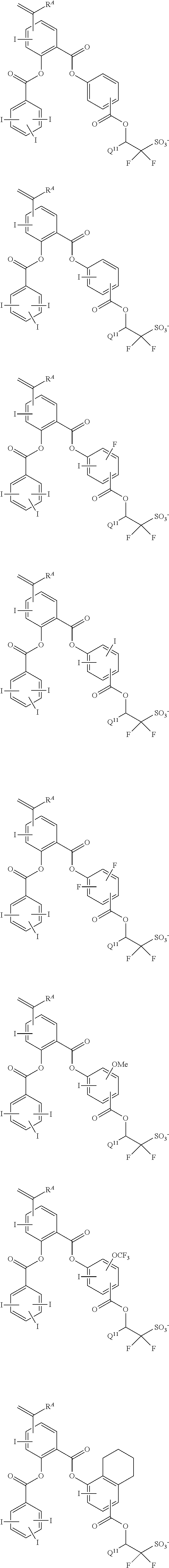

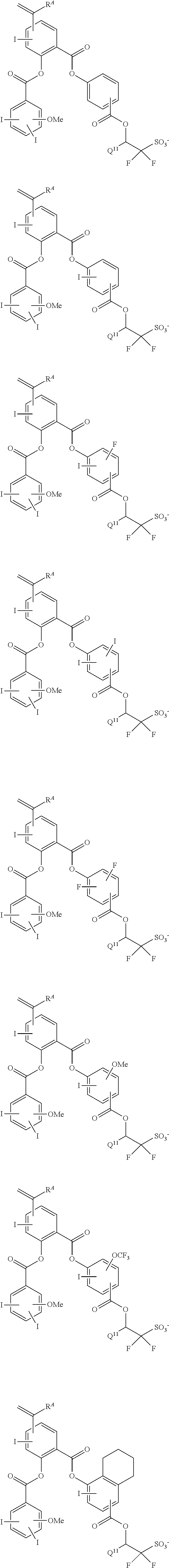

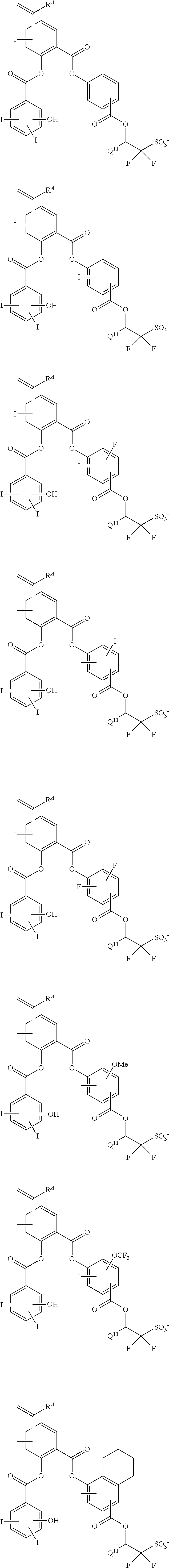

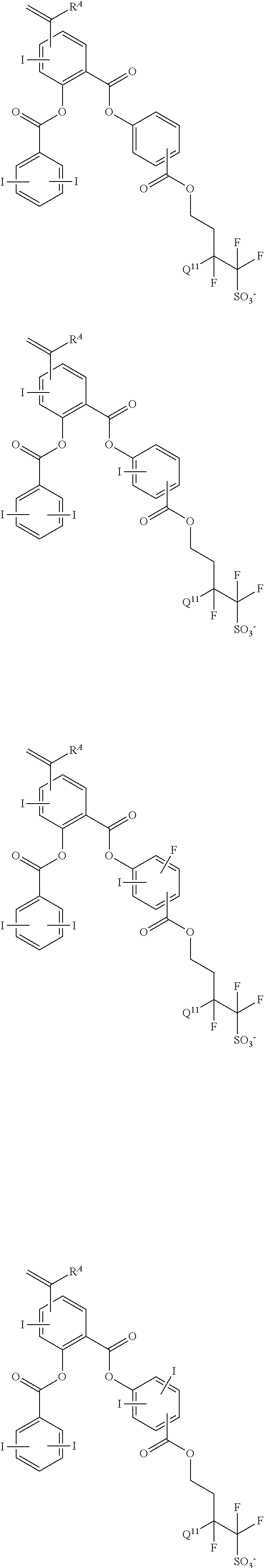

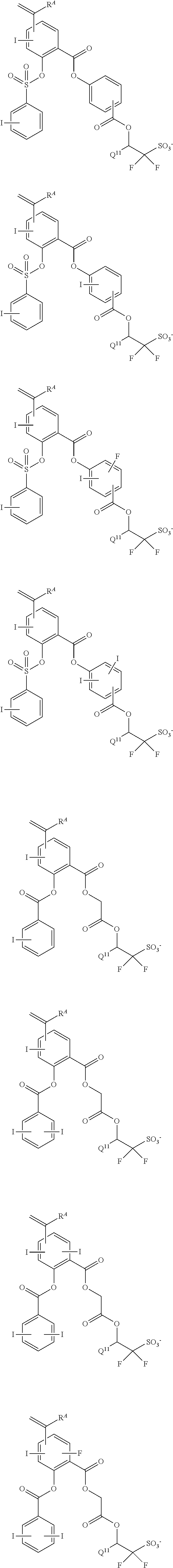

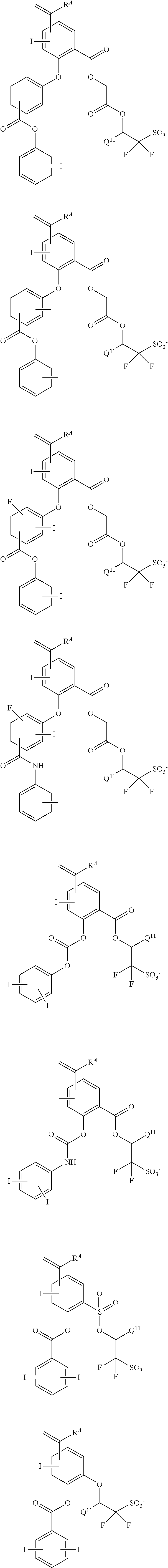

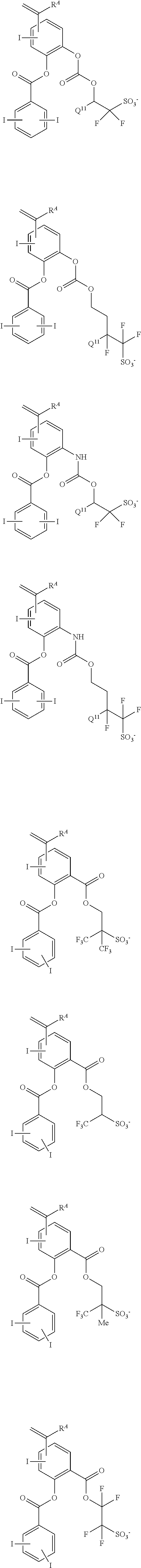

Examples of the sulfonium cation in the repeat units (d) include sulfonium cations in the sulfonium salt monomer of formula (a), and are as described in JP-A 2024-3744, paragraphs [0102]-[0125] and JP-A 2023-169812, paragraphs [0070]-[0085].

As the sulfonium cation in the repeat units (d), structures having the formula (d3) are also preferred.

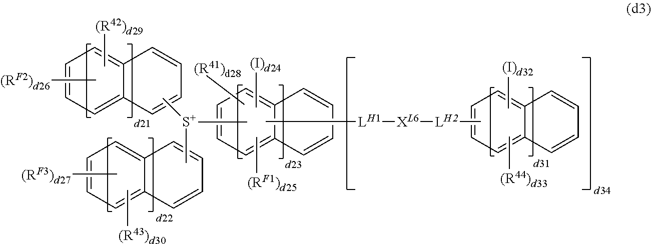

In formula (d3), d21 is 0 or 1. The sulfonium salt monomer has a benzene ring when d21 is 0, and a naphthalene ring when d21 is 1, and d21 is preferably 0 from the aspect of solvent solubility. The subscript d22 is 0 or 1. The sulfonium salt monomer has a benzene ring when d22 is 0, and a naphthalene ring when d22 is 1, and d22 is preferably 0 from the aspect of solvent solubility. The subscript d23 is 0 or 1. The sulfonium salt monomer has a benzene ring when d23 is 0, and a naphthalene ring when d23 is 1, and d23 is preferably 0 from the aspect of solvent solubility.

In formula (d3), d24 is 0, 1, 2, 3 or 4. As the number of iodine atoms in the cationic structure becomes larger, the amount of absorption of EUV increases, but precipitation in the resist composition may occur due to reduced solvent solubility. Therefore, d24 is preferably 0, 1, 2 or 3, more preferably, 0, 1 or 2.

In formula (d3), d25 is 0, 1, 2, 3 or 4. From the aspect of reactant availability, d25 is preferably 0, 1, 2 or 3, more preferably 0, 1 or 2. The subscript d26 is 0, 1, 2, 3, 4, 5 or 6. From the aspect of reactant availability, d26 is preferably 0, 1, 2 or 3, more preferably 0, 1 or 2. The subscript d27 is 0, 1, 2, 3, 4, 5 or 6. From the aspect of reactant availability, d27 is preferably 0, 1, 2 or 3, more preferably 0, 1 or 2.

In formula (d3), d28 is 0, 1 or 2. From the aspect of reactant availability, d28 is preferably 0 or 1. The subscript d29 is 0, 1 or 2. From the aspect of reactant availability, d29 is preferably 0 or 1. The subscript d30 is 0, 1 or 2. From the aspect of reactant availability, d30 is preferably 0 or 1.

In formula (d3), d31 is 0 or 1. The sulfonium salt monomer has a benzene ring when d31 is 0, and a naphthalene ring when d31 is 1, and d31 is preferably 0 from the aspect of solvent solubility.

In formula (d3), d32 is 0, 1, 2, 3 or 4. As the number of iodine atoms in the cationic structure becomes larger, the amount of absorption of EUV increases, but precipitation in the resist composition may occur due to reduced solvent solubility. Therefore, d32 is preferably 0, 1, 2 or 3, more preferably, 0, 1 or 2.

In formula (d3), d33 is 0, 1 or 2. From the aspect of reactant availability, d33 is preferably 0 or 1. The subscript d34 is 0, 1 or 2. From the aspect of synthesis, d34 is preferably 0 or 1.

The sum of d26+d29 is from 0 to 4 when d21 is 0, and the sum of d26+d29 is from 0 to 6 when d21 is 1. The sum of d27+d30 is from 0 to 4 when d22 is 0, and the sum of d27+d30 is from 0 to 6 when d22 is 1. The sum of d24+d25+d28+d34 is from 1 to 4 when d23 is 0, and the sum of d24+d25+d28+d4 is from 1 to 6 when d23 is 1. The sum of d32+d33 is from 0 to 4 when d31 is 0, and the sum of d32+d33 is from 0 to 6 when d31 is 1. The sum of d24+d32 is 1 or less.

In formula (d3), RF¹ to R^{F3} are each independently fluorine, a C₁-C₆ fluorinated saturated hydrocarbyl group, a C₁-C₆ fluorinated saturated hydrocarbyloxy group, or a C₁-C₆ fluorinated saturated hydrocarbylthio group. Of these, trifluoromethyl group, trifluoromethoxy group and trifluorothiomethoxy group are preferred. A plurality of R^{F1} may be identical or different when d25 is 2 or more, a plurality of R^{F2} may be identical or different when d26 is 2 or more, and plurality of R^{F3} may be identical or different when d27 is 2 or more.

In formula (d3), each of R⁴¹ to R⁴⁴ is halogen exclusive of iodine and fluorine, nitro group, cyano group, a C₁-C₂₀ hydrocarbyl group which may contain a heteroatom, a C₁-C₂₀ hydrocarbyloxy group which may contain a heteroatom, a C₁-C₂₀ hydrocarbyloxy group, or a C₁-C₂₀ hydrocarbylthio group which may contain a heteroatom. The hydrocarbyl group and hydrocarbyl moiety of the hydrocarbyloxy and hydrocarbylthio groups may be saturated or unsaturated and straight, branched or cyclic. Examples of thereof are as exemplified above as the hydrocarbyl group R¹ in formula (1). In the hydrocarbyl group and the hydrocarbyl moieties of the hydrocarbyloxy group and hydrocarbylthio group, some or all hydrogen atoms may be substituted by a moiety containing a heteroatom such as oxygen, sulfur, nitrogen or halogen and some constituent —CH₂— may be replaced by a moiety containing a heteroatom such as oxygen, sulfur or nitrogen, so that the group may contain hydroxy, fluorine, chlorine, bromine, iodine, cyano, nitro, mercapto, pentafluorosulfanyl, carbonyl, ether bond, ester bond, sulfonic ester bond, carbonate bond, lactone ring, sultone ring, carboxylic anhydride (—C(═O)—O—C(═O)—), or haloalkyl moiety.

Two R⁴¹ may be identical or different and the two R⁴¹ may bond together to form a ring with the carbon atoms to which they are attached when d28 is 2, two R⁴² may be identical or different and the two R⁴² may bond together to form a ring with the carbon atoms to which they are attached when d29 is 2, two R⁴³ may be identical or different and the two R⁴³ may bond together to form a ring with the carbon atoms to which they are attached when d30 is 2, and two R⁴⁴ may be identical or different and the two R⁴⁴ may bond together to form a ring with the carbon atoms to which they are attached when d33 is 2. Examples of the ring formed herein include cyclopropane, cyclobutane, cyclopentane, cyclohexane, norbornane and adamantane rings. Some or all of hydrogen atoms in the ring may be replaced by a group containing a heteroatom such as oxygen, sulfur, nitrogen or halogen, some constituent —CH₂— in the ring may be replaced by a group containing a heteroatom such as oxygen, sulfur or nitrogen, and as a result, the hydrocarbyl group may contain a hydroxy group, fluorine, chlorine, bromine, iodine, a cyano group, a carbonyl group, an ether bond, an ester bond, a sulfonic ester bond, a carbonate bond, a lactone ring, a sultone ring, carboxylic anhydride (—C(═O)—O—C(═O)—), a haloalkyl group, or the like.

The aromatic rings directly bonded to S⁺ in the sulfonium cation of formula (d3) may bond together to form a ring with S⁺. Examples of the structure of the ring include those represented by the following formula.

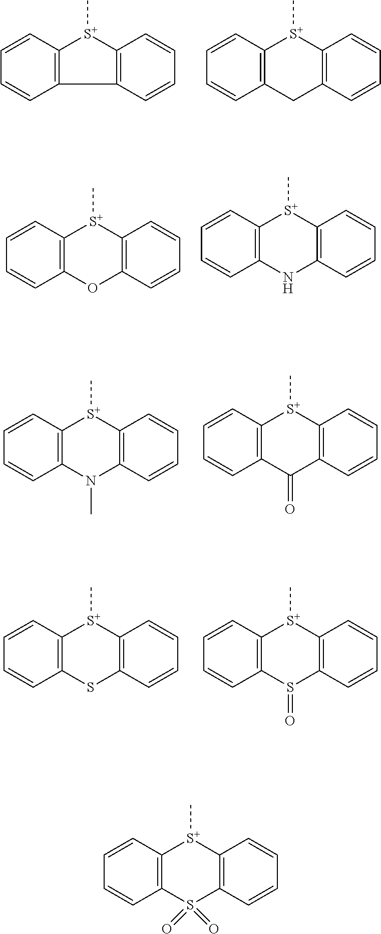

Herein the broken line denotes a point of attachment.

In formula (d3), L^{H1} and L^{H2} are each independently a single bond, ether bond, ester bond, amide bond, sulfonic ester bond, sulfonic amide bond, carbonate bond or carbamate bond. Inter alia, L^{H1} is preferably a single bond, ether bond, ester bond or sulfonic ester bond, more preferably ester bond or sulfonic ester bond. L^{H2} is preferably a single bond, an ether bond or an ester bond, more preferably a single bond.

In formula (d3), X^{L6} is a single bond or C₁-C₄₀ hydrocarbylene group which may contain a heteroatom. The hydrocarbylene group may be straight, branched or cyclic, and examples thereof include alkanediyl, cyclic saturated hydrocarbylene, and arylene groups. Examples of the heteroatom include oxygen, nitrogen and sulfur atoms. Examples of the C₁-C₄₀ hydrocarbylene group X^{L6} which may contain a heteroatom include X^{L}-0 to X^{L}-58 exemplified for formula (Z) as examples of the C₁-C₄₀ hydrocarbylene groups X^{L1} and X^{L2} which may contain a heteroatom. Of these, X^{L}-0 to X^{L}-22, X^{L}-29 to X^{L}-34, and X^{L}-47 to X^{L}-58 are preferred.

Of the sulfonium cations of formula (d3), a structure having the formula (d3-1) is preferred.

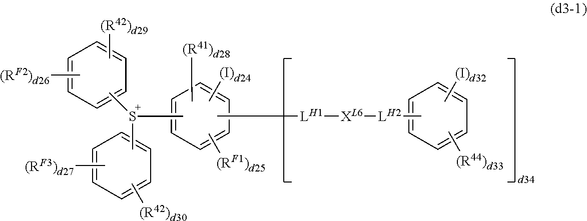

Herein, d24 to d30, d32 to d34, R^{F1} to R^{F3}, R⁴¹ to R⁴⁴, L^{H1}, L^{H2} and X^{L6} are as defined above.

Of the sulfonium cations of formula (d3-1), a structure having the formula (d3-2) is preferred.

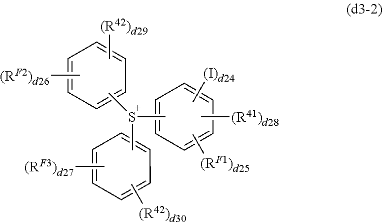

Herein, d24 to d30, R^{F1} to R^{F3} and R^{ct6} to R^{ct8} are as defined above.

Examples of the sulfonium cation of formula (3) are shown below, but not limited thereto. In the following formula, Me is a methyl group.

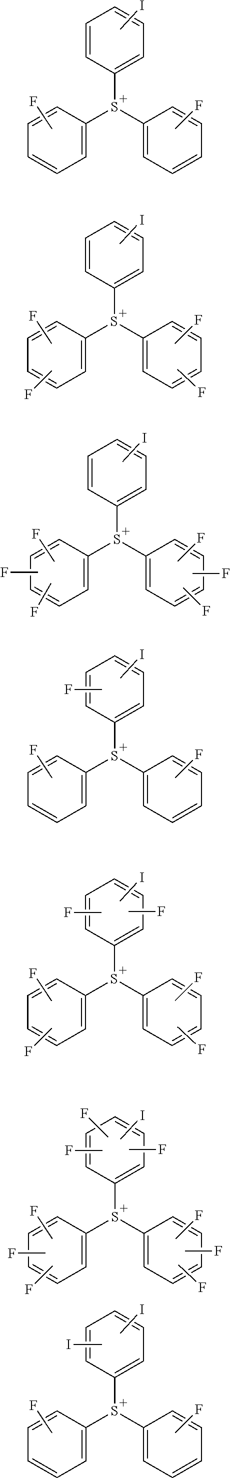

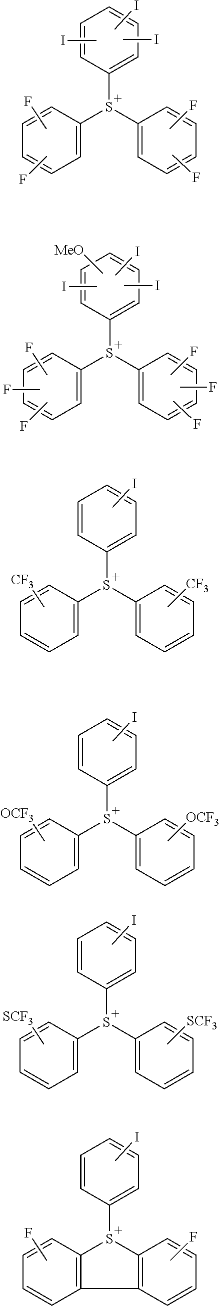

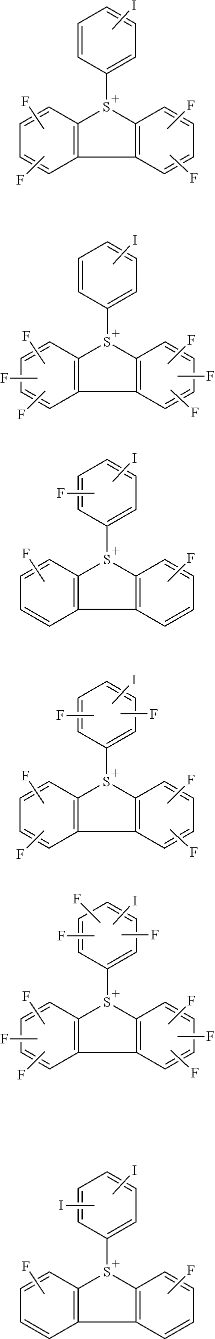

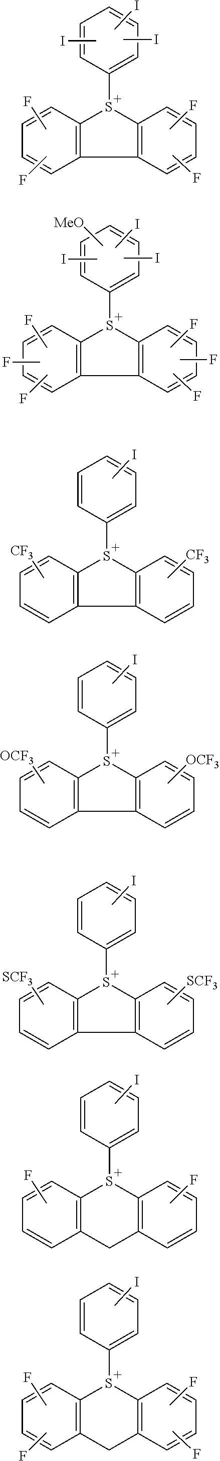

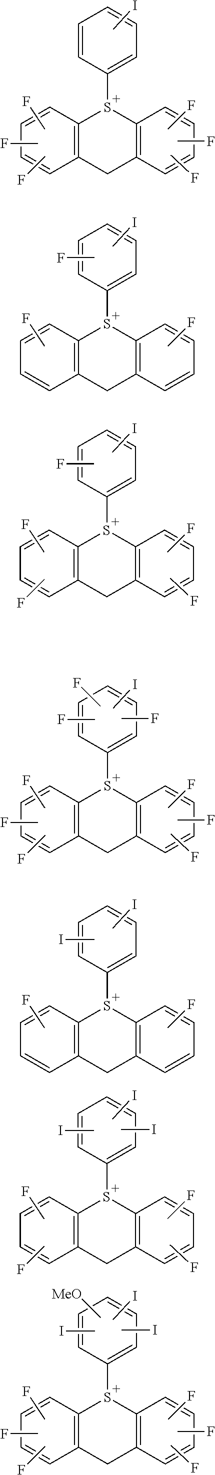

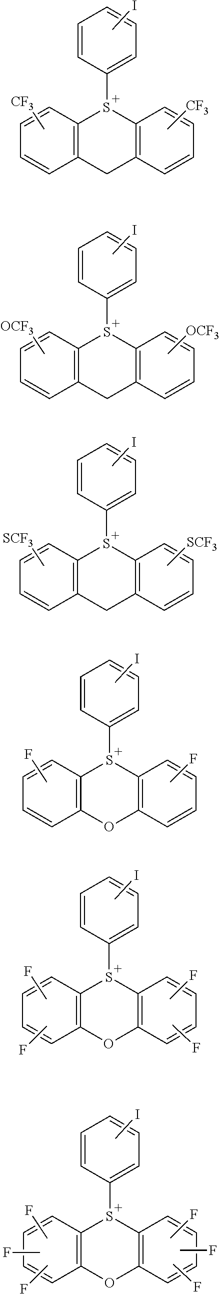

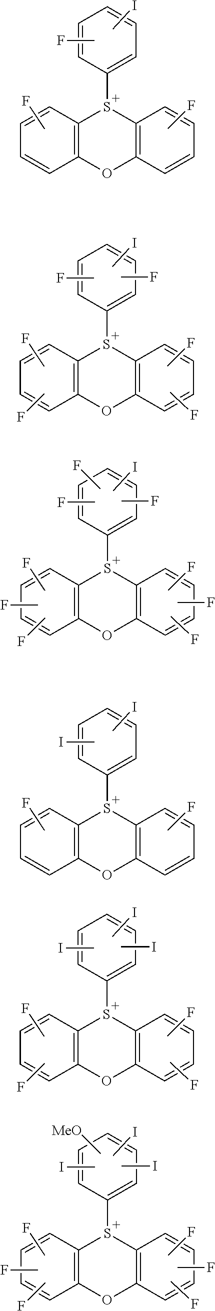

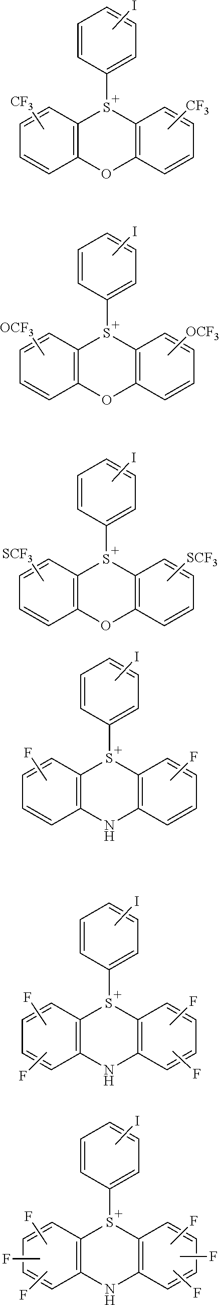

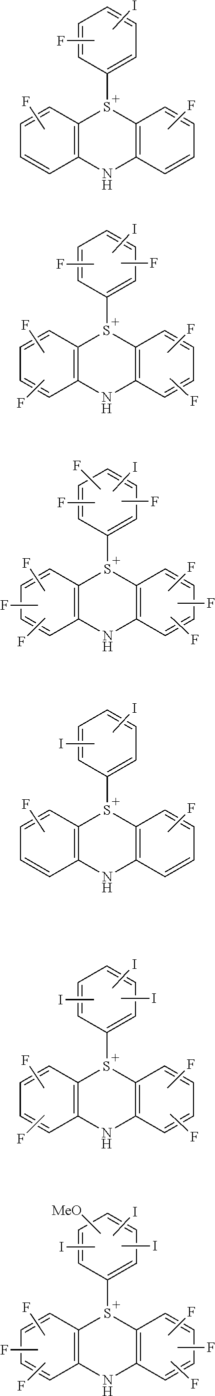

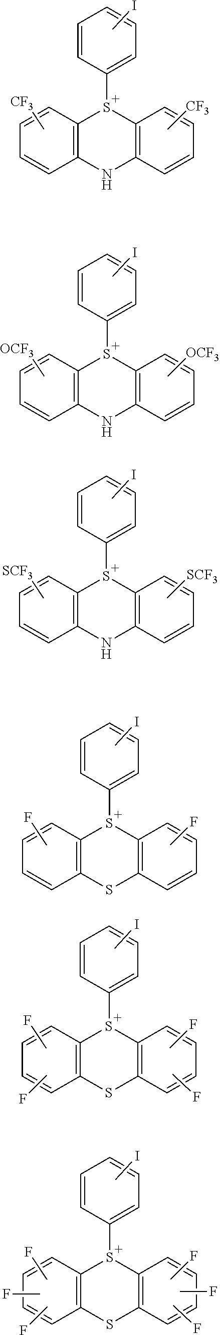

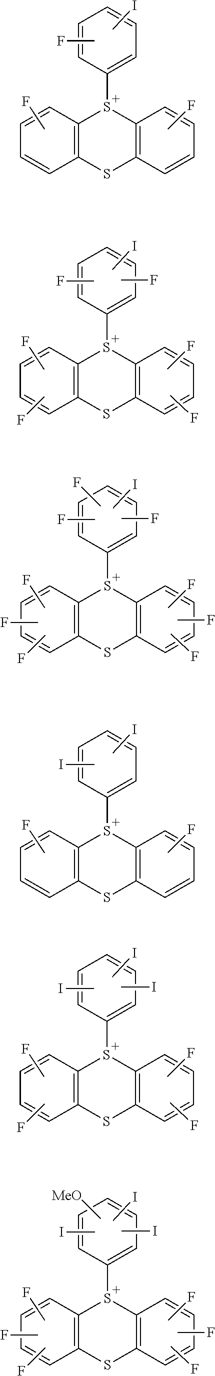

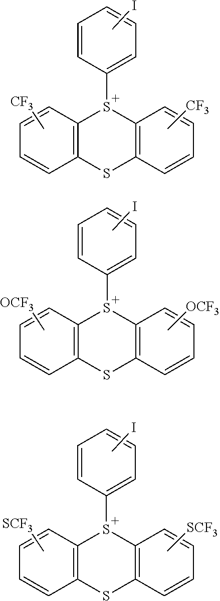

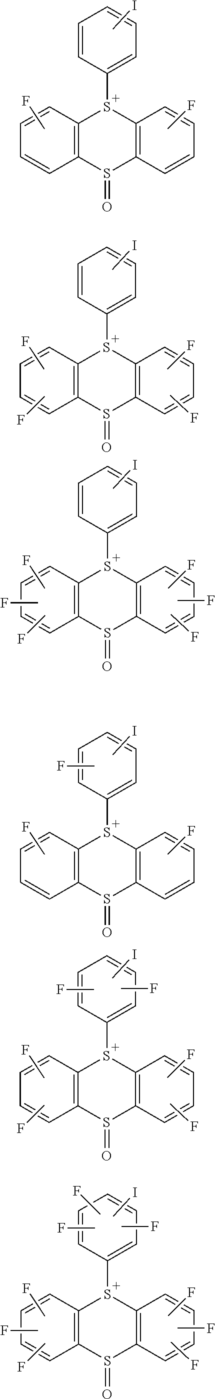

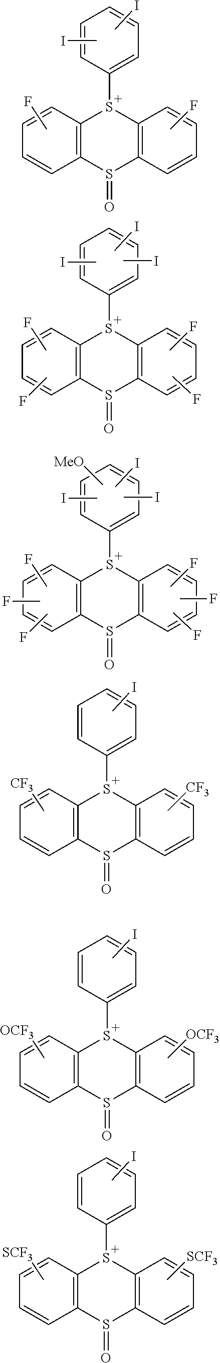

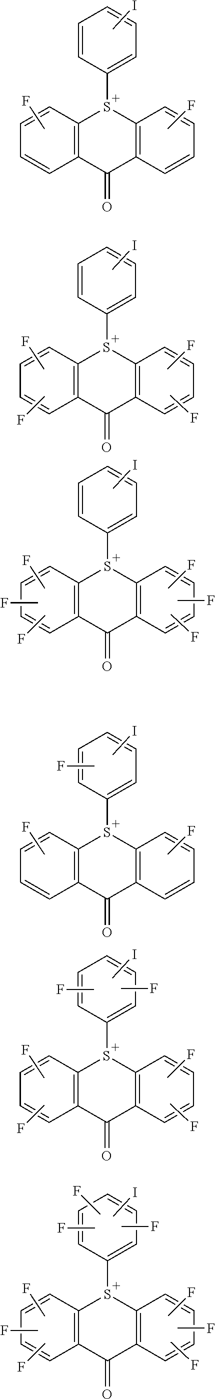

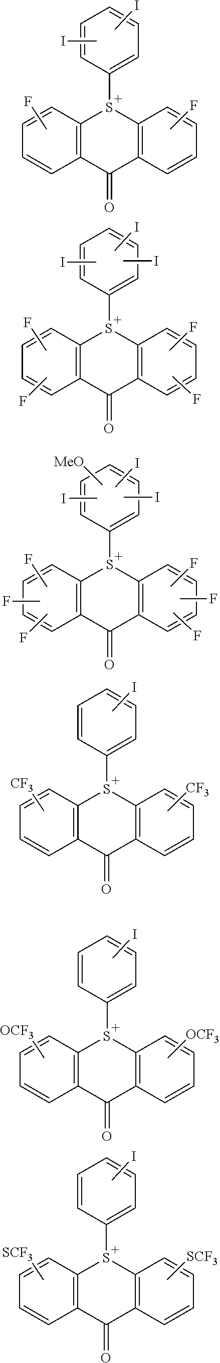

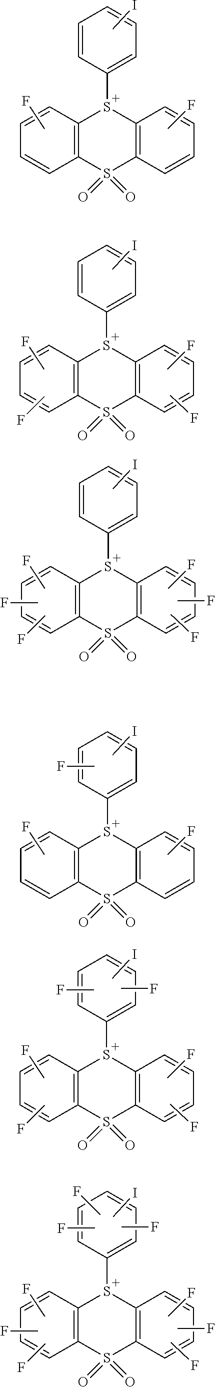

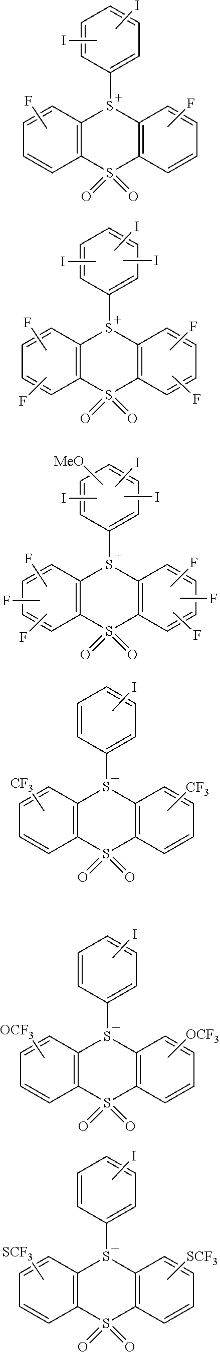

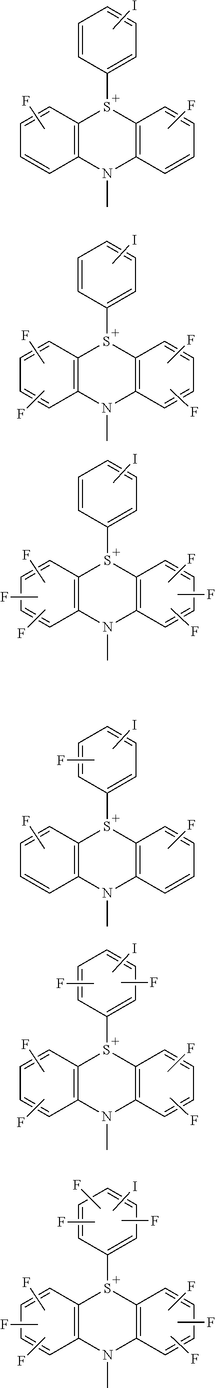

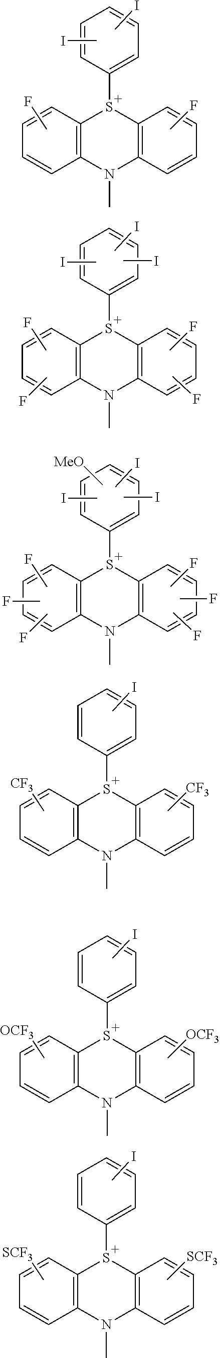

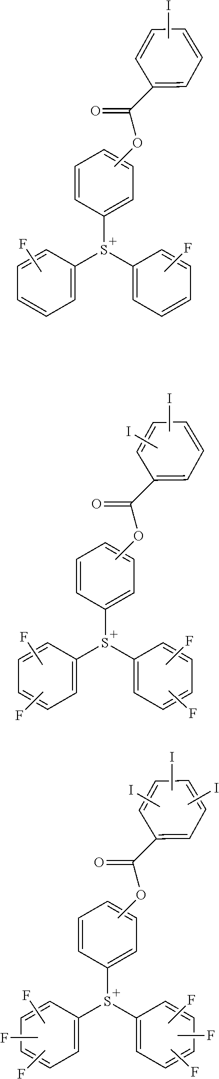

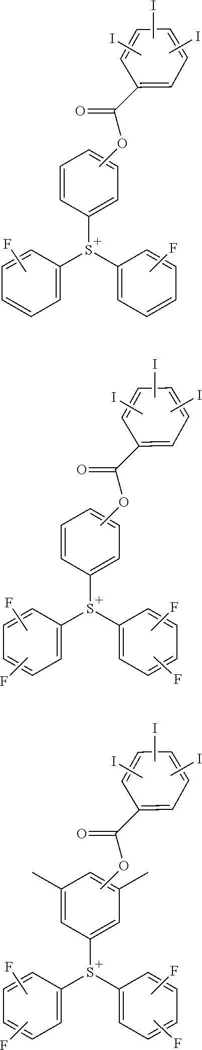

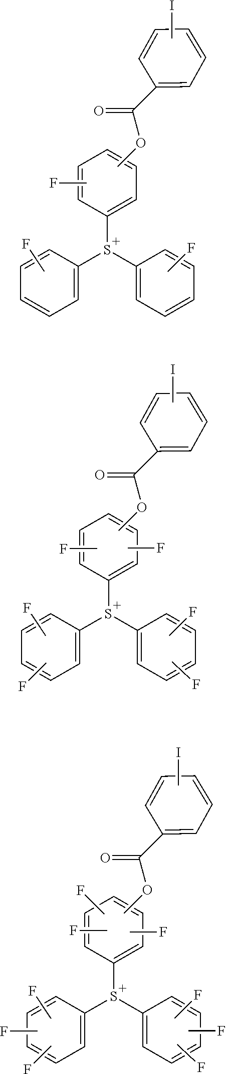

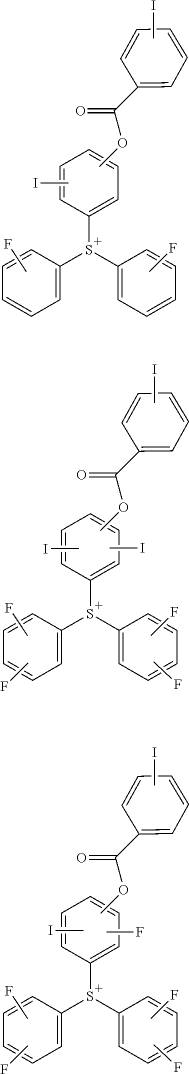

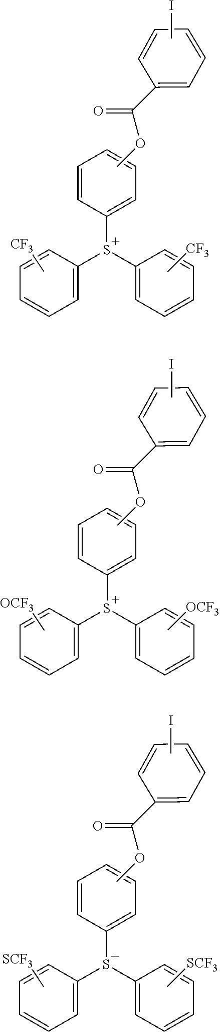

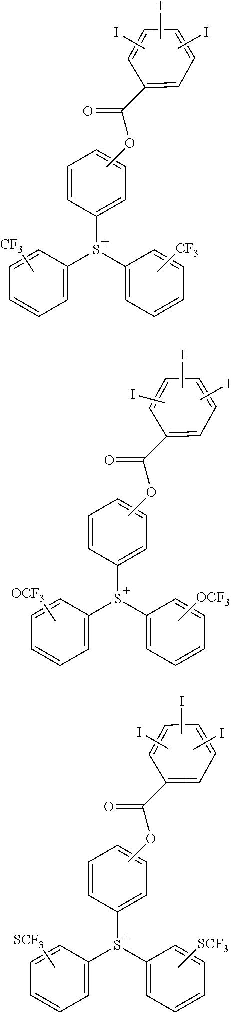

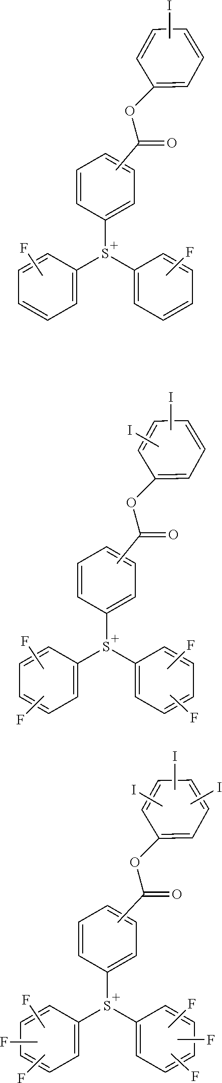

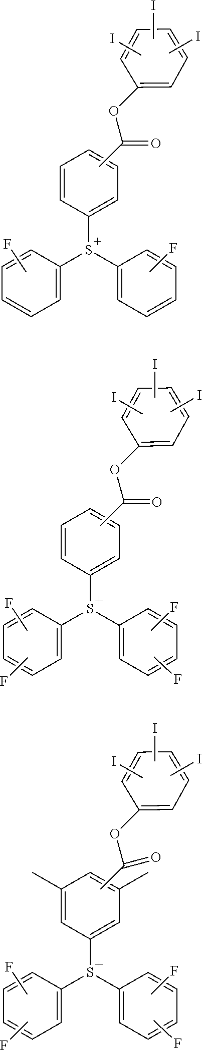

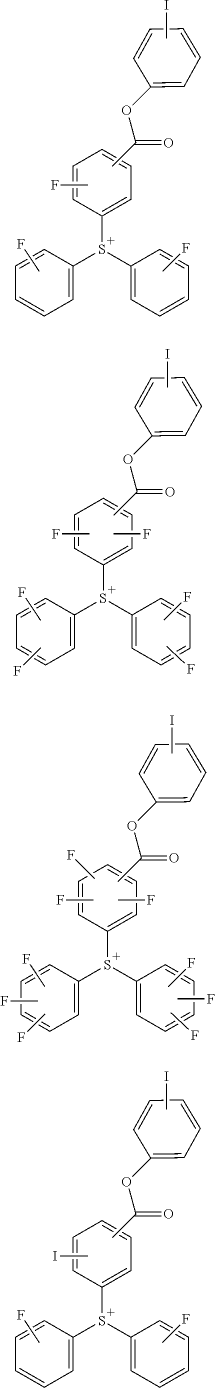

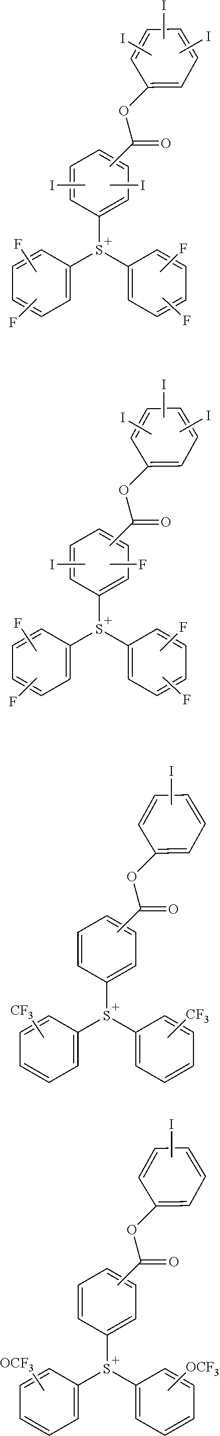

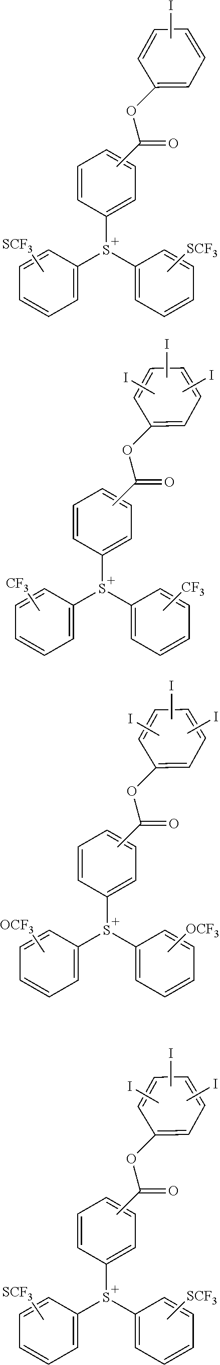

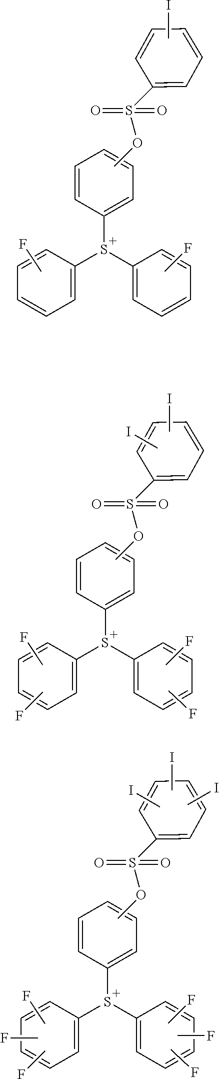

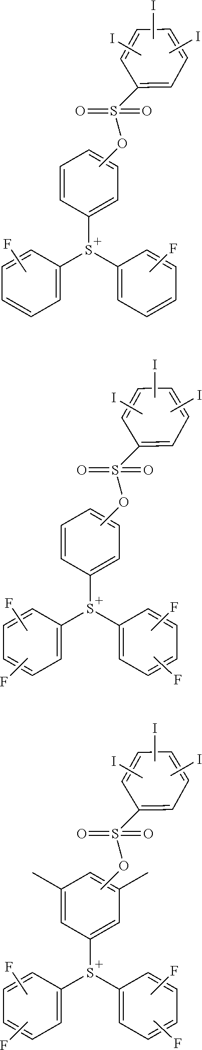

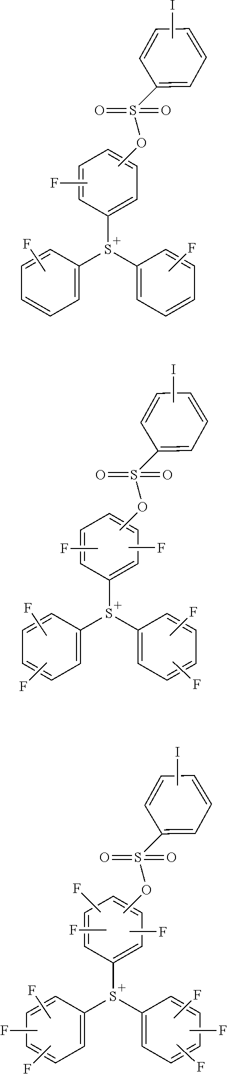

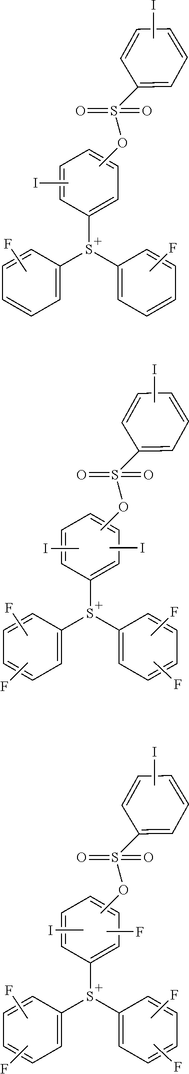

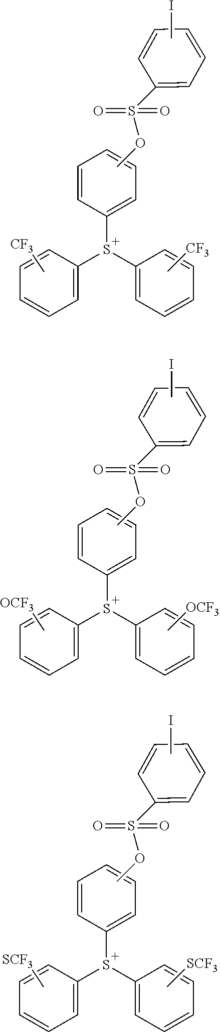

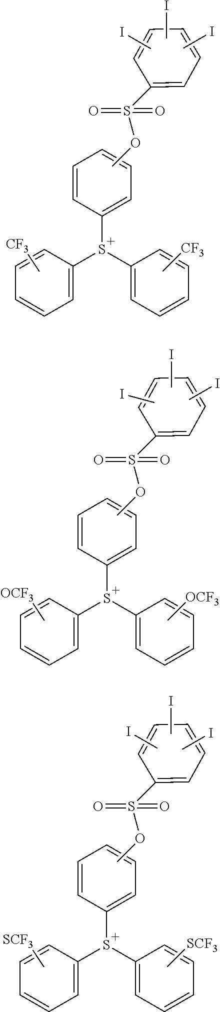

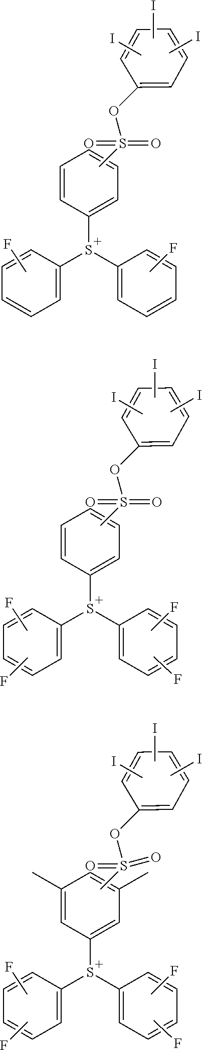

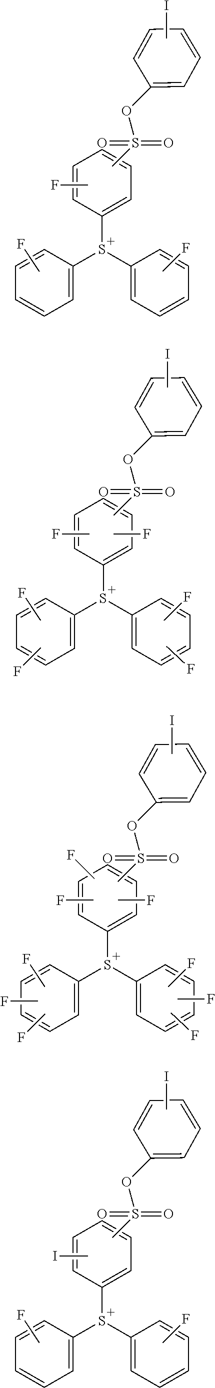

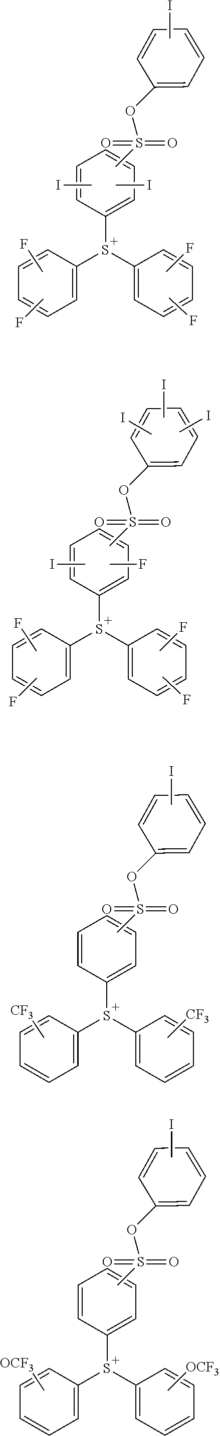

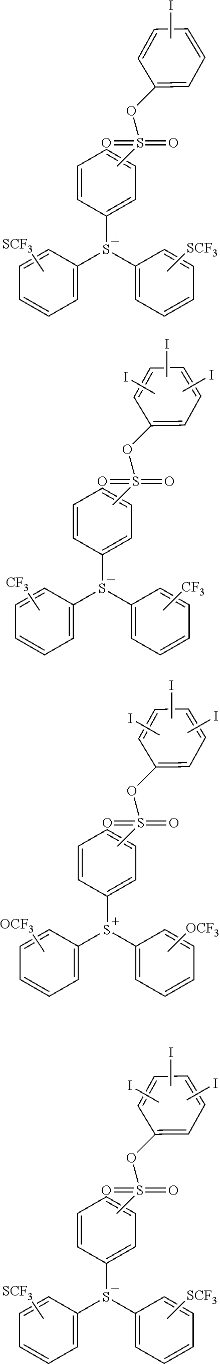

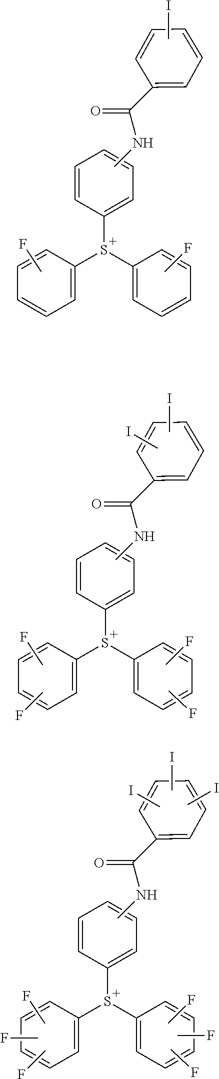

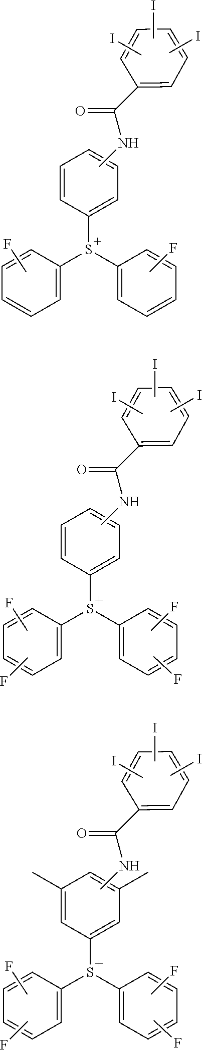

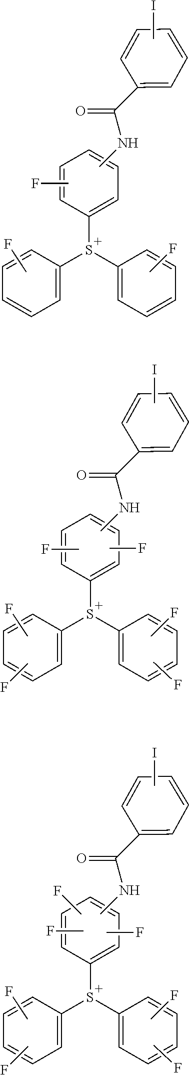

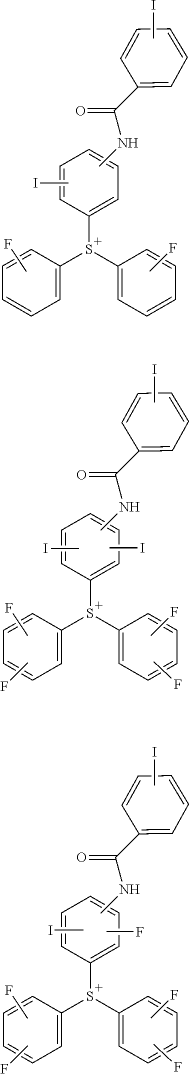

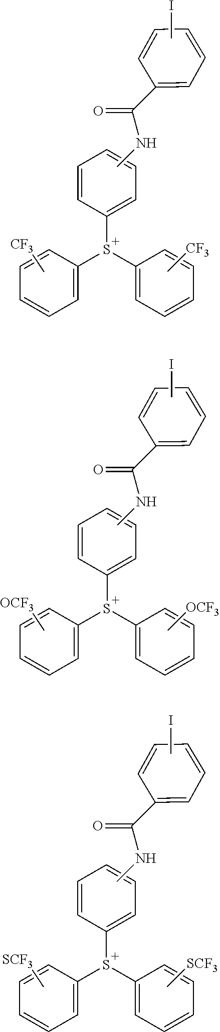

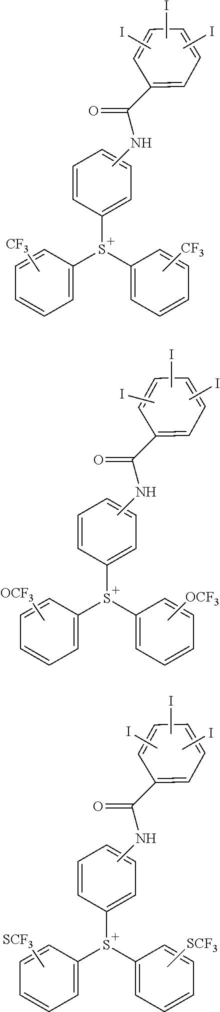

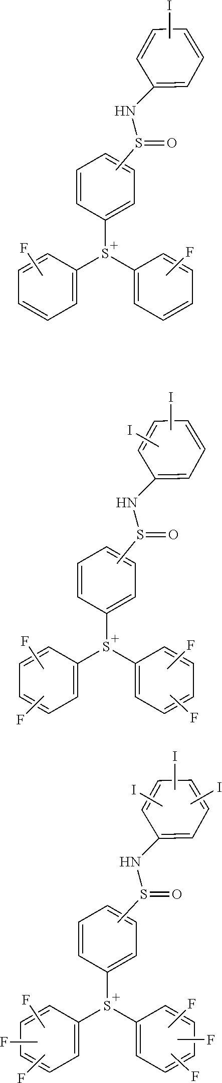

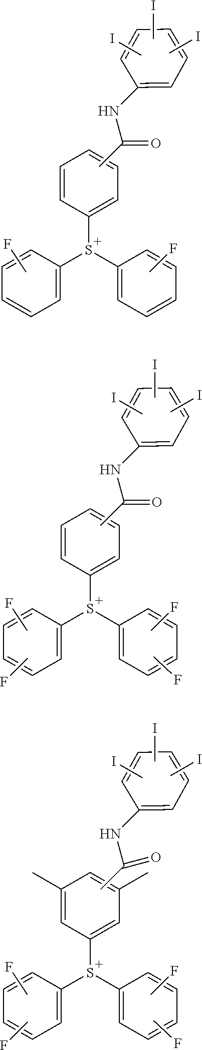

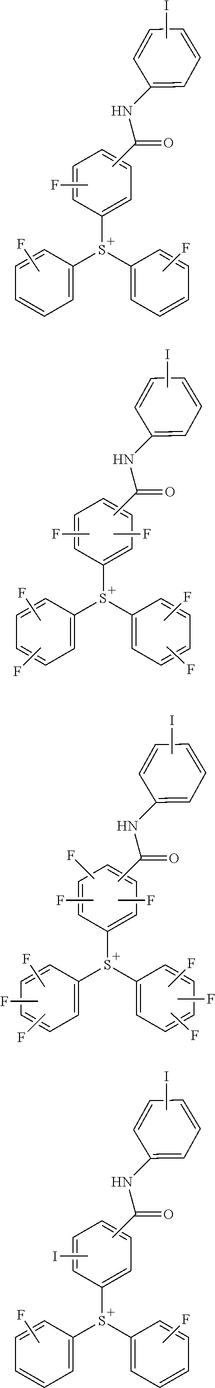

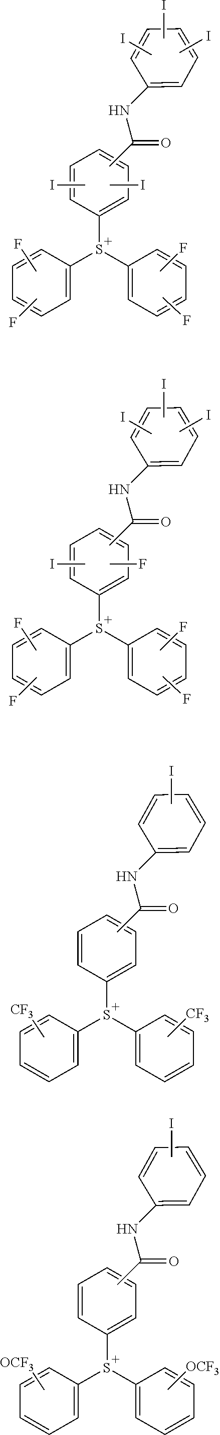

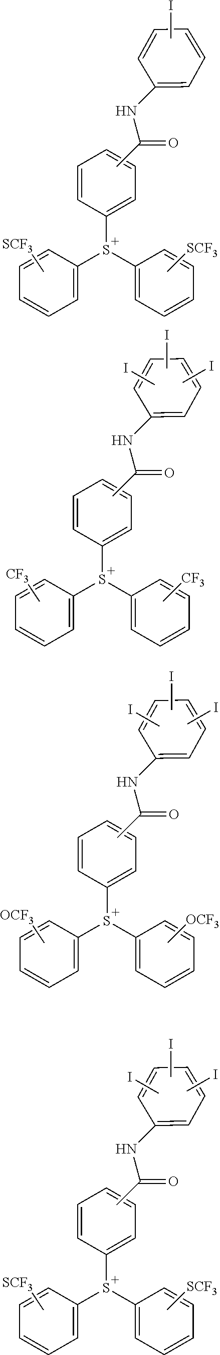

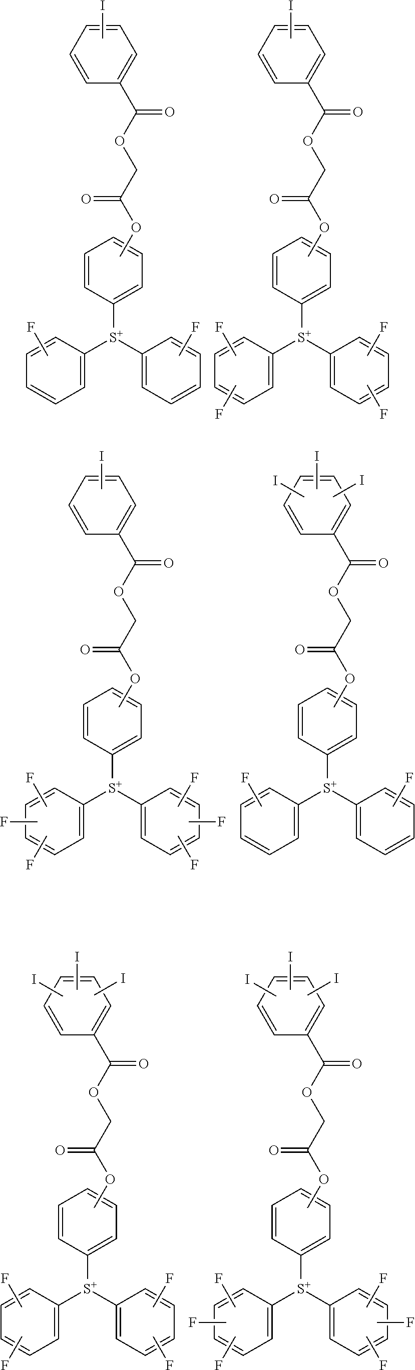

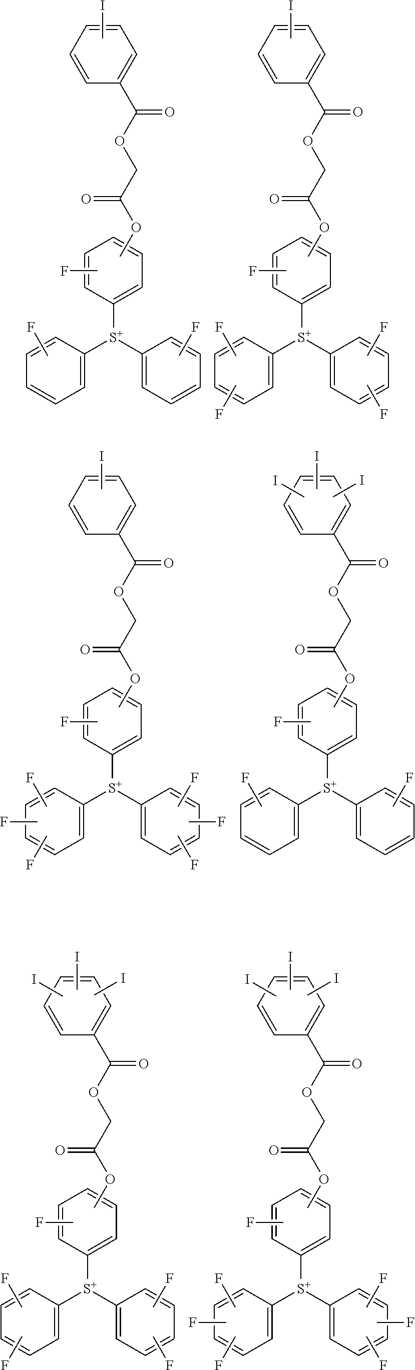

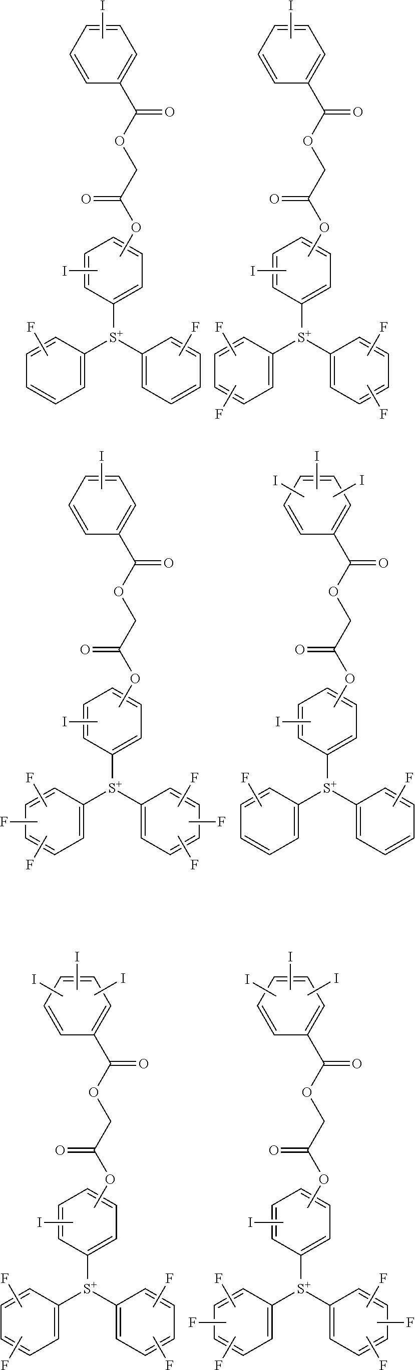

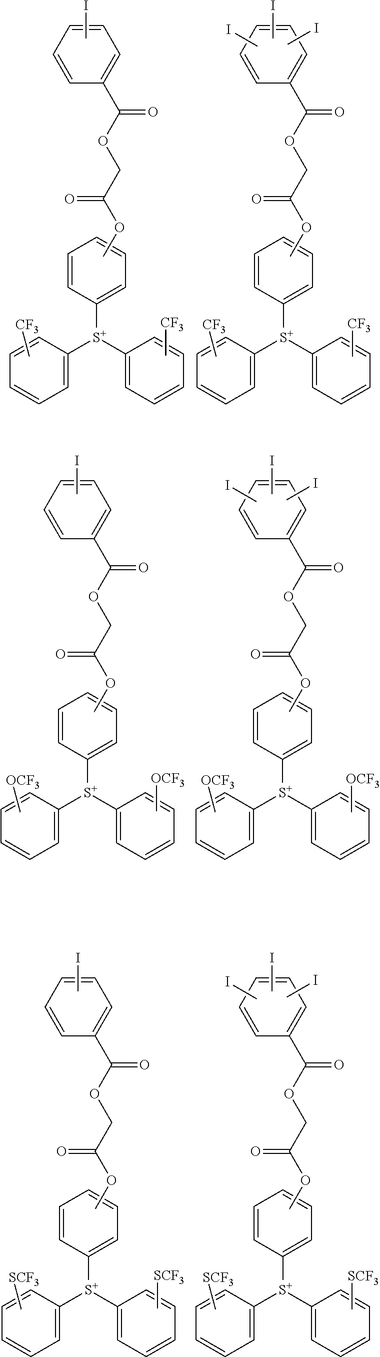

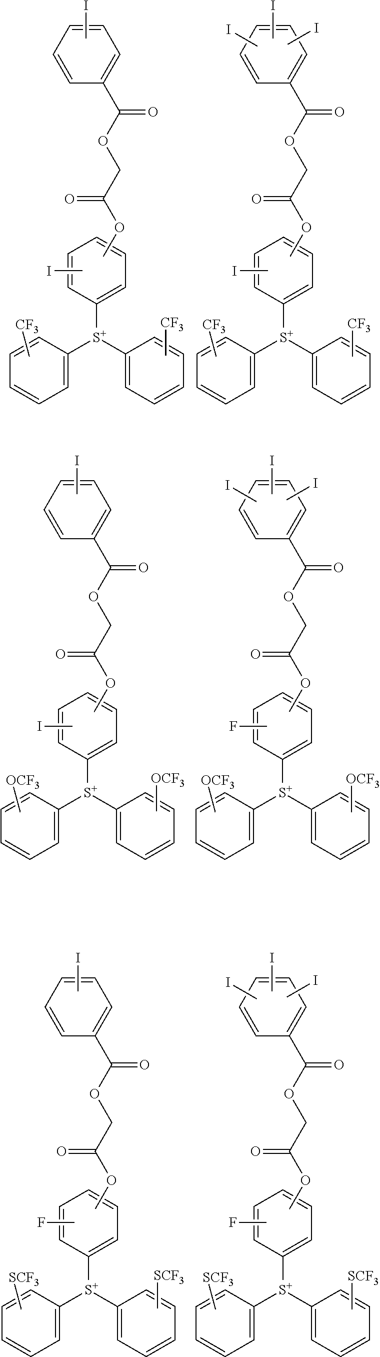

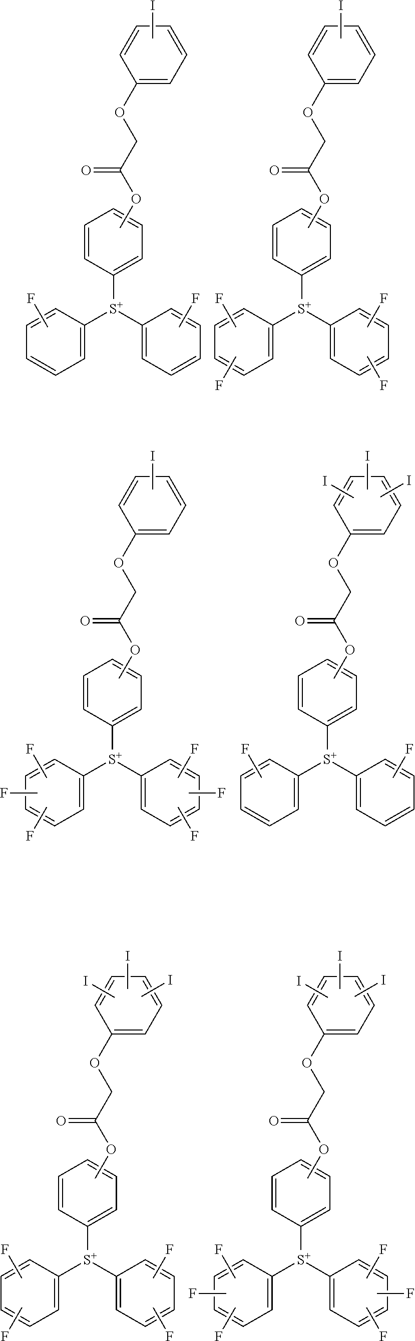

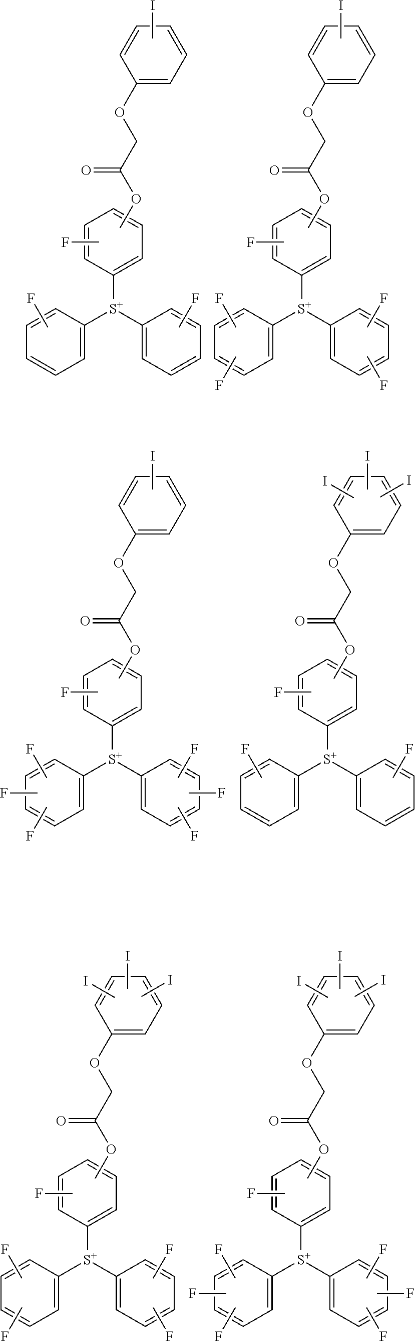

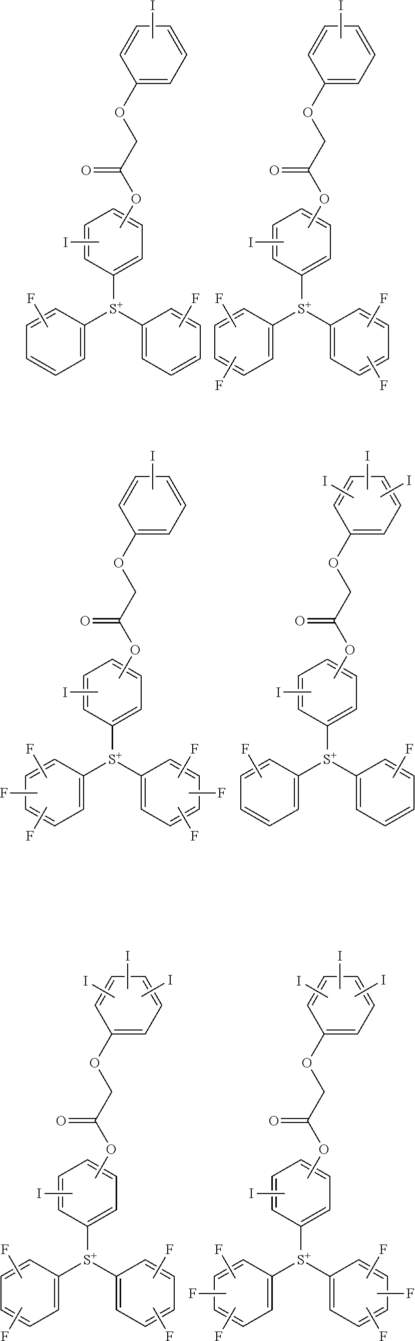

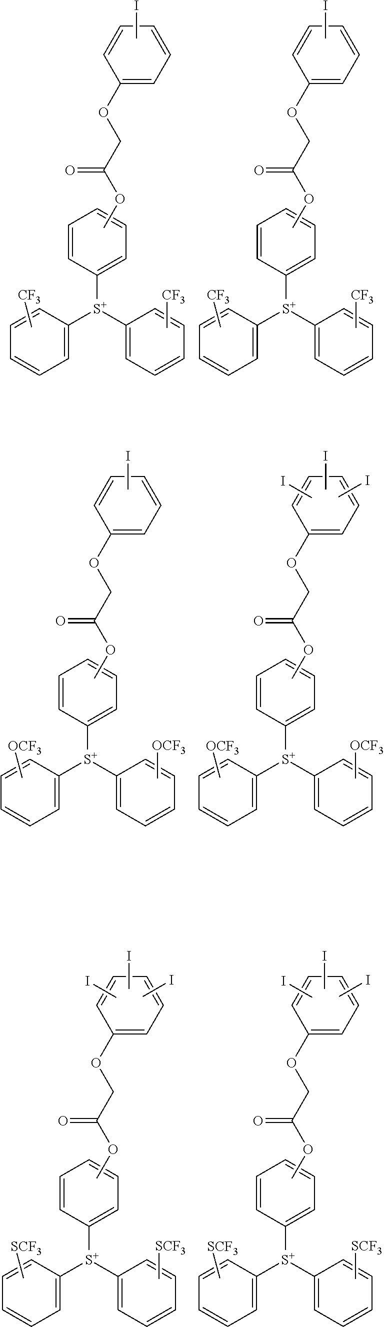

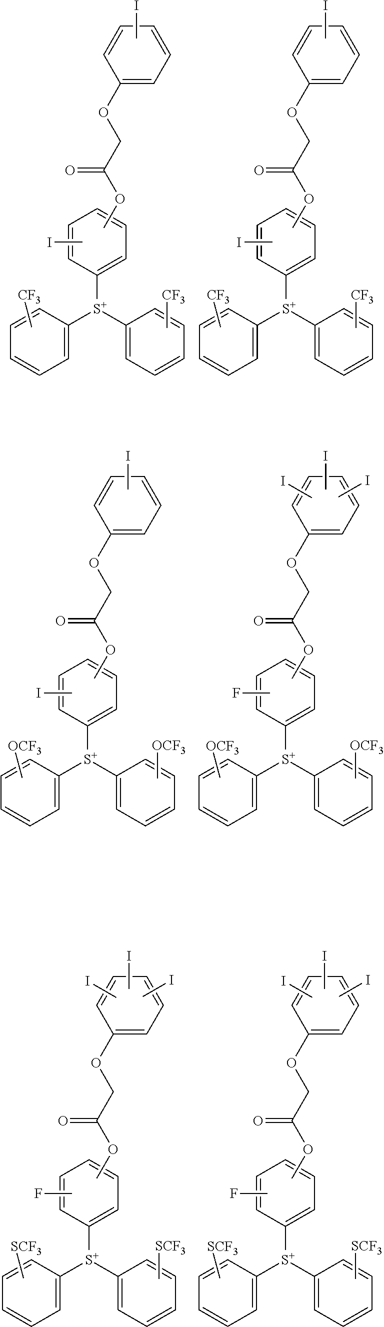

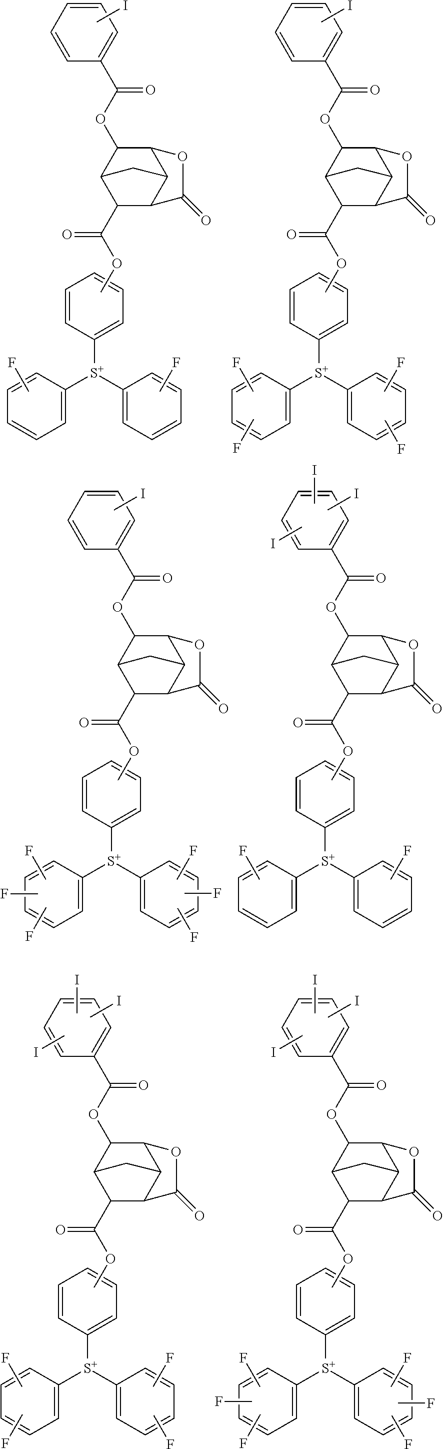

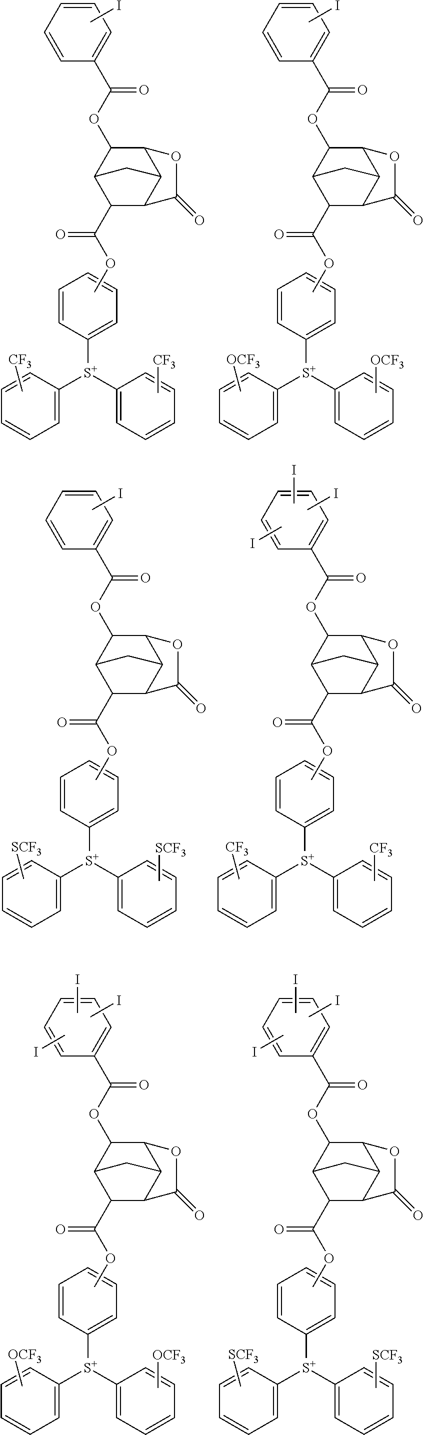

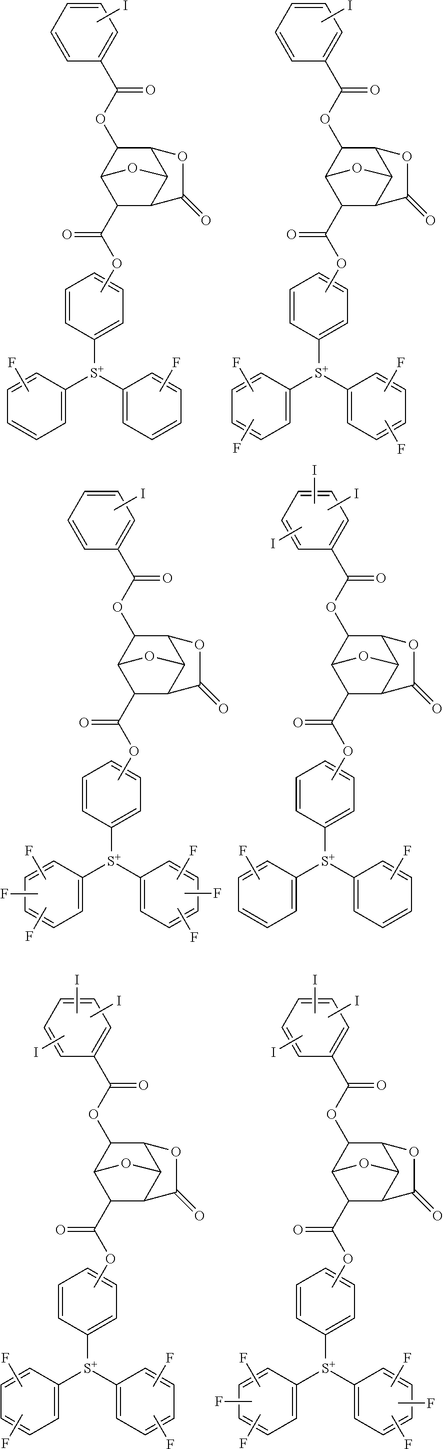

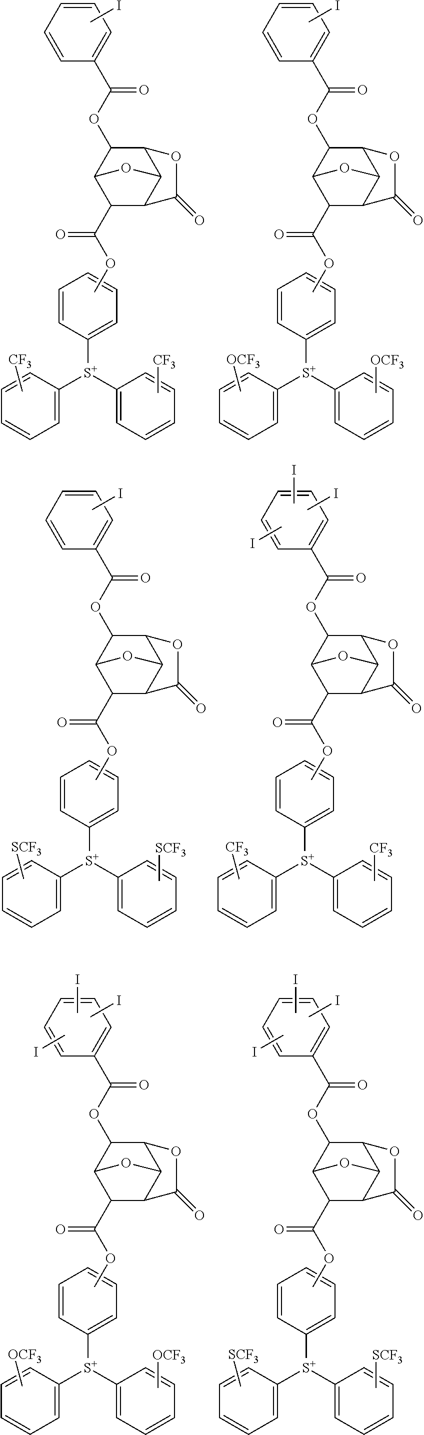

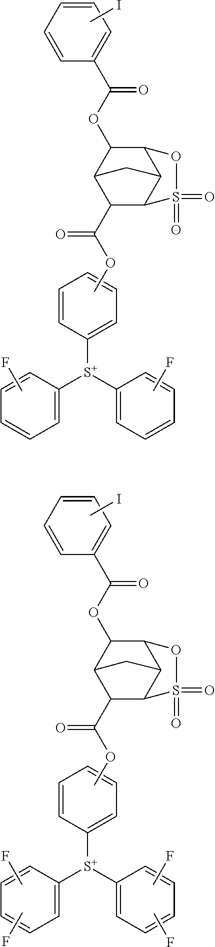

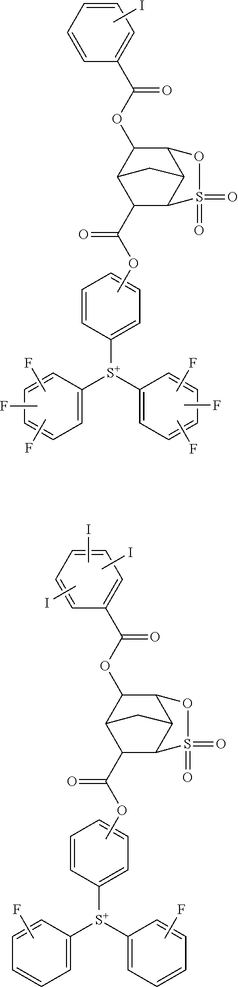

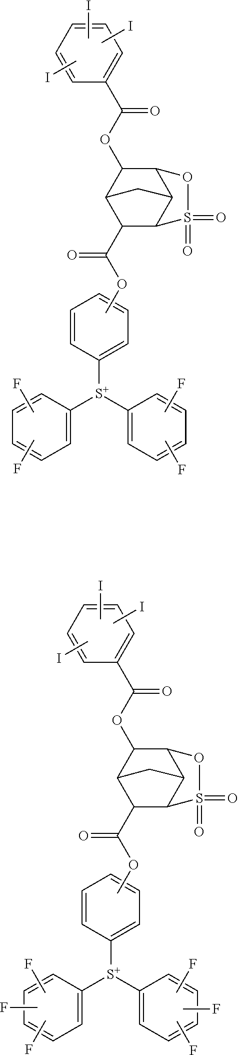

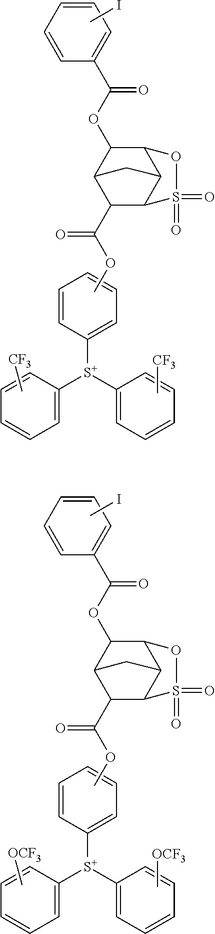

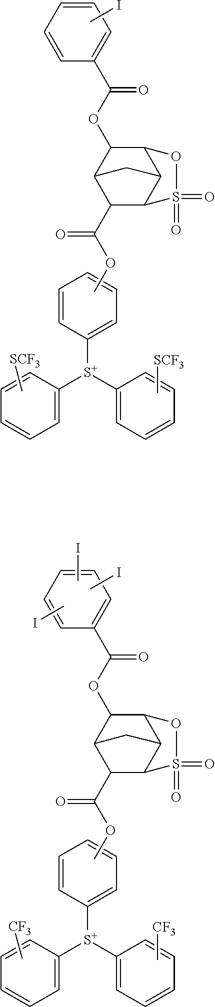

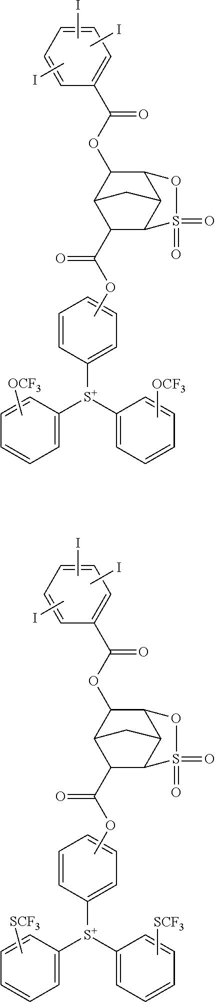

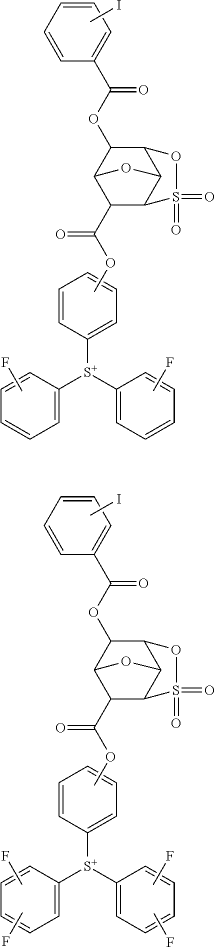

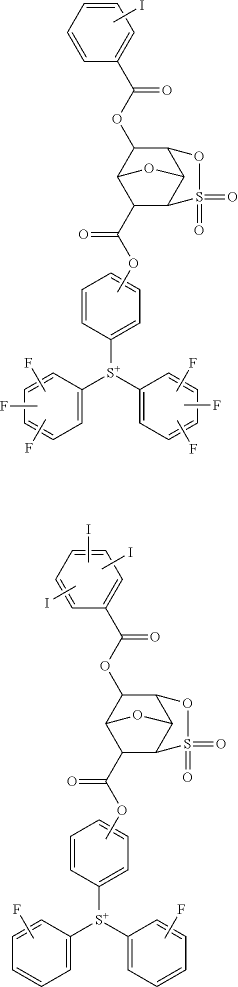

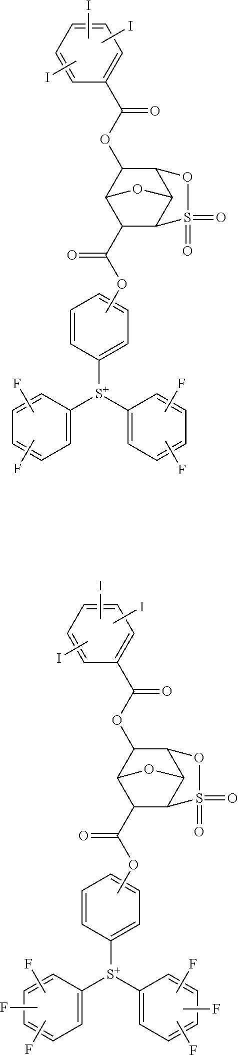

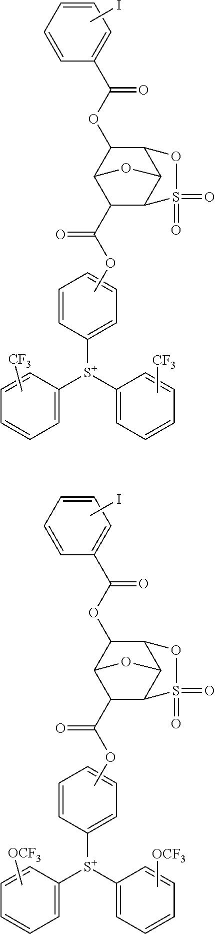

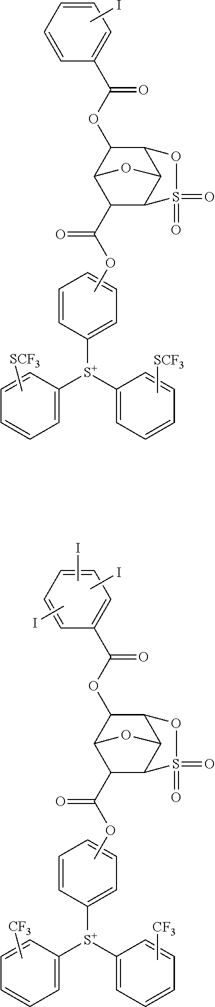

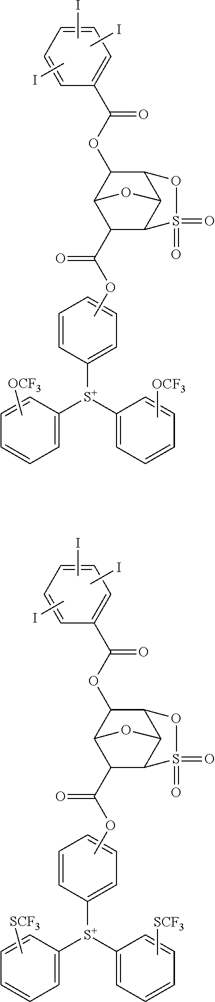

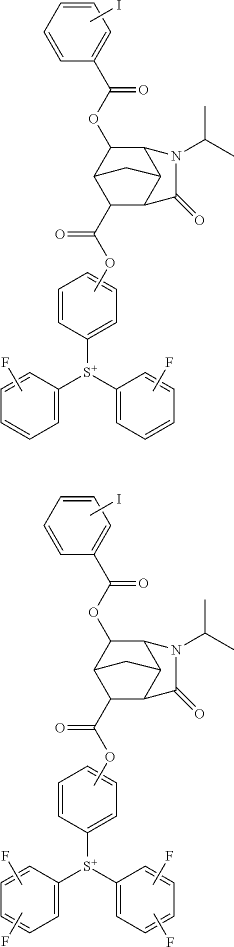

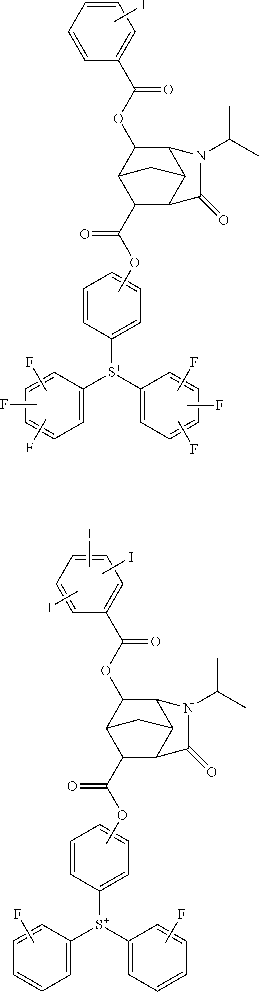

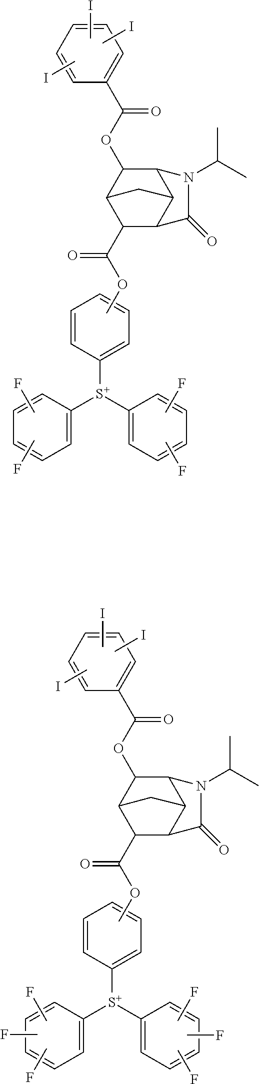

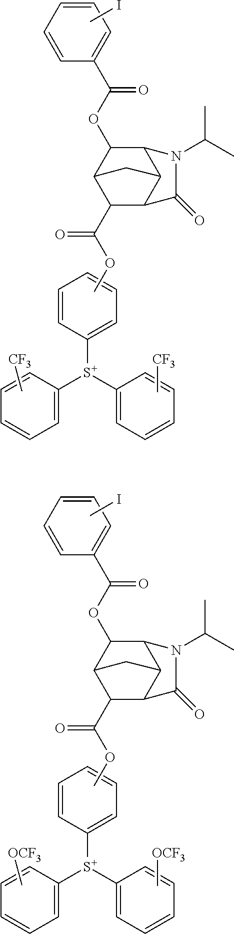

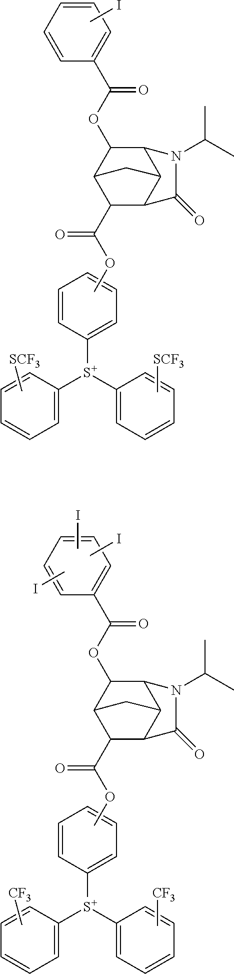

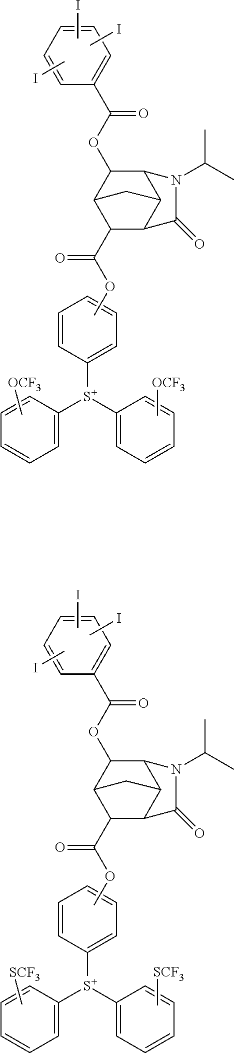

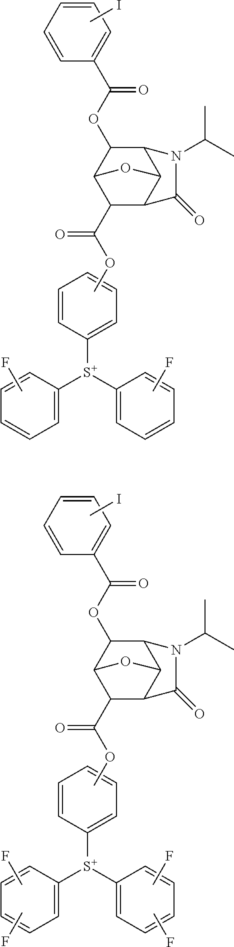

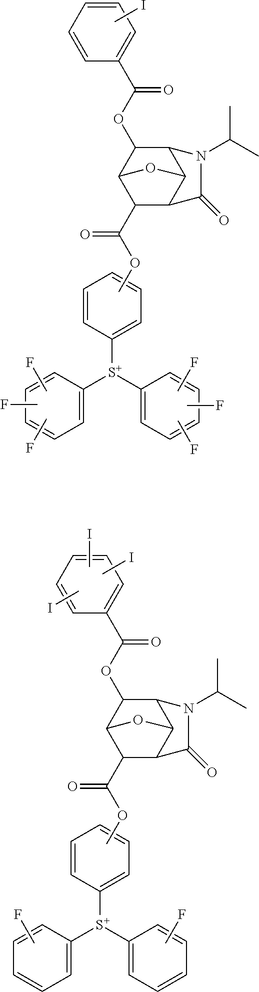

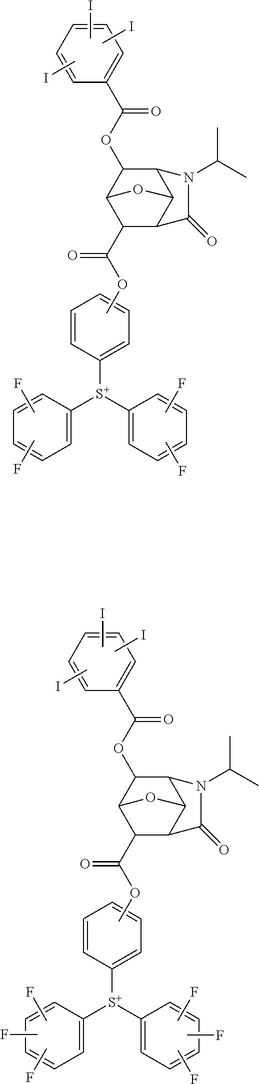

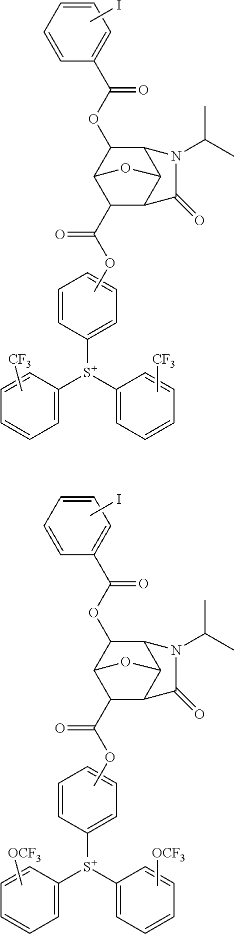

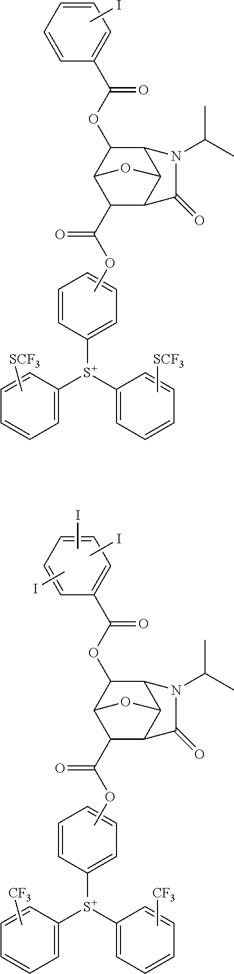

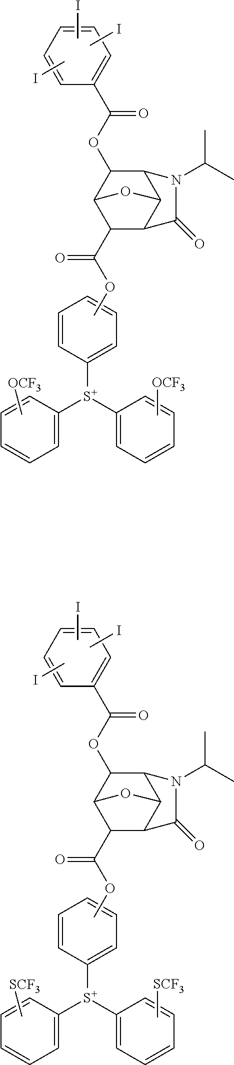

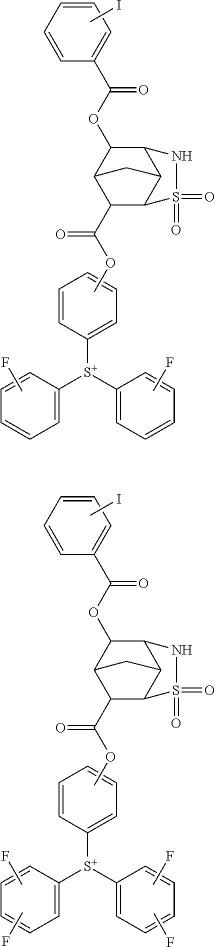

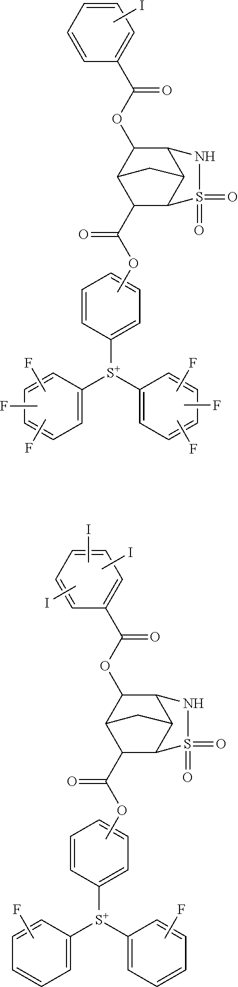

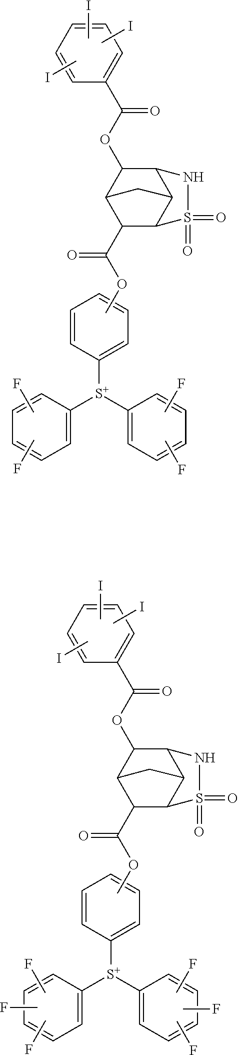

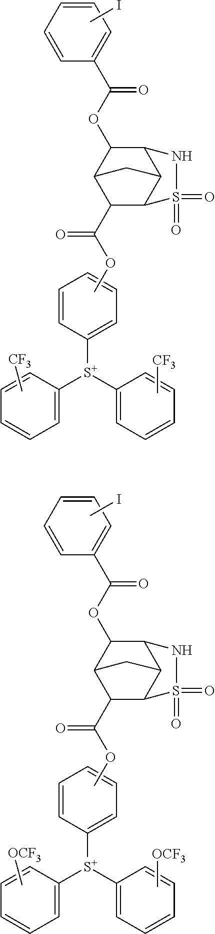

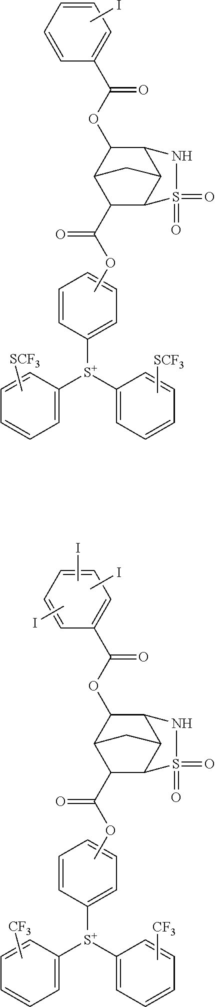

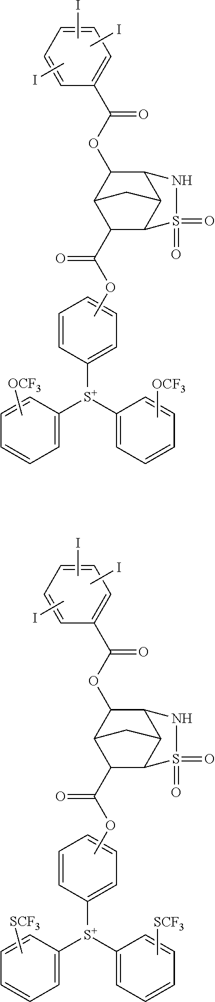

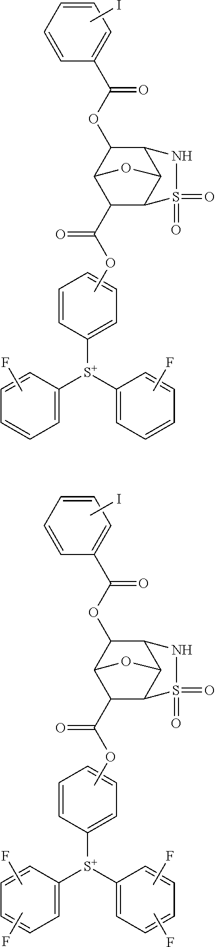

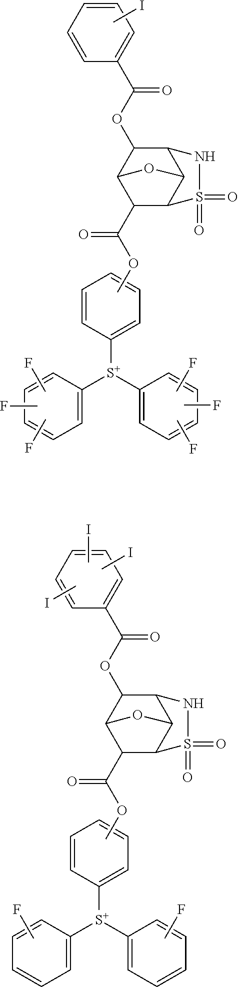

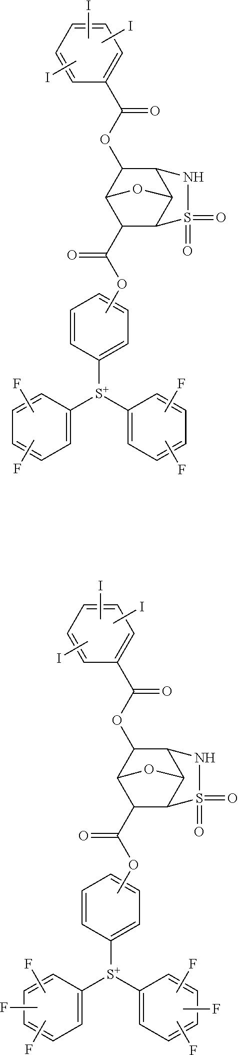

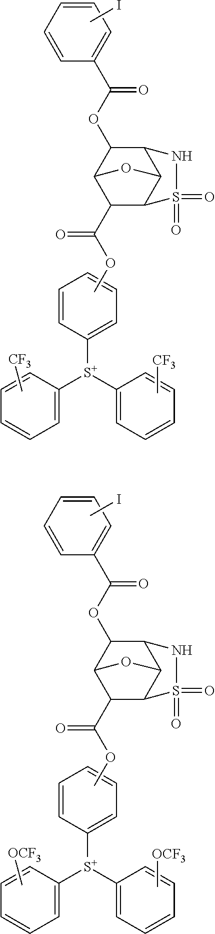

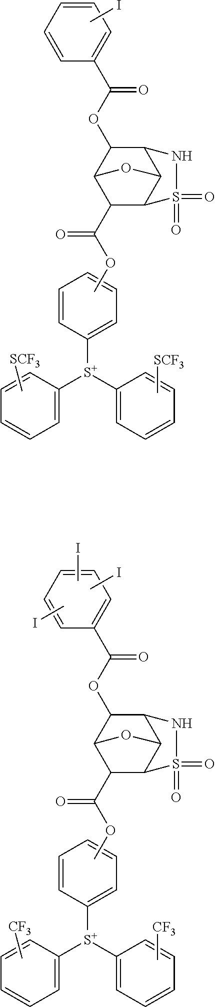

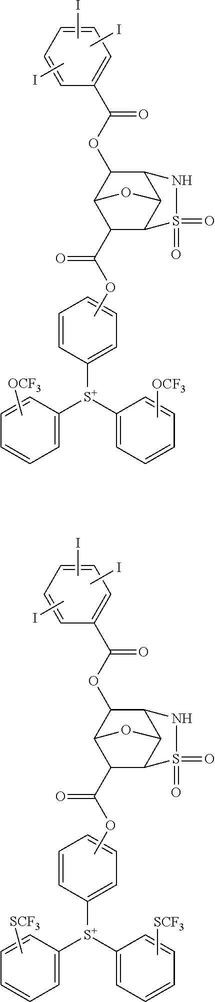

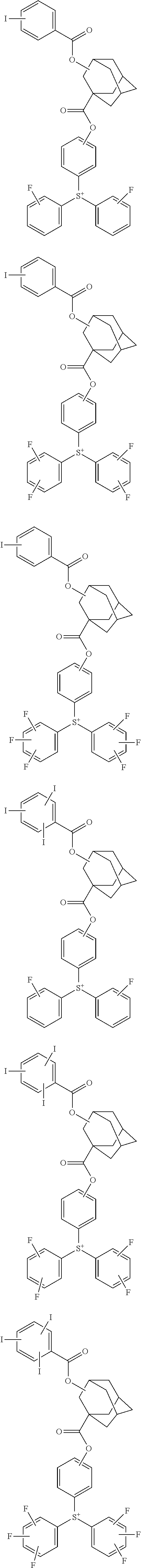

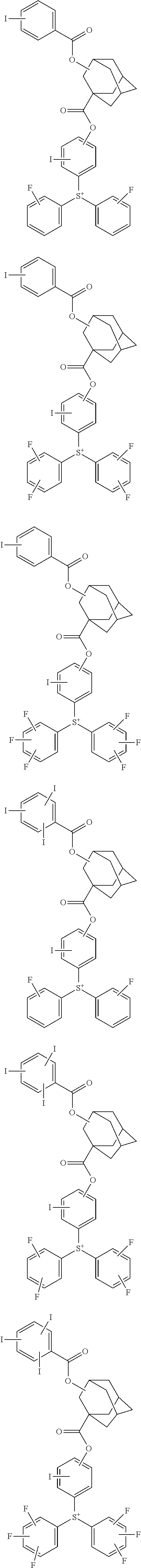

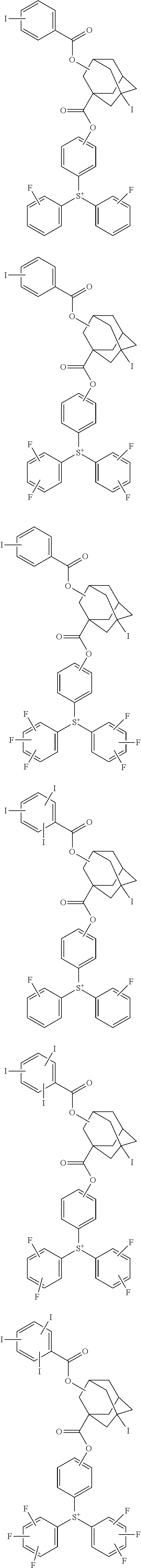

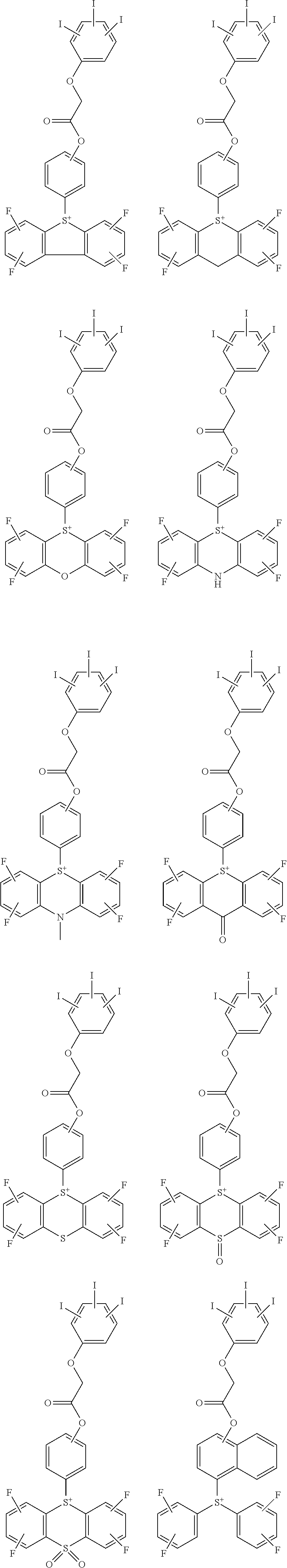

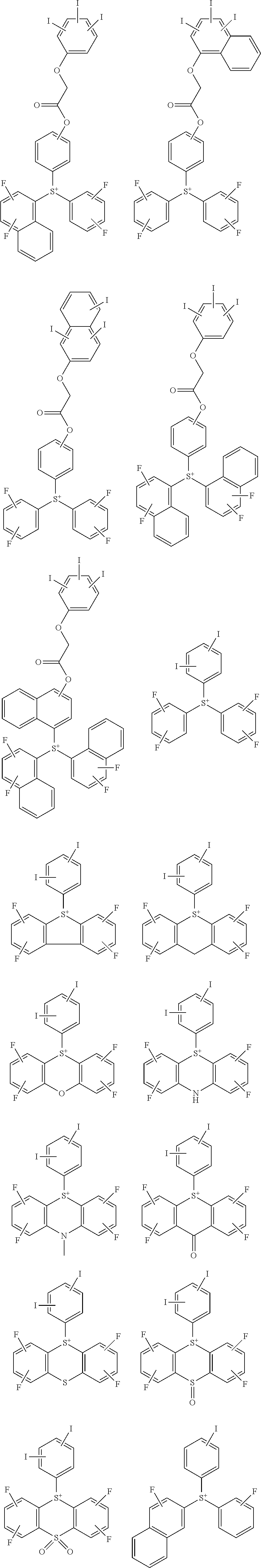

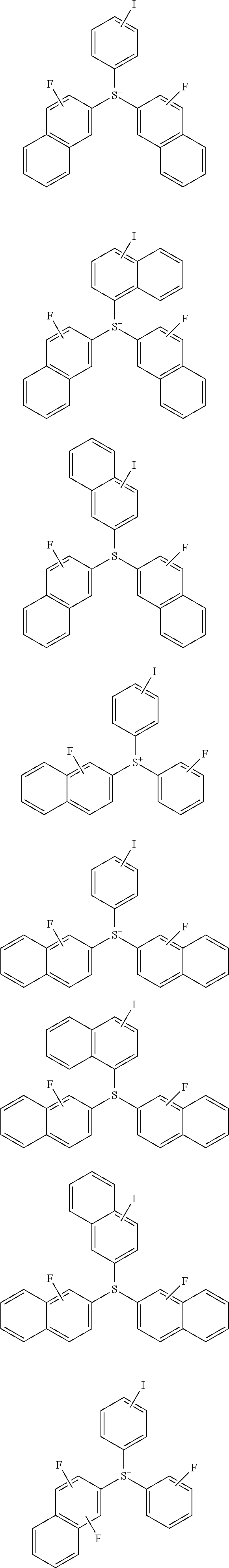

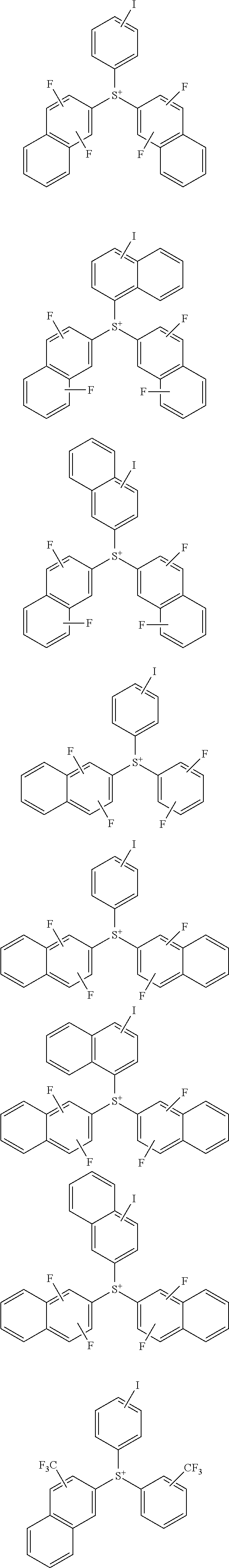

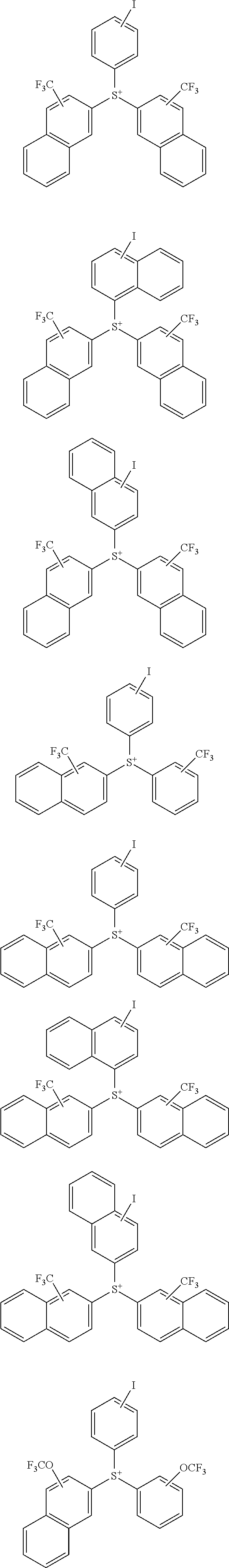

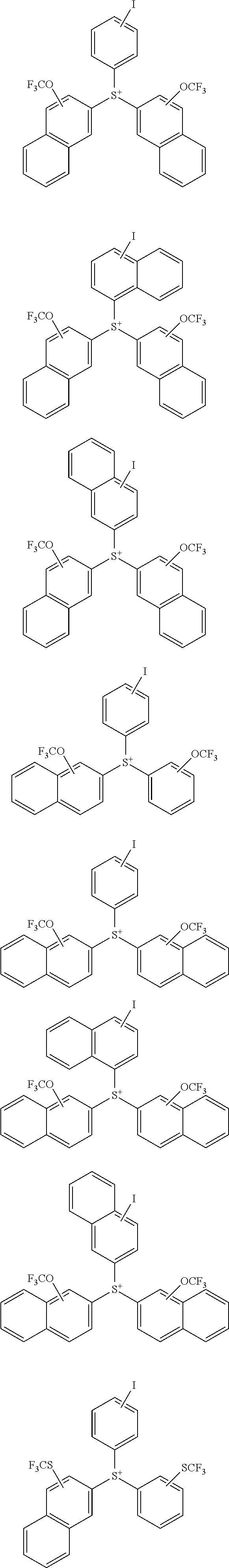

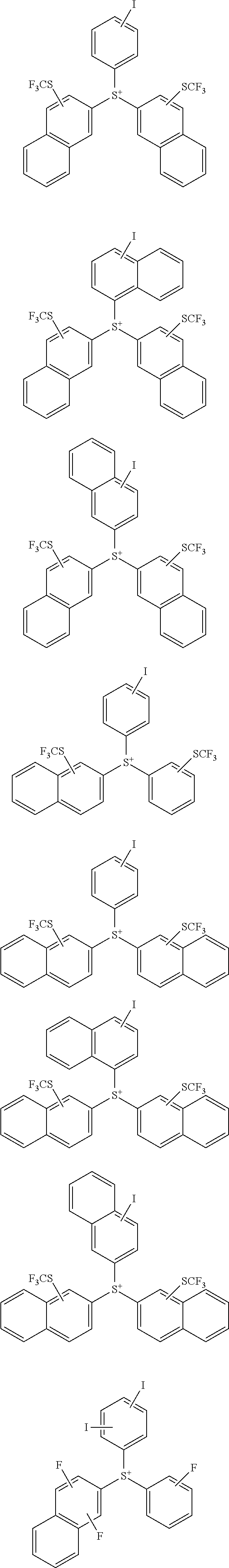

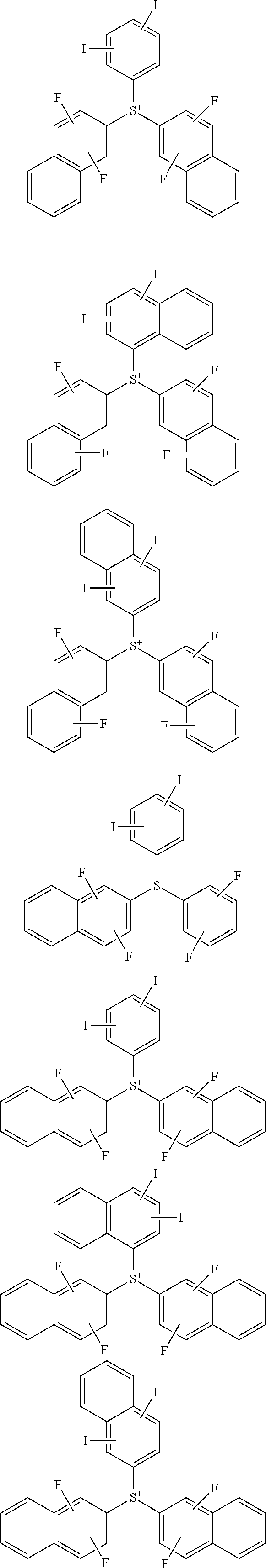

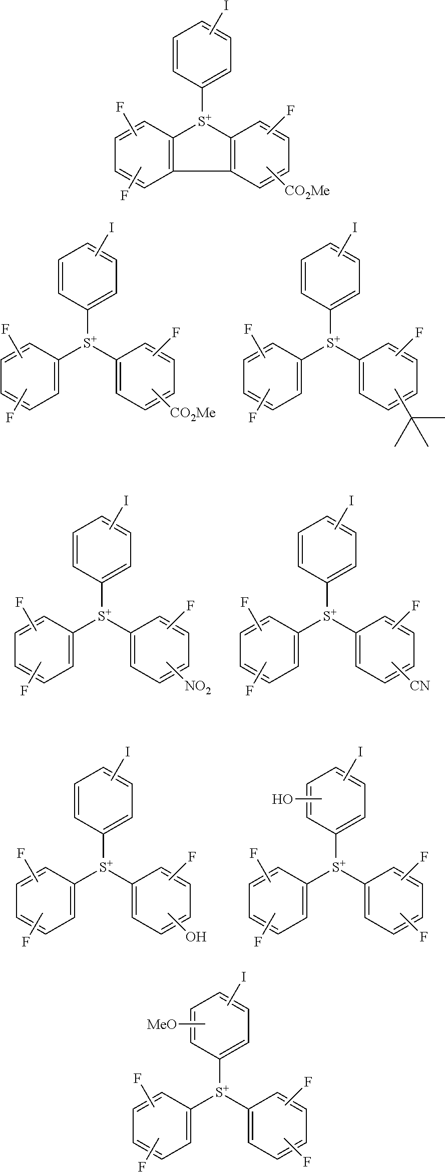

Preferably, the base polymer further comprises repeat units having the formula (e) (hereinafter, also referred to as repeat units (e)).

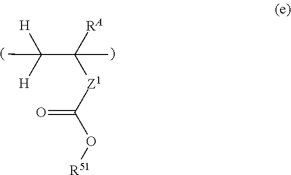

In formula (e), R^{A} is hydrogen, fluorine, methyl group or trifluoromethyl group. Z¹ is a single bond, phenylene group, naphthylene group, *—C(═O)—O—Z¹¹— or *—C(═O)—NH—Z¹¹—, the phenylene group or naphthylene group may be substituted with hydroxy group, nitro group, cyano group, a C₁-C₁₀ saturated hydrocarbyl group which may contain fluorine, a C₁-C₁₀ saturated hydrocarbyloxy group which may contain fluorine, or halogen, * designates a point of attachment to the carbon atom in the backbone, Z¹¹ is a C₁-C₁₀ saturated hydrocarbylene group, phenylene group or naphthylene group, the saturated hydrocarbylene group may contain hydroxy group, ether bond, ester bond or lactone ring, and R¹ is hydrogen, or a C₁-C₂₀ group containing at least one structure selected from a hydroxy group exclusive of phenolic hydroxy group, a cyano group, carbonyl group, carboxy group, ether bond, ester bond, sulfonic ester bond, carbonate bond, lactone ring, sultone ring, and carboxylic anhydride (—C(═O)—O—C(═O)—).

Examples of repeat units (e) are shown below, but not limited thereto. Herein, R^{A} is as defined above.

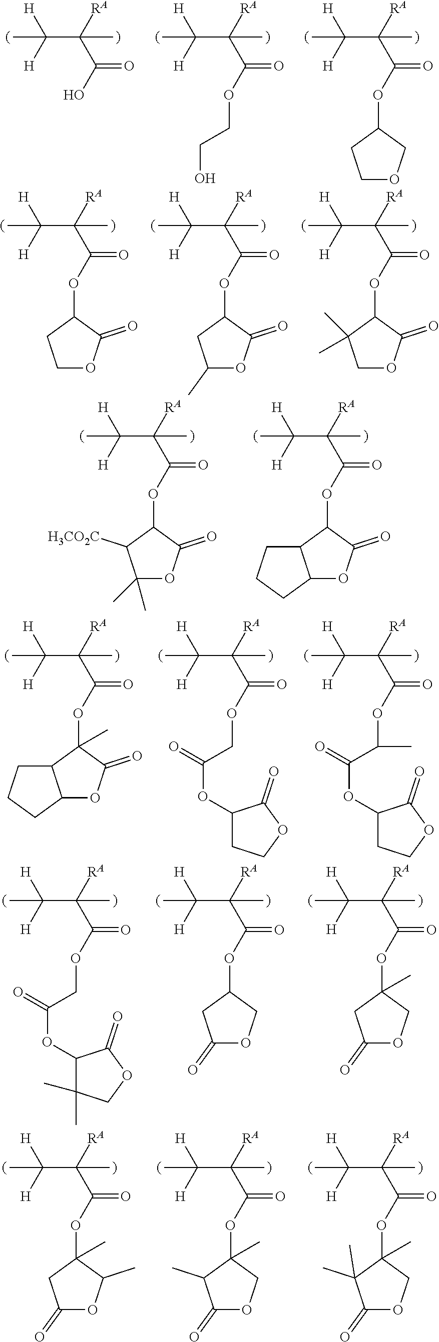

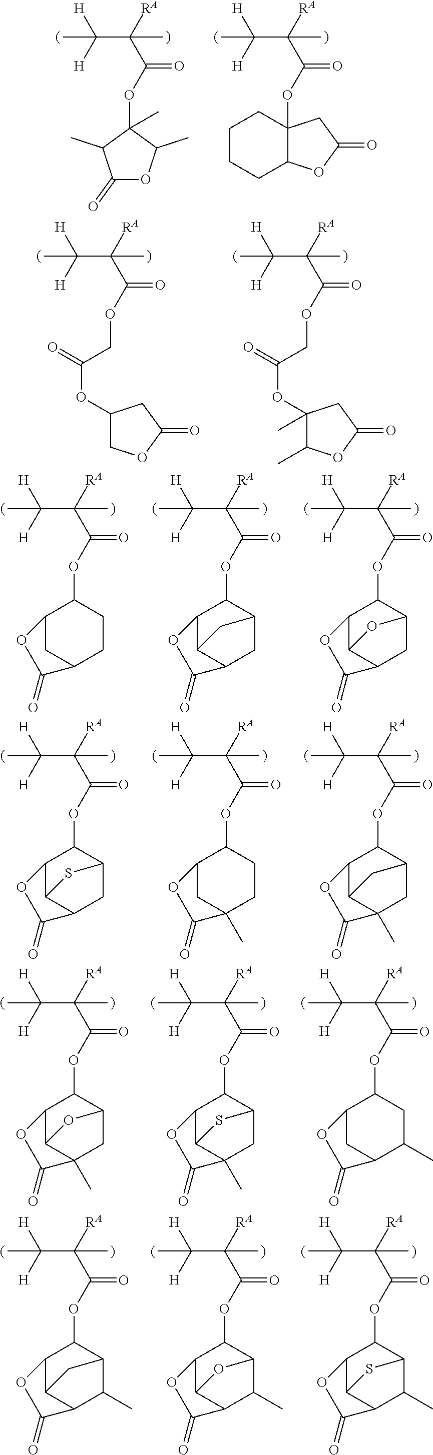

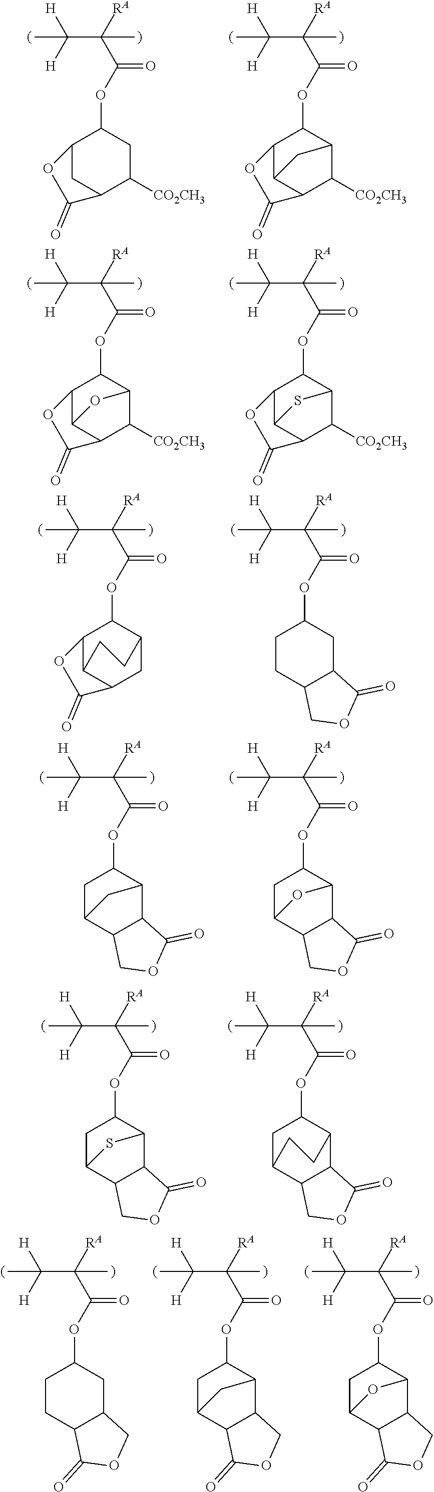

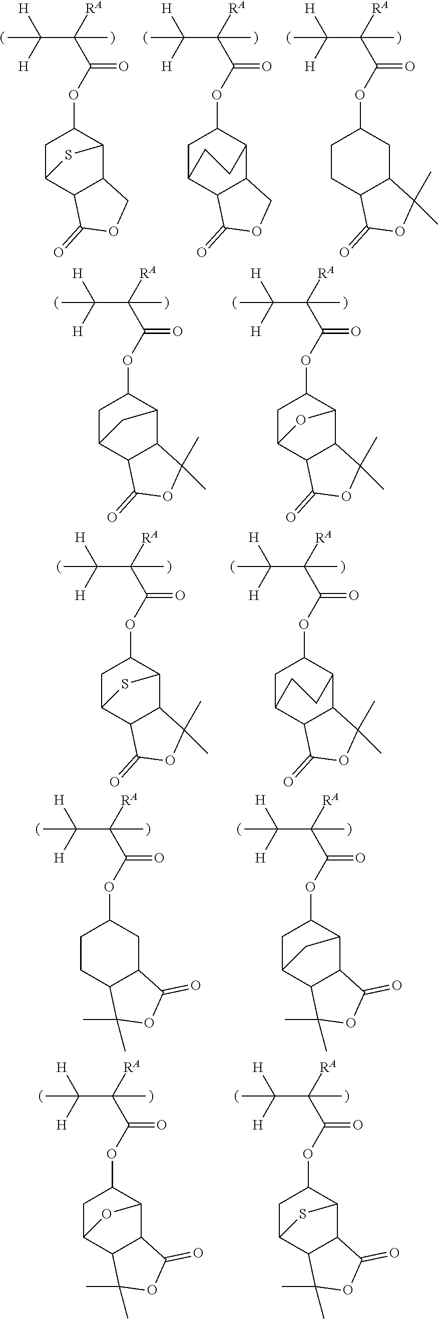

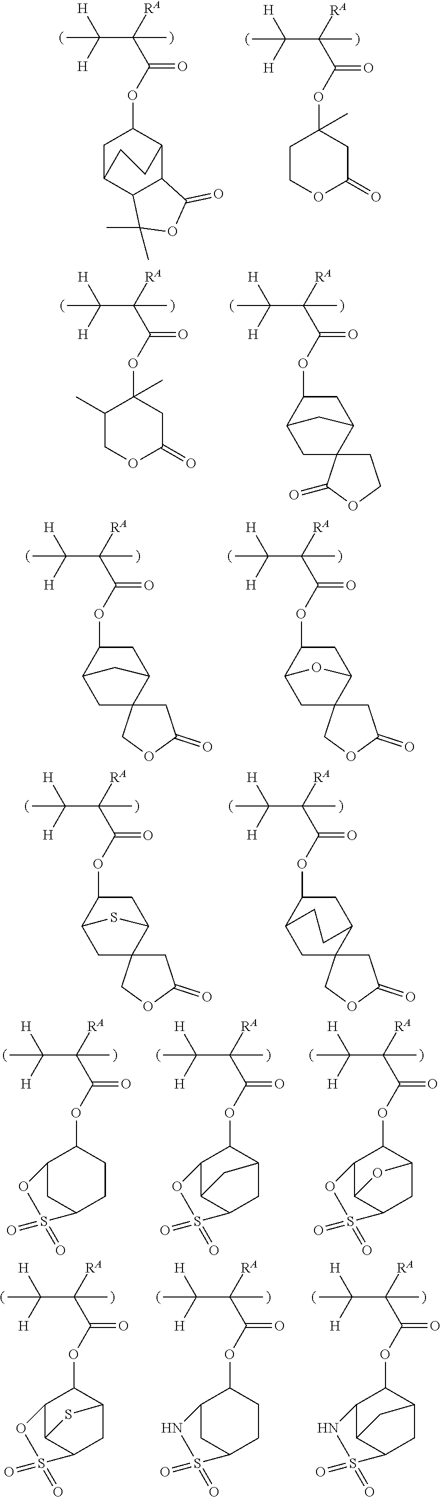

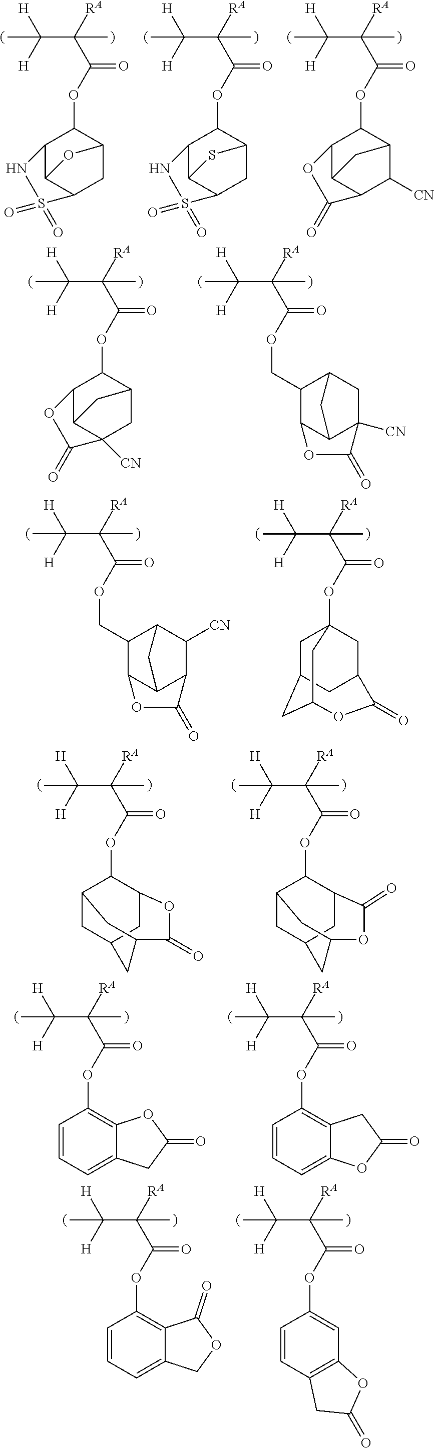

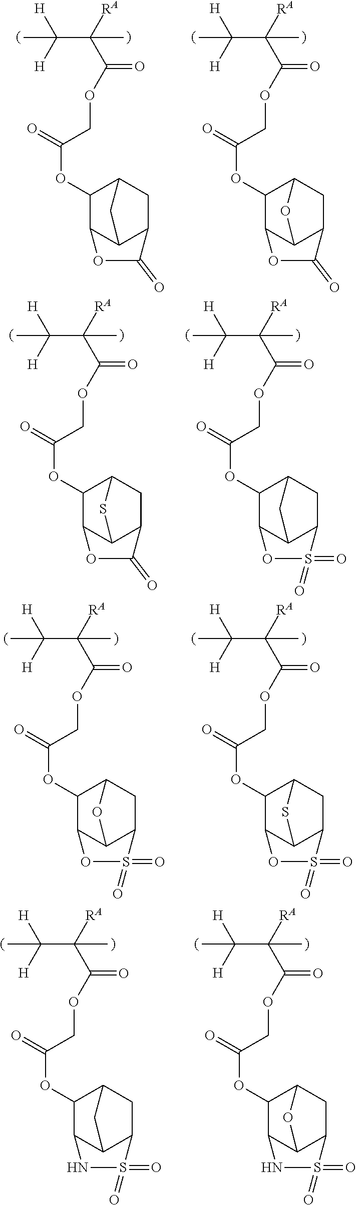

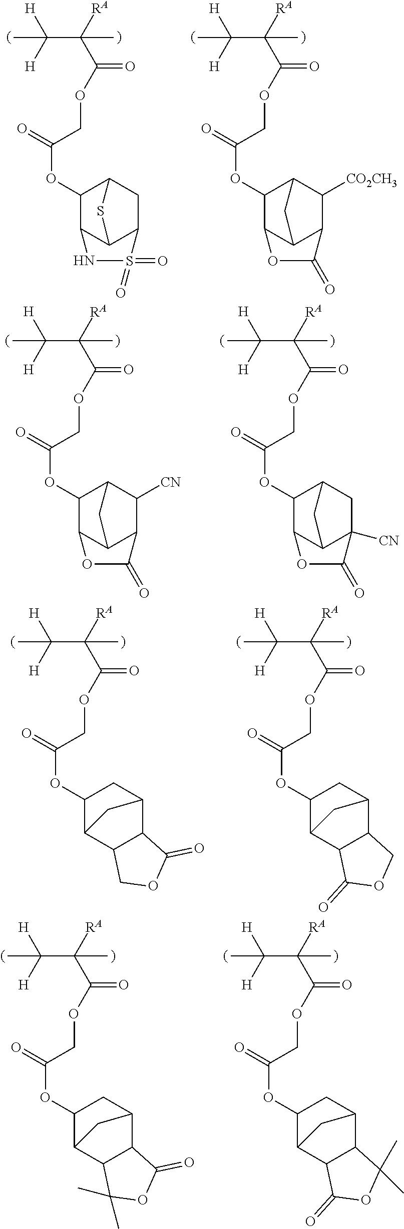

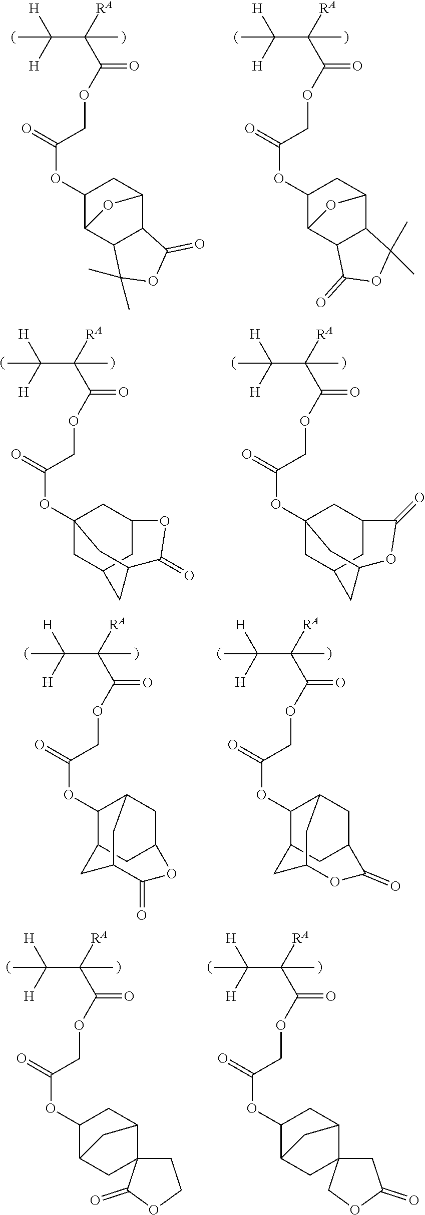

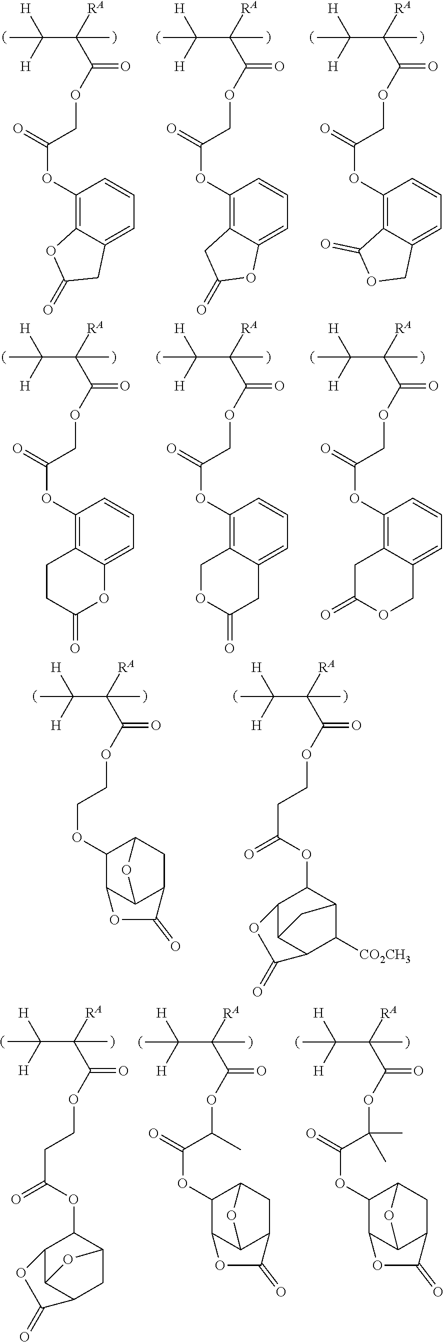

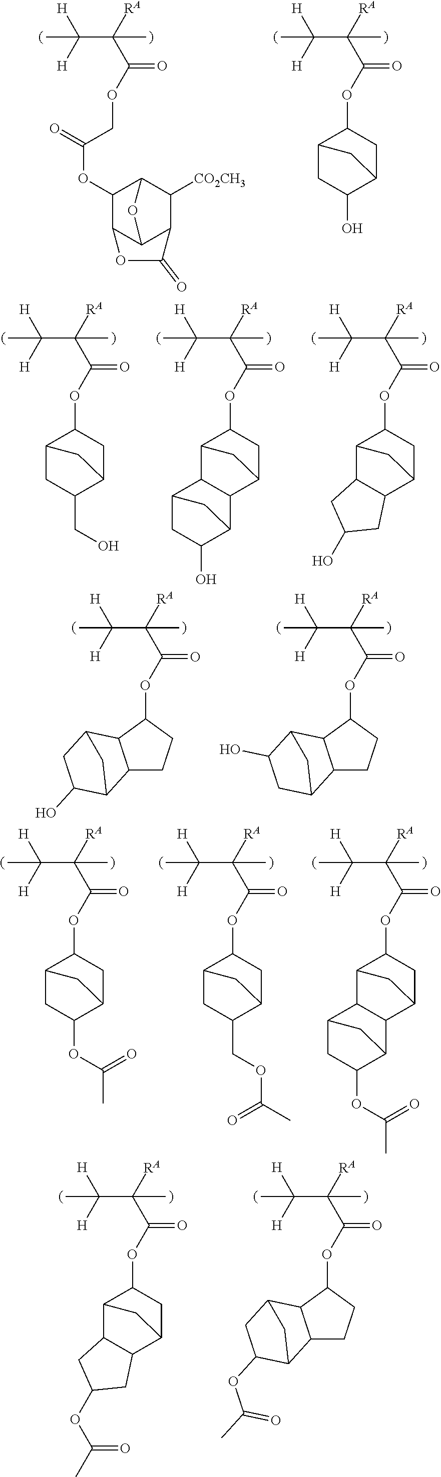

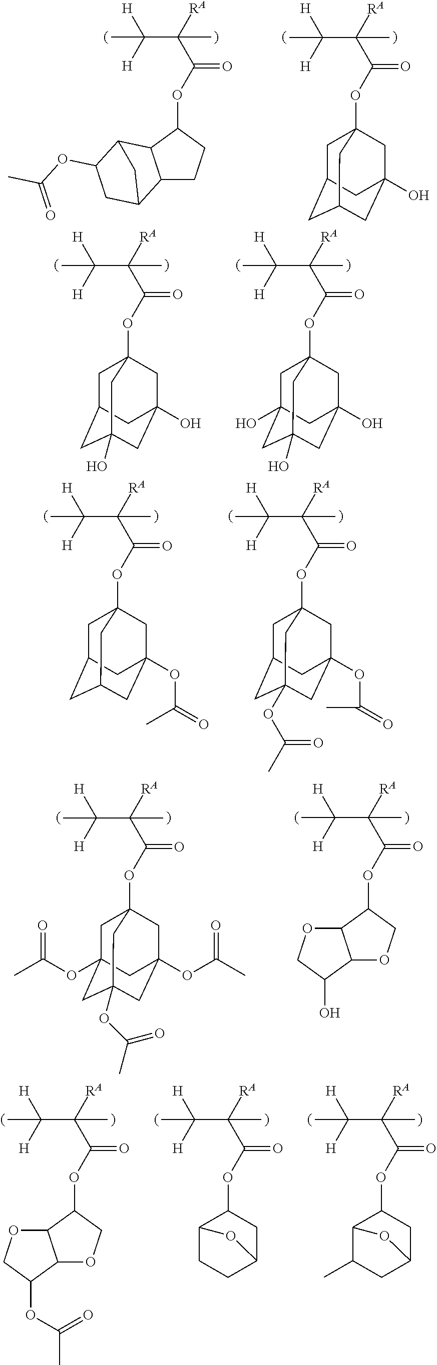

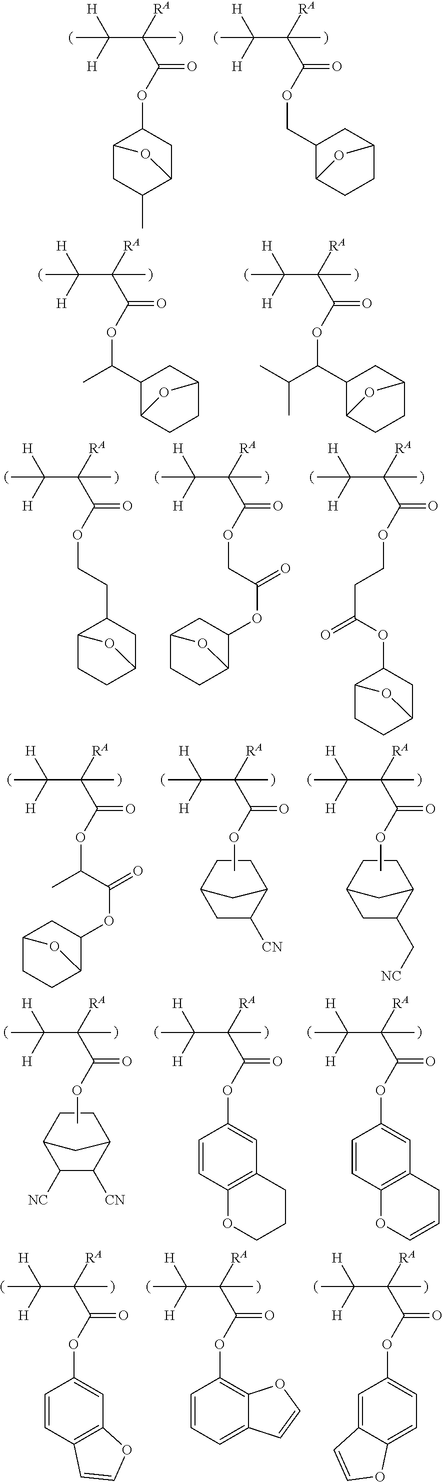

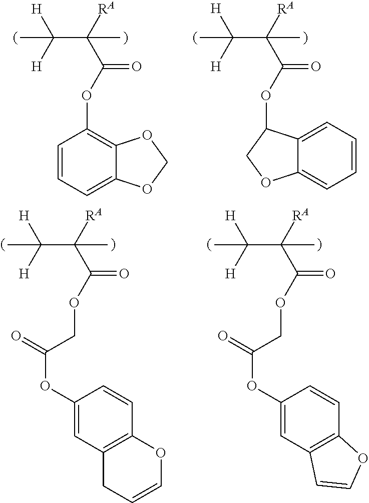

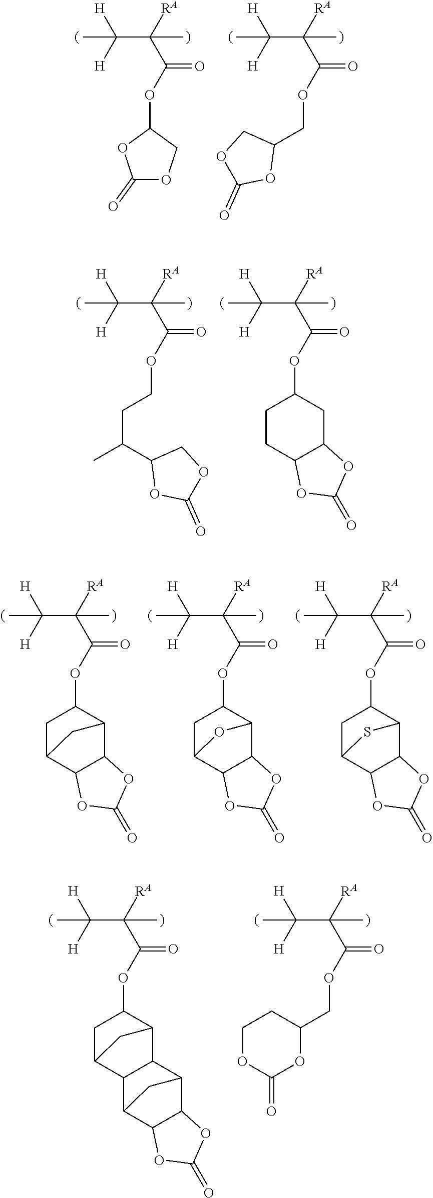

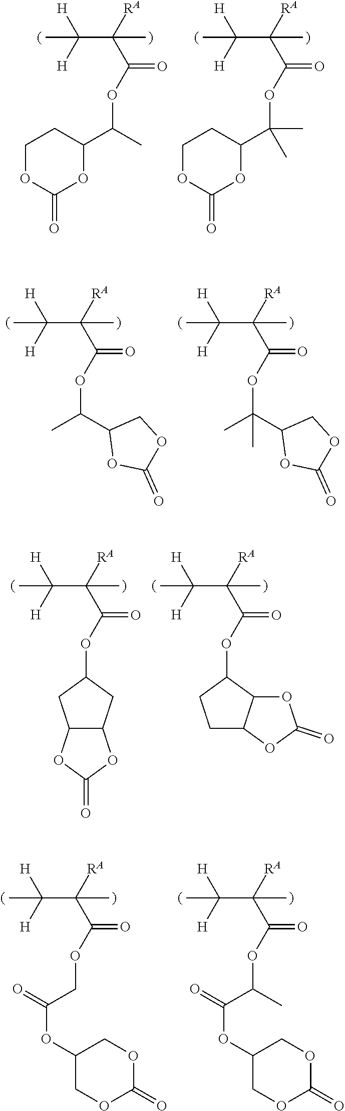

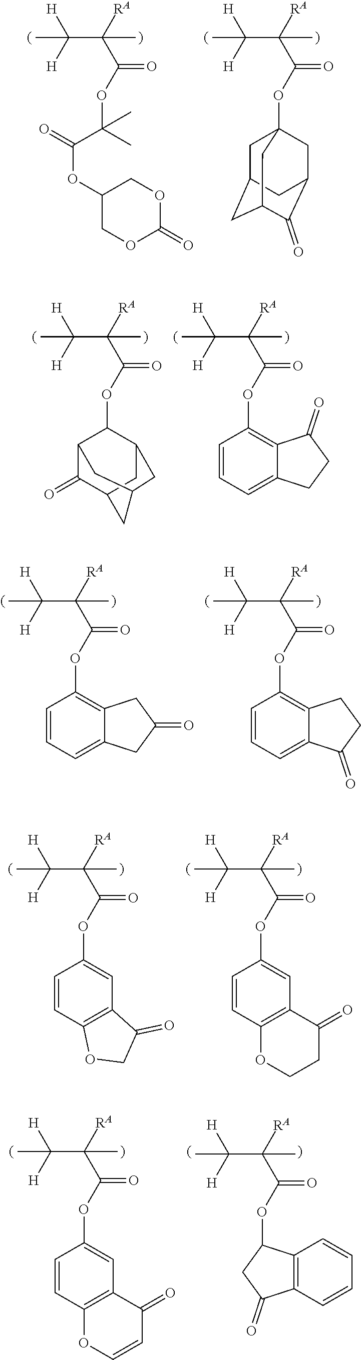

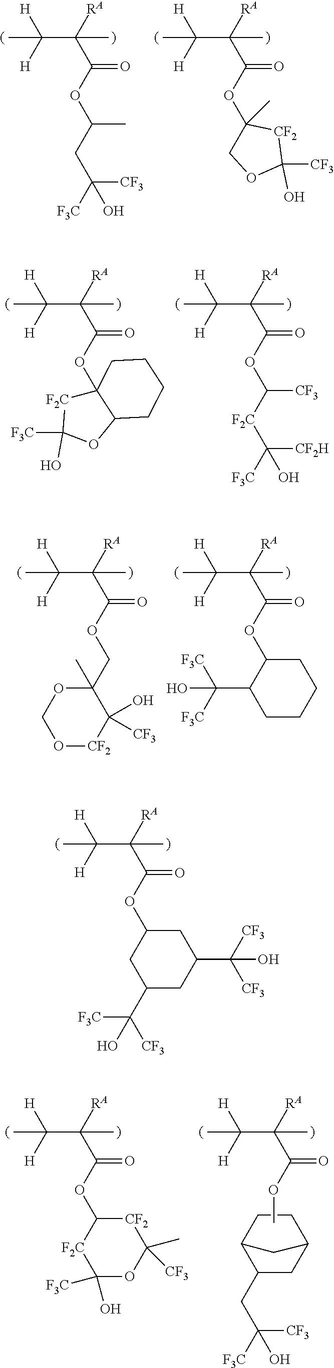

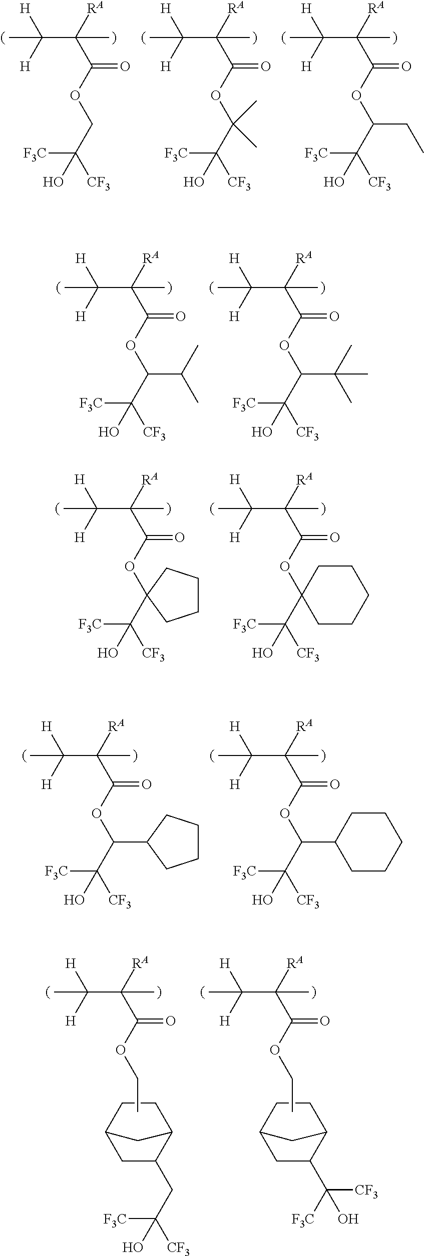

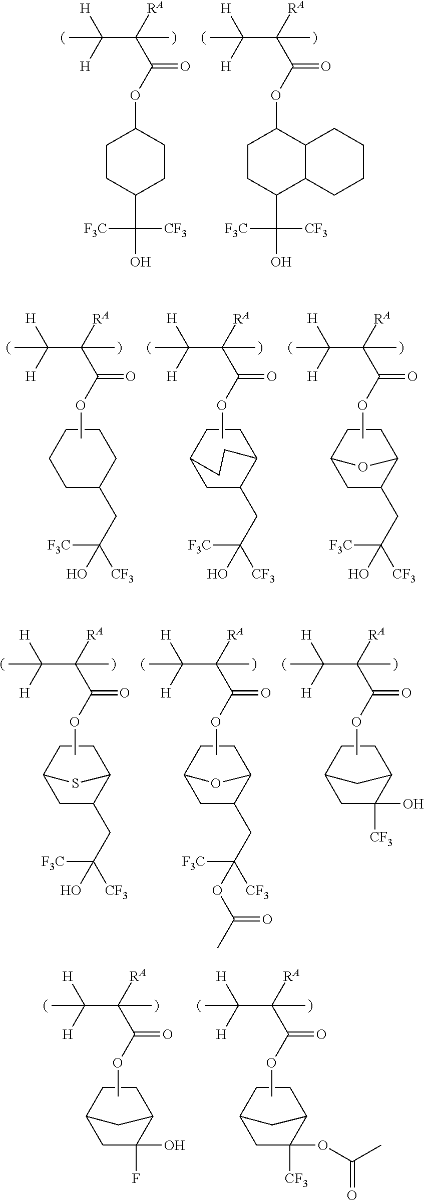

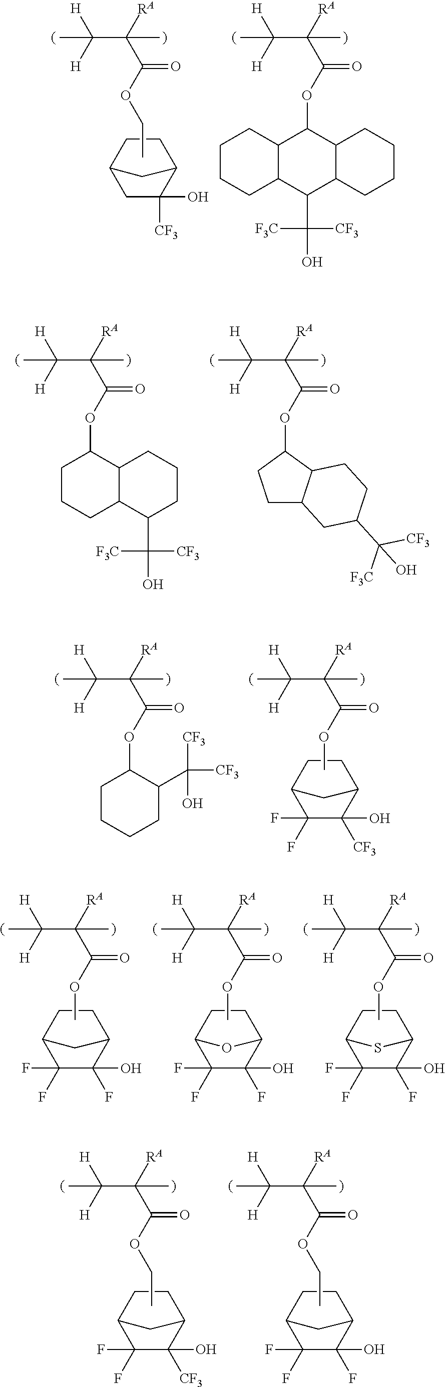

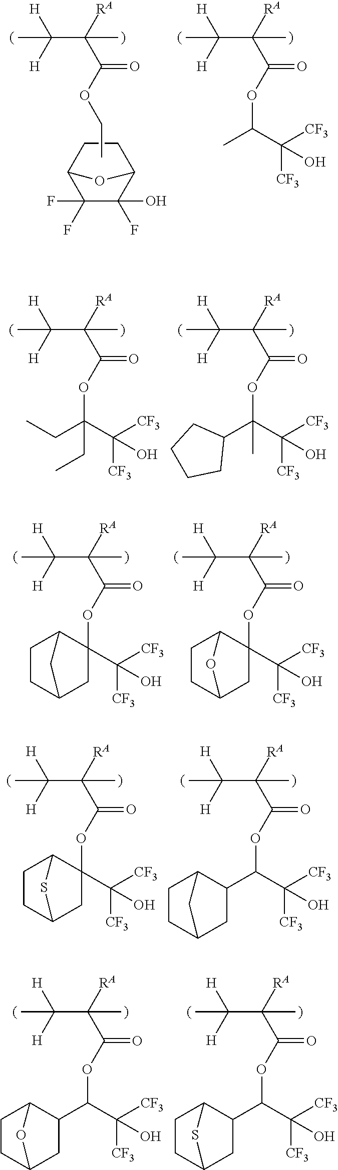

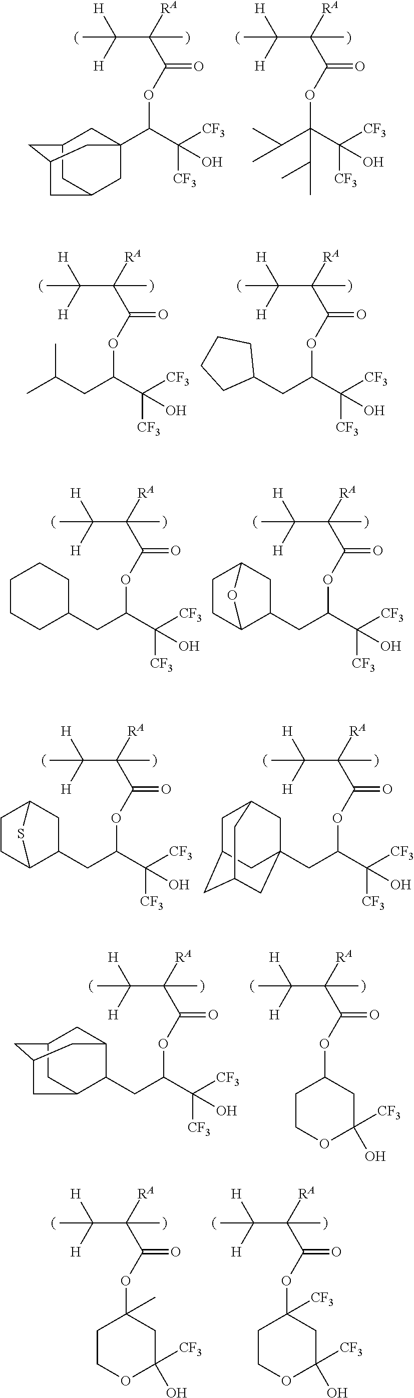

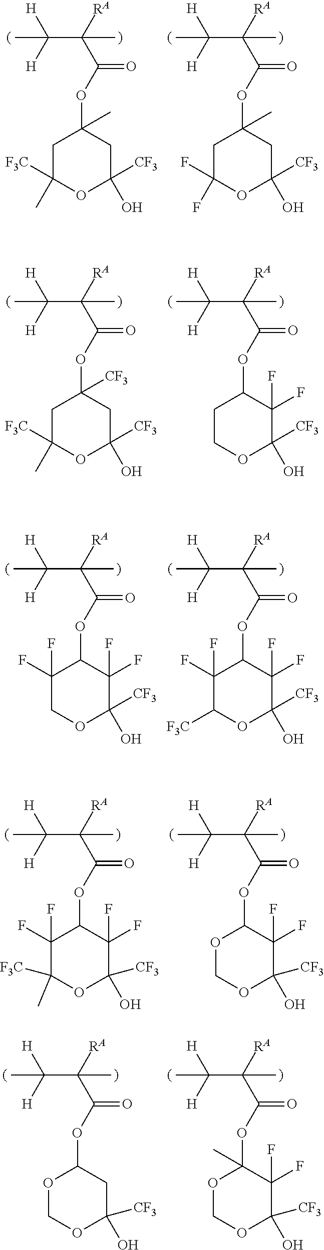

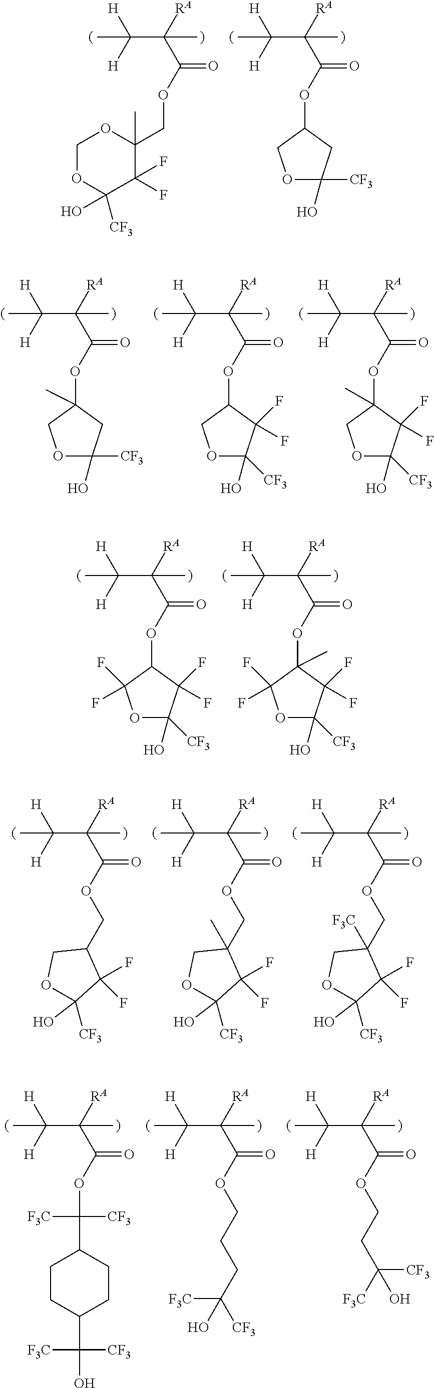

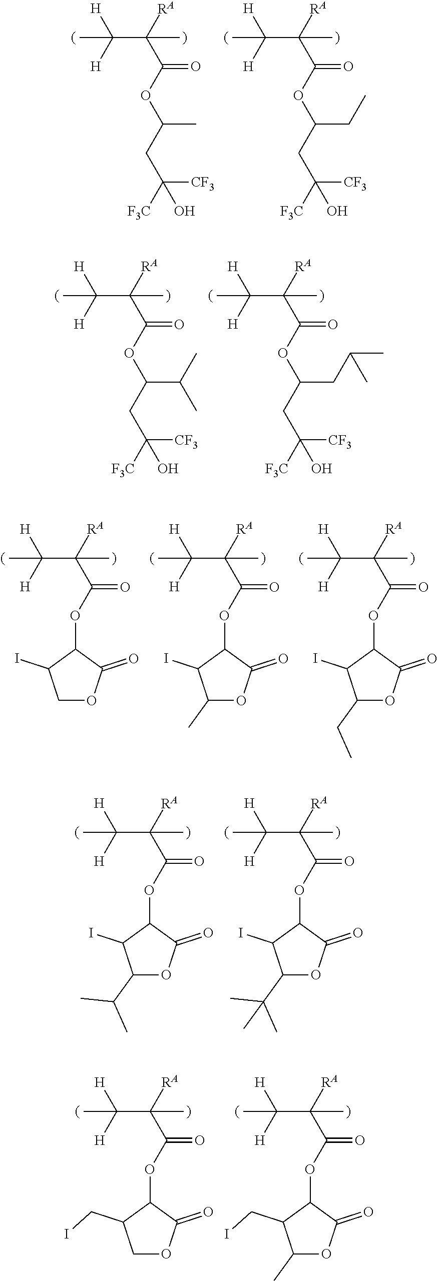

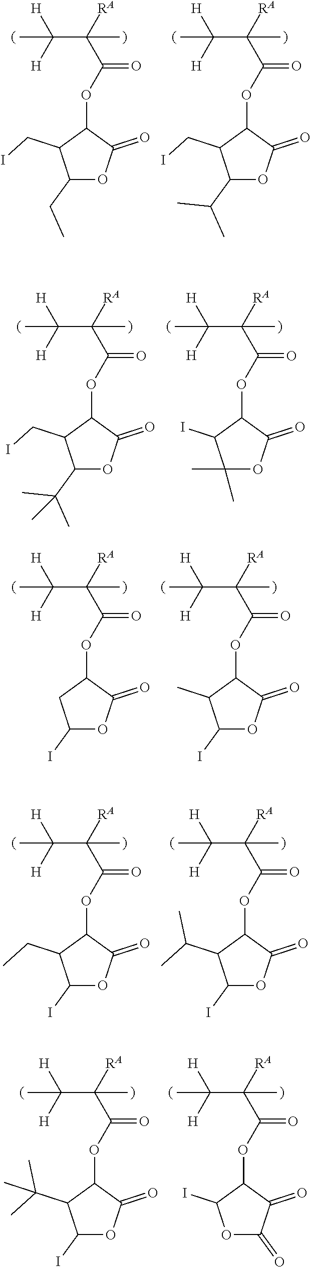

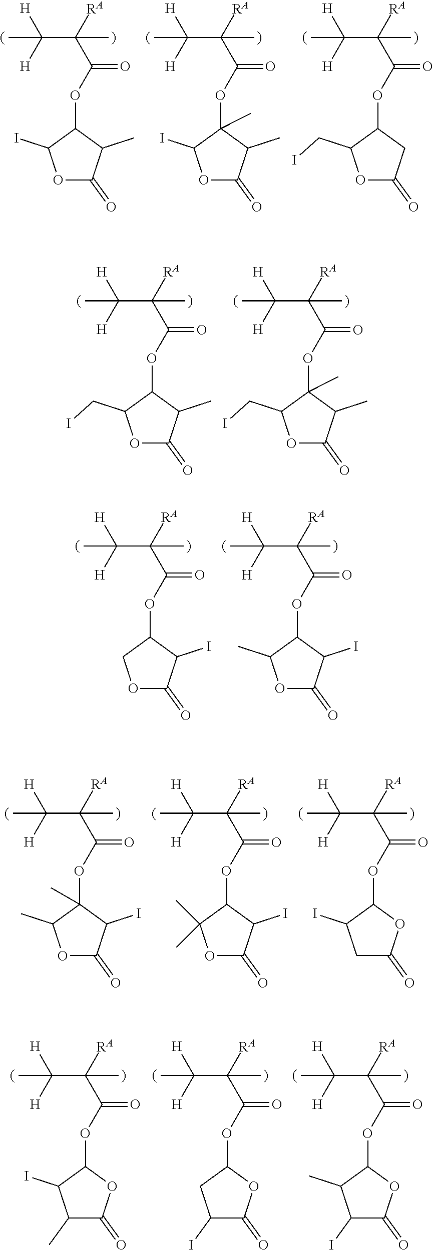

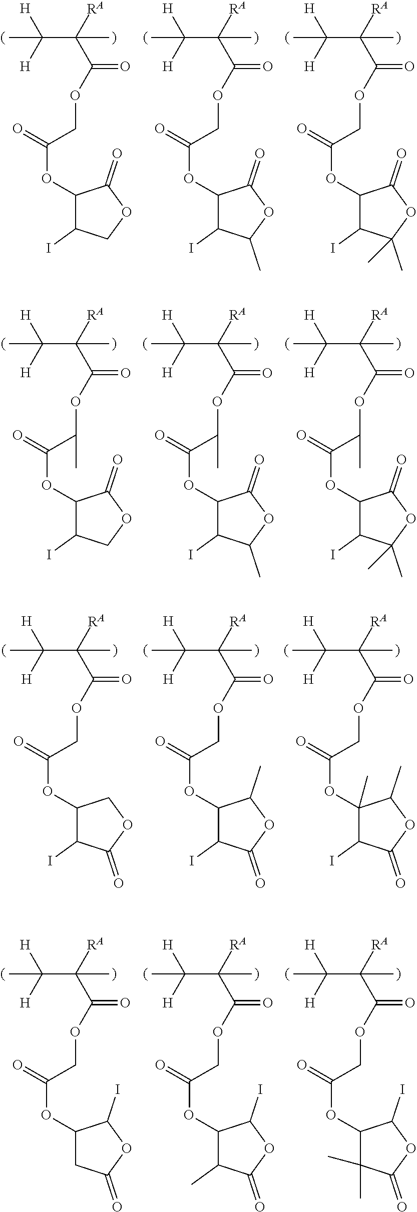

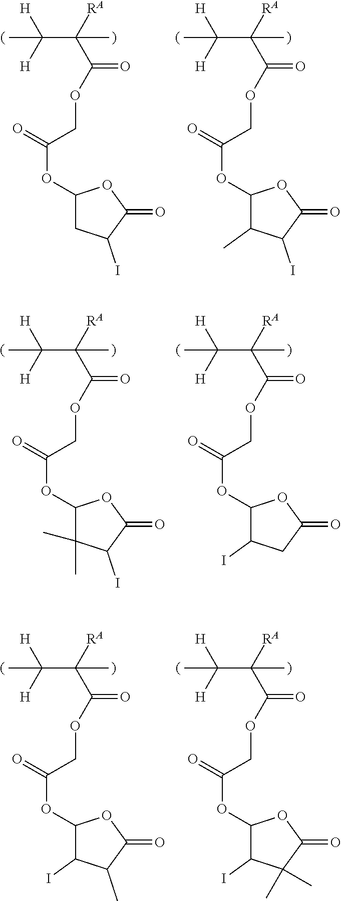

Of the repeat units (e), those units having a lactone ring as the polar group are preferred in the case of ArF lithography, and those units having a phenol site as the polar group are preferred in the case of KrF, EB or EUV lithography.

The polymer may further comprise repeat units of a structure having a hydroxy group protected with an acid labile group (hereinafter, also referred to repeat units (f). The repeat unit (e) is not particularly limited as long as the unit includes one or more structures having a hydroxy group protected with a protective group such that the protective group is decomposed to generate a hydroxy group under the action of acid. Repeat units having the formula (f) are preferred.

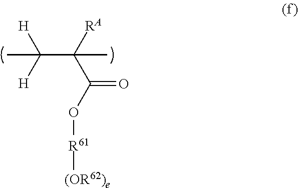

In formula (f), R^{A} is hydrogen, fluorine atom, methyl group, or trifluoromethyl group. R⁶¹ is a C₁-C₃₀ (e+1)-valent hydrocarbon group which may contain a heteroatom. R⁶² is an acid labile group. The subscript e is 1, 2, 3 or 4.

In formula (f), the acid labile group R⁶² is deprotected under the action of acid so that a hydroxy group is generated. The structure of R⁶² is not particularly limited, an acetal structure, ketal structure, alkoxycarbonyl group and alkoxymethyl group having the following formula (f1) are preferred, with the alkoxymethyl group having formula (f1) being more preferred.

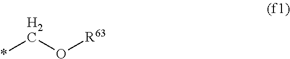

Herein, * designates a point of attachment. R⁶³ is a C₁-C₁₅ hydrocarbyl group.

Examples of the acid labile group R⁶², the alkoxymethyl group having formula (f1), and the repeat units (f) are as described in JP-A 2020-111564 as examples of repeat units (d).

In addition to the foregoing units, the base polymer may further comprise repeat units (g) derived from indene, benzofuran, benzothiophene, acenaphthylene, chromone, coumarin, and norbornadiene, or derivatives thereof. Examples of the monomer from which repeat units (g) are derived are shown below, but not limited thereto.

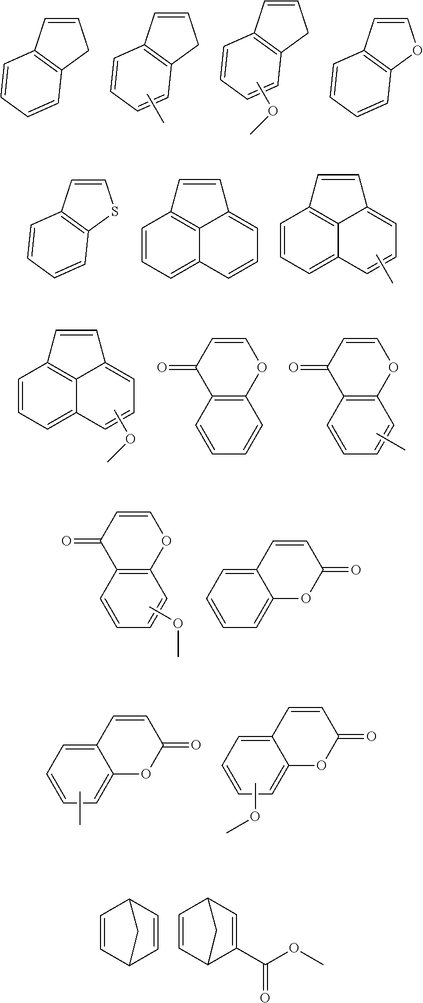

Furthermore, the base polymer may comprise repeat units (h) derived from styrene, indane, vinylpyridine, vinylcarbazole, or derivatives thereof.

In the inventive polymer, a fraction of repeat units (a1), (b1), (b2), (b3), (c), (d), (e), (f), (g) and (h) are preferably 0<a≤0.4, 0<b1≤0.8, 0<b2≤0.8, 0≤b3≤0.6, 0<c≤0.6, 0≤d≤0.4, 0≤e≤0.6, 0≤f≤0.3, 0 g 0.3 and 0 h 0.3, more preferably 0<a≤0.3, 0≤b1≤0.7, 0≤b2≤0.7, 0≤b3≤0.5, 0<c≤0.5, 0≤d≤0.3, 0≤e≤0.5, 0≤f≤0.2, 0≤g≤0.2 and 0≤h≤0.2. The sum of a+b1+b2+b3+c+d+e+f+g is 1.0 or less.

The polymer should preferably have a weight average molecular weight (Mw) in the range of 1,000 to 500,000, and more preferably 3,000 to 100,000. A Mw in the range ensures satisfactory etch resistance and eliminates the risk of resolution being lowered due to a failure to acquire a difference in dissolution rate before and after exposure. In the invention, Mw is a value measured by gel permeation chromatography (GPC) with tetrahydrofuran (THF) or N,N-dimethylformamide (DMF) as a solvent, and calculated as polystyrene.

Since the influence of dispersity (Mw/Mn) becomes stronger as the pattern rule becomes finer, the polymer should preferably have a narrow dispersity (Mw/Mn) of 1.0 to 2.0 in order to provide a resist composition suitable for micropatterning to a small feature size. A Mw/Mn in the range indicates smaller amounts of lower and higher molecular weight polymers and eliminates the risk of leaving foreign matter on the pattern or degrading the pattern profile after exposure and development.

Examples of the method for synthesizing the polymer include a method in which one or more monomers selected from the monomers corresponding to the foregoing repeat units are dissolved in an organic solvent, a radical polymerization initiator is added thereto, and the mixture is heated for polymerization.

Examples of the organic solvent which can be used for polymerization include toluene, benzene, tetrahydrofuran THF, diethyl ether, dioxane, cyclohexane, cyclopentane, methyl ethyl ketone (MEK), propylene glycol monomethyl ether acetate (PGMEA), and γ-butyrolactone (GBL). Examples of the polymerization initiator used herein include 2,2′-azobisisobutyronitrile (AIBN), 2,2′-azobis(2,4-dimethylvaleronitrile), dimethyl-2,2-azobis(2-methylpropionate), 1,1′-azobis(1-acetoxy-1-phenylethane), benzoyl peroxide, and lauroyl peroxide. The amount of the initiator added is preferably 0.01 to 25 mol % based on the total of monomers. The reaction temperature is preferably 50 to 150° C., more preferably 60 to 100° C. The reaction time is preferably 2 to 24 hours, and more preferably 2 to 12 hours from the aspect of production efficiency.

The polymerization initiator may be added to the monomer solution before supply to a reaction vessel, or an initiator solution may be prepared separately from the monomer solution and each solution may be supplied to a reaction vessel independently. Since there is a possibility that the initiator generates a radical in the standby time, by which polymerization reaction takes place to form an ultrahigh molecular weight compound, it is preferred from the standpoint of quality control that the monomer solution and the initiator solution be independently prepared and added dropwise. The acid labile group that has been incorporated in the monomer may be kept as such, or the polymerization may be followed by protection or partial protection. Any of well-known chain transfer agents such as dodecylmercaptan and 2-mercaptoethanol may be used for the purpose of adjusting molecular weight. An appropriate amount of the chain transfer agent is 0.01 to 20 mol % based on the total of monomers to be polymerized.

Where a monomer having a hydroxy group is copolymerized, the hydroxy group may be replaced by an acetal group susceptible to deprotection with acid, typically ethoxyethoxy, prior to polymerization, and the polymerization be followed by deprotection with weak acid and water. Alternatively, the hydroxy group may be replaced by an acetyl, formyl, pivaloyl or similar group prior to polymerization, and the polymerization be followed by alkaline hydrolysis.

When hydroxystyrene or hydroxyvinylnaphthalene is copolymerized, hydroxystyrene or hydroxyvinylnaphthalene and other monomers may be dissolved in an organic solvent, a radical polymerization initiator is added thereto, and the mixture is heated for polymerization. Instead, as alternative method, acetoxystyrene or acetoxyvinylnaphthalene may be used and after polymerization, the acetoxy group is deprotected by alkaline hydrolysis, for thereby converting the polymer product to polyhydroxystyrene or hydroxypolyvinylnaphthalene.

Examples of the base that may be used in alkaline hydrolysis include aqueous ammonia and triethylamine. Preferably the reaction temperature is −20° C. to 100° C., more preferably 0° C. to 60° C. The reaction time is 0.2 to 100 hours, more preferably 0.5 to 20 hours.

The amount of each monomer in the monomer solution is to be appropriately set, for example, so as to achieve the foregoing preferred content ratio of the repeat unit.

The reaction solution resulting from polymerization reaction may be used as the final product. Alternatively, the polymer may be recovered in powder form through a purifying step such as re-precipitation step of adding the reaction solution to a poor solvent and letting the polymer precipitate as powder, after which the polymer powder is used as the final product. It is preferred from the standpoints of operation efficiency and consistent quality to handle a polymer solution which is obtained by dissolving the powder polymer resulting from the purifying step in a solvent, as the final product.

The solvents which can be used herein are described in JP-A 2008-111103, paragraphs [0144]-[0145]. Exemplary solvents include ketones such as cyclohexanone and methyl-2-n-pentyl ketone; alcohols such as 3-methoxybutanol, 3-methyl-3-methoxybutanol, 1-methoxy-2-propanol, 1-ethoxy-2-propanol; ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones such as GBL; alcohols such as diacetone alcohol (DAA); and high-boiling alcohols such as diethylene glycol, propylene glycol, glycerol, 1,4-butanediol, and 1,3-butanediol, which may be used alone or in admixture.

The polymer solution preferably has a polymer concentration of 0.01 to 30 wt %, more preferably 0.1 to 20 wt %.

Prior to use, the reaction solution or polymer solution is preferably filtered through a filter. Filtration is effective for consistent quality because foreign matter and gel which can cause defects are removed.

Suitable materials of which the filter is made include fluorocarbon, cellulose, nylon, polyester, and hydrocarbon base materials. Preferred for the filtering step of a chemically amplified resist composition are filters made of fluorocarbons commonly known as Teflon®, hydrocarbons such as polyethylene and polypropylene, and nylon. While the pore size of the filter may be selected appropriate to comply with the desired cleanness, the filter preferably has a pore size of up to 100 nm, more preferably up to 20 nm. A single filter may be used or a plurality of filters may be used in combination. Although the filtering method may be single pass of the solution, preferably the filtering step is repeated by flowing the solution in a circulating manner. In the polymer preparation process, the filtering step may be carried out any times, in any order and in any stage. The reaction solution as polymerized or the polymer solution may be filtered, preferably both are filtered.

### Chemically Amplified Resist Composition

### (A) Base Polymer

The inventive chemically amplified resist composition comprises (A) a base polymer containing the polymer defined above.

The polymer may be used alone or as a mixture of two or more polymers which are different in compositional ratio, Mw and/or Mw/Mn. In addition to the polymer defined above, the base polymer (A) may contain a hydrogenated product of ring-opening metathesis polymerization polymer, which is described in JP-A 2003-66612.

### (B) Organic Solvent

The inventive chemically amplified resist composition may comprise (B) an organic solvent. The (B) organic solvent is not particularly limited as long as the component (A) and components described later are soluble therein. Examples of the organic solvent include ketones such as cyclopentanone, cyclohexanone and methyl-2-n-pentyl ketone; alcohols such as 3-methoxybutanol, 3-methyl-3-methoxybutanol, 1-methoxy-2-propanol, 1-ethoxy-2-propanol; keto-alcohols such as DAA, ethers such as PGME, ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; and lactones such as GBL, which may be used alone or in admixture.

Of the foregoing organic solvents, 1-ethoxy-2-propanol, PGMEA, cyclohexanone, GBL, DAA and mixtures thereof are preferred because the base polymer (A) is most soluble therein.

The content of the organic solvent (B) in the inventive chemically amplified resist composition is preferably 200 to 5,000 parts by weight, more preferably 400 to 3,500 parts by weight per 80 parts by weight of the base polymer (A). The organic solvent (B) may be used alone or in admixture.

### (C) Quencher

The inventive chemically amplified resist composition may comprise (C) a quencher. In the invention, the quencher refers to a compound capable of trapping the acid, which is generated by the photoacid generator in the chemically amplified resist composition upon light exposure, to prevent the acid from diffusing to the unexposed region and to assist in forming the desired pattern.

Examples of the quencher (C) include onium salts having the following formulae (1) and (2).

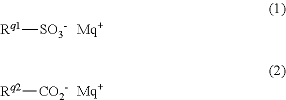

In formula (1), R^{q1} is hydrogen atom or a C₁-C₄₀ hydrocarbyl group which may contain a heteroatom, exclusive of the hydrocarbyl group in which the hydrogen atom bonded to the carbon atom at α-position of the sulfo group is substituted by fluorine atom or fluoroalkyl group. In formula (2), R^{q2} is hydrogen, or a C₁-C₄₀ hydrocarbyl group which may contain a heteroatom.

Examples of the C₁-C₄₀ hydrocarbyl group R^{q1} include C₁-C₄₀ alkyls such as methyl, ethyl, n-propyl, isopropyl, n-butyl, isobutyl, sec-butyl, tert-butyl, n-pentyl, tert-pentyl, n-hexyl, n-octyl, 2-ethylhexyl, n-nonyl and n-decyl groups; C₃-C₄₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2.6}]decyl and adamantyl groups; and C₆-C₄₀ aryl groups such as phenyl, naphthyl and anthracenyl groups. Some or all of hydrogen atoms of the hydrocarbyl group may be replaced by a group containing a heteroatom such as oxygen, sulfur, nitrogen or halogen, some constituent —CH₂— of the hydrocarbyl group may be replaced by a group containing a heteroatom such as oxygen, sulfur or nitrogen, and as a result, the hydrocarbyl group may contain a hydroxy group, fluorine, chlorine, bromine, iodine, a cyano group, a carbonyl group, an ether bond, an ester bond, a sulfonic ester bond, a carbonate bond, a lactone ring, a sultone ring, carboxylic anhydride (—C(═O)—O—C(═O)—), a haloalkyl group, or the like.

Examples of the hydrocarbyl group R^{q2} include those exemplified above for R^{q1}, fluorinated saturated hydrocarbyl groups such as trifluoromethyl and trifluoroethyl groups, and fluorinated aryl groups such as pentafluorophenyl and 4-trifluoromethylphenyl groups.

Examples of the anion in the onium salt having formula (1) are shown below, but not limited thereto.

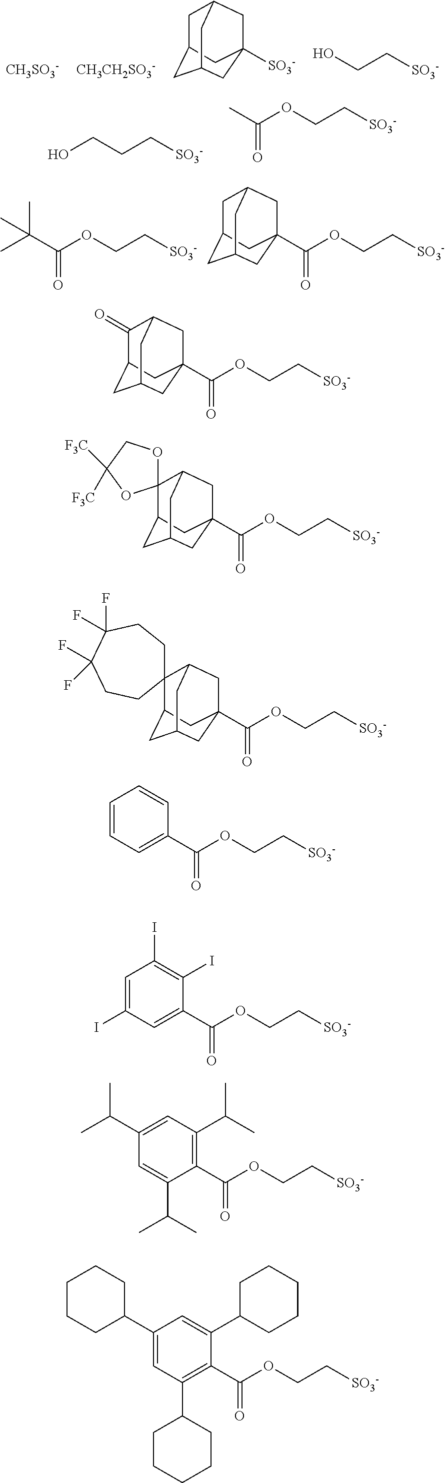

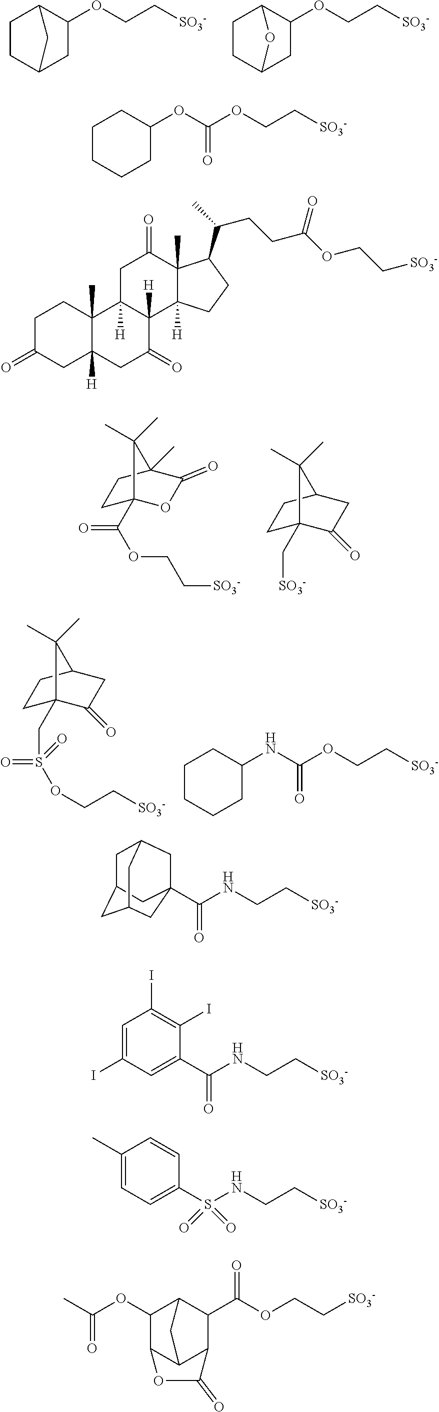

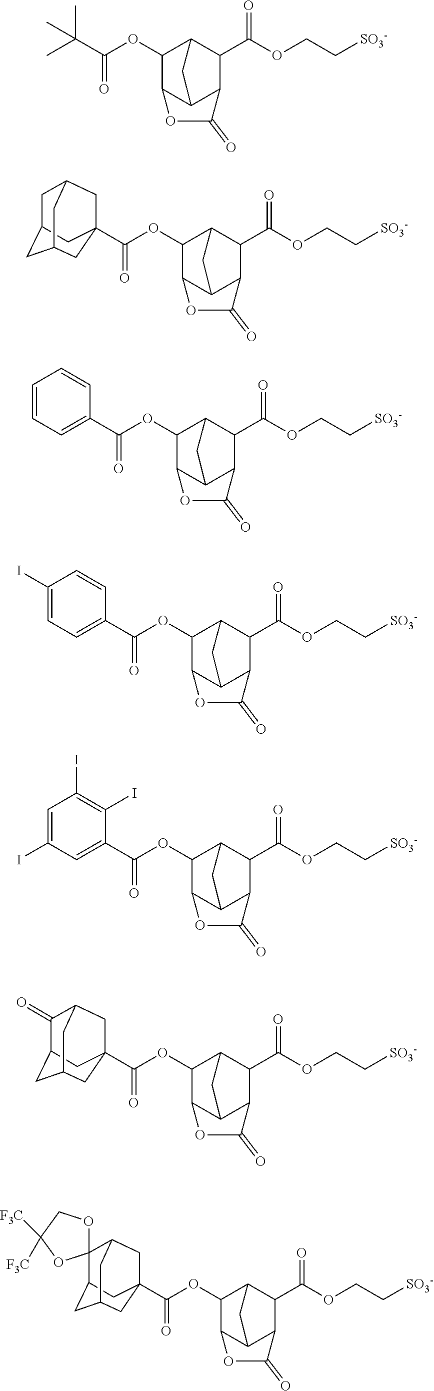

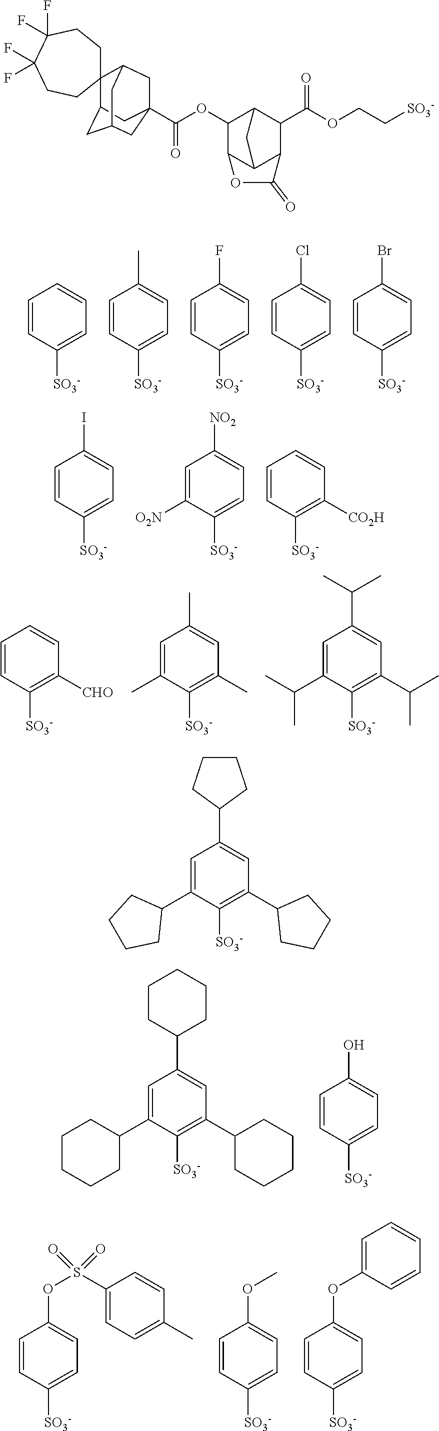

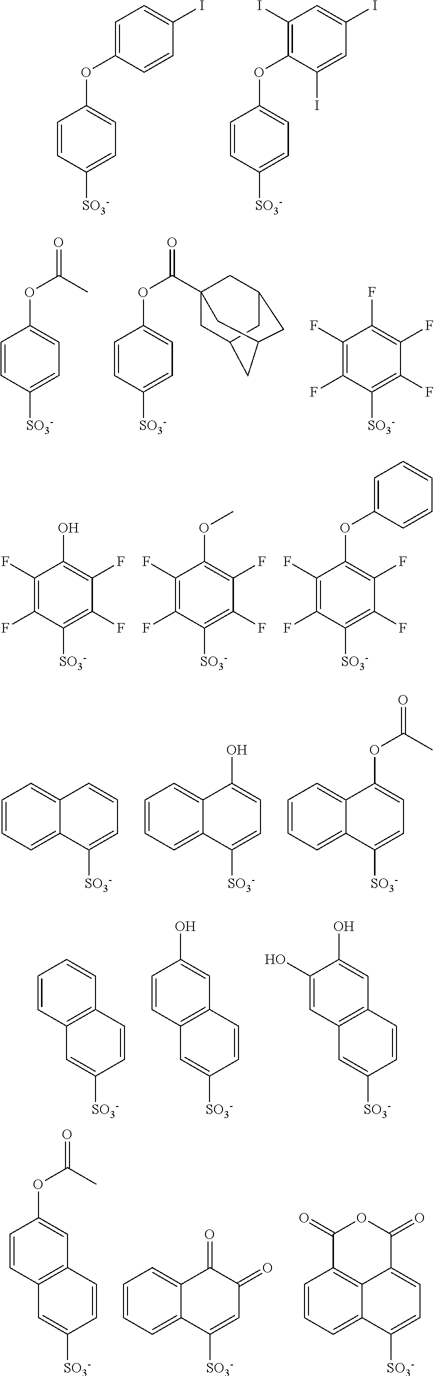

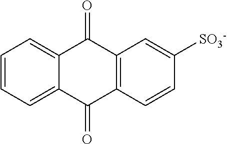

Examples of the anion in the onium salt having formula (2) are shown below, but not limited thereto.

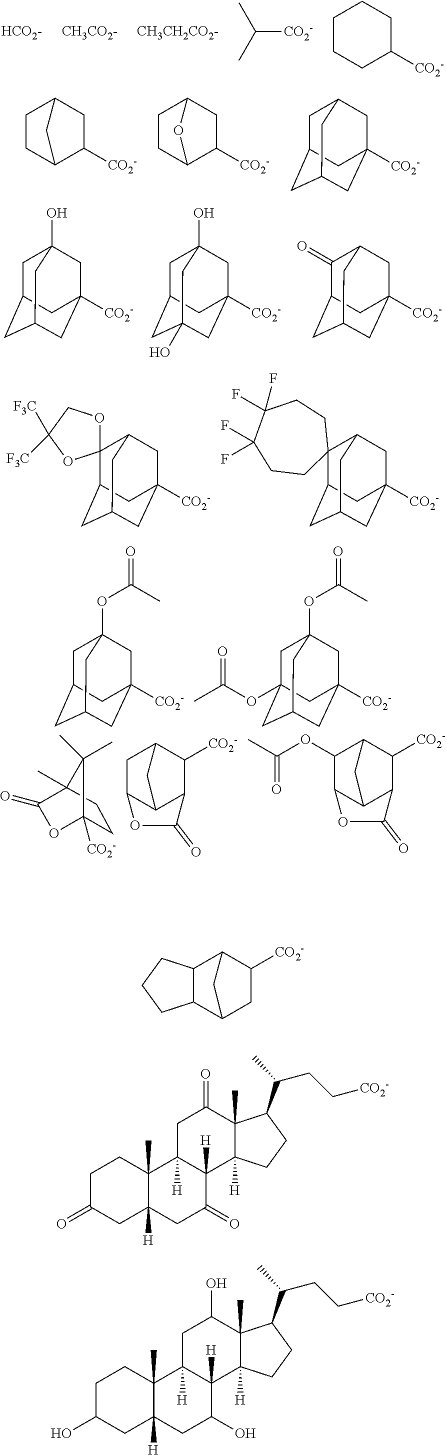

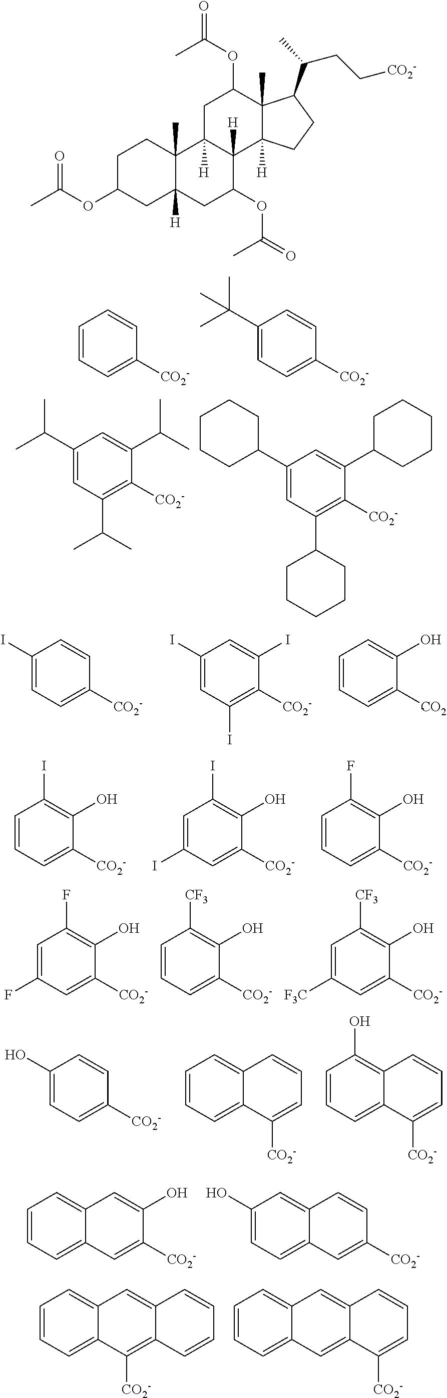

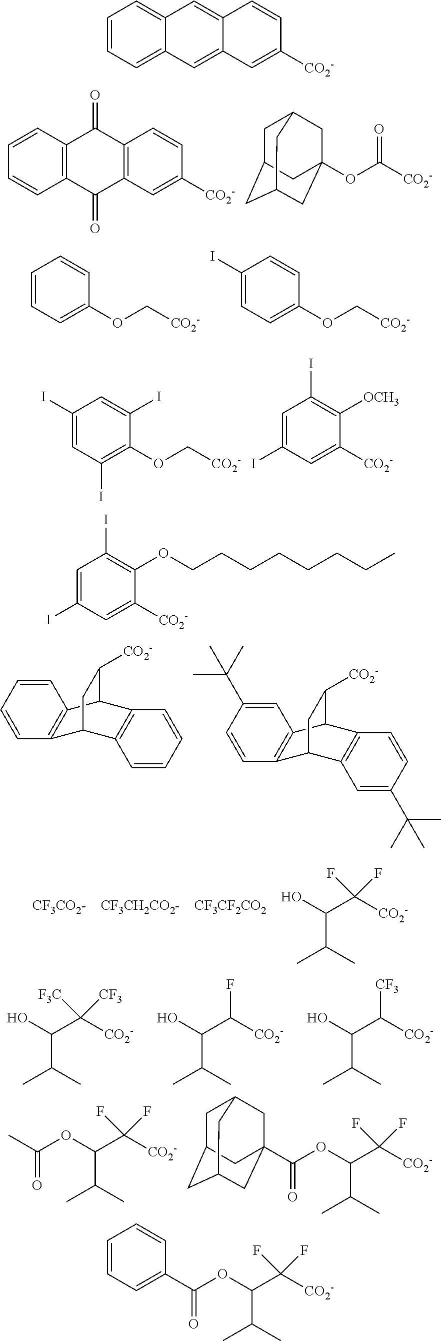

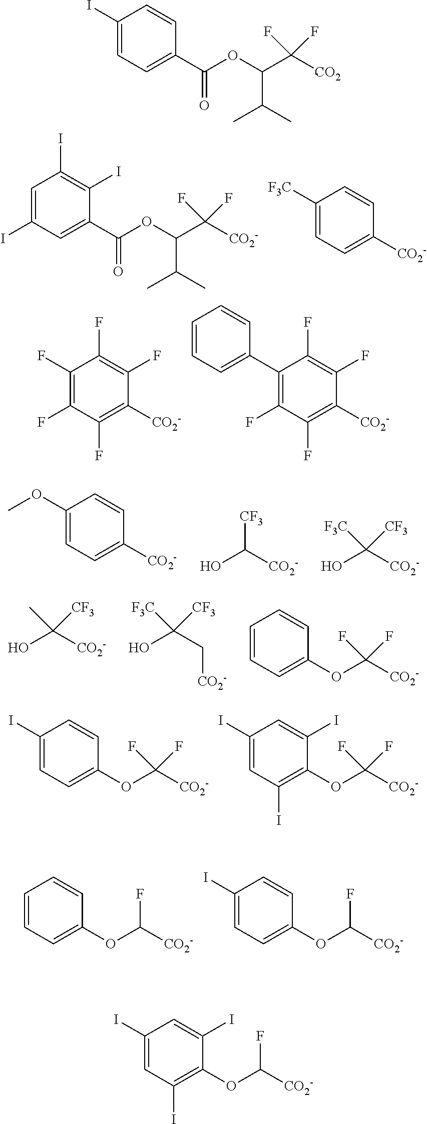

In formulae (1) and (2), Mq⁺ is an onium cation. The onium cation is preferably a sulfonium, iodonium or ammonium cation. Examples of the sulfonium cation include structures as exemplified for the sulfonium cation in the sulfonium salt monomer of formula (a), structures as described in JP-A 2024-3744, paragraphs [0102]-[0125] and JP-A 2023-169812, paragraphs [0070]-[0085], and structures of formula (d3), but not limited thereto. Examples of the iodonium cation are as described in JP-A 2024-000259, paragraph [0181], but not limited thereto. Preferably, the ammonium cation has the formula (am-1).

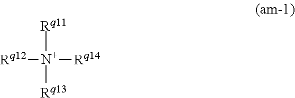

In formula (am-1), R^{q11} to R^{g14} are each independently a C₁-C₄₀ hydrocarbyl group which may contain a heteroatom. R^{ct6} and R^{ct7} may bond together to form a ring with the nitrogen atom to which they are attached. Examples of the hydrocarbyl group are as exemplified above as the hydrocarbyl group R² in formula (a).

Examples of the ammonium cation represented by formula (am-1) are shown below, but not limited thereto.

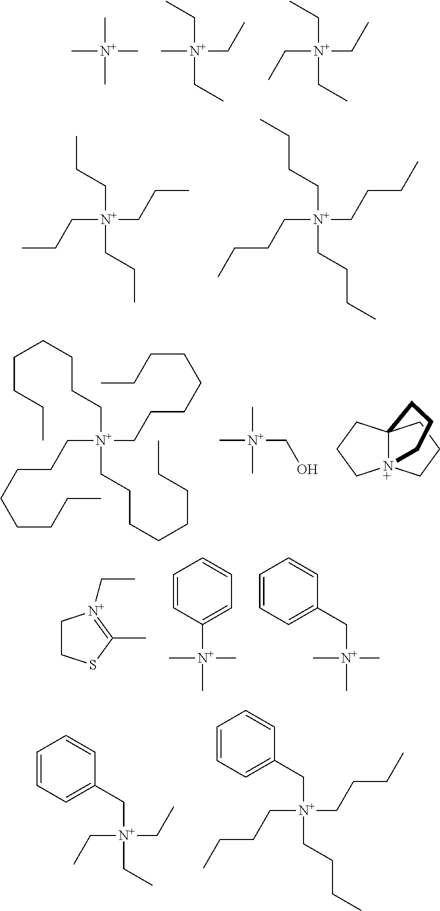

Examples of the onium salt having formula (1) or (2) include arbitrary combinations of anions with cations, both as exemplified above. These onium salts may be readily prepared by ion exchange reaction using any well-known organic chemistry technique. For the ion exchange reaction, reference may be made to JP-A 2007-145797, for example.

The onium salt having formula (1) or (2) functions as a quencher in the chemically amplified resist composition because the counter anion of the onium salt is a conjugated base of a weak acid. This is because the counter anion of the onium salt is a conjugated base of a weak acid. As used herein, the weak acid indicates an acidity insufficient to deprotect an acid labile group from an acid labile group-containing unit for the base polymer. The onium salt having formula (1) or (2) functions as a quencher when used in combination with an onium salt type photoacid generator having a conjugated base of a strong acid (typically a sulfonic acid which is fluorinated at α-position) as the counter anion. In a system using a mixture of an onium salt capable of generating a strong acid (e.g., α-position fluorinated sulfonic acid) and an onium salt capable of generating a weak acid (e.g., non-fluorinated sulfonic acid or carboxylic acid), if the strong acid generated from the photoacid generator upon exposure to high-energy radiation collides with the unreacted onium salt having a weak acid anion, then a salt exchange occurs whereby the weak acid is released and an onium salt having a strong acid anion is formed. In this course, the strong acid is exchanged into an acid having a low catalysis, incurring apparent deactivation of the acid for enabling to control acid diffusion.

JP 6848776 discloses an onium salt having sulfonium cation and phenoxide anion sites in the same molecule, JP 6583136 and JP-A 2020-200311 disclose an onium salt having sulfonium cation and carboxylate anion sites in the same molecule, and JP 6274755 discloses an onium salt having iodonium cation and carboxylate anion sites in the same molecule. These onium salts may also be used as the quencher (C).

If a photoacid generator capable of generating a strong acid is an onium salt, an exchange from the strong acid generated upon exposure to high-energy radiation to a weak acid as above can take place, but it rarely happens that the weak acid generated upon exposure to high-energy radiation collides with the unreacted onium salt capable of generating a strong acid to induce a salt exchange. This is because of a likelihood of an onium cation forming an ion pair with a stronger acid anion.

When the inventive chemically amplified resist composition comprises an onium salt of formula (1) or (2) as the quencher (C), the amount of the onium salt used is preferably 0.1 to 20 parts by weight, more preferably 0.1 to 10 parts by weight per 80 parts by weight of the base polymer (A). As long as the content of onium salt type quencher (C) is in the range, a satisfactory resolution is available without a substantial lowering of sensitivity. The onium salt having formula (1) or (2) may be used alone or in admixture.

The inventive chemically amplified resist composition may comprise a nitrogen-containing compound as the quencher (C). Examples of the nitrogen-containing compound (C) include primary, secondary and tertiary amine compounds, specifically amine compounds having a hydroxy group, ether bond, ester bond, lactone ring, cyano group or sulfonic ester bond, as described in JP A 2008-111103, paragraphs [0146]-[0164], and primary or secondary amine compounds protected with a carbamate group, as described in JP 3790649.

A sulfonic acid sulfonium salt having a nitrogen-containing substituent may also be used as the nitrogen-containing compound. This compound functions as a quencher in the unexposed region, but as a so-called photo-degradable base in the exposed region because it loses the quencher function in the exposed region due to neutralization thereof with the acid generated by itself. Using a photo-degradable base, the contrast between exposed and unexposed regions can be further enhanced. With respect to the photo-degradable base, reference may be made to JP-A 2009-109595 and JP-A 2012-46501, for example.

When the inventive chemically amplified resist composition comprises a nitrogen-containing compound as the quencher (C), the amount of the nitrogen-containing compound used is preferably 0.001 to 12 parts by weight, more preferably 0.01 to 8 parts by weight per 80 parts by weight of the base polymer (A). The nitrogen-containing compound may be used alone or in admixture.

### (D) Acid Generator

The inventive chemically amplified resist composition may comprise an acid generator as long as the benefits of the present invention is not compromised. The acid generator is typically a compound (photoacid generator) capable of generating an acid upon exposure to actinic ray or radiation. Although the photoacid generator used herein is not particularly limited as long as it is capable of generating an acid upon exposure to high-energy radiation, those compounds capable of generating sulfonic acid, imide acid (imidic acid) or methide acid are preferred. Suitable photoacid generators include sulfonium salts, iodonium salts, sulfonyldiazomethane, N-sulfonyloxyimide, and oxime-O-sulfonate acid generators. Exemplary acid generators are described in JP-A 2008-111103, paragraphs [0122]-[0142].

Also, a sulfonium salt having the formula (3-1) and an iodonium salt having the formula (3-2) are advantageously used as the other photoacid generator.

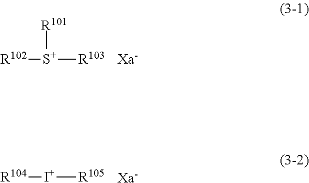

In formulae (3-1) and (3-2), R¹⁰¹ to R¹⁰⁵ are each independently halogen, or a C₁-C₂₀ hydrocarbyl group which may contain a heteroatom. Examples of the halogen and hydrocarbyl group are as exemplified above as the halogen and hydrocarbyl group R² in formula (a). In the hydrocarbyl group, some or all hydrogen atoms may be substituted by a moiety containing a heteroatom such as oxygen, sulfur, nitrogen or halogen and some constituent —CH₂— may be replaced by a moiety containing a heteroatom such as oxygen, sulfur or nitrogen, so that the group may contain hydroxy, fluorine, chlorine, bromine, iodine, cyano, nitro, carbonyl, ether bond, ester bond, sulfonic ester bond, carbonate bond, lactone ring, sultone ring, carboxylic anhydride (—C(═O)—O—C(═O)—), or haloalkyl moiety. R¹⁰¹ and R¹⁰² may bond together to form a ring with a sulfur atom to which they are attached. Examples of the ring formed here are as exemplified for formula (a) where two of three substituents bonded to S⁺ bond together to form a ring with the sulfur atom to which they are attached.

Examples of the cation in the sulfonium salt having formula (3-1) include structures as exemplified for the sulfonium cation in the sulfonium salt monomer of formula (a), structures as described in JP-A 2024-3744, paragraphs [0102]-[0125] and JP-A 2023-169812, paragraphs [0070]-[0085], and structures of formula (d3), but not limited thereto. Examples of the cation in the iodonium salt having formula (3-2) are as described in JP-A 2024-000259, paragraph [0181], but not limited thereto.

In formulae (3-1) and (3-2), Xa is an anion of the following formula (3A), (3B), (3C) or (3D).

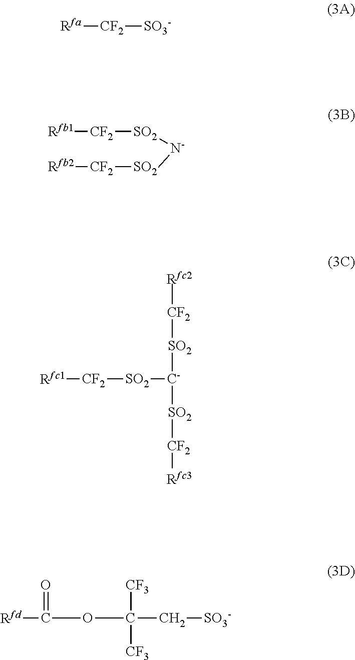

In formula (3A), R^{fa} is fluorine, or a C₁-C₄₀ hydrocarbyl group which may contain a heteroatom. The hydrocarbyl group may be saturated or unsaturated, and may be straight, branched, or cyclic. Examples thereof are as will be exemplified later for hydrocarbyl group R^{fa1} in formula (3A′).

Of the anions of formula (3A), a structure having formula (3A′) is preferred.

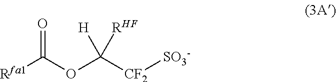

In formula (3A′), R^{HF} is hydrogen or a trifluoromethyl group, preferably a trifluoromethyl group.

In formula (3A′), R^{fa1} is a C₁-C₃₈ hydrocarbyl group which may contain a heteroatom. Suitable heteroatoms include oxygen, nitrogen, sulfur and halogen, with oxygen being preferred. Of the hydrocarbyl groups, those of 6 to 30 carbon atoms are preferred because a high resolution is available in fine pattern formation.

The C₁-C₃₈ hydrocarbyl group R¹¹ may be saturated or unsaturated and straight, branched or cyclic. Examples thereof include C₁-C₃₈ alkyl groups such as methyl, ethyl, n-propyl, isopropyl, n-butyl, isobutyl, sec-butyl, tert-butyl, pentyl, neopentyl, hexyl, heptyl, 2-ethylhexyl, nonyl, undecyl, tridecyl, pentadecyl, heptadecyl and icocyl groups; C₃-C₃₈ cyclic saturated hydrocarbyl groups such as cyclopentyl, cyclohexyl, 1-adamantyl, 2-adamantyl, 1-adamantylmethyl, norbornyl, norbornylmethyl, tricyclodecyl, tetracyclodecyl, tetracyclodecylmethyl and dicyclohexylmethyl groups; C₂-C₃₈ unsaturated aliphatic hydrocarbyl groups such as allyl and 3-cyclohexenyl groups; C₆-C₃₈ aryl groups such as phenyl, 1-naphthyl and 2-naphthyl groups; C₇-C₃₈ aralkyl groups such as benzyl and diphenylmethyl groups; and combinations thereof.

Some or all of hydrogen atoms of the hydrocarbyl group may be replaced by a group containing a heteroatom such as oxygen, sulfur, nitrogen or halogen, some constituent —CH₂— of the hydrocarbyl group may be replaced by a group containing a heteroatom such as oxygen, sulfur or nitrogen, and as a result, the hydrocarbyl group may contain a hydroxy group, fluorine, chlorine, bromine, iodine, a cyano group, a carbonyl group, an ether bond, an ester bond, a sulfonic ester bond, a carbonate bond, a lactone ring, a sultone ring, carboxylic anhydride (—C(═O)—O—C(═O)—), a haloalkyl group, or the like.

The heteroatom is preferably oxygen. Examples of the heteroatom-containing hydrocarbyl group include tetrahydrofuryl, methoxymethyl, ethoxymethyl, methylthiomethyl, acetamidomethyl, trifluoroethyl, (2-methoxyethoxy)methyl, acetoxymethyl, 2-carboxy-1-cyclohexyl, 2-oxopropyl, 4-oxo-1-adamantyl, and 3-oxocyclohexyl groups.

With respect to the synthesis of the sulfonium salt having an anion of formula (3A′), reference is made to JP-A 2007-145797, JP-A 2008-106045, JP-A 2009-7327, and JP-A 2009-258695. Also useful are the sulfonium salts described in JP-A 2010-215608, JP-A 2012-41320, JP-A 2012-106986, and JP-A 2012-153644.

Examples of the anion having formula (3A) are shown below, but not limited thereto. In the following formulae, Ac is an acetyl group.

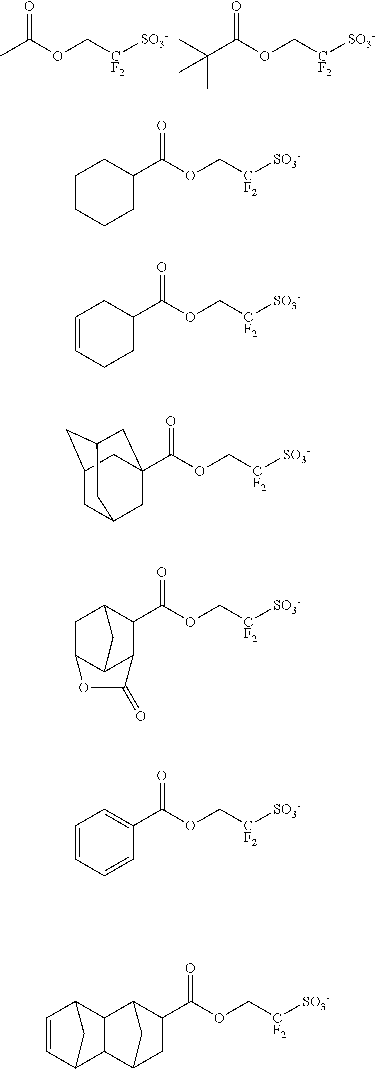

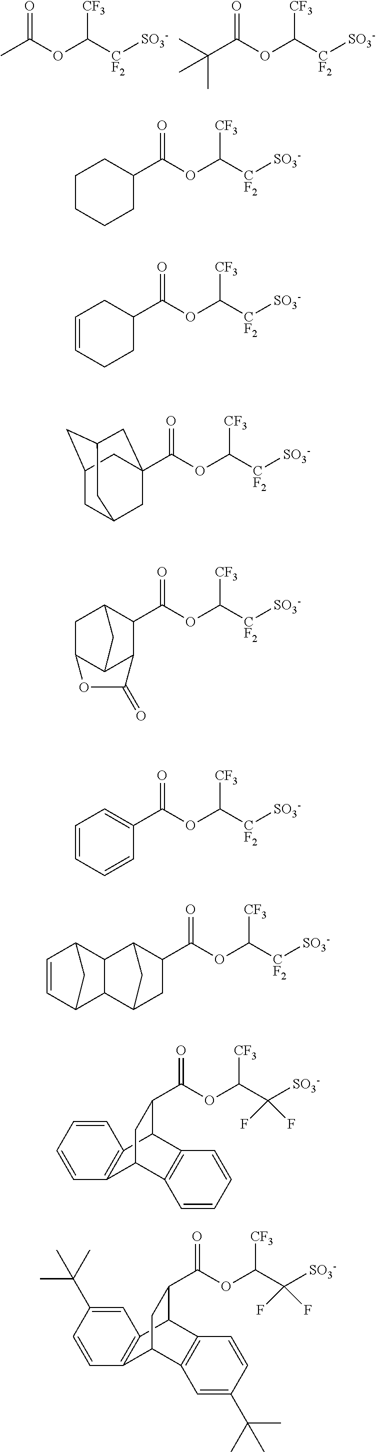

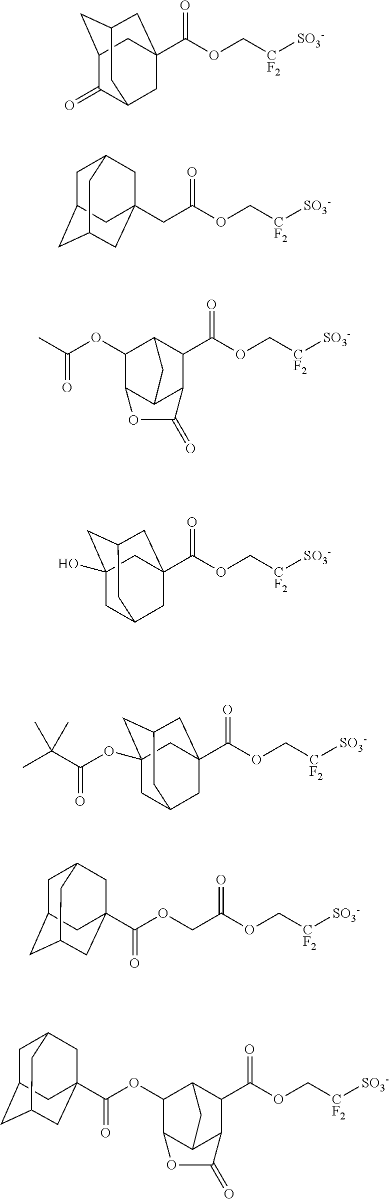

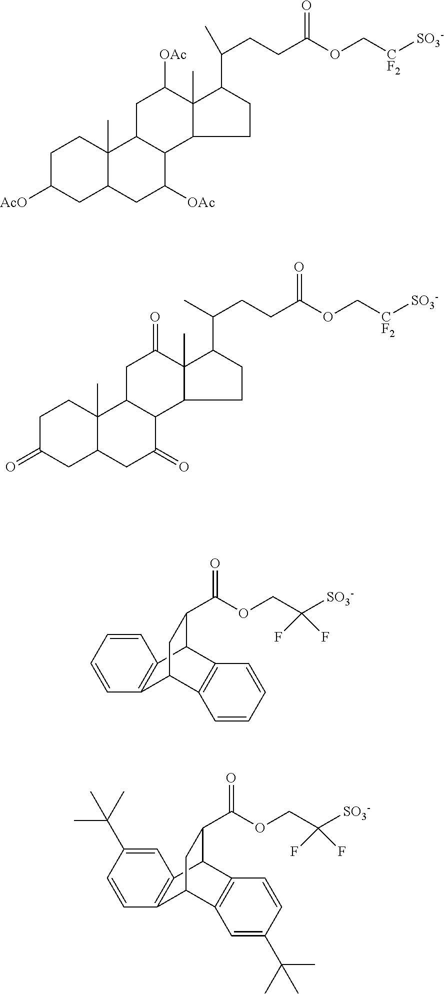

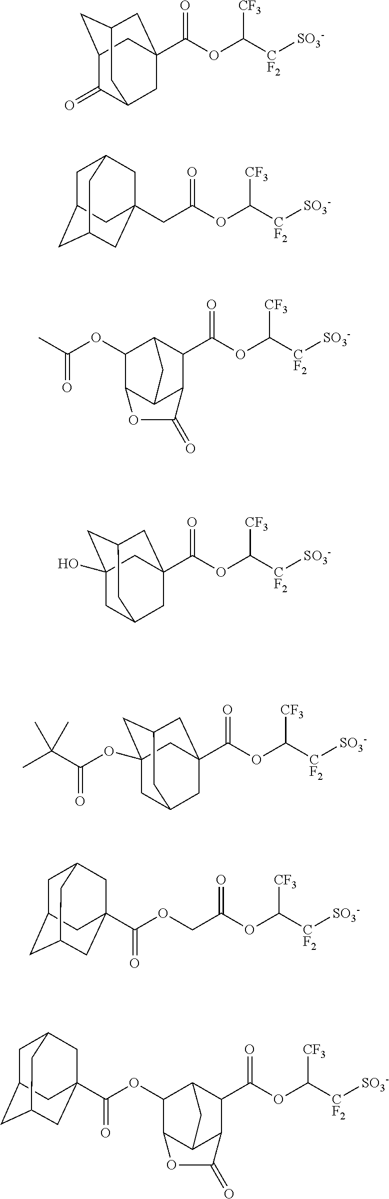

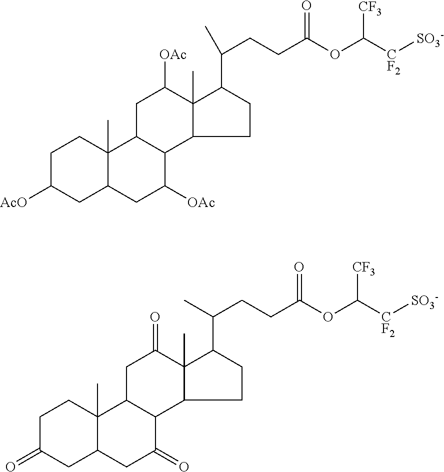

In formula (3B), R^{b1} and R^{b2} are each independently fluorine, or a C₁-C₄₀ hydrocarbyl group which may contain a heteroatom. The hydrocarbyl group may be saturated or unsaturated, and may be straight, branched, or cyclic. Examples of the hydrocarbyl group are as exemplified above as a hydrocarbyl group R^{fa1} in formula (3A′). Preferably R^{fb1} and R^{fb2} each are fluorine or a straight C₁-C₄ fluorinated alkyl group. A pair of R^{fb1} and R^{fb2} may bond together to form a ring with the linkage (—CF₂—SO₂—N—SO₂—CF₂—) to which they are attached, and the R^{fb1}-R^{fb2} group is preferably a fluorinated ethylene or fluorinated propylene group.

In formula (3C), R^{fc1}, R^{fc2} and R^{fc3} are each independently fluorine, or a C₁-C₄₀ hydrocarbyl group which may contain a heteroatom. The hydrocarbyl group may be saturated or unsaturated, and may be straight, branched, or cyclic. Examples of the hydrocarbyl group are as exemplified above as a hydrocarbyl group R^{fa1} in formula (3A′). Preferably R^{fc1}, R^{fc2} and R^{fc3} each are fluorine or a straight C₁-C₄ fluorinated alkyl group. A pair of R^{fc1} and R^{fc2} may bond together to form a ring with the linkage (—CF₂—SO₂—C⁻—SO₂—CF₂—) to which they are attached, and the R^{fc1}-R^{fc2} group is preferably a fluorinated ethylene or fluorinated propylene group.

In formula (3D), R^{fd} is a C₁-C₄₀ hydrocarbyl group which may contain a heteroatom. The hydrocarbyl group may be saturated or unsaturated, and may be straight, branched, or cyclic. Examples of the hydrocarbyl group are as exemplified above as a hydrocarbyl group R^{fa1} in formula (3A′).

With respect to the synthesis of the sulfonium salt having an anion of formula (3D), reference is made to JP-A 2010-215608, and JP-A 2014-133723.

Examples of the anion having formula (3D) are shown below, but not limited thereto.

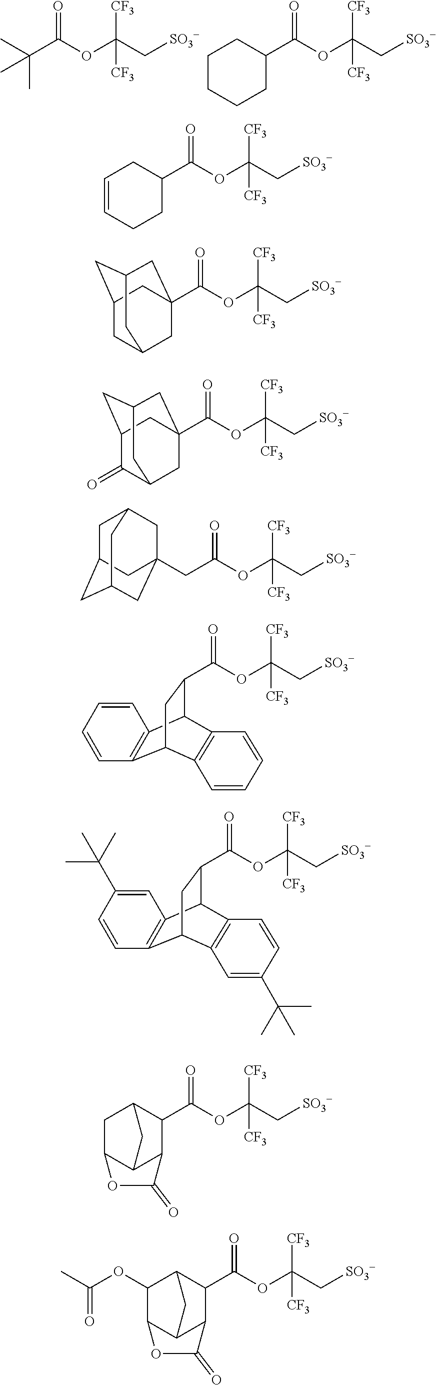

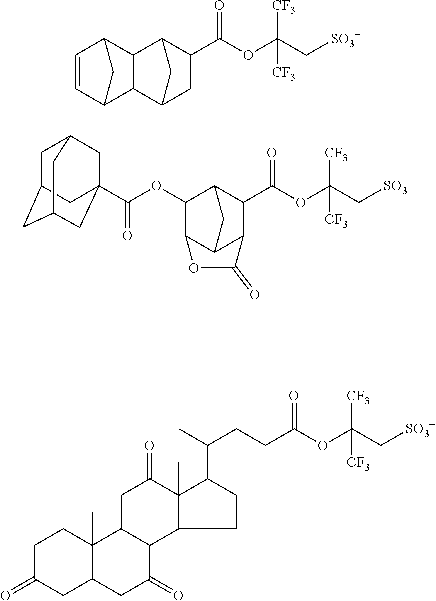

The photoacid generator having the anion of formula (3D) does not have fluorine at the α-position relative to the sulfo group, but two trifluoromethyl groups at the β-position. For this reason, it has a sufficient acidity to sever the acid labile groups in the base polymer. Thus the compound is an effective photoacid generator.

Also, a compound having the formula (4) can be suitably used as the photoacid generator.

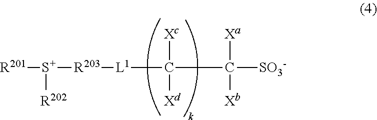

In formula (4), R²⁰¹ to R²⁰² are each independently a C₁-C₃₀ hydrocarbyl group which may contain a heteroatom. R²⁰³ is a C₁-C₃₀ hydrocarbylene group which may contain a heteroatom. Any two of R²⁰¹ and R²⁰² and R²⁰³ may bond together to form a ring with a sulfur atom to which they are attached. Here, examples of the ring formed here are as exemplified for formula (a) where two of three substituents bonded to S⁺ bond together to form a ring with the sulfur atom to which they are attached.

The C₁-C₃₀ hydrocarbyl groups R²⁰¹ and R²⁰² may be saturated or unsaturated and straight, branched or cyclic. Examples thereof include C₁-C₃₀ alkyl groups such as methyl, ethyl, n-propyl, isopropyl, n-butyl, isobutyl, sec-butyl, tert-butyl, n-pentyl, tert-pentyl, n-hexyl, n-octyl, 2-ethylhexyl, n-nonyl and n-decyl groups; C₃-C₃₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, oxanorbornyl, tricyclo[5.2.1.0^{2,6}]decyl and adamantyl groups; C₆-C₃₀ aryl groups such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl, tert-butylnaphthyl and anthracenyl groups; and combinations thereof. Some or all of hydrogen atoms of the hydrocarbyl group may be replaced by a group containing a heteroatom such as oxygen, sulfur, nitrogen or halogen, some constituent —CH₂— of the hydrocarbyl group may be replaced by a group containing a heteroatom such as oxygen, sulfur or nitrogen, and as a result, the hydrocarbyl group may contain a hydroxy group, a cyano group, fluorine, chlorine, bromine, iodine, a carbonyl group, an ether bond, an ester bond, a sulfonic ester bond, a carbonate bond, a lactone ring, a sultone ring, carboxylic anhydride (—C(═O)—O—C(═O)—), a haloalkyl group, or the like.

The C₁-C₃₀ hydrocarbylene group R²⁰³ may be saturated or unsaturated and straight, branched or cyclic. Examples thereof include C₁-C₃₀ alkanediyl groups such as methanediyl, ethane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1, 14-diyl, pentadecane-1, 15-diyl, hexadecane-1,16-diyl and heptadecane-1,17-diyl groups; C₃-C₃₀ cyclic saturated hydrocarbylene groups such as cyclopentanediyl, cyclohexanediyl, norbornanediyl and adamantanediyl groups; and C₆-C₃₀ arylene groups such as phenylene, methylphenylene, ethylphenylene, n-propylphenylene, isopropylphenylene, n-butylphenylene, isobutylphenylene, sec-butylphenylene, tert-butylphenylene, naphthylene, methylnaphthylene, ethylnaphthylene, n-propylnaphthylene, isopropylnaphthylene, n-butylnaphthylene, isobutylnaphthylene, sec-butylnaphthylene and tert-butylnaphthylene groups; and combinations thereof. Some or all of hydrogen atoms of the hydrocarbylene group may be replaced by a group containing a heteroatom such as oxygen, sulfur, nitrogen or halogen, some constituent —CH₂— of the hydrocarbylene group may be replaced by a group containing a heteroatom such as oxygen, sulfur or nitrogen, and as a result, the hydrocarbylene group may contain a hydroxy group, a cyano group, fluorine, chlorine, bromine, iodine, a carbonyl group, an ether bond, an ester bond, a sulfonic ester bond, a carbonate bond, a lactone ring, a sultone ring, carboxylic anhydride (—C(═O)—O—C(═O)—), a haloalkyl group, or the like. The heteroatom is preferably oxygen.

In formula (4), L¹ is a single bond, an ether bond, or a C₁-C₂₀ hydrocarbylene group which may contain a heteroatom. The hydrocarbylene group may be saturated or unsaturated and straight, branched or cyclic. Examples of the hydrocarbylene group are as exemplified above as a hydrocarbylene group R²⁰³. In formula (4), X^{a}, X^{b}, X^{c} and X^{d} are each independently hydrogen, fluorine or a trifluoromethyl group. It is to be noted that at least one of X^{a}, X^{b}, X^{c} and X^{d} is fluorine or a trifluoromethyl group.

In formula (4), k is 0, 1, 2 or 3.

Preferably, the photoacid generator of formula (4) has the following formula (4′).

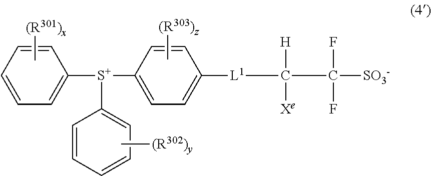

In formula (4′), L¹ is as defined above. X^{e} is hydrogen or a trifluoromethyl group, preferably a trifluoromethyl group. R³⁰¹, R³⁰² and R³⁰³ each independently a C₁-C₂₀ hydrocarbyl group which may contain a heteroatom. The hydrocarbyl group may be saturated or unsaturated, and may be straight, branched, or cyclic. Examples of the hydrocarbyl group are as exemplified above as a hydrocarbyl group R^{fa1} in formula (3A′). The subscripts x and y are each independently 0, 1, 2, 3, 4 or 5. The subscript z is 0, 1, 2, 3 or 4.

Examples of the photoacid generator having formula (4) are as exemplified as for the photoacid generator having formula (2) in JP-A 2017-26980.

Of the foregoing photoacid generators, those having an anion of formula (3A′) or (3D) are especially preferred because of reduced acid diffusion and high solubility in the resist solvent. Also those having formula (4′) are especially preferred because of extremely reduced acid diffusion.

As other acid generators, sulfonium salts and iodonium salts having the following formula (5-1) or (5-2) may also be used. These salts contain an anion having an aromatic ring substituted with iodine.

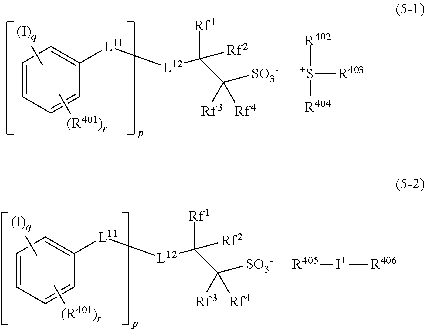

In formulae (5-1) and (5-2), p is 1, 2 or 3. The subscript q is 0, 1, 2, 3, 4 or 5. The subscript r is 0, 1, 2 or 3. The sum of q+r is 1 or more and 5 or less. The subscript q is preferably 1, 2 or 3, more preferably 2 or 3. The subscript r is preferably 0, 1 or 2.

In formulae (5-1) and (5-2), L¹¹ is a single bond, an ether bond, an ester bond, or a C₁-C₆ saturated hydrocarbylene group which may contain an ether bond or an ester bond. The saturated hydrocarbylene group may be straight, branched or cyclic.

In formulae (5-1) and (5-2), L¹² is a single bond or a C₁-C₂₀ divalent linking group when p=1, and a C₁-C₂₀ (p+1)-valent linking group which may contain oxygen atom, sulfur atom or nitrogen atom when p=2 or 3.

In formulae (5-1) and (5-2), R⁴⁰¹ is a hydroxy group, a carboxy group, fluorine, chlorine, bromine, or an amino group, or a C₁-C₂₀ hydrocarbyl group, C₁-C₂₀ hydrocarbyloxy group, C₂-C₂₀ hydrocarbylcarbonyl group, C₂-C₂₀ hydrocarbyloxycarbonyl group, C₂-C₂₀ hydrocarbylcarbonyloxy group, or C₁-C₂₀ hydrocarbylsulfonyloxy group, which may contain fluorine, chlorine, bromine, a hydroxy group, an amino group or an ether bond, or —N(R^{401A})(R^{401B})—N(R^{401C})—C(═O)—R^{401D} or —N(R^{401C})—C(═O)—O—R^{401D}. R^{401A} and R^{401B} are each independently hydrogen, or a C₁-C₆ saturated hydrocarbyl group. R^{401C} is hydrogen, or a C₁-C₆ saturated hydrocarbyl group which may contain halogen, a hydroxy group, a C₁-C₆ saturated hydrocarbyloxy group, a C₂-C₆ saturated hydrocarbylcarbonyl group, or a C₂-C₆ saturated hydrocarbylcarbonyloxy group. R^{401D} is a C₁-C₁₆ aliphatic hydrocarbyl group, a C₆-C₁₄ aryl group, or a C₇-C₁₅ aralkyl group, halogen, a hydroxy group, a C₁-C₆ saturated hydrocarbyloxy group, a C₂-C₆ saturated hydrocarbylcarbonyl group, or a C₂-C₆ saturated hydrocarbylcarbonyloxy group. The aliphatic hydrocarbyl group may be saturated or unsaturated and straight, branched or cyclic. The hydrocarbyl, hydrocarbyloxy, hydrocarbylcarbonyl, hydrocarbyloxycarbonyl, hydrocarbylcarbonyloxy and hydrocarbylsulfonyloxy groups may be straight, branched or cyclic. A plurality of R⁴⁰¹ may be identical or different when p and/or r is 2 or more.

Of these, R⁴⁰¹ is preferably hydroxy group, —N(R^{401C})—C(═O)—R^{401D}, —N(R^{401C})—C(═O)—O—R^{401D}, fluorine, chlorine, bromine, a methyl group, or a methoxy group.

In formula (5-1) and (5-2), Rf¹ to Rf⁴ are each independently hydrogen, fluorine or a trifluoromethyl group, and at least one of Rf¹ to Rf⁴ is fluorine or a trifluoromethyl group. Rf¹ and Rf², taken together, may form a carbonyl group. Particularly, both Rf³ and Rf⁴ are preferably fluorine.

In formulae (5-1) and (5-2), R⁴⁰² to R⁴⁰⁶ are each independently halogen, or a C₁-C₂₀ hydrocarbyl group which may contain a heteroatom. The hydrocarbyl group may be saturated or unsaturated, and may be straight, branched, or cyclic. Examples of thereof are as exemplified above as the hydrocarbyl group R² to in formula (a). Some or all of hydrogen atoms of the hydrocarbyl group may be replaced by a hydroxy group, a carboxy group, halogen, a cyano group, a nitro group, a mercapto group, a sultone ring, a sulfo group or a sulfonium salt-containing group, and some constituent —CH₂— of the hydrocarbyl group may be replaced by an ether bond, an ester bond, a carbonyl group, an amide bond, a carbonate bond, or a sulfonic ester bond. R⁴⁰² and R⁴⁰³ may bond together to form a ring with the sulfur atom to which they are attached. Here, examples of the ring formed here are as exemplified for formula (a) where two of three substituents bonded to S⁺ bond together to form a ring with the sulfur atom to which they are attached.

Examples of the cation in the sulfonium salt having formula (5-1) are as described in JP-A 2024-3744, paragraphs [0102]-[0125] and JP-A 2023-169812, paragraphs [0070]-[0085], and include cations of formula (d3). Examples of the cation in the iodonium salt having formula (5-2) are as described in JP-A 2024-000259, paragraph [0181].

Examples of the anion in the onium salt having formula (5-1) or (5-2) are shown below, but not limited thereto.

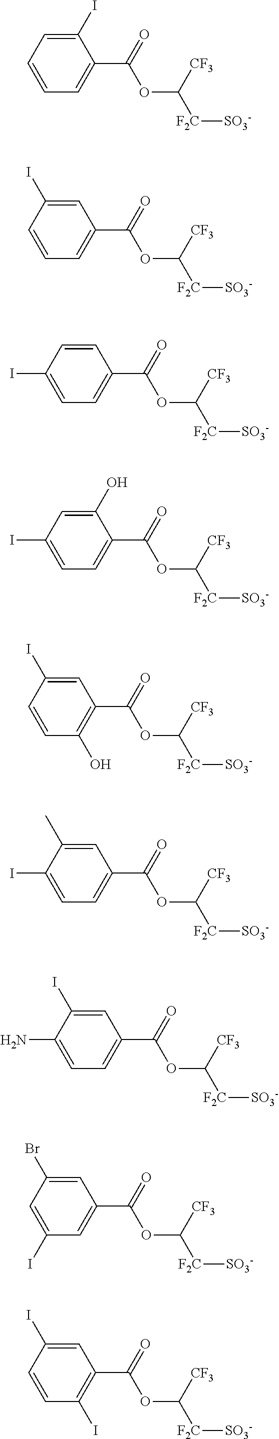

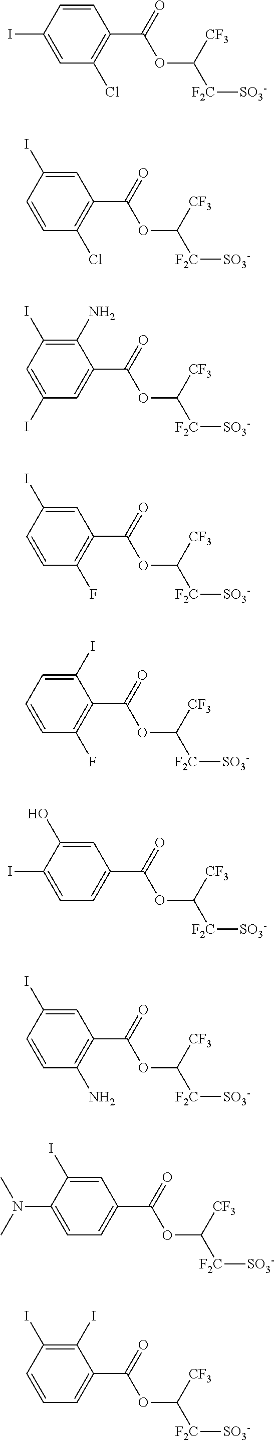

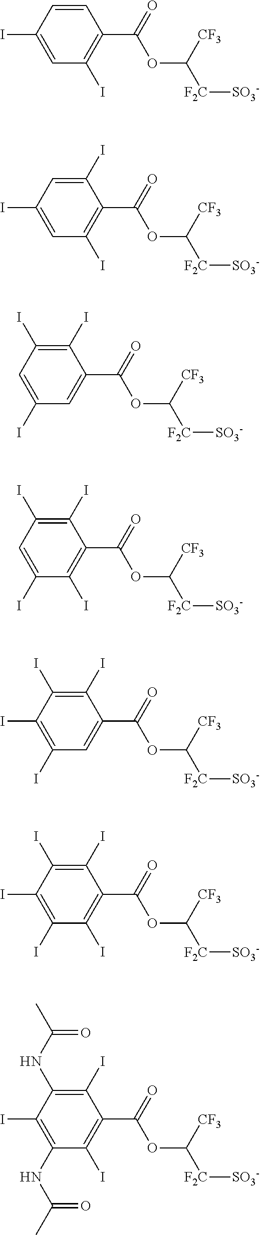

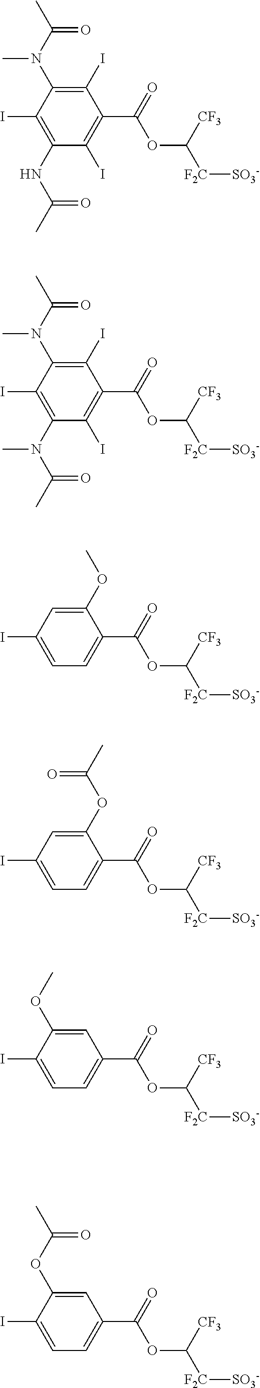

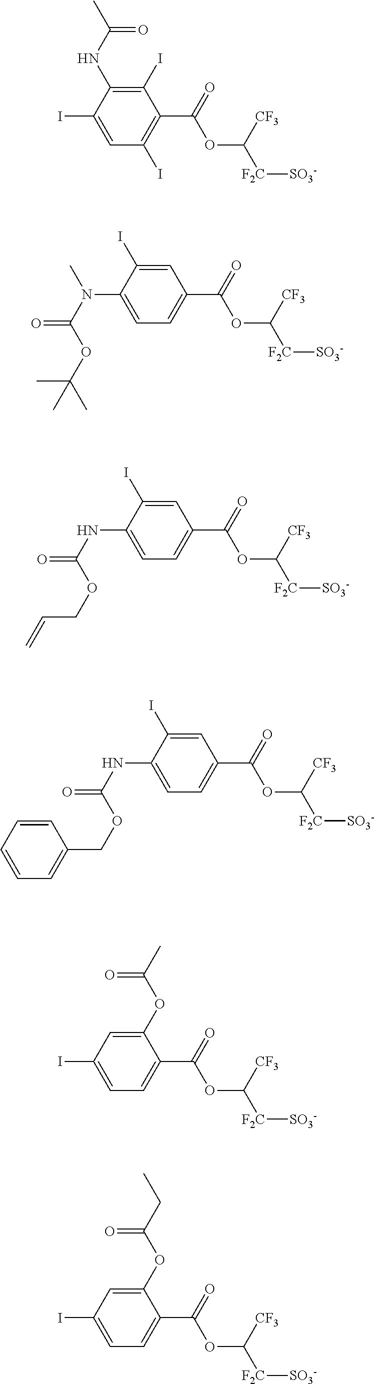

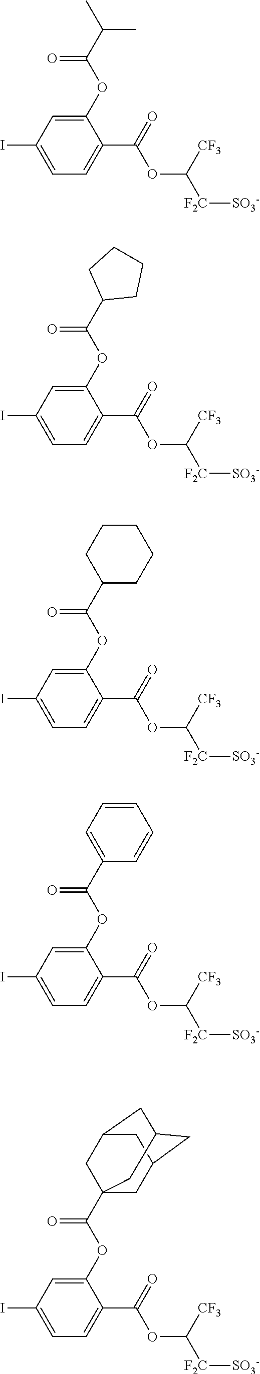

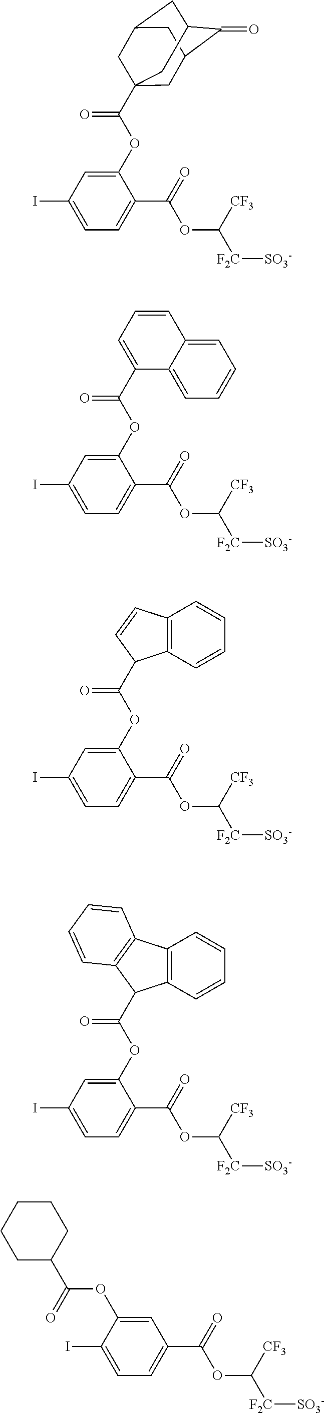

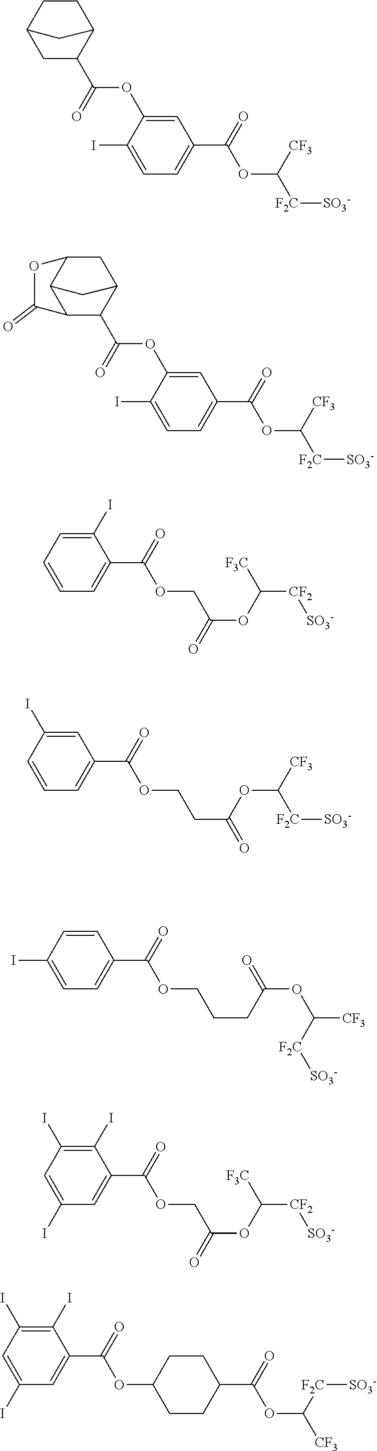

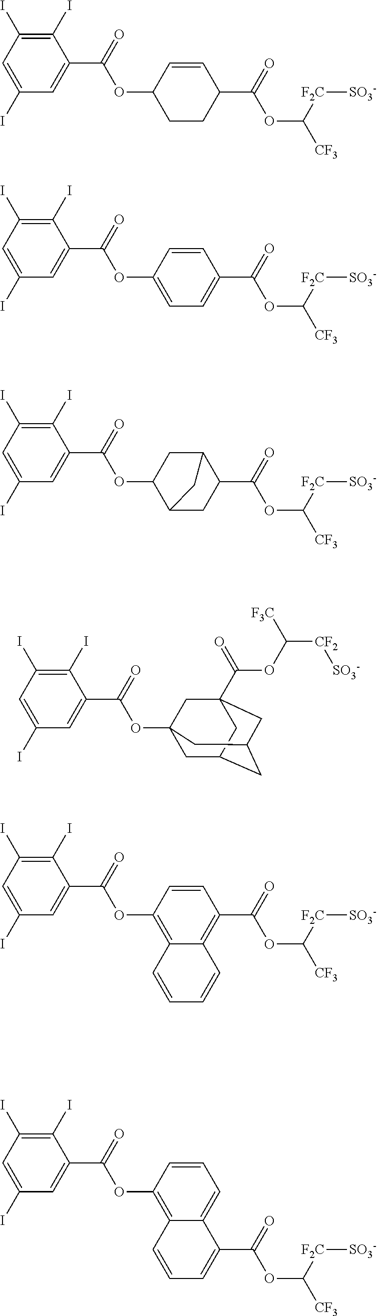

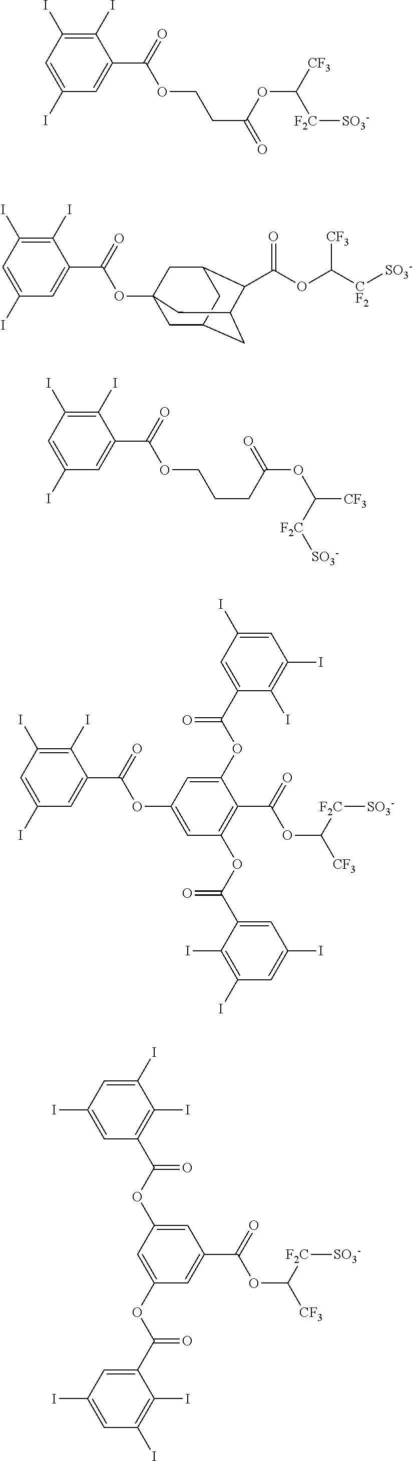

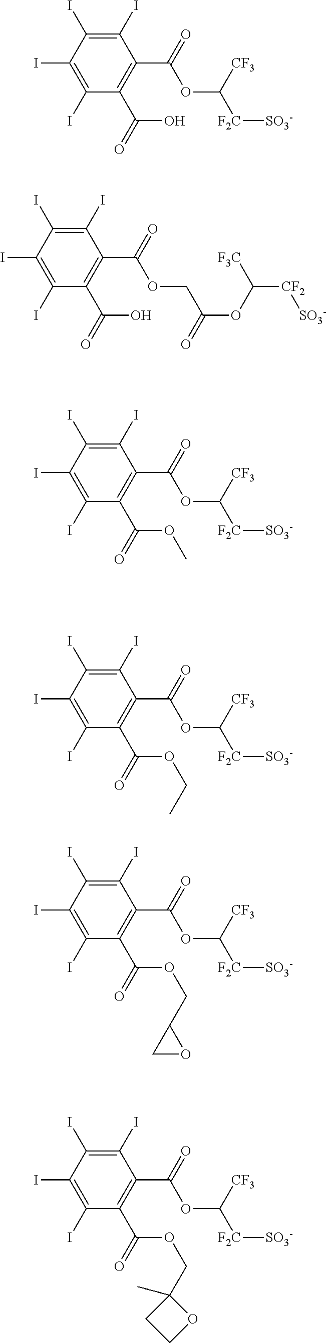

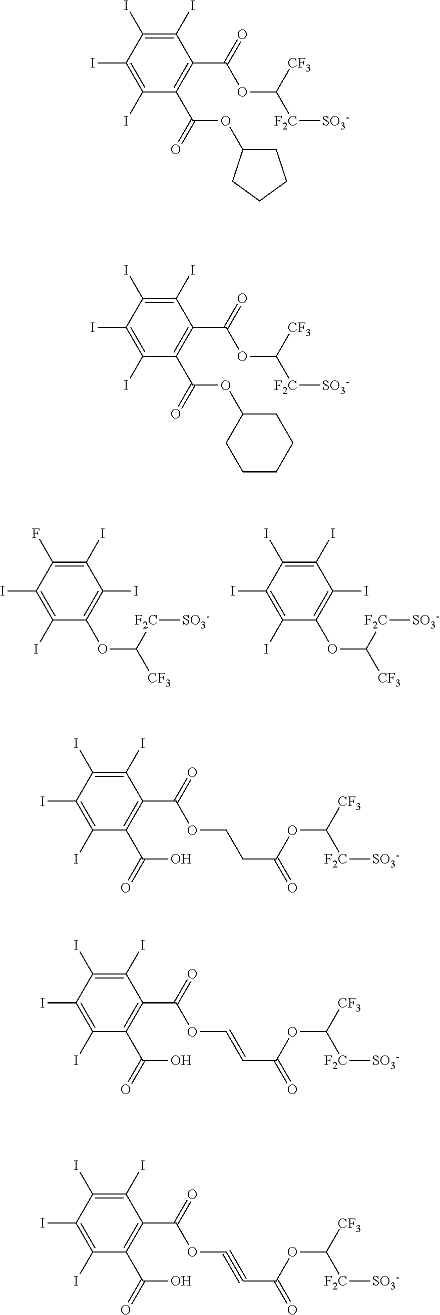

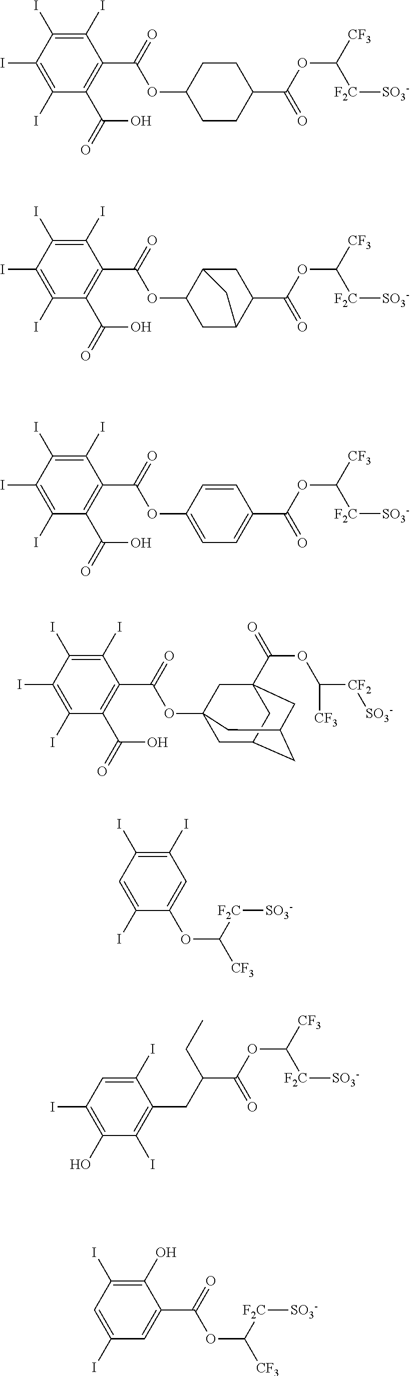

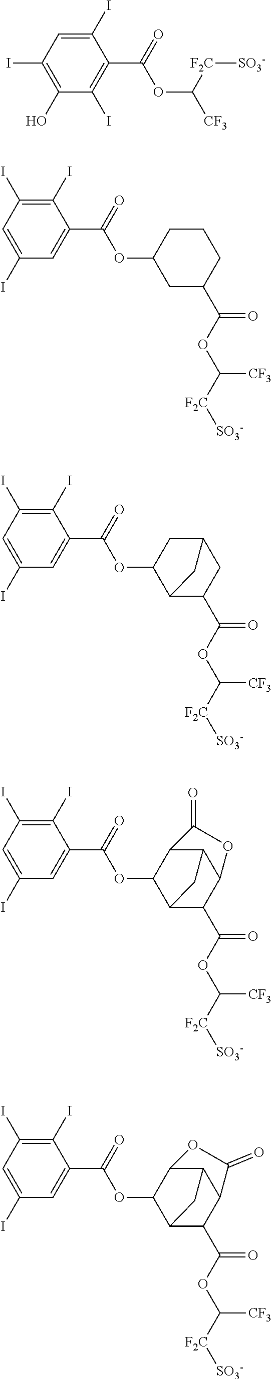

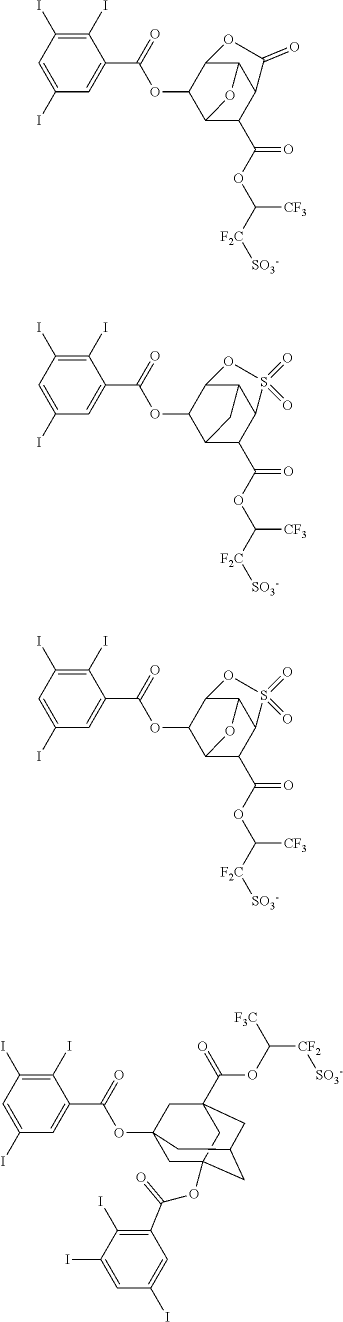

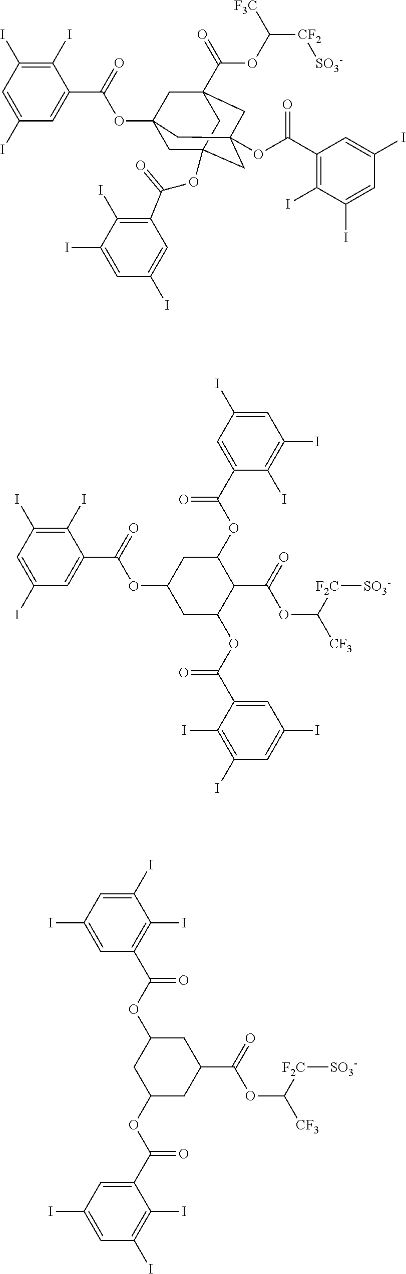

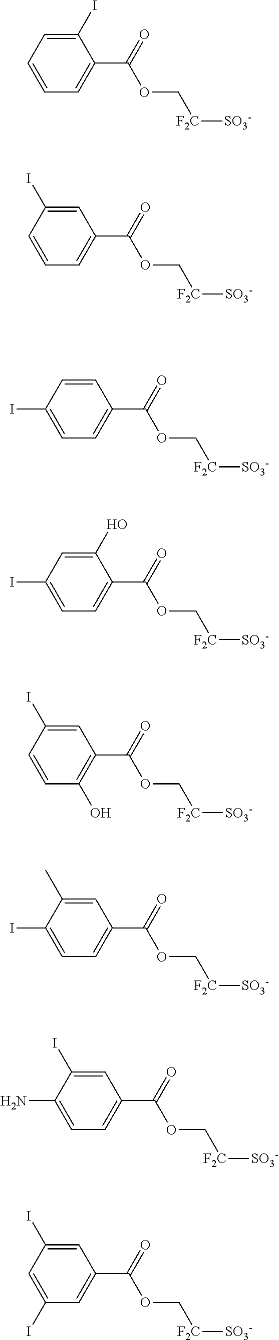

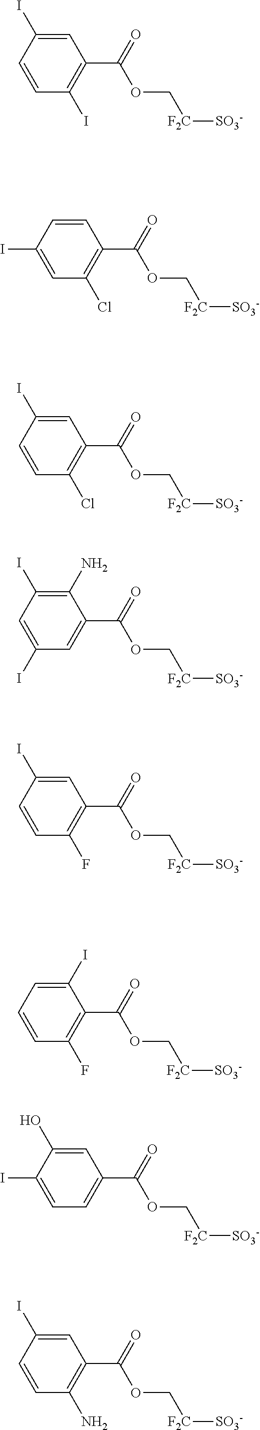

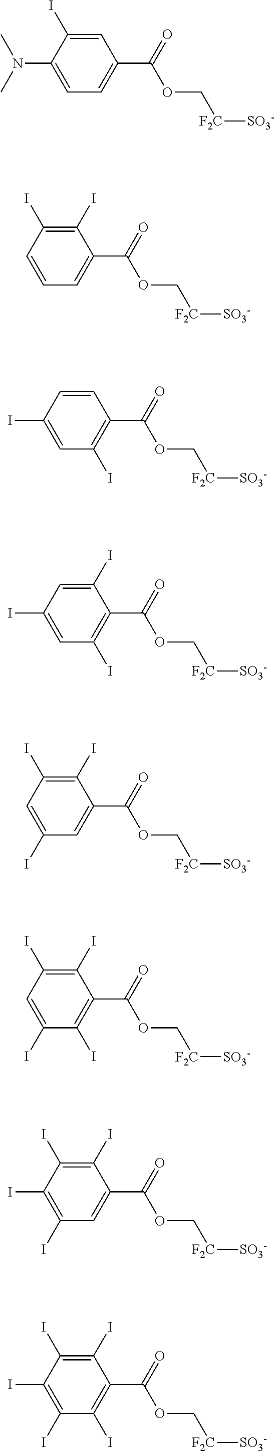

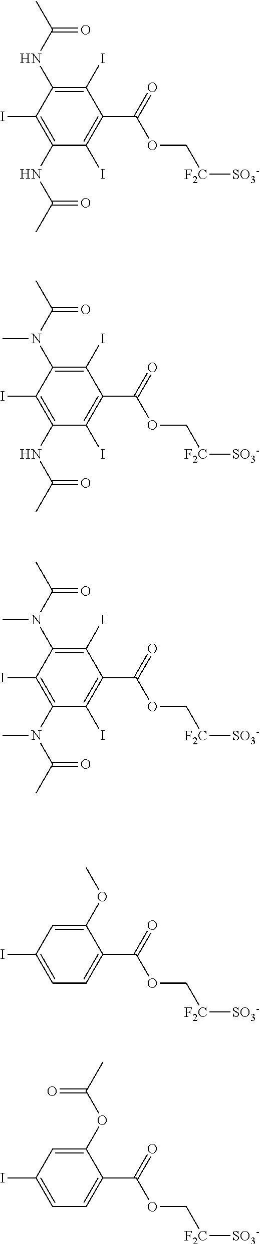

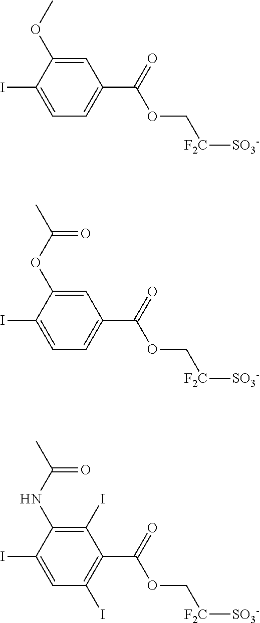

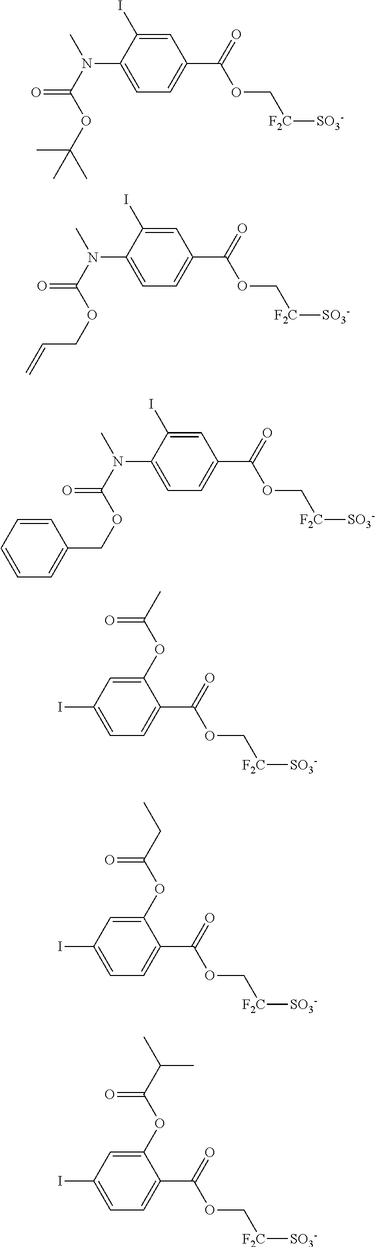

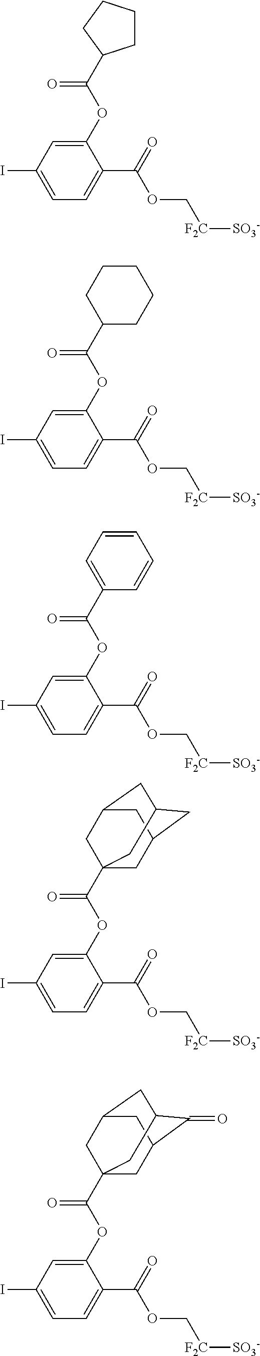

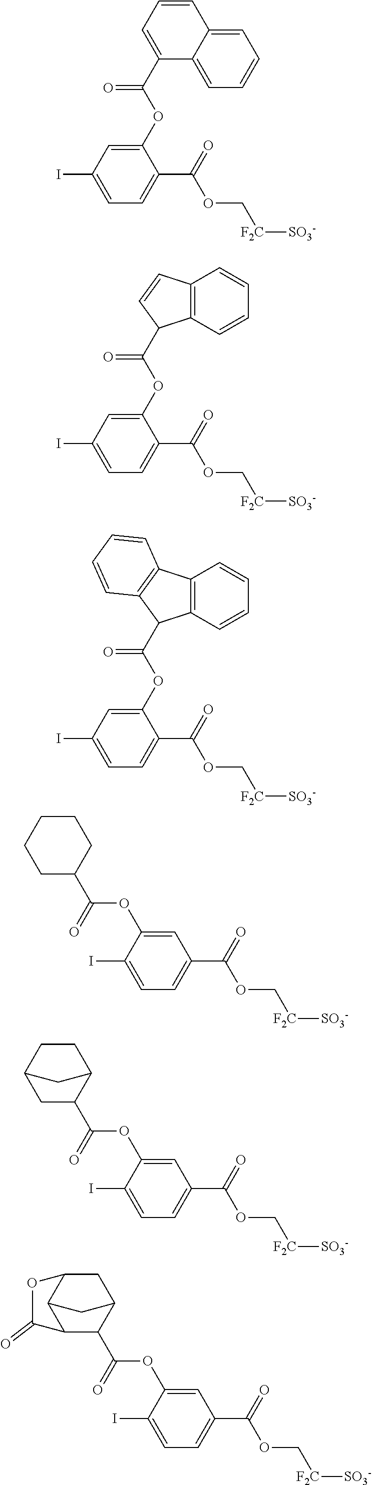

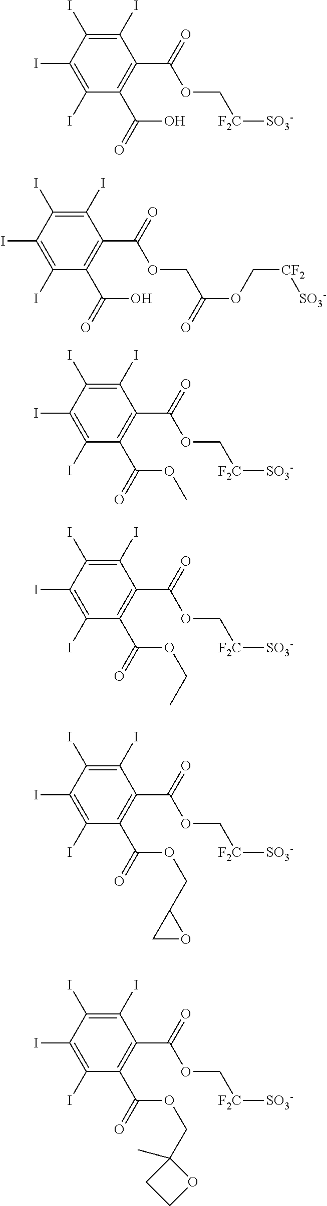

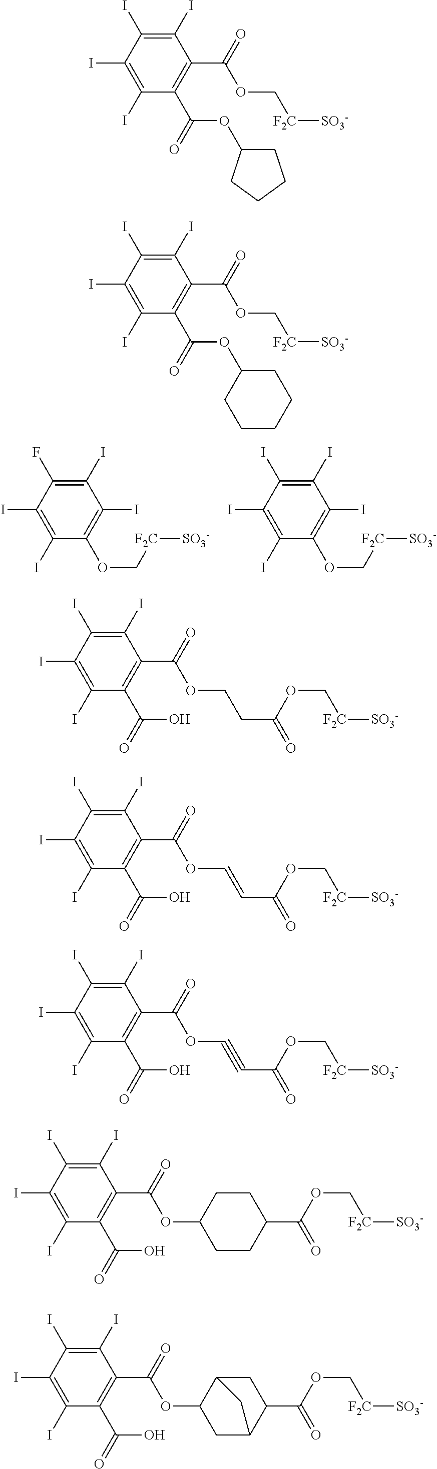

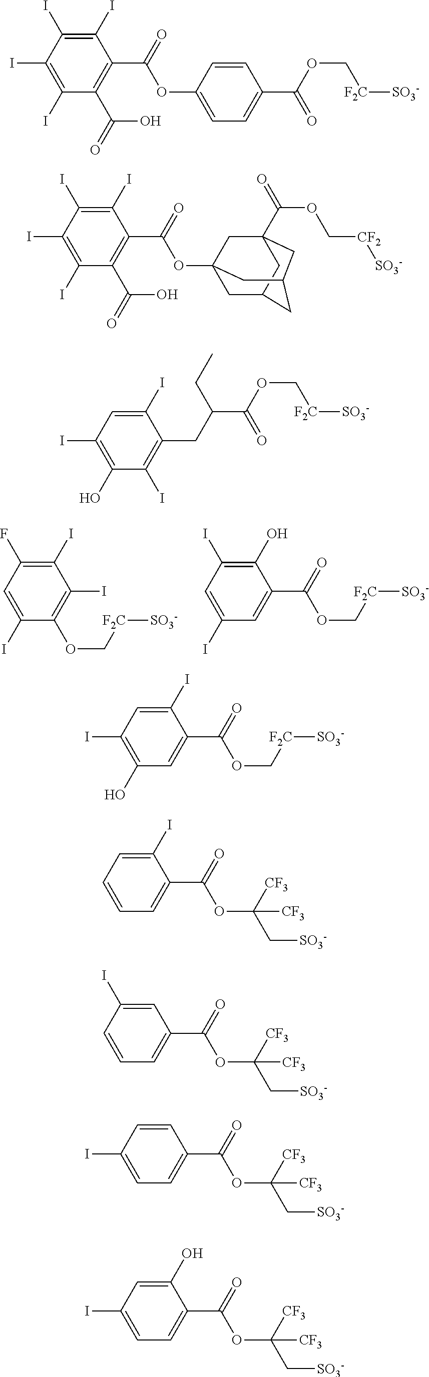

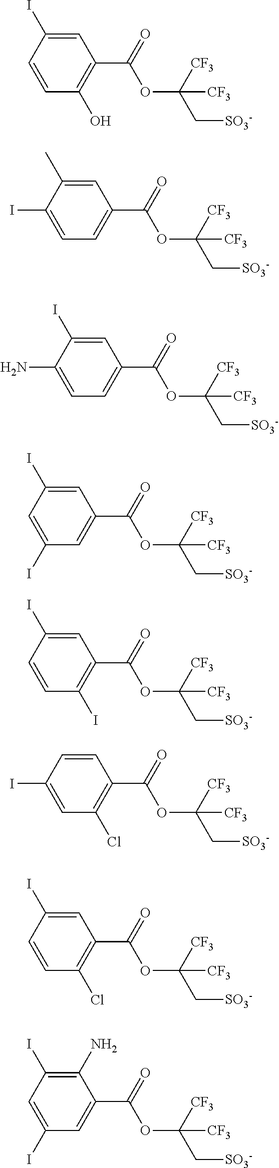

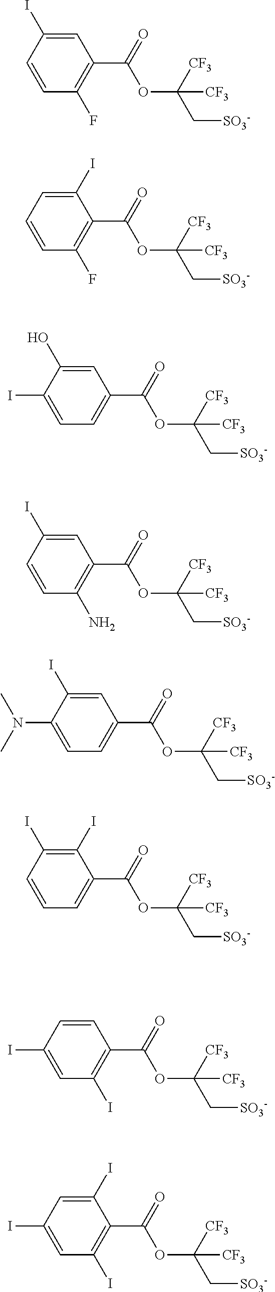

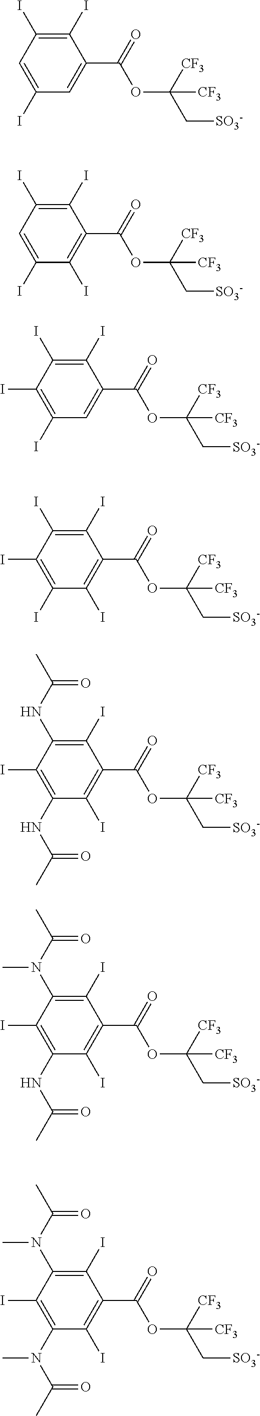

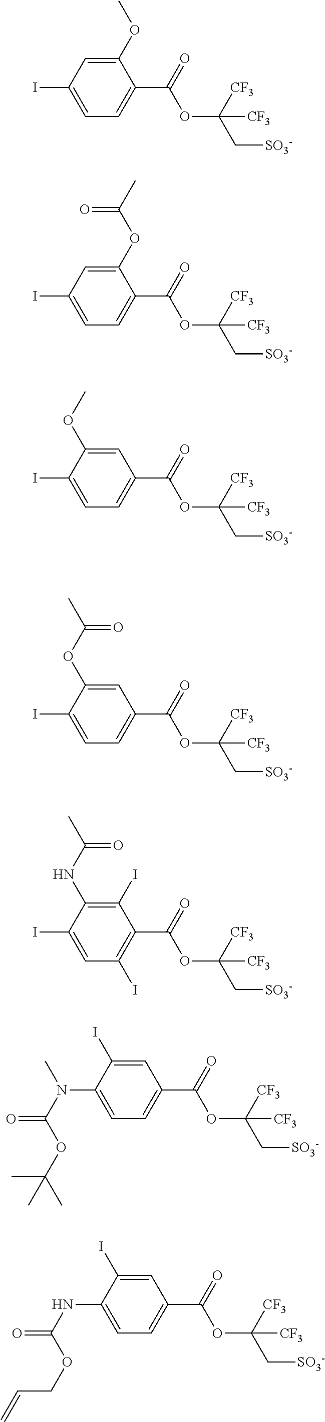

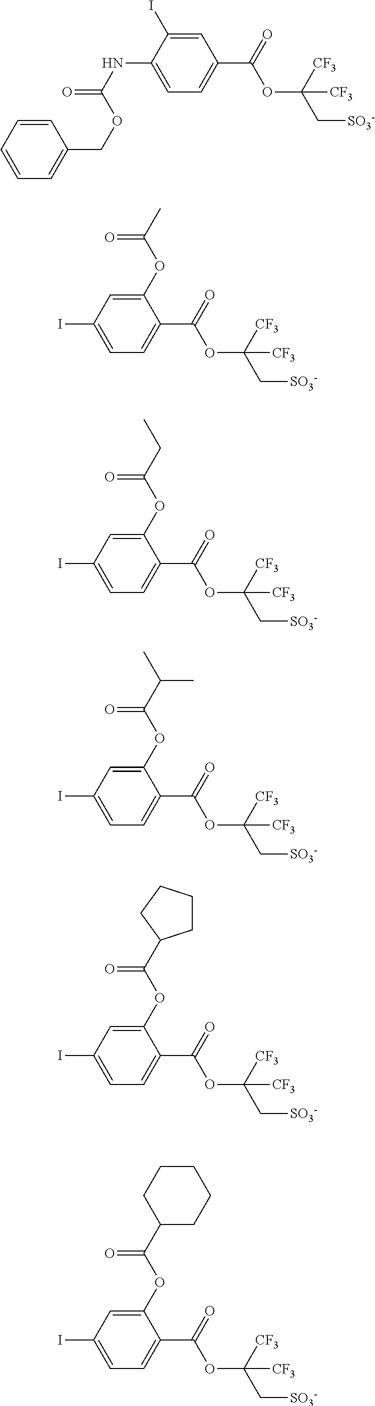

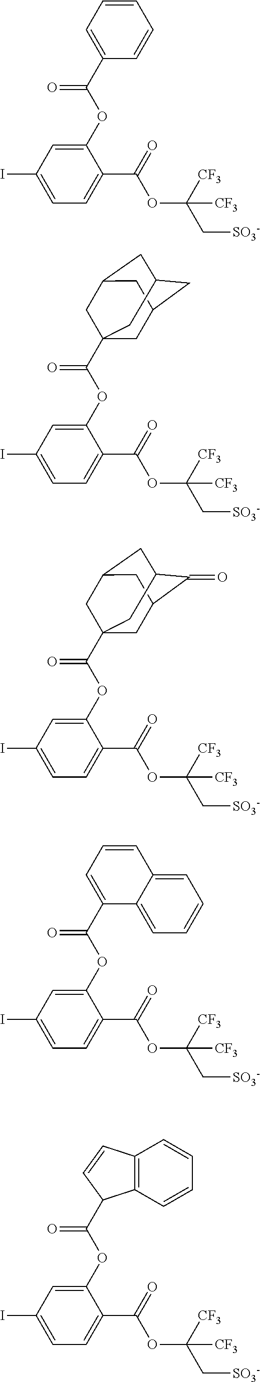

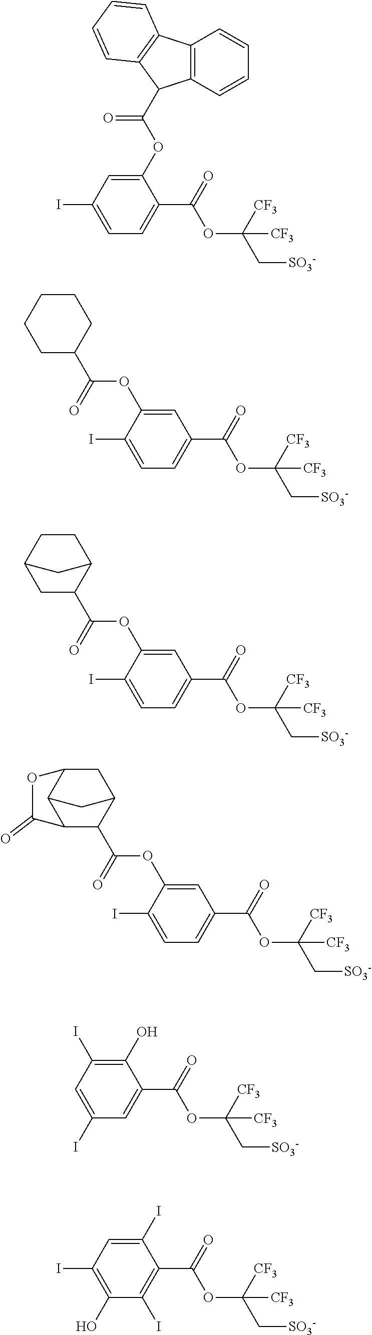

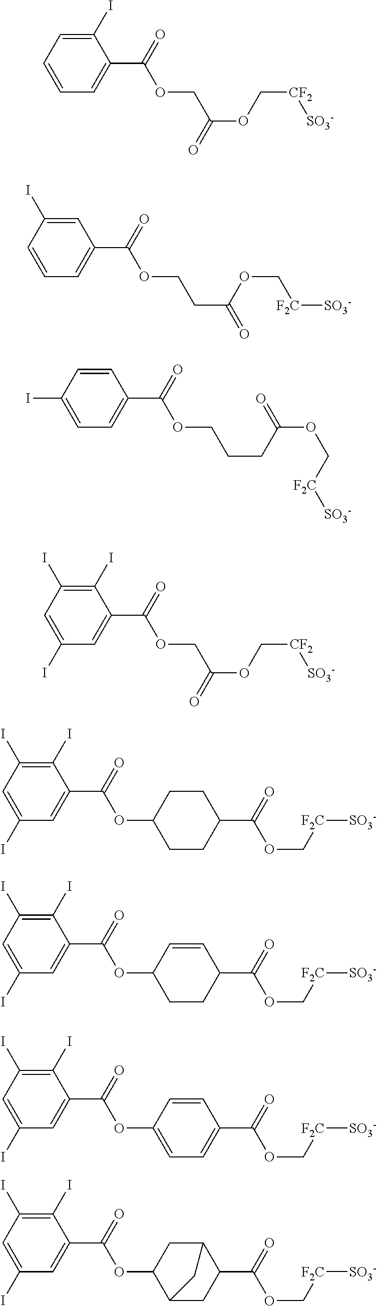

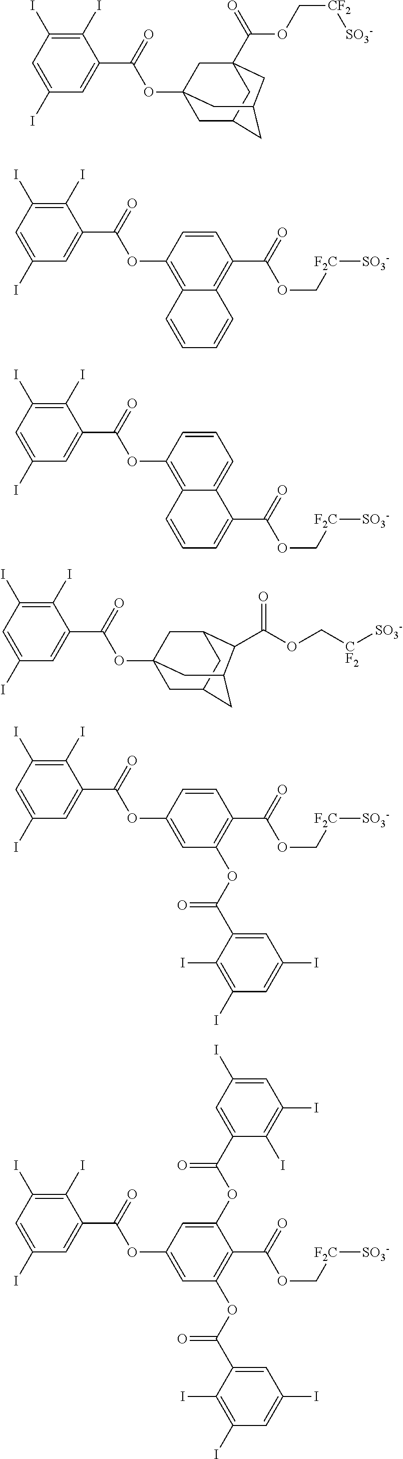

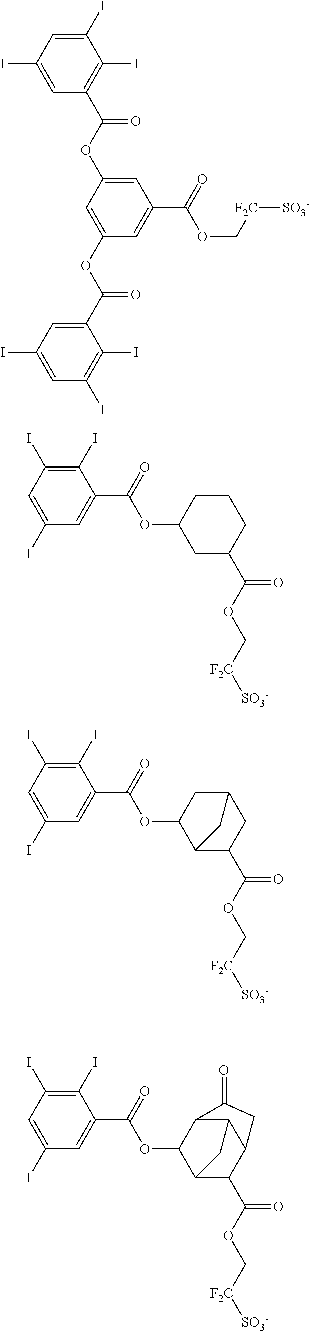

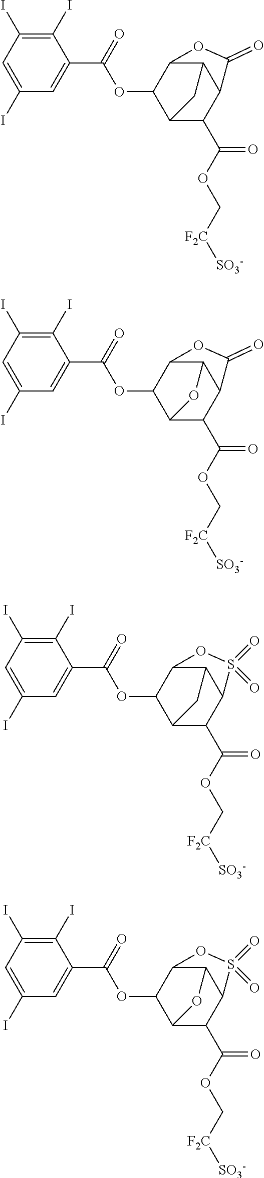

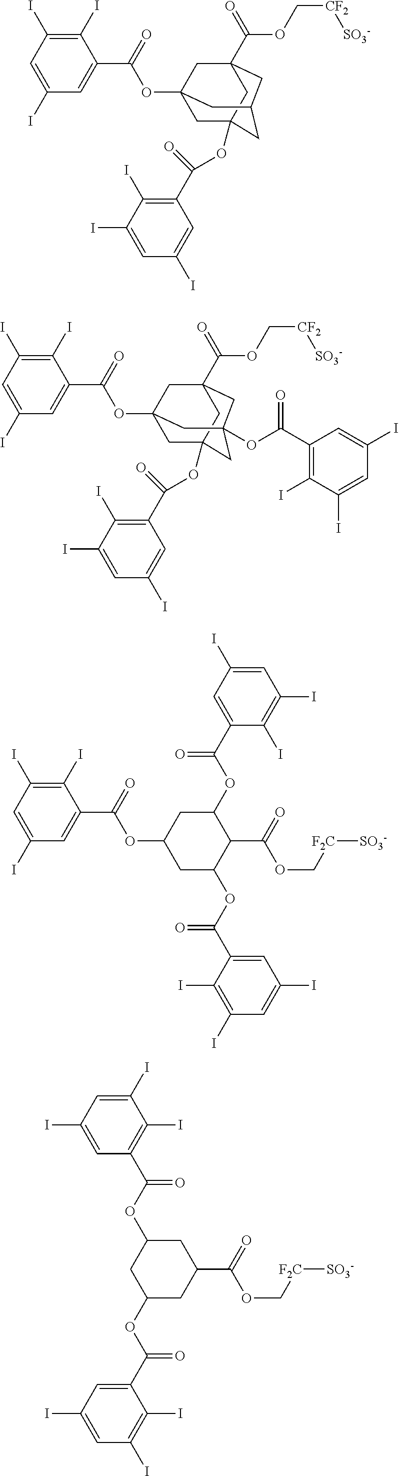

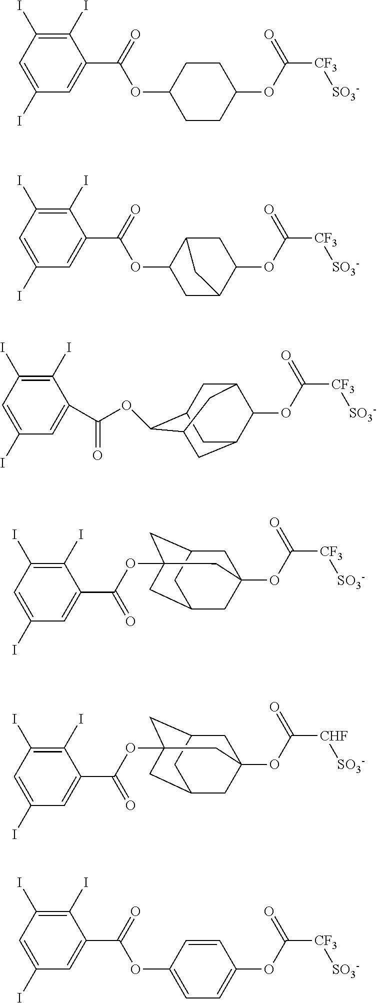

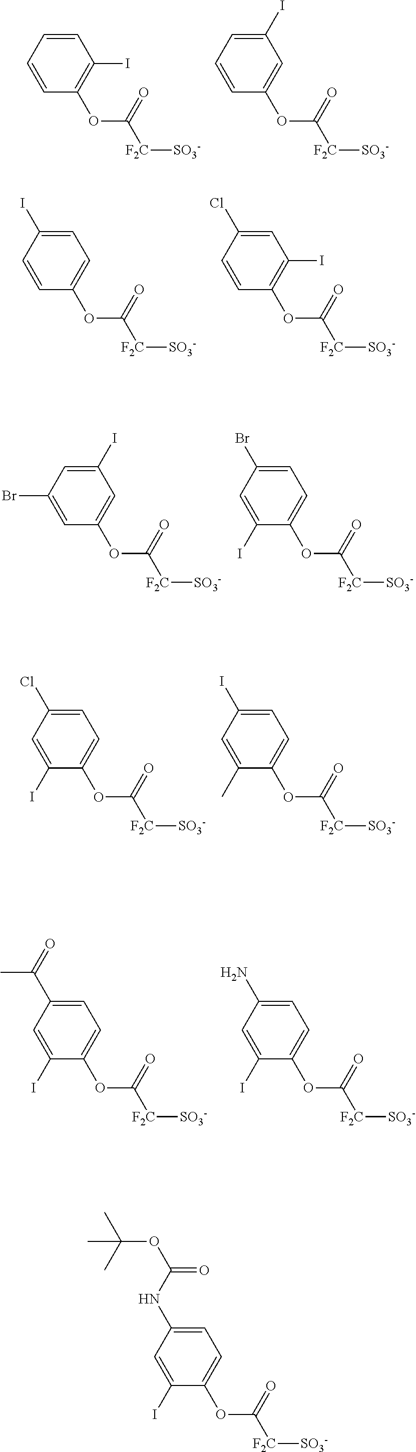

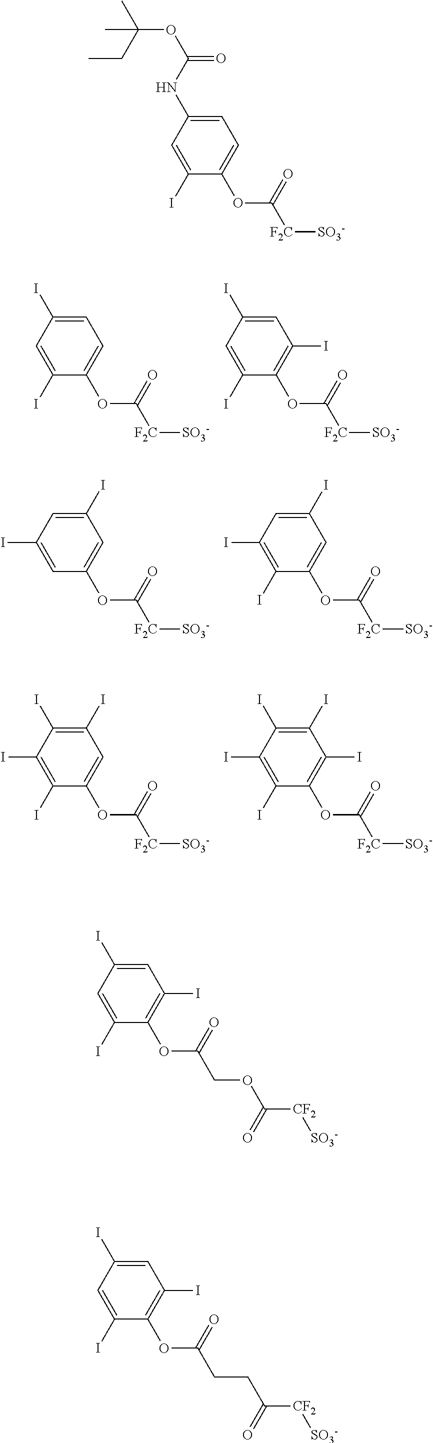

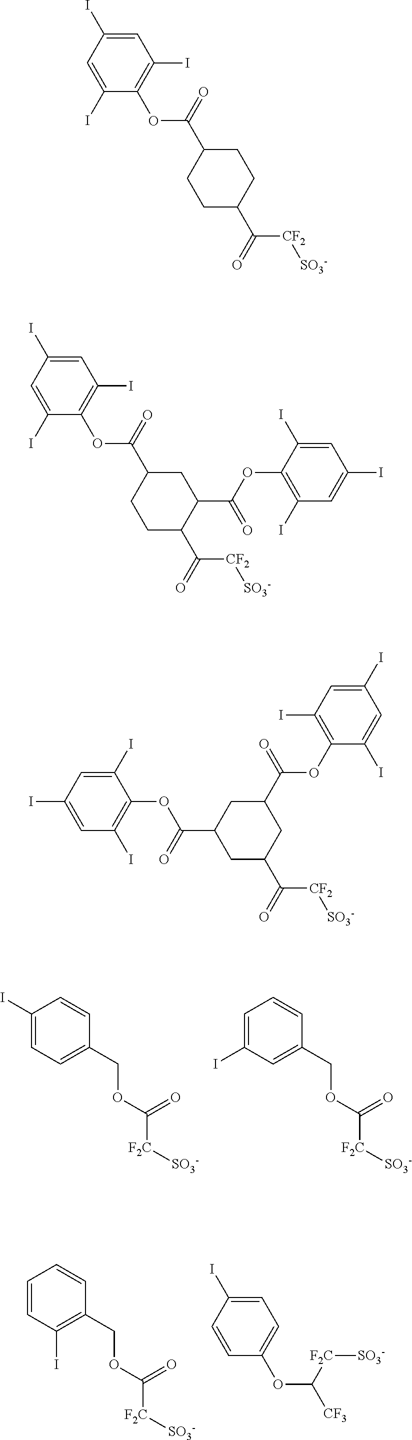

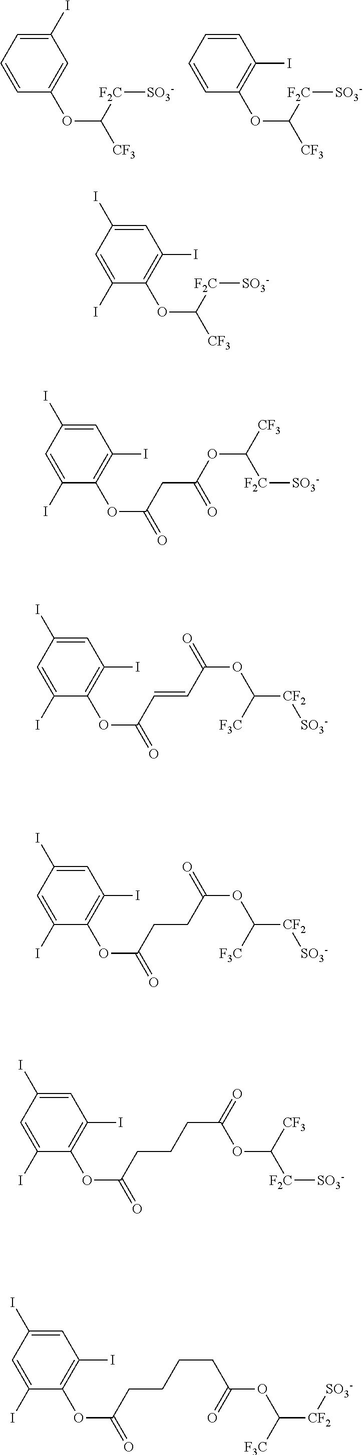

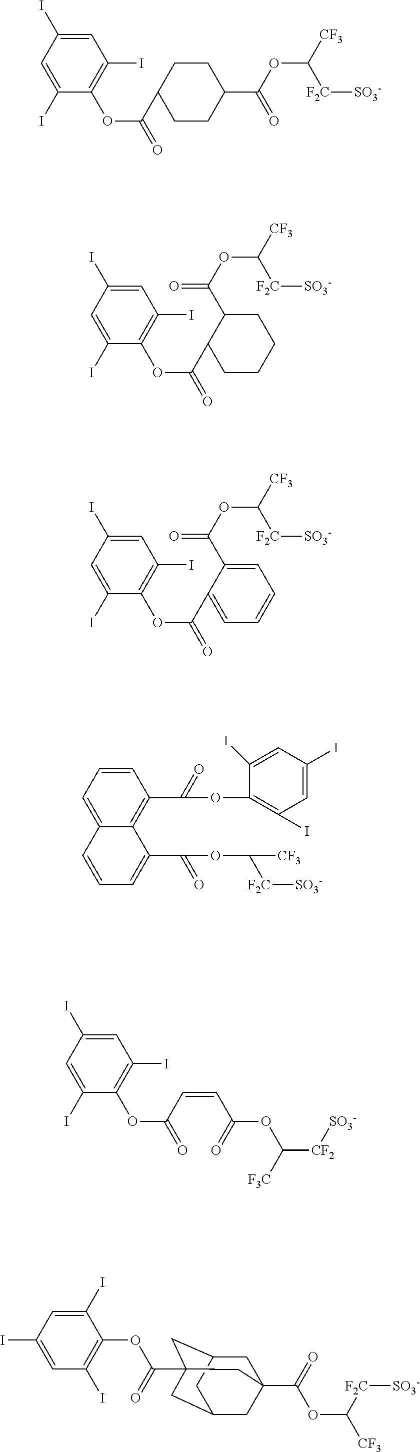

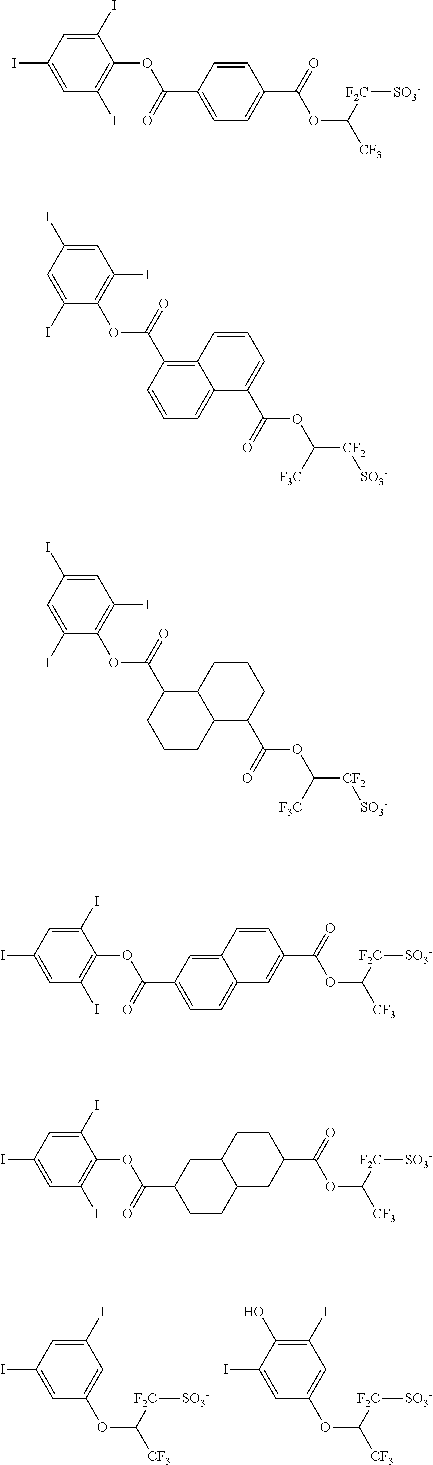

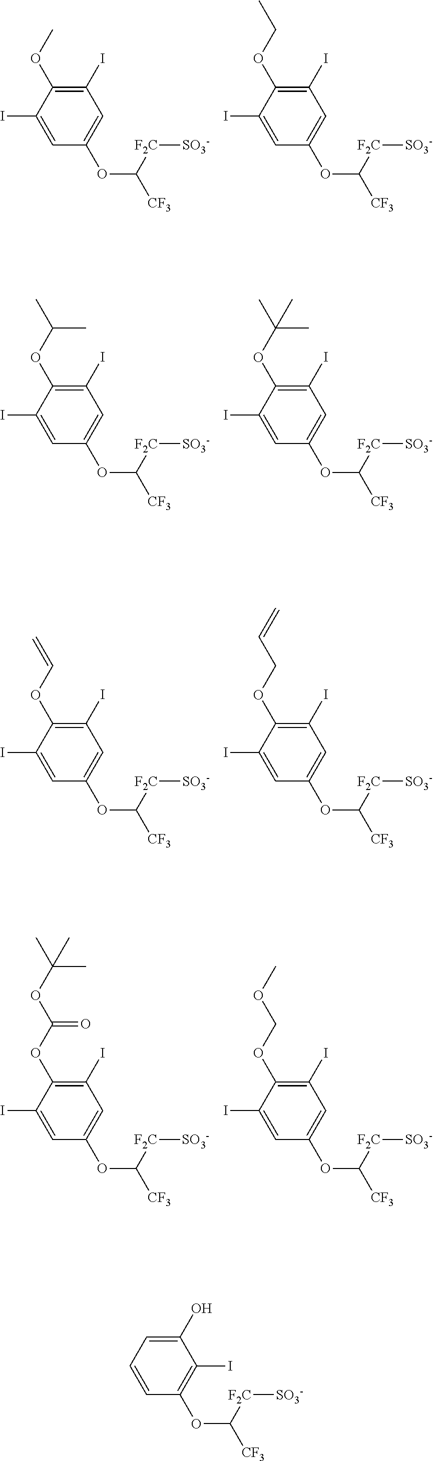

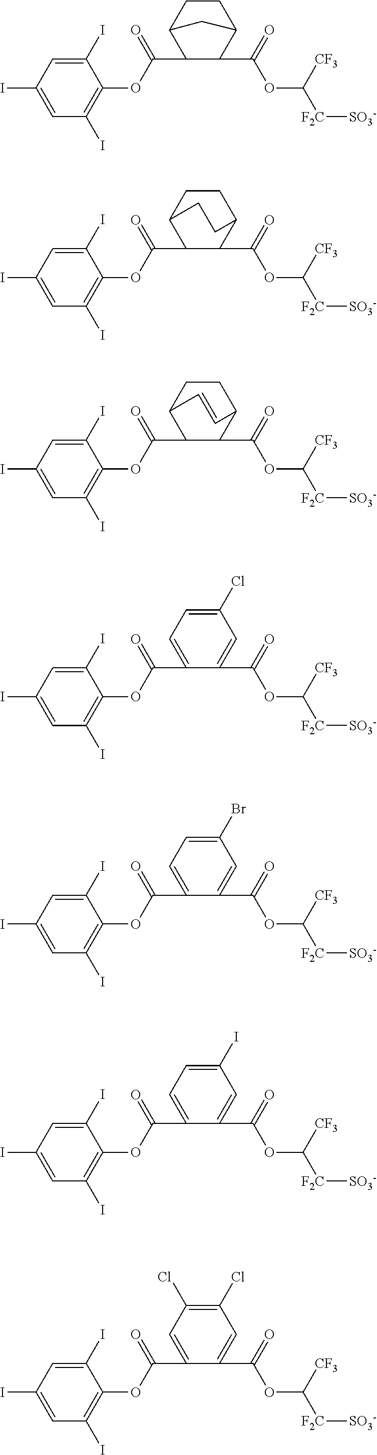

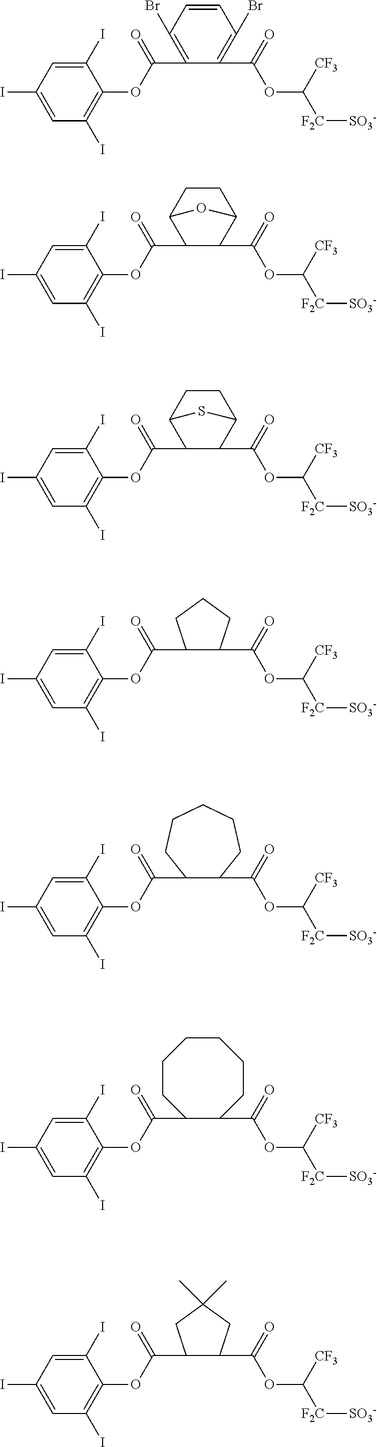

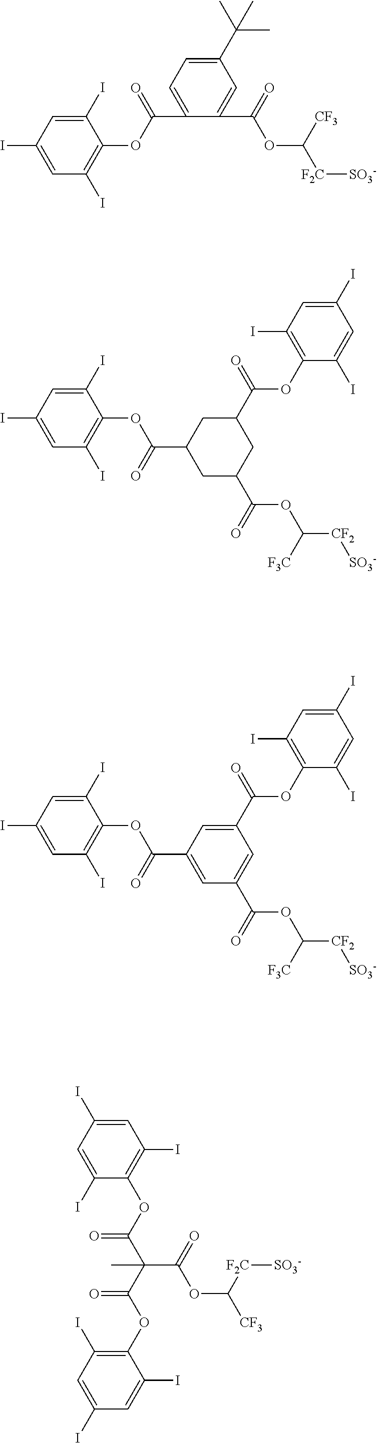

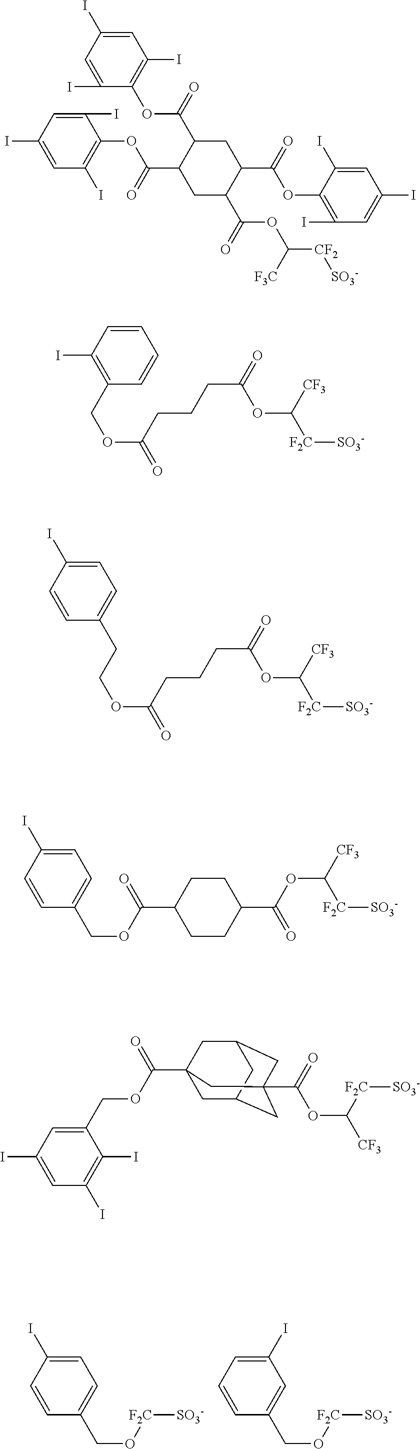

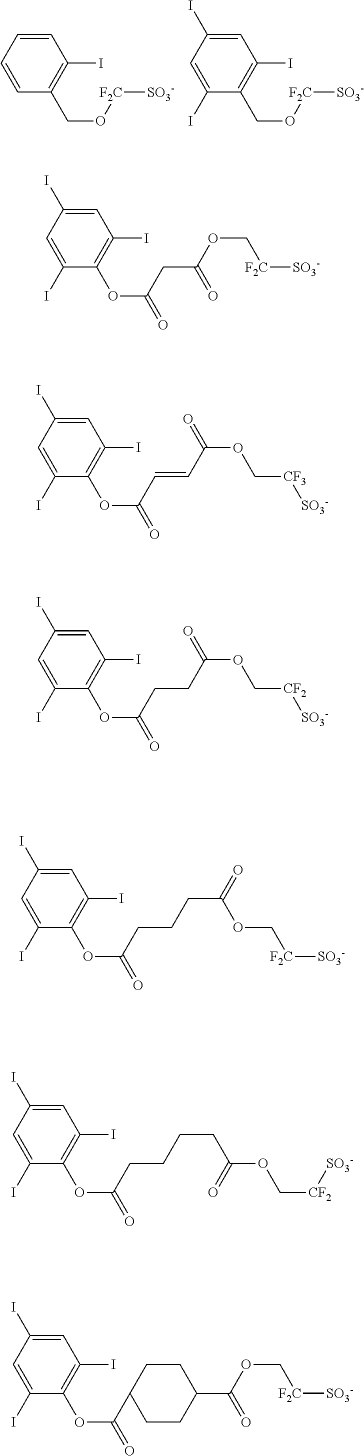

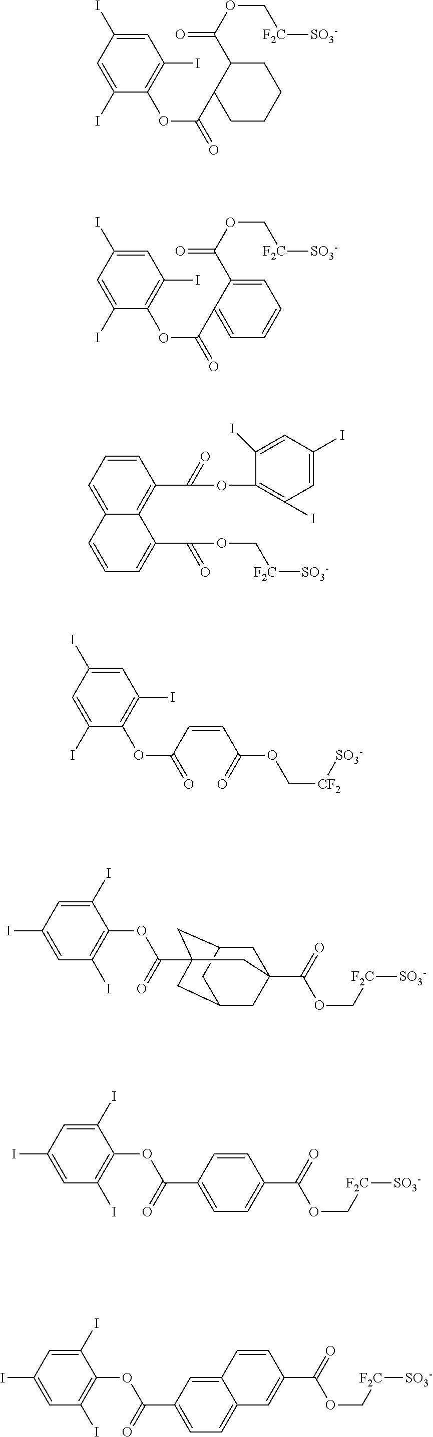

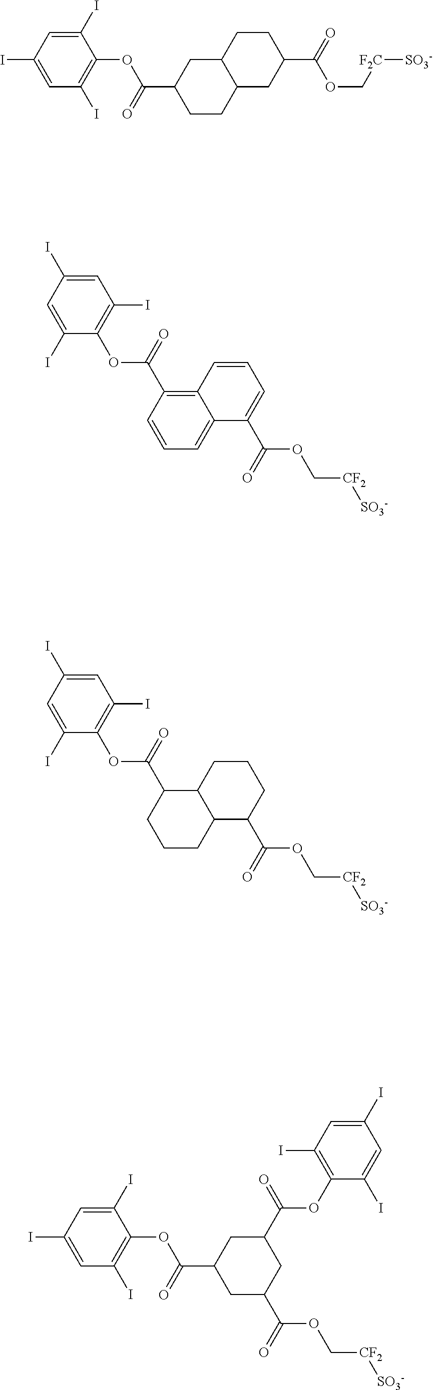

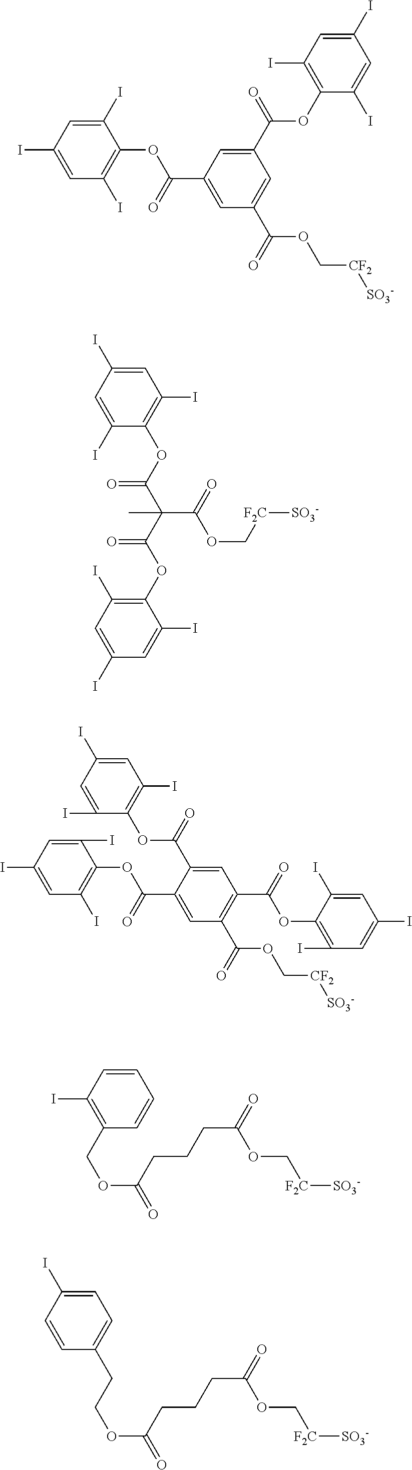

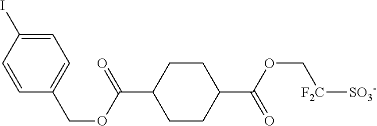

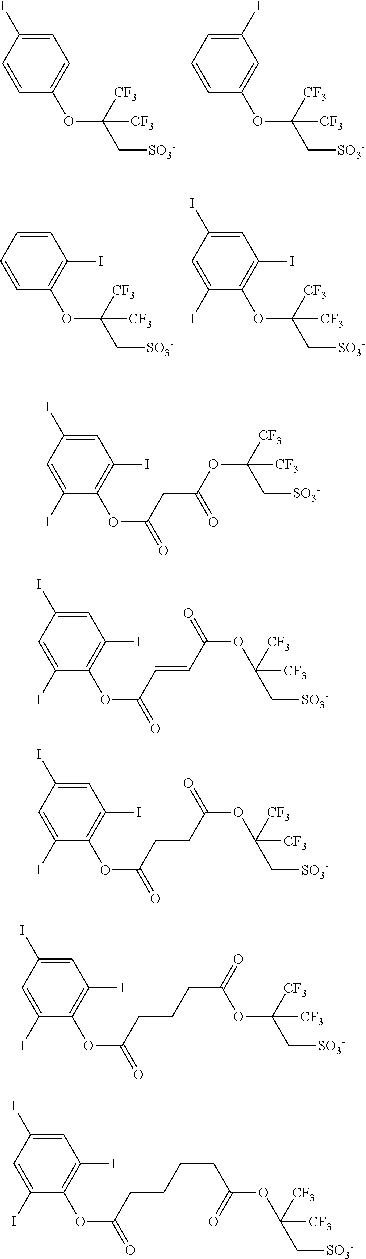

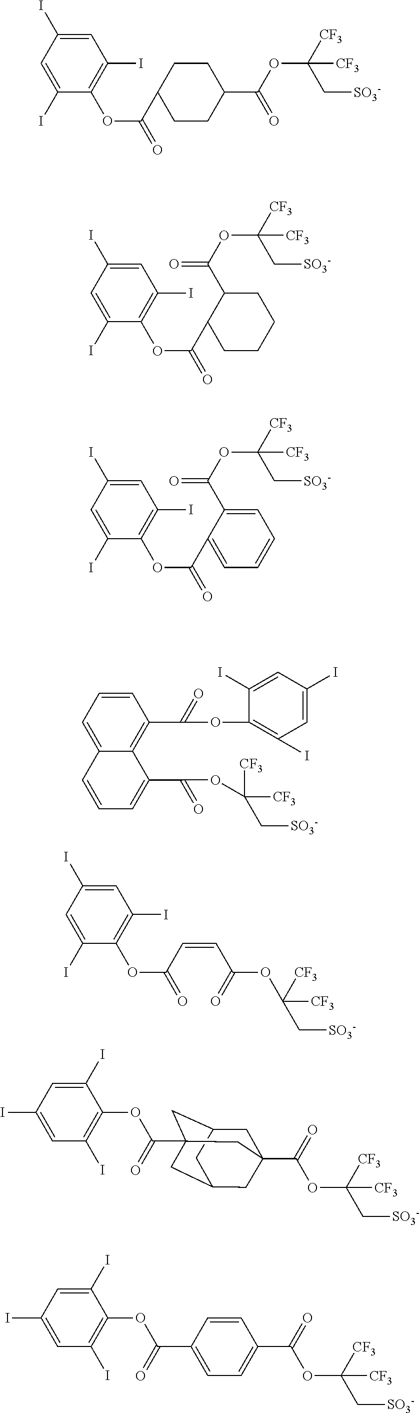

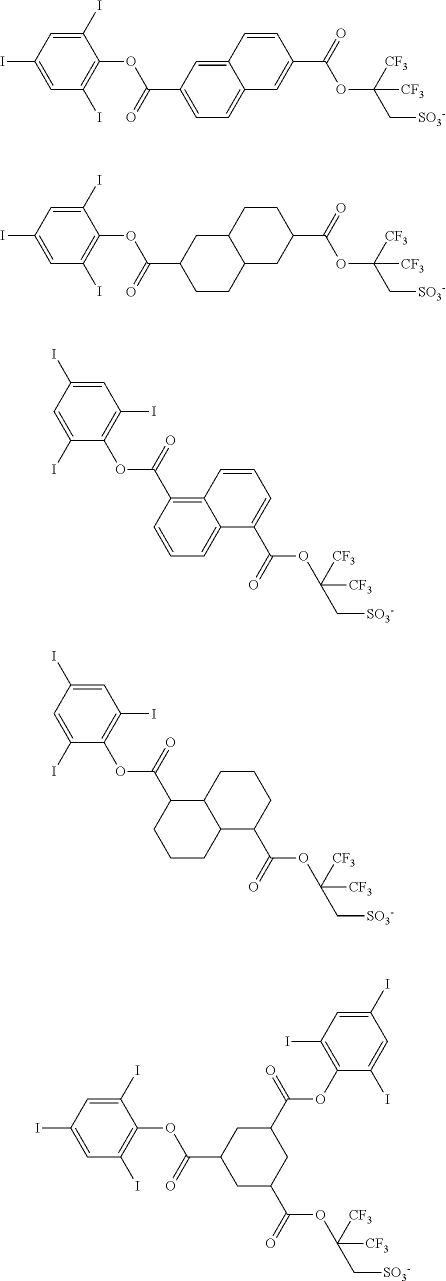

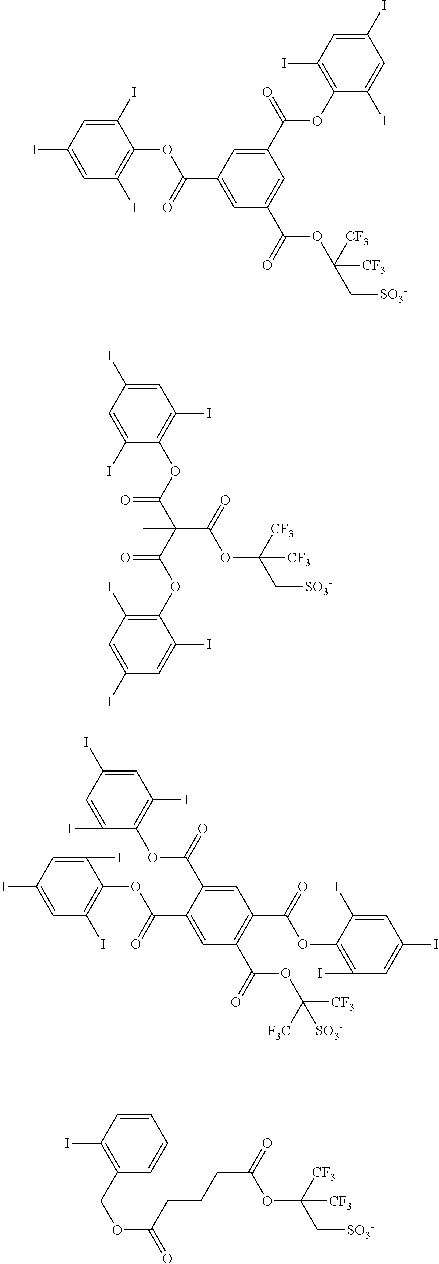

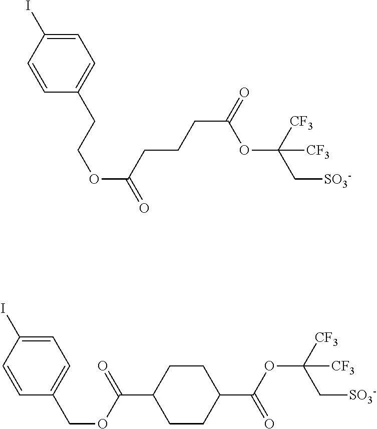

When the inventive chemically amplified resist composition comprises acid generator (D), the amount of the acid generator (D) used is preferably 0.1 to 40 parts by weight, more preferably 0.5 to 20 parts by weight per 80 parts by weight of the base polymer (A). An amount of the acid generator (D) in the range ensures good resolution and eliminates the risk of leaving foreign matter after development or during separation of resist film. The acid generator (D) may be used alone or in admixture.

### (E) Surfactant

The inventive chemically amplified resist composition may further comprise (E) a surfactant. It is preferably (E) a surfactant which is insoluble or substantially insoluble in water but soluble in alkaline developer, or a surfactant which is insoluble or substantially insoluble in water and alkaline developer. For the surfactant, reference should be made to those compounds described in JP-A 2010-215608 and JP-A 2011-16746.

While many examples of the surfactant which is insoluble or substantially insoluble in water and alkaline developer are described in the patent documents cited herein, preferred examples are fluorochemical surfactants FC-4430 (3M), Olfine® E1004 (Nissin Chemical Co., Ltd.), Surflon® S-381, KH-20 and KH-30 (AGC Seimi Chemical Co., Ltd.). Partially fluorinated oxetane ring-opened polymers having the formula (surf-1) are also useful.

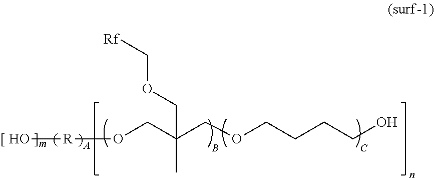

It is provided herein that R, Rf, A, B, C, m, and n are applied to only formula (surf-1), independent of the above descriptions. R is a di- to tetra-valent C₂-C₅ aliphatic group. Exemplary divalent aliphatic groups include ethylene, 1,4-butylene, 1,2-propylene, 2,2-dimethyl-1,3-propylene and 1,5-pentylene. Exemplary tri- and tetra-valent groups are shown below.

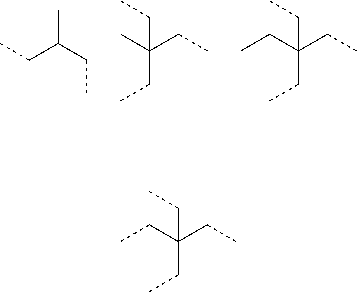

Herein the broken line denotes a valence bond. These formulae are partial structures derived from glycerol, trimethylol ethane, trimethylol propane, and pentaerythritol, respectively.

Of these, 1,4-butylene and 2,2-dimethyl-1,3-propylene are preferred.

Rf is trifluoromethyl group or pentafluoroethyl group, preferably trifluoromethyl group. The subscript m is an integer of 0 to 3, n is an integer of 1 to 4, and the sum of m and n, which represents the valence of R, is an integer of 2 to 4. A is 1. B is an integer of 2 to 25, preferably an integer of 4 to 20. C is an integer of 0 to 10, preferably 0 or 1. Note that formula (surf-1) does not prescribe the arrangement of respective constituent units while they may be arranged either blockwise or randomly. For the preparation of surfactants in the form of partially fluorinated oxetane ring-opened polymers, reference should be made to U.S. Pat. No. 5,650,483, for example.

The surfactant which is insoluble or substantially insoluble in water and soluble in alkaline developer is useful when ArF immersion lithography is applied to the resist composition in the absence of a resist protective film. In this embodiment, the surfactant has a propensity to segregate on the surface of a resist film for achieving a function of minimizing water penetration or leaching. The surfactant is also effective for preventing water-soluble components from being leached out of the resist film for minimizing any damage to the exposure tool. The surfactant becomes solubilized during alkaline development following exposure and PEB, and thus forms few or no foreign matter which becomes defects. The preferred surfactant is a polymeric surfactant which is insoluble or substantially insoluble in water, but soluble in alkaline developer, also referred to as “hydrophobic resin” in this sense, and especially which is water repellent and enhances water sliding.

Examples of the polymeric surfactant include those containing repeat units of at least one type selected from the formulae (6A) to (6E).

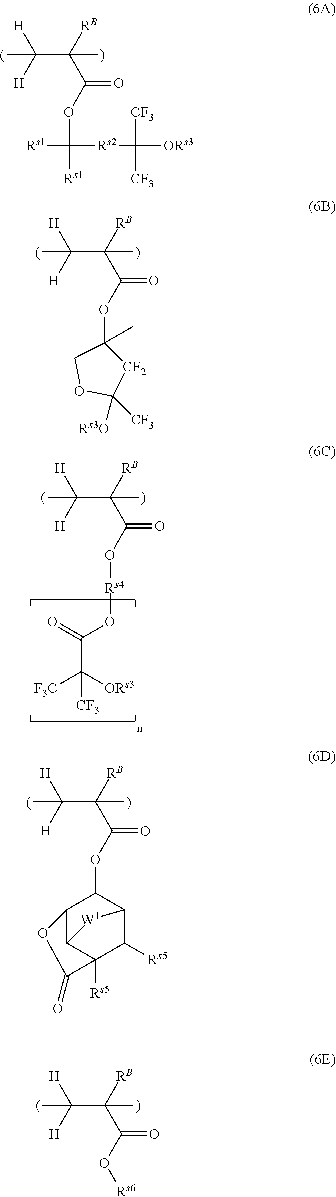

In formulae (6A) to (6E), R^{B} is hydrogen, fluorine, a methyl group, or a trifluoromethyl group. W¹ is —CH₂—, —CH₂CH₂— or —O—, or two separate —H. R^{s1} is each independently hydrogen or a C₁-C₁₀ hydrocarbyl group. R^{s2} is a single bond or C₁-C₅ straight or branched hydrocarbylene group. R^{s3} is each independently hydrogen, a C₁-C₁₅ hydrocarbyl or fluorinated hydrocarbyl group, or an acid labile group. When R^{s3} is a hydrocarbyl or fluorinated hydrocarbyl group, an ether bond or carbonyl moiety may intervene in a carbon-carbon bond. R^{s4} is a C₁-C₂₀ (u+1)-valent hydrocarbon or fluorinated hydrocarbon group. The subscript u is 1, 2 or 3. R^{s5} is each independently hydrogen or a group: —C(═O)—O—R^{sa}. R^{sa} is a C₁-C₂₀ fluorinated hydrocarbyl group. R^{s6} is a C₁-C₁₅ hydrocarbyl or fluorinated hydrocarbyl group in which an ether bond or carbonyl moiety may intervene in a carbon-carbon bond.

The C₁-C₁₀ hydrocarbyl group R^{s1} is preferably saturated while it may be straight, branched or cyclic. Examples thereof include C₁-C₁₀ alkyl groups such as methyl, ethyl, n-propyl, isopropyl, n-butyl, isobutyl, sec-butyl, tert-butyl, n-pentyl, n-hexyl, n-heptyl, n-octyl, n-nonyl and n-decyl, and C₃-C₁₀ cyclic saturated hydrocarbyl groups such as cyclopropyl, cyclobutyl, cyclopentyl, cyclohexyl, adamantyl and norbornyl. Inter alia, C₁-C₆ hydrocarbyl groups are preferred.

The hydrocarbylene group R^{s2} is preferably saturated while it may be straight, branched or cyclic. Examples thereof include methylene, ethylene, propylene, butylene and pentylene groups.

The hydrocarbyl group R^{s3} or R^{s6} may be saturated or unsaturated and straight, branched or cyclic. Examples thereof include saturated hydrocarbyl groups and aliphatic unsaturated hydrocarbyl groups such as alkenyl and alkynyl groups, with the saturated hydrocarbyl groups being preferred. Examples of the saturated hydrocarbyl groups include those exemplified for the hydrocarbyl group R^{s1} as well as undecyl, dodecyl, tridecyl, tetradecyl, and pentadecyl groups. Examples of the fluorinated hydrocarbyl group R^{s3} or R^{s6} include the foregoing hydrocarbyl groups in which some or all carbon-bonded hydrogen atoms are substituted by fluorine atoms. In these groups, an ether bond or carbonyl moiety may intervene in a carbon-carbon bond as mentioned above.

Examples of the acid labile group R^{s3} include the groups of formulae (AL-3) to (AL-5), trialkylsilyl groups in which each alkyl group is a C₁-C₆ alkyl group, and C₄-C₂₀ oxoalkyl groups.

The (u+1)-valent hydrocarbon or fluorinated hydrocarbon group R^{s4} may be straight, branched or cyclic, and examples thereof include the foregoing hydrocarbyl or fluorinated hydrocarbyl groups from which “u” number of hydrogen atoms are eliminated.

The fluorinated hydrocarbyl group R^{sa} is preferably saturated while it may be straight, branched or cyclic. Examples thereof include the foregoing hydrocarbyl groups in which some or all hydrogen atoms are substituted by fluorine atoms. Illustrative examples include trifluoromethyl, 2,2,2-trifluoroethyl, 3,3,3-trifluoro-1-propyl, 3,3,3-trifluoro-2-propyl, 2,2,3,3-tetrafluoropropyl, 1,1,1,3,3,3-hexafluoroisopropyl, 2,2,3,3,4,4,4-heptafluorobutyl, 2,2,3,3,4,4,5,5-octafluoropentyl, 2,2,3,3,4,4,5,5,6,6,7,7-dodecafluoroheptyl, 2-(perfluorobutyl)ethyl, 2-(perfluorohexyl)ethyl, 2-(perfluorooctyl)ethyl, and 2-(perfluorodecyl)ethyl.

Examples of the repeat units of formulae (6A) to (6E) are shown below, but not limited thereto. Herein R^{B} is as defined above.

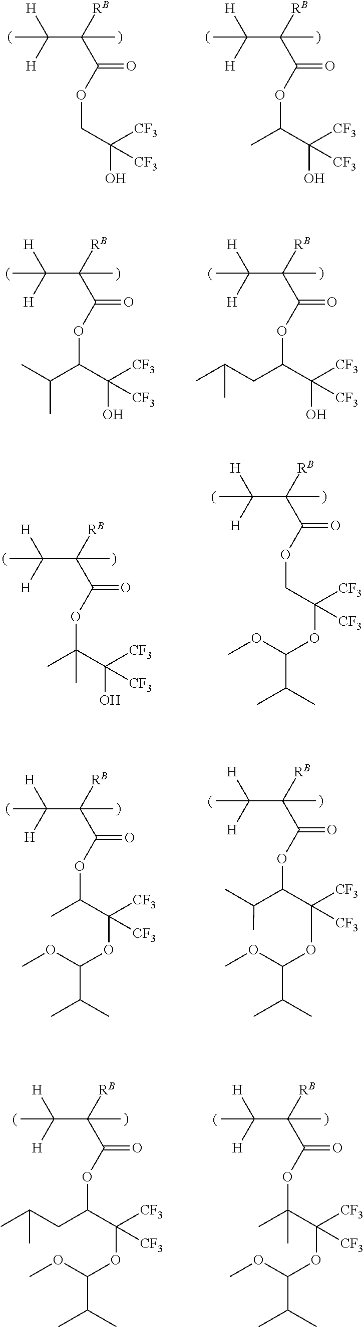

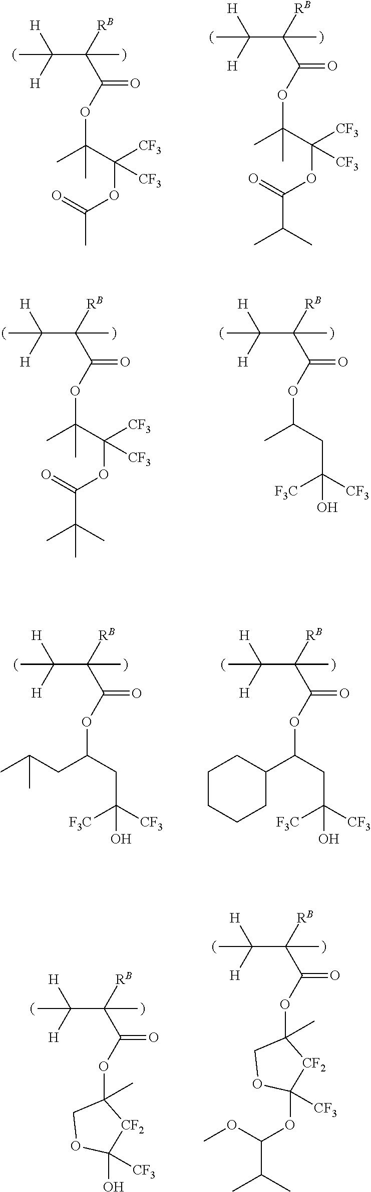

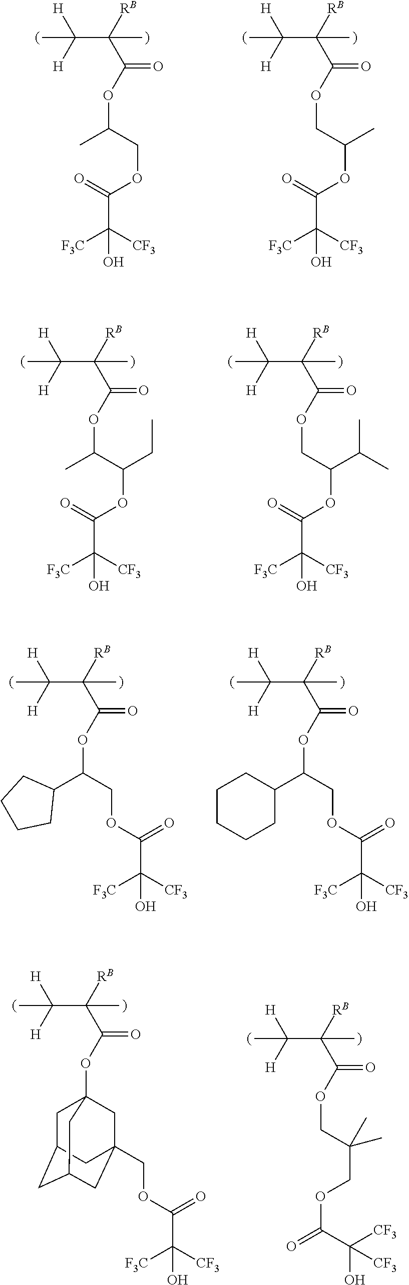

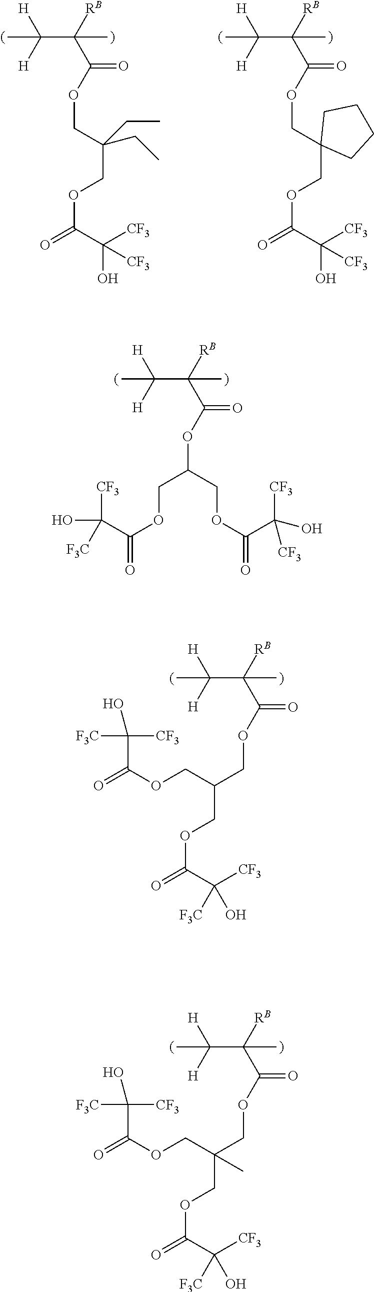

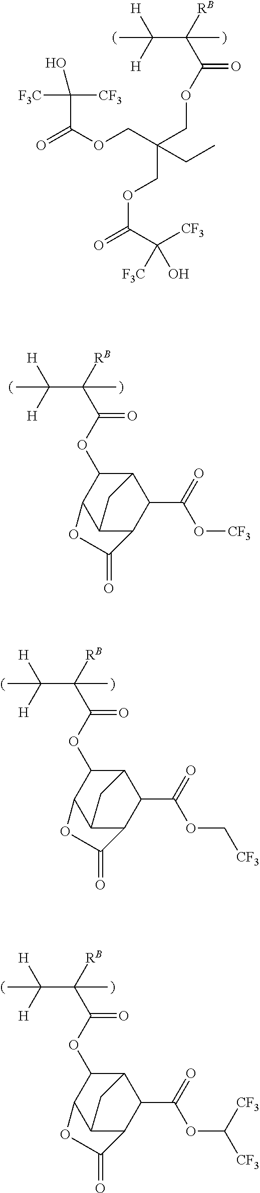

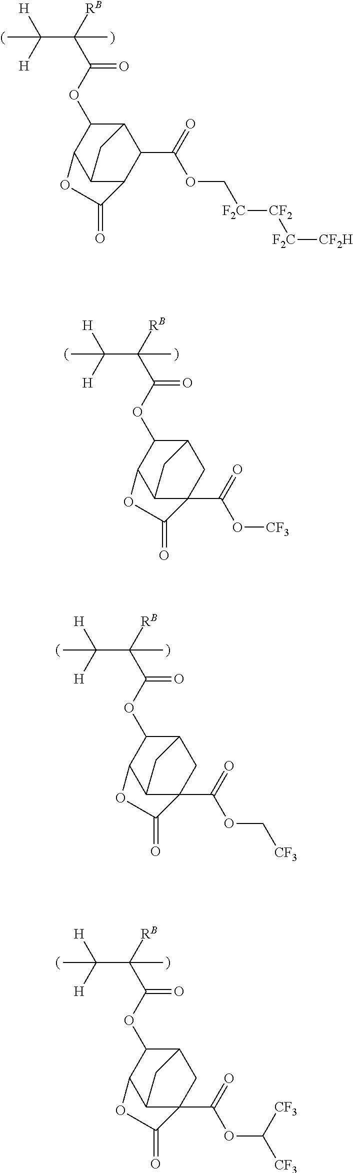

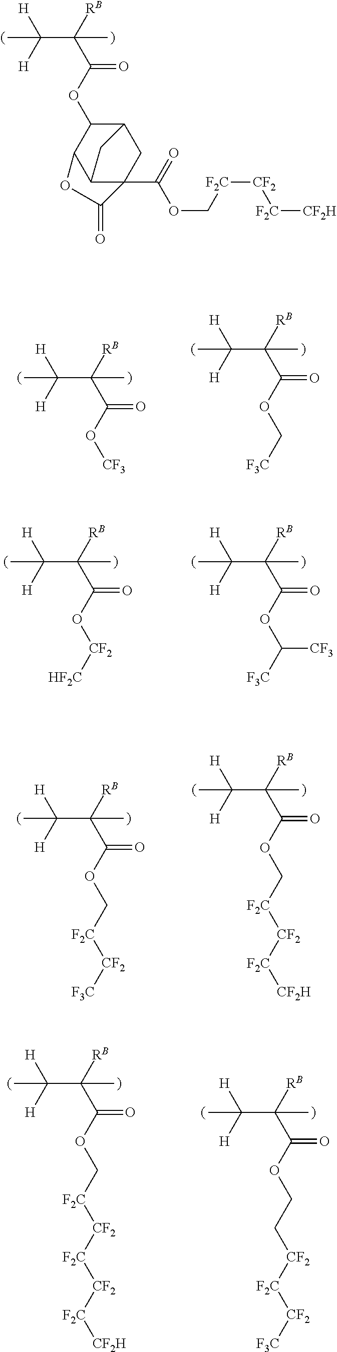

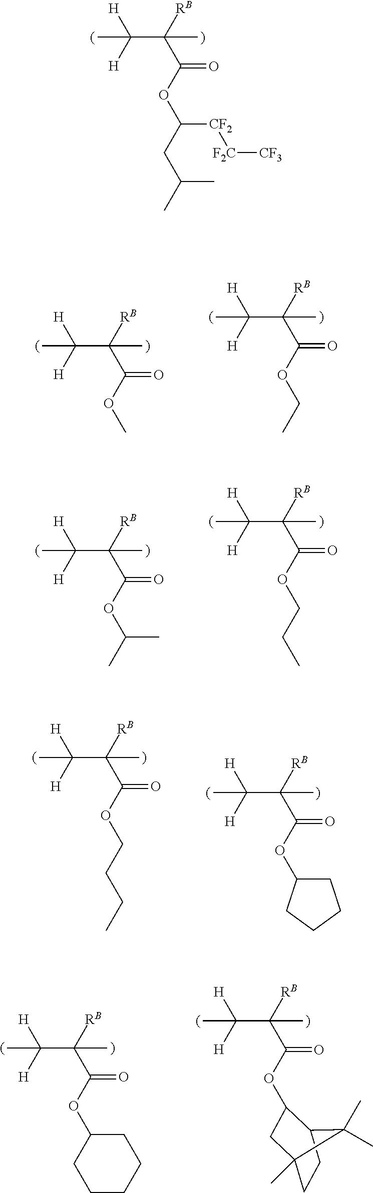

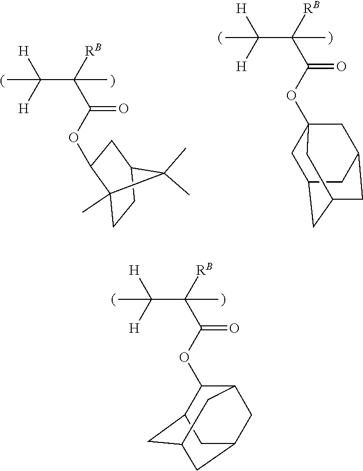

The polymeric surfactant may further contain repeat units exclusive of the repeat units having formulae (6A) to (6E). Typical other repeat units are, for example, those derived from methacrylic acid and α-trifluoromethylacrylic acid derivatives. In the polymeric surfactant, the content of the repeat units having formulae (6A) to (6E) is preferably at least 20 mol %, more preferably at least 60 mol %, most preferably 100 mol % of the overall repeat units.

Mw of the polymeric surfactant is preferably 1,000 to 500,000, more preferably 3,000 to 100,000. Mw/Mn is preferably 1.0 to 2.0, more preferably 1.0 to 1.6.

The polymeric surfactant may be synthesized, for example, by dissolving an unsaturated bond-containing monomer or monomers, from which repeat units having formulae (6A) to (6E) and optional other repeat units are derived, in an organic solvent, adding a radical initiator, and heating for polymerization. Examples of the suitable organic solvent used herein include toluene, benzene, THF, diethyl ether, and dioxane. Examples of the polymerization initiator used herein include AIBN, 2,2′-azobis(2,4-dimethylvaleronitrile), dimethyl 2,2-azobis(2-methylpropionate), benzoyl peroxide, and lauroyl peroxide. The reaction temperature is preferably 50 to 100° C. The reaction time is preferably 4 to 24 hours. The acid labile group that has been incorporated in the monomer may be kept as such, or the polymerization may be followed by protection or partial protection.

During the synthesis of the polymeric surfactant, any of well-known chain transfer agents such as dodecylmercaptan and 2-mercaptoethanol may be used for the purpose of adjusting molecular weight. An appropriate amount of the chain transfer agent is 0.01 to 10 mol % based on the total moles of monomers to be polymerized.

When the chemically amplified resist composition contains the surfactant (E), the amount of the surfactant (E) used is 0.1 to 50 parts by weight, more preferably 0.5 to 10 parts by weight per 80 parts by weight of the base polymer (A). As long as the amount of the surfactant (E) is at least 0.1 parts by weight, the receding contact angle of resist film surface with water is fully improved. As long as the amount of the surfactant (E) is up to 50 parts by weight, the dissolution rate of resist film surface in developer is so low that the resulting small-size pattern may maintain a sufficient height. The surfactant (E) may be used alone or in admixture.

### (F) Dissolution Inhibitor

The inventive chemically amplified resist composition may further comprise (F) a dissolution inhibitor. In the case of positive resist compositions, the inclusion of a dissolution inhibitor may lead to an increased difference in dissolution rate between exposed and unexposed areas and a further improvement in resolution.

The dissolution inhibitor which can be used herein is a compound having at least two phenolic hydroxy groups on the molecule, in which an average of from 0 to 100 mol % of all the hydrogen atoms on the phenolic hydroxy groups are replaced by acid labile groups or a compound having at least one carboxy group on the molecule, in which an average of 50 to 100 mol % of all the hydrogen atoms on the carboxy groups are replaced by acid labile groups, both the compounds having a molecular weight of 100 to 1,000, and preferably 150 to 800. Typical are bisphenol A, trisphenol, phenolphthalein, cresol novolac, naphthalenecarboxylic acid, adamantanecarboxylic acid, and cholic acid derivatives in which the hydrogen atom on the hydroxy or carboxy group is replaced by an acid labile group, as described in U.S. Pat. No. 7,771,914 (JP-A 2008-122932, paragraphs [0155]-[0178]).

In the inventive chemically amplified resist composition, the dissolution inhibitor (F) is preferably added in an amount of 0 to 50 parts, more preferably 5 to 40 parts by weight per 80 parts by weight of the base polymer (A). The dissolution inhibitor (F) may be used alone or in admixture.

### (G) Other Components

The inventive chemically amplified resist composition may further comprise (G) another component, for example, a compound which is decomposed with an acid to generate another acid (i.e., acid amplifier compound), organic acid derivative, fluorinated alcohol, and water repellency improver. The acid amplifier compound is described in JP-A 2009-269953 and JP-A 2010-215608. The acid amplifier compound is preferably used in an amount of 0 to 5 parts, more preferably 0 to 3 parts by weight per 80 parts by weight of the base polymer (A). An extra amount of the acid amplifier compound can make the acid diffusion control difficult and cause degradations to resolution and pattern profile. With respect to the organic acid derivative and fluorinated alcohol, reference should be made to JP-A 2009-269953 and JP-A 2010-215608.

The water repellency improver may be used in the topcoatless immersion lithography. Suitable water repellency improvers include polymers having a fluoroalkyl group and polymers having a specific structure with a 1,1,1,3,3,3-hexafluoro-2-propanol residue and are described in JP-A 2007-297590 and JP-A 2008-111103, for example. The water repellency improver should be soluble in alkaline developers and organic solvent developers. The water repellency improver of specific structure with a 1,1,1,3,3,3-hexafluoro-2-propanol residue is well soluble in the developer. A polymer having an amino group or amine salt copolymerized as repeat units may serve as the water repellency improver and is effective for preventing evaporation of acid during PEB, thus preventing any hole pattern opening failure after development. When the inventive chemically amplified resist composition comprises the water repellency improved, the amount of the water repellency improver used is preferably 0 to 20 parts by weight, more preferably 0.5 to 10 parts by weight per 80 parts by weight of the base polymer (A).

### Pattern Forming Process

The inventive chemically amplified resist composition is used in the fabrication of various integrated circuits. Pattern formation using the chemically amplified resist composition may be performed by well-known lithography processes. The process generally involves the steps of applying the chemically amplified resist composition onto a substrate to form a resist film thereon, exposing the resist film to high-energy radiation, and developing the exposed resist film in a developer.

The inventive chemically amplified resist composition is first applied onto a substrate on which an integrated circuit is to be formed (e.g., Si, SiO₂, SiN, SiON, TiN, WSi, BPSG, SOG, or organic antireflective coating) or a substrate on which a mask circuit is to be formed (e.g., Cr, CrO, CrON, MoSi₂, or SiO₂) by a suitable coating technique such as spin coating, roll coating, flow coating, dipping, spraying or doctor coating. The resulting resist film is generally 0.01 to 2.0 μm thick. The coating is prebaked on a hotplate preferably at a temperature of 60 to 150° C. for 10 seconds to 30 minutes, and more preferably at 80 to 120° C. for 30 seconds to 20 minutes to form a resist film.

Then the resist film is exposed to high-energy radiation. Examples of the high-energy radiation include UV, deep-UV, EB, EUV of wavelength 3 to 15 nm, x-ray, soft x-ray, excimer laser light, γ-ray or synchrotron radiation. On use of UV, deep UV, EUV, x-ray, soft x-ray, excimer laser, γ-ray or synchrotron radiation, the resist film is exposed directly or through a mask having a desired pattern, preferably in a dose of about 1 to 200 mJ/cm², more preferably about 10 to 100 mJ/cm². On use of EB, a pattern may be written directly or through a mask having a desired pattern, preferably in a dose of about 0.1 to 100 μC/cm², more preferably about 0.5 to 50 μC/cm². The inventive chemically amplified resist composition is suited for micropatterning using high-energy radiation such as KrF excimer laser of wavelength 248 nm, ArF excimer laser of wavelength 193 nm, EB, EUV of wavelength 3 to 15 nm, x-ray, soft x-ray, γ-ray or synchrotron radiation.

After the exposure, the resist film may be baked (PEB) on a hotplate preferably at 60 to 150° C. for 10 seconds to 30 minutes, more preferably at 80 to 120° C. for 30 seconds to 20 minutes.

After the exposure or PEB, the resist film is developed with a developer in the form of an aqueous base solution for 3 seconds to 3 minutes, preferably 5 seconds to 2 minutes by conventional techniques such as dip, puddle and spray techniques. A typical developer is a 0.1 to 10 wt %, preferably 2 to 5 wt % aqueous solution of tetramethylammonium hydroxide (TMAH), tetraethylammonium hydroxide (TEAH), tetrapropylammonium hydroxide (TPAH), or tetrabutylammonium hydroxide (TBAH). The resist film in the exposed area is dissolved in the developer whereas the resist film in the unexposed area is not dissolved. In this way, the desired positive pattern is formed on the substrate.

In an alternative embodiment, a negative pattern may be formed via organic solvent development. Examples of the developer used herein include 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, benzyl formate, phenylethyl formate, methyl 3-phenylpropionate, benzyl propionate, ethyl phenylacetate, and 2-phenylethyl acetate. The organic solvents may be used alone or in admixture.

At the end of development, the resist film may be rinsed. As the rinsing liquid, a solvent which is miscible with the developer and does not dissolve the resist film is preferred. Suitable solvents include alcohols of 3 to 10 carbon atoms, ether compounds of 8 to 12 carbon atoms, alkanes, alkenes, and alkynes of 6 to 12 carbon atoms, and aromatic solvents.

Specifically, suitable alcohols of 3 to 10 carbon atoms include n-propyl alcohol, isopropyl alcohol, 1-butyl alcohol, 2-butyl alcohol, isobutyl alcohol, tert-butyl alcohol, 1-pentanol, 2-pentanol, 3-pentanol, tert-pentyl alcohol, neopentyl alcohol, 2-methyl-1-butanol, 3-methyl-1-butanol, 3-methyl-3-pentanol, cyclopentanol, 1-hexanol, 2-hexanol, 3-hexanol, 2,3-dimethyl-2-butanol, 3,3-dimethyl-1-butanol, 3,3-dimethyl-2-butanol, 2-ethyl-1-butanol, 2-methyl-1-pentanol, 2-methyl-2-pentanol, 2-methyl-3-pentanol, 3-methyl-1-pentanol, 3-methyl-2-pentanol, 3-methyl-3-pentanol, 4-methyl-1-pentanol, 4-methyl-2-pentanol, 4-methyl-3-pentanol, cyclohexanol, and 1-octanol.

Suitable ether compounds of 8 to 12 carbon atoms include di-n-butyl ether, diisobutyl ether, di-sec-butyl ether, di-n-pentyl ether, diisopentyl ether, di-sec-pentyl ether, di-tert-pentyl ether, and di-n-hexyl ether.

Suitable alkenes of 6 to 12 carbon atoms include hexane, heptane, octane, nonane, decane, undecane, dodecane, methylcyclopentane, dimethylcyclopentane, cyclohexane, methylcyclohexane, dimethylcyclohexane, cycloheptane, cyclooctane, and cyclononane.

Suitable alkynes of 6 to 12 carbon atoms include hexene, heptene, octene, cyclohexene, methylcyclohexene, dimethylcyclohexene, cycloheptene, and cyclooctene. Suitable alkynes of 6 to 12 carbon atoms include hexyne, heptyne, and octyne.

Suitable aromatic solvents include toluene, xylene, ethylbenzene, isopropylbenzene, tert-butylbenzene and mesitylene.

Rinsing is effective for minimizing the risks of resist pattern collapse and defect formation. However, rinsing is not essential. If rinsing is omitted, the amount of solvent used may be reduced.

A hole or trench pattern after development may be shrunk by the thermal flow, RELACS® or DSA process. A hole pattern is shrunk by coating a shrink agent thereto, and baking such that the shrink agent may undergo crosslinking at the resist surface as a result of the acid catalyst diffusing from the resist layer during bake, and the shrink agent may attach to the sidewall of the hole pattern. The bake is preferably at a temperature of 70 to 180° C., more preferably 80 to 170° C., for a time of 10 to 300 seconds. The extra shrink agent is stripped and the hole pattern is shrunk.

### EXAMPLES

Synthesis Examples, Examples, and Comparative Examples of the invention are given below by way of illustration and not by way of limitation. The apparatuses used are as follows.

MALDI TOF-MS: S3000 manufactured by JEOL Ltd.

### [1] Synthesis of Sulfonium Salt Monomers

### Example 1-1

### Synthesis of Sulfonium Salt Monomer a-1

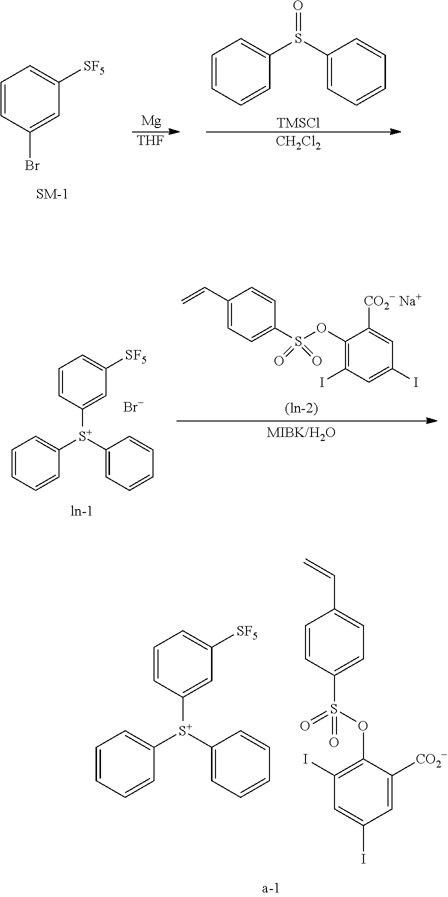

### (1) Synthesis of Intermediate In-1

In nitrogen atmosphere, a Grignard reagent was prepared using 4.5 g of magnesium, 100 g of THF, and 51.0 g of intermediate In-1. The reaction mixture was cooled to 10° C. or lower, and a solution of 12.1 g of diphenyl sulfoxide and 50 g of methylene chloride was added. After addition, 19.6 g of chlorotrimethylsilane was added dropwise while the reaction mixture was kept below 20° C. After dropwise addition, the reaction mixture was aged at a temperature of 20° C. or lower for 2 hours. After aging, the reaction mixture was cooled, after which an aqueous solution of 15 g of ammonium chloride, 15 g of 20 wt % hydrochloric acid and 100 g of water was added dropwise to quench the reaction. Subsequently, 50 g of methanol and 200 g of diisopropyl ether were added, and the aqueous layer was taken out. The aqueous layer was washed twice with 200 g of hexane. After washing, 100 g of methylene chloride was added, and the end product was extracted. This was followed by ordinary aqueous work-up, and solvent distillation. Intermediate In-2 was obtained as colorless oily matter (amount 24.5 g, yield 87%).

### (2) Synthesis of Onium Salt Monomer a-1

In nitrogen atmosphere, a reactor was charged with 24.5 g of Intermediate In-1, 22.0 g of Intermediate In-2, 150 g of methylene chloride, and 70 g of water. The mixture was stirred for 30 minutes at room temperature. The organic layer was taken out, washed with water, and concentrated under reduced pressure. The residue was washed with diisopropyl ether, and concentrated to obtain PAG-1 as oily matter (amount 33.8 g, yield 94%).

Sulfonium salt monomer a-1 was analyzed by TOF-MS, with the data shown below.

MALDI TOF-MS:
POSITIVE M+389 (corresponding to C₁₈H₁₄F₅S₂⁺)
NEGATIVE M-555 (corresponding to C₁₅H₉I₂O₅S⁻)

### Examples 1-2 to 1-7

### Synthesis of Sulfonium Salt Monomers a-2 to a-7

Sulfonium Salt Monomers a-2 to a-7 of the following formulae were synthesized using the corresponding reactants and well-known organic chemistry reaction.

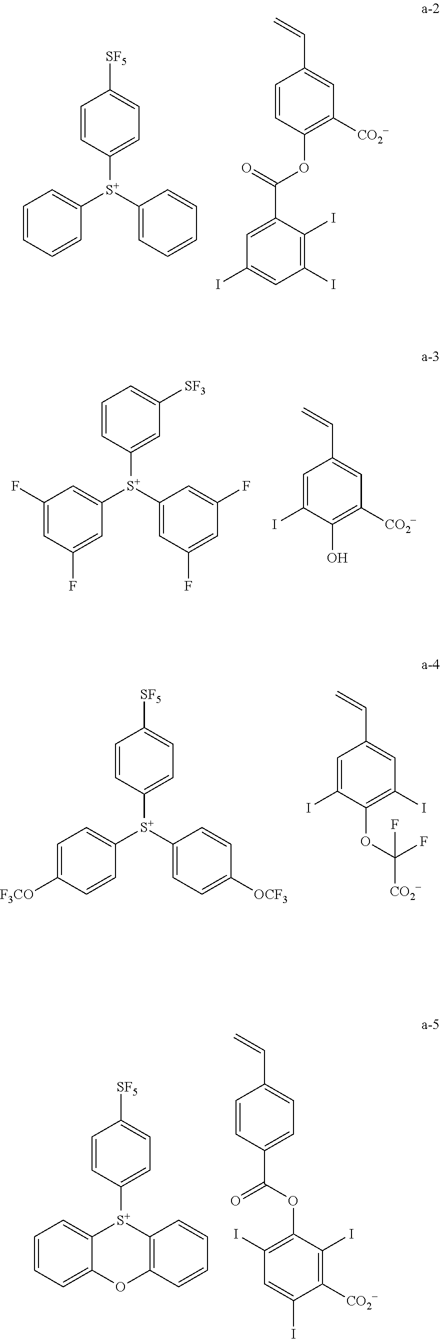

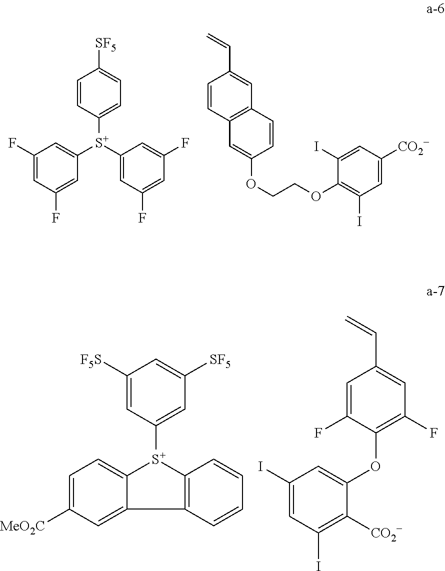

### Comparative Examples 1-1 to 1-5

Synthesis of Comparative Onium Salt Monomers ca-1 to ca-5

Comparative onium salt monomers ca-1 to ca-5 of the following formulae were synthesized using the corresponding reactants and well-known organic chemistry reaction.

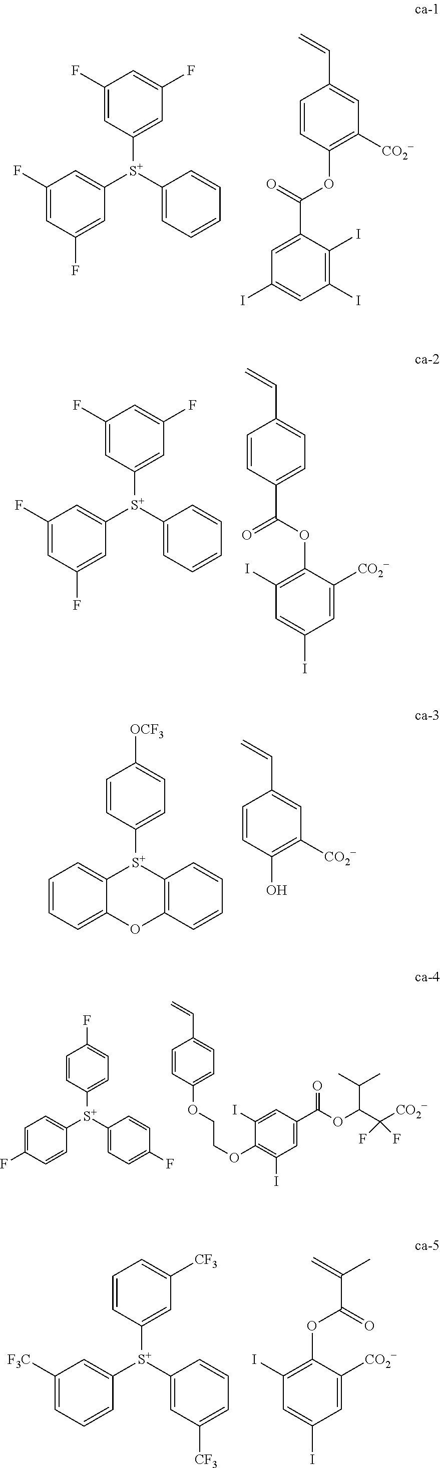

### [2] Synthesis of Base Polymer

Monomers a-1 to a-7, comparative monomers ca-1 to ca-5, and the monomers shown below were used in the synthesis of base polymers.

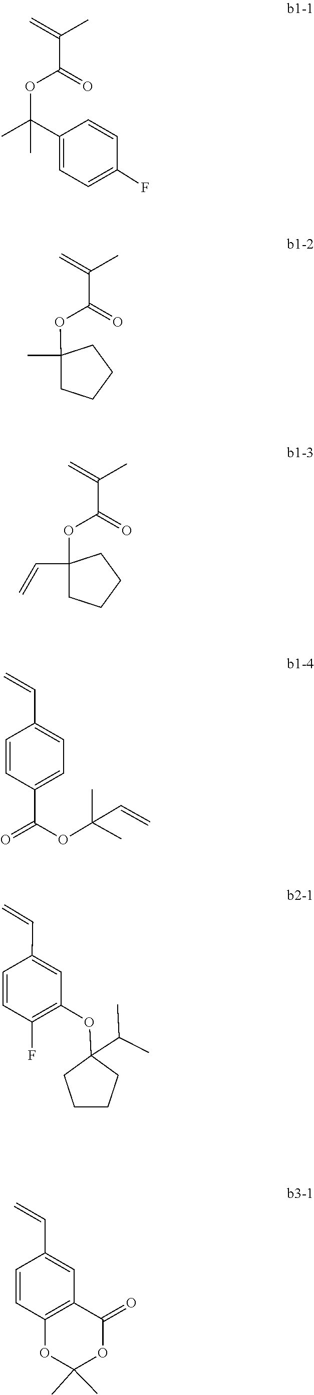

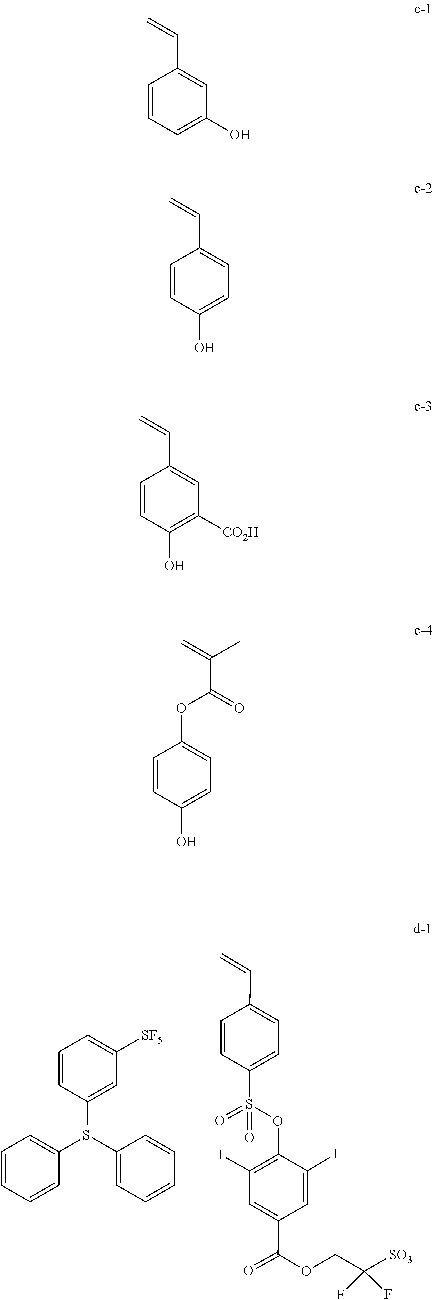

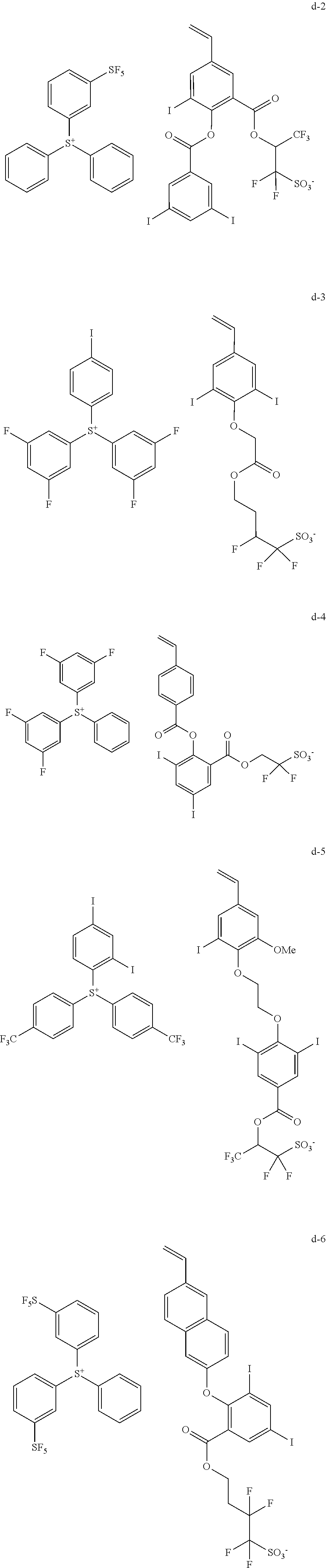

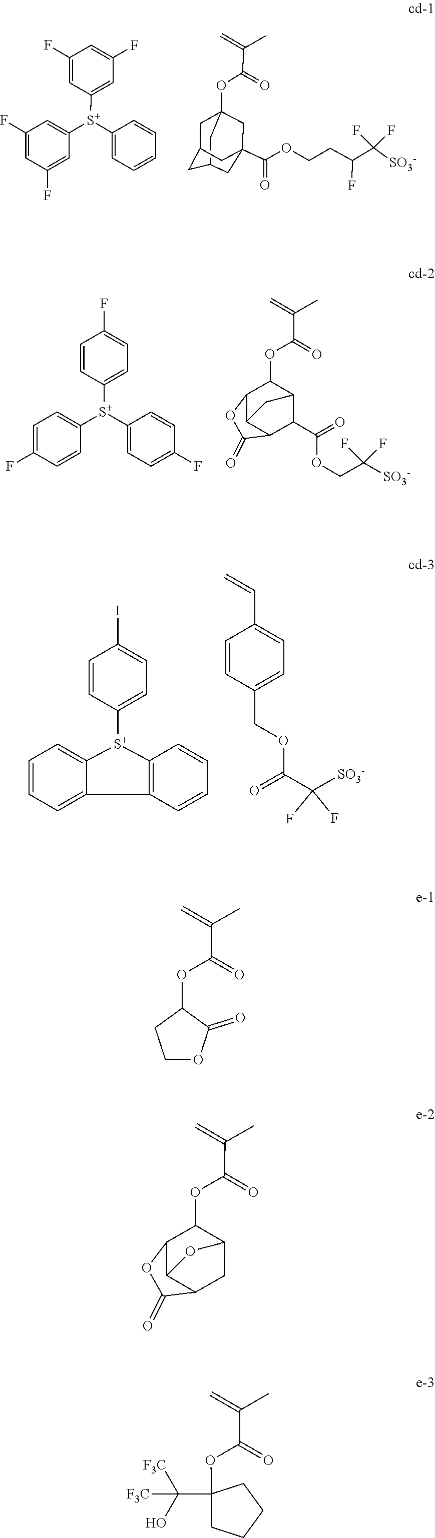

### Example 2-1

### Synthesis of Polymer P-1

A flask under nitrogen atmosphere was charged with 13.7 g of Monomer a-1, 30.0 g of Monomer b1-1, 9.7 g of Monomer c-1, 46.8 g of Monomer d-1, 3.09 g of V-601 (manufactured by Fujifilm Wako Pure Chemical Corp.), and 139 g of MEK to prepare a monomer/initiator solution. Another flask under nitrogen atmosphere was charged with 46 g of MEK, which was heated to 80° C. with stirring. The monomer/initiator solution was added dropwise to the MEK over 4 hours. At the end of dropwise addition, the polymerization solution was continuously stirred for 2 hours while maintaining the temperature of 80° C. The polymerization solution was cooled to room temperature. The obtained polymerization solution was added dropwise to 3,000 g of hexane with vigorous stirring. The precipitate was collected by filtration. The precipitate was washed twice with 600 g of hexane and vacuum dried at 50° C. for 20 hours to obtain Polymer P-1 as white powder (amount 96.1 g, yield 96%). Polymer P-1 had a Mw of 10,300, and a Mw/Mn of 1.61. It is noted that Mw is measured by GPC versus polystyrene standards using DMF solvent.

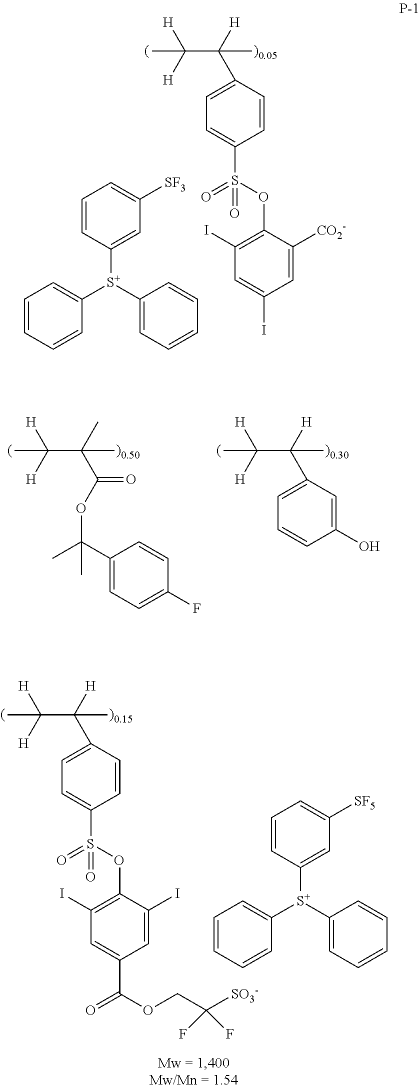

### Examples 2-2 to 2-50 and Comparative Examples 2-1 to 2-38

### Synthesis of Polymers P-2 to P-50 and Comparative Polymers CP-1 to CP-38

Polymers shown in Tables 1 to 3 were synthesized by the same procedure as in Synthesis Example 2-1 except that the type and amount (blending ratio) of monomers were changed.

TABLE 1

| | | Incorpo- | | Incorpo- | | Incorpo- | | Incorpo- | | Incorpo- | | |
|---|---|---|---|---|---|---|---|---|---|---|---|---|
| | | ration | | ration | | ration | | ration | | ration | | |
| | | ratio | | ratio | | ratio | | ratio | | ratio | | |
| Polymer | Unit 1 | (mol %) | Unit 2 | (mol %) | Unit 3 | (mol %) | Unit 4 | (mol %) | Unit 5 | (mol %) | Mw | Mw/Mn |
| P-1 | a-1 | 5 | b1-1 | 50 | c-1 | 30 | d-1 | 15 | — | — | 10,300 | 1.61 |
| P-2 | a-2 | 5 | b1-1 | 50 | c-1 | 30 | d-1 | 15 | — | — | 10,600 | 1.62 |
| P-3 | a-3 | 5 | b1-1 | 50 | c-1 | 30 | d-1 | 15 | — | — | 10,200 | 1.61 |
| P-4 | a-4 | 5 | b1-1 | 50 | c-1 | 30 | d-1 | 15 | — | — | 10,700 | 1.61 |
| P-5 | a-5 | 5 | b1-1 | 50 | c-1 | 30 | d-1 | 15 | — | — | 10,400 | 1.62 |
| P-6 | a-6 | 5 | b1-1 | 50 | c-1 | 30 | d-1 | 15 | — | — | 10,500 | 1.63 |
| P-7 | a-7 | 5 | b1-1 | 50 | c-1 | 30 | d-1 | 15 | — | — | 10,000 | 1.62 |
| P-8 | a-1 | 5 | b1-2 | 50 | c-1 | 30 | d-1 | 15 | — | — | 10,100 | 1.61 |
| P-9 | a-2 | 5 | b1-2 | 50 | c-1 | 30 | d-1 | 15 | — | — | 10,200 | 1.60 |
| P-10 | a-3 | 5 | b1-2 | 50 | c-1 | 30 | d-1 | 15 | — | — | 10,200 | 1.61 |
| P-11 | a-4 | 5 | b1-2 | 50 | c-1 | 30 | d-1 | 15 | — | — | 10,500 | 1.63 |
| P-12 | a-5 | 5 | b1-2 | 50 | c-1 | 30 | d-1 | 15 | — | — | 10,200 | 1.64 |
| P-13 | a-6 | 5 | b1-2 | 50 | c-1 | 30 | d-1 | 15 | — | — | 10,100 | 1.61 |
| P-14 | a-7 | 5 | b1-2 | 50 | c-1 | 30 | d-1 | 15 | — | — | 10,700 | 1.62 |
| P-15 | a-1 | 5 | b1-3 | 50 | c-1 | 30 | d-1 | 15 | — | — | 10,200 | 1.61 |
| P-16 | a-2 | 5 | b1-3 | 50 | c-1 | 30 | d-1 | 15 | — | — | 10,100 | 1.62 |
| P-17 | a-3 | 5 | b1-3 | 50 | c-1 | 30 | d-1 | 15 | — | — | 10,000 | 1.64 |
| P-18 | a-4 | 5 | b1-3 | 50 | c-1 | 30 | d-1 | 15 | — | — | 10,400 | 1.62 |
| P-19 | a-5 | 5 | b1-3 | 50 | c-1 | 30 | d-1 | 15 | — | — | 10,200 | 1.61 |
| P-20 | a-6 | 5 | b1-3 | 50 | c-1 | 30 | d-1 | 15 | — | — | 10,500 | 1.62 |
| P-21 | a-7 | 5 | b1-3 | 50 | c-1 | 30 | d-1 | 15 | — | — | 10,700 | 1.64 |
| P-22 | a-1 | 5 | b1-4 | 50 | c-1 | 30 | d-1 | 15 | — | — | 10,300 | 1.62 |
| P-23 | a-2 | 5 | b1-4 | 50 | c-1 | 30 | d-1 | 15 | — | — | 10,400 | 1.61 |
| P-24 | a-3 | 5 | b1-4 | 50 | c-1 | 30 | d-1 | 15 | — | — | 10,100 | 1.62 |
| P-25 | a-4 | 5 | b1-4 | 50 | c-1 | 30 | d-1 | 15 | — | — | 10,200 | 1.61 |
| P-26 | a-5 | 5 | b1-4 | 50 | c-1 | 30 | d-1 | 15 | — | — | 10,400 | 1.61 |
| P-27 | a-6 | 5 | b1-4 | 50 | c-1 | 30 | d-1 | 15 | — | — | 10,600 | 1.62 |
| P-28 | a-7 | 5 | b1-4 | 50 | c-1 | 30 | d-1 | 15 | — | — | 10,400 | 1.63 |
| P-29 | a-1 | 5 | b1-1 | 25 | b2-1 | 25 | c-2 | 30 | d-1 | 15 | 10,200 | 1.61 |
| P-30 | a-1 | 5 | b1-2 | 25 | b2-1 | 25 | c-2 | 30 | d-1 | 15 | 10,400 | 1.62 |

TABLE 2

| | | Incorpo- | | Incorpo- | | Incorpo- | | Incorpo- | | Incorpo- | | |
|---|---|---|---|---|---|---|---|---|---|---|---|---|
| | | ration | | ration | | ration | | ration | | ration | | |
| | | ratio | | ratio | | ratio | | ratio | | ratio | | |
| Polymer | Unit 1 | (mol %) | Unit 2 | (mol %) | Unit 3 | (mol %) | Unit 4 | (mol %) | Unit 5 | (mol %) | Mw | Mw/Mn |
| P-31 | a-1 | 5 | b1-3 | 25 | b2-1 | 25 | c-2 | 30 | d-1 | 8 | 10,400 | 1.63 |
| P-32 | a-1 | 5 | b1-3 | 30 | b2-1 | 25 | c-3 | 25 | d-1 | 15 | 10,400 | 1.63 |
| P-33 | a-1 | 5 | b1-1 | 25 | b2-1 | 25 | c-4 | 30 | d-2 | 15 | 10,400 | 1.62 |
| P-34 | a-2 | 5 | b1-1 | 30 | b3-1 | 15 | c-1 | 30 | d-2 | 15 | 10,400 | 1.61 |
| P-35 | a-2 | 5 | b1-1 | 25 | b1-4 | 25 | c-2 | 30 | d-1 | 15 | 10,300 | 1.62 |
| P-36 | a-1 | 5 | b1-1 | 50 | c-1 | 30 | d-2 | 15 | — | — | 10,500 | 1.62 |
| P-37 | a-1 | 5 | b1-1 | 50 | c-1 | 30 | d-3 | 15 | — | — | 10,300 | 1.61 |
| P-38 | a-2 | 5 | b1-1 | 50 | c-1 | 30 | d-4 | 15 | — | — | 10,400 | 1.62 |
| P-39 | a-3 | 5 | b1-1 | 50 | c-1 | 30 | d-5 | 15 | — | — | 10,700 | 1.61 |
| P-40 | a-4 | 5 | b1-1 | 50 | c-1 | 30 | d-6 | 15 | — | — | 10,400 | 1.60 |
| P-41 | a-2 | 5 | b1-1 | 45 | c-2 | 25 | d-1 | 15 | e-1 | 10 | 10,400 | 1.62 |
| P-42 | a-3 | 5 | b1-3 | 45 | c-4 | 25 | d-2 | 15 | e-2 | 10 | 10,400 | 1.63 |
| P-43 | a-4 | 5 | b1-3 | 45 | c-2 | 25 | d-3 | 10 | e-3 | 5 | 10,700 | 1.61 |
| P-44 | a-1 | 7 | b1-1 | 58 | c-1 | 35 | — | — | — | — | 10,400 | 1.62 |
| P-45 | a-2 | 7 | b1-1 | 58 | c-1 | 35 | — | — | — | — | 10,300 | 1.61 |
| P-46 | a-3 | 7 | b1-2 | 58 | c-2 | 35 | — | — | — | — | 10,400 | 1.63 |
| P-47 | a-4 | 7 | b1-3 | 58 | c-3 | 35 | — | — | — | — | 10,700 | 1.62 |
| P-48 | a-5 | 7 | b1-4 | 58 | c-4 | 35 | — | — | — | — | 10,500 | 1.61 |
| P-49 | a-6 | 7 | b1-1 | 29 | b2-1 | 29 | c-1 | 35 | — | — | 10,400 | 1.62 |
| P-50 | a-7 | 7 | b1-1 | 29 | b3-1 | 29 | c-2 | 35 | — | — | 10,300 | 1.61 |

TABLE 3

| | | Incorpo- | | Incorpo- | | Incorpo- | | Incorpo- | | Incorpo- | | |
|---|---|---|---|---|---|---|---|---|---|---|---|---|
| | | ration | | ration | | ration | | ration | | ration | | |
| | | ratio | | ratio | | ratio | | ratio | | ratio | | |
| Polymer | Unit 1 | (mol %) | Unit 2 | (mol %) | Unit 3 | (mol %) | Unit 4 | (mol %) | Unit 5 | (mol %) | Mw | Mw/Mn |
| CP-1 | ca-1 | 5 | b1-1 | 50 | c-1 | 30 | d-1 | 15 | — | — | 10,200 | 1.61 |
| CP-2 | ca-2 | 5 | b1-1 | 50 | c-1 | 30 | d-1 | 15 | — | — | 10,200 | 1.62 |
| CP-3 | ca-3 | 5 | b1-1 | 50 | c-1 | 30 | d-1 | 15 | — | — | 10,500 | 1.64 |
| CP-4 | ca-4 | 5 | b1-1 | 50 | c-1 | 30 | d-1 | 15 | — | — | 10,300 | 1.63 |
| CP-5 | ca-5 | 5 | b1-1 | 50 | c-1 | 30 | d-1 | 15 | — | — | 10,400 | 1.62 |
| CP-6 | ca-1 | 5 | b1-2 | 50 | c-1 | 30 | d-1 | 15 | — | — | 10,300 | 1.61 |
| CP-7 | ca-2 | 5 | b1-2 | 50 | c-1 | 30 | d-1 | 15 | — | — | 10,200 | 1.62 |
| CP-8 | ca-3 | 5 | b1-2 | 50 | c-1 | 30 | d-1 | 15 | — | — | 10,600 | 1.60 |
| CP-9 | ca-4 | 5 | b1-2 | 50 | c-1 | 30 | d-1 | 15 | — | — | 10,800 | 1.61 |
| CP-10 | ca-5 | 5 | b1-2 | 50 | c-1 | 30 | d-1 | 15 | — | — | 10,500 | 1.62 |
| CP-11 | ca-1 | 5 | b1-3 | 50 | c-1 | 30 | d-1 | 15 | — | — | 10,400 | 1.63 |
| CP-12 | ca-2 | 5 | b1-3 | 50 | c-1 | 30 | d-1 | 15 | — | — | 10,300 | 1.61 |
| CP-13 | ca-3 | 5 | b1-3 | 50 | c-1 | 30 | d-1 | 15 | — | — | 10,600 | 1.62 |
| CP-14 | ca-4 | 5 | b1-3 | 50 | c-1 | 30 | d-1 | 15 | — | — | 10,100 | 1.63 |
| CP-15 | ca-5 | 5 | b1-3 | 50 | c-1 | 30 | d-1 | 15 | — | — | 10,200 | 1.61 |
| CP-16 | ca-1 | 5 | b1-4 | 50 | c-1 | 30 | d-1 | 15 | — | — | 10,400 | 1.63 |
| CP-17 | ca-2 | 5 | b1-4 | 50 | c-1 | 30 | d-1 | 15 | — | — | 10,300 | 1.65 |
| CP-18 | ca-3 | 5 | b1-4 | 50 | c-1 | 30 | d-1 | 15 | — | — | 10,400 | 1.62 |
| CP-19 | ca-4 | 5 | b1-4 | 50 | c-1 | 30 | d-1 | 15 | — | — | 10,600 | 1.62 |
| CP-20 | ca-5 | 5 | b1-4 | 50 | c-1 | 30 | d-1 | 15 | — | — | 10,100 | 1.61 |
| CP-21 | ca-1 | 5 | b1-1 | 25 | b2-1 | 25 | c-2 | 30 | d-1 | 15 | 10,200 | 1.62 |
| CP-22 | ca-2 | 5 | b1-2 | 25 | b2-1 | 25 | c-2 | 30 | d-2 | 15 | 10,000 | 1.63 |
| CP-23 | ca-4 | 5 | b1-3 | 25 | b2-1 | 25 | c-2 | 30 | d-4 | 8 | 10,300 | 1.64 |
| CP-24 | ca-1 | 5 | b1-1 | 25 | b2-1 | 25 | c-2 | 30 | cd-1 | 15 | 10,200 | 1.62 |
| CP-25 | ca-2 | 5 | b1-2 | 25 | b2-1 | 25 | c-2 | 30 | cd-2 | 15 | 10,600 | 1.61 |
| CP-26 | ca-5 | 5 | b1-3 | 25 | b2-1 | 25 | c-2 | 30 | cd-3 | 15 | 10,300 | 1.62 |
| CP-27 | ca-1 | 5 | b1-1 | 50 | c-1 | 30 | cd-1 | 15 | — | — | 10,100 | 1.63 |
| CP-28 | ca-1 | 5 | b1-1 | 50 | c-1 | 30 | cd-2 | 15 | — | — | 10,300 | 1.62 |
| CP-29 | ca-2 | 5 | b1-1 | 50 | c-1 | 30 | cd-3 | 15 | — | — | 10,200 | 1.61 |
| CP-30 | ca-1 | 5 | b1-1 | 45 | c-2 | 25 | d-1 | 15 | e-1 | 10 | 10,400 | 1.63 |
| CP-31 | ca-2 | 5 | b1-3 | 45 | c-4 | 25 | cd-1 | 15 | e-2 | 10 | 10,500 | 1.63 |
| CP-32 | ca-4 | 5 | b1-3 | 45 | c-2 | 25 | cd-2 | 10 | e-3 | 5 | 10,300 | 1.62 |
| CP-33 | ca-1 | 7 | b1-1 | 58 | c-1 | 35 | — | — | — | — | 10,200 | 1.61 |
| CP-34 | ca-2 | 7 | b1-1 | 58 | c-2 | 35 | — | — | — | — | 10,200 | 1.63 |
| CP-35 | ca-4 | 7 | b1-2 | 58 | c-2 | 35 | — | — | — | — | 10,400 | 1.62 |
| CP-36 | ca-1 | 7 | b1-1 | 29 | b2-1 | 29 | c-1 | 35 | — | — | 10,600 | 1.65 |
| CP-37 | b1-1 | 55 | c-1 | 30 | cd-1 | 15 | — | — | — | — | 10,300 | 1.61 |
| CP-38 | b1-2 | 50 | c-2 | 50 | — | — | — | — | — | — | 10,100 | 1.64 |

### [3] Preparation of Chemically Amplified Resist Composition

### Examples 3-1 to 3-53 and Comparative Examples 3-1 to 3-40

A chemically amplified resist composition (R-1 to R-53, CR-1 to CR-40) was prepared by dissolving an inventive base polymer (P-1 to P-50) or comparative base polymer (CP-1 to CP-38), acid generator (PAG-1, PAG-2), and quencher (SQ-1, AQ-1) in a solvent containing 0.01 wt % of surfactant FC-4430 (3M) in accordance with the formulation shown in Tables 4 to 6, and filtering the solution through a Teflon® filter with a pore size of 0.2 μm.

TABLE 4

| | | | Base | | Acid | | | |
|---|---|---|---|---|---|---|---|---|
| | | Resist | polymer | Quencher | generator | Solvent 1 | Solvent 2 | Solvent 3 |
| | | composition | (pbw) | (pbw) | (pbw) | (pbw) | (pbw) | (pbw) |
| Example | 3-1 | R-1 | P-1 (80) | — | — | PGMEA (2000) | EL (2500) | DAA (500) |
| | 3-2 | R-2 | P-2 (80) | — | — | PGMEA (2000) | EL (2500) | DAA (500) |
| | 3-3 | R-3 | P-3 (80) | — | — | PGMEA (2000) | EL (2500) | DAA (500) |
| | 3-4 | R-4 | P-4 (80) | — | — | PGMEA (2000) | EL (2500) | DAA (500) |
| | 3-5 | R-5 | P-5 (80) | — | — | PGMEA (2000) | EL (2500) | DAA (500) |
| | 3-6 | R-6 | P-6 (80) | — | — | PGMEA (2000) | EL (2500) | DAA (500) |
| | 3-7 | R-7 | P-7 (80) | — | — | PGMEA (2000) | EL (2500) | DAA (500) |
| | 3-8 | R-8 | P-8 (80) | — | — | PGMEA (2000) | EL (2500) | DAA (500) |
| | 3-9 | R-9 | P-9 (80) | — | — | PGMEA (2000) | EL (2500) | DAA (500) |
| | 3-10 | R-10 | P-10 (80) | — | — | PGMEA (2000) | EL (2500) | DAA (500) |
| | 3-11 | R-11 | P-11 (80) | — | — | PGMEA (2000) | EL (2500) | DAA (500) |
| | 3-12 | R-12 | P-12 (80) | — | — | PGMEA (2000) | EL (2500) | DAA (500) |
| | 3-13 | R-13 | P-13 (80) | — | — | PGMEA (2000) | EL (2500) | DAA (500) |
| | 3-14 | R-14 | P-14 (80) | — | — | PGMEA (2000) | EL (2500) | DAA (500) |
| | 3-15 | R-15 | P-15 (80) | — | — | PGMEA (2000) | EL (2500) | DAA (500) |
| | 3-16 | R-16 | P-16 (80) | — | — | PGMEA (2000) | EL (2500) | DAA (500) |
| | 3-17 | R-17 | P-17 (80) | — | — | PGMEA (2000) | EL (2500) | DAA (500) |
| | 3-18 | R-18 | P-18 (80) | — | — | PGMEA (2000) | EL (2500) | DAA (500) |
| | 3-19 | R-19 | P-19 (80) | — | — | PGMEA (2000) | EL (2500) | DAA (500) |
| | 3-20 | R-20 | P-20 (80) | — | — | PGMEA (2000) | EL (2500) | DAA (500) |
| | 3-21 | R-21 | P-21 (80) | — | — | PGMEA (2000) | EL (2500) | DAA (500) |
| | 3-22 | R-22 | P-22 (80) | — | — | PGMEA (2000) | EL (2500) | DAA (500) |
| | 3-23 | R-23 | P-23 (80) | — | — | PGMEA (2000) | EL (2500) | DAA (500) |
| | 3-24 | R-24 | P-24 (80) | — | — | PGMEA (2000) | EL (2500) | DAA (500) |
| | 3-25 | R-25 | P-25 (80) | — | — | PGMEA (2000) | EL (2500) | DAA (500) |
| | 3-26 | R-26 | P-26 (80) | — | — | PGMEA (2000) | EL (2500) | DAA (500) |
| | 3-27 | R-27 | P-27 (80) | — | — | PGMEA (2000) | EL (2500) | DAA (500) |
| | 3-28 | R-28 | P-28 (80) | — | — | PGMEA (2000) | EL (2500) | DAA (500) |
| | 3-29 | R-29 | P-29 (80) | — | — | PGMEA (2000) | EL (2500) | DAA (500) |
| | 3-30 | R-30 | P-30 (80) | — | — | PGMEA (2000) | EL (2500) | DAA (500) |

TABLE 5

| | | | Base | | Acid | | | |
|---|---|---|---|---|---|---|---|---|
| | | Resist | polymer | Quencher | generator | Solvent 1 | Solvent 2 | Solvent 3 |
| | | composition | (pbw) | (pbw) | (pbw) | (pbw) | (pbw) | (pbw) |
| Example | 3-31 | R-31 | P-31 (80) | — | PAG-1 (10) | PGMEA (2000) | EL (2500) | DAA (500) |
| | 3-32 | R-32 | P-32 (80) | — | — | PGMEA (2000) | EL (2500) | DAA (500) |
| | 3-33 | R-33 | P-33 (80) | — | — | PGMEA (2000) | EL (2500) | DAA (500) |
| | 3-34 | R-34 | P-34 (80) | — | — | PGMEA (2000) | EL (2500) | DAA (500) |
| | 3-35 | R-35 | P-35 (80) | — | — | PGMEA (2000) | EL (2500) | DAA (500) |
| | 3-36 | R-36 | P-36 (80) | — | — | PGMEA (2000) | EL (2500) | DAA (500) |
| | 3-37 | R-37 | P-37 (80) | — | — | PGMEA (2000) | EL (2500) | DAA (500) |
| | 3-38 | R-38 | P-38 (80) | — | — | PGMEA (2000) | EL (2500) | DAA (500) |
| | 3-39 | R-39 | P-39 (80) | — | — | PGMEA (2000) | EL (2500) | DAA (500) |
| | 3-40 | R-40 | P-40 (80) | — | — | PGMEA (2000) | EL (2500) | DAA (500) |
| | 3-41 | R-41 | P-41 (80) | — | — | PGMEA (2000) | EL (2500) | DAA (500) |
| | 3-42 | R-42 | P-42 (80) | — | — | PGMEA (2000) | EL (2500) | DAA (500) |
| | 3-43 | R-43 | P-43 (80) | — | PAG-2 (10) | PGMEA (2000) | EL (2500) | DAA (500) |
| | 3-44 | R-44 | P-44 (80) | — | PAG-1 (10) | PGMEA (2000) | EL (2500) | DAA (500) |
| | 3-45 | R-45 | P-45 (80) | — | PAG-1 (10) | PGMEA (2000) | EL (2500) | DAA (500) |
| | 3-46 | R-46 | P-46 (80) | — | PAG-1 (10) | PGMEA (2000) | EL (2500) | DAA (500) |
| | 3-47 | R-47 | P-47 (80) | — | PAG-2 (10) | PGMEA (2000) | EL (2500) | DAA (500) |
| | 3-48 | R-48 | P-48 (80) | — | PAG-1 (10) | PGMEA (2000) | EL (2500) | DAA (500) |
| | 3-49 | R-49 | P-49 (80) | — | PAG-2 (10) | PGMEA (2000) | EL (2500) | DAA (500) |
| | 3-50 | R-50 | P-50 (80) | — | PAG-1 (10) | PGMEA (2000) | EL (2500) | DAA (500) |
| | 3-51 | R-51 | P-1 (80) | SQ-1 (4) | — | PGMEA (2000) | EL (2500) | DAA (500) |
| | 3-52 | R-52 | P-1 (80) | AQ-1 (4) | — | PGMEA (2000) | EL (2500) | DAA (500) |
| | 3-53 | R-53 | P-1 (80) | SQ-1 (2) | — | PGMEA (2000) | EL (2500) | DAA (500) |
| | | | | AQ-1 (2) | | | | |

TABLE 6

| | | | Base | | Acid | | | |
|---|---|---|---|---|---|---|---|---|
| | | Resist | polymer | Quencher | generator | Solvent 1 | Solvent 2 | Solvent 3 |
| | | composition | (pbw) | (pbw) | (pbw) | (pbw) | (pbw) | (pbw) |
| Comparative | 3-1 | CR-1 | CP-1 (80) | — | — | PGMEA (2000) | EL (2500) | DAA (500) |
| Example | 3-2 | CR-2 | CP-2 (80) | — | — | PGMEA (2000) | EL (2500) | DAA (500) |
| | 3-3 | CR-3 | CP-3 (80) | — | — | PGMEA (2000) | EL (2500) | DAA (500) |
| | 3-4 | CR-4 | CP-4 (80) | — | — | PGMEA (2000) | EL (2500) | DAA (500) |
| | 3-5 | CR-5 | CP-5 (80) | — | — | PGMEA (2000) | EL (2500) | DAA (500) |
| | 3-6 | CR-6 | CP-6 (80) | — | — | PGMEA (2000) | EL (2500) | DAA (500) |
| | 3-7 | CR-7 | CP-7 (80) | — | — | PGMEA (2000) | EL (2500) | DAA (500) |
| | 3-8 | CR-8 | CP-8 (80) | — | — | PGMEA (2000) | EL (2500) | DAA (500) |
| | 3-9 | CR-9 | CP-9 (80) | — | — | PGMEA (2000) | EL (2500) | DAA (500) |
| | 3-10 | CR-10 | CP-10 (80) | — | — | PGMEA (2000) | EL (2500) | DAA (500) |
| | 3-11 | CR-11 | CP-11 (80) | — | — | PGMEA (2000) | EL (2500) | DAA (500) |
| | 3-12 | CR-12 | CP-12 (80) | — | — | PGMEA (2000) | EL (2500) | DAA (500) |
| | 3-13 | CR-13 | CP-13 (80) | — | — | PGMEA (2000) | EL (2500) | DAA (500) |
| | 3-14 | CR-14 | CP-14 (80) | — | — | PGMEA (2000) | EL (2500) | DAA (500) |
| | 3-15 | CR-15 | CP-15 (80) | — | — | PGMEA (2000) | EL (2500) | DAA (500) |
| | 3-16 | CR-16 | CP-16 (80) | — | — | PGMEA (2000) | EL (2500) | DAA (500) |
| | 3-17 | CR-17 | CP-17 (80) | — | — | PGMEA (2000) | EL (2500) | DAA (500) |
| | 3-18 | CR-18 | CP-18 (80) | — | — | PGMEA (2000) | EL (2500) | DAA (500) |
| | 3-19 | CR-19 | CP-19 (80) | — | — | PGMEA (2000) | EL (2500) | DAA (500) |
| | 3-20 | CR-20 | CP-20 (80) | — | — | PGMEA (2000) | EL (2500) | DAA (500) |
| | 3-21 | CR-21 | CP-21 (80) | — | — | PGMEA (2000) | EL (2500) | DAA (500) |
| | 3-22 | CR-22 | CP-22 (80) | — | — | PGMEA (2000) | EL (2500) | DAA (500) |
| | 3-23 | CR-23 | CP-23 (80) | — | PAG-1 (10) | PGMEA (2000) | EL (2500) | DAA (500) |
| | 3-24 | CR-24 | CP-24 (80) | — | — | PGMEA (2000) | EL (2500) | DAA (500) |
| | 3-25 | CR-25 | CP-25 (80) | — | — | PGMEA (2000) | EL (2500) | DAA (500) |
| | 3-26 | CR-26 | CP-26 (80) | — | — | PGMEA (2000) | EL (2500) | DAA (500) |
| | 3-27 | CR-27 | CP-27 (80) | — | — | PGMEA (2000) | EL (2500) | DAA (500) |
| | 3-28 | CR-28 | CP-28 (80) | — | — | PGMEA (2000) | EL (2500) | DAA (500) |
| | 3-29 | CR-29 | CP-29 (80) | — | — | PGMEA (2000) | EL (2500) | DAA (500) |
| | 3-30 | CR-30 | CP-30 (80) | — | — | PGMEA (2000) | EL (2500) | DAA (500) |
| | 3-31 | CR-31 | CP-31 (80) | — | — | PGMEA (2000) | EL (2500) | DAA (500) |
| | 3-32 | CR-32 | CP-32 (80) | — | PAG-2 (10) | PGMEA (2000) | EL (2500) | DAA (500) |
| | 3-33 | CR-33 | CP-33 (80) | — | PAG-1 (20) | PGMEA (2000) | EL (2500) | DAA (500) |
| | 3-34 | CR-34 | CP-34 (80) | — | PAG-1 (20) | PGMEA (2000) | EL (2500) | DAA (500) |
| | 3-35 | CR-35 | CP-35 (80) | — | PAG-2 (20) | PGMEA (2000) | EL (2500) | DAA (500) |
| | 3-36 | CR-36 | CP-36 (80) | — | PAG-1 (20) | PGMEA (2000) | EL (2500) | DAA (500) |
| | 3-37 | CR-37 | CP-37 (80) | SQ-1 (8) | — | PGMEA (2000) | EL (2500) | DAA (500) |
| | 3-38 | CR-38 | CP-38 (80) | SQ-1 (8) | PAG-1 (20) | PGMEA (2000) | EL (2500) | DAA (500) |
| | 3-39 | CR-39 | CP-38 (80) | AQ-1 (8) | PAG-1 (20) | PGMEA (2000) | EL (2500) | DAA (500) |
| | 3-40 | CR-40 | CP-38 (80) | SQ-1 (4) | PAG-1 (20) | PGMEA (2000) | EL (2500) | DAA (500) |
| | | | | AQ-1 (4) | | | | |

The solvents, quenchers (SQ-1, AQ-1) and acid generators (PAG-1, PAG-2) in Tables 4 to 6 are as identified below.

### Solvent:

PGMEA (propylene glycol monomethyl ether acetate)
EL (ethyl lactate)
DAA (diacetone alcohol)

### Quencher: SQ-1, AQ-1

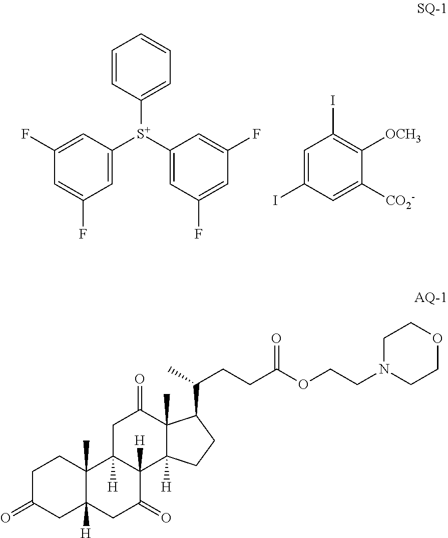

Acid generator: PAG-1, PAG-2

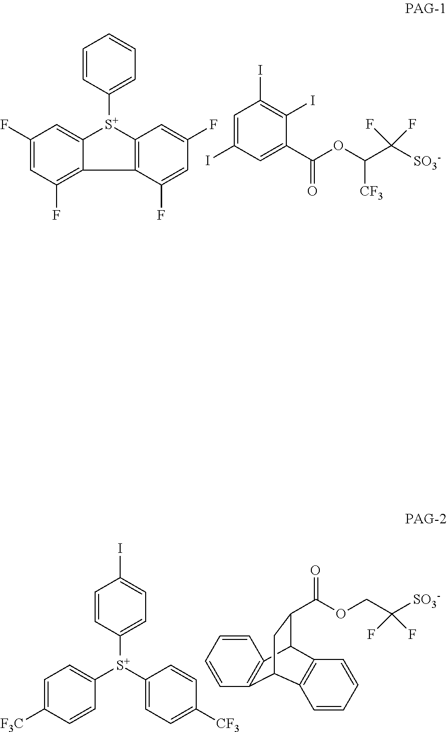

### [4] EUV Lithography Test (1)

### Examples 4-1 to 4-53 and Comparative Examples 4-1 to 4-40

Each of the chemically amplified resist compositions (R-1 to R-53, CR-1 to CR-40) shown in Tables 4 to 6 was spin coated on a silicon substrate having a 20-nm coating of silicon-containing spin-on hard mask SHB-A940 (Shin-Etsu Chemical Co., Ltd., silicon content 43 wt %) and prebaked on a hotplate at 100° C. for 60 seconds to form a resist film of 50 nm thick. Using an EUV scanner NXE3300 manufactured by ASML (NA 0.33, a 0.9/0.6, dipole illumination), the resist film was exposed to EUV through a mask bearing a line-and-space (LS) pattern having a width of 18 nm (on-wafer size) and a pitch of 36 nm while changing the dose at a pitch of 1 mJ/cm² and the focus at a pitch of 0.020 μm. The resist film was baked (PEB) at the temperature shown in Tables 7 to 9 for 60 seconds. This was followed by puddle development in a 2.38 wt % TMAH aqueous solution for 30 seconds, rinsing with a surfactant-containing rinse fluid, and spin drying. A positive LS pattern was obtained.

The obtained LS pattern was observed under CD-SEM (CG6300, Hitachi High-Technologies Corp.) and evaluated for sensitivity, exposure latitude (EL), LWR, depth of focus (DOF), and collapse limit by the following methods. The results are shown in Tables 7 to 9.

### [Evaluation of Sensitivity]

The optimum dose Eop (mJ/cm²) which provided a LS pattern with a line width of 18 nm and a pitch of 36 nm was determined as an index of sensitivity. A smaller value indicates a higher sensitivity.

### [Evaluation of EL]

The exposure dose which provided a LS pattern with a space width of 18 nm±10% (i.e., 16.2 to 19.8 nm) was determined. EL (%) is calculated from the exposure doses according to the following equation: A greater value indicates better performance.

$$EL ( \% ) = ( \vert {E}_{1} - {E}_{2} \vert / Eop ) \times 100$$

wherein E₁ is an optimum exposure dose which provides a LS pattern with a line width of 16.2 nm and a pitch of 36 nm,
E₂ is an optimum exposure dose which provides a LS pattern with a line width of 19.8 nm and a pitch of 36 nm, and
Eop is an optimum exposure dose which provides a LS pattern with a line width of 18 nm and a pitch of 36 nm.

### [Evaluation of LWR]

For the LS pattern formed by exposure at the optimum dose Eop, the line width was measured at 10 longitudinally spaced apart points, from which a 3-fold value (3a) of standard deviation (a) was determined and reported as LWR. A smaller value of 3a indicates a pattern having small roughness and uniform line width.

### [Evaluation of DOF]

As an index of DOF, a range of focus which provided a LS pattern with a size of 18 nm±10% (i.e., 16.2 to 19.8 nm) was determined. A greater value indicates a wider DOF.

### [Evaluation of Collapse Limit of Line Pattern]

For the LS pattern formed by exposure at the dose corresponding to the optimum focus, the line width was measured at 10 longitudinally spaced apart points. The minimum line size above which lines could be resolved without collapse was determined and reported as collapse limit. A smaller value indicates better collapse limit.

TABLE 7

| | | | | Optimal | | | | |
|---|---|---|---|---|---|---|---|---|
| | | Resist | PEB temp. | exposure dose | EL | LWR | DOF | Collapse limit |
| | | composition | (° C.) | (mJ/cm²) | (%) | (nm) | (nm) | (nm) |
| Example | 4-1 | R-1 | 95 | 33 | 18 | 2.4 | 120 | 10.8 |
| | 4-2 | R-2 | 100 | 34 | 19 | 2.5 | 110 | 10.9 |
| | 4-3 | R-3 | 100 | 35 | 17 | 2.6 | 120 | 10.7 |
| | 4-4 | R-4 | 95 | 35 | 18 | 2.6 | 110 | 11.2 |
| | 4-5 | R-5 | 105 | 34 | 18 | 2.5 | 100 | 11.3 |
| | 4-6 | R-6 | 100 | 33 | 17 | 2.7 | 120 | 10.8 |
| | 4-7 | R-7 | 95 | 34 | 19 | 2.6 | 110 | 11.3 |
| | 4-8 | R-8 | 95 | 35 | 17 | 2.7 | 100 | 11.2 |
| | 4-9 | R-9 | 100 | 34 | 17 | 2.6 | 110 | 11.1 |
| | 4-10 | R-10 | 100 | 33 | 18 | 2.5 | 120 | 11.4 |
| | 4-11 | R-11 | 100 | 34 | 16 | 2.6 | 120 | 10.9 |
| | 4-12 | R-12 | 95 | 35 | 18 | 2.5 | 110 | 11.1 |
| | 4-13 | R-13 | 105 | 34 | 17 | 2.7 | 120 | 11.3 |
| | 4-14 | R-14 | 100 | 35 | 18 | 2.6 | 100 | 11.2 |
| | 4-15 | R-15 | 95 | 35 | 17 | 2.4 | 110 | 11.0 |
| | 4-16 | R-16 | 95 | 35 | 19 | 2.5 | 120 | 10.8 |
| | 4-17 | R-17 | 100 | 34 | 17 | 2.7 | 100 | 10.9 |
| | 4-18 | R-18 | 95 | 33 | 16 | 2.6 | 110 | 11.2 |
| | 4-19 | R-19 | 95 | 35 | 18 | 2.5 | 120 | 11.3 |
| | 4-20 | R-20 | 100 | 34 | 17 | 2.6 | 110 | 11.1 |
| | 4-21 | R-21 | 100 | 35 | 17 | 2.7 | 110 | 11.0 |
| | 4-22 | R-22 | 95 | 34 | 16 | 2.5 | 110 | 11.3 |
| | 4-23 | R-23 | 100 | 35 | 18 | 2.6 | 100 | 11.2 |
| | 4-24 | R-24 | 95 | 35 | 17 | 2.5 | 110 | 11.4 |
| | 4-25 | R-25 | 100 | 34 | 19 | 2.6 | 120 | 11.5 |
| | 4-26 | R-26 | 100 | 34 | 17 | 2.6 | 110 | 10.7 |
| | 4-27 | R-27 | 95 | 33 | 19 | 2.4 | 110 | 11.1 |
| | 4-28 | R-28 | 100 | 34 | 18 | 2.6 | 110 | 11.3 |
| | 4-29 | R-29 | 100 | 34 | 16 | 2.5 | 110 | 11.3 |
| | 4-30 | R-30 | 95 | 35 | 17 | 2.6 | 110 | 11.1 |

TABLE 8

| | | | | Optimal | | | | |
|---|---|---|---|---|---|---|---|---|
| | | Resist | PEB temp. | exposure dose | EL | LWR | DOF | Collapse limit |
| | | composition | (° C.) | (mJ/cm²) | (%) | (nm) | (nm) | (nm) |
| Example | 4-31 | R-31 | 100 | 34 | 19 | 2.6 | 110 | 11.2 |
| | 4-32 | R-32 | 100 | 34 | 17 | 2.5 | 100 | 11.3 |
| | 4-33 | R-33 | 100 | 35 | 18 | 2.4 | 110 | 10.8 |
| | 4-34 | R-34 | 95 | 36 | 17 | 2.6 | 120 | 11.1 |
| | 4-35 | R-35 | 100 | 34 | 16 | 2.4 | 110 | 11.0 |
| | 4-36 | R-36 | 95 | 36 | 19 | 2.6 | 110 | 11.3 |
| | 4-37 | R-37 | 100 | 35 | 18 | 2.5 | 120 | 11.2 |
| | 4-38 | R-38 | 100 | 36 | 18 | 2.4 | 110 | 11.3 |
| | 4-39 | R-39 | 95 | 35 | 17 | 2.6 | 100 | 11.1 |
| | 4-40 | R-40 | 100 | 36 | 16 | 2.5 | 110 | 11.2 |
| | 4-41 | R-41 | 95 | 34 | 18 | 2.5 | 110 | 11.4 |
| | 4-42 | R-42 | 95 | 33 | 17 | 2.7 | 120 | 11.1 |
| | 4-43 | R-43 | 100 | 34 | 19 | 2.5 | 110 | 10.7 |
| | 4-44 | R-44 | 100 | 36 | 17 | 2.6 | 100 | 11.1 |
| | 4-45 | R-45 | 95 | 34 | 18 | 2.4 | 120 | 11.3 |
| | 4-46 | R-46 | 95 | 35 | 17 | 2.5 | 110 | 11.4 |
| | 4-47 | R-47 | 100 | 36 | 17 | 2.7 | 110 | 10.9 |
| | 4-48 | R-48 | 95 | 35 | 18 | 2.6 | 120 | 11.0 |
| | 4-49 | R-49 | 95 | 34 | 18 | 2.7 | 110 | 10.6 |
| | 4-50 | R-50 | 100 | 34 | 18 | 2.6 | 110 | 11.0 |
| | 4-51 | R-51 | 95 | 33 | 17 | 2.5 | 120 | 11.1 |
| | 4-52 | R-52 | 95 | 34 | 16 | 2.4 | 110 | 11.2 |
| | 4-53 | R-53 | 100 | 35 | 18 | 2.5 | 110 | 11.3 |

TABLE 9

| | | | | Optimal | | | | Collapse |
|---|---|---|---|---|---|---|---|---|
| | | Resist | PEB temp. | exposure dose | EL | LWR | DOF | limit |
| | | composition | (° C.) | (mJ/cm²) | (%) | (nm) | (nm) | (nm) |
| Comparative | 4-1 | CR-1 | 100 | 38 | 13 | 3 | 90 | 12.1 |
| Example | 4-2 | CR-2 | 95 | 39 | 12 | 3.1 | 90 | 12.7 |
| | 4-3 | CR-3 | 100 | 37 | 14 | 3.2 | 80 | 12.2 |
| | 4-4 | CR-4 | 105 | 38 | 14 | 3.1 | 90 | 12.4 |
| | 4-5 | CR-5 | 100 | 39 | 14 | 3 | 80 | 12.6 |
| | 4-6 | CR-6 | 95 | 41 | 12 | 2.9 | 90 | 12.4 |
| | 4-7 | CR-7 | 100 | 38 | 12 | 3.2 | 90 | 13.2 |
| | 4-8 | CR-8 | 105 | 39 | 14 | 3.1 | 90 | 12.6 |
| | 4-9 | CR-9 | 95 | 40 | 14 | 3.1 | 80 | 13.2 |
| | 4-10 | CR-10 | 100 | 38 | 12 | 3 | 90 | 13.3 |
| | 4-11 | CR-11 | 95 | 39 | 13 | 3.1 | 90 | 12.7 |
| | 4-12 | CR-12 | 100 | 37 | 14 | 3.2 | 90 | 12.6 |
| | 4-13 | CR-13 | 100 | 41 | 12 | 3.2 | 90 | 13.1 |
| | 4-14 | CR-14 | 95 | 38 | 13 | 3.1 | 90 | 12.6 |
| | 4-15 | CR-15 | 105 | 40 | 12 | 3.1 | 90 | 12.8 |
| | 4-16 | CR-16 | 100 | 39 | 12 | 3 | 90 | 12.4 |
| | 4-17 | CR-17 | 100 | 38 | 11 | 3.1 | 80 | 13.1 |
| | 4-18 | CR-18 | 95 | 39 | 13 | 2.9 | 80 | 13.1 |
| | 4-19 | CR-19 | 100 | 37 | 12 | 3.1 | 90 | 12.5 |
| | 4-20 | CR-20 | 95 | 39 | 14 | 3 | 80 | 13.1 |
| | 4-21 | CR-21 | 100 | 42 | 13 | 2.9 | 90 | 12.4 |
| | 4-22 | CR-22 | 100 | 39 | 15 | 3 | 90 | 13.1 |
| | 4-23 | CR-23 | 100 | 38 | 13 | 3.2 | 80 | 12.7 |
| | 4-24 | CR-24 | 95 | 41 | 15 | 3.3 | 90 | 12.6 |
| | 4-25 | CR-25 | 100 | 39 | 12 | 3.1 | 90 | 12.6 |
| | 4-26 | CR-26 | 105 | 38 | 14 | 3.2 | 80 | 13.1 |
| | 4-27 | CR-27 | 100 | 39 | 13 | 3.1 | 90 | 12.4 |
| | 4-28 | CR-28 | 95 | 37 | 15 | 3.3 | 90 | 12.6 |
| | 4-29 | CR-29 | 100 | 41 | 14 | 3.2 | 90 | 12.3 |
| | 4-30 | CR-30 | 95 | 39 | 13 | 3.1 | 90 | 12.6 |
| | 4-31 | CR-31 | 100 | 38 | 12 | 3 | 90 | 12.4 |
| | 4-32 | CR-32 | 95 | 40 | 13 | 3.3 | 80 | 13.1 |
| | 4-33 | CR-33 | 95 | 39 | 14 | 3.5 | 90 | 12.9 |
| | 4-34 | CR-34 | 100 | 38 | 13 | 3.2 | 80 | 13.1 |
| | 4-35 | CR-35 | 100 | 37 | 15 | 3.4 | 90 | 12.9 |
| | 4-36 | CR-36 | 105 | 39 | 13 | 3.5 | 70 | 13.1 |
| | 4-37 | CR-37 | 95 | 38 | 14 | 3.6 | 70 | 13.3 |
| | 4-38 | CR-38 | 100 | 40 | 13 | 3.3 | 70 | 13.2 |
| | 4-39 | CR-39 | 95 | 38 | 13 | 3.5 | 70 | 13.3 |
| | 4-40 | CR-40 | 100 | 37 | 14 | 3.6 | 70 | 14.1 |

From the results shown in Tables 7-9, it is revealed that the inventive chemically amplified resist composition comprising a base polymer containing repeat units derived from an onium salt monomer has a high sensitivity, and excellent EL, LWR and DOF. The resist composition is also confirmed to have a low collapse resistance value, and resistance to pattern collapse in fine pattern formation. This demonstrates that chemically amplified resist compositions are suitable as materials for EUV lithography.

### [5] EUV Lithography Test (2)

### Examples 5-1 to 5-53 and Comparative Examples 5-1 to 5-40

Each of the chemically amplified resist compositions (R-1 to R-53, CR-1 to CR-10) shown in Tables 4 to 6 was spin coated on a silicon substrate having a 20-nm coating of silicon-containing spin-on hard mask SHB-A940 (Shin-Etsu Chemical Co., Ltd., silicon content 43 wt %) and prebaked on a hotplate at 105° C. for 60 seconds to form a resist film of 50 nm thick. Using an EUV scanner NXE3400 (ASML, NA 0.33, a 0.9/0.6, quadrupole illumination), the resist film was exposed to EUV through a mask bearing a hole pattern having a pitch of 46 nm (on-wafer size) and +20% bias. The resist film was baked (PEB) on a hotplate at the temperature shown in Tables 7 and 8 for 60 seconds and developed in a 2.38 wt % TMAH aqueous solution for 30 seconds to form a hole pattern with a size of 23 nm.

The hole pattern was observed under CD-SEM (CG6300, Hitachi High-Technologies Corp.). The exposure dose that provides a hole pattern having a size of 23 nm is reported as sensitivity. The size of 50 holes was measured, from which a 3-fold value (3σ) of standard deviation (σ) was computed and reported as size variation or CDU. The results are shown in Tables 10 to 12.

TABLE 10

| | | | | Optimal | |
|---|---|---|---|---|---|
| | | Resist | PEB temp. | exposure dose | CDU |
| | | composition | (° C.) | (mJ/cm²) | (nm) |
| Example | 5-1 | R-1 | 95 | 23 | 2.2 |
| | 5-2 | R-2 | 95 | 23 | 2.4 |
| | 5-3 | R-3 | 90 | 22 | 2.3 |
| | 5-4 | R-4 | 90 | 23 | 2.4 |
| | 5-5 | R-5 | 90 | 24 | 2.3 |
| | 5-6 | R-6 | 95 | 23 | 2.5 |
| | 5-7 | R-7 | 95 | 24 | 2.2 |
| | 5-8 | R-8 | 90 | 23 | 2.4 |
| | 5-9 | R-9 | 95 | 25 | 2.5 |
| | 5-10 | R-10 | 95 | 23 | 2.6 |
| | 5-11 | R-11 | 95 | 23 | 2.3 |
| | 5-12 | R-12 | 90 | 23 | 2.4 |
| | 5-13 | R-13 | 90 | 24 | 2.3 |
| | 5-14 | R-14 | 90 | 25 | 2.4 |
| | 5-15 | R-15 | 90 | 23 | 2.3 |
| | 5-16 | R-16 | 85 | 23 | 2.4 |
| | 5-17 | R-17 | 95 | 22 | 2.5 |
| | 5-18 | R-18 | 95 | 24 | 2.3 |
| | 5-19 | R-19 | 90 | 24 | 2.4 |
| | 5-20 | R-20 | 95 | 23 | 2.5 |
| | 5-21 | R-21 | 95 | 23 | 2.3 |
| | 5-22 | R-22 | 95 | 24 | 2.5 |
| | 5-23 | R-23 | 95 | 25 | 2.3 |
| | 5-24 | R-24 | 95 | 23 | 2.5 |
| | 5-25 | R-25 | 95 | 25 | 2.4 |
| | 5-26 | R-26 | 95 | 24 | 2.4 |
| | 5-27 | R-27 | 95 | 23 | 2.3 |
| | 5-28 | R-28 | 95 | 25 | 2.2 |
| | 5-29 | R-29 | 90 | 23 | 2.4 |
| | 5-30 | R-30 | 95 | 23 | 2.3 |

TABLE 11

| | | | | Optimal | |
|---|---|---|---|---|---|
| | | Resist | PEB temp. | exposure dose | CDU |
| | | composition | (° C.) | (mJ/cm²) | (nm) |
| Example | 5-31 | R-31 | 95 | 24 | 2.3 |
| | 5-32 | R-32 | 95 | 23 | 2.3 |
| | 5-33 | R-33 | 95 | 25 | 2.2 |
| | 5-34 | R-34 | 90 | 24 | 2.3 |
| | 5-35 | R-35 | 95 | 25 | 2.4 |
| | 5-36 | R-36 | 95 | 23 | 2.5 |
| | 5-37 | R-37 | 90 | 23 | 2.4 |
| | 5-38 | R-38 | 90 | 24 | 2.3 |
| | 5-39 | R-39 | 95 | 23 | 2.5 |
| | 5-40 | R-40 | 95 | 22 | 2.2 |
| | 5-41 | R-41 | 95 | 23 | 2.4 |
| | 5-42 | R-42 | 90 | 24 | 2.3 |
| | 5-43 | R-43 | 95 | 23 | 2.4 |
| | 5-44 | R-44 | 95 | 24 | 2.5 |
| | 5-45 | R-45 | 90 | 25 | 2.5 |
| | 5-46 | R-46 | 95 | 24 | 2.3 |
| | 5-47 | R-47 | 90 | 23 | 2.3 |
| | 5-48 | R-48 | 90 | 24 | 2.5 |
| | 5-49 | R-49 | 95 | 25 | 2.4 |
| | 5-50 | R-50 | 90 | 23 | 2.5 |
| | 5-51 | R-51 | 95 | 24 | 2.3 |
| | 5-52 | R-52 | 95 | 24 | 2.4 |
| | 5-53 | R-53 | 95 | 24 | 2.5 |

TABLE 12

| | | | | Optimal | |
|---|---|---|---|---|---|
| | | Resist | PEB temp. | exposure dose | CDU |
| | | composition | (° C.) | (mJ/cm²) | (nm) |
| Comparative | 5-1 | CR-1 | 95 | 29 | 2.9 |
| Example | 5-2 | CR-2 | 90 | 28 | 2.8 |
| | 5-3 | CR-3 | 90 | 28 | 2.9 |
| | 5-4 | CR-4 | 95 | 28 | 2.8 |
| | 5-5 | CR-5 | 90 | 29 | 2.9 |
| | 5-6 | CR-6 | 95 | 28 | 2.7 |
| | 5-7 | CR-7 | 95 | 27 | 2.8 |
| | 5-8 | CR-8 | 95 | 28 | 2.8 |
| | 5-9 | CR-9 | 90 | 28 | 2.9 |
| | 5-10 | CR-10 | 95 | 27 | 2.7 |
| | 5-11 | CR-11 | 95 | 29 | 2.9 |
| | 5-12 | CR-12 | 90 | 29 | 2.8 |
| | 5-13 | CR-13 | 90 | 28 | 2.8 |
| | 5-14 | CR-14 | 90 | 29 | 2.9 |
| | 5-15 | CR-15 | 95 | 29 | 3 |
| | 5-16 | CR-16 | 85 | 30 | 2.8 |
| | 5-17 | CR-17 | 95 | 28 | 2.8 |
| | 5-18 | CR-18 | 95 | 27 | 2.9 |
| | 5-19 | CR-19 | 90 | 29 | 2.8 |
| | 5-20 | CR-20 | 95 | 27 | 2.9 |
| | 5-21 | CR-21 | 95 | 28 | 2.7 |
| | 5-22 | CR-22 | 95 | 29 | 2.8 |
| | 5-23 | CR-23 | 95 | 28 | 2.9 |
| | 5-24 | CR-24 | 95 | 28 | 2.9 |
| | 5-25 | CR-25 | 95 | 28 | 2.8 |
| | 5-26 | CR-26 | 95 | 29 | 2.9 |
| | 5-27 | CR-27 | 95 | 27 | 2.8 |
| | 5-28 | CR-28 | 95 | 29 | 2.7 |
| | 5-29 | CR-29 | 90 | 29 | 2.8 |
| | 5-30 | CR-30 | 90 | 28 | 2.9 |
| | 5-31 | CR-31 | 95 | 27 | 2.9 |
| | 5-32 | CR-32 | 90 | 30 | 2.8 |
| | 5-33 | CR-33 | 95 | 28 | 2.9 |
| | 5-34 | CR-34 | 95 | 29 | 2.9 |
| | 5-35 | CR-35 | 95 | 29 | 2.9 |
| | 5-36 | CR-36 | 95 | 28 | 2.9 |
| | 5-37 | CR-37 | 90 | 30 | 3.1 |
| | 5-38 | CR-38 | 90 | 30 | 3.2 |
| | 5-39 | CR-39 | 90 | 31 | 3.1 |
| | 5-40 | CR-40 | 90 | 31 | 3.1 |

It is demonstrated in Tables 10 to 12 that chemically amplified resist compositions within the scope of the invention exhibit a high sensitivity and improved CDU.

### [6] Evaluation of Dry Etch Resistance

### Examples 6-1 to 6-50 and Comparative Examples 6-1 to 6-38

2 g of each of the polymers shown in Tables 1 and 2 (Polymers P-1 to P-50 and Comparative Polymers CP-1 to CP-10) was dissolved in 10 g of cyclohexanone. The polymer solution was filtered through a filter with a pore size of 0.2 μm, and spin-coated on a Si substrate to form a 300 nm-thick film thereon, and the film was tested under the following conditions.

Etching Test with CHF₃/CF₄-Based Gas:

A polymer film thickness difference before and after etching was determined using a dry etching apparatus TE-8500 P manufactured by Tokyo Electron Limited.

The etching conditions are shown below.
Chamber pressure: 40 Pa
RF power: 1,000 W
Gap: 9 mm
CHF₃ gas flow rate: 30 mL/min
CF₄ gas flow rate: 30 mL/min
Ar gas flow rate: 100 mL/min
Time: 60 sec

In this evaluation, a smaller film thickness difference, i.e. a smaller amount of decrease, indicates higher etch resistance.

The results of dry etch resistance evaluation are shown in Tables 13 to 15.

TABLE 13

| | | Polymer | CHF₃/CF₄ gas etching rate (nm/min) |
|---|---|---|---|
| Example | 6-1 | P-1 | 94 |
| | 6-2 | P-2 | 95 |
| | 6-3 | P-3 | 97 |
| | 6-4 | P-4 | 94 |
| | 6-5 | P-5 | 95 |
| | 6-6 | P-6 | 94 |
| | 6-7 | P-7 | 96 |
| | 6-8 | P-8 | 95 |
| | 6-9 | P-9 | 95 |
| | 6-10 | P-10 | 94 |
| | 6-11 | P-11 | 96 |
| | 6-12 | P-12 | 97 |
| | 6-13 | P-13 | 94 |
| | 6-14 | P-14 | 96 |
| | 6-15 | P-15 | 95 |
| | 6-16 | P-16 | 96 |
| | 6-17 | P-17 | 95 |
| | 6-18 | P-18 | 94 |
| | 6-19 | P-19 | 97 |
| | 6-20 | P-20 | 95 |
| | 6-21 | P-21 | 94 |
| | 6-22 | P-22 | 97 |
| | 6-23 | P-23 | 95 |
| | 6-24 | P-24 | 97 |
| | 6-25 | P-25 | 98 |
| | 6-26 | P-26 | 95 |
| | 6-27 | P-27 | 95 |
| | 6-28 | P-28 | 97 |
| | 6-29 | P-29 | 95 |
| | 6-30 | P-30 | 96 |

TABLE 14

| | | Polymer | CHF₃/CF₄ gas etching rate (nm/min) |
|---|---|---|---|
| Example | 6-31 | P-31 | 95 |
| | 6-32 | P-32 | 94 |
| | 6-33 | P-33 | 95 |
| | 6-34 | P-34 | 95 |
| | 6-35 | P-35 | 97 |
| | 6-36 | P-36 | 94 |
| | 6-37 | P-37 | 96 |
| | 6-38 | P-38 | 96 |
| | 6-39 | P-39 | 96 |
| | 6-40 | P-40 | 96 |
| | 6-41 | P-41 | 98 |
| | 6-42 | P-42 | 95 |
| | 6-43 | P-43 | 94 |
| | 6-44 | P-44 | 97 |
| | 6-45 | P-45 | 95 |
| | 6-46 | P-46 | 94 |
| | 6-47 | P-47 | 96 |
| | 6-48 | P-48 | 96 |
| | 6-49 | P-49 | 95 |
| | 6-50 | P-50 | 96 |

TABLE 15

| | | Polymer | CHF₃/CF₄ gas etching rate (nm/min) |
|---|---|---|---|
| Comparative | 6-1 | CP-1 | 101 |
| Example | 6-2 | CP-2 | 102 |
| | 6-3 | CP-3 | 102 |
| | 6-4 | CP-4 | 104 |
| | 6-5 | CP-5 | 101 |
| | 6-6 | CP-6 | 102 |
| | 6-7 | CP-7 | 105 |
| | 6-8 | CP-8 | 103 |
| | 6-9 | CP-9 | 104 |
| | 6-10 | CP-10 | 101 |
| | 6-11 | CP-11 | 105 |
| | 6-12 | CP-12 | 103 |
| | 6-13 | CP-13 | 104 |
| | 6-14 | CP-14 | 109 |
| | 6-15 | CP-15 | 105 |
| | 6-16 | CP-16 | 106 |
| | 6-17 | CP-17 | 105 |
| | 6-18 | CP-18 | 106 |
| | 6-19 | CP-19 | 103 |
| | 6-20 | CP-20 | 102 |
| | 6-21 | CP-21 | 105 |
| | 6-22 | CP-22 | 102 |
| | 6-23 | CP-23 | 107 |
| | 6-24 | CP-24 | 105 |
| | 6-25 | CP-25 | 101 |
| | 6-26 | CP-26 | 103 |
| | 6-27 | CP-27 | 106 |
| | 6-28 | CP-28 | 108 |
| | 6-29 | CP-29 | 103 |
| | 6-30 | CP-30 | 105 |
| | 6-31 | CP-31 | 107 |
| | 6-32 | CP-32 | 109 |
| | 6-33 | CP-33 | 104 |
| | 6-34 | CP-34 | 106 |
| | 6-35 | CP-35 | 105 |
| | 6-36 | CP-36 | 106 |
| | 6-37 | CP-37 | 107 |
| | 6-38 | CP-38 | 114 |

It is evident from Tables 13 to 15 that the inventive polymers have good dry etch resistance, i.e., resistance to CHF₃/CF₄ gas etching.

Japanese Patent Application No. 2024-103103 is incorporated herein by reference.

Although some preferred embodiments have been described, many modifications and variations may be made thereto in light of the above teachings. It is therefore to be understood that the invention may be practiced otherwise than as specifically described without departing from the scope of the appended claims.

1. A sulfonium salt monomer having the formula (a): 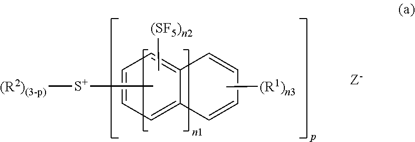
wherein p is 1, 2 or 3, n1 is 0 or 1, n2 is 1 or 2, n3 is 0, 1, 2 or 3, n2+n3 is from 1 to 5 when n1 is 0, n2+n3 is from 1 to 7 when n1 is 1,
R¹ is halogen, nitro group, cyano group, hydroxy group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, a plurality of R¹ may be identical or different and two R¹ may bond together to form a ring with the carbon atoms to which they are attached, when n3 is 2 or 3,
R² is halogen, or a C₁-C₃₀ hydrocarbyl group which may contain a heteroatom, two R² may be identical or different when p is 1, two of three substituents bonded to S⁺ may bond together to form a ring with a sulfur atom to which they are attached, and
Z⁻ is a carboxylate anion having an aromatic vinyl structure and iodine.

2. The sulfonium salt monomer of claim 1 which has the formula (a1): 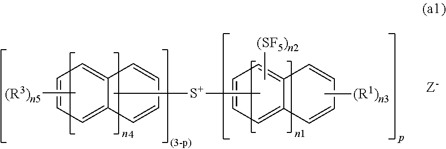
wherein p, n1 to n3, and R¹ and Z⁻ are as defined above,
n4 is 0 or 1, n5 is 0, 1, 2, 3, 4 or 5,
R³ is halogen, nitro group, cyano group, hydroxy group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, and a plurality of R³ may be identical or different and two R³ may bond together to form a ring with the carbon atoms to which they are attached, when n5 is an integer of 2 to 5.

3. The sulfonium salt monomer of claim 1, wherein Z⁻ is an anion having the formula (Z): 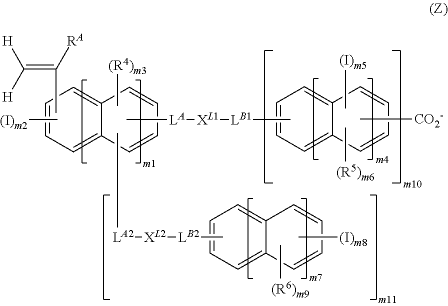
wherein m1 is 0 or 1, m2 is 0, 1, 2, 3 or 4, m3 is 0, 1, 2 or 3, m4 is 0 or 1, m5 is 0, 1, 2, 3 or 4, m6 is 0, 1, 2 or 3, m7 is 0 or 1, m8 is 1, 2, 3 or 4, m9 is 0, 1, 2 or 3, m10 is 0 or 1, m1 is 0 or 1, m2+m3+m11 is from 0 to 4 when m1 is 0, m2+m3+m11 is from 0 to 6 when m1 is 1, m5+m6 is from 0 to 4 when m4 is 0, m5+m6 is from 0 to 6 when m4 is 1, m8+m9 is from 0 to 5 when m7 is 0, m8+m9 is from 0 to 7 when m7 is 1, m2+m5+m8 is from 1 to 4,
R^{A} is hydrogen, fluorine, methyl group or trifluoromethyl group,
R⁴, R⁵ and R⁶ are each independently halogen exclusive of iodine, nitro group, hydroxy group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, a plurality of R⁴ may be identical or different and two R⁴ may bond together to form a ring with the carbon atoms to which they are attached, when m3 is 2 or 3, a plurality of R⁵ may be identical or different and two R⁵ may bond together to form a ring with the carbon atoms to which they are attached, when m6 is 2 or 3, a plurality of R⁶ may be identical or different and two R⁶ may bond together to form a ring with the carbon atoms to which they are attached, when m9 is 2 or 3,
L^{A1}, L^{A2}, L^{B1} and L^{B2} are each independently a single bond, ether bond, carbonyl group, ester bond, sulfonic ester bond, amide bond, sulfonic amide bond, carbonate bond or carbamate bond,
X^{L1} and X^{L2} are each independently a single bond, or a C₁-C₄₀ hydrocarbylene group which may contain a heteroatom.

4. A sulfonium salt quencher comprising the sulfonium salt monomer of claim 1.

5. A polymer comprising repeat units derived from the sulfonium salt quencher of claim 4.

6. The polymer of claim 5, further comprising repeat units having the formula (b1) or (b2): 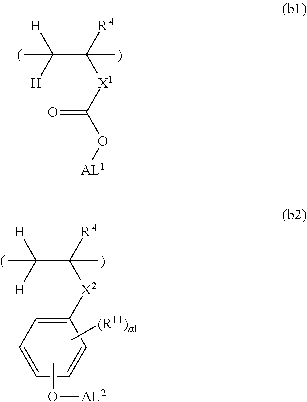
wherein R^{A} is each independently hydrogen, fluorine, methyl group or trifluoromethyl group,
X¹ is a single bond, phenylene group, naphthylene group, *—C(═O)—O—X¹¹— or *—C(═O)—NH—X¹¹—, the phenylene group or naphthylene group may be substituted with hydroxy group, nitro group, cyano group, a C₁-C₁₀ saturated hydrocarbyl group which may contain fluorine, a C₁-C₁₀ saturated hydrocarbyloxy group which may contain fluorine, or halogen, X¹¹ is a C₁-C₁₀ saturated hydrocarbylene group, phenylene group, or naphthylene group, the saturated hydrocarbylene group may contain hydroxy group, ether bond, ester bond or lactone ring,
X² is a single bond, *—C(═O)—O— or *—C(═O)—NH—,
* designates a point of attachment to the carbon atom in the backbone,
R¹¹ is halogen, cyano group, hydroxy group, nitro group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, a plurality of R¹¹ may be identical or different when a1 is 2 or more,
A^{L} and AL² are each independently an acid labile group, and
a1 is 0, 1, 2, 3 or 4.

7. The polymer of claim 5, further comprising repeat units having the formula (b3): 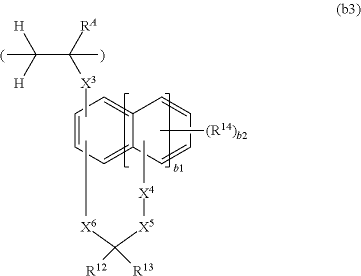
wherein b1 is 0 or 1, b2 is 0, 1, 2 or 3 when b1 is 0, b2 is 0, 1, 2, 3, 4 or 5 when b1 is 1,
R^{A} is hydrogen, fluorine, methyl group or trifluoromethyl group,
X³ is a single bond, *—C(═O)—O— or *—C(═O)—NH—, * designates a point of attachment to the carbon atom in the backbone,
R¹² and R¹³ are each independently hydrogen or a C₁-C₂₀ hydrocarbyl group which may contain a heteroatom, R¹² and R¹³ may bond together to form a ring with the carbon atoms to which they are attached,
R¹⁴ is halogen, hydroxy group, cyano group, nitro group, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{14A})(R^{14B}), R^{14A} and R^{14B} are each independently hydrogen or a C₁-C₆ hydrocarbyl group, a plurality of R¹⁴ may be identical or different and a plurality of R¹⁴ may bond together to form a ring with the carbon atoms to which they are attached, when b2 is 2 or more,
X⁴ is a single bond, C₁-C₄ aliphatic hydrocarbylene group, carbonyl group, sulfonyl group or a group obtained by combining the foregoing, and
X⁵ and X⁶ are each independently oxygen or sulfur,
provided that X⁴ and X⁶ are attached to adjacent carbon atoms on the aromatic ring.

8. The polymer of claim 5, further comprising repeat units having the formula (c): 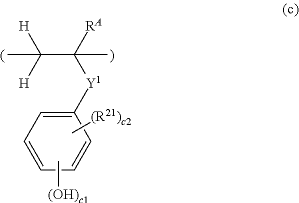
wherein R^{A} is hydrogen, fluorine, methyl group, or trifluoromethyl group,
Y¹ is a single bond, *—C(═O)—O— or *—C(═O)—NH—, * designates a point of attachment to the carbon atom in the backbone,
R²¹ is halogen, nitro group, cyano group, carboxy group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom,
c1 is 1, 2, 3 or 4, and c2 is 0, 1, 2 or 3,
provided that c1+c2 is from 1 to 5.

9. The polymer of claim 5, further comprising repeat units derived from an onium salt monomer containing a fluorosulfonate anion having a polymerizable group and at least one iodine, and a sulfonium cation.

10. The polymer according to claim 5, further comprising repeat units having formula (e): 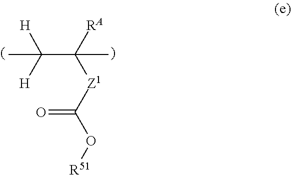
wherein R^{A} is hydrogen, fluorine, methyl group or trifluoromethyl group,
Z¹ is a single bond, phenylene group, naphthylene group, *—C(═O)—O—Z¹¹— or *—C(═O)—NH—Z¹¹—, the phenylene group or naphthylene group may be substituted with hydroxy group, nitro group, cyano group, a C₁-C₁₀ saturated hydrocarbyl group which may contain fluorine, a C₁-C₁₀ saturated hydrocarbyloxy group which may contain fluorine, or halogen, * designates a point of attachment to a carbon atom in the backbone, Z¹¹ is a C₁-C₁₀ saturated hydrocarbylene group, phenylene group or naphthylene group, the saturated hydrocarbylene group may contain hydroxy group, ether bond, ester bond or lactone ring, and
R⁵¹ is hydrogen, or a C₁-C₂₀ group containing at least one structure selected from a hydroxy group exclusive of phenolic hydroxy group, a cyano group, carbonyl group, carboxy group, ether bond, ester bond, sulfonic ester bond, carbonate bond, lactone ring, sultone ring, and carboxylic anhydride (—C(═O)—O—C(═O)—).

11. A chemically amplified resist composition comprising (A) a base polymer containing the polymer of claim 5.

12. The chemically amplified resist composition according to claim 11, further comprising (B) an organic solvent.

13. The chemically amplified resist composition of claim 11, further comprising (C) a quencher.

14. The chemically amplified resist composition of claim 11, further comprising (D) an acid generator.

15. The chemically amplified resist composition of claim 11, further comprising (E) a surfactant.

16. A pattern forming process comprising the steps of applying the chemically amplified resist composition of claim 11 onto a substrate to form a resist film thereon, exposing the resist film to high-energy radiation, and developing the exposed resist film in a developer.

17. The pattern forming process of claim 16, wherein the high-energy radiation is ArF excimer laser having a wavelength 193 nm, KrF excimer laser having a wavelength 248 nm, an electron beam, or extreme ultraviolet having a wavelength 3 to 15 nm.
